(19) United States
(12) Patent Application Publication
(10) Pub. No.: US 2026/0055430 A1
(43) Pub. Date: Feb. 26, 2026

(54) LENTIVIRUS WITH ALTERED INTEGRASE ACTIVITY

(71) Applicant: Flagship Pioneering Innovations VI, LLC, Cambridge, MA (US)

(72) Inventors: Jacob Rosenblum Rubens, Cambridge, MA (US); Robert James Citorik, Somerville, MA (US); Yanfang Fu, Arlington, MA (US); Cecilia Giovanna Silvia Cotta-Ramusino, Cambridge, MA (US); William Edward Salomon, West Roxbury, MA (US); Zi Jun Wang, Arlington, MA (US)

(21) Appl. No.: 18/280,749

(22) PCT Filed: Mar. 8, 2022
(86) PCT No.: PCT/US2022/071018

§ 371 Date: Sep. 7, 2023

Related U.S. Application Data
(60) Provisional application No. 63/158,187, filed on Mar. 8, 2021.
(60) Provisional application No. 63/193,546, filed on May 26, 2021.

(51) Int. Cl. C12N 15/90; C12N 9/12; C12N 9/22

(52) U.S. Cl. CPC C12N 15/90; C12N 9/1276; C12N 9/22; C12N 2740/16222; C12Y 207/07049; C12Y 301/22

(57) ABSTRACT

Among other things, provided herein are systems that replace the natural random integration activity of a retrovirus with site-specific integration machinery. This approach allows for a more precise targeting of a gene of interest into a human genome, e.g., for therapeutic purposes. The system may include integration-deficient retrovirus (e.g., lentivirus) (IDLV), in which the natural integration activity has been reduced (e.g., by mutation to the viral integrase polypeptide). Instead, the system may comprise a site-specific recombinase (e.g., a serine recombinase, e.g., a serine integrase) capable of directing insertion of a template DNA, or portion thereof, into a desired site in the human genome.

### CROSS-REFERENCE TO RELATED APPLICATIONS

This application is a U.S. National Phase Application under 35 U.S.C. § 371 of International Application No. PCT Utility Application No. PCT/US2022/071018, filed Mar. 8, 2022, which claims the benefit of U.S. Provisional Application Nos. 63/158,187, filed Mar. 8, 2021; and 63/193,546, filed May 26, 2021. The contents of the aforementioned applications are hereby incorporated by reference in their entirety.

### SEQUENCE LISTING

The instant application contains a Sequence Listing which has been submitted electronically in ASCII format and is hereby incorporated by reference in its entirety. Said ASCII copy, created on May 25, 2021, is named V2065-7018WO_SL.txt and is 72,577,024 bytes in size.

### SUMMARY OF THE INVENTION

This disclosure relates to novel compositions, systems and methods for altering a genome at one or more locations in a host cell, tissue or subject, in vivo, in vitro, or ex vivo. In particular, the invention features compositions, systems and methods for the introduction of exogenous genetic elements into a target cell genome using a recombinase polypeptide (e.g., a serine recombinase, e.g., as described herein), wherein the exogenous genetic element is introduced into the target cell by an integration-deficient retroviral vector. In some embodiments, a recombinase as described herein is an integrase. In some embodiments, a serine recombinase as described herein is a serine integrase.

### ENUMERATED EMBODIMENTS

1. A system for modifying DNA comprising:
a) a template RNA comprising a DNA recognition sequence, or a DNA molecule encoding the template RNA;
b) a retroviral (e.g., lentiviral) structural polypeptide domain (e.g., gag), or a nucleic acid molecule encoding the retroviral (e.g., lentiviral) structural polypeptide domain;
c) a retroviral (e.g., lentiviral) reverse transcriptase polypeptide domain (e.g., pol or an polypeptide comprising an amino acid sequence as listed in Table 11 or 12, or an amino acid sequence having at least 70%, 75%, 80%, 85%, 90%, 95%, 96%, 97%, 98%, or 99% identity thereto) capable of reverse transcribing the template RNA, thereby producing a template DNA, or a nucleic acid molecule encoding the retroviral (e.g., lentiviral) reverse transcriptase polypeptide domain;
wherein b) and c) together are integration-deficient;
d) a serine recombinase (e.g., serine integrase) polypeptide domain comprising an amino acid sequence of any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto, wherein the serine recombinase polypeptide domain binds the DNA recognition sequence and is capable of integrating the template DNA into the target DNA; or a nucleic acid molecule encoding the serine recombinase polypeptide domain, and
e) a retroviral (e.g., lentiviral) envelope polypeptide domain (e.g., env), or a nucleic acid molecule encoding the retroviral (e.g., lentiviral) envelope polypeptide domain;
wherein b), c), d), and e) are optionally part of the same polypeptide.

2. A system for modifying DNA comprising:
a) a template RNA comprising a DNA recognition sequence that is recognized by a serine recombinase (e.g., serine integrase) polypeptide domain that comprises an amino acid sequence of any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto, or a DNA molecule encoding the template RNA;
b) a retroviral (e.g., lentiviral) structural polypeptide domain (e.g., gag);
c) a retroviral (e.g., lentiviral) reverse transcriptase polypeptide domain (e.g., pol, e.g., as listed in Table 11 or 12) capable of reverse transcribing the template RNA, thereby producing a template DNA; and
d) a retroviral (e.g., lentiviral) envelope polypeptide domain (e.g., env), or a nucleic acid molecule encoding the retroviral (e.g., lentiviral) envelope polypeptide domain;
wherein b) and c) are substantially unable to integrate the template DNA into a target DNA; and
wherein b), c), and d) are optionally part of the same polypeptide.

3. The system of embodiment 2, which further comprises:
e) the serine recombinase (e.g., serine integrase) polypeptide domain, wherein the serine recombinase polypeptide domain binds the DNA recognition sequence and is capable of integrating the template DNA into the target DNA, or a nucleic acid molecule encoding the serine recombinase polypeptide domain.

4. A system for modifying DNA comprising:
a) a template RNA comprising a DNA recognition sequence and a heterologous object sequence encoding a therapeutic effector (e.g., wherein the therapeutic effector comprising a polypeptide or functional nucleic acid molecule, e.g., an siRNA, lncRNA, asRNA, miRNA, or any other ncRNA), or a DNA molecule encoding the template RNA;
b) a retroviral (e.g., lentiviral) structural polypeptide domain (e.g., gag);
c) a retroviral (e.g., lentiviral) reverse transcriptase polypeptide domain (e.g., pol, e.g., as listed in Table 11 or 12) capable of reverse transcribing the template RNA, thereby producing a template DNA;
wherein b) and c) are substantially unable to integrate the template DNA into a target DNA;
d) a serine recombinase (e.g., serine integrase) polypeptide domain, wherein the serine recombinase polypeptide domain binds the DNA recognition sequence and is capable of integrating the template DNA into the target DNA; or a nucleic acid molecule encoding the serine recombinase polypeptide domain; and
e) a retroviral (e.g., lentiviral) envelope polypeptide domain (e.g., env), or a nucleic acid molecule encoding the retroviral (e.g., lentiviral) envelope polypeptide domain;
wherein b), c), d), and e) are optionally part of the same polypeptide.

5. The system of embodiment 2, wherein the serine recombinase (e.g., serine integrase) polypeptide domain has less than 80% (e.g., less than 80%, 75%, 70%, 60%, 50%, 40%, 30%, 20%, 10%, or 5%) amino acid sequence identity to phiC31 phage integrase (e.g., a phiC31 integrase having the amino acid sequence as listed in NCBI Accession No. NC_001978.3).

6. The system of embodiment 2, wherein the serine recombinase (e.g., serine integrase) polypeptide domain does not comprise a recombinase (e.g., integrase) from a Streptomyces phage, e.g., the Streptomyces temperate phage phiC31, e.g., having the amino acid sequence as listed in NCBI Accession No. NC_001978.3.

7. A system for modifying DNA comprising:
a) a template RNA comprising a DNA recognition sequence, or a DNA molecule encoding the template RNA;
b) a retroviral (e.g., lentiviral) structural polypeptide domain (e.g., gag);
c) a retroviral (e.g., lentiviral) reverse transcriptase polypeptide domain (e.g., pol, e.g., as listed in Table 11 or 12) capable of reverse transcribing the template RNA, thereby producing a template DNA;
wherein b) and c) are substantially unable to integrate the template DNA into a DNA; and
d) a serine recombinase (e.g., serine integrase) polypeptide domain, wherein the serine recombinase polypeptide domain binds the DNA recognition sequence and is capable of integrating the template DNA into a target DNA, and
e) a retroviral (e.g., lentiviral) envelope polypeptide domain (e.g., env), or a nucleic acid molecule encoding the retroviral (e.g., lentiviral) envelope polypeptide domain;
wherein b), c), d), and e) are optionally part of the same polypeptide;
wherein the DNA recognition sequence of the template DNA is capable of being recombined by the serine recombinase polypeptide domain with a cognate DNA recognition sequence in a naturally occurring human genome and/or in Genome Reference Consortium Human Build 38 (GRCh38); and
wherein the target DNA comprises the cognate DNA recognition sequence.

8. The system of embodiment 7, wherein the target DNA is comprised in a human genome.

9. The system of embodiment 8, wherein the target DNA is present at least once in the human genome, e.g., at least 1, 2, 3, 4, 5, 10, 20, 30, 40, 50, 100, 200, 300, 400, 500, 1000, 2000, 3000, 4000, 5000, 6000, 7000, 8000, 9000, or at least 10000 occurrences.

10. The system of embodiment 8, wherein the target DNA is present no more than 2 times (e.g., no more than 1, 2, 3, 4, 5, 6, 8, 9, 10, 50, 100, 200, 300, 400, 500, 600, 700, 800, 900, or 1000 times) in the human genome.

11. A system for modifying DNA comprising:
a) a template RNA comprising a DNA recognition sequence, or a DNA molecule encoding the template RNA;
b) a retroviral (e.g., lentiviral) structural polypeptide domain (e.g., gag);
c) a retroviral (e.g., lentiviral) reverse transcriptase polypeptide domain (e.g., pol, e.g., as listed in Table 11 or 12) capable of reverse transcribing the template RNA, thereby producing a template DNA;
wherein b) and c) are substantially unable to integrate the template DNA into a DNA; and
d) a serine recombinase (e.g., serine integrase) polypeptide domain, wherein the serine recombinase polypeptide domain binds the DNA recognition sequence and is capable of integrating the template DNA into a target DNA, and
e) a retroviral (e.g., lentiviral) envelope polypeptide domain (e.g., env), or a nucleic acid molecule encoding the retroviral (e.g., lentiviral) envelope polypeptide domain;
wherein b), c), d), and e) are optionally part of the same polypeptide;
wherein the serine recombinase polypeptide domain is capable of recombining the DNA recognition sequence of the template DNA with a cognate DNA recognition sequence in a naturally occurring human genome; and
wherein the target DNA comprises the cognate DNA recognition sequence.

12. The system of embodiment 11, wherein the target DNA is comprised in a human genome.

13. A system for modifying DNA comprising:
a) a template RNA comprising a DNA recognition sequence, or a DNA molecule encoding the template RNA;
b) a retroviral (e.g., lentiviral) structural polypeptide domain (e.g., gag);
c) a retroviral (e.g., lentiviral) reverse transcriptase polypeptide domain (e.g., pol, e.g., as listed in Table 11 or 12) capable of reverse transcribing the template RNA, thereby producing a template DNA, wherein the retroviral (e.g., lentiviral) reverse transcriptase polypeptide domain does not comprise a D64V mutation, or wherein the retroviral (e.g., lentiviral) reverse transcriptase polypeptide domain comprises a D116 or E152 mutation;
wherein b) and c) are substantially unable to integrate the template DNA into a DNA; and
d) a serine recombinase (e.g., serine integrase) polypeptide domain, wherein the serine recombinase polypeptide domain binds the DNA recognition sequence and is capable of integrating the template DNA into a target DNA,
e) a retroviral (e.g., lentiviral) envelope polypeptide domain (e.g., env), or a nucleic acid molecule encoding the retroviral (e.g., lentiviral) envelope polypeptide domain;
wherein b), c), d), and e) are optionally part of the same polypeptide;
wherein the DNA recognition sequence of the template DNA is capable of being recombined by the serine recombinase polypeptide domain with a cognate DNA recognition sequence in a naturally occurring human genome; and
wherein the target DNA comprises the cognate DNA recognition sequence.

14. A system for modifying DNA comprising:
a) template RNA comprising a DNA recognition sequence, or a DNA molecule encoding the template RNA,
b) a lentiviral structural polypeptide domain (e.g., gag);
c) a lentiviral reverse transcriptase polypeptide domain (e.g., pol, e.g., as listed in Table 11 or 12) capable of reverse transcribing the template RNA, thereby producing a template DNA;
wherein b) and c) are substantially unable to integrate the template DNA into a target DNA;
d) serine integrase polypeptide domain, or a nucleic acid molecule encoding the serine integrase polypeptide domain; and
e) a retroviral (e.g., lentiviral) envelope polypeptide domain (e.g., env), or a nucleic acid molecule encoding the retroviral (e.g., lentiviral) envelope polypeptide domain.

15. A system for modifying DNA comprising:
a) a template RNA comprising a first long terminal repeat (LTR), a second LTR, a heterologous object sequence encoding a therapeutic effector, positioned between the first LTR and the second LTR, a DNA recognition sequence, and optionally a primer binding site (PBS); or a DNA molecule encoding the template RNA;
b) a structural polypeptide domain (e.g., gag, e.g., a viral capsid (CA) protein), or a nucleic acid molecule encoding the structural polypeptide domain;
c) a reverse transcriptase polypeptide domain (e.g., pol, e.g., as listed in Table 11 or 12) capable of reverse transcribing the template RNA, thereby producing a template DNA, or a nucleic acid molecule encoding the reverse transcriptase polypeptide domain;
wherein b) and c) together are integration-deficient; and
d) a serine recombinase (e.g., serine integrase) polypeptide domain that binds the DNA recognition sequence and comprises an amino acid sequence according to any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto, or a nucleic acid molecule encoding the serine recombinase polypeptide domain,
wherein b), c), and d), are optionally part of the same polypeptide.

16. A cell-free system for modifying DNA comprising:
a) a template RNA comprising a first LTR, a second LTR, and a heterologous object sequence encoding a therapeutic effector, positioned between the first LTR and the second LTR, a DNA recognition sequence, and optionally a primer binding site (PBS); or a DNA molecule encoding the template RNA;
b) a first RNA encoding a retroviral structural polypeptide domain (e.g., gag);
c) a second RNA encoding a retroviral reverse transcriptase polypeptide domain (e.g., pol, e.g., as listed in Table 11 or 12) capable of reverse transcribing the template RNA, thereby producing a template DNA, or a nucleic acid molecule encoding the reverse transcriptase polypeptide domain;
wherein the first RNA sequence and the second RNA sequence are optionally part of the same nucleic acid molecule; and
wherein the retroviral structural polypeptide domain and the retroviral reverse transcriptase polypeptide domain together are integration-deficient; and
d) a serine recombinase (e.g., serine integrase) polypeptide domain that is exogenous to b) and c) and binds the DNA recognition sequence and is capable of integrating the template DNA into a target DNA, or a nucleic acid molecule encoding the serine recombinase polypeptide domain,
wherein b), c), and d), are optionally part of the same polypeptide.

17. The system of any of the preceding embodiments, wherein the DNA recognition sequence comprises a sequence having 30-70 or 40-60 contiguous nucleotides of SEQ ID NO: (n+13,000), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

18. The system of any of the preceding embodiments, wherein the DNA recognition sequence comprises a sequence having a first parapalindromic sequence and a second parapalindromic sequence, wherein each parapalindromic sequence is about 15-35 or 20-30 nucleotides, and the first and second parapalindromic sequences together comprise a parapalindromic region occurring within a nucleotide sequence according to SEQ ID NO: (n+13,000), or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

19. The system of any of the preceding embodiments, wherein:
the serine recombinase (e.g., serine integrase) polypeptide domain comprises the amino acid sequence in the sequence listing designated as Integrase By, wherein y is chosen from any of 2-11,258, or an amino acid sequence having at least 70%, 75%, 80%, 85%, 90%, 95%, 96%, 97%, 98%, or 99% identity thereto; and
the DNA recognition sequence comprises a sequence having 30-70 or 40-60 contiguous nucleotides of the sequence in the sequence listing designated as LeftRegion for integrase By), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

20. The system of any of the preceding embodiments, wherein:
the serine recombinase (e.g., serine integrase) polypeptide domain comprises the amino acid sequence in the sequence listing designated as Integrase By, wherein y is chosen from any of 2-11,258, or an amino acid sequence having at least 70%, 75%, 80%, 85%, 90%, 95%, 96%, 97%, 98%, or 99% identity thereto; and
the DNA recognition sequence comprises a sequence having a first parapalindromic sequence and a second parapalindromic sequence, wherein each parapalindromic sequence is about 15-35 or 20-30 nucleotides, and the first and second parapalindromic sequences together comprise a parapalindromic region occurring within a nucleotide sequence of the sequence in the sequence listing designated as LeftRegion for integrase By, or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

21. The system of any of the preceding embodiments, wherein:
the serine recombinase (e.g., serine integrase) polypeptide domain comprises the amino acid sequence in the sequence listing designated as Integrase Cy, wherein y is chosen from any of 1-175, or an amino acid sequence having at least 70%, 75%, 80%, 85%, 90%, 95%, 96%, 97%, 98%, or 99% identity thereto; and
the DNA recognition sequence comprises a sequence having 30-70 or 40-60 contiguous nucleotides of the sequence in the sequence listing designated as LeftRegion for integrase Cy), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

22. The system of any of the preceding embodiments, wherein:
the serine recombinase (e.g., serine integrase) polypeptide domain comprises the amino acid sequence in the sequence listing designated as Integrase Cy, wherein y is chosen from any of 1-175, or an amino acid sequence having at least 70%, 75%, 80%, 85%, 90%, 95%, 96%, 97%, 98%, or 99% identity thereto; and
the DNA recognition sequence comprises a sequence having a first parapalindromic sequence and a second parapalindromic sequence, wherein each parapalindromic sequence is about 15-35 or 20-30 nucleotides, and the first and second parapalindromic sequences together comprise a parapalindromic region occurring within a nucleotide sequence of the sequence in the sequence listing designated as LeftRegion for integrase Cy, or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

23. The system of any of the preceding embodiments, wherein:
the serine recombinase (e.g., serine integrase) polypeptide domain comprises an amino acid sequence of SEQ ID NO: n, wherein n is chosen from any of 1-12,677 (e.g., any of 1-11,432), or an amino acid sequence having at least 70%, 75%, 80%, 85%, 90%, 95%, 96%, 97%, 98%, or 99% identity thereto; and
the DNA recognition sequence comprises a sequence having 30-70 or 40-60 contiguous nucleotides of SEQ ID NO: (n+26,000), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

24. The system of any of the preceding embodiments, wherein:
the serine recombinase (e.g., serine integrase) polypeptide domain comprises an amino acid sequence of SEQ ID NO: n, wherein n is chosen from any of 1-12,677 (e.g., any of 1-11,432), or an amino acid sequence having at least 70%, 75%, 80%, 85%, 90%, 95%, 96%, 97%, 98%, or 99% identity thereto; and
the DNA recognition sequence comprises a sequence having a first parapalindromic sequence and a second parapalindromic sequence, wherein each parapalindromic sequence is about 15-35 or 20-30 nucleotides, and the first and second parapalindromic sequences together comprise a parapalindromic region occurring within a nucleotide sequence according to SEQ ID NO: (n+26,000), or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

25. The system of any of the preceding embodiments, wherein:
the serine recombinase (e.g., serine integrase) polypeptide domain comprises the amino acid sequence in the sequence listing designated as Integrase By, wherein y is chosen from any of 2-11,258, or an amino acid sequence having at least 70%, 75%, 80%, 85%, 90%, 95%, 96%, 97%, 98%, or 99% identity thereto; and
the DNA recognition sequence comprises a sequence having 30-70 or 40-60 contiguous nucleotides of the sequence in the sequence listing designated as RightRegion for integrase By), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

26. The system of any of the preceding embodiments, wherein:
the serine recombinase (e.g., serine integrase) polypeptide domain comprises the amino acid sequence in the sequence listing designated as Integrase By, wherein y is chosen from any of 2-11,258, or an amino acid sequence having at least 70%, 75%, 80%, 85%, 90%, 95%, 96%, 97%, 98%, or 99% identity thereto; and
the DNA recognition sequence comprises a sequence having a first parapalindromic sequence and a second parapalindromic sequence, wherein each parapalindromic sequence is about 15-35 or 20-30 nucleotides, and the first and second parapalindromic sequences together comprise a parapalindromic region occurring within a nucleotide sequence of the sequence in the sequence listing designated as RightRegion for integrase By, or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

27. The system of any of the preceding embodiments, wherein:
the serine recombinase (e.g., serine integrase) polypeptide domain comprises the amino acid sequence in the sequence listing designated as Integrase Cy, wherein y is chosen from any of 1-175, or an amino acid sequence having at least 70%, 75%, 80%, 85%, 90%, 95%, 96%, 97%, 98%, or 99% identity thereto; and
the DNA recognition sequence comprises a sequence having 30-70 or 40-60 contiguous nucleotides of the sequence in the sequence listing designated as RightRegion for integrase Cy), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

28. The system of any of the preceding embodiments, wherein:
the serine recombinase (e.g., serine integrase) polypeptide domain comprises the amino acid sequence in the sequence listing designated as Integrase Cy, wherein y is chosen from any of 1-175, or an amino acid sequence having at least 70%, 75%, 80%, 85%, 90%, 95%, 96%, 97%, 98%, or 99% identity thereto; and
the DNA recognition sequence comprises a sequence having a first parapalindromic sequence and a second parapalindromic sequence, wherein each parapalindromic sequence is about 15-35 or 20-30 nucleotides, and the first and second parapalindromic sequences together comprise a parapalindromic region occurring within a nucleotide sequence of the sequence in the sequence listing designated as RightRegion for integrase Cy, or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

29. The system of any of the preceding embodiments, wherein the lentiviral vector fuses to the target cell, the template RNA is reverse transcribed, the serine recombinase polypeptide domain is cleaved from the structural polypeptide domain by a protease (e.g., a retroviral protease, e.g., a lentiviral protease), the template DNA is circularized, and the template DNA is integrated into the genome by the serine recombinase polypeptide domain.

31. The system of any of the preceding embodiments, wherein the lentiviral vector fuses to the target cell, the template RNA is reverse transcribed, the serine recombinase polypeptide domain is cleaved from the structural polypeptide domain by a protease (e.g., a retroviral protease, e.g., a lentiviral protease), the template DNA is not circularized, and the template DNA is integrated into the genome by the serine recombinase polypeptide domain.

32. The system of any of the preceding embodiments, wherein the LTR sequences undergo homologous recombination resulting in circularization, e.g., by a host function or by a function provided by the retroviral system (e.g., overexpression of RecA).

33. The system of any of the preceding embodiments, wherein the template DNA comprises DNA recognition sequences in one or more of the LTRs (e.g., DNA recognition sequences that bind to FLP recombinase (e.g., FRT sites) or Cre recombinase (e.g., loxP sites)).

34. The system of any of the preceding embodiments, wherein the template DNA comprises a sequence that can be bound by a recombination directionality factor (RDF).

35. The system of any of the preceding embodiments, wherein the template DNA does not comprise a sequence that can be bound by a recombination directionality factor (RDF).

36. The system of any of the preceding embodiments, wherein the template RNA comprises one or more meganuclease sites (e.g., within one or more of the LTRs), e.g., an LAGLIDADG family endonuclease, e.g., I-SceI or I-CreI.

37. A fusion protein comprising:
one or both of a) a retroviral (e.g., lentiviral) structural polypeptide domain (e.g., gag), and b) a retroviral (e.g., lentiviral) reverse transcriptase polypeptide domain (e.g., pol, e.g., as listed in Table 11 or 12); and
c) serine recombinase (e.g., serine integrase) polypeptide domain.

38. The fusion protein of embodiment 37, wherein the serine recombinase polypeptide domain comprises an amino acid sequence of any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto.

39. The fusion protein of embodiment 37 or 38, wherein the retroviral (e.g., lentiviral) reverse transcriptase polypeptide domain is substantially unable to integrate the template DNA into a DNA.

40. A template RNA comprising:
a) a region comprising a DNA recognition sequence that is recognized by a serine recombinase (e.g., serine integrase) polypeptide domain;
b) a retroviral (e.g., lentiviral) attachment site;
c) heterologous object sequence encoding a therapeutic effector (e.g., wherein the therapeutic effector comprising a polypeptide or functional nucleic acid molecule, e.g., an siRNA or miRNA).

41. The template RNA of embodiment 40, wherein the serine recombinase polypeptide domain that comprises an amino acid sequence of any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto.

42. A template RNA comprising:
a) a region comprising a DNA recognition sequence that is recognized by a serine recombinase (e.g., serine integrase) polypeptide domain that comprises an amino acid sequence of any of SEQ ID NOs: 1-12,677 (e.g., any of SEQ ID NOs: 1-11,432), or an amino acid sequence having at least 70%, 75%, 80%, 85%, 90%, 95%, 96%, 97%, 98%, or 99% identity thereto, and
b) a retroviral (e.g., lentiviral) attachment site.

43. The template RNA of embodiment 42, which further comprises:
c) heterologous object sequence encoding a therapeutic effector (e.g., wherein the therapeutic effector comprising a polypeptide or functional nucleic acid molecule, e.g., an siRNA or miRNA).

44. The template RNA of any of embodiments 40-43, which comprises two retroviral (e.g., lentiviral) attachment sites (e.g., wherein each retroviral (e.g., lentiviral) attachment site is a retrovirus (e.g., lentivirus) LTR).

45. The template RNA of embodiment 44, wherein one of the retroviral (e.g., lentiviral) attachment sites is present at each end of the template RNA.

46. The template RNA of embodiment 44 or 45, wherein the LTR is a self-inactivating (SIN) LTR.

47. The template RNA of any of embodiments 40-45, which is linear.

48. A template RNA comprising a DNA recognition site specifically bound by a serine integrase (e.g., as described herein);
wherein the serine integrase is not phiC31 integrase or bxbi integrase.

49. A vector (e.g., a DNA vector) encoding the template RNA of any of embodiments 44-48.

50. A method of modifying the genome of a cell (e.g., a eukaryotic cell, e.g., a mammalian cell, e.g., human cell) comprising contacting the cell with:
a system of any of the preceding embodiments,
thereby modifying the genome of the cell.

51. The system, fusion protein, or method of any of the preceding embodiments, wherein the target DNA is a genomic DNA (e.g., a chromosome or a mitochondrial DNA), e.g., human genomic DNA.

52. The system, fusion protein, or method of any of the preceding embodiments, wherein the retroviral (e.g., lentiviral) reverse transcriptase polypeptide domain has reduced integrase activity, e.g., to at least 10%, 5%, 2%, or 1% of that of a corresponding wild-type sequence, e.g., as measured in an assay as described in Moldt et al. 2008 (BMC Biotechnol. 8:60; incorporated herein by reference).

53. The system, fusion protein, or method of any of the preceding embodiments, wherein the retroviral (e.g., lentiviral) reverse transcriptase polypeptide domain comprises a mutation that reduces integrase activity, e.g., to no more than about 75%, 50%, 40%, 30%, 25%, 20%, 10%, 5%, 2%, or 1% of a corresponding wild-type sequence, e.g., as measured in an assay as described in Moldt et al. 2008 (BMC Biotechnol. 8:60).

54. The system, fusion protein, or method of any of the preceding embodiments, wherein the system does not comprise a wild-type retroviral (e.g., lentiviral) integrase.

55. The system, fusion protein, or method of any of the preceding embodiments, wherein the system comprises a mutated retroviral (e.g., lentiviral) integrase.

56. The system, fusion protein, or method of any of the preceding embodiments, wherein the retroviral (e.g., lentiviral) reverse transcriptase polypeptide domain comprises a mutated retroviral (e.g., lentiviral) integrase.

57. The system, fusion protein, or method of any of the preceding embodiments, wherein the template RNA comprises a nucleic acid sequence encoding a mutated retroviral (e.g., lentiviral) integrase.

58. The system, fusion protein, or method of any of embodiments 55-57, wherein the mutated retroviral (e.g., lentiviral) integrase comprises at least one amino acid difference relative to a wild-type retroviral (e.g., lentiviral) integrase.

59. The system, fusion protein, or method of any of embodiments 55-58, wherein the mutated retroviral (e.g., lentiviral) integrase comprises a substitution, addition, or deletion relative to a wild-type retroviral (e.g., lentiviral) integrase.

60. The system, fusion protein, or method of any of embodiments 55-59, wherein the mutated retroviral (e.g., lentiviral) integrase has less than 10%, 9%, 8%, 7%, 6%, 5%, 4%, 3%, 2%, or 1% of the activity of the wild-type integrase.

61. The system, fusion protein, or method of any of embodiments 53-60, wherein the mutation is a class I mutation (e.g., as described in Wanisch et al. 2009, Mol. Therap. 17(8): 1316-1332).

62. The system, fusion protein, or method of any of embodiments 53-61, wherein the mutation comprises a mutation in a catalytic triad residue (e.g., mutations in 1, 2, or 3 catalytic triad residues).

63. The system, fusion protein, or method of any of embodiments 53-62, wherein the mutation comprises a substitution at D64 (e.g., D64V), D116, and/or E152 of the amino acid sequence of an HIV-1 integrase (IN) protein.

64. The system, fusion protein, or method of any of embodiments 53-63, wherein the mutation comprises a substitution at one or more of the following residues: H12, D64, D64, D64, D116, N120, Q148, F185, W235, R262, R263, K264, K264, K264, K266, and/or K273.

65. The system, fusion protein, or method of any of embodiments 53-64, wherein the mutation comprises one or more of the following substitutions: H12A, D64V, D64A, D64E, D116N, N120L, Q148A, F185A, W235E, R262A, R263A, K264H, K264R, K264E, K266R, and/or K273R.

66. The system, fusion protein, or method of any of embodiments 53-65, wherein the mutation comprises the substitution D64V.

67. The system, fusion protein, or method of any of embodiments 53-66, wherein the mutation comprises the following substitutions: K264R, K266R, and K273R.

68. The system, fusion protein, or method of any of embodiments 53-67, wherein the mutation comprises the following substitutions: D64V and N120L.

69. The system, fusion protein, or method of any of embodiments 53-68, wherein the mutation comprises the following substitutions: D64V and W235E.

70. The system, fusion protein, or method of any of embodiments 53-69, wherein the mutation comprises the following substitutions: D64V, N120L, and W235E.

71. The system, fusion protein, or method of any of embodiments 53-70, wherein the mutation comprises the following substitutions: R262A, R263A, and K264H

72. The system, fusion protein, or method of any of embodiments 53-71, wherein the mutation comprises the following substitutions: K264E, F185A, D116A, D64A, and H12A.

73. The system, fusion protein, or method of any of embodiments 53-72, wherein the mutation comprises the following substitutions: D64N and D116N.

74. The system, fusion protein, or method of any of embodiments 53-73, wherein the mutation is a class II mutation.

75. The system or method of any of the preceding embodiments, wherein the system further comprises, or wherein the method further comprises contacting the cell with, an inhibitor of integrase activity of (c).

76. The system, fusion protein, or method of embodiment 75, wherein the inhibitor of integrase activity is an inhibitor of a retroviral (e.g., lentiviral) integrase protein (e.g., an HIV integrase protein).

77. The system, fusion protein, or method of embodiment 75 or 76, wherein the inhibitor of integrase activity reduces the integrase activity of a retroviral (e.g., lentiviral) integrase protein (e.g., an HIV integrase protein) by at least 20%, 30%, 40%, 50%, 60%, 70%, 75%, 80%, 85%, 90%, 95%, 96%, 97%, 98%, or 99%.

78. The system or method of any of embodiments 75-77, wherein the inhibitor is a small molecule.

79. The system, fusion protein, or method of any of embodiments 75-78, wherein the inhibitor is a strand-transfer inhibitor.

80. The system, fusion protein, or method of any of embodiments 75-79, wherein the inhibitor is raltegravir or elvitegravir, or a salt thereof.

81. The system, fusion protein, or method of any of embodiments 75-80, wherein the inhibitor is an inhibitor of binding between a retroviral (e.g., lentiviral) integrase and a cellular cofactor.

82. The system, fusion protein, or method of any of embodiments 75-81, wherein the cellular cofactor is LEDGF/p75, integrase interactor 1, gemin2, emerin, or barrier to autointegration factor (BAF).

83. The system, fusion protein, or method of any of the preceding embodiments, wherein the template RNA comprises a retroviral (e.g., lentiviral) attachment site, e.g., at one end of the template RNA.

84. The system, fusion protein, or method of any of the preceding embodiments wherein the template RNA comprises two retroviral (e.g., lentiviral) attachment sites, e.g., one at each end of the template RNA.

85. The system, fusion protein, or method of any of the preceding embodiments, wherein the template RNA is packaged by the retroviral (e.g., lentiviral) structural polypeptide domain (e.g., gag).

86. The system, fusion protein, or method of any of the preceding embodiments, wherein the template RNA does not comprise a wild-type retroviral (e.g., lentiviral) attachment site at one or both ends.

87. The system, fusion protein, or method of any of the preceding embodiments, wherein the template RNA does not comprise a retroviral (e.g., lentiviral) attachment site that differs from a wild-type retroviral (e.g., lentiviral) attachment site only by one or more self-inactivating mutations, e.g., at one or both ends.

88. The system, fusion protein, or method of any of the preceding embodiments, wherein the template RNA does not comprise a wild-type retroviral (e.g., lentiviral) attachment site at its 5′ end.

89. The system, fusion protein, or method of any of the preceding embodiments, wherein the template RNA does not comprise a wild-type retroviral (e.g., lentiviral) attachment site at its 3′ end.

90. The system, fusion protein, or method of any of the preceding embodiments, wherein the template RNA comprises one or more (e.g., 1 or 2) mutated retroviral (e.g., lentiviral) attachment sites (e.g., comprising a nucleic acid sequence comprising at least one addition, deletion, or substitution relative to the sequence of a wild-type retroviral (e.g., lentiviral) attachment site).

91. The system, fusion protein, or method of any of the preceding embodiments, wherein the template comprises a mutated retroviral (e.g., lentiviral) attachment site in a U3 region.

92. The system, fusion protein, or method of any of the preceding embodiments, wherein the template comprises a mutated retroviral (e.g., lentiviral) attachment site in a U5 region.

93. The system, fusion protein, or method of any of the preceding embodiments, wherein the template comprises a first mutated retroviral (e.g., lentiviral) attachment site in a U3 region and second mutated retroviral (e.g., lentiviral) attachment site in a U5 region (e.g., wherein the first and second mutated retroviral (e.g., lentiviral) attachment sites have the same sequence, or wherein the first and second mutated retroviral (e.g., lentiviral) attachment sites have different sequences).

94. The system, fusion protein, or method of any of the preceding embodiments, wherein the wild-type retroviral (e.g., lentiviral) attachment site is a wild-type HIV (e.g., HIV-1 or HIV-2) attachment site.

95. The system, fusion protein, or method of any of the preceding embodiments, wherein the wild-type retroviral (e.g., lentiviral) attachment site comprises a long terminal repeat (LTR), e.g., an LTR having the sequence of:

| | |
|---|---|
| (i) | GGGTCTCTCTGGTTAGACCAGATCTGAGCCTGGGAGCTCTCTGG |
| | CTAACTAGGGAACCCACTGCTTAAGCCTCAATAAAGCTTGCCTT |
| | GAGTGCTTCAAGTAGTGTGTGCCCGTCTGTTGTGTGACTCTGGT |
| | AACTAGAGATCCCTCAGACCCTTTTAGTCAGTGTGGAAAATCTC |
| | TAGCA, |
| (ii) | GGGTCTCTCTGGTTAGACCAGATCTGAGCCTGGGAGCTCTCTGG |
| | CTAACTAGGGAACCCACTGCTTAAGCCTCAATAAAGCTTGCCTT |
| | GAGTGCTTCAAGTAGTGTGTGCCCGTCTGTTGTGTGACTCTGGT |
| | AACTAGAGATCCCTCAGACCCTTTTAGTCAGTGTGGAAAATCTC |
| | TAGCA, |
| iii) | GGGTCTCTCTGGTTAGACCAGATCTGAGCCTGGGAGCTCTCTGG |
| | CTAACTAGGGAACCCACTGCTTAAGCCTCAATAAAGCTTGCCTT |
| | GAGTGCTTCAAGTAGTGTGTGCCCGTCTGTTGTGTGACTCTGGT |
| | AACTAGAGATCCCTCAGACCCTTTTAGTCAGTGTGGAAAATCTC |
| | TAGCA, |
| | or |
| (iv) | TGGAAGGGCTAATTCACTCCCAACGAAGACAAGATCTGCTTTTT |
| | GCTTGTACTGGGTCTCTCTGGTTAGACCAGATCTGAGCCTGGGA |
| | GCTCTCTGGCTAACTAGGGAACCCACTGCTTAAGCCTCAATAAA |
| | GCTTGCCTTGAGTGCTTCAAGTAGTGTGTGCCCGTCTGTTGTGT |
| | GACTCTGGTAACTAGAGATCCCTCAGACCCTTTTAGTCAGTGTG |
| | GAAAATCTCTAGCA; |

a sequence having at least 75%, 80%, 85%, 90%, 95%, 96%, 97%, 98%, or 99% sequence identity thereto.

96. The system, fusion protein, or method of embodiment 95, wherein the LTR is a self-inactivating (SIN) LTR.

97. The system, fusion protein, or method of any of the preceding embodiments, wherein the template RNA does not comprise a wild-type LTR sequence from a retrovirus (e.g., lentivirus) (e.g., HIV, e.g., HIV-1 or HIV-2).

98. The system, fusion protein, or method of any of the preceding embodiments, wherein the template RNA comprises a mutated LTR sequence (e.g., an LTR sequence comprising at least one nucleotide difference (e.g., an addition, substitution, or deletion) from a wild-type retroviral (e.g., lentiviral) LTR sequence)

99. The system, fusion protein, or method of embodiment 98, wherein the mutation does not substantially reduce reverse transcriptase activity, e.g., wherein reverse transcriptase activity is 80%-100% of that of a corresponding wild-type sequence.

100. The system, fusion protein, or method of any of the preceding embodiments, wherein the nucleic acid molecule encoding the retroviral (e.g., lentiviral) structural polypeptide domain and the retroviral (e.g., lentiviral) reverse transcriptase polypeptide domain does not comprise a nucleic acid sequence encoding a retroviral (e.g., lentiviral) vif, vpr, vpu, and/or nef protein.

101. The system, fusion protein, or method of any of the preceding embodiments, wherein the nucleic acid molecule encoding the retroviral (e.g., lentiviral) structural polypeptide domain and the retroviral (e.g., lentiviral) reverse transcriptase polypeptide domain comprises a nucleic acid sequence encoding a retroviral (e.g., lentiviral) vif, vpr, vpu, and/or nef protein.

102. The system, fusion protein, or method of any of the preceding embodiments, wherein the nucleic acid molecule encoding the retroviral (e.g., lentiviral) structural polypeptide domain and the retroviral (e.g., lentiviral) reverse transcriptase polypeptide domain does not comprise a nucleic acid sequence encoding a retroviral (e.g., lentiviral) tat protein.

103. The system, fusion protein, or method of any of the preceding embodiments, wherein the system does not comprise a retroviral (e.g., lentiviral) vif, vpr, vpu, and/or nef protein, and/or a nucleic acid sequence encoding the retroviral (e.g., lentiviral) vif, vpr, vpu, and/or nef protein.

104. The system, fusion protein, or method of any of the preceding embodiments, wherein the system does not comprise a retroviral (e.g., lentiviral) tat protein, and/or a nucleic acid sequence encoding the retroviral (e.g., lentiviral) tat protein.

105. The system, fusion protein, or method of any of the preceding embodiments, wherein the template RNA comprises one or more (e.g., 1, 2, 3, or all 4) of:
(a) a polynucleotide encoding a protein binding sequence (PBS), e.g., of a retrovirus (e.g., a lentivirus), or a nucleic acid sequence having at least 75%, 80%, 85%, 90%, 95%, 96%, 97%, 98%, or 99% sequence identity thereto;
(b) a polynucleotide encoding a polypurine tract (PPT), e.g., of a retrovirus (e.g., a lentivirus), or a nucleic acid sequence having at least 75%, 80%, 85%, 90%, 95%, 96%, 97%, 98%, or 99% sequence identity thereto;
(c) a polynucleotide encoding a retroviral (e.g., lentiviral) Psi packaging element, or a nucleic acid sequence having at least 75%, 80%, 85%, 90%, 95%, 96%, 97%, 98%, or 99% sequence identity thereto; and/or
(d) a polynucleotide encoding a dimer initiation site (DIS), e.g., of a retrovirus (e.g., a lentivirus), or a nucleic acid sequence having at least 75%, 80%, 85%, 90%, 95%, 96%, 97%, 98%, or 99% sequence identity thereto.

106. The system, fusion protein, or method of any of the preceding embodiments, wherein the template RNA comprises one or more (e.g., 1, 2, 3, 4, 5, 6, 7, 8, 9, 10, or all 11) of:
(i) one or more long terminal repeats (LTR) (e.g., one or two LTRs, e.g., positioned at the 5′ and/or 3′ ends of the template RNA); optionally wherein one or more of the LTRs are self-inactivated LTRs,
(ii) a gag-encoding sequence (e.g., a gene encoding a polypeptide having at least 75%, 80%, 85%, 90%, 95%, 96%, 97%, 98%, 99%, or 100% amino acid sequence identity to the gag protein of a retrovirus (e.g., lentivirus)),
(iii) a pol-encoding sequence (e.g., a gene encoding a polypeptide having at least 75%, 80%, 85%, 90%, 95%, 96%, 97%, 98%, 99%, or 100% amino acid sequence identity to the pol protein of a retrovirus (e.g., lentivirus)),
(iv) a vif-encoding sequence (e.g., a gene encoding a polypeptide having at least 75%, 80%, 85%, 90%, 95%, 96%, 97%, 98%, 99%, or 100% amino acid sequence identity to the vif protein of a retrovirus (e.g., lentivirus)),
(v) a vpr-encoding sequence (e.g., a gene encoding a polypeptide having at least 75%, 80%, 85%, 90%, 95%, 96%, 97%, 98%, 99%, or 100% amino acid sequence identity to the vpr protein of a retrovirus (e.g., lentivirus)),
(vi) a tat-encoding sequence (e.g., a gene encoding a polypeptide having at least 75%, 80%, 85%, 90%, 95%, 96%, 97%, 98%, 99%, or 100% amino acid sequence identity to the tat protein of a retrovirus (e.g., lentivirus)),
(vii) a rev-encoding sequence (e.g., a gene encoding a polypeptide having at least 75%, 80%, 85%, 90%, 95%, 96%, 97%, 98%, 99%, or 100% amino acid sequence identity to the rev protein of a retrovirus (e.g., lentivirus)),
(viii) a vpu-encoding sequence (e.g., a gene encoding a polypeptide having at least 75%, 80%, 85%, 90%, 95%, 96%, 97%, 98%, 99%, or 100% amino acid sequence identity to the vpu protein of a retrovirus (e.g., lentivirus)),
(ix) a gp120-encoding sequence (e.g., a gene encoding a polypeptide having at least 75%, 80%, 85%, 90%, 95%, 96%, 97%, 98%, 99%, or 100% amino acid sequence identity to the gp120 protein of a retrovirus (e.g., lentivirus)),
(x) a gp41-encoding sequence (e.g., a gene encoding a polypeptide having at least 75%, 80%, 85%, 90%, 95%, 96%, 97%, 98%, 99%, or 100% amino acid sequence identity to the gp41 protein of a retrovirus (e.g., lentivirus)), and/or
(xi) a nef-encoding sequence (e.g., a gene encoding a polypeptide having at least 75%, 80%, 85%, 90%, 95%, 96%, 97%, 98%, 99%, or 100% amino acid sequence identity to the nef protein of a retrovirus (e.g., lentivirus)).

107. The system, fusion protein, or method of any of the preceding embodiments, wherein the system further comprises one or more nucleic acid molecules (e.g., a vector, e.g., a packaging vector) comprising one or more (e.g., 1, 2, 3, 4, 5, 6, 7, 8, 9, 10, or all 11) of:
(i) one or more long terminal repeats (LTR) (e.g., one or two LTRs, e.g., positioned at the 5′ and/or 3′ ends of the template RNA); optionally wherein one or more of the LTRs are self-inactivated LTRs,
(ii) a gag-encoding sequence (e.g., a gene encoding a polypeptide having at least 75%, 80%, 85%, 90%, 95%, 96%, 97%, 98%, 99%, or 100% amino acid sequence identity to the gag protein of a retrovirus (e.g., lentivirus)),
(iii) a pol-encoding sequence (e.g., a gene encoding a polypeptide having at least 75%, 80%, 85%, 90%, 95%, 96%, 97%, 98%, 99%, or 100% amino acid sequence identity to the pol protein of a retrovirus (e.g., lentivirus)),
(iv) a vif-encoding sequence (e.g., a gene encoding a polypeptide having at least 75%, 80%, 85%, 90%, 95%, 96%, 97%, 98%, 99%, or 100% amino acid sequence identity to the vif protein of a retrovirus (e.g., lentivirus)),
(v) a vpr-encoding sequence (e.g., a gene encoding a polypeptide having at least 75%, 80%, 85%, 90%, 95%, 96%, 97%, 98%, 99%, or 100% amino acid sequence identity to the vpr protein of a retrovirus (e.g., lentivirus)),
(vi) a tat-encoding sequence (e.g., a gene encoding a polypeptide having at least 75%, 80%, 85%, 90%, 95%, 96%, 97%, 98%, 99%, or 100% amino acid sequence identity to the tat protein of a retrovirus (e.g., lentivirus)),
(vii) a rev-encoding sequence (e.g., a gene encoding a polypeptide having at least 75%, 80%, 85%, 90%, 95%, 96%, 97%, 98%, 99%, or 100% amino acid sequence identity to the rev protein of a retrovirus (e.g., lentivirus)),
(viii) a vpu-encoding sequence (e.g., a gene encoding a polypeptide having at least 75%, 80%, 85%, 90%, 95%, 96%, 97%, 98%, 99%, or 100% amino acid sequence identity to the vpu protein of a retrovirus (e.g., lentivirus)),
(ix) a gp120-encoding sequence (e.g., a gene encoding a polypeptide having at least 75%, 80%, 85%, 90%, 95%, 96%, 97%, 98%, 99%, or 100% amino acid sequence identity to the gp120 protein of a retrovirus (e.g., lentivirus)),
(x) a gp41-encoding sequence (e.g., a gene encoding a polypeptide having at least 75%, 80%, 85%, 90%, 95%, 96%, 97%, 98%, 99%, or 100% amino acid sequence identity to the gp41 protein of a retrovirus (e.g., lentivirus)), and/or
(xi) a nef-encoding sequence (e.g., a gene encoding a polypeptide having at least 75%, 80%, 85%, 90%, 95%, 96%, 97%, 98%, 99%, or 100% amino acid sequence identity to the nef protein of a retrovirus (e.g., lentivirus)).

108. The system, fusion protein, or method of embodiment 106 or 107, wherein the retrovirus (e.g., lentivirus) of any of (ii)-(xi) is an HIV (e.g., HIV-1 or HIV-2).

109. The system, fusion protein, or method of any of the preceding embodiments, wherein the template RNA comprises (e.g., in a pol-encoding gene) a retrovirus (e.g., lentivirus) integrase (IN)-encoding gene (e.g., a gene encoding a polypeptide having at least 75%, 80%, 85%, 90%, 95%, 96%, 97%, 98%, 99%, or 100% amino acid sequence identity to the IN protein of a retrovirus (e.g., lentivirus)).

110. The system, fusion protein, or method of any of the preceding embodiments, wherein the template RNA does not comprise a retrovirus (e.g., lentivirus) integrase (IN)-encoding gene (e.g., a gene encoding a polypeptide having at least 75%, 80%, 85%, 90%, 95%, 96%, 97%, 98%, 99%, or 100% amino acid sequence identity to the IN protein of a retrovirus (e.g., lentivirus)).

111. The system, fusion protein, or method of any of the preceding embodiments, wherein the retrovirus (e.g., lentivirus) is an HIV (e.g., HIV-1 or HIV-2).

112. The system, fusion protein, or method of any of the preceding embodiments, wherein the gag-encoding gene further encodes the serine recombinase polypeptide domain.

113. The system, fusion protein, or method of any of the preceding embodiments, wherein the pol-encoding gene further encodes the serine recombinase polypeptide domain.

114. The system, fusion protein, or method of any of the preceding embodiments, wherein the template RNA encodes one DNA recognition sequence.

115. The system, fusion protein, or method of any of the preceding embodiments, wherein the template RNA encodes more than one (e.g., two) DNA recognition sequences.

116. The system, fusion protein, or method of any of the preceding embodiments, wherein the template RNA is a single-stranded RNA.

117. The system, fusion protein, or method of any of the preceding embodiments, wherein the template DNA is a double-stranded DNA.

118. The system, fusion protein, or method of any of the preceding embodiments, wherein the template DNA is a single-stranded DNA.

119. The system, fusion protein, or method of any of the preceding embodiments, wherein the template RNA comprises a heterologous objection sequence.

120. The system, fusion protein, or method of any of the preceding embodiments, wherein the sequence encoding the DNA recognition sequence is within 1, 2, 3, 4, 5, 6, 7, 8, 9, 10, 15, 20, 25, 30, 40, 50, 60, 70, 80, 90, 100, 200, 300, 400, 500, 600, 700, 800, 900, 1000, 1500, 2000, 2500, 3000, 3500, 4000, 4500, 5000 nucleotides, or more, of the heterologous object sequence.

121. The system, fusion protein, or method of embodiment 119 or 120, wherein the serine recombinase polypeptide domain is capable of integrating the heterologous object sequence into the target DNA.

122. The system, fusion protein, or method of embodiment 121, wherein the heterologous object sequence is inserted into the genome of the cell at an efficiency of at least about 0.1% (e.g., at least about 0.1%, 0.5%, 1%, 5%, 10%, 20%, 30%, 40%, 50%, 60%, 70%, 80%, 85%, 90%, 95%, 96%, 97%, 98%, 99%, or 100%) of a population of the cell, e.g., as measured in an assay of Example 31 or 33.

123. The system, fusion protein, or method of embodiment 121 or 122, wherein the heterologous object sequence is inserted into a site within the genome of the cell (e.g., a cognate DNA recognition sequence bound by a recombinase that binds to a DNA recognition sequence occurring within the template RNA: comprising a sequence of SEQ ID NO: (n+13,000) or a sequence of SEQ ID NO: (n+26,000), wherein n is chosen from any of 1-12,677 (e.g., any of 1-11,432) (e.g., a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432)), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto; and/or a recombinase comprising a corresponding amino acid sequence of SEQ ID NO: n) in at least about 1%, (e.g., at least about 1%, 5%, 10%, 15%, 20%, 25%, 30%, 40%, 50%, 60%, 70%, 80%, 85%, 90%, 95%, 96%, 97%, 98%, 99%, 99.9%, or 100%) of insertion events.

124. The system, fusion protein, or method of any of embodiments 121-123, wherein, in a population of the cells (e.g., contacted with the system), the heterologous object sequence is inserted into between 1-10, e.g., 1-9, 1-8, 1-7, 1-6, 1-5, 1-4, 1-3, 2-10, 2-5, 2-4, 3-10, 3-5, or 5-10 sites within the genome of the cell (e.g., a cognate DNA recognition sequence bound by a recombinase that binds to a DNA recognition sequence occurring within the template RNA: comprising a sequence of SEQ ID NO: (n+13,000) or a sequence of SEQ ID NO: (n+26,000), wherein n is chosen from any of 1-12,677 (e.g., any of 1-11,432) (e.g., a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432)), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto; and/or a recombinase comprising a corresponding amino acid sequence of SEQ ID NO: n), in at least 1%, 5%, 10%, 15%, 20%, 25%, 30%, 40%, 50%, 60%, 70%, 80%, 85%, 90%, 95%, 96%, 97%, 98%, 99%, 99.9%, or 100% of the cells in the population.

125. The system, fusion protein, or method of any of embodiments 119-124, wherein the heterologous object sequence comprises a eukaryotic gene, e.g., a mammalian gene, e.g., human gene, e.g., a blood factor (e.g., genome factor I, II, V, VII, X, XI, XII or XIII) or enzyme, e.g., lysosomal enzyme, or synthetic human gene (e.g. a chimeric antigen receptor).

126. The system, fusion protein, or method of any of embodiments 119-125, wherein the heterologous object sequence comprises an enzyme, a structural protein, a signaling protein, a regulatory protein, a transport protein, a sensory protein, a motor protein, a defense protein, a storage protein, an immune receptor protein (e.g. a synthetic immune receptor protein such as a chimeric antigen receptor protein (CAR), a T cell receptor, or a B cell receptor), or an antibody.

127. The system, fusion protein, or method of any of the preceding embodiments, wherein the DNA recognition sequence comprises a first parapalindromic sequence and a second parapalindromic sequence, and a core sequence situated between the first and second parapalindromic sequences.

128. The system, fusion protein, or method of embodiment 127, wherein the template RNA comprises a heterologous object sequence disposed between the first parapalindromic sequence and the second parapalindromic sequence.

129. The system, fusion protein, or method of embodiment 127 or 128, wherein each parapalindromic sequence is about 15-35 or 20-30 nucleotides.

130. The system, fusion protein, or method of any of embodiments 127-129, wherein the first and second parapalindromic sequences together comprise a parapalindromic region occurring within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

131. The system, fusion protein, or method of any of embodiments 127-130, wherein the core sequence has a length of about 2-20 nucleotides.

132. The system, fusion protein, or method of any of the preceding embodiments, wherein the template DNA is capable of replicating in a cell.

133. The system, fusion protein, or method of any of the preceding embodiments, wherein the template DNA is circular.

134. The system, fusion protein, or method of any of the preceding embodiments, wherein the template DNA is circularized, e.g., to form an episome.

135. The system, fusion protein, or method of any of the preceding embodiments, wherein the template DNA is circularized by endogenous machinery, e.g., in a target cell.

136. The system, fusion protein, or method of any of the preceding embodiments, wherein the template DNA is circularized by nonhomologous end joining.

137. The system, fusion protein, or method of any of the preceding embodiments, wherein the template DNA is circularized by homologous recombination.

138. The system, fusion protein, or method of any of the preceding embodiments, wherein the template DNA is circularized by ligation.

139. The system, fusion protein, or method of any of the preceding embodiments, wherein the template DNA comprises one long terminal repeat (LTR).

140. The system, fusion protein, or method of any of the preceding embodiments, wherein the template DNA comprises two LTRs (e.g., two copies of the same LTR or two different LTRs).

141. The system, fusion protein, or method of embodiment 140, wherein the template DNA is linear and wherein one LTR is positioned at the 5′ end of the template DNA and the other LTR is positioned at the 3′ end of the template DNA.

142. The system, fusion protein, or method of embodiment 140 or 141, wherein the template DNA is circular and wherein the two LTRs are adjacent to each other.

143. The system, fusion protein, or method of any of the preceding embodiments, wherein the retroviral (e.g., lentiviral) structural polypeptide domain and/or the retroviral (e.g., lentiviral) reverse transcriptase domain are from an HIV (e.g., HIV-1 or HIV-2).

144. The system, fusion protein, or method of any of the preceding embodiments, wherein the retroviral (e.g., lentiviral) structural polypeptide domain and/or the retroviral (e.g., lentiviral) reverse transcriptase domain are from a retrovirus, e.g., an Orthoretrovirus (e.g., an Alpharetrovirus, Betaretrovirus, Deltaretrovirus, Epsilonretrovirus, Gammaretrovirus, or Lentivirus) or a Spumaretrovirus (e.g., Bovispumavirus, Equispumavirus, Felispumavirus, Prosimiispumavirus, or Simiispumavirus).

145. The system, fusion protein, or method of any of the preceding embodiments, wherein the retroviral (e.g., lentiviral) structural polypeptide domain and/or the retroviral (e.g., lentiviral) reverse transcriptase domain are from a retroviral replicating vector (RRV), gammaretrovirus (GRV), Moloney murine sarcoma virus (MMSV), Moloney murine leukemia virus (MoMLV), murine stem cell virus (MSCV), murine leukemia virus (MMLV), human foamy virus, murine mammary tumor virus (MMTV), human T-cell leukemia virus (HTLV), bovine leukemia virus (BLV), Avian leukosis virus (ALV), Rous sarcoma virus (RSV), FIV, SIV, caprine arthritis encephalitis virus (CAEV), equine infectious anemia virus (EIAV), or maedi/visna virus (MVV).

146. The system, fusion protein, or method of any of the preceding embodiments, wherein the retroviral (e.g., lentiviral) structural polypeptide domain and the retroviral (e.g., lentiviral) reverse transcriptase polypeptide domain are part of the same polypeptide.

147. The system, fusion protein, or method of any of the preceding embodiments, wherein the retroviral (e.g., lentiviral) structural polypeptide domain and the serine recombinase polypeptide domain are part of the same polypeptide.

148. The system, fusion protein, or method of any of the preceding embodiments, wherein the retroviral (e.g., lentiviral) reverse transcriptase polypeptide domain and the serine recombinase polypeptide domain are part of the same polypeptide.

149. The system, fusion protein, or method of any of the preceding embodiments, wherein the retroviral (e.g., lentiviral) structural polypeptide domain, the retroviral (e.g., lentiviral) reverse transcriptase polypeptide domain, and the serine recombinase polypeptide domain are part of the same polypeptide.

150. The system, fusion protein, or method of any of the preceding embodiments, wherein the retroviral (e.g., lentiviral) structural polypeptide domain and the serine recombinase polypeptide domain are separate polypeptides.

151. The system, fusion protein, or method of any of the preceding embodiments, wherein the retroviral (e.g., lentiviral) reverse transcriptase polypeptide domain and the serine recombinase polypeptide domain are separate polypeptides.

152. The system, fusion protein, or method of any of the preceding embodiments, wherein the retroviral (e.g., lentiviral) structural polypeptide domain comprises an HIV-1 gag amino acid sequence as listed in Table 11 or 12, or an amino acid sequence having at least 75%, 80%, 85%, 90%, 95%, 96%, 97%, 98%, or 99% sequence identity thereto.

153. The system, fusion protein, or method of any of the preceding embodiments, wherein the retroviral (e.g., lentiviral) reverse transcriptase polypeptide domain comprises an HIV-1 pol amino acid sequence as listed in Table 11 or 12, or an amino acid sequence having at least 75%, 80%, 85%, 90%, 95%, 96%, 97%, 98%, or 99% sequence identity thereto.

154. The system, fusion protein, or method of any of the preceding embodiments, wherein the retroviral (e.g., lentiviral) reverse transcriptase polypeptide domain comprises an HIV-1 integrase amino acid sequence as listed in Table 11 or 12, or an amino acid sequence having at least 75%, 80%, 85%, 90%, 95%, 96%, 97%, 98%, or 99% sequence identity thereto.

155. The system, fusion protein, or method of any of the preceding embodiments, wherein the retroviral (e.g., lentiviral) structural polypeptide domain and the serine recombinase polypeptide domain are connected by a linker (e.g., a cleavable linker, e.g., a linker cleavable by a protease).

156. The system, fusion protein, or method of embodiment 155, wherein the link comprises a protease recognition site.

157. The system, fusion protein, or method of embodiment 155 or 156, wherein the linker is attached to the N-terminal end of the retroviral (e.g., lentiviral) structural polypeptide domain.

158. The system, fusion protein, or method of embodiment 155 or 156, wherein the linker is attached to a retroviral (e.g., lentiviral) matrix protein.

159. The system, fusion protein, or method of any of the preceding embodiments, wherein the retroviral (e.g., lentiviral) reverse transcriptase polypeptide domain and the serine recombinase polypeptide domain are connected by a linker (e.g., a cleavable linker).

160. The system, fusion protein, or method of embodiment 155, wherein the linker is attached to the C-terminal end of the retroviral (e.g., lentiviral) structural polypeptide domain.

161. The system, fusion protein, or method of any of the preceding embodiments, wherein the system does not comprise a Flp recombinase, or a nucleic acid molecule encoding a Flp recombinase.

162. The system, fusion protein, or method of any of the preceding embodiments, wherein the template RNA, or the DNA molecule encoding the template RNA, does not comprise an FRT site.

163. The system, fusion protein, or method of any of the preceding embodiments, wherein the system does not comprise a transposase.

164. The system, fusion protein, or method of any of the preceding embodiments, wherein the system does not comprise a Sleeping Beauty transposase, or a nucleic acid molecule encoding a Sleeping Beauty transposase.

165. The system, fusion protein, or method of any of the preceding embodiments, wherein the template RNA, or the DNA molecule encoding the template RNA, does not comprise a Sleeping Beauty RIR site and/or a Sleeping Beauty LIR site.

166. The system, fusion protein, or method of any of the preceding embodiments, wherein the system does not comprise a phiC31 integrase, or a nucleic acid molecule encoding a phiC31 integrase.

167. The system, fusion protein, or method of any of the preceding embodiments, wherein the template RNA, or the DNA molecule encoding the template RNA, does not comprise an attB site recognized by a phiC31 integrase (e.g., an attB site having a nucleic acid sequence as shown in FIG. 4 of Grandchamp et al. 2014; PLOS ONE 9(6): e99649).

168. The system, fusion protein, or method of any of the preceding embodiments, wherein the template RNA, or the DNA molecule encoding the template RNA, does not comprise an attP site recognized by a phiC31 integrase (e.g., an attP site having a nucleic acid sequence as shown in FIG. 4 of Grandchamp et al. 2014; PLOS ONE 9(6): e99649).

169. The system, fusion protein, or method of any of the preceding embodiments, wherein the system does not comprise a piggyBac transposase, or a nucleic acid molecule encoding a piggyBac transposase.

170. The system, fusion protein, or method of any of the preceding embodiments, wherein the template DNA does not comprise a piggyBac transposase recognition site.

171. The system, fusion protein, or method of any of the preceding embodiments, wherein the retroviral (e.g., lentiviral) structural polypeptide domain is provided as an RNA molecule encoding the retroviral (e.g., lentiviral) structural polypeptide domain.

172. The system, fusion protein, or method of any of the preceding embodiments, wherein the retroviral (e.g., lentiviral) reverse transcriptase polypeptide domain is provided as an RNA molecule encoding the retroviral (e.g., lentiviral) reverse transcriptase polypeptide domain.

173. The system, fusion protein, or method of any of the preceding embodiments, wherein the serine recombinase polypeptide domain is provided as an RNA molecule encoding the serine recombinase polypeptide domain.

174. The system, fusion protein, or method of any of the preceding embodiments, wherein the retroviral (e.g., lentiviral) structural polypeptide domain is provided as a polypeptide (e.g., as a domain of a polypeptide).

175. The system, fusion protein, or method of any of the preceding embodiments, wherein the retroviral (e.g., lentiviral) reverse transcriptase polypeptide domain is provided as a polypeptide (e.g., as a domain of a polypeptide).

176. The system, fusion protein, or method of any of the preceding embodiments, wherein the serine recombinase polypeptide domain is provided as a polypeptide (e.g., as a domain of a polypeptide).

177. The system, fusion protein, or method of embodiment 176, wherein the serine recombinase polypeptide domain is provided in an exosome, e.g., wherein the serine recombinase polypeptide domain is fused to a domain that binds a membrane protein in the exosome.

178. The system, fusion protein, or method of embodiment 176, wherein the template RNA, structural polypeptide domain, reverse transcriptase polypeptide domain, and/or serine recombinase polypeptide domain is introduced into the cell via a nanoparticle, lipid nanoparticle, fusosome, or vesicle.

179. The system, fusion protein, or method of any of the preceding embodiments, wherein the template RNA is enclosed in a proteinaceous exterior (e.g., comprised in a retroviral (e.g., lentiviral) particle, e.g., an integration-deficient retrovirus (e.g., lentivirus)).

180. The system, fusion protein, or method of any of the preceding embodiments, wherein the serine recombinase polypeptide domain is enclosed in a proteinaceous exterior (e.g., comprised in a retroviral (e.g., lentiviral) particle, e.g., an integration-deficient retrovirus (e.g., lentivirus)).

181. The system, fusion protein, or method of any of the preceding embodiments, wherein the serine recombinase polypeptide domain is provided as an RNA encoding the serine recombinase polypeptide domain, that is not enclosed in a proteinaceous exterior.

182. The system, fusion protein, or method of any of the preceding embodiments, wherein the serine recombinase polypeptide domain is provided as an mRNA encoding the serine recombinase polypeptide domain.

183. The system, fusion protein, or method of any of the preceding embodiments, wherein the template RNA is provided in an exosome.

184. The system, fusion protein, or method of any of the preceding embodiments, wherein the template RNA is provided in a proteinaceous exterior (e.g., comprised in a retroviral (e.g., lentiviral) particle, e.g., an integration-deficient retrovirus (e.g., lentivirus)), wherein the proteinaceous exterior is comprised in an exosome.

185. The system, fusion protein, or method of embodiment 184, wherein the serine recombinase polypeptide domain is provided in a polypeptide in the exosome.

186. The system, fusion protein, or method of embodiment 179 or 180, wherein the template RNA and the serine recombinase polypeptide domain are enclosed in different proteinaceous exteriors (e.g., comprised in different retroviral (e.g., lentiviral) particles, e.g., different integration-deficient retroviruses (e.g., lentiviruses)).

187. The system, fusion protein, or method of any of the preceding embodiments, wherein the system comprises:
(1) a first retroviral (e.g., lentiviral) particle (e.g., a first integration-deficient retrovirus (e.g., lentivirus)) comprising the template RNA; and
(2) a second retroviral (e.g., lentiviral) particle (e.g., a second integration-deficient retrovirus (e.g., lentivirus)) comprising the serine recombinase polypeptide domain.

188. The system, fusion protein, or method of embodiment 187, wherein the second retroviral (e.g., lentiviral) particle further comprises the retroviral (e.g., lentiviral) structural polypeptide domain and/or the retroviral (e.g., lentiviral) reverse transcriptase domain.

189. The system, fusion protein, or method of any of the preceding embodiments, wherein the system comprises a retroviral (e.g., lentiviral) particle (e.g., an integration-deficient retrovirus (e.g., lentivirus)) comprising the template RNA and the serine recombinase polypeptide domain; optionally wherein the retroviral (e.g., lentiviral) particle further comprises the retroviral (e.g., lentiviral) structural polypeptide domain and/or the retroviral (e.g., lentiviral) reverse transcriptase domain.

190. A lentiviral particle comprising a template RNA and serine recombinase (e.g., serine integrase) polypeptide domain;
wherein the template RNA comprises a DNA recognition sequence and a heterologous object sequence; wherein the integrase of the lentiviral particle is inactivated;
wherein the serine recombinase polypeptide domain comprises an amino acid sequence of any of SEQ ID NOs: 1-12,677 (e.g., any of SEQ ID NOs: 1-11,432), or an amino acid sequence having at least 70%, 75%, 80%, 85%, 90%, 95%, 96%, 97%, 98%, or 99% identity thereto,
and wherein the serine recombinase polypeptide domain binds the DNA recognition sequence and is capable of integrating the template DNA into the target DNA.

191. The system, fusion protein, or method of any of the preceding embodiments, wherein the retroviral (e.g., lentiviral) envelope polypeptide domain comprises a retroviral (e.g., lentiviral) env protein, gp120 protein, or gp41 protein.

192. The system, fusion protein, or method of embodiment 191, wherein the retroviral envelope polypeptide domain is a fusogen (e.g., a fusogen as described in any of PCT Publication Nos. WO2020014209, WO2020102485, and WO2020102503, which are herein incorporated by reference in their entirety).

193. The system, fusion protein, or method of of embodiment 191 or 192, wherein the retroviral envelope polypeptide domain promotes fusion between a viral envelope (e.g., comprising the retroviral polypeptide domain) and a membrane (e.g., a cell membrane).

194. The system, fusion protein, or method of any of the preceding embodiments, wherein the cognate DNA recognition sequence is identical in sequence to the DNA recognition sequence of the template nucleic acid, or differs by no more than 1, 2, 3, 4, 5, 6, 7, 8, 9, or 10 sequence alterations, or has at least 70%, 75%, 80%, 85%, 90%, 95%, 96%, 97%, 98%, or 99% identity thereto.

195. The system, fusion protein, or method of any of the preceding embodiments, wherein an RNA of the system (e.g., template RNA, the RNA encoding the polypeptide of (a), or an RNA expressed from a heterologous object sequence integrated into a target DNA) comprises a microRNA binding site, e.g., in a 3′ UTR.

196. The system, fusion protein, or method of embodiment 195, wherein the microRNA binding site is recognized by a miRNA that is present in a non-target cell type, but that is not present (or is present at a reduced level relative to the non-target cell) in a target cell type.

197. The system, fusion protein, or method of embodiment 195 or 196, wherein the miRNA is miR-142, and/or wherein the non-target cell is a Kupffer cell or a blood cell, e.g., an immune cell.

198. The system, fusion protein, or method of embodiment 195 or 196, wherein the miRNA is miR-182 or miR-183, and/or wherein the non-target cell is a dorsal root ganglion neuron.

199. The system, fusion protein, or method of any of embodiments 195-198, wherein the system comprises a first miRNA binding site that is recognized by a first miRNA (e.g., miR-142) and the system further comprises a second miRNA binding site that is recognized by a second miRNA (e.g., miR-182 or miR-183), wherein the first miRNA binding site and the second miRNA binding site are situated on the same RNA or on different RNAs of the system.

200. The system, fusion protein, or method of any of embodiments 195-199, wherein the template RNA comprises at least 2, 3, or 4 miRNA binding sites, e.g., wherein the miRNA binding sites are recognized by the same or different miRNAs.

201. The system, fusion protein, or method of any of embodiments 195-200, wherein the RNA encoding the polypeptide of (a) comprises at least 2, 3, or 4 miRNA binding sites, e.g., wherein the miRNA binding sites are recognized by the same or different miRNAs.

202. The system, fusion protein, or method of any of embodiments 195-201, wherein the RNA expressed from a heterologous object sequence integrated into a target DNA comprises at least 2, 3, or 4 miRNA binding sites, e.g., wherein the miRNA binding sites are recognized by the same or different miRNAs.

203. A method of modifying the genome of a cell (e.g., a eukaryotic cell, e.g., a mammalian cell, e.g., human cell), comprising contacting the cell with a composition comprising:
(i) the retroviral (e.g., lentiviral) structural polypeptide domain of a system of any of the preceding embodiments,
(ii) the retroviral (e.g., lentiviral) reverse transcriptase polypeptide domain of the system of any of the preceding embodiments,
(iii) the serine recombinase (e.g., serine integrase) polypeptide domain of the system of any of the preceding embodiments, and
(iv) the template RNA of the system of any of the preceding embodiments; thereby modifying the genome of the cell.

204. A method of modifying the genome of a cell (e.g., a eukaryotic cell, e.g., a mammalian cell, e.g., human cell) comprising contacting the cell with a composition comprising:
(i) the retroviral (e.g., lentiviral) structural polypeptide domain of a system of any of the preceding embodiments,
(ii) the retroviral (e.g., lentiviral) reverse transcriptase polypeptide domain of the system of any of the preceding embodiments, and
(iii) a nucleic acid molecule (e.g., an RNA molecule or a DNA molecule) encoding the serine recombinase (e.g., serine integrase) polypeptide domain of the system of any of the preceding embodiments, and
(iv) the template RNA of the system of any of the preceding embodiments; thereby modifying the genome of the cell.

205. The method of embodiment 204, wherein the nucleic acid molecule encoding the serine recombinase polypeptide domain is comprised in the template RNA.

206. The method of embodiment 204, wherein the nucleic acid molecule encoding the serine recombinase polypeptide domain is not comprised in the template RNA, e.g., is provided as a separate RNA.

207. The method of any of the preceding embodiments, wherein the cell comprises, in its genome, a cognate DNA recognition sequence (e.g., an endogenous DNA recognition sequence).

208. The method of embodiment 207, wherein the method results in insertion of the template DNA, or a portion thereof (e.g., into the cognate DNA recognition sequence.

209. The method of embodiment 207 or 208, wherein the cognate DNA recognition sequence is in a safe harbor site or a Natural Harbor™ site (e.g., as described in WO2020/047124, which is herein incorporated by reference in its entirety, including all description of Natural Harbor™ sites, including Table 4 therein; or as described in Aznauryan et al. (2022, Cell Reports Methods 2:10015), incorporated herein by reference in its entirety, including all description of genomic safe harbor sites, e.g., as shown in FIG. 1).

210. The method of embodiment 207 or 208, wherein the cognate DNA recognition sequence is in a gene associated with a disease, or is within 1 kb, 2 kb, 3 kb, 4 kb, 5 kb, or 10 kb of a gene associated with a disease.

211. The method of any of embodiments 207-210, wherein the cognate DNA recognition sequence comprises a nucleic acid sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432).

212. The system, fusion protein, or method of any of the preceding embodiments, wherein the system, polypeptide, and/or nucleic acid (e.g., RNA or DNA) encoding the same, is formulated as a lipid nanoparticle (LNP).

213. The system, fusion protein, or method of embodiment 212, wherein the lipid nanoparticle (or a formulation comprising a plurality of the lipid nanoparticles) lacks reactive impurities (e.g., aldehydes), or comprises less than a preselected level of reactive impurities (e.g., aldehydes).

214. The system, fusion protein, or method of embodiment 212, wherein the lipid nanoparticle (or a formulation comprising a plurality of the lipid nanoparticles) lacks aldehydes, or comprises less than a preselected level of aldehydes.

215. The system, fusion protein, or method of embodiment 212 or 213, wherein the lipid nanoparticle is comprised in a formulation comprising a plurality of the lipid nanoparticles.

216. The system, fusion protein, or method of embodiment 215, wherein the lipid nanoparticle formulation is produced using one or more lipid reagents comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.

217. The system, fusion protein, or method of embodiment 216, wherein the lipid nanoparticle formulation is produced using one or more lipid reagents comprising less than 3% total reactive impurity (e.g., aldehyde) content.

218. The system, fusion protein, or method of any of embodiments 215-217, wherein the lipid nanoparticle formulation is produced using one or more lipid reagents comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

219. The system, fusion protein, or method of embodiment 218, wherein the lipid nanoparticle formulation is produced using one or more lipid reagent comprising less than 0.3% of any single reactive impurity (e.g., aldehyde) species.

220. The system, fusion protein, or method of embodiment 219, wherein the lipid nanoparticle formulation is produced using one or more lipid reagents comprising less than 0.1% of any single reactive impurity (e.g., aldehyde) species.

221. The system, fusion protein, or method of any of embodiments 215-220, wherein the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.

222. The system, fusion protein, or method of embodiment 221, wherein the lipid nanoparticle formulation comprises less than 3% total reactive impurity (e.g., aldehyde) content.

223. The system, fusion protein, or method of any of embodiments 215-222, wherein the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

224. The system, fusion protein, or method of embodiment 223, wherein the lipid nanoparticle formulation comprises less than 0.3% of any single reactive impurity (e.g., aldehyde) species.

225. The system, fusion protein, or method of embodiment 223, wherein the lipid nanoparticle formulation comprises less than 0.1% of any single reactive impurity (e.g., aldehyde) species.

226. The system, fusion protein, or method of any of embodiments 212-225, wherein one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content.

227. The system, fusion protein, or method of embodiment 226, wherein one or more, or optionally all, of the lipid reagents used for a lipid nanoparticle as described herein or a formulation thereof comprise less than 3% total reactive impurity (e.g., aldehyde) content.

228. The system, fusion protein, or method of any of embodiments 212-227, wherein one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

229. The system, fusion protein, or method of embodiment 228, wherein one or more, or optionally all, of the lipid reagents used for a lipid nanoparticle as described herein or a formulation thereof comprise less than 0.3% of any single reactive impurity (e.g., aldehyde) species.

230. The system, fusion protein, or method of embodiment 228, wherein one or more, or optionally all, of the lipid reagents used for a lipid nanoparticle as described herein or a formulation thereof comprise less than 0.1% of any single reactive impurity (e.g., aldehyde) species.

231. The system, fusion protein, or method of any of embodiments 212-230, wherein the total aldehyde content and/or quantity of any single reactive impurity (e.g., aldehyde) species is determined by liquid chromatography (LC), e.g., coupled with tandem mass spectrometry (MS/MS), e.g., according to the method described in Example 26.

232. The system, fusion protein, or method of any of embodiments 212-230, wherein the total aldehyde content and/or quantity of reactive impurity (e.g., aldehyde) species is determined by detecting one or more chemical modifications of a nucleic acid molecule (e.g., as described herein) associated with the presence of reactive impurities (e.g., aldehydes), e.g., in the lipid reagents.

233. The system, fusion protein, or method of any of embodiments 212-230, wherein the total aldehyde content and/or quantity of aldehyde species is determined by detecting one or more chemical modifications of a nucleotide or nucleoside (e.g., a ribonucleotide or ribonucleoside, e.g., comprised in or isolated from a nucleic acid molecule, e.g., as described herein) associated with the presence of reactive impurities (e.g., aldehydes), e.g., in the lipid reagents, e.g., as described in Example 27.

234. The system, fusion protein, or method of embodiment 233, wherein the chemical modifications of a nucleic acid molecule, nucleotide, or nucleoside are detected by determining the presence of one or more modified nucleotides or nucleosides, e.g., using LC-MS/MS analysis, e.g., as described in Example 27.

235. A lipid nanoparticle (LNP) comprising the system, polypeptide (or RNA encoding the same), nucleic acid molecule, or DNA encoding the system or polypeptide, of any preceding embodiment.

236. A system comprising a first lipid nanoparticle comprising the polypeptide (or DNA or RNA encoding the same) of a Gene Writing system (e.g., as described herein); and a second lipid nanoparticle comprising a nucleic acid molecule of a Gene Writing System (e.g., as described herein).

237. The system, fusion protein, or method of any preceding embodiment, wherein the system, nucleic acid molecule, polypeptide, and/or DNA encoding the same, is formulated as a lipid nanoparticle (LNP).

238. The LNP of embodiment 237, comprising a cationic lipid.

239. The LNP of embodiment 237 or 238, wherein the cationic lipid has a structure according to:

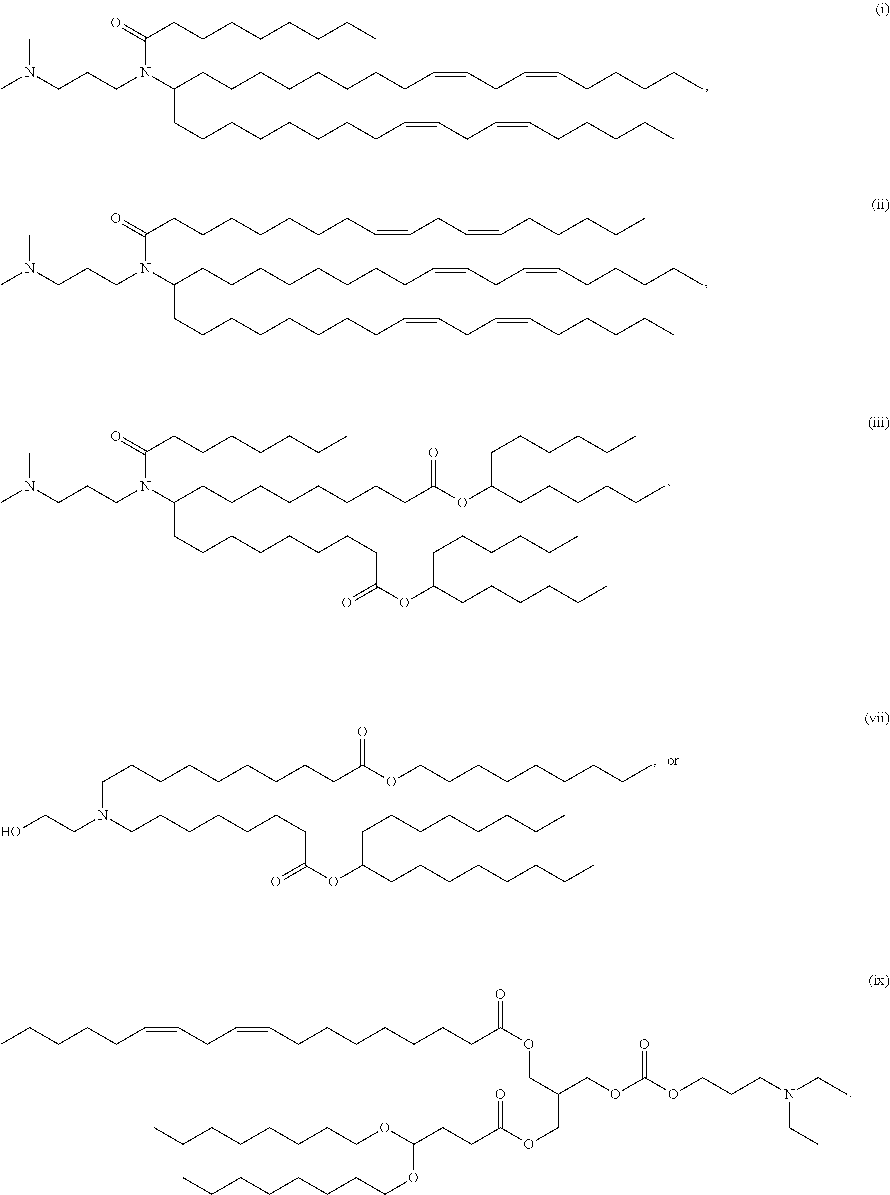

240. The LNP of any of embodiments 237-239, further comprising one or more neutral lipid, e.g., DSPC, DPPC, DMPC, DOPC, POPC, DOPE, SM, a steroid, e.g., cholesterol, and/or one or more polymer conjugated lipid, e.g., a pegylated lipid, e.g., PEG-DAG, PEG-PE, PEG-S-DAG, PEG-cer or a PEG dialkyoxypropylcarbamate.

241. The system, fusion protein, or method of any of the preceding embodiments, wherein the system comprises one or more circular RNA molecules (circRNAs).

242. The system, fusion protein, or method of embodiment 241, wherein the circRNA encodes the recombinase polypeptide, structural polypeptide domain, and/or reverse transcriptase polypeptide domain.

243. The system, fusion protein, or method of embodiment 241 or 242, wherein circRNA is delivered to a host cell.

244. The system, fusion protein, or method of any of the preceding embodiments, wherein the circRNA is capable of being linearized, e.g., in a host cell, e.g., in the nucleus of the host cell.

245. The system, fusion protein, or method of any of the preceding embodiments, wherein the circRNA comprises a cleavage site.

246. The system, fusion protein, or method of any embodiment 245, wherein the circRNA further comprises a second cleavage site.

247. The system, fusion protein, or method of embodiment 245 or 246, wherein the cleavage site can be cleaved by a ribozyme, e.g., a ribozyme comprised in the circRNA (e.g., by autocleavage).

248. The system, fusion protein, or method of any of the preceding embodiments, wherein the circRNA comprises a ribozyme sequence.

249. The system, fusion protein, or method of embodiment 248, wherein the ribozyme sequence is capable of autocleavage, e.g., in a host cell, e.g., in the nucleus of the host cell.

250. The system, fusion protein, or method of embodiment 248 or 249, wherein the ribozyme is an inducible ribozyme.

251. The system, fusion protein, or method of any of embodiments 248-250, wherein the ribozyme is a protein-responsive ribozyme, e.g., a ribozyme responsive to a nuclear protein, e.g., a genome-interacting protein, e.g., an epigenetic modifier, e.g., EZH2. 252. The system, fusion protein, or method of any of embodiments 248-251, wherein the ribozyme is a nucleic acid-responsive ribozyme.

253. The system, fusion protein, or method of embodiment 252, wherein the catalytic activity (e.g., autocatalytic activity) of the ribozyme is activated in the presence of a target nucleic acid molecule (e.g., an RNA molecule, e.g., an mRNA, miRNA, ncRNA, lncRNA, tRNA, snRNA, or mtRNA).

254. The system, fusion protein, or method of any of embodiments 248-251, wherein the ribozyme is responsive to a target protein (e.g., an MS2 coat protein).

255. The system, fusion protein, or method of embodiment 253, wherein the target protein localized to the cytoplasm or localized to the nucleus (e.g., an epigenetic modifier or a transcription factor).

256. The system, fusion protein, or method of any of embodiments 248-252, wherein the ribozyme comprises the ribozyme sequence of a B2 or ALU retrotransposon, or a nucleic acid sequence having at least 85%, 90%, 95%, 96%, 97%, 98%, or 99% sequence identity thereto.

257. The system, fusion protein, or method of any of embodiments 248-252, wherein the ribozyme comprises the sequence of a tobacco ringspot virus hammerhead ribozyme, or a nucleic acid sequence having at least 85%, 90%, 95%, 96%, 97%, 98%, or 99% sequence identity thereto.

258. The system, fusion protein, or method of any of embodiments 248-252, wherein the ribozyme comprises the sequence of a hepatitis delta virus (HDV) ribozyme, or a nucleic acid sequence having at least 85%, 90%, 95%, 96%, 97%, 98%, or 99% sequence identity thereto.

259. The system, fusion protein, or method of any of embodiments 248-258, wherein the ribozyme is activated by a moiety expressed in a target cell or target tissue.

260. The system, fusion protein, or method of any of embodiments 248-259, wherein the ribozyme is activated by a moiety expressed in a target subcellular compartment (e.g., a nucleus, nucleolus, cytoplasm, or mitochondria).

261. The system, fusion protein, or method of any of the preceding embodiments, wherein the ribozyme is comprised in a circular RNA or a linear RNA.

262. A system comprising a first circular RNA encoding the polypeptide of a Gene Writing system; and a second circular RNA comprising the template RNA of a Gene Writing system.

263. The system of any of the preceding embodiments, wherein the template RNA, e.g., the 5′ UTR, comprises a ribozyme which cleaves the template RNA (e.g., in the 5′ UTR).

264. The system of any of the preceding embodiments, wherein the template RNA comprises a ribozyme that is heterologous to (a)(i), (a)(ii), (b)(i), or a combination thereof.

265. The system of any of the preceding embodiments, wherein the heterologous ribozyme is capable of cleaving RNA comprising the ribozyme, e.g., 5′ of the ribozyme, 3′ of the ribozyme, or within the ribozyme.

266. The system, kit, polypeptide, cell, method, or reaction mixture of any of the preceding embodiments, wherein the insert DNA comprises:
(i) a first insulator;
(ii) the DNA recognition sequence; and
(iii) the heterologous object sequence.

267. A template nucleic acid molecule comprising:
(i) a first insulator;
(ii) a DNA recognition sequence that is specifically bound by a recombinase polypeptide (e.g., a tyrosine recombinase polypeptide or a serine recombinase polypeptide); and
(iii) a heterologous object sequence.

268. The template nucleic acid, system, kit, polypeptide, cell, method, or reaction mixture of embodiment 266 or 267, wherein (ii) is positioned between (i) and (iii).

269. The template nucleic acid, system, kit, polypeptide, cell, method, or reaction mixture of embodiment 266 or 267, wherein (i) is positioned between (ii) and (iii).

270. The template nucleic acid, system, kit, polypeptide, cell, method, or reaction mixture of embodiment 266-269, which further comprises (iv) a second insulator.

271. The template nucleic acid, system, kit, polypeptide, cell, method, or reaction mixture of embodiment 270, wherein (i)-(iv) are positioned in the following order: (i), (ii), (iv), (iii).

272. The template nucleic acid, system, kit, polypeptide, cell, method, or reaction mixture of any of the preceding embodiments, wherein the distance between the first insulator and the DNA recognition sequence is less than 2500, 2000, 1500, 1000, 750, 500, 400, 300, 200, 150, 100, 90, 80, 70, 60, 50, 40, 30, 20, 15, 10, 9, 8, 7, 6, 5, 4, 3, 2, or 1 nucleotides (e.g., is 0 nucleotides).

273. The template nucleic acid, system, kit, polypeptide, cell, method, or reaction mixture of the preceding embodiments, wherein the distance between the DNA recognition sequence and the second insulator is less than 2500, 2000, 1500, 1000, 750, 500, 400, 300, 200, 150, 100, 90, 80, 70, 60, 50, 40, 30, 20, 15, 10, 9, 8, 7, 6, 5, 4, 3, 2, or 1 nucleotides (e.g., is 0 nucleotides).

274. The template nucleic acid, system, kit, polypeptide, cell, method, or reaction mixture of any of the preceding embodiments, wherein the distance between the first insulator and the second insulator is less than 1000, 900, 800, 700, 600, 500, 400, 300, 200, 150, 100, 90, 80, 70, 60, or 50 nucleotides.

275. The template nucleic acid, system, kit, polypeptide, cell, method, or reaction mixture of any of the preceding embodiments, wherein when the template nucleic acid molecule or insert DNA is integrated into a target DNA molecule (e.g., genomic DNA, e.g., a chromosome or mitochondrial DNA), the nucleic acid sequence between the first insulator and the second insulator is insulated from one or more of:
a) heterochromatin formation;
b) epigenetic regulation (e.g., from both of epigenetic regulation and transcriptional regulation);
c) transcriptional regulation;
d) histone deacetylation (e.g., from both of histone deacetylation and histone methylation);
e) histone methylation;
f) histone deacetylation; and
g) DNA methylation, e.g., promoter DNA methylation.

276. The template nucleic acid, system, kit, polypeptide, cell, method, or reaction mixture of any of the preceding embodiments, wherein when the template nucleic acid molecule or insert DNA is integrated into a target DNA molecule (e.g., genomic DNA, e.g., a chromosome or mitochondrial DNA), the rate of heterochromatin formation of the nucleic acid sequence between the first insulator and the second insulator is reduced by at least about 5%, 10%, 15%, 20%, 25%, 30%, 40%, 50%, 60%, 70%, 75%, 80%, 85%, 90%, 95%, 96%, 97%, 98%, 99%, or 100% compared to an otherwise similar template nucleic acid or insert DNA that lacks the first and second insulators.

277. The template nucleic acid, system, kit, polypeptide, cell, method, or reaction mixture of any of the preceding embodiments, wherein when the template nucleic acid molecule or insert DNA is integrated into a target DNA molecule (e.g., genomic DNA, e.g., a chromosome or mitochondrial DNA), there is a difference (e.g., an increase or reduction) by at least about 5%, 10%, 15%, 20%, 25%, 30%, 40%, 50%, 60%, 70%, 75%, 80%, 85%, 90%, 95%, 96%, 97%, 98%, 99%, or 100% in the nucleic acid sequence on one side of the first insulator compared to the nucleic acid sequence on the other side of the first insulator, of one or more of:
a) heterochromatin formation;
b) epigenetic regulation (e.g., from both of epigenetic regulation and transcriptional regulation);
c) transcriptional regulation;
d) histone deacetylation (e.g., from both of histone deacetylation and histone methylation);
e) histone methylation;
f) histone deacetylation; and
g) DNA methylation, e.g., promoter DNA methylation.

278. The template nucleic acid, system, kit, polypeptide, cell, method, or reaction mixture of any of the preceding embodiments, wherein when the template nucleic acid molecule or insert DNA is integrated into a target DNA molecule (e.g., genomic DNA, e.g., a chromosome or mitochondrial DNA), there is a difference (e.g., an increase or reduction) by at least about 5%, 10%, 15%, 20%, 25%, 30%, 40%, 50%, 60%, 70%, 75%, 80%, 85%, 90%, 95%, 96%, 97%, 98%, 99%, or 100% in the nucleic acid sequence between the first and second insulators, compared to an otherwise similar nucleic acid sequence that is situated in the same site in the target DNA molecule and lacks the first and second insulator, of one or more of:
a) heterochromatin formation;
b) epigenetic regulation (e.g., from both of epigenetic regulation and transcriptional regulation);
c) transcriptional regulation;
d) histone deacetylation (e.g., from both of histone deacetylation and histone methylation);
e) histone methylation;
f) histone deacetylation; and
g) DNA methylation, e.g., promoter DNA methylation.

279. The template nucleic acid, system, kit, polypeptide, cell, method, or reaction mixture of any of the preceding embodiments, wherein when the template nucleic acid molecule or insert DNA is integrated into a target DNA molecule (e.g., genomic DNA, e.g., a chromosome or mitochondrial DNA), the level of heterochromatin formation in a predetermined time frame of the nucleic acid sequence between the first insulator and the second insulator is reduced by at least about 5%, 10%, 15%, 20%, 25%, 30%, 40%, 50%, 60%, 70%, 75%, 80%, 85%, 90%, 95%, 96%, 97%, 98%, 99%, or 100% compared to an otherwise similar template nucleic acid that lacks the first and second insulators, wherein optionally the predetermined time frame is 7, 10, 14, 21, 28, or 60 days.

280. The template nucleic acid, system, kit, polypeptide, cell, method, or reaction mixture of any of the preceding embodiments, wherein when the template nucleic acid molecule or insert DNA is integrated into a target DNA molecule (e.g., genomic DNA, e.g., a chromosome or mitochondrial DNA), the level of expression of a gene comprised in the heterologous object sequence is reduced by no more than 0.1%, 1%, 2%, 3%, 4%, 5%, 6%, 7%, 8%, 9%, 10%, 20%, 30%, 40%, 50%, or 75% compared to an otherwise similar template nucleic acid that lacks the first and second insulators.

281. The template nucleic acid, system, kit, polypeptide, cell, method, or reaction mixture of any of the preceding embodiments, wherein the first and/or second insulator is specifically bound by CTCF (CCCTC-binding factor), CTF (CAAT-binding transcription factor 1), USF1 (Upstream Stimulatory Factor 1), USF2 (Upstream Stimulatory Factor 2), PARP-1 (Poly(ADP-ribose) Polymerase-1), or VEZF1 (Vascular Endothelial Zinc Finger 1).

282. The template nucleic acid, system, kit, polypeptide, cell, method, or reaction mixture of any of the preceding embodiments, wherein the first and/or second insulator comprises the nucleic acid sequence of an insulator selected from any one of chicken P-globin 5′HS4 (cHS4) element, a Scaffold or Matrix Attachment Region (S/MAR) (e.g., MAR X_S29), a Stabilising Anti Repressor (STAR) element (e.g., STAR40), a D4Z4 insulator, A Ubiquitous Chromatin Opening Element (UCOE element) (e.g., aHNRPA2B1-CBX3 locus (A2UCOE), 3′UCOE, or SRF-UCOE), or a nucleic acid sequence having at least 75%, 80%, 85%, 90%, 95%, 96%, 97%, 98%, or 99% identity thereto.

283. The template nucleic acid, system, kit, polypeptide, cell, method, or reaction mixture of any of the preceding embodiments, wherein one or both of the first and second insulator is a barrier insulator.

284. The template nucleic acid, system, kit, polypeptide, cell, method, or reaction mixture of any of the preceding embodiments, wherein one or both of the first and second insulator is an enhancer-blocking insulator.

285. The template nucleic acid, system, kit, polypeptide, cell, method, or reaction mixture of any of the preceding embodiments, wherein one or both of the first and second insulator is a passive boundary element.

286. The template nucleic acid, system, kit, polypeptide, cell, method, or reaction mixture of any of the preceding embodiments, wherein one or both of the first and second insulator is an active chromatin remodeling element.

287. The template nucleic acid, system, kit, polypeptide, cell, method, or reaction mixture of any of the preceding embodiments, wherein the first and/or second insulator comprises an insulator sequence identified according to the method described in Liu et al. (2015, Nature Biotechnol. 33(2): 198-203; incorporated herein by reference).

288. The template nucleic acid, system, kit, polypeptide, cell, method, or reaction mixture of any of the preceding embodiments, wherein the first insulator and the second insulator share the same orientation.

289. The template nucleic acid, system, kit, polypeptide, cell, method, or reaction mixture of any of the preceding embodiments, wherein the first insulator and the second insulator have opposite orientations.

290. The template nucleic acid, system, kit, polypeptide, cell, method, or reaction mixture of any of the preceding embodiments, wherein the first insulator and the second insulator have the same nucleic acid sequence.

291. The template nucleic acid, system, kit, polypeptide, cell, method, or reaction mixture of any of the preceding embodiments, wherein the first insulator and the second insulator have different nucleic acid sequences.

292. The template nucleic acid, system, kit, polypeptide, cell, method, or reaction mixture of any of the preceding embodiments, wherein the template nucleic acid molecule is DNA.

293. The template nucleic acid, system, kit, polypeptide, cell, method, or reaction mixture of any of the preceding embodiments, wherein the template nucleic acid molecule is RNA.

294. The template nucleic acid, system, kit, polypeptide, cell, method, or reaction mixture of any of the preceding embodiments, wherein the template nucleic acid molecule or insert DNA is circular (e.g., circular and double stranded).

295. The template nucleic acid, system, kit, polypeptide, cell, method, or reaction mixture of any of the preceding embodiments, wherein the template nucleic acid molecule or insert DNA is linear.

296. The template nucleic acid, system, kit, polypeptide, cell, method, or reaction mixture of any of the preceding embodiments, wherein the template nucleic acid molecule or insert DNA comprises doggybone DNA (dbDNA) or closed-ended DNA (ceDNA).

296a. The template nucleic acid, system, kit, polypeptide, cell, method, or reaction mixture of any of the preceding embodiments, wherein the template nucleic acid molecule or insert DNA comprises a viral vector (e.g., an AAV vector, adenovirus vector, or retroviral vector).

297. The template nucleic acid, system, kit, polypeptide, cell, method, or reaction mixture of any of the preceding embodiments, wherein the template nucleic acid molecule or insert DNA comprises exactly one DNA recognition sequence.

298. The template nucleic acid, system, kit, polypeptide, cell, method, or reaction mixture of any of the preceding embodiments, wherein the template nucleic acid molecule or insert DNA comprises exactly two insulators.

299. The template nucleic acid, system, kit, polypeptide, cell, method, or reaction mixture of any of the preceding embodiments, wherein the template nucleic acid molecule or insert DNA further comprises a promoter.

300. The template nucleic acid, system, kit, polypeptide, cell, method, or reaction mixture of any of the preceding embodiments, wherein the template nucleic acid molecule or insert DNA comprises exactly one promoter.

301. The template nucleic acid, system, kit, polypeptide, cell, method, or reaction mixture of any of the preceding embodiments, wherein the template nucleic acid molecule or insert DNA comprises exactly one heterologous object sequence.

302. The template nucleic acid, system, kit, polypeptide, cell, method, or reaction mixture of any of the preceding embodiments, wherein the template nucleic acid molecule or insert DNA further comprises an enhancer.

303. The template nucleic acid, system, kit, polypeptide, cell, method, or reaction mixture of any of the preceding embodiments, wherein the template nucleic acid molecule or insert DNA further comprises a long terminal repeat (LTR), e.g., from a retrovirus or a lentivirus (e.g., HIV).

304. The template nucleic acid, system, kit, polypeptide, cell, method, or reaction mixture of any of the preceding embodiments, wherein the template nucleic acid molecule or insert DNA comprises one or both of a 5′ long terminal repeat (5′ LTR) and a 3′ long terminal repeat (3′ LTR).

305. The template nucleic acid, system, kit, polypeptide, cell, method, or reaction mixture of embodiment 304, wherein the 3′ UTR comprises a deletion of its U3 sequence.

306. The template nucleic acid, system, kit, polypeptide, cell, method, or reaction mixture of embodiment 304 or 305, wherein the first insulator is positioned in an LTR, e.g., in the 3′ LTR or the 5′ LTR.

307. The template nucleic acid, system, kit, polypeptide, cell, method, or reaction mixture of any of embodiments 304-306, wherein the first insulator is positioned in the 3′ UTR, e.g., at the position of the deletion of the U3 sequence.

308. The template nucleic acid, system, kit, polypeptide, cell, method, or reaction mixture of any of embodiments 304-306, wherein the first insulator is positioned in the 3′ UTR, and upon reverse transcription the first insulator sequence is present in both the 3′ UTR and 5′ UTR sequences of the resulting DNA.

309. The template nucleic acid, system, kit, polypeptide, cell, method, or reaction mixture of any of the preceding embodiments, wherein the template nucleic acid molecule or insert DNA further comprises an inverted terminal repeat (ITR), e.g., from an adeno-associated virus.

310. The template nucleic acid, system, kit, polypeptide, cell, method, or reaction mixture of any of embodiments 303-309, wherein the LTR or ITR is positioned between the heterologous object sequence and the first insulator.

311. The template nucleic acid, system, kit, polypeptide, cell, method, or reaction mixture of any of embodiments 303-310, wherein the LTR or ITR is positioned between the heterologous object sequence and the second insulator.

312. The template nucleic acid, system, kit, polypeptide, cell, method, or reaction mixture of any of embodiments 303-311, wherein the LTR or ITR is not positioned between the first insulator and the DNA recognition sequence.

313. The template nucleic acid, system, kit, polypeptide, cell, method, or reaction mixture of any of embodiments 303-312, wherein the LTR or ITR is not positioned between the second insulator and the DNA recognition sequence.

314. The template nucleic acid, system, kit, polypeptide, cell, method, or reaction mixture of any of embodiments 303-313, wherein the first insulator is positioned between the heterologous object sequence and the first LTR or ITR

315. The template nucleic acid, system, kit, polypeptide, cell, method, or reaction mixture of any of embodiments 303-314, wherein the second insulator is positioned between the heterologous object sequence and the second LTR or ITR

316. The template nucleic acid, system, kit, polypeptide, cell, method, or reaction mixture of any of embodiments 303-315, wherein the first and second insulators are positioned between the heterologous object sequence and the LTRs or ITRs.

317. The template nucleic acid, system, kit, polypeptide, cell, method, or reaction mixture of any of the preceding embodiments, wherein the DNA recognition sequence is specifically bound by a serine recombinase (e.g., serine integrase) polypeptide that comprises an amino acid sequence of any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto.

318. The template nucleic acid, system, kit, polypeptide, cell, method, or reaction mixture of any of the preceding embodiments, wherein the DNA recognition sequence comprises a nucleic acid sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

319. The template nucleic acid, system, kit, polypeptide, cell, method, or reaction mixture of any of the preceding embodiments, wherein the serine integrase polypeptide comprises an amino acid sequence of any of SEQ ID NOs: 1-12,677 (e.g., SEQ ID NOs: 1-11,432), or an amino acid sequence having at least 70%, 75%, 80%, 85%, 90%, 95%, 96%, 97%, 98%, or 99% identity thereto.

320. The template nucleic acid, system, kit, polypeptide, cell, method, or reaction mixture of any of the preceding embodiments, wherein the serine integrase polypeptide is a viral serine integrase polypeptide or a plasmid serine integrase.

321. The template nucleic acid, system, kit, polypeptide, cell, method, or reaction mixture of any of the preceding embodiments, wherein the DNA recognition sequence comprises a first parapalindromic sequence and a second parapalindromic sequence, and a core sequence situated between the first and second parapalindromic sequences.

322. The template nucleic acid, system, kit, polypeptide, cell, method, or reaction mixture of any of the preceding embodiments, wherein each parapalindromic sequence is about 15-35 or 20-30 nucleotides in length.

323. The template nucleic acid, system, kit, polypeptide, cell, method, or reaction mixture of any of the preceding embodiments, wherein the first and second parapalindromic sequences together comprise a parapalindromic region occurring within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleotide sequence having at least 70%, 75%, 80%, 85%, 90%, 95%, 96%, 97%, 98%, or 99% identity to said parapalindromic region, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

324. The template nucleic acid, system, kit, polypeptide, cell, method, or reaction mixture of any of the preceding embodiments, wherein the core sequence has a length of about 2-20 nucleotides.

325. The template nucleic acid, system, kit, polypeptide, cell, method, or reaction mixture of any of the preceding embodiments, wherein the heterologous object sequence comprises a sequence encoding an effector (e.g., a therapeutic effector).

326. The template nucleic acid, system, kit, polypeptide, cell, method, or reaction mixture of any of the preceding embodiments, wherein the effector is a polypeptide (e.g., a protein).

327. The template nucleic acid, system, kit, polypeptide, cell, method, or reaction mixture of any of the preceding embodiments, wherein the effector is a nucleic acid (e.g., a non-coding RNA, e.g., an siRNA or miRNA).

328. The template nucleic acid, system, kit, polypeptide, cell, method, or reaction mixture of any of the preceding embodiments, wherein the DNA recognition sequence is within 20, 25, 30, 40, 50, 60, 70, 80, 90, 100, 125, 150, 175, 200, 225, 250, 300, 350, 400, 450, or 500 nucleotides of the heterologous object sequence.

329. The template nucleic acid, system, kit, polypeptide, cell, method, or reaction mixture of any of the preceding embodiments, wherein the serine integrase polypeptide is capable of integrating the heterologous object sequence, the first insulator, and the second insulator into a target DNA molecule (e.g., a genomic DNA, e.g., a chromosome or mitochondrial DNA), e.g., at a specific target site.

330. The template nucleic acid, system, kit, polypeptide, cell, method, or reaction mixture of embodiment 15, wherein the heterologous object sequence is integrated into the target DNA molecule at an efficiency of at least about 0.1% (e.g., at least about 0.1%, 0.5%, 1%, 5%, 10%, 20%, 30%, 40%, 50%, 60%, 70%, 80%, 85%, 90%, 95%, 96%, 97%, 98%, 99%, or 100%) of a population of the cell, e.g., as measured in an assay of Example 5.

331. The template nucleic acid, system, kit, polypeptide, cell, method, or reaction mixture of any of the preceding embodiments, wherein the DNA recognition sequence is capable of being recombined by the serine integrase polypeptide with a cognate DNA recognition sequence in a naturally occurring human genome.

332. The template nucleic acid, system, kit, polypeptide, cell, method, or reaction mixture of embodiment 331, wherein the cognate DNA recognition sequence is in a safe harbor site or a Natural Harbor™ site (e.g., as described in WO2020/047124, which is herein incorporated by reference in its entirety, including all description of Natural Harbor™ sites, including Table 4 therein).

333. The template nucleic acid, system, kit, polypeptide, cell, method, or reaction mixture of embodiment 331 or 332, wherein the cognate DNA recognition sequence is in a gene associated with a disease, or is within 1 kb, 2 kb, 3 kb, 4 kb, 5 kb, or 10 kb of a gene associated with a disease.

334. The template nucleic acid, system, kit, polypeptide, cell, method, or reaction mixture of any of embodiments 331-333, wherein the cognate DNA recognition sequence comprises a nucleic acid sequence as listed of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleotide sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

335. A cell (e.g., a human cell) comprising (e.g., in a chromosome), in order:
a) a first recombinase transfer sequence;
b) a first insulator;
c) a heterologous object sequence;
d) a second insulator; and
e) a second recombinase transfer sequence.

336. The cell of embodiment 335, which further comprises a first LTR, e.g., between the heterologous object sequence and the second insulator.

337. The cell of embodiment 336, which further comprises a second LTR, e.g., between the first LTR and the second insulator.

The disclosure contemplates all combinations of any one or more of the foregoing aspects and/or embodiments, as well as combinations with any one or more of the embodiments set forth in the detailed description and examples.

### Definitions

About, approximately: “About” or “approximately” as the terms are used herein applied to one or more values of interest, refer to a value that is similar to a stated reference value. In certain embodiments, the term “approximately” or “about” refers to a range of values that fall within 15%, 14%, 13%, 12%, 11%, 10%, 9%, 8%, 7%, 6%, 5%, 4%, 3%, 2%, 1%, or less in either direction (greater than or less than) of the stated reference value unless otherwise stated or otherwise evident from the context (except where such number would exceed 100% of a possible value).

Domain: The term “domain” as used herein refers to a structure of a biomolecule that contributes to a specified function of the biomolecule. A domain may comprise a contiguous region (e.g., a contiguous sequence) or distinct, non-contiguous regions (e.g., non-contiguous sequences) of a biomolecule. Examples of protein domains include, but are not limited to, a nuclear localization sequence, a recombinase domain, a retroviral (e.g., lentiviral) structural polypeptide domain, a retroviral (e.g., lentiviral) lentiviral reverse transcriptase polypeptide domain, a DNA recognition domain (e.g., that binds to or is capable of binding to a recognition site, e.g. as described herein), a recombinase N-terminal domain (also called the catalytic domain), a C-terminal zinc ribbon domain, and domains listed in Table 1. In some embodiments the zinc ribbon domain further comprises a coiled-coiled motif. In some embodiments the recombinase domain and the zinc ribbon domain are collectively referred to as the C-terminal domain. In some embodiments the N-terminal domain is linked to the C-terminal domain by an αE linker or helix. In some embodiments the N-terminal domain is between 50 and 250 amino acids, or 100-200 amino acids, or 130-170 amino acids, e.g., about 150 amino acids. In some embodiments the C-terminal domain is 200-800 amino acids, or 300-500 amino acids. In some embodiments the recombinase domain is between 50 and 150 amino acids. In some embodiments the zinc ribbon domain is between 30 and 100 amino acids; an example of a domain of a nucleic acid is a regulatory domain, such as a transcription factor binding domain, a recognition sequence, an arm of a recognition sequence (e.g. a 5′ or 3′ arm), a core sequence, or an object sequence (e.g., a heterologous object sequence). In some embodiments, a recombinase polypeptide comprises one or more domains (e.g., a recombinase domain, or a DNA recognition domain) of a polypeptide comprising an amino acid sequence of any of SEQ ID NOs: 1-12,677 (e.g., SEQ ID NOs: 1-11,432), or a fragment or variant thereof. In some embodiments, a domain has a single enzymatic activity. In some embodiments, a domain has two or more enzymatic activities.

Exogenous: As used herein, the term exogenous, when used with reference to a biomolecule (such as a nucleic acid sequence or polypeptide) means that the biomolecule was introduced into a host genome, cell or organism by the hand of man. For example, a nucleic acid that is as added into an existing genome, cell, tissue or subject using recombinant DNA techniques or other methods is exogenous to the existing nucleic acid sequence, cell, tissue or subject.

Genomic safe harbor site (GSH site): A genomic safe harbor site is a site in a host genome that is able to accommodate the integration of new genetic material, e.g., such that the inserted genetic element does not cause significant alterations of the host genome posing a risk to the host cell or organism. A GSH site generally meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconserved element; (vi) has low transcriptional activity (i.e. no mRNA+/−25 kb); (vii) is not in a copy number variable region; (viii) is in open chromatin; and/or (ix) is unique, with 1 copy in the human genome. Examples of GSH sites in the human genome that meet some or all of these criteria include (i) the adeno-associated virus site 1 (AAVS1), a naturally occurring site of integration of AAV virus on chromosome 19; (ii) the chemokine (C—C motif) receptor 5 (CCR5) gene, a chemokine receptor gene known as an HIV-1 coreceptor; (iii) the human ortholog of the mouse Rosa26 locus; (iv) the rDNA locus. Additional GSH sites are known and described, e.g., in Pellenz et al. epub Aug. 20, 2018 (https://doi.org/10.1101/396390).

Heterologous: The term heterologous, when used to describe a first element in reference to a second element means that the first element and second element do not exist in nature disposed as described. For example, a heterologous polypeptide, nucleic acid molecule, construct or sequence refers to (a) a polypeptide, nucleic acid molecule or portion of a polypeptide or nucleic acid molecule sequence that is not native to a cell in which it is expressed, (b) a polypeptide or nucleic acid molecule or portion of a polypeptide or nucleic acid molecule that has been altered or mutated relative to its native state, or (c) a polypeptide or nucleic acid molecule with an altered expression as compared to the native expression levels under similar conditions. For example, a heterologous regulatory sequence (e.g., promoter, enhancer) may be used to regulate expression of a gene or a nucleic acid molecule in a way that is different than the gene or a nucleic acid molecule is normally expressed in nature. In certain embodiments, a heterologous nucleic acid molecule may exist in a native host cell genome, but may have an altered expression level or have a different sequence or both. In other embodiments, heterologous nucleic acid molecules may not be endogenous to a host cell or host genome but instead may have been introduced into a host cell by transformation (e.g., transfection, electroporation), wherein the added molecule may integrate into the host genome or can exist as extra-chromosomal genetic material either transiently (e.g., mRNA) or semi-stably for more than one generation (e.g., episomal viral vector, plasmid or other self-replicating vector).

Insulator: The term “insulator,” as used herein, refers to a cis-acting DNA sequence that functions as one or both of an enhancer-blocker or a heterochromatin barrier, or to a corresponding RNA sequence that, when reverse transcribed, produces the cis-acting DNA sequence. In some embodiments, an insulator is specifically bound by an insulator protein, which can bring the insulator into physical proximity with another insulator bound by an insulator protein (e.g., the same insulator protein). Generally, when a pair of insulators present on the same nucleic acid molecule are brought into proximity by insulator proteins, the insulators alter the activity and/or structure of the nucleic acid sequence between the two insulators. In some instances, the insulators reduce or block the formation of heterochromatin in the nucleic acid sequence between the insulators. In some instances, the insulators (e.g., by reducing or blocking heterochromatin formation) maintain or increase transcriptional activity of a heterologous object sequence positioned between the insulators. In some instances, the insulators reduce or block the pro-transcriptional activity of an enhancer positioned between the insulators. In some instances, the term “insulator” can refer to a DNA sequence that can function as an insulator (e.g., when paired with another insulator) or an RNA sequence that, when reverse transcribed, can form a DNA sequence that can function as an insulator. As used herein, the term “insulator protein” refers to a protein that specifically binds to an insulator sequence, e.g., a protein selected from CTCF (CCCTC-binding factor), CTF (CAAT-binding transcription factor 1), USF1 (Upstream Stimulatory Factor 1), USF2 (Upstream Stimulatory Factor 2), PARP-1 (Poly(ADP-ribose) Polymerase-1), and VEZF1 (Vascular Endothelial Zinc Finger 1), or a polypeptide having at least 75%, 80%, 85%, 90%, 95%, 96%, 97%, 98%, or 99% sequence identity thereto.

Integration-Deficient: The term “integration-deficient,” as used herein, refers to a viral system (e.g., a composition comprising a virus or viral vector) or a polypeptide thereof is substantially unable to integrate a template DNA into a target DNA (e.g., a genomic DNA, e.g., a chromosome or mitochondrial DNA). In some instances, an integration-deficient viral system comprises a mutation to a viral integrase (e.g., as described herein), a template RNA lacking a wild-type viral LTR sequence, or an inhibitor of the viral integrase. In some instances, an integration-deficient viral system results in a decrease in the level of integrated template DNA relative to an otherwise similar integration-competent viral system by at least 75%, 80%, 85%, 90%, 95%, 96%, 97%, 98%, 99%, 99.9%, or 100%.

Mutation or Mutated: The term “mutated” when applied to nucleic acid sequences means that nucleotides in a nucleic acid sequence may be inserted, deleted or changed compared to a reference (e.g., native) nucleic acid sequence. A single alteration may be made at a locus (a point mutation) or multiple nucleotides may be inserted, deleted or changed at a single locus. In addition, one or more alterations may be made at any number of loci within a nucleic acid sequence. A nucleic acid sequence may be mutated by any suitable method.

Nucleic acid molecule: Nucleic acid molecule refers to both RNA and DNA molecules including, without limitation, cDNA, genomic DNA and mRNA, and also includes synthetic nucleic acid molecules, such as those that are chemically synthesized or recombinantly produced, such as DNA templates, as described herein. The nucleic acid molecule can be double-stranded or single-stranded, circular or linear. If single-stranded, the nucleic acid molecule can be the sense strand or the antisense strand. Unless otherwise indicated, and as an example for all sequences described herein under the general format “SEQ ID NO:,” “nucleic acid comprising SEQ ID NO:1” refers to a nucleic acid, at least a portion which has either (i) the sequence of SEQ ID NO:1, or (ii) a sequence complimentary to SEQ ID NO:1. The choice between the two is dictated by the context in which SEQ ID NO:1 is used. For instance, if the nucleic acid is used as a probe, the choice between the two is dictated by the requirement that the probe be complimentary to the desired target. Nucleic acid sequences of the present disclosure may be modified chemically or biochemically or may contain non-natural or derivatized nucleotide bases, as will be readily appreciated by those of skill in the art. Such modifications include, for example, labels, methylation, substitution of one or more naturally occurring nucleotides with an analog, inter-nucleotide modifications such as uncharged linkages (for example, methyl phosphonates, phosphotriesters, phosphoramidates, carbamates, etc.), charged linkages (for example, phosphorothioates, phosphorodithioates, etc.), pendant moieties, (for example, polypeptides), intercalators (for example, acridine, psoralen, etc.), chelators, alkylators, and modified linkages (for example, alpha anomeric nucleic acids, etc.). Also included are synthetic molecules that mimic polynucleotides in their ability to bind to a designated sequence via hydrogen bonding and other chemical interactions. Such molecules are known in the art and include, for example, those in which peptide linkages substitute for phosphate linkages in the backbone of a molecule. Other modifications can include, for example, analogs in which the ribose ring contains a bridging moiety or other structure such as modifications found in “locked” nucleic acids.

Gene expression unit: a gene expression unit is a nucleic acid sequence comprising at least one regulatory nucleic acid sequence operably linked to at least one effector sequence. A first nucleic acid sequence is operably linked with a second nucleic acid sequence when the first nucleic acid sequence is placed in a functional relationship with the second nucleic acid sequence. For instance, a promoter or enhancer is operably linked to a coding sequence if the promoter or enhancer affects the transcription or expression of the coding sequence. Operably linked DNA sequences may be contiguous or non-contiguous. Where necessary to join two protein-coding regions, operably linked sequences may be in the same reading frame.

Host: The terms host genome or host cell, as used herein, refer to a cell and/or its genome into which protein and/or genetic material has been introduced. It should be understood that such terms are intended to refer not only to the particular subject cell and/or genome, but to the progeny of such a cell and/or the genome of the progeny of such a cell. Because certain modifications may occur in succeeding generations due to either mutation or environmental influences, such progeny may not, in fact, be identical to the parent cell, but are still included within the scope of the term “host cell” as used herein. A host genome or host cell may be an isolated cell or cell line grown in culture, or genomic material isolated from such a cell or cell line, or may be a host cell or host genome which composing living tissue or an organism. In some instances, a host cell may be an animal cell or a plant cell, e.g., as described herein. In certain instances, a host cell may be a bovine cell, horse cell, pig cell, goat cell, sheep cell, chicken cell, or turkey cell. In certain instances, a host cell may be a corn cell, soy cell, wheat cell, or rice cell.

Recombinase polypeptide: As used herein, a recombinase polypeptide refers to a polypeptide having the functional capacity to catalyze a recombination reaction of a nucleic acid molecule (e.g., a DNA molecule). A recombination reaction may include, for example, one or more nucleic acid strand breaks (e.g., a double-strand break), followed by joining of two nucleic acid strand ends (e.g., sticky ends). In some instances, the recombination reaction comprises insertion of an insert nucleic acid, e.g., into a target site, e.g., in a genome or a construct. In some instances, the recombination reaction comprises flipping or reversing of a nucleic acid, e.g., in a genome or a construct. In some instances, the recombination reaction comprises removing a nucleic acid, e.g., from a genome or a construct. In some instances, a recombinase polypeptide comprises one or more structural elements of a naturally occurring recombinase (e.g., a serine recombinase, e.g., PhiC31 recombinase or Gin recombinase). In certain instances, a recombinase polypeptide comprises an amino acid sequence having at least 70%, 75%, 80%, 85%, 90%, 95%, 96%, 97%, 98%, 99%, or 100% sequence identity to a recombinase described herein (e.g., an amino acid sequence of any of SEQ ID NOs: 1-12,677 (e.g., SEQ ID NOs: 1-11,432)). Typically, a serine recombinase uses a serine residue in nucleophilic attack of DNA, while a tyrosine recombinase uses a tyrosine residue in nucleophilic attack of DNA. In some embodiments, a recombinase polypeptide comprises a serine recombinase, e.g., a serine integrase. In some embodiments, a serine recombinase, e.g., a serine integrase, comprises one or more (e.g., all) of a recombinase domain, a catalytic domain, or a zinc ribbon domain. In some embodiments, a serine recombinase, e.g., a serine integrase, comprises a domain listed in Table 1 (e.g., either in addition to or in replacement of one or more of a recombinase domain, a catalytic domain, or a zinc ribbon domain). In some instances, a recombinase polypeptide has one or more functional features of a naturally occurring recombinase (e.g., a serine recombinase, e.g., PhiC31 recombinase or Gin recombinase). In some embodiments, a recombinase polypeptide is 350-900 amino acids, or 425-700 amino acids. In some instances, a recombinase polypeptide recognizes (e.g., binds to) a recognition sequence in a nucleic acid molecule (e.g., a recognition sequence occurring in a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a sequence having at least 70%, 75%, 80%, 85%, 90%, 95%, 96%, 97%, 98%, or 99% identity thereto). In some embodiments, the recombinase may facilitate recombination between a first recognition sequence (e.g. attB or pseudo-attB) and a second genomic recognition sequence (e,g., attP or pseudo attP). In some embodiments, one or more recognition sequences comprise an attP half site (e.g., attPL or attPR) sequence or an attB half site (e.g., attBL or attBR) sequence as listed in Table 26. In some embodiments, a recombinase polypeptide is not active as an isolated monomer. In some embodiments, a recombinase polypeptide catalyzes a recombination reaction in concert with one or more other recombinase polypeptides (e.g., two or four recombinase polypeptides per recombination reaction). In some embodiments, a recombinase polypeptide is active as a dimer. In some embodiments, a recombinase assembles as a dimer at the recognition sequence. In some embodiments, a recombinase polypeptide is active as a tetramer. In some embodiments, a recombinase assembles as a tetramer at the recognition sequence. In some embodiments, a recombinase polypeptide is a recombinant (e.g., a non-naturally occurring) recombinase polypeptide. In some embodiments, a recombinant recombinase polypeptide comprises amino acid sequences derived from a plurality of recombinase polypeptides (e.g., a recombinant recombinase polypeptide comprises a first domain from a first recombinase polypeptide and a second domain from a second recombinase polypeptide).

DNA recognition sequence: A “DNA recognition sequence” refers to a DNA sequence that is recognized (e.g., capable of being bound by) a recombinase polypeptide, e.g., as described herein, as well as to an RNA sequence that can be reverse transcribed to yield the DNA sequence that is recognized by the recombinase polypeptide. The DNA recognition sequences are, in some instances, generically referred to as attB and attP. DNA recognition sequences can be native or altered relative to a native sequence. In some instances, a recombinase polypeptide recognizes a DNA recognition sequence (e.g., in a template DNA, e.g., as described herein) and a cognate recognition sequence (e.g., a cognate DNA recognition sequence, e.g., in a target nucleic acid, e.g., a genomic DNA, e.g., a chromosome of mitochondrial DNA), and optionally induces recombination specifically between the DNA recognition sequence and the cognate recognition sequence. In some instances, the cognate recognition sequence occurs naturally in the genomic DNA (i.e., the cognate recognition sequence is present in the genomic DNA without previous manipulation by, e.g., genetic engineering techniques). The DNA recognition sequence may vary in length, but typically ranges from about 20 to about 200 nt, from about 30 to 90 nt, more usually from 30 to 70 nucleotides. DNA recognition sequences are typically arranged as follows: AttB comprises a first DNA sequence attB5′, a core region, and a second DNA sequence attB3′, in the relative order from 5′ to 3′ attB5′-core region-attB3′. AttP comprises a first DNA sequence attP5′, a core region, and a second DNA sequence attP3′, in the relative order from 5′ to 3′ attP5′-core region-attP3′. In some embodiments, the attB5′ and attB3′ are parapalindromic (e.g., one sequence is a palindrome relative to the other sequence or has at least 20%, 30%, 40%, 50%, 60%, 70%, 75%, 80%, 85%, 90%, 95%, 96%, 97%, 98%, or 99% sequence identity to a palindrome relative to the other sequence). In some embodiments, the attP5′ and attP3′ recognition sequences are parapalindromic (e.g., one sequence is a palindrome relative to the other sequence or has at least 20%, 30%, 40%, 50%, 60%, 70%, 75%, 80%, 85%, 90%, 95%, 96%, 97%, 98%, or 99% sequence identity to a palindrome relative to the other sequence). In some embodiments the attB5′ and attB3′ recognition sequences are parapalindromic to each other and the attP5′ and attP3′ recognition sequences are parapalindromic to each other. In some embodiments, the attB5′ and attB3′, and the attP5′ and attP3′ sequences are similar but not necessarily the same number of nucleotides. Because attB and attP are different sequences, recombination will result in a stretch of nucleic acids (called attL or attR for left and right) that is neither an attB sequence nor an attP sequence. Without wishing to be bound by theory, the dissimilarities between attL/attR and attB/attP probably make attL and attR sites less unrecognizable as a recombination site to the relevant recombinase enzyme, thus reducing the possibility that the enzyme will catalyze a second recombination reaction that would reverse the first. DNA recognition sequences are typically bound by a recombinase dimer. In some embodiments, one or more of the αE helix, the recombinase domain, the linker domain, and/or the zinc ribbon domain of the recombinase polypeptide contact the recognition sequence. In some instances, a recognition sequence comprises a nucleic acid sequence occurring within a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), e.g., a 20-200 nt sequence within a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), e.g., a 30-70 nt sequence within a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a sequence having at least 50%, 60%, 70%, 75%, 80%, 85%, 90%, 95%, 96%, 97%, 98%, or 99% identity thereto. In some instances, a recognition sequence comprises a nucleic acid sequence occurring within an attP (e.g., attPL or attPR) sequence listed in Table 26. In some instances, a recognition sequence comprises a nucleic acid sequence occurring within an attB (e.g., attBL or attBR) sequence listed in Table 26. In some embodiments, one or more recognition sequences comprise two attP half site (e.g., an attPL and an attPR) sequences or two attB half site (e.g., an attBL and an attBR) sequences as listed in Table 26.

Recombinase transfer sequence: “Recombinase transfer sequence” as used herein refers to a sequence constructed from portions of two DNA recognition sequences. In some embodiments, the sequence 5′ of the core sequence, e.g., the attB5′ or attP5′, of the recombinase transfer sequence matches a cognate recognition sequence (e.g., in the human genome) and the sequence 3′ of the core sequence, e.g., the attB3′ or attP3′, of the recombinase transfer sequence matches a DNA recognition sequence (e.g., in the template DNA). In some embodiments, the sequence 5′ of the core sequence, e.g., the attB5′ or attP5′, of the recombinase transfer sequence matches a DNA recognition sequence and the sequence 3′ of the core sequence, e.g., the attB3′ or attP3′, of the recombinase transfer sequence matches the cognate recognition sequence. In some embodiments, the sequence 5′ of the core sequence, e.g., the attB5′ or attP5′, of the recombinase transfer sequence matches a cognate recognition sequence and the sequence 3′ of the core sequence, e.g., the attB3′ or attP3′, of the recombinase transfer sequence matches a DNA recognition sequence. In some embodiments, the recombinase transfer sequence may be comprised of the region 5′ of the core sequence from a wild-type attB site and the region 3′ of the core sequence from a DNA attP recognition sequence, or vice versa. Other combinations of such recombinase transfer sequence will be evident to those having ordinary skill in the art, in view of the teachings of the present specification. In some embodiments, a recombinase described herein catalyzes recombination between a DNA recognition sequence and a cognate recognition sequence to yield a recombinase transfer sequence. In some embodiments, a recombinase described herein acts preferentially on a DNA recognition sequence relative to a recombinase transfer sequence. In some embodiments, a recombination directionality factor (RDF) is capable of modifying the preference of a recombinase described herein such that it preferentially acts on a recombinase transfer sequence relative to a DNA recognition sequence. In some embodiments, a DNA recognition sequence may be referred to as an attP or attB sequence, where a recombinase transfer sequences may be referred to as an attL or attR sequence.

Core sequence: A core sequence, as used herein, refers to a nucleic acid sequence positioned between two arms of a DNA recognition sequence, e.g., between a pair of parapalindromic sequences. In some embodiments, a core sequence is positioned between a attB5′ and an attB3′, or between an attP5′ and an attP3′. In some instances, a core sequence can be cleaved by a recombinase polypeptide (e.g., a recombinase polypeptide that recognizes a recognition sequence comprising the two parapalindromic sequences), e.g., to form sticky ends, e.g. a 3′ overhang. In some embodiments, the core sequence of the attB and attP are identical. In some embodiments, the core sequence of the attB and attP are not identical, e.g., have less than 99, 95, 90, 80, 70, 60, 50, 40, 30, or 20% identity. In some embodiments, the core sequence is about 2-20 nucleotides, e.g., 2-16 nucleotides, e.g., about 4 nucleotides in length or about 2 nucleotides in length (e.g., exactly 2 nucleotides in length). In some embodiments, a core sequence comprises a core dinucleotide corresponding to two adjacent nucleotides wherein a recombinase recognizing the nearby parapalindromic sequences may cut the DNA on one side of the core dinucleotide, e.g., forming sticky ends. In some embodiments, the core dinucleotide of the core sequence of an attB and/or attP site are identical, e.g., cleavage of the attP and/or attB sites form compatible sticky ends. In some embodiments, sequence identity between two DNA recognition sites, e.g., an attP and an attB site, is limited to the core sequence of the sites, e.g., is limited to a central dinucleotide. In some embodiments, a core sequence comprises a nucleic acid sequence occurring within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432). In some embodiments, a core sequence comprises a nucleic acid sequence not originating within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432). In some embodiments, one or more recognition sequences comprise two attP half site (e.g., an attPL and an attPR) sequences as listed in Table 26, further comprising a core sequence according to any of the embodiments herein. In some embodiments, one or more recognition sequences comprise two attB half site (e.g., an attBL and an attBR) sequences as listed in Table 26, further comprising a core sequence according to any of the embodiments herein.

Object sequence: As used herein, the term object sequence refers to a nucleic acid segment that can be desirably inserted into a target nucleic acid molecule, e.g., by a recombinase polypeptide, e.g., as described herein. In some embodiments, a template RNA or template DNA comprises a DNA recognition sequence and an object sequence that is heterologous to the DNA recognition sequence and/or the remainder of the template RNA or template DNA, generally referred to herein as a “heterologous object sequence.” An object sequence may, in some instances, be heterologous relative to the nucleic acid molecule into which it is inserted (e.g., a target DNA molecule, e.g., as described herein). In some instances, an object sequence comprises a nucleic acid sequence encoding a gene (e.g., a eukaryotic gene, e.g., a mammalian gene, e.g., a human gene) or other cargo of interest (e.g., a sequence encoding a functional RNA, e.g., an siRNA or miRNA), e.g., as described herein. In certain instances, the gene encodes a polypeptide (e.g., a blood factor or enzyme). In some instances, an object sequence comprises one or more of a nucleic acid sequence encoding a selectable marker (e.g., an auxotrophic marker or an antibiotic marker), and/or a nucleic acid control element (e.g., a promoter, enhancer, or silencer).

Parapalindromic: As used herein, the term “parapalindromic” refers to a property of a pair of nucleic acid sequences, wherein one of the nucleic acid sequences is either a palindrome relative to the other nucleic acid sequence, or has at least 20% (e.g., at least 20%, 30%, 35%, 40%, 45%, 50%, 55%, 60%, 65%, 70%, 75%, 80%, 85%, 90%, 95%, 96%, 97%, 98%, 99%, or 100%), e.g., at least 50%, sequence identity to a palindrome relative to the other nucleic acid sequence, or has no more than 1, 2, 3, 4, 5, 6, 7, 8, 9, 10, 11, 12, 13, 14, 15, 16, 17, 18, 19, or 20 sequence mismatches relative to the other nucleic acid sequence. “Parapalindromic sequences,” as used herein, refer to at least one of a pair of nucleic acid sequences that are parapalindromic relative to each other. A “parapalindromic region,” as used herein, refers to a nucleic acid sequence, or the portions thereof, that comprise two parapalindromic sequences. In some instances, a parapalindromic region comprises two parapalindromic sequences flanking a nucleic acid segment, e.g., comprising a core sequence.

Structural polypeptide domain: As used herein, the term “structural polypeptide domain” refers to a polypeptide domain that can form part of a proteinaceous exterior (e.g., a viral capsid) encapsulating a viral nucleic acid (e.g., a template RNA, e.g., as described herein). In some instances, a structural polypeptide domain is encoded by a viral gene (e.g., a retroviral gag gene). In some instances, a structural polypeptide domain comprises a capsid protein (e.g., a CA protein and/or an NC protein, e.g., encoded by a retroviral gag gene), or a functional fragment thereof. In some instances, a structural polypeptide domain comprises a matrix protein (e.g., a MA protein, e.g., encoded by a retroviral gag gene), or a functional fragment thereof. In some instances, a structural polypeptide domain comprises a domain encoded by a retroviral gag (e.g., a lentiviral gag). In some embodiments, a structural polypeptide domain comprises one or more mutations (e.g., point mutations, additions, substitutions, or deletions) relative to the amino acid sequence of a corresponding wild-type protein (e.g., a wild-type retroviral gag, CA, NC, or MA protein). In some embodiments, a structural polypeptide domain is part of a polyprotein or a fusion protein. In some embodiments, a structural polypeptide domain is not part of a polyprotein or a fusion protein.

Reverse transcriptase domain: As used herein, the term “reverse transcriptase domain” refers to a polypeptide domain capable of producing complementary DNA from a template RNA (e.g., as described herein). In some instances, a reverse transcriptase domain comprises a viral (e.g., retroviral, e.g., lentiviral) reverse transcriptase, or a functional fragment thereof. In some instances, a reverse transcriptase domain produces complementary DNA from a template RNA via a primer (e.g., a tRNA primer, e.g., a lysyl tRNA primer). In some instances, a reverse transcriptase domain produces a double stranded template DNA (e.g., as described herein) from the template RNA. In some instances, a reverse transcriptase domain is encoded by a viral (e.g., retroviral, e.g., lentiviral)pol gene. In some instances, a reverse transcriptase domain is encoded by a pol gene that also encodes a viral (e.g., retroviral, e.g., lentiviral) integrase (IN). In some instances, a reverse transcriptase domain is encoded by a pol gene that also encodes a viral (e.g., retroviral, e.g., lentiviral) protease (PR) and/or dTUPase (DU). In some embodiments, a reverse transcriptase polypeptide domain comprises one or more mutations (e.g., point mutations, additions, substitutions, or deletions) relative to the amino acid sequence of a corresponding wild-type protein (e.g., a wild-type retroviral pol, IN, PR, or DU protein). In some embodiments, the reverse transcriptase domain comprises RNaseH activity. In some embodiments, a functional reverse transcriptase comprises a single protein subunit, e.g., is monomeric. In some embodiments, a functional reverse transcriptase comprises at least two subunits, e.g., is dimeric. In some embodiments, the reverse transcriptase domain is less active (or inactive) in monomeric form compared to in dimeric form. In some embodiments, a dimeric reverse transcriptase comprises two identical subunits. In some embodiments, a dimeric reverse transcriptase comprises different subunits, e.g., a p51 and a p66 subunit. In some embodiments, a reverse transcriptase comprises at least three subunits, e.g., two p51 subunits and at least one p15 subunit. In some embodiments, a reverse transcriptase comprises an RNase H domain. In some embodiments, a reverse transcriptase comprises an inactivated RNase H domain. In some embodiments, a reverse transcriptase does not comprise an RNase H domain. In some embodiments, a reverse transcriptase domain is part of a polyprotein or a fusion protein. In some embodiments, a reverse transcriptase domain is not part of a polyprotein or a fusion protein.

### BRIEF DESCRIPTION OF THE DRAWINGS

FIG. 1A: Activity of 10 exemplary serine integrases in human cells. HEK293T cells were transfected with an integrase expression plasmid and a template plasmid harboring a 520 bp attP containing region followed by an EGFP reporter driven by CMV promoter. Shown are the percentage of EGFP-positive cells observed by flow cytometry at 21 days post-transfection.

FIG. 1B: Strategies to assess integration, stability, and expression of different AAV donor formats. A single attB* or attP* donor utilizes formation of double-stranded circularized DNA following AAV transduction into the cell nucleus. This configuration also includes ITR sequences post-integration. A dual attB-attB* or attP-attP* donor does not require formation of double-stranded circularized DNA following AAV transduction. The readout for integration stability and expression uses droplet digital PCR (ddPCR) and flow cytometry (FLOW).

FIG. 2: AAV constructs illustration. First line shows: ITR, stuffer (500), attP*, PEFla, EGFP, WPRE, hGHpA, ITR; AAV2 serotype. Second line shows: ITR, stuffer (500), attP, PEFla, EGFP, WPRE, hGHpA, attP*, stuffer (500), ITR; AAV2 serotype. Third line shows: ITR, stuffer (500), attB*, PEFla, EGFP, WPRE, hGHpA, ITR; AAV2 serotype. Fourth line shows: ITR, stuffer (500), attB, PEFla, EGFP, WPRE, hGHpA, attB*, stuffer (500), ITR; AAV2 serotype. Fifth line shows: ITR, PEFla, hcoBXB1, WPRE, hGHpA, ITR; AAV2 serotype. Sixth line shows: ITR, PEFla, mcoBXB1, WPRE, hGHpA, ITR; AAV6 serotype.

FIGS. 3A and 3B: Dual AAV delivery of serine integrase and template DNA to mammalian cells. (A) Schematic representation of experiment. BXB1 serine recombinase and template DNA are co-delivered as separate AAV viral vectors into BXB landing pad cell lines. (B) Droplet digital PCR (ddPCR) assay to assess integration (% CNV/landing pad) of BXB1 serine recombinase and transgene into attP-attP* landing pad cell line 3 days and 7 days post-transduction. Black dots (to the right of each pair of gray dots) indicate template only samples and fall at 0% on the y-axis. Gray dots (to the left of each pair of black dots) indicate template+BXB1 integrase and fall between 1-6% on the y-axis.

FIGS. 4A and 4B: mRNA delivery of BXB1 integrase and AAV delivery of template DNA to mammalian cells. (A) Schematic representation of experiment. mRNA delivery of BXB1 serine recombinase and AAV delivery of template DNA into BXB1 landing pad cell lines. (B) Droplet digital PCR (ddPCR) assay to assess integration (% CNV/landing pad) of BXB1 serine recombinase and transgene into attP-attP* landing pad cell line 3 days post mRNA transfection/AAV transduction. Black dots (to the right of each pair of gray dots) indicate template only samples and fall at 0% on the y-axis. Gray dots (to the left of each pair of black dots) indicate template+BXB1 integrase and fall at greater than 0% on the y-axis.

FIGS. 5A and 5B: General structure of recombinase recognition sites and presence of recognition sites in LeftRegion and RightRegion sequences disclosed herein. (A) General features of a recognition sequence. Serine recombinases as defined herein generally comprise a central dinucleotide, a core sequence, and flanking arms that may be parapalindromic in nature. Depicted here are the attP and attB recognition sequences for Bxb1 recombinase (e.g., a recombinase comprising an amino acid sequence of SEQ ID NO: 11,636 (though the general approach can also be applied to, e.g., SEQ ID NOs: 1-12,677, e.g., SEQ ID NOs: 1-11,432)). These sequences share the central dinucleotide, indicated in bold, which is important for successful recombination between the two sites. The arms of the recognition sites, indicated by black box outlines, may share palindromic sequences to a varying degree, thus being referred to as “parapalindromic” herein. Nucleotides that are palindromic with respect to the opposite arm are indicated by underlined text. Additionally, recognition sequences share a core that is common between the attP and attB site, indicated here by gray shading. The core sequence comprises the central dinucleotide at a minimum, but may include additional sequence. (B) The LeftRegion or RightRegion (e.g., comprising a sequence of any of SEQ ID NOs: 13,001-25,677 and SEQ ID NOs: 26,001-38,677, respectively, e.g., e.g., SEQ ID NOs: 24,636 and 37,636, respectively) comprises the attP site for a cognate recombinase. SEQ ID NOs: 13,001-25,677 (e.g., SEQ ID NOs: 13,001-24,432) or SEQ ID NOs: 26,001-38,677 (e.g., SEQ ID NOs: 26,001-37,432)comprise exemplary recognition sites for exemplary recombinases described herein. As an example, the attP site for a recombinase of SEQ ID NO: n, wherein n is chosen from 1-12,677 (e.g., from 1-11,432), is found in SEQ ID NO: (n+13,000) (e.g., a LeftRegion) or SEQ ID NO: (n+26,000) (e.g., a RightRegion). Shown here, the attP site for Bxb1 integrase (e.g., an integrase comprising a sequence of SEQ ID NO: 11,636) can be found in the corresponding SEQ ID NO: 24,636 (e.g., a LeftRegion) and SEQ ID NO: 37,636 (e.g., a RightRegion). The attP site of Bxb1 is shown as underlined and bolded text in the LeftRegion sequence.

FIG. 6: Schematic representations of the third generation IDLV-attP vectors. Exemplary IDLV vectors comprising a self-inactivating 3′LTR, a psi sequence (Ψ) allows for efficient incorporation of the vector RNA genome into particles, a Rev responsive element (RRE), a central polypurine tract (cPPT), the expression of EGFP transgene driven by human EF1a promoter, as well as, in some instances, the Woodchuck Hepatitis Virus Post-Transcriptional Response Element (WPRE). Vector A is the control IDLV vector. Vector B is the same as vector A except harboring a novel integrase attP target site flanked by universal primer regions U1 and U2 is placed upstream of the transgene. Vector C is the same as vector B except the LTR harboring a deletion in the U3 region.

FIG. 7: Schematic representations of Recombinase-IDLV packaging plasmids. IDLV-recombinase packaging systems include three plasmids: 1. (top) A packaging plasmid expresses the gag-pol gene region of HIV-1 that encodes the enzymatic proteins protease, reverse transcriptase, and integrase (IN), and structural proteins. The D64V mutation is introduced into the catalytic core of HIV integrase (IN) to inhibit integration activity of the enzyme. In this strategy, a recombinase-encoding sequence, fused with a HiBit tag for expression detection, is fused to the N-terminus of the Gag protein, with the linker comprising the protease cleavage site SQNY/PIVQ. 2. (middle) A plasmid expressing REV to facilitate nuclear export of transcripts comprising the cis-acting element RRE. 3. (bottom) An envelope expression plasmid to provide the envelope protein VSV-G. These plasmids are used to package an IDLV vector comprising a DNA recognition sequence into an IDLV viral particle as described in Example 31 or 32, optionally with a recombinase-gag-pol fusion protein. In some embodiments, a recombinase of the system may instead be provided exogenously from the packaging system, e.g., encoded within the IDLV or as an additional nucleic acid provided separately from the system, e.g., as an LNP comprising an mRNA encoding the recombinase.

FIG. 8 is a diagram showing an exemplary IDLV vector system using heterologous integration functions to insert a payload into the genome. An IDLV system as described herein may utilize a DNA recognition sequence comprised by the IDLV and a recombinase (e.g., a recombinase encoded by the packaging system and packaged with the IDLV or a recombinase provided as a separate component, e.g., an mRNA encoding the recombinase) that binds the DNA recognition sequence to facilitate recombinase-mediated integration of an IDLV into a target DNA (e.g., a genomic DNA, e.g., as described herein). In brief, an IDLV comprising a template RNA is delivered to a target cell, reverse transcribed using a reverse transcriptase of the IDLV, and converted to dsDNA. An optional circularization event occurs via an endogenous pathway (e.g., homologous recombination) or an engineered approach (e.g., recombinase or nuclease-mediated cohesive end ligation, as described herein). The DNA recognition sequence of the IDLV (e.g., attP) is recognized by a recombinase enzyme of the system, which facilitates recombination with a genomic DNA target (e.g., attB). Thus, an IDLV-recombinase system can catalyze the integration of a target payload into one or more target sites of the genome.

FIGS. 9A and 9B describe a luciferase activity assay for primary cells. LNPs formulated as according to Example 9 were analyzed for delivery of cargo to primary human (A) and mouse (B) hepatocytes, as according to Example 38. The luciferase assay revealed dose-responsive luciferase activity from cell lysates, indicating successful delivery of RNA to the cells and expression of Firefly luciferase from the mRNA cargo.

FIG. 10 shows LNP-mediated delivery of RNA cargo to the murine liver. Firefly luciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours, post-administration.

FIG. 11 is a schematic representation of lentivirus-attP vectors with or without insulators. The lentivirus vectors shown contain a self-inactivating 3′LTR, a psi sequence (Ψ) allows for efficient incorporation of the vector RNA genome into particles, a Rev responsive element (RRE), a central polypurine tract (cPPT), the expression of EGFP transgene driven by human EF1a promoter, as well as the Woodchuck Hepatitis Virus Post-Transcriptional Response Element (WPRE). Vector A is the control lentivirus vector. Vector B is the same as vector A except that a DNA recognition site (labeled attP) flanked by universal primer regions U1 and U2 is placed upstream of the transgene. Vector C is the same as vector B except the attP site is flanked by insulators.

FIG. 12 is a schematic diagram illustrating insulators flanking a recognition sequence, which result in the insulation of the integrated sequence after recombination. The left panel shows a circular template DNA comprising, from left to right, a first insulator, a DNA recognition sequence, a second insulator, and a heterologous object sequence comprising a promoter and a gene. The right panel shows the template DNA after integration into a host genome, resulting in a sequence comprising, from left to right: host DNA, first recombinase transfer sequence, first insulator, heterologous object sequence comprising a promoter and a gene, second insulator, and second recombinase transfer sequence.

### DETAILED DESCRIPTION

This disclosure relates to compositions, systems and methods for targeting, editing, modifying or manipulating a DNA sequence (e.g., inserting a heterologous object DNA sequence into a target site of a mammalian genome) at one or more locations in a DNA sequence in a cell, tissue or subject, e.g., in vivo or in vitro. The object DNA sequence may include, e.g., a coding sequence, a regulatory sequence, or a gene expression unit.

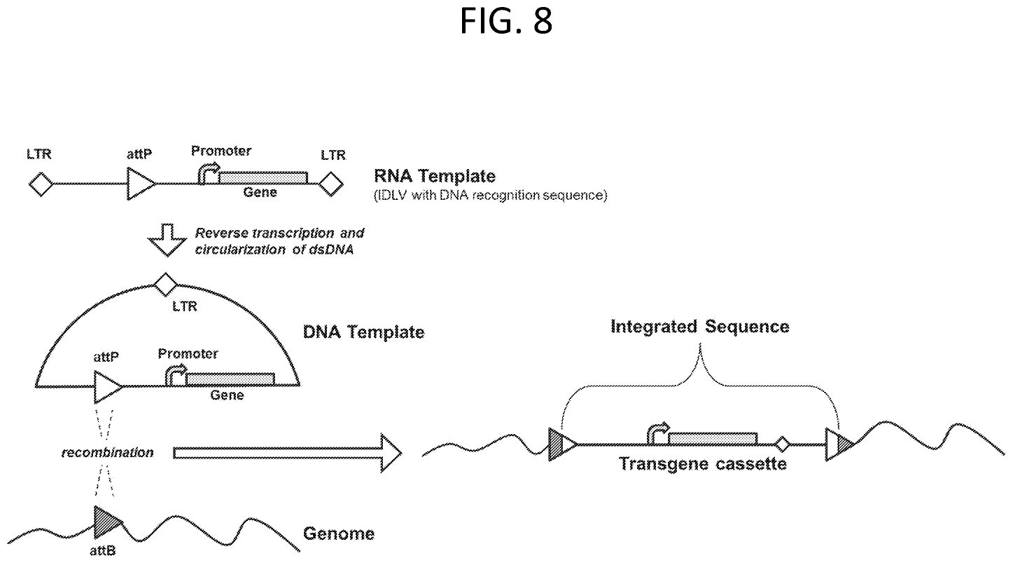

Among other things, provided herein are systems that replace the natural random integration activity of a retrovirus with site-specific integration machinery. This approach allows for a more precise targeting of a gene of interest into a human genome, e.g., for therapeutic purposes. The system may include integration-deficient retrovirus (e.g., lentivirus) (IDLV), in which the natural integration activity has been reduced (e.g., by mutation to the viral integrase polypeptide). Instead, the system may comprise a site-specific recombinase (e.g., a serine recombinase, e.g., a serine integrase) capable of directing insertion of a template DNA, or portion thereof, into a desired site in the human genome. In some embodiments, the recombinase is one that directs insertion into a cognate DNA recognition sequence in a naturally occurring human genome and/or in Genome Reference Consortium Human Build 38. Such a recombinase may advantageously be used in a human cell without the need to engineer the genome to contain a “landing pad” for the recombinase to recognize. The template DNA can comprise a DNA recognition sequence recognized by the site-specific recombinase, which can be recombined with a cognate DNA recognition sequence in the genome. The system can also provide a reverse transcriptase capable of generating a template DNA starting from a template RNA. In some embodiments, a system described herein first reverse transcribes a template DNA from a template RNA, and then second, specifically integrates the template DNA, or a portion thereof, into the genome using site-specific recombinase activity, e.g., as shown in FIG. 8.

Generally, a system as described herein comprises a template RNA, a retroviral (e.g., lentiviral) structural polypeptide domain (or a nucleic acid molecule encoding same), a retroviral (e.g., lentiviral) reverse transcriptase polypeptide domain (or a nucleic acid molecule encoding same), and a recombinase (e.g., a serine recombinase, e.g., a serine integrase, e.g., as described herein) (or a nucleic acid encoding same). The reverse transcriptase polypeptide domain may, in some instances, be capable of reverse transcribing the template RNA to produce a template DNA. The system generally comprises a viral envelope (e.g., a retroviral envelope, e.g., a lentiviral envelope) enclosing the template RNA, structural polypeptide domain, reverse transcriptase polypeptide domain, and/or the recombinase (or the nucleic acid molecule(s) encoding same). Generally, in a system as described herein, the reverse transcriptase polypeptide domain is substantially unable to integrate the template DNA, or a portion thereof, into a target DNA (e.g., a genomic DNA, e.g., a chromosome or a mitochondrial genome), e.g., the reverse transcriptase polypeptide domain is integration-deficient, e.g., as described herein. Generally, the serine recombinase (e.g., serine integrase) is capable of integrating the template DNA, or portion thereof, into the target DNA. In some embodiments, the recombinase is a serine recombinase (e.g., a serine integrase, e.g., as described herein). In some embodiments, the recombinase is a tyrosine recombinase, e.g., as described in PCT Publication No. WO2021/016075 (incorporated herein by reference in its entirety, including the nucleic acid sequences and amino acid sequences of Table 1 and Table 2 therein).

In some embodiments, a serine recombinase as described herein is a large serine recombinase (e.g., a serine recombinase having an amino acid sequence consisting of at least 400 amino acids). In some embodiments, the serine recombinase is at least 400, 450, 500, 550, or 600 amino acids in length. In some embodiments a serine recombinase as described herein is a unidirectional serine recombinase. In some embodiments, a serine recombinase as described herein is a small serine recombinase (e.g., a serine recombinase having an amino acid sequence consisting of less than 400 amino acids). In some embodiments a serine recombinase as described herein is a bidirectional serine recombinase.

Systems as described herein may, in some instances, be IDLV recombinase systems or IDLV attP systems. An IDLV recombinase system as described herein may, in some instances, be referred to as a Gene Writing system. In some instances, the genome of an IDLV is a Gene Writing template, e.g., as described herein. In some instances, a Gene Writing polypeptide (e.g., as described herein) comprises a recombinase (e.g., as described herein), a reverse transcriptase (e.g., as described herein), or a fusion of a recombinase and a reverse transcriptase.

In some embodiments, a Gene Writer system as described herein comprises a template nucleic acid molecule comprising an insulator, a DNA recognition sequence that is specifically bound by a recombinase polypeptide (e.g., a tyrosine recombinase polypeptide or a serine recombinase (e.g., a serine integrase) polypeptide), and a heterologous object sequence. The template nucleic acid molecule may, in some instances, comprise a plurality of insulators (e.g., two insulators). In some instances, the template nucleic acid molecule comprises a first insulator and a second insulator, with the DNA recognition sequence positioned between the first and second insulator. In some instances, recombination of the template nucleic acid molecule with a target DNA (e.g., a genomic DNA, e.g., a chromosome or a mitochondrial genome, e.g., comprising a cognate DNA recognition sequence) by a recombinase polypeptide results in integration of the heterologous object sequence into the target DNA, with the first and second insulators flanking the integrated heterologous object sequence.

### Gene-Writer™ Genome Editors

The present invention provides recombinase polypeptides (e.g., serine recombinase polypeptides, e.g., any of SEQ ID NOs: 1-12,677 (e.g., SEQ ID NOs: 1-11,432)) that can be used to modify or manipulate a DNA sequence, e.g., by recombining two DNA sequences comprising cognate recognition sequences that can be bound by the recombinase polypeptide. A Gene Writer™ gene editor system may, in some embodiments, comprise: (A) a polypeptide or a nucleic acid encoding a polypeptide, wherein the polypeptide comprises (i) a domain that contains recombinase activity, and (ii) a domain that contains DNA binding functionality (e.g., a DNA recognition domain that, for example, binds to or is capable of binding to a recognition sequence, e.g., as described herein); and (B) an insert DNA comprising (i) a sequence that binds the polypeptide (e.g., a recognition sequence as described herein) and, optionally, (ii) an object sequence (e.g., a heterologous object sequence). In some embodiments, the domain that contains recombinase activity and the domain that contains DNA binding functionality is the same domain. For example, the Gene Writer genome editor protein may comprise a DNA-binding domain and a recombinase domain. In certain embodiments, the elements of the Gene Writer™ gene editor polypeptide can be derived from sequences of a recombinase polypeptide (e.g., a serine recombinase), e.g., as described herein, e.g., any of SEQ ID NOs: 1-12,677 (e.g., SEQ ID NOs: 1-11,432). In some embodiments the Gene Writer genome editor is combined with a second polypeptide. In some embodiments the second polypeptide is derived from a recombinase polypeptide (e.g., a serine recombinase), e.g., as described herein, e.g., any of SEQ ID NOs: 1-12,677 (e.g., SEQ ID NOs: 1-11,432).

In some embodiments, a Gene Writer comprises a serine recombinase (e.g., a serine integrase) polypeptide domain comprising the amino acid sequence of a serine recombinase (e.g., a serine integrase) as described in Ioannidi et al. (2021, bioRxiv 2021.11.01.466786; doi: https://doi.org/10.1101/2021.11.01.466786; incorporated herein by reference in its entirety), or an amino acid sequence having at least 75%, 80%, 85%, 90%, 95%, 96%, 97%, 98%, or 99% sequence identity thereto. In some embodiments, a Gene Writer comprises a serine recombinase (e.g., a serine integrase) polypeptide domain comprising the amino acid sequence of a serine recombinase (e.g., a serine integrase) as described in Durrant et al. (2021, bioRxiv preprint doi: https://doi.org/10.1101/2021.11.05.467528; incorporated herein by reference in its entirety), or an amino acid sequence having at least 75%, 80%, 85%, 90%, 95%, 96%, 97%, 98%, or 99% sequence identity thereto. It is understood that, where applicable, any embodiment (e.g., enumerated embodiment) described herein with respect to a serine recombinase polypeptide domain comprising an amino acid sequence of any of SEQ ID NOs: 1-12,677 may instead utilize a serine recombinase as described in this paragraph.

In some embodiments, a Gene Writer comprises one or more components (e.g., nucleic acid molecules or polypeptides) as described in PCT Application No. PCT/US2020/061705 (incorporated by reference herein in its entirety).

### Recombinase Polypeptide Component of Gene Writer Gene Editor System

An exemplary family of recombinase polypeptides that can be used in the systems, cells, and methods described herein includes the serine recombinases. Generally, serine recombinases are enzymes that catalyze site-specific recombination between two recognition sequences. The two recognition sequences may be, e.g., on the same nucleic acid (e.g., DNA) molecule, or may be present in two separate nucleic acid (e.g., DNA) molecules. In some embodiments, a serine recombinase polypeptide comprises a recombinase N-terminal domain (also called the catalytic domain), a recombinase domain, and a C-terminal zinc ribbon domain. In some embodiments the zinc ribbon domain further comprises a coiled-coiled motif. In some embodiments the recombinase domain and the zinc ribbon domain are collectively referred to as the C-terminal domain. In some embodiments the N-terminal domain is between 50 and 250 amino acids, or 100-200 amino acids, or 130-170 amino acids. In some embodiments the C-terminal domain is 200-800 amino acids, or 300-500 amino acids. In some embodiments the recombinase domain is between 50 and 150 amino acids. In some embodiments the zinc ribbon domain is between 30 and 100 amino acids. In some embodiments the N-terminal domain is linked to the recombinase domain via a long helix (sometimes referred to as an uE helix or linker). In some embodiments the recombinase domain and zinc ribbon domain are connected via a short linker. Non-limiting examples of serine recombinases, as well as the recombinase polypeptides, comprising an amino acid sequence of any of SEQ ID NOs: 1-12,677 (e.g., SEQ ID NOs: 1-11,432).

In some embodiments, recombinant recombinases are constructed by swapping domains. In some embodiments, a recombinase N-terminal domain can be paired with a heterologous recombinase C-terminal domain. In some embodiments, a catalytic domain can be paired with a heterologous recombinase domain, zinc ribbon domain, αE helix, and/or short linker. In some embodiments, a C-terminal domain can comprise heterologous recombinase domains, zinc ribbon domains, αE helix, and/or short linkers. In some embodiments, DNA binding elements of the recombinase polypeptide are modified or replaced by heterologous DNA binding elements, such as zinc-finger domains, TAL domains, or Watson-crick based targeting domains, such as CRISPR/Cas systems.

Without wishing to be bound by theory, serine recombinases utilize short, specific DNA sequences (e.g., attP and attB), which are examples of recognition sequences. During the integration reaction, the recombinase binds to attP and attB as a dimer, mediates association of the sites to form a tetrameric synaptic complex, and catalyzes strand exchange to integrate DNA, forming new recognition sequences sites, attL and attR. The new recognition sites, attL and attR, comprises, for example, in order from 5′ to 3′: attB5′-core-attP3′, and attP5′-core-attB3′. Without wishing to be bound by theory, the reverse reaction, where the DNA is excised by site-specific recombination between attL and attR sequences, occurs at reduced frequency or does not occur in the absence of a recombination directionality factor (RDF). This results in stable integration with little or no detectable recombinase-mediated excision, i.e., recombination that is “unidirectional”.

While not wishing to be bound by descriptions of mechanisms, strand exchange catalyzed by recombinases typically occurs in two steps of (1) cleavage and (2) rejoining involving a covalent protein-DNA intermediate formed between the recombinase enzyme and the DNA strand(s). The recombinases act by binding to their DNA substrates as dimers and bring the sites together by protein-protein interactions to form a tetrameric synaptic complex. Activation of the nucleophilic serine in each of the four subunits results in DNA cleavage to give 2 nt 3′ overhangs and transient phosphoseryl bonds to the recessed 5′ ends. DNA strand exchange occurs by subunit rotation. The 3′ dinucleotide overhangs base pair with the recessed 5′ bases and the 3′ OH attacks the phosphoseryl bond in the reverse of the cleavage reaction to join the recombinant half sites. Further details of the structure, activity, and biology of serine recombinases are described in the following references which are incorporated by reference: Smith MCM. 2014. Phage-encoded serine integrases and other large serine recombinases. Microbiol Spectrum 3(4):MDNA3-0059-2014; Rutherford K and Van Duyne G D. 2014. The ins and outs of serine integrase site-specific recombination. Current Opinion in Structural Biology 24: 125-131; Van Duyne G D and Rutherford K. 2013. Large Serine Recombinase domain structure and attachment site binding. Critical Reviews in Biochemistry and Molecular Biology 48(5): 471-491.

A skilled artisan can determine the nucleic acid and corresponding polypeptide sequences of a recombinase polypeptide (e.g., serine recombinase) and domains thereof, e.g., by using routine sequence analysis tools as Basic Local Alignment Search Tool (BLAST) or CD-Search for conserved domain analysis. Other sequence analysis tools are known and can be found, e.g., at https://molbiol-tools.ca, for example, at https://molbiol-tools.ca/Motifs.htm. In some embodiments, a serine recombinase described herein includes at least one known active site signature of a serine recombinase, e.g., cd00338, cd03767, cd03768, cd03769, or cd03770. Proteins containing these domains can additionally be found by searching the domains on protein databases, such as InterPro (Mitchell et al. Nucleic Acids Res 47, D351-360 (2019)), UniProt (The UniProt Consortium Nucleic Acids Res 47, D506-515 (2019)), or the conserved domain database (Lu et al. Nucleic Acids Res 48, D265-268 (2020)), or by scanning open reading frames or all-frame translations of nucleic acid sequences for serine recombinase domains using prediction tools, for example InterProScan.

While the present disclosure provides many particular serine recombinase sequences, it is understood that methods described herein can be performed with other serine recombinases as well. For example, a composition or method described herein may involve a serine recombinase having an active site signature chosen from, e.g., cd00338, cd03767, cd03768, cd03769, or cd03770. In some embodiments, the serine recombinase has a length of above 400 amino acids (e.g., at least 400, 500, 600, 700, 800, 900, or 1000 amino acids). In some embodiments, a recombinase comprises 1, 2, 3, 4, 5, 6, 7, 8, 9, 10, 11, 12, 13, 14, 15, or more domains of any of SEQ ID NOs: 1-12,677 (e.g., SEQ ID NOs: 1-11,432). In some embodiments, a recombinase comprises 1, 2, 3, 4, 5, 6, 7, 8, 9, 10, 11, 12, 13, 14, 15, or more domains listed in Table 1. In some embodiments, a method for identifying a recombinase comprises determining whether a polypeptide comprises 1, 2, 3, 4, 5, 6, 7, 8, 9, 10, 11, 12, 13, 14, 15, or more domains of any of SEQ ID NOs: 1-12,677 (e.g., SEQ ID NOs: 1-11,432). In some embodiments, a method for identifying a recombinase comprises determining whether a polypeptide comprises 1, 2, 3, 4, 5, 6, 7, 8, 9, 10, 11, 12, 13, 14, 15, or more domains listed in Table 1.

### Exemplary Recombinase Polypeptides

In some embodiments, a Gene Writer™ gene editor system comprises a recombinase polypeptide (e.g., a serine recombinase polypeptide), e.g., as described herein. Generally, a recombinase polypeptide (e.g., a serine recombinase polypeptide) specifically binds to a nucleic acid recognition sequence and catalyzes a recombination reaction at a site within the recognition sequence (e.g., a core sequence within the recognition sequence). In some embodiments, a recombinase polypeptide catalyzes recombination between a recognition sequence, or a portion thereof (e.g., a core sequence thereof) and another nucleic acid sequence (e.g., an insert DNA comprising a cognate recognition sequence and, optionally, an object sequence, e.g., a heterologous object sequence). For example, a recombinase polypeptide (e.g., a serine recombinase polypeptide) may catalyze a recombination reaction that results in insertion of an object sequence, or a portion thereof, into another nucleic acid molecule (e.g., a genomic DNA molecule, e.g., a chromosome or mitochondrial DNA).

The sequence listing, e.g., in SEQ ID NOs: 1-12,677 (e.g., SEQ ID NOs: 1-11,432), provides amino acid sequences of exemplary recombinase polypeptides, e.g., serine recombinases (e.g., serine integrases), or fragments thereof. The sequence listing, e.g., in SEQ ID NOs: 13,001-25,677 or SEQ ID NOs: 26,001-38,677, further provides exemplary flanking nucleic acid sequences of the nucleic acid sequence encoding the exemplary serine recombinase in the organism of origin (e.g., SEQ ID NOs: 13,001-25,677 (e.g., SEQ ID NOs: 13,001-24,432) or SEQ ID NOs: 26,001-38,677 (e.g., SEQ ID NOs: 26,001-37,432) representing LeftRegion and RightRegion, respectively); one or both of these flanking nucleic acid sequences comprise the native recognition sequence or the portions thereof (e.g., comprise an attP site or portions thereof) of the corresponding recombinase. The terms “LeftRegion” and “RightRegion” do not imply any particular placement or directionality. Without wishing to be bound by theory, a given set of LeftRegion and RightRegion sequences may be positioned on either end of a nucleic acid sequence of interest (e.g., a nucleic acid sequence encoding an exemplary serine recombinase, e.g., in a bacterial genome). For example, in some embodiments, the LeftRegion is located upstream (e.g., 5′) relative to the nucleic acid sequence of interest (e.g., a coding region in the nucleic acid sequence of interest). In some embodiments, the LeftRegion is located downstream (e.g., 3′) relative to the nucleic acid sequence of interest (e.g., a coding region in the nucleic acid sequence of interest). In some embodiments, the RightRegion is located upstream (e.g., 5′) relative to the nucleic acid sequence of interest (e.g., a coding region in the nucleic acid sequence of interest). In some embodiments, the RightRegion is located downstream (e.g., 3′) relative to the nucleic acid sequence of interest (e.g., a coding region in the nucleic acid sequence of interest). SEQ ID NOs: 1-11,432 comprise amino acid sequences that had not previously been identified as serine recombinases, and SEQ ID NOs: 13,001-24,432 or SEQ ID NOs: 26,001-37,432 comprise corresponding flanking nucleic acid sequences (and thereby DNA recognition sequences) of serine recombinases for which the DNA recognition sequences were previously unknown. Domains identified as present in the exemplary recombinase sequences are also identified based on InterPro analysis of the amino acid sequence (see corresponding descriptive field in the sequence listing). See, e.g., https://omictools.com/interpro-tool. A brief key to the domain nomenclature is provided in Table 1.

In some embodiments, a recombinase polypeptide described herein comprises one or more domains listed in Table 1. In some embodiments, a recombinase polypeptide described herein comprises one or more (e.g., 2, 3, 4, or all) of the domains listed in the corresponding descriptive field for that polypeptide sequence in the sequence listing. In some embodiments, a recombinase polypeptide described herein comprises one or more (e.g., 2, 3, 4, or all) of the domains listed in the corresponding descriptive field for any of SEQ ID NOs: 1-12,677.

Each of the native recognition sequences or portions thereof occurring in the flanking nucleic acid sequences of any of SEQ ID NOs: 13,001-25,677 (e.g., SEQ ID NOs: 13,001-24,432) or SEQ ID NOs: 26,001-38,677 (e.g., SEQ ID NOs: 26,001-37,432) may comprise one, two, or three of: (i) a first parapalindromic sequence, (ii) a core sequence, and/or (iii) a second parapalindromic sequence, wherein the first and second parapalindromic sequences are parapalindromic relative to each other.

TABLE 1
Exemplary integrase domains

| domainID | Description |
|---|---|
| PF07508 | Recombinase, usually found with PF00239 |
| G3DSA: | Putative LSR |
| 1.10.10.2600 | |
| SSF53041 | Resolvase like |
| PF13408 | Recombinase zinc ribbon |
| PS51737 | DNA-binding recombinase domain profile |
| cd00338 | Serine Recombinase family, catalytic domain; a |
| | DNA binding domain may be pres . . . |
| cd03770 | Serine Recombinase (SR) family, TndX-like |
| | transposase subfamily, catalytic do. |
| PF14287 | Domain of unknown function |
| PTHR30461: SF21 | Subfamily not named |
| cd03768 | Serine Recombinase (SR) family, Resolvase and |
| | Invertase subfamily, catalytic |
| PTHR30461: SF3 | SERINE RECOMBINASE PINE-RELATED |
| PF00239 | Resolvase, N-terminal |
| PS51736 | Resolvase/invertase-type recombinase catalytic |
| | domain profile |
| SM00857 | Resolvase, N terminal domain |
| G3DSA: | Resolvase, N-terminal |
| 3.40.50.1390 | |
| PS00397 | Site-specific recombinases active site |
| PS00398 | Site-specific recombinases signature 2 |
| cd00569 | Helix-turn-helix domain of Hin and related proteins |
| PTHR30461 | DNA-INVERTASE FROM LAMBDOID |
| | PROPHAGE |
| G3DSA: | Homeodomain-like |
| 1.10.10.60 | |
| SSF46689 | Homeodomain-like |
| PF02796 | Helix-turn-helix domain of resolvase |

TABLE 2
Exemplary recombinase recognition sites

| Inte- | | | |
|---|---|---|---|
| grase | Protein | | |
| A No. | Accession | attB | attP |
| 23 | YP_006907 | GATCAGCTCCGCGGGCAAGACCTTTC | TCCAGCCCAACAGTGTTAGTCTTTGC |
| | 228.1 | TCCTTCACGGGGTGGAAGGTC | TCTTACCCAGTTGGGCGGGA |
| 25 | NP_047974 | TGCGGGTGCCAGGGCGTGCCCTTGGG | GTGCCCCAACTGGGGTAACCTTTGAG |
| | .1 | CTCCCCGGGCGCGTACTCC | TTCTCTCAGTTGGGGG |
| 27 | NP_813744 | CAGGTTTTTGACGAAAGTGATCCAGA | GGTGCTGGGTTGTTGTCTCTGGACAG |
| | .2 | TGATCCAG | TGATCCATGGGAAACTACTCAGCACC |
| 56 | AFU62167. | CATCAGGGCGGTCAGGCCGTAGATGT | ATGTGGTCCTTTAGATCCACTGACGT |
| | 1 | GGAAGAAACGGCAGCACGGCGAGGAC | GGGTCAGTGTCTCTAAAGGACTCGCG |
| | | G | |
| 81 | APC43293.1 | ATCTGGATGTGGGTGTCCATCTGCGG | AGTTGTGGCCATGTGTCCATCTGGGG |
| | | GCAGACGCCGCAGTCGAAGCACGG | GCAGATGGAGACGGGGTCACA |
| 111 | NP_817623 | CGGTCTCCATCGGGATCTGCTGATCG | TAACCGCAAGTGTACATCCCTCGGCT |
| | .1 | AGCAGCATGCCGACCA | GGCCGAGACAAGTACAGTTGCGACAG |
| 165 | YP_003358 | CGGTCTCCATCGGGATCTGCTGATCG | TAGTTTCCAATGTTACAGGAACTGCT |
| | 736.1 | AGCAGCATGCCGACCA | GGCAGAATCCAACACATTGGAAGTCG |
| 204 | NP_075302 | GGCTTGTCGACGACGGCGGTCTCCGT | GGTTTGTCTGGTCAACCACCGCGGTC |
| | .1 | CGTCAGGATCAT | TCAGTGGTGTACGGTACAAACC |
| 329 | AAK38018. | ATGCCAACACAATTAACATCTCAATC | GAGTTTTTATTTCGTTTATTTCAATT |
| | 1 | AAGGTAAATGCTTTTTGCTTTTTTTG | AAGGTAACTAAAAAACTCCTTTTAAG |
| | | C | G |
| 414 | YP_006990 | GCATGTTCCCCAAAGCGATACCACTT | TGTTCCCCAAAGCGATACCACTTGAA |
| | 167.1 | GAAGCAGTGGTACTGCTTGTGGGTAC | GCAGTGGTACTGCTTGTGGGTACA |
| | | ACTCTGCGGGTG | |
| 436 | YP_009103 | CAAAGGATCACTGAATCAAAAGTATT | ATTTTAGGTATATGATTTTGTTTATT |
| | 095.1 | GCTCATCCACGCGAAA | AGTGTAAATAACACTATGTACCTAAA |
| | | | AT |
| 450 | CAR95427. | GTAGATTTGTTTCCCCAGACGCACAC | TATTAGTATAGAAGAAAGCTCTCAGC |
| | 1 | GTGGAGTGTGTAAGTTTACTTGAGAA | ACACGTGGAGTGTGTTGCTCTCTGCT |
| | | ACGGAGTTAA | CGTAAAGCCT |
| 477 | NP_463492 | TTTCGGATCAAGCTATGAAGGACGCA | TTCCTCGTTTTCTCTCGTTGGAAGAA |
| | .1 | AAGAGGGAACTAAA | GAAGAAACGAGAAA |
| 524 | AYN56706. | AAGTGTCCAAGCTGGCCCCCGATCCC | TATAATTTCGTATATTAGATATAACC |
| | 1 | AGTTTCAATAGTTTGGGGAATCTTTG | GGTTTCAATTGGAAATACCTAATATA |
| | | TAAGTGGTAA | CGAAAAAAGG |
| 1116 | WP_12913 | CACCCATTGTGTTCACAGGAGATACA | TGTAGTAAGTATCTTAATATACAGCT |
| | 7749.1 | GCTTTATCTGTACTGATATTAATGAC | TTATCTGTTTTTTAAGATACTTACTA |
| | | ATGCTG | CTTT |
| 1200 | YP_459991. | CGGAAGGTAGCGTCAACGATAGGTGT | CTAGTTTTAAAGTTGGTTATTAGTTA |
| | 1 | AACTGTCGTGTTTGTAACGGTACTTC | CTGTGATATTTATCACGGTACCCAAT |
| | | CAACAGCTGGCGCCGCCAC | AACCAATGAAT |
| 1203 | YP_006082 | CTTCCAGCACATCACCCACATGGTCT | GGTATTGTATCAATTTCAGAACTCAC |
| | 695.1 | GTGTCGGTGTGCGTCAGCACTAGACT | ACTTCGGTATGCGTACTCAATTTGAT |
| | | ATCAATCCTA | ACAATTACAA |
| 1204 | YP_005549 | TTTTTTCCGCCTGTCGTAACCGGATC | CTTTTTTGTTGTACTTAAACAATAAT |
| | 228.1 | TGTTGTAACGATTATCGGAATGACCT | GCTTGTAAGAATTATTGATTGAGTAC |
| | | TGATGCCGG | GACATAAACC |
| 1205 | YP_189066. | ACTTCCAATTAACCCTTCACCAGCCC | TTATATTTCGACTTAATTAAGTACAG |
| | 1 | TATACCAAGTTCCTGTCGCGCATCCT | TTTCCACCTAGAGATAGACAAATAAA |
| | | CCAGCTAAT | GTATTATTA |
| 1206 | YP_005679 | ACTACTTAATATATCCATAAGAGAAA | GTTAGGTGTATATCATACCTAACGCA |
| | 179.1 | TTTCATTTCCTTCTTTGTCTACCCCT | ATTCATTACATCACATATGTTATACA |
| | | ATAGGATCTT | CCTACTTTAA |
| 1207 | YP_002804 | ACGAAATAAAAGATTGTATAGATGCT | AAAAGAATCCAAATTATCGTACTTTA |
| | 732.1 | GGTAGGAAACATGCCCTTGTCATTTA | ACATAGTGAATACTGTCCATCATGTA |
| | | GCTGAAACAG | TAAAAGTACG |
| 1208 | YP_001089 | TATTCTAAGTAATGTAGTTTTACCAC | TATATAATTATTTGGACTAACATATA |
| | 468.1 | ATCCACTAGGTCCGAGTAAACATAGA | GTATCCACTTGGCTATTATTAGTTAG |
| | | AATTCCCCT | TCCAAATAAATA |
| 1209 | YP_001886 | ATGGATTTTGCAGATTCCCAGATGCC | GTTTATATGTTTACTAATAAGACGCT |
| | 479.1 | CCTACAGAAAGAGGTACAAAACATTT | CTCAACCCATAAAGTCTTATTAGTAA |
| | | ATTGGAATTAATT | ACATATTTCAACT |
| 1211 | YP_005759 | GTTCGTGGTAACTATGGGTGGTACAG | TTTTTGTATGTTAGTTGTGTCACTGG |
| | 947.1 | GTGCCACATTAGTTGTACCATTTATG | GTAGACCTAAATAGTGACACAACTGC |
| | | TTTATGTGGTTAAC | TATTAAAATTTAA |
| 1212 | YP_004586 | CAAGAAACGTTCCGTCTGTTTGTGTC | TTATAAACCTGTTTTAAAGTTAACTT |
| | 821.1 | AGCTGCGCGAAATTAATGACCGGATC | TACATGCCTAACATTAACTCTTATAC |
| | | GTTTGTTCC | AGGTTAAGGT |
| 1213 | YP_353073. | GGAACTCCGCCGGGCCCATCTGGTCG | ATGGGGTCACAATACCAATCATGTTC |
| | 2 | AAGAAGATGAAGGGGCCCACCATCTG | AAGAATGTGAAGGGTATTTTACCCTT |
| | | CCTCCGGGCC | GTCGTTTCAG |
| 1214 | BAG46462. | GATACGGATGTTCGTCGCCGGCACGC | AGTTGTCTGATAATATATTTTCGGAC |
| | 1 | TGGTCACGCTCGGCAATCCCAAGATC | ACGCTCGGCAACCCGAACGAGAGTCA |
| | | ATGCTGTTCT | AAATACATTT |
| 1217 | SGE40566.1 | GAAGGTGTTGGTGCGGGGTTGGCCGT | GTAGTGTATCTCACAGGTCCACGGTT |
| | | GGTCGAGGTGGGGT | GGCCGTGGACTGCTGAAGAACATTCC |
| 1218 | CBG73463. | GGACGGCGCAGAAGGGGAGTAGCTCT | GCTCATGTATGTGTCTACGCGAGATT |
| | 1 | TCGCCGGACCGTCGACATACTGCTCA | CTCGCCCGAGAACTTCTGCAAGGCAC |
| | | GCTCGTC | TGCTCTTGGCT |
| 1219 | YP_001376 | GCATACATTGTTGTTGTTTTTCCAGA | CAATAACGGTTGTATTTGTAGAACTT |
| | 196.1 | TCCAGTTGGTCCTGTAAATATAAGCA | GACCAGTTGTTTTAGTAACATAAATA |
| | | ATCCATGTGAGT | CAACTCCGAATA |
| 1221 | CAC97653.1 | TAACTTTTTCGGATCGAGTTATGATG | GTTTAGTATCTCGTTATCTCTCGTTG |
| | | GACGTAAAGAGGGAACAAAGCATCTA | GAGGGAGAAGAAACGGGATACCAAAA |
| 1222 | CAD10281. | TTTCGGATCAAGCTATGAAGGACGCA | TTCCTCGTTTTCTCTCGTTGGACGGA |
| | 2 | AAGAGGGAACTAAA | AACGAATCGAGAAA |
| 1223 | YP_004301 | TTTCCACAGACAACTCACGTGGAGGT | CAATGAAAAACTAGGCATGTAGAAGT |
| | 563.1 | AGTCAC | TGTTTGT |
| 1224 | YP_006538 | TTCTGGACCATGATGCGCCACTTCCG | GTATCTTGATGTACAACATTACTCTT |
| | 656.1 | AAATTTCAAAAAGATCAGTGGTCAAA | TATTTTCAAATACAGAATAATGTTGC |
| | | CGGCTCATTA | ATATAATATT |
| 1225 | YP_006685 | CTGTAACTTTTTCGGATCAAGCTATG | TTGTTTAGTCCCTCGTTTTCTCTCGT |
| | 721.1 | AGGGACGCAAAGAGGGAACTAAACAC | TGGACGGAGACGAATCGAGAAACTAA |
| | | TTAATTGGTG | AATTATAAAT |
| 1226 | YP_001384 | TATTCAATTATGTGTCGTAATTTTTA | TATATACTTATAGATACTAAATATTT |
| | 783.1 | TCTATTGCGACGAAAAAACACCATAA | TTGTATTGCGTAACTTCTTCTACACC |
| | | AATTCTAAC | TGTAATATCT |
| 1227 | YP_001392 | AGAAATAGACCTTTCAACTGGACAAG | AAATATAACCTGTGTATTGAAACAAG |
| | 519.1 | GTGCTGATAAAACTATGCAGCAAGTC | GTGCTGATAAAACCCTTTCATAAACA |
| | | TTAAGTAAA | CAAGTAAATA |
| 1228 | BAF67264.1 | TTTATATTGCGAAAAATAATTGGCGA | GTGGTTGTTTTTGTTGGAAGTGTGTA |
| | | ACGAGGTAACTGGATACCTCATCCGC | ATCAGGTATCTGCATAGTTTTCCGAA |
| | | CAATTAAAATTTG | CTTCCAATTA |
| 1229 | NP_470568 | TTATTGCAAGAAAAATGGGTTATAAG | TTATATAAAATAGTGTTTTTGTAAAG |
| | .1 | TACACATCAGGTTATAGTAATATCGA | TACACATCACCATATTTGACAAAAAA |
| | | AAAAGGAAGC | CCTATAAATA |
| 1230 | YP_706485. | ATCGCGCAGAACGGTGCGGTGATCAG | CTATGTGGTGGTAATAGCGAGTAGGG |
| | 1 | TGAGTACGCACCGGGCACGACACCGG | GACTACTCGCTCCAGGTACATTAACA |
| | | CGAAGCATCG | CCATGGA |
| 1231 | YP_002336 | GTAATATGTTTGGATATGGGGAAGTG | ATAATAGTGTATATGGTAGAGAATTA |
| | 631.1 | AATCAGTACAACCGCCACAGTACCCT | CAACCAGTTTAATACTCCACATGTAC |
| | | CATGTCAGCC | ACGCAGTGAG |
| 1232 | YP_001646 | TTTGTAGCCATTAGGCGCATTAGGTT | CGTCACCTTGTTGGCGTAATTAGATT |
| | 422.1 | GACGCCATTAAGCCCTAAAGCATCAT | TACTCCAACAGGGTGATGACAAAGCT |
| | | TCGTCGAAAC | AATGAATTTT |
| 1233 | NP_268897 | GTTTGTAAAGGAGACTGATAATGGCA | ATGGATAAAAAAATACAGCGTTTTTC |
| | .1 | TGTACAACTATACTCGTCGGTAAAAA | ATGTACAACTATACTAGTTGTAGTGC |
| | | GGCATCTTAT | CTAAATAATGCTT |
| 1234 | YP_005869 | AATACTAATAATAGCTAGTACAATTA | CGCTTAATTGCGAGTTTTTATTTCGC |
| | 510.1 | ACATCTCTATCAAAGTAAAAGCTTTT | TTATCTCAATTAAGGTAACTAAAAAA |
| | | AGCTCTTTCT | CTCCTTTT |
| 1235 | YP_002736 | TCTGGTGTAGACGTTAAACGTCCAAT | TATTTCTGTATTTTAGTCAAAGTAAT |
| | 920.1 | CAAGATAACTTTATTATACATATTTT | TAAGATAAGTTAGAGTTAGTAACAGT |
| | | CTTCCTCCTA | ATTTTAACTT |
| 1236 | YP_003445 | TAGGAGGAAAAAATATGTATAATAAA | GTTAATAATATGTATTTAAGTCTAAC |
| | 547.1 | GTTATCATGATTGGGCGTTTGACGTC | TTATCATGACAAATTTGACTAAAATA |
| | | TACACCAGAAT | CAAAAAGGC |
| 1238 | YP_002747 | TTCCAAAGAGCGCCCAACGCGACCTG | AAAAATTACAAAGTTTTCAACCCTTG |
| | 001.1 | AAATTTGAATAAGACTGCTGCTTGTG | ATTTGAATTAGCGGTCAAATAATTTG |
| | | TAAAGGCGATGATT | TAATTCGTTT |
| 1239 | BAE05705.1 | CAATCATCAGATAACTATGGCGGCAC | TTAATAAACTATGGAAGTATGTACAG |
| | | GTGCATTAACCACGGTTGTATCCCGT | TCTTGCAATGTTGAGTGAACAAACTT |
| | | CTAAAGTACTCGT | CCATAATAAAAT |
| 1240 | YP_003472 | AATCTGCAAACATGTATGGCGGTACA | ATTTTTGTACGGAAGTAGATACTATC |
| | 505.1 | TGTATCAACATTGGTTGTATTCCTAC | TTTCAATATCCATGTTACTTAGTGCC |
| | | AAAGACACTCAT | ATACAAAAA |
| 1241 | BAF92844.1 | CGAAAATGTATGGAGGCACTTGTATC | TTTGTGCGGAACTACGAACAGTTCAT |
| | | AATATAGGATGTATACCTTCGAAGAC | TAATACGAAGTGTACAAACTTCCATA |
| | | ACTT | CAA |
| 1242 | YP_003251 | AGACGAGAAACGTTCCGTCCGTCTGG | GTGTTATAAACCTGTGTGAGAGTTAA |
| | 752.1 | GTCAGTTGGGCAAAGTTGATGACCGG | GTTTACATGCCTAACCTTAACTTTTA |
| | | GTCGTCCGTT | CGCAGGTTCAGCTT |
| 1244 | YP_003880 | AGCACGCTGATAATCAGCAAGACCAC | GGAAAATATAAATAATTTTAGTAACC |
| | 342.1 | CAACATTTCCACCAATGTAAAAGCTT | TACATCTCAATCAAGGATAGTAAAAC |
| | | TAACCTTAGC | TCTCACTCTT |
| 1245 | BAF03598.1 | GAGCGCCGGATCAGGGAGTGGACGGC | CCCTAATACGCAAGTCGATAACTCTC |
| | | CTGGGAGCGCTACACGCTGTGGCTGC | CTGGGAGCGTTGACAACTTGCGCACC |
| | | GGTCGGTGC | CTGATCTG |

In some embodiments, a sequence comprising the LeftRegion nucleic acid sequence of SEQ ID NO: 24,761) comprises the nucleic acid sequence:

TCAAAGGTTGATGTTACTGCTGATAATGTAGATATCATATTTAAATT
CCAACTCGCTTAATTGCGAGTTTTTATTTCGTTTATTTCAATTAAGG
TAACTAAAAAACTCCTTTTAAGGAGTTTCTGTAATCAATTAATTTCT
TCAATATATTTTATTTGGTCCCATAGTTCATCAGTTATCTCATGCAT
AGAAGGTTTTTGTTTTGTTTGTATTAGATATCCTTTCTCCTTAAGCA
TGTTAACTACTTTCTTTAGTTTCTG.

In some embodiments, a sequence comprising the LeftRegion nucleic acid sequence of SEQ ID NO: 24,956) comprises the nucleic acid sequence:

TTAATTAAAAAAATAGACGTATGGAACGATAATAAAATTAAGATCC
ACTGGAATATTTAATTTTTTAGGCGCTTTACGCCTTTTTTCGTATA
TTAGGTATTTCCAATTGAAACCGGTTATATCTAATATACGAAATTA
TACAACAAAAAGCCCCAGTGACCATTGCATAATCTGCAACAACCAC
TAGGGCTAAATTTTTATTGACGTTGTGAGTAAACAACTGAATTGAG
TTGCTGTTGGTTAACACCATTGGCAATATC.

In some embodiments, a recombinase recognition site (e.g., as described herein) comprises an attB sequence. In some embodiments, a recombinase recognition site (e.g., as described herein) comprises an attP sequence. In some embodiments, a recombinase recognition site (e.g., as described herein) comprises an attB sequence and an attP sequence. In embodiments, the attB sequence is selected from a sequence listed in Table 2. In embodiments, the attP sequence is selected from a sequence listed in Table 2. In some embodiments, a recombinase recognition site (e.g., as described herein) comprises an attB sequence and an attP sequence, wherein the attB and attP sequences each comprise a sequence as listed in a single row of Table 2.

In some embodiments, a DNA recognition sequence (e.g., as described herein) comprises an attB sequence. In some embodiments, a DNA recognition sequence (e.g., as described herein) comprises an attP sequence. In some embodiments, a DNA recognition sequence (e.g., as described herein) comprises an attB sequence and an attP sequence. In embodiments, the attB sequence is selected from a sequence listed in Table 2. In embodiments, the attP sequence is selected from a sequence listed in Table 2. In some embodiments, a DNA recognition sequence (e.g., as described herein) comprises an attB sequence and an attP sequence, wherein the attB and attP sequences each comprise a sequence as listed in a single row of Table 2.

In some embodiments, a recombinase polypeptide (e.g., comprised in a system or cell as described herein) comprises an amino acid sequence of any of SEQ ID NOs: 1-12,677 (e.g., any of SEQ ID NOs: 1-11,432), or an amino acid sequence having at least 70%, 75%, 80%, 85%, 90%, 95%, 96%, 97%, 98%, 99%, or 100% sequence identity thereto. In some embodiments, a recombinase polypeptide (e.g., comprised in a system or cell as described herein), or a portion thereof, has at least 70%, 75%, 80%, 85%, 90%, 95%, 96%, 97%, 98%, 99%, or 100% sequence identity to the amino acid sequence of a recombinase domain, a DNA recognition domain (e.g., that binds to or is capable of binding to a recognition site, e.g. as described herein), a recombinase N-terminal domain (also called the catalytic domain), a zinc ribbon domain, the coiled coil motif of a zinc ribbon domain, or a C-terminal domain (e.g., the recombinase domain and the zinc ribbon domain) of a recombinase polypeptide of any of SEQ ID NOs: 1-12,677 (e.g., any of SEQ ID NOs: 1-11,432). In some embodiments, a recombinase polypeptide (e.g., comprised in a system or cell as described herein) has one or more of the DNA binding activity and/or the recombinase activity of a recombinase polypeptide comprising an amino acid sequence of any of SEQ ID NOs: 1-12,677 (e.g., any of SEQ ID NOs: 1-11,432), or an amino acid sequence having at least 70%, 75%, 80%, 85%, 90%, 95%, 96%, 97%, 98%, 99%, or 100% sequence identity thereto.

In some embodiments, an insert DNA (e.g., comprised in a system or cell as described herein) comprises a nucleic acid recognition sequence occurring within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleic acid sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto. In some embodiments, an insert DNA (e.g., comprised in a system or cell as described herein) comprises one or more (e.g., both) parapalindromic sequences occurring within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleic acid sequence having at least 70%, 75%, 80%, 85%, 90%, 95%, 96%, 97%, 98%, or 99% identity to said parapalindromic sequence, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto. In some embodiments, an insert DNA (e.g., comprised in a system or cell as described herein) comprises a spacer (e.g., a core sequence) of a nucleic acid recognition sequence occurring within a nucleotide sequence in the of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleic acid sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto. In certain embodiments, the insert DNA further comprises a heterologous object sequence.

In some embodiments, an insert DNA (e.g., comprised in a system or cell as described herein) comprises a nucleic acid recognition sequence occurring within a nucleotide sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a nucleic acid sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto, that is the cognate to a pseudo-recognition sequence (e.g., a human recognition sequence).

In some embodiments, an insert DNA or recombinase polypeptide used in a composition or method described herein directs insertion of a heterologous object sequence into a position having a safe harbor score of at least 3, 4, 5, 6, 7, or 8.

In certain embodiments, recombination between the insert DNA and the human DNA recognition sequence results in the formation of an integrated nucleic acid molecule comprising two recognition sequences flanking the integrated sequence (e.g., the heterologous object sequence). Without wishing to be bound by theory, serine recombinases facilitate recombination between recognition sequences comprising attB and attP sites and by recombination form recognition sequences comprising attL and attR sites, e.g., flanking the integrated sequence. While a serine recombinase may recognize, e.g., bind, to an attL or attR site, the serine recombinase will not appreciably (e.g., will not) facilitate recombination using the attL or attR sites (e.g., in the absence of an additional factor). The attL and attR sites comprise recombined portions of the attP and attB sites from which they were created. In certain embodiments, one or both of the two post-recombination recognition sequences of the integrated nucleic acid molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more mismatches as compared to one or more of (e.g., one, two, or all three of): (i) the native recognition sequence, (ii) the recognition sequence on the insert DNA, and/or (iii) a pseudo-recognition sequence (e.g., a human DNA recognition sequence). In embodiments, one or both of the two post-recombination recognition sequences of the integrated nucleic acid molecule comprises 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, or more mismatches as compared to the native recognition sequence. In some embodiments the mismatches are present in the core sequence. It is contemplated that, in some embodiments, these differences between the recognition sequence(s) of the integrated nucleic acid molecule and the native recognition sequence, the insert DNA recognition sequence, and/or the human DNA recognition sequence result in reduced binding affinity between the recombinase polypeptide and the recognition sequences of the integrated nucleic acid molecule and/or reduced (e.g., eliminated) recombinase activity of the recombinase polypeptide on the recognition sequences of the integrated nucleic acid molecule, compared to the binding and/or activity of the recombinase to the recognition sequence(s) the native recognition sequence, the insert DNA recognition sequence, and/or the human DNA recognition sequence.

In some embodiments, a pseudo-recognition sequence (e.g., a human DNA recognition sequence) is located in or near (e.g., within 1, 2, 3, 4, 5, 10, 15, 20, 30, 40, 50, 75, 100, 150, 200, 300, 400, 500, 600, 700, 800, 900, 1000, 2000, 3000, 4000, 5000, or 10,000 nucleotides of) a genomic safe harbor site. In some embodiments, the pseudo-recognition sequence (e.g., human recognition sequence) is located at a position in the genome that meets 1, 2, 3, 4, 5, 6, 7, 8 or 9 of the following criteria: (i) is located >300 kb from a cancer-related gene; (ii) is >300 kb from a miRNA/other functional small RNA; (iii) is >50 kb from a 5′ gene end; (iv) is >50 kb from a replication origin; (v) is >50 kb away from any ultraconserved element; (vi) has low transcriptional activity (i.e. no mRNA+/−25 kb); (vii) is not in a copy number variable region; (viii) is in open chromatin; and/or (ix) is unique, with 1 copy in the human genome.

In embodiments, a cell or system as described herein comprises one or more of (e.g., 1, 2, or 3 of): (i) a recombinase polypeptide comprising an amino acid sequence of SEQ ID NO: n (where n is chosen from 1-12,677 (e.g., 1-11,342)), or an amino acid sequence having at least 70%, 75%, 80%, 85%, 90%, 95%, 96%, 97%, 98%, 99%, or 100% sequence identity thereto; (ii) an insert DNA comprising a DNA recognition sequence occurring within a nucleotide sequence corresponding to a) a LeftRegion comprising a nucleotide sequence according to SEQ ID NO: (n+13,000), b) a RightRegion comprising a nucleotide sequence according to SEQ ID NO: (n+26,000), or both a) and b), or a nucleic acid sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto, optionally wherein the insert DNA further comprises an object sequence (e.g., a heterologous object sequence); and/or (iii) a genome comprising a pseudo-recognition sequence (e.g., a human recognition sequence) sequence corresponding to a) a LeftRegion comprising a nucleotide sequence according to SEQ ID NO: (n+13,000), b) a RightRegion comprising a nucleotide sequence according to SEQ ID NO: (n+26,000), or both a) and b), or a nucleic acid sequence having at least 70%, 75%, 80%, 85%, 90%, 95%, 96%, 97%, 98%, or 99% identity thereto, or having no more than 1, 2, 3, 4, 5, 6, 7, 8, 9, 10, 11, 12, 13, 14, 15, 16, 17, 18, 19, or 20 sequence alterations (e.g., substitutions, insertions, or deletions) relative thereto.

In some embodiments, a recombinase recognition site, e.g., an attB, attP, attL, or attR site, can be predicted by available software tools. In some embodiments, the recognition sites may be predictable by a phage prediction tool, e.g., PhiSpy (Akhter et al. Nucleic Acids Res 40(16):e126 (2012)) or PHASTER (Arndt et al. Nucleic Acids Res 44:W16-W21 (2016)), incorporated herein by reference. In some embodiments, the region proximal to an integrase coding sequence in its native context, e.g., in a bacteriophage genome, plasmid, or bacterial genome, e.g., any of SEQ ID NOs: 13,001-25,677 (e.g., SEQ ID NOs: 13,001-24,432) or SEQ ID NOs: 26,001-38,677 (e.g., SEQ ID NOs: 26,001-37,432), comprises the native attachment site of a recombinase enzyme. In some embodiments, a minimal attachment site can be discovered empirically by testing fragments of the integrase proximal sequence, e.g., any of SEQ ID NOs: 13,001-25,677 (e.g., SEQ ID NOs: 13,001-24,432) or SEQ ID NOs: 26,001-38,677 (e.g., SEQ ID NOs: 26,001-37,432), until the minimal sequence sufficient for a productive recombination reaction is discovered. In some embodiments, an integrase proximal sequence, e.g., any of SEQ ID NOs: 13,001-25,677 (e.g., SEQ ID NOs: 13,001-24,432) or SEQ ID NOs: 26,001-38,677 (e.g., SEQ ID NOs: 26,001-37,432), or a fragment thereof, is assayed to determine the importance of each nucleotide, e.g., is profiled in a library format as per the methods of Bessen et al. Nat Commun 10:1937 (2019), incorporated herein by reference in its entirety. In some embodiments, a recombinase or a recombinase recognition site is selected through an evolutionary process for altered protein-nucleic acid interaction properties, e.g., a recombinase used in a Gene Writer system is evolved as described in WO2017015545, incorporated herein by reference in its entirety. In some embodiments, a recombinase and/or a recombinase recognition site is discovered through prediction of the ends of an integrated element in a native host genome, e.g., an integrated bacteriophage or integrated plasmid, e.g., as described in Yang et al. Nat Methods 11(12):1261-1266 (2014), incorporated herein by reference in its entirety.

In some embodiments, an attL or attR site is present in the human genome and the template DNA comprises the cognate site, e.g., the template comprises an attR sequence if the genome comprises an attL sequence. In some embodiments, when attL/R recognition sites are used in a Gene Writing system, the system also comprises a recombination directionality factor (RDF) to enable recognition and recombination of these sites. In some embodiments, a Gene Writer polypeptide and a cognate RDF are provided as a fusion polypeptide. An exemplary recombinase-RDF fusion is described in Olorunniji et al. Nucleic Acids Res 45(14):8635-8645 (2017), which is incorporated herein by reference in its entirety.

In some embodiments, the protein component(s) of a Gene Writing™ system as described herein may be pre-associated with a template (e.g., a DNA template). For example, in some embodiments, the Gene Writer™ polypeptide may be first combined with the DNA template to form a deoxyribonucleoprotein (DNP) complex. In some embodiments, the DNP may be delivered to cells via, e.g., transfection, nucleofection, virus, vesicle, LNP, exosome, fusosome. In some embodiments, the template DNA may be first associated with a DNA-bending factor, e.g., HMGB1, in order to facilitate excision and transposition when subsequently contacted with the transposase component. Additional description of DNP delivery is found, for example, in Guha and Calos J Mol Biol (2020), which is herein incorporated by reference in its entirety.

In some embodiments, a polypeptide described herein comprises one or more (e.g., 2, 3, 4, 5) nuclear targeting sequences, for example a nuclear localization sequence (NLS). In some embodiments, the NLS is a bipartite NLS. In some embodiments, an NLS facilitates the import of a protein comprising an NLS into the cell nucleus. In some embodiments, the NLS is fused to the N-terminus of a Gene Writer described herein. In some embodiments, the NLS is fused to the C-terminus of the Gene Writer. In some embodiments, the NLS is fused to the N-terminus or the C-terminus of a Cas domain. In some embodiments, a linker sequence is disposed between the NLS and the neighboring domain of the Gene Writer.

In some embodiments, an NLS comprises the amino acid sequence MDSLLMNRRKFLYQFKNVRWAKGRRETYLC, PKKRKVEGADKRTADGSEFESPKKKRKV, RKSGKIAAIWKRPRKPKKKRKV KRTADGSEFESPKKKRKV, KKTELQTTNAENKTKKL, or KRGINDRNFWRGENGRKTR, KRPAATKKAGQAKKKK, or a functional fragment or variant thereof. Exemplary NLS sequences are also described in PCT/EP2000/011690, the contents of which are incorporated herein by reference for their disclosure of exemplary nuclear localization sequences.

In some embodiments, an NLS comprises an amino acid sequence as disclosed in Table 3. An NLS of this table may be utilized with one or more copies in a polypeptide in one or more locations in a polypeptide, e.g., 1, 2, 3 or more copies of an NLS in an N-terminal domain, between peptide domains, in a C-terminal domain, or in a combination of locations, in order to improve subcellular localization to the nucleus. Multiple unique sequences may be used within a single polypeptide. Sequences may be naturally monopartite or bipartite, e.g., having one or two stretches of basic amino acids, or may be used as chimeric bipartite sequences. Sequence references correspond to UniProt accession numbers, except where indicated as SeqNLS for sequences mined using a subcellular localization prediction algorithm (Lin et al BMC Bioinformat 13:157 (2012), incorporated herein by reference in its entirety).

TABLE 3
Exemplary nuclear localization signals
for use in Gene Writing systems

| Sequence | Sequence References |
|---|---|
| AHFKISGEKRPSTDPGKKAK | Q76IQ7 |
| NPKKKKKKDP | |
| AHRAKKMSKTHA | P21827 |
| ASPEYVNLPINGNG | SeqNLS |
| CTKRPRW | O88622, Q86W56, Q9QYM2, |
| | O02776 |
| DKAKRVSRNKSEKKRR | O15516, Q5RAK8, Q91YB2, |
| | Q91YB0, Q8QGQ6, O08785, |
| | Q9WVS9, Q6YGZ4 |
| EELRLKEELLKGIYA | Q9QY16, Q9UHL0, Q2TBP1, |
| | Q9QY15 |
| EEQLRRRKNSRLNNTG | G5EFF5 |
| EVLKVIRTGKRKKKAWKR | SeqNLS |
| MVTKVC | |
| HHHHHHHHHHHHQPH | Q63934, G3V7L5, Q12837 |
| HKKKHPDASVNFSEFSK | P10103, Q4R844, P12682, |
| | B0CM99, A9RA84, Q6YKA4, |
| | P09429, P63159, Q08IE6, |
| | P63158, Q9YH06, B1MTB0 |
| HKRTKK | Q2R2D5 |
| IINGRKLKLKKSRRRSSQTS | SeqNLS |
| NNSFTSRRS | |
| KAEQERRK | Q8LH59 |
| KEKRKRREELFIEQKKRK | SegNLS |
| KKGKDEWFSRGKKP | P30999 |
| KKGPSVQKRKKT | Q6ZN17 |
| KKKTVINDLLHYKKEK | SeqNLS, P32354 |
| KKNGGKGKNKPSAKIKK | SeqNLS |
| KKPKWDDFKKKKK | Q15397, Q8BKS9, Q562C7 |
| KKRKKD | SeqNLS, Q91Z62, Q1A730, |
| | Q969P5, Q2KHT6, Q9CPU7 |
| KKRRKRRRK | SeqNLS |
| KKRRRRARK | Q9UMS6, D4A702, Q91YE8 |
| KKSKRGR | Q9UBS0 |
| KKSRKRGS | B4FG96 |
| KKSTALSRELGKIMRRR | SeqNLS, P32354 |
| KKSYQDPEIIAHSRPRK | Q9U7C9 |
| KKTGKNRKLKSKRVKTR | Q9Z301, O54943, Q8K3T2 |
| KKVSIAGQSGKLWRWKR | Q6YUL8 |
| KKYENVVIKRSPRKRGRPR | SeqNLS |
| K | |
| KNKKRK | SeqNLS |
| KPKKKR | SegNLS |
| KRAMKDDSHGNSTSPKRRK | Q0E671 |
| KRANSNLVAAYEKAKKK | P23508 |
| KRASEDTTSGSPPKKSSAGP | Q9BZZ5, Q5R644 |
| KR | |
| KRFKRRWMVRKMKTKK | SeqNLS |
| KRGLNSSFETSPKKVK | Q8IV63 |
| KRGNSSIGPNDLSKRKQRK | SeqNLS |
| K | |
| KRIHSVSLSQSQIDPSKKVK | SegNLS |
| RAK | |
| KRKGKLKNKGSKRKK | O15381 |
| KRRRRRRREKRKR | Q96GM8 |
| KRSNDRTYSPEEEKQRRA | Q91ZF2 |
| KRTVATNGDASGAHRAKK | SeqNLS |
| MSK | |
| KRVYNKGEDEQEHLPKGKK | SeqNLS |
| R | |
| KSGKAPRRRAVSMDNSNK | Q9WVH4, O43524 |
| KVNFLDMSLDDIIIYKELE | Q9P127 |
| KVQHRIAKKTTRRRR | Q9DXE6 |
| LSPSLSPL | Q9Y261, P32182, P35583 |
| MDSLLMNRRKFLYQFKNVR | Q9GZX7 |
| WAKGRRETYLC | |
| MPQNEYIELHRKRYGYRLD | SeqNLS |
| YHEKKRKKESREAHERSKK | |
| AKKMIGLKAKLYHK | |
| MVQLRPRASR | SeqNLS |
| NNKLLAKRRKGGASPKDDP | Q965G5 |
| MDDIK | |
| NYKRPMDGTYGPPAKRHEG | O14497, A2BH40 |
| E | |
| PDTKRAKLDSSETTMVKKK | SeqNLS |
| PEKRTKI | SegNLS |
| PGGRGKKK | Q719N1, Q9UBP0, A2VDN5 |
| PGKMDKGEHRQERRDRPY | Q01844, Q61545 |
| PKKGDKYDKTD | Q45FA5 |
| PKKKSRK | O35914, Q01954 |
| PKKNKPE | Q22663 |
| PKKRAKV | P04295, P89438 |
| PKPKKLKVE | P55263, P55262, P55264, |
| | Q64640 |
| PKRGRGR | Q9FYS5, Q43386 |
| PKRRLVDDA | POC797 |
| PKRRRTY | SeqNLS |
| PLFKRR | A8X6H4, Q9TXJ0 |
| PLRKAKR | Q86WB0, Q5R8V9 |
| PPAKRKCIF | Q6AZ28, O75928, Q8C5D8 |
| PPARRRRL | Q8NAG6 |
| PPKKKRKV | Q3L6L5, P03070, P14999, |
| | P03071 |
| PPNKRMKVKH | Q8BN78 |
| PPRIYPQLPSAPT | P0C799 |
| PQRSPFPKSSVKR | SeqNLS |
| PRPRKVPR | P0C799 |
| PRRRVQRKR | SeqNLS, Q5R448, Q5TAQ9 |
| PRRVRLK | Q58DJ0, P56477, Q13568 |
| PSRKRPR | Q62315, Q5F363, Q92833 |
| PSSKKRKV | SeqNLS |
| PTKKRVK | P07664 |
| QRPGPYDRP | SeqNLS |
| RGKGGKGLGKGGAKRHRK | SeqNLS |
| RKAGKGGGGHKTTKKRSA | B4FG96 |
| KDEKVP | |
| RKIKLKRAK | A1L3G9 |
| RKIKRKRAK | B9X187 |
| RKKEAPGPREELRSRGR | O35126, P54258, Q5IS70, |
| | P54259 |
| RKKRKGK | SeqNLS, Q29243, Q62165, |
| | Q28685, O18738, Q9TSZ6, |
| | Q14118 |
| RKKRRQRRR | P04326, P69697, P69698, |
| | P05907, P20879, P04613, |
| | P19553, P0C1J9, P20893, |
| | P12506, P04612, Q73370, |
| | P0C1K0, P05906, P35965, |
| | P04609, P04610, P04614, |
| | P04608, P05905 |
| RKKSIPLSIKNLKRKHKRKK | Q9C0C9 |
| NKITR | |
| RKLVKPKNTKMKTKLRTNP | Q14190 |
| Y | |
| RKRLILSDKGQLDWKK | SeqNLS, Q91Z62, Q1A730, |
| | Q2KHT6, Q9CPU7 |
| RKRLKSK | Q13309 |
| RKRRVRDNM | Q8QPH4, Q809M7, A8C8X1, |
| | Q2VNC5, Q38SQ0, 089749, |
| | Q6DNQ9, Q809L9, Q0A429, |
| | Q20NV3, P16509, P16505, |
| | Q6DNQ5, P16506, Q6XT06, |
| | P26118, Q2ICQ2, Q2RCG8, |
| | Q0A2D0, QUA2H9, Q9IQ46, |
| | Q809M3, Q6J847, Q6J856, |
| | B4URE4, A4GCM7, Q0A440, |
| | P26120, P16511, |
| RKRSPKDKKEKDLDGAGKR | Q7RTP6 |
| RKT | |
| RKRTPRVDGQTGENDMNK | 094851 |
| RRRK | |
| RLPVRRRRRR | P04499, P12541, P03269, |
| | P48313, P03270 |
| RLRFRKPKSK | P69469 |
| RQQRKR | Q14980 |
| RRDLNSSFETSPKKVK | Q8K3G5 |
| RRDRAKLR | Q9SLB8 |
| RRGDGRRR | Q80WE1, Q5R9B4, Q06787, |
| | P35922 |
| RRGRKRKAEKQ | Q812D1, Q5XXA9, Q99JF8, |
| | Q8MJG1, Q66T72, 075475 |
| RRKKRR | QOVD86, Q58DS6, Q5R6G2, |
| | Q9ERI5, Q6AYK2, Q6NYC1 |
| RRKRSKSEDMDSVESKRRR | Q7TT18 |
| RRKRSR | Q99PU7, D3ZHS6, Q92560, |
| | A2VDM8 |
| RRPKGKTLQKRKPK | Q6ZN17 |
| RRRGFERFGPDNMGRKRK | Q63014, Q9DBR0 |
| RRRGKNKVAAQNCRK | SeqNLS |
| RRRKRR | Q5FVH8, Q6MZT1, Q08DH5, |
| | Q8BQP9 |
| RRRQKQKGGASRRR | SeqNLS |
| RRRREGPRARRRR | P08313, P10231 |
| RRTIRLKLVYDKCDRSCKIQ | SeqNLS |
| KKNRNKCQYCRFHKCLSVG | |
| MSHNAIRFGRMPRSEKAKL | |
| KAE | |
| RRVPQRKEVSRCRKCRK | Q5RJN4, Q32L09, Q8CAK3, |
| | Q9NUL5 |
| RVGGRRQAVECIEDLLNEP | P03255 |
| GQPLDLSCKRPRP | |
| RVVKLRIAP | P52639, Q8JMN0 |
| RVVRRR | P70278 |
| SKRKTKISRKTR | Q5RAY1, O00443 |
| SYVKTVPNRTRTYIKL | P21935 |
| TGKNEAKKRKIA | P52739, Q8K3J5, Q5RAU9 |
| TLSPASSPSSVSCPVIPASTD | SeqNLS |
| ESPGSALNI | |
| VSKKQRTGKKIH | P52739, Q8K3J5, Q5RAU9 |
| SPKKKRKVE | |
| KRTAD GSEFE SPKKKRKVE | |
| PAAKRVKLD | |
| PKKKRKV | |
| MDSLLMNRRKFLYQFKNVR | |
| WAKGRRETYLC | |
| SPKKKRKVEAS | |
| MAPKKKRKVGIHRGVP | |

In some embodiments, the NLS is a bipartite NLS. A bipartite NLS typically comprises two basic amino acid clusters separated by a spacer sequence (which may be, e.g., about 10 amino acids in length). A monopartite NLS typically lacks a spacer. An example of a bipartite NLS is the nucleoplasmin NLS, having the sequence KR[PAATKKAGQA]KKKK, wherein the spacer is bracketed. Another exemplary bipartite NLS has the sequence PKKKRKVEGADKRTADGSEFESPKKKRKV. Exemplary NLSs are described in International Application WO2020051561, which is herein incorporated by reference in its entirety, including for its disclosures regarding nuclear localization sequences.

### DNA Binding Domains

In some embodiments, a recombinase polypeptide (e.g., comprised in a system or cell as described herein), e.g., a tyrosine recombinase, comprises a DNA binding domain (e.g., a target binding domain or a template binding domain). In some embodiments, a recombinase polypeptide comprises the amino acid sequence of a DNA binding domain of a recombinase as described in Ioannidi et al. (2021, bioRxiv 2021.11.01.466786; doi: https://doi.org/10.1101/2021.11.01.466786; incorporated herein by reference in its entirety), or an amino acid sequence having at least 75%, 80%, 85%, 90%, 95%, 96%, 97%, 98%, or 99% sequence identity thereto. In some embodiments, a recombinase polypeptide comprises the amino acid sequence of a DNA binding domain of a recombinase as described in Anzalone et al. (2021, Nat. Biotechnol. doi: https://doi.org/10.1038/s41587-021-01133-w; incorporated herein by reference in its entirety), or an amino acid sequence having at least 75%, 80%, 85%, 90%, 95%, 96%, 97%, 98%, or 99% sequence identity thereto.

In some embodiments, a recombinase polypeptide described herein may be redirected to a defined target site in the human genome. In some embodiments, a recombinase described herein may be fused to a heterologous domain, e.g., a heterologous DNA binding domain. In some embodiments, a recombinase may be fused to a heterologous DNA binding domain, e.g., a DNA binding domain from a zinc finger, TAL, meganuclease, transcription factor, or sequence-guided DNA binding element. In some embodiments, a recombinase may be fused to a DNA binding domain from a sequence-guided DNA binding element, e.g., a CRISPR-associated (Cas) DNA binding element, e.g., a Cas9. In some embodiments, a DNA binding element fused to a recombinase domain may contain mutations inactivating other catalytic functions, e.g., mutations inactivating endonuclease activity, e.g., mutations creating an inactivated meganuclease or partially or completely inactivate Cas protein, e.g., mutations creating a nickase Cas9 or dead Cas9 (dCas9). As an example, Standage-Beier et al. CRISPR J2(4):209-222 (2019), describes the use of a dCas9 fused to the Tn3 resolvase (integrase Cas9, iCas9) that employs appropriate spacing of two monomeric fusion proteins at the target site for cooperative targeting for the sequence-specific integration of reporter systems into the genome of HEK293 cells. Additional examples of recombinase targeting by DNA binding domains include zinc finger fusions (zinc-finger recombinases, ZFRs (Gaj et al. Nucleic Acids Res 41(6):3937-3946 (2013)); RecZFs (Gersbach et al. Nucleic Acids Res 38(12):4198-4206 (2010))), TALE fusions (TALE recombinases, TALERs (Mercer et al. Nucleic Acids Res 40(21):11163-11172 (2012))), and dCas9 fusions (recombinase Cas9, recCas9 (Chaikind et al. Nucleic Acids Res 44(20):9758-9770 (2016)); integrase Cas9, iCas9 (Standage-Beier et al. CRISPR J 2(4):209-222 (2019))), all of which are incorporated herein by reference.

In some embodiments, a DNA binding domain comprises a Streptococcus pyogenes Cas9 (SpCas9) or a functional fragment or variant thereof. In some embodiments, the DNA binding domain comprises a modified SpCas9. In embodiments, the modified SpCas9 comprises a modification that alters protospacer-adjacent motif (PAM) specificity. In embodiments, the PAM has specificity for the nucleic acid sequence 5′-NGT-3′. In embodiments, the modified SpCas9 comprises one or more amino acid substitutions, e.g., at one or more of positions L1111, D1135, G1218, E1219, A1322, of R1335, e.g., selected from L1111R, D1135V, G1218R, E1219F, A1322R, R1335V. In embodiments, the modified SpCas9 comprises the amino acid substitution T1337R and one or more additional amino acid substitutions, e.g., selected from L1111, D1135L, S1136R, G1218S, E1219V, D1332A, D1332S, D1332T, D1332V, D1332L, D1332K, D1332R, R1335Q, T1337, T1337L, T1337Q, T13371, T1337V, T1337F, T1337S, T1337N, T1337K, T1337H, T1337Q, and T1337M, or corresponding amino acid substitutions thereto. In embodiments, the modified SpCas9 comprises: (i) one or more amino acid substitutions selected from D1135L, S1136R, G1218S, E1219V, A1322R, R1335Q, and T1337; and (ii) one or more amino acid substitutions selected from L1111R, G1218R, E1219F, D1332A, D1332S, D1332T, D1332V, D1332L, D1332K, D1332R, T1337L, T1337I, T1337V, T1337F, T1337S, T1337N, T1337K, T1337R, T1337H, T1337Q, and T1337M, or corresponding amino acid substitutions thereto.

In some embodiments, a Gene Writer may comprise a Cas protein as listed in Table 4. The predicted or validated nickase mutations for installing Nickase activity in the Cas protein as shown in Table 4, are based on the signature of the SpCas9(N863A) mutation. In some embodiments, system described herein comprises a GeneWriter protein described herein and a Cas protein of Table 4.

TABLE 4
CRISPR/Cas Proteins, Species, and Mutations

| | Parental | | Nickase |
|---|---|---|---|
| Variant | Host | Protein Sequence | Mutation |
| Nme2Cas9 | Neisseria | MAAFKPNPINYILGLDIGIASVGWAMVEIDEEENPIRLIDLGVRVFERAEVP | N611A |
| | meningitidis | KTGDSLAMARRLARSVRRLTRRRAHRLLRARRLLKREGVLQAADFDENG | |
| | | LIKSLPNTPWQLRAAALDRKLTPLEWSAVLLHLIKHRGYLSQRKNEGETAD | |
| | | KELGALLKGVANNAHALQTGDFRTPAELALNKFEKESGHIRNQRGDYSHT | |
| | | FSRKDLQAELILLFEKQKEFGNPHVSGGLKEGIETLLMTQRPALSGDAVQK | |
| | | MLGHCTFEPAEPKAAKNTYTAERFIWLTKLNNLRILEQGSERPLTDTERAT | |
| | | LMDEPYRKSKLTYAQARKLLGLEDTAFFKGLRYGKDNAEASTLMEMKAY | |
| | | HAISRALEKEGLKDKKSPLNLSSELQDEIGTAFSLFKTDEDITGRLKDRVQP | |
| | | EILEALLKHISFDKFVQISLKALRRIVPLMEQGKRYDEACAEIYGDHYGKKN | |
| | | TEEKIYLPPIPADEIRNPVVLRALSQARKVINGVVRRYGSPARIHIETAREVG | |
| | | KSFKDRKEIEKRQEENRKDREKAAAKFREYFPNFVGEPKSKDILKLRLYEQ | |
| | | QHGKCLYSGKEINLVRLNEKGYVEIDHALPFSRTWDDSFNNKVLVLGSEN | |
| | | QNKGNQTPYEYFNGKDNSREWQEFKARVETSRFPRSKKQRILLQKFDEDG | |
| | | FKECNLNDTRYVNRFLCQFVADHILLTGKGKRRVFASNGQITNLLRGFWG | |
| | | LRKVRAENDRHHALDAVVVACSTVAMQQKITRFVRYKEMNAFDGKTIDK | |
| | | ETGKVLHQKTHFPQPWEFFAQEVMIRVFGKPDGKPEFEEADTPEKLRTLLA | |
| | | EKLSSRPEAVHEYVTPLFVSRAPNRKMSGAHKDTLRSAKRFVKHNEKISVK | |
| | | RVWLTEIKLADLENMVNYKNGREIELYEALKARLEAYGGNAKQAFDPKD | |
| | | NPFYKKGGQLVKAVRVEKTQESGVLLNKKNAYTIADNGDMVRVDVFCK | |
| | | VDKKGKNQYFIVPIYAWQVAENILPDIDCKGYRIDDSYTFCFSLHKYDLIAF | |
| | | QKDEKSKVEFAYYINCDSSNGRFYLAWHDKGSKEQQFRISTQNLVLIQKY | |
| | | QVNELGKEIRPCRLKKRPPVR | |
| PpnCas9 | Pasteurella | MQNNPLNYILGLDLGIASIGWAVVEIDEESSPIRLIDVGVRTFERAEVAKTG | N605A |
| | pneumotropica | ESLALSRRLARSSRRLIKRRAERLKKAKRLLKAEKILHSIDEKLPINVWQLR | |
| | | VKGLKEKLERQEWAAVLLHLSKHRGYLSQRKNEGKSDNKELGALLSGIAS | |
| | | NHQMLQSSEYRTPAEIAVKKFQVEEGHIRNQRGSYTHTFSRLDLLAEMELL | |
| | | FQRQAELGNSYTSTTLLENLTALLMWQKPALAGDAILKMLGKCTFEPSEY | |
| | | KAAKNSYSAERFVWLTKLNNLRILENGTERALNDNERFALLEQPYEKSKL | |
| | | TYAQVRAMLALSDNAIFKGVRYLGEDKKTVESKTTLIEMKFYHQIRKTLG | |
| | | SAELKKEWNELKGNSDLLDEIGTAFSLYKTDDDICRYLEGKLPERVLNALL | |
| | | ENLNFDKFIQLSLKALHQILPLMLQGQRYDEAVSAIYGDHYGKKSTETTRL | |
| | | LPTIPADEIRNPVVLRTLTQARKVINAVVRLYGSPARIHIETAREVGKSYQD | |
| | | RKKLEKQQEDNRKQRESAVKKFKEMFPHFVGEPKGKDILKMRLYELQQA | |
| | | KCLYSGKSLELHRLLEKGYVEVDHALPFSRTWDDSFNNKVLVLANENQN | |
| | | KGNLTPYEWLDGKNNSERWQHFVVRVQTSGFSYAKKQRILNHKLDEKGFI | |
| | | ERNLNDTRYVARFLCNFIADNMLLVGKGKRNVFASNGQITALLRHRWGL | |
| | | QKVREQNDRHHALDAVVVACSTVAMQQKITRFVRYNEGNVFSGERIDRE | |
| | | TGEIIPLHFPSPWAFFKENVEIRIFSENPKLELENRLPDYPQYNHEWVQPLFV | |
| | | SRMPTRKMTGQGHMETVKSAKRLNEGLSVLKVPLTQLKLSDLERMVNRD | |
| | | REIALYESLKARLEQFGNDPAKAFAEPFYKKGGALVKAVRLEQTQKSGVL | |
| | | VRDGNGVADNASMVRVDVFTKGGKYFLVPIYTWQVAKGILPNRAATQGK | |
| | | DENDWDIMDEMATFQFSLCQNDLIKLVTKKKTIFGYFNGLNRATSNINIKE | |
| | | HDLDKSKGKLGIYLEVGVKLAISLEKYQVDELGKNIRPCRPTKRQHVR | |
| SauCas9 | Staphylococcus | MKRNYILGLDIGITSVGYGIIDYETRDVIDAGVRLFKEANVENNEGRRSKR | N580A |
| | aureus | GARRLKRRRRHRIQRVKKLLFDYNLLTDHSELSGINPYEARVKGLSQKLSE | |
| | | EEFSAALLHLAKRRGVHNVNEVEEDTGNELSTKEQISRNSKALEEKYVAEL | |
| | | QLERLKKDGEVRGSINRFKTSDYVKEAKQLLKVQKAYHQLDQSFIDTYIDL | |
| | | LETRRTYYEGPGEGSPFGWKDIKEWYEMLMGHCTYFPEELRSVKYAYNA | |
| | | DLYNALNDLNNLVITRDENEKLEYYEKFQIIENVFKQKKKPTLKQIAKEILV | |
| | | NEEDIKGYRVTSTGKPEFTNLKVYHDIKDITARKEIIENAELLDQIAKILTIY | |
| | | QSSEDIQEELTNLNSELTQEEIEQISNLKGYTGTHNLSLKAINLILDELWHTN | |
| | | DNQIAIFNRLKLVPKKVDLSQQKEIPTTLVDDFILSPVVKRSFIQSIKVINAII | |
| | | KKYGLPNDIIIELAREKNSKDAQKMINEMQKRNRQTNERIEEIIRTTGKENA | |
| | | KYLIEKIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRISKTK | |
| | | KEYLLEERDINRFSVQKDFINRNLVDTRYATRGLMNLLRSYFRVNNLDVK | |
| | | VKSINGGFTSFLRRKWKFKKERNKGYKHHAEDALIIANADFIFKEWKKLD | |
| | | KAKKVMENQMFEEKQAESMPEIETEQEYKEIFITPHQIKHIKDFKDYKYSH | |
| | | RVDKKPNRELINDTLYSTRKDDKGNTLIVNNLNGLYDKDNDKLKKLINKS | |
| | | PEKLLMYHHDPQTYQKLKLIMEQYGDEKNPLYKYYEETGNYLTKYSKKD | |
| | | NGPVIKKIKYYGNKLNAHLDITDDYPNSRNKVVKLSLKPYRFDVYLDNGV | |
| | | YKFVTVKNLDVIKKENYYEVNSKCYEEAKKLKKISNQAEFIASFYNNDLIK | |
| | | INGELYRVIGVNNDLLNRIEVNMIDITYREYLENMNDKRPPRIIKTIASKTQS | |
| | | IKKYSTDILGNLYEVKSKKHPQIIKKG | |
| SauCas9- | Staphylococcus | MKRNYILGLDIGITSVGYGIIDYETRDVIDAGVRLFKEANVENNEGRRSKR | N580A |
| KKH | aureus | GARRLKRRRRHRIQRVKKLLFDYNLLTDHSELSGINPYEARVKGLSQKLSE | |
| | | EEFSAALLHLAKRRGVHNVNEVEEDTGNELSTKEQISRNSKALEEKYVAEL | |
| | | QLERLKKDGEVRGSINRFKTSDYVKEAKQLLKVQKAYHQLDQSFIDTYIDL | |
| | | LETRRTYYEGPGEGSPFGWKDIKEWYEMLMGHCTYFPEELRSVKYAYNA | |
| | | DLYNALNDLNNLVITRDENEKLEYYEKFQIIENVFKQKKKPTLKQIAKEILV | |
| | | NEEDIKGYRVTSTGKPEFTNLKVYHDIKDITARKEIIENAELLDQIAKILTIY | |
| | | QSSEDIQEELTNLNSELTQEEIEQISNLKGYTGTHNLSLKAINLILDELWHTN | |
| | | DNQIAIFNRLKLVPKKVDLSQQKEIPTTLVDDFILSPVVKRSFIQSIKVINAII | |
| | | KKYGLPNDIIIELAREKNSKDAQKMINEMQKRNRQTNERIEEIIRTTGKENA | |
| | | KYLIEKIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRISKTK | |
| | | KEYLLEERDINRFSVQKDFINRNLVDTRYATRGLMNLLRSYFRVNNLDVK | |
| | | VKSINGGFTSFLRRKWKFKKERNKGYKHHAEDALIIANADFIFKEWKKLD | |
| | | KAKKVMENQMFEEKQAESMPEIETEQEYKEIFITPHQIKHIKDFKDYKYSH | |
| | | RVDKKPNRKLINDTLYSTRKDDKGNTLIVNNLNGLYDKDNDKLKKLINKS | |
| | | PEKLLMYHHDPQTYQKLKLIMEQYGDEKNPLYKYYEETGNYLTKYSKKD | |
| | | NGPVIKKIKYYGNKLNAHLDITDDYPNSRNKVVKLSLKPYRFDVYLDNGV | |
| | | YKFVTVKNLDVIKKENYYEVNSKCYEEAKKLKKISNQAEFIASFYKNDLIK | |
| | | INGELYRVIGVNNDLLNRIEVNMIDITYREYLENMNDKRPPHIIKTIASKTQS | |
| | | IKKYSTDILGNLYEVKSKKHPQIIKKG | |
| SauriCas9 | Staphylococcus | MQENQQKQNYILGLDIGITSVGYGLIDSKTREVIDAGVRLFPEADSENNSNR | N588A |
| | auricularis | RSKRGARRLKRRRIHRLNRVKDLLADYQMIDLNNVPKSTDPYTIRVKGLR | |
| | | EPLTKEEFAIALLHIAKRRGLHNISVSMGDEEQDNELSTKQQLQKNAQQLQ | |
| | | DKYVCELQLERLTNINKVRGEKNRFKTEDFVKEVKQLCETQRQYHNIDDQ | |
| | | FIQQYIDLVSTRREYFEGPGNGSPYGWDGDLLKWYEKLMGRCTYFPEELR | |
| | | SVKYAYSADLFNALNDLNNLVVTRDDNPKLEYYEKYHIIENVFKQKKNPT | |
| | | LKQIAKEIGVQDYDIRGYRITKSGKPQFTSFKLYHDLKNIFEQAKYLEDVE | |
| | | MLDEIAKILTIYQDEISIKKALDQLPELLTESEKSQIAQLTGYTGTHRLSLKCI | |
| | | HIVIDELWESPENQMEIFTRLNLKPKKVEMSEIDSIPTTLVDEFILSPVVKRA | |
| | | FIQSIKVINAVINRFGLPEDIIIELAREKNSKDRRKFINKLQKQNEATRKKIEQ | |
| | | LLAKYGNTNAKYMIEKIKLHDMQEGKCLYSLEAIPLEDLLSNPTHYEVDHI | |
| | | IPRSVSFDNSLNNKVLVKQSENSKKGNRTPYQYLSSNESKISYNQFKQHILN | |
| | | LSKAKDRISKKKRDMLLEERDINKFEVQKEFINRNLVDTRYATRELSNLLK | |
| | | TYFSTHDYAVKVKTINGGFTNHLRKVWDFKKHRNHGYKHHAEDALVIAN | |
| | | ADFLFKTHKALRRTDKILEQPGLEVNDTTVKVDTEEKYQELFETPKQVKNI | |
| | | KQFRDFKYSHRVDKKPNRQLINDTLYSTREIDGETYVVQTLKDLYAKDNE | |
| | | KVKKLFTERPQKILMYQHDPKTFEKLMTILNQYAEAKNPLAAYYEDKGEY | |
| | | VTKYAKKGNGPAIHKIKYIDKKLGSYLDVSNKYPETQNKLVKLSLKSFRFD | |
| | | IYKCEQGYKMVSIGYLDVLKKDNYYYIPKDKYEAEKQKKKIKESDLFVGS | |
| | | FYYNDLIMYEDELFRVIGVNSDINNLVELNMVDITYKDFCEVNNVTGEKRI | |
| | | KKTIGKRVVLIEKYTTDILGNLYKTPLPKKPQLIFKRGEL | |
| SauriCas9- | Staphylococcus | MQENQQKQNYILGLDIGITSVGYGLIDSKTREVIDAGVRLFPEADSENNSNR | N588A |
| KKH | auricularis | RSKRGARRLKRRRIHRLNRVKDLLADYQMIDLNNVPKSTDPYTIRVKGLR | |
| | | EPLTKEEFAIALLHIAKRRGLHNISVSMGDEEQDNELSTKQQLQKNAQQLQ | |
| | | DKYVCELQLERLTNINKVRGEKNRFKTEDFVKEVKQLCETQRQYHNIDDQ | |
| | | FIQQYIDLVSTRREYFEGPGNGSPYGWDGDLLKWYEKLMGRCTYFPEELR | |
| | | SVKYAYSADLFNALNDLNNLVVTRDDNPKLEYYEKYHIIENVFKQKKNPT | |
| | | LKQIAKEIGVQDYDIRGYRITKSGKPQFTSFKLYHDLKNIFEQAKYLEDVE | |
| | | MLDEIAKILTIYQDEISIKKALDQLPELLTESEKSQIAQLTGYTGTHRLSLKCI | |
| | | HIVIDELWESPENQMEIFTRLNLKPKKVEMSEIDSIPTTLVDEFILSPVVKRA | |
| | | FIQSIKVINAVINRFGLPEDIIIELAREKNSKDRRKFINKLQKQNEATRKKIEQ | |
| | | LLAKYGNTNAKYMIEKIKLHDMQEGKCLYSLEAIPLEDLLSNPTHYEVDHI | |
| | | IPRSVSFDNSLNNKVLVKQSENSKKGNRTPYQYLSSNESKISYNQFKQHILN | |
| | | LSKAKDRISKKKRDMLLEERDINKFEVQKEFINRNLVDTRYATRELSNLLK | |
| | | TYFSTHDYAVKVKTINGGFTNHLRKVWDFKKHRNHGYKHHAEDALVIAN | |
| | | ADFLFKTHKALRRTDKILEQPGLEVNDTTVKVDTEEKYQELFETPKQVKNI | |
| | | KQFRDFKYSHRVDKKPNRKLINDTLYSTREIDGETYVVQTLKDLYAKDNE | |
| | | KVKKLFTERPQKILMYQHDPKTFEKLMTILNQYAEAKNPLAAYYEDKGEY | |
| | | VTKYAKKGNGPAIHKIKYIDKKLGSYLDVSNKYPETQNKLVKLSLKSFRFD | |
| | | IYKCEQGYKMVSIGYLDVLKKDNYYYIPKDKYEAEKQKKKIKESDLFVGS | |
| | | FYKNDLIMYEDELFRVIGVNSDINNLVELNMVDITYKDFCEVNNVTGEKHI | |
| | | KKTIGKRVVLIEKYTTDILGNLYKTPLPKKPQLIFKRGEL | |
| ScaCas9- | Streptococcus | MEKKYSIGLDIGTNSVGWAVITDDYKVPSKKFKVLGNTNRKSIKKNLMGA | N872A |
| Sc++ | canis | LLFDSGETAEATRLKRTARRRYTRRKNRIRYLQEIFANEMAKLDDSFFQRL | |
| | | EESFLVEEDKKNERHPIFGNLADEVAYHRNYPTIYHLRKKLADSPEKADLR | |
| | | LIYLALAHIIKFRGHFLIEGKLNAENSDVAKLFYQLIQTYNQLFEESPLDEIE | |
| | | VDAKGILSARLSKSKRLEKLIAVFPNEKKNGLFGNIIALALGLTPNFKSNFD | |
| | | LTEDAKLQLSKDTYDDDLDELLGQIGDQYADLFSAAKNLSDAILLSDILRS | |
| | | NSEVTKAPLSASMVKRYDEHHQDLALLKTLVRQQFPEKYAEIFKDDTKNG | |
| | | YAGYVGADKKLRKRSGKLATEEEFYKFIKPILEKMDGAEELLAKLNRDDL | |
| | | LRKQRTFDNGSIPHQIHLKELHAILRRQEEFYPFLKENREKIEKILTFRIPYYV | |
| | | GPLARGNSRFAWLTRKSEEAITPWNFEEVVDKGASAQSFIERMTNFDEQLP | |
| | | NKKVLPKHSLLYEYFTVYNELTKVKYVTERMRKPEFLSGEQKKAIVDLLF | |
| | | KTNRKVTVKQLKEDYFKKIECFDSVEIIGVEDRFNASLGTYHDLLKIIKDKD | |
| | | FLDNEENEDILEDIVLTLTLFEDREMIEERLKTYAHLFDDKVMKQLKRRHY | |
| | | TGWGRLSRKMINGIRDKQSGKTILDFLKSDGFSNRNFMQLIHDDSLTFKEEI | |
| | | EKAQVSGQGDSLHEQIADLAGSPAIKKGILQTVKIVDELVKVMGHKPENIV | |
| | | IEMARENQTTTKGLQQSRERKKRIEEGIKELESQILKENPVENTQLQNEKLY | |
| | | LYYLQNGRDMYVDQELDINRLSDYDVDHIVPQSFIKDDSIDNKVLTRSVEN | |
| | | RGKSDNVPSEEVVKKMKNYWRQLLNAKLITQRKFDNLTKAERGGLSEAD | |
| | | KAGFIKRQLVETRQITKHVARILDSRMNTKRDKNDKPIREVKVITLKSKLVS | |
| | | DFRKDFQLYKVRDINNYHHAHDAYLNAVVGTALIKKYPKLESEFVYGDY | |
| | | KVYDVRKMIAKSEQEIGKATAKRFFYSNIMNFFKTEVKLANGEIRKRPLIET | |
| | | NGETGEVVWNKEKDFATVRKVLAMPQVNIVKKTEVQTGGFSKESILSKRE | |
| | | SAKLIPRKKGWDTRKYGGFGSPTVAYSILVVAKVEKGKAKKLKSVKVLVG | |
| | | ITIMEKGSYEKDPIGFLEAKGYKDIKKELIFKLPKYSLFELENGRRRMLASA | |
| | | KELQKANELVLPQHLVRLLYYTQNISATTGSNNLGYIEQHREEFKEIFEKIID | |
| | | FSEKYILKNKVNSNLKSSFDEQFAVSDSILLSNSFVSLLKYTSFGASGGFTFL | |
| | | DLDVKQGRLRYQTVTEVLDATLIYQSITGLYETRTDLSQLGGD | |
| SpyCas9 | Streptococcus | MDKKYSIGLDIGTNSVGWAVITDEYKVPSKKFKVLGNTDRHSIKKNLIGAL | N863A |
| | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFDSPTVAYSVLVVAKVEKGKSKKLKSVKELLGITIMERSS | |
| | | FEKNPIDFLEAKGYKEVKKDLIIKLPKYSLFELENGRKRMLASAGELQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTIDRKRYTSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| NG | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESIRPKRNSDKLIARK | |
| | | KDWDPKKYGGFVSPTVAYSVLVVAKVEKGKSKKLKSVKELLGITIMERSS | |
| | | FEKNPIDFLEAKGYKEVKKDLIIKLPKYSLFELENGRKRMLASARFLQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPRAFKYFD | |
| | | TTIDRKVYRSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| SpRY | pyogenes | LFDSGETAERTRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESIRPKRNSDKLIARK | |
| | | KDWDPKKYGGFLWPTVAYSVLVVAKVEKGKSKKLKSVKELLGITIMERSS | |
| | | FEKNPIDFLEAKGYKEVKKDLIIKLPKYSLFELENGRKRMLASAKQLQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTRLGAPRAFKYFD | |
| | | TTIDPKQYRSTKEVLDATLIHQSITGLYETRIDLSQLGGD | |
| St1Cas9 | Streptococcus | MSDLVLGLDIGIGSVGVGILNKVTGEIIHKNSRIFPAAQAENNLVRRTNRQG | N622A |
| | thermophilus | RRLARRKKHRRVRLNRLFEESGLITDFTKISINLNPYQLRVKGLTDELSNEE | |
| | | LFIALKNMVKHRGISYLDDASDDGNSSVGDYAQIVKENSKQLETKTPGQIQ | |
| | | LERYQTYGQLRGDFTVEKDGKKHRLINVFPTSAYRSEALRILQTQQEFNPQI | |
| | | TDEFINRYLEILTGKRKYYHGPGNEKSRTDYGRYRTSGETLDNIFGILIGKC | |
| | | TFYPDEFRAAKASYTAQEFNLLNDLNNLTVPTETKKLSKEQKNQIINYVKN | |
| | | EKAMGPAKLFKYIAKLLSCDVADIKGYRIDKSGKAEIHTFEAYRKMKTLET | |
| | | LDIEQMDRETLDKLAYVLTLNTEREGIQEALEHEFADGSFSQKQVDELVQF | |
| | | RKANSSIFGKGWHNFSVKLMMELIPELYETSEEQMTILTRLGKQKTTSSSN | |
| | | KTKYIDEKLLTEEIYNPVVAKSVRQAIKIVNAAIKEYGDFDNIVIEMARETN | |
| | | EDDEKKAIQKIQKANKDEKDAAMLKAANQYNGKAELPHSVFHGHKQLAT | |
| | | KIRLWHQQGERCLYTGKTISIHDLINNSNQFEVDHILPLSITFDDSLANKVLV | |
| | | YATANQEKGQRTPYQALDSMDDAWSFRELKAFVRESKTLSNKKKEYLLT | |
| | | EEDISKFDVRKKFIERNLVDTRYASRVVLNALQEHFRAHKIDTKVSVVRGQ | |
| | | FTSQLRRHWGIEKTRDTYHHHAVDALIIAASSQLNLWKKQKNTLVSYSED | |
| | | QLLDIETGELISDDEYKESVFKAPYQHFVDTLKSKEFEDSILFSYQVDSKFN | |
| | | RKISDATIYATRQAKVGKDKADETYVLGKIKDIYTQDGYDAFMKIYKKDK | |
| | | SKFLMYRHDPQTFEKVIEPILENYPNKQINEKGKEVPCNPFLKYKEEHGYIR | |
| | | KYSKKGNGPEIKSLKYYDSKLGNHIDITPKDSNNKVVLQSVSPWRADVYF | |
| | | NKTTGKYEILGLKYADLQFEKGTGTYKISQEKYNDIKKKEGVDSDSEFKFT | |
| | | LYKNDLLLVKDTETKEQQLFRFLSRTMPKQKHYVELKPYDKQKFEGGEAL | |
| | | IKVLGNVANSGQCKKGLGKSNISIYKVRTDVLGNQHIIKNEGDKPKLDF | |
| BlatCas9 | Brevibacillus | MAYTMGIDVGIASCGWAIVDLERQRIIDIGVRTFEKAENPKNGEALAVPRR | N607A |
| | laterosporus | EARSSRRRLRRKKHRIERLKHMFVRNGLAVDIQHLEQTLRSQNEIDVWQL | |
| | | RVDGLDRMLTQKEWLRVLIHLAQRRGFQSNRKTDGSSEDGQVLVNVTEN | |
| | | DRLMEEKDYRTVAEMMVKDEKFSDHKRNKNGNYHGVVSRSSLLVEIHTL | |
| | | FETQRQHHNSLASKDFELEYVNIWSAQRPVATKDQIEKMIGTCTFLPKEKR | |
| | | APKASWHFQYFMLLQTINHIRITNVQGTRSLNKEEIEQVVNMALTKSKVSY | |
| | | HDTRKILDLSEEYQFVGLDYGKEDEKKKVESKETIIKLDDYHKLNKIFNEV | |
| | | ELAKGETWEADDYDTVAYALTFFKDDEDIRDYLQNKYKDSKNRLVKNLA | |
| | | NKEYTNELIGKVSTLSFRKVGHLSLKALRKIIPFLEQGMTYDKACQAAGFD | |
| | | FQGISKKKRSVVLPVIDQISNPVVNRALTQTRKVINALIKKYGSPETIHIETA | |
| | | RELSKTFDERKNITKDYKENRDKNEHAKKHLSELGIINPTGLDIVKYKLWC | |
| | | EQQGRCMYSNQPISFERLKESGYTEVDHIIPYSRSMNDSYNNRVLVMTREN | |
| | | REKGNQTPFEYMGNDTQRWYEFEQRVTTNPQIKKEKRQNLLLKGFTNRRE | |
| | | LEMLERNLNDTRYITKYLSHFISTNLEFSPSDKKKKVVNTSGRITSHLRSRW | |
| | | GLEKNRGQNDLHHAMDAIVIAVTSDSFIQQVTNYYKRKERRELNGDDKFP | |
| | | LPWKFFREEVIARLSPNPKEQIEALPNHFYSEDELADLQPIFVSRMPKRSITG | |
| | | EAHQAQFRRVVGKTKEGKNITAKKTALVDISYDKNGDFNMYGRETDPAT | |
| | | YEAIKERYLEFGGNVKKAFSTDLHKPKKDGTKGPLIKSVRIMENKTLVHPV | |
| | | NKGKGVVYNSSIVRTDVFQRKEKYYLLPVYVTDVTKGKLPNKVIVAKKG | |
| | | YHDWIEVDDSFTFLFSLYPNDLIFIRQNPKKKISLKKRIESHSISDSKEVQEIH | |
| | | AYYKGVDSSTAAIEFIIHDGSYYAKGVGVQNLDCFEKYQVDILGNYFKVK | |
| | | GEKRLELETSDSNHKGKDVNSIKSTSR | |
| cCas9-v16 | Staphylococcus | MKRNYILGLDIGITSVGYGIIDYETRDVIDAGVRLFKEANVENNEGRRSKR | N580A |
| | aureus | GARRLKRRRRHRIQRVKKLLFDYNLLTDHSELSGINPYEARVKGLSQKLSE | |
| | | EEFSAALLHLAKRRGVHNVNEVEEDTGNELSTKEQISRNSKALEEKYVAEL | |
| | | QLERLKKDGEVRGSINRFKTSDYVKEAKQLLKVQKAYHQLDQSFIDTYIDL | |
| | | LETRRTYYEGPGEGSPFGWKDIKEWYEMLMGHCTYFPEELRSVKYAYNA | |
| | | DLYNALNDLNNLVITRDENEKLEYYEKFQIIENVFKQKKKPTLKQIAKEILV | |
| | | NEEDIKGYRVTSTGKPEFTNLKVYHDIKDITARKEIIENAELLDQIAKILTIY | |
| | | QSSEDIQEELTNLNSELTQEEIEQISNLKGYTGTHNLSLKAINLILDELWHTN | |
| | | DNQIAIFNRLKLVPKKVDLSQQKEIPTTLVDDFILSPVVKRSFIQSIKVINAII | |
| | | KKYGLPNDIIIELAREKNSKDAQKMINEMQKRNRQTNERIEEIIRTTGKENA | |
| | | KYLIEKIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRISKTK | |
| | | KEYLLEERDINRFSVQKDFINRNLVDTRYATRGLMNLLRSYFRVNNLDVK | |
| | | VKSINGGFTSFLRRKWKFKKERNKGYKHHAEDALIIANADFIFKEWKKLD | |
| | | KAKKVMENQMFEEKQAESMPEIETEQEYKEIFITPHQIKHIKDFKDYKYSH | |
| | | RVDKKPNRKLINDTLYSTRKDDKGNTLIVNNLNGLYDKDNDKLKKLINKS | |
| | | PEKLLMYHHDPQTYQKLKLIMEQYGDEKNPLYKYYEETGNYLTKYSKKD | |
| | | NGPVIKKIKYYGNKLNAHLDITDDYPNSRNKVVKLSLKPYRFDVYLDNGV | |
| | | YKFVTVKNLDVIKKENYYEVNSKCYEEAKKLKKISNQAEFIASFYKNDLIK | |
| | | INGELYRVIGVNSDKNNLIEVNMIDITYREYLENMNDKRPPHIIKTIASKTQS | |
| | | IKKYSTDILGNLYEVKSKKHPQIIKKG | |
| cCas9-v17 | Staphylococcus | MKRNYILGLDIGITSVGYGIIDYETRDVIDAGVRLFKEANVENNEGRRSKR | N580A |
| | aureus | GARRLKRRRRHRIQRVKKLLFDYNLLTDHSELSGINPYEARVKGLSQKLSE | |
| | | EEFSAALLHLAKRRGVHNVNEVEEDTGNELSTKEQISRNSKALEEKYVAEL | |
| | | QLERLKKDGEVRGSINRFKTSDYVKEAKQLLKVQKAYHQLDQSFIDTYIDL | |
| | | LETRRTYYEGPGEGSPFGWKDIKEWYEMLMGHCTYFPEELRSVKYAYNA | |
| | | DLYNALNDLNNLVITRDENEKLEYYEKFQIIENVFKQKKKPTLKQIAKEILV | |
| | | NEEDIKGYRVTSTGKPEFTNLKVYHDIKDITARKEIIENAELLDQIAKILTIY | |
| | | QSSEDIQEELTNLNSELTQEEIEQISNLKGYTGTHNLSLKAINLILDELWHTN | |
| | | DNQIAIFNRLKLVPKKVDLSQQKEIPTTLVDDFILSPVVKRSFIQSIKVINAII | |
| | | KKYGLPNDIIIELAREKNSKDAQKMINEMQKRNRQTNERIEEIIRTTGKENA | |
| | | KYLIEKIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRISKTK | |
| | | KEYLLEERDINRFSVQKDFINRNLVDTRYATRGLMNLLRSYFRVNNLDVK | |
| | | VKSINGGFTSFLRRKWKFKKERNKGYKHHAEDALIIANADFIFKEWKKLD | |
| | | KAKKVMENQMFEEKQAESMPEIETEQEYKEIFITPHQIKHIKDFKDYKYSH | |
| | | RVDKKPNRKLINDTLYSTRKDDKGNTLIVNNLNGLYDKDNDKLKKLINKS | |
| | | PEKLLMYHHDPQTYQKLKLIMEQYGDEKNPLYKYYEETGNYLTKYSKKD | |
| | | NGPVIKKIKYYGNKLNAHLDITDDYPNSRNKVVKLSLKPYRFDVYLDNGV | |
| | | YKFVTVKNLDVIKKENYYEVNSKCYEEAKKLKKISNQAEFIASFYKNDLIK | |
| | | INGELYRVIGVNNSTRNIVELNMIDITYREYLENMNDKRPPHIIKTIASKTQS | |
| | | IKKYSTDILGNLYEVKSKKHPQIIKKG | |
| cCas9-v21 | Staphylococcus | MKRNYILGLDIGITSVGYGIIDYETRDVIDAGVRLFKEANVENNEGRRSKR | N580A |
| | aureus | GARRLKRRRRHRIQRVKKLLFDYNLLTDHSELSGINPYEARVKGLSQKLSE | |
| | | EEFSAALLHLAKRRGVHNVNEVEEDTGNELSTKEQISRNSKALEEKYVAEL | |
| | | QLERLKKDGEVRGSINRFKTSDYVKEAKQLLKVQKAYHQLDQSFIDTYIDL | |
| | | LETRRTYYEGPGEGSPFGWKDIKEWYEMLMGHCTYFPEELRSVKYAYNA | |
| | | DLYNALNDLNNLVITRDENEKLEYYEKFQIIENVFKQKKKPTLKQIAKEILV | |
| | | NEEDIKGYRVTSTGKPEFTNLKVYHDIKDITARKEIIENAELLDQIAKILTIY | |
| | | QSSEDIQEELTNLNSELTQEEIEQISNLKGYTGTHNLSLKAINLILDELWHTN | |
| | | DNQIAIFNRLKLVPKKVDLSQQKEIPTTLVDDFILSPVVKRSFIQSIKVINAII | |
| | | KKYGLPNDIIIELAREKNSKDAQKMINEMQKRNRQTNERIEEIIRTTGKENA | |
| | | KYLIEKIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRISKTK | |
| | | KEYLLEERDINRFSVQKDFINRNLVDTRYATRGLMNLLRSYFRVNNLDVK | |
| | | VKSINGGFTSFLRRKWKFKKERNKGYKHHAEDALIIANADFIFKEWKKLD | |
| | | KAKKVMENQMFEEKQAESMPEIETEQEYKEIFITPHQIKHIKDFKDYKYSH | |
| | | RVDKKPNRKLINDTLYSTRKDDKGNTLIVNNLNGLYDKDNDKLKKLINKS | |
| | | PEKLLMYHHDPQTYQKLKLIMEQYGDEKNPLYKYYEETGNYLTKYSKKD | |
| | | NGPVIKKIKYYGNKLNAHLDITDDYPNSRNKVVKLSLKPYRFDVYLDNGV | |
| | | YKFVTVKNLDVIKKENYYEVNSKCYEEAKKLKKISNQAEFIASFYKNDLIK | |
| | | INGELYRVIGVNSDDRNIIELNMIDITYREYLENMNDKRPPHIIKTIASKTQSI | |
| | | KKYSTDILGNLYEVKSKKHPQIIKKG | |
| cCas9-v42 | Staphylococcus | MKRNYILGLDIGITSVGYGIIDYETRDVIDAGVRLFKEANVENNEGRRSKR | N580A |
| | aureus | GARRLKRRRRHRIQRVKKLLFDYNLLTDHSELSGINPYEARVKGLSQKLSE | |
| | | EEFSAALLHLAKRRGVHNVNEVEEDTGNELSTKEQISRNSKALEEKYVAEL | |
| | | QLERLKKDGEVRGSINRFKTSDYVKEAKQLLKVQKAYHQLDQSFIDTYIDL | |
| | | LETRRTYYEGPGEGSPFGWKDIKEWYEMLMGHCTYFPEELRSVKYAYNA | |
| | | DLYNALNDLNNLVITRDENEKLEYYEKFQIIENVFKQKKKPTLKQIAKEILV | |
| | | NEEDIKGYRVTSTGKPEFTNLKVYHDIKDITARKEIIENAELLDQIAKILTIY | |
| | | QSSEDIQEELTNLNSELTQEEIEQISNLKGYTGTHNLSLKAINLILDELWHTN | |
| | | DNQIAIFNRLKLVPKKVDLSQQKEIPTTLVDDFILSPVVKRSFIQSIKVINAII | |
| | | KKYGLPNDIIIELAREKNSKDAQKMINEMQKRNRQTNERIEEIIRTTGKENA | |
| | | KYLIEKIKLHDMQEGKCLYSLEAIPLEDLLNNPFNYEVDHIIPRSVSFDNSFN | |
| | | NKVLVKQEENSKKGNRTPFQYLSSSDSKISYETFKKHILNLAKGKGRISKTK | |
| | | KEYLLEERDINRFSVQKDFINRNLVDTRYATRGLMNLLRSYFRVNNLDVK | |
| | | VKSINGGFTSFLRRKWKFKKERNKGYKHHAEDALIIANADFIFKEWKKLD | |
| | | KAKKVMENQMFEEKQAESMPEIETEQEYKEIFITPHQIKHIKDFKDYKYSH | |
| | | RVDKKPNRKLINDTLYSTRKDDKGNTLIVNNLNGLYDKDNDKLKKLINKS | |
| | | PEKLLMYHHDPQTYQKLKLIMEQYGDEKNPLYKYYEETGNYLTKYSKKD | |
| | | NGPVIKKIKYYGNKLNAHLDITDDYPNSRNKVVKLSLKPYRFDVYLDNGV | |
| | | YKFVTVKNLDVIKKENYYEVNSKCYEEAKKLKKISNQAEFIASFYKNDLIK | |
| | | INGELYRVIGVNNNRLNKIELNMIDITYREYLENMNDKRPPHIIKTIASKTQS | |
| | | IKKYSTDILGNLYEVKSKKHPQIIKKG | |
| CdiCas9 | Corynebacterium | MKYHVGIDVGTFSVGLAAIEVDDAGMPIKTLSLVSHIHDSGLDPDEIKSAV | H573A |
| | diphtheriae | TRLASSGIARRTRRLYRRKRRRLQQLDKFIQRQGWPVIELEDYSDPLYPWK | (Alter- |
| | | VRAELAASYIADEKERGEKLSVALRHIARHRGWRNPYAKVSSLYLPDGPS | nate) |
| | | DAFKAIREEIKRASGQPVPETATVGQMVTLCELGTLKLRGEGGVLSARLQQ | |
| | | SDYAREIQEICRMQEIGQELYRKIIDVVFAAESPKGSASSRVGKDPLQPGKN | |
| | | RALKASDAFQRYRIAALIGNLRVRVDGEKRILSVEEKNLVFDHLVNLTPKK | |
| | | EPEWVTIAEILGIDRGQLIGTATMTDDGERAGARPPTHDTNRSIVNSRIAPL | |
| | | VDWWKTASALEQHAMVKALSNAEVDDFDSPEGAKVQAFFADLDDDVHA | |
| | | KLDSLHLPVGRAAYSEDTLVRLTRRMLSDGVDLYTARLQEFGIEPSWTPPT | |
| | | PRIGEPVGNPAVDRVLKTVSRWLESATKTWGAPERVIIEHVREGFVTEKRA | |
| | | REMDGDMRRRAARNAKLFQEMQEKLNVQGKPSRADLWRYQSVQRQNC | |
| | | QCAYCGSPITFSNSEMDHIVPRAGQGSTNTRENLVAVCHRCNQSKGNTPFA | |
| | | IWAKNTSIEGVSVKEAVERTRHWVTDTGMRSTDFKKFTKAVVERFQRAT | |
| | | MDEEIDARSMESVAWMANELRSRVAQHFASHGTTVRVYRGSLTAEARRA | |
| | | SGISGKLKFFDGVGKSRLDRRHHAIDAAVIAFTSDYVAETLAVRSNLKQSQ | |
| | | AHRQEAPQWREFTGKDAEHRAAWRVWCQKMEKLSALLTEDLRDDRVVV | |
| | | MSNVRLRLGNGSAHKETIGKLSKVKLSSQLSVSDIDKASSEALWCALTREP | |
| | | GFDPKEGLPANPERHIRVNGTHVYAGDNIGLFPVSAGSIALRGGYAELGSSF | |
| | | HHARVYKITSGKKPAFAMLRVYTIDLLPYRNQDLFSVELKPQTMSMRQAE | |
| | | KKLRDALATGNAEYLGWLVVDDELVVDTSKIATDQVKAVEAELGTIRRW | |
| | | RVDGFFSPSKLRLRPLQMSKEGIKKESAPELSKIIDRPGWLPAVNKLFSDGN | |
| | | VTVVRRDSLGRVRLESTAHLPVTWKVQ | |
| CjeCas9 | Campylobacter | MARILAFDIGISSIGWAFSENDELKDCGVRIFTKVENPKTGESLALPRRLARS | N582A |
| | jejuni | ARKRLARRKARLNHLKHLIANEFKLNYEDYQSFDESLAKAYKGSLISPYEL | |
| | | RFRALNELLSKQDFARVILHIAKRRGYDDIKNSDDKEKGAILKAIKQNEEK | |
| | | LANYQSVGEYLYKEYFQKFKENSKEFTNVRNKKESYERCIAQSFLKDELKL | |
| | | IFKKQREFGFSFSKKFEEEVLSVAFYKRALKDFSHLVGNCSFFTDEKRAPKN | |
| | | SPLAFMFVALTRIINLLNNLKNTEGILYTKDDLNALLNEVLKNGTLTYKQT | |
| | | KKLLGLSDDYEFKGEKGTYFIEFKKYKEFIKALGEHNLSQDDLNEIAKDITL | |
| | | IKDEIKLKKALAKYDLNQNQIDSLSKLEFKDHLNISFKALKLVTPLMLEGK | |
| | | KYDEACNELNLKVAINEDKKDFLPAFNETYYKDEVTNPVVLRAIKEYRKV | |
| | | LNALLKKYGKVHKINIELAREVGKNHSQRAKIEKEQNENYKAKKDAELEC | |
| | | EKLGLKINSKNILKLRLFKEQKEFCAYSGEKIKISDLQDEKMLEIDHIYPYSR | |
| | | SFDDSYMNKVLVFTKQNQEKLNQTPFEAFGNDSAKWQKIEVLAKNLPTKK | |
| | | QKRILDKNYKDKEQKNFKDRNLNDTRYIARLVLNYTKDYLDFLPLSDDEN | |
| | | TKLNDTQKGSKVHVEAKSGMLTSALRHTWGFSAKDRNNHLHHAIDAVIIA | |
| | | YANNSIVKAFSDFKKEQESNSAELYAKKISELDYKNKRKFFEPFSGFRQKV | |
| | | LDKIDEIFVSKPERKKPSGALHEETFRKEEEFYQSYGGKEGVLKALELGKIR | |
| | | KVNGKIVKNGDMFRVDIFKHKKTNKFYAVPIYTMDFALKVLPNKAVARS | |
| | | KKGEIKDWILMDENYEFCFSLYKDSLILIQTKDMQEPEFVYYNAFTSSTVSL | |
| | | IVSKHDNKFETLSKNQKILFKNANEKEVIAKSIGIQNLKVFEKYIVSALGEV | |
| | | TKAEFRQREDFKK | |
| GeoCas9 | Geobacillus | MRYKIGLDIGITSVGWAVMNLDIPRIEDLGVRIFDRAENPQTGESLALPRRL | N605A |
| | stearothermo- | ARSARRRLRRRKHRLERIRRLVIREGILTKEELDKLFEEKHEIDVWQLRVEA | |
| | philus | LDRKLNNDELARVLLHLAKRRGFKSNRKSERSNKENSTMLKHIEENRAILS | |
| | | SYRTVGEMIVKDPKFALHKRNKGENYTNTIARDDLEREIRLIFSKQREFGN | |
| | | MSCTEEFENEYITIWASQRPVASKDDIEKKVGFCTFEPKEKRAPKATYTFQS | |
| | | FIAWEHINKLRLISPSGARGLTDEERRLLYEQAFQKNKITYHDIRTLLHLPD | |
| | | DTYFKGIVYDRGESRKQNENIRFLELDAYHQIRKAVDKVYGKGKSSSFLPI | |
| | | DFDTFGYALTLFKDDADIHSYLRNEYEQNGKRMPNLANKVYDNELIEELL | |
| | | NLSFTKFGHLSLKALRSILPYMEQGEVYSSACERAGYTFTGPKKKQKTMLL | |
| | | PNIPPIANPVVMRALTQARKVVNAIIKKYGSPVSIHIELARDLSQTFDERRKT | |
| | | KKEQDENRKKNETAIRQLMEYGLTLNPTGHDIVKFKLWSEQNGRCAYSLQ | |
| | | PIEIERLLEPGYVEVDHVIPYSRSLDDSYTNKVLVLTRENREKGNRIPAEYL | |
| | | GVGTERWQQFETFVLTNKQFSKKKRDRLLRLHYDENEETEFKNRNLNDTR | |
| | | YISRFFANFIREHLKFAESDDKQKVYTVNGRVTAHLRSRWEFNKNREESDL | |
| | | HHAVDAVIVACTTPSDIAKVTAFYQRREQNKELAKKTEPHFPQPWPHFAD | |
| | | ELRARLSKHPKESIKALNLGNYDDQKLESLQPVFVSRMPKRSVTGAAHQE | |
| | | TLRRYVGIDERSGKIQTVVKTKLSEIKLDASGHFPMYGKESDPRTYEAIRQR | |
| | | LLEHNNDPKKAFQEPLYKPKKNGEPGPVIRTVKIIDTKNQVIPLNDGKTVA | |
| | | YNSNIVRVDVFEKDGKYYCVPVYTMDIMKGILPNKAIEPNKPYSEWKEMT | |
| | | EDYTFRFSLYPNDLIRIELPREKTVKTAAGEEINVKDVFVYYKTIDSANGGL | |
| | | ELISHDHRFSLRGVGSRTLKRFEKYQVDVLGNIYKVRGEKRVGLASSAHSK | |
| | | PGKTIRPLQSTRD | |
| iSpyMacCas9 | Streptococcus | MDKKYSIGLDIGTNSVGWAVITDEYKVPSKKFKVLGNTDRHSIKKNLIGAL | N863A |
| | spp. | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRKLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLKREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEIQTVGQNGGLFDDNPKSPLEVTPS | |
| | | KLVPLKKELNPKKYGGYQKPTTAYPVLLITDTKQLIPISVMNKKQFEQNPV | |
| | | KFLRDRGYQQVGKNDFIKLPKYTLVDIGDGIKRLWASSKEIHKGNQLVVS | |
| | | KKSQILLYHAHHLDSDLSNDYLQNHNQQFDVLFNEIISFSKKCKLGKEHIQ | |
| | | KIENVYSNKKNSASIEELAESFIKLLGFTQLGATSPFNFLGVKLNQKQYKGK | |
| | | KDYILPCTEGTLIRQSITGLYETRVDLSKIGEDSGGSGGSKRTADGSEFES | |
| NmeCas9 | Neisseria | MAAFKPNSINYILGLDIGIASVGWAMVEIDEEENPIRLIDLGVRVFERAEVP | N611A |
| | meningitidis | KTGDSLAMARRLARSVRRLTRRRAHRLLRTRRLLKREGVLQAANFDENGL | |
| | | IKSLPNTPWQLRAAALDRKLTPLEWSAVLLHLIKHRGYLSQRKNEGETAD | |
| | | KELGALLKGVAGNAHALQTGDFRTPAELALNKFEKESGHIRNQRSDYSHT | |
| | | FSRKDLQAELILLFEKQKEFGNPHVSGGLKEGIETLLMTQRPALSGDAVQK | |
| | | MLGHCTFEPAEPKAAKNTYTAERFIWLTKLNNLRILEQGSERPLTDTERAT | |
| | | LMDEPYRKSKLTYAQARKLLGLEDTAFFKGLRYGKDNAEASTLMEMKAY | |
| | | HAISRALEKEGLKDKKSPLNLSPELQDEIGTAFSLFKTDEDITGRLKDRIQPE | |
| | | ILEALLKHISFDKFVQISLKALRRIVPLMEQGKRYDEACAEIYGDHYGKKNT | |
| | | EEKIYLPPIPADEIRNPVVLRALSQARKVINGVVRRYGSPARIHIETAREVGK | |
| | | SFKDRKEIEKRQEENRKDREKAAAKFREYFPNFVGEPKSKDILKLRLYEQQ | |
| | | HGKCLYSGKEINLGRLNEKGYVEIDHALPFSRTWDDSFNNKVLVLGSENQ | |
| | | NKGNQTPYEYFNGKDNSREWQEFKARVETSRFPRSKKQRILLQKFDEDGF | |
| | | KERNLNDTRYVNRFLCQFVADRMRLTGKGKKRVFASNGQITNLLRGFWG | |
| | | LRKVRAENDRHHALDAVVVACSTVAMQQKITRFVRYKEMNAFDGKTIDK | |
| | | ETGEVLHQKTHFPQPWEFFAQEVMIRVFGKPDGKPEFEEADTLEKLRTLLA | |
| | | EKLSSRPEAVHEYVTPLFVSRAPNRKMSGQGHMETVKSAKRLDEGVSVLR | |
| | | VPLTQLKLKDLEKMVNREREPKLYEALKARLEAHKDDPAKAFAEPFYKY | |
| | | DKAGNRTQQVKAVRVEQVQKTGVWVRNHNGIADNATMVRVDVFEKGD | |
| | | KYYLVPIYSWQVAKGILPDRAVVQGKDEEDWQLIDDSFNFKFSLHPNDLV | |
| | | EVITKKARMFGYFASCHRGTGNINIRIHDLDHKIGKNGILEGIGVKTALSFQ | |
| | | KYQIDELGKEIRPCRLKKRPPVR | |
| ScaCas9 | Streptococcus | MEKKYSIGLDIGTNSVGWAVITDDYKVPSKKFKVLGNTNRKSIKKNLMGA | N872A |
| | canis | LLFDSGETAEATRLKRTARRRYTRRKNRIRYLQEIFANEMAKLDDSFFQRL | |
| | | EESFLVEEDKKNERHPIFGNLADEVAYHRNYPTIYHLRKKLADSPEKADLR | |
| | | LIYLALAHIIKFRGHFLIEGKLNAENSDVAKLFYQLIQTYNQLFEESPLDEIE | |
| | | VDAKGILSARLSKSKRLEKLIAVFPNEKKNGLFGNIIALALGLTPNFKSNFD | |
| | | LTEDAKLQLSKDTYDDDLDELLGQIGDQYADLFSAAKNLSDAILLSDILRS | |
| | | NSEVTKAPLSASMVKRYDEHHQDLALLKTLVRQQFPEKYAEIFKDDTKNG | |
| | | YAGYVGIGIKHRKRTTKLATQEEFYKFIKPILEKMDGAEELLAKLNRDDLL | |
| | | RKQRTFDNGSIPHQIHLKELHAILRRQEEFYPFLKENREKIEKILTFRIPYYV | |
| | | GPLARGNSRFAWLTRKSEEAITPWNFEEVVDKGASAQSFIERMTNFDEQLP | |
| | | NKKVLPKHSLLYEYFTVYNELTKVKYVTERMRKPEFLSGEQKKAIVDLLF | |
| | | KTNRKVTVKQLKEDYFKKIECFDSVEIIGVEDRFNASLGTYHDLLKIIKDKD | |
| | | FLDNEENEDILEDIVLTLTLFEDREMIEERLKTYAHLFDDKVMKQLKRRHY | |
| | | TGWGRLSRKMINGIRDKQSGKTILDFLKSDGFSNRNFMQLIHDDSLTFKEEI | |
| | | EKAQVSGQGDSLHEQIADLAGSPAIKKGILQTVKIVDELVKVMGHKPENIV | |
| | | IEMARENQTTTKGLQQSRERKKRIEEGIKELESQILKENPVENTQLQNEKLY | |
| | | LYYLQNGRDMYVDQELDINRLSDYDVDHIVPQSFIKDDSIDNKVLTRSVEN | |
| | | RGKSDNVPSEEVVKKMKNYWRQLLNAKLITQRKFDNLTKAERGGLSEAD | |
| | | KAGFIKRQLVETRQITKHVARILDSRMNTKRDKNDKPIREVKVITLKSKLVS | |
| | | DFRKDFQLYKVRDINNYHHAHDAYLNAVVGTALIKKYPKLESEFVYGDY | |
| | | KVYDVRKMIAKSEQEIGKATAKRFFYSNIMNFFKTEVKLANGEIRKRPLIET | |
| | | NGETGEVVWNKEKDFATVRKVLAMPQVNIVKKTEVQTGGFSKESILSKRE | |
| | | SAKLIPRKKGWDTRKYGGFGSPTVAYSILVVAKVEKGKAKKLKSVKVLVG | |
| | | ITIMEKGSYEKDPIGFLEAKGYKDIKKELIFKLPKYSLFELENGRRRMLASA | |
| | | TELQKANELVLPQHLVRLLYYTQNISATTGSNNLGYIEQHREEFKEIFEKIID | |
| | | FSEKYILKNKVNSNLKSSFDEQFAVSDSILLSNSFVSLLKYTSFGASGGFTFL | |
| | | DLDVKQGRLRYQTVTEVLDATLIYQSITGLYETRTDLSQLGGD | |
| ScaCas9- | Streptococcus | MEKKYSIGLDIGTNSVGWAVITDDYKVPSKKFKVLGNTNRKSIKKNLMGA | N872A |
| HiFi-Sc++ | canis | LLFDSGETAEATRLKRTARRRYTRRKNRIRYLQEIFANEMAKLDDSFFQRL | |
| | | EESFLVEEDKKNERHPIFGNLADEVAYHRNYPTIYHLRKKLADSPEKADLR | |
| | | LIYLALAHIIKFRGHFLIEGKLNAENSDVAKLFYQLIQTYNQLFEESPLDEIE | |
| | | VDAKGILSARLSKSKRLEKLIAVFPNEKKNGLFGNIIALALGLTPNFKSNFD | |
| | | LTEDAKLQLSKDTYDDDLDELLGQIGDQYADLFSAAKNLSDAILLSDILRS | |
| | | NSEVTKAPLSASMVKRYDEHHQDLALLKTLVRQQFPEKYAEIFKDDTKNG | |
| | | YAGYVGADKKLRKRSGKLATEEEFYKFIKPILEKMDGAEELLAKLNRDDL | |
| | | LRKQRTFDNGSIPHQIHLKELHAILRRQEEFYPFLKENREKIEKILTFRIPYYV | |
| | | GPLARGNSRFAWLTRKSEEAITPWNFEEVVDKGASAQSFIERMTNFDEQLP | |
| | | NKKVLPKHSLLYEYFTVYNELTKVKYVTERMRKPEFLSGEQKKAIVDLLF | |
| | | KTNRKVTVKQLKEDYFKKIECFDSVEIIGVEDRFNASLGTYHDLLKIIKDKD | |
| | | FLDNEENEDILEDIVLTLTLFEDREMIEERLKTYAHLFDDKVMKQLKRRHY | |
| | | TGWGRLSRKMINGIRDKQSGKTILDFLKSDGFSNANFMQLIHDDSLTFKEEI | |
| | | EKAQVSGQGDSLHEQIADLAGSPAIKKGILQTVKIVDELVKVMGHKPENIV | |
| | | IEMARENQTTTKGLQQSRERKKRIEEGIKELESQILKENPVENTQLQNEKLY | |
| | | LYYLQNGRDMYVDQELDINRLSDYDVDHIVPQSFIKDDSIDNKVLTRSVEN | |
| | | RGKSDNVPSEEVVKKMKNYWRQLLNAKLITQRKFDNLTKAERGGLSEAD | |
| | | KAGFIKRQLVETRQITKHVARILDSRMNTKRDKNDKPIREVKVITLKSKLVS | |
| | | DFRKDFQLYKVRDINNYHHAHDAYLNAVVGTALIKKYPKLESEFVYGDY | |
| | | KVYDVRKMIAKSEQEIGKATAKRFFYSNIMNFFKTEVKLANGEIRKRPLIET | |
| | | NGETGEVVWNKEKDFATVRKVLAMPQVNIVKKTEVQTGGFSKESILSKRE | |
| | | SAKLIPRKKGWDTRKYGGFGSPTVAYSILVVAKVEKGKAKKLKSVKVLVG | |
| | | ITIMEKGSYEKDPIGFLEAKGYKDIKKELIFKLPKYSLFELENGRRRMLASA | |
| | | KELQKANELVLPQHLVRLLYYTQNISATTGSNNLGYIEQHREEFKEIFEKIID | |
| | | FSEKYILKNKVNSNLKSSFDEQFAVSDSILLSNSFVSLLKYTSFGASGGFTFL | |
| | | DLDVKQGRLRYQTVTEVLDATLIYQSITGLYETRTDLSQLGGD | |
| SpyCas9- | Streptococcus | MDKKYSIGLDIGTNSVGWAVITDEYKVPSKKFKVLGNTDRHSIKKNLIGAL | N863A |
| 3var-NRRH | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMVKRYDEHHQDLTLLKALVRQQLPEKYKEIFFDQSKNGY | |
| | | AGYIDGGASQEEFYKFIKPILEKMDGTEELLVKLNREDLLRKQRTFDNGIIP | |
| | | HQIHLGELHAILRRQGDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRLRYTGWGRLSRKLIN | |
| | | GIRDKQSGKTILDFLKSDGFANRNFMQLIHDDSLTFKEDIQKAQVSGQGDS | |
| | | LHEHIANLAGSPAIKKGILQTVKVVDELVKVMGGHKPENIVIEMARENQTT | |
| | | QKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKGNSDKLIARK | |
| | | KDWDPKKYGGFNSPTAAYSVLVVAKVEKGKSKKLKSVKELLGITIMERSS | |
| | | FEKNPIGFLEAKGYKEVKKDLIIKLPKYSLFELENGRKRMLASAGVLHKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGVPAAFKYF | |
| | | DTTIDKKRYTSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| 3var-NRTH | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMVKRYDEHHQDLTLLKALVRQQLPEKYKEIFFDQSKNGY | |
| | | AGYIDGGASQEEFYKFIKPILEKMDGTEELLVKLNREDLLRKQRTFDNGIIP | |
| | | HQIHLGELHAILRRQGDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRLRYTGWGRLSRKLIN | |
| | | GIRDKQSGKTILDFLKSDGFANRNFMQLIHDDSLTFKEDIQKAQVSGQGDS | |
| | | LHEHIANLAGSPAIKKGILQTVKVVDELVKVMGGHKPENIVIEMARENQTT | |
| | | QKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKGNSDKLIARK | |
| | | KDWDPKKYGGFNSPTVAYSVLVVAKVEKGKSKKLKSVKELLGITIMERSS | |
| | | FEKNPIGFLEAKGYKEVKKDLIIKLPKYSLFELENGRKRMLASASVLHKGN | |
| | | ELALPSKYVNFLYLASHYEKLKGSSEDNKQKQLFVEQHKHYLDEIIEQISEF | |
| | | SKRVILADANLDKVLSAYNKHRDKPIREQAENIIHLFTLTNLGASAAFKYF | |
| | | DTTIGRKLYTSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| 3var-NRCH | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMVKRYDEHHQDLTLLKALVRQQLPEKYKEIFFDQSKNGY | |
| | | AGYIDGGASQEEFYKFIKPILEKMDGTEELLVKLNREDLLRKQRTFDNGIIP | |
| | | HQIHLGELHAILRRQGDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRLRYTGWGRLSRKLIN | |
| | | GIRDKQSGKTILDFLKSDGFANRNFMQLIHDDSLTFKEDIQKAQVSGQGDS | |
| | | LHEHIANLAGSPAIKKGILQTVKVVDELVKVMGGHKPENIVIEMARENQTT | |
| | | QKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKGNSDKLIARK | |
| | | KDWDPKKYGGFNSPTVAYSVLVVAKVEKGKSKKLKSVKELLGITIMERSS | |
| | | FEKNPIDFLEAKGYKEVKKDLIIKLPKYSLFELENGRKRMLASAGVLQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTINRKQYNTTKEVLDATLIRQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| HF1 | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFDSPTVAYSVLVVAKVEKGKSKKLKSVKELLGITIMERSS | |
| | | FEKNPIDFLEAKGYKEVKKDLIIKLPKYSLFELENGRKRMLASAGELQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTIDRKRYTSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| QQR1 | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFDSPTVAYSVLVVAKVEKGKSKKLKSVKELLGITIMERSS | |
| | | FEKNPIDFLEAKGYKEVKKDLIIKLPKYSLFELENGRKRMLASARELQKGN | |
| | | ELALPSKYVNFLYLASHYEKLKGSPEDNEQKQLFVEQHKHYLDEIIEQISEF | |
| | | SKRVILADAQLDKVLSAYNKHRDKPIREQAENIIHLFTLTNLGAPAAFKYF | |
| | | DTTFKQKQYRSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| SpG | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFLWPTVAYSVLVVAKVEKGKSKKLKSVKELLGITIMERSS | |
| | | FEKNPIDFLEAKGYKEVKKDLIIKLPKYSLFELENGRKRMLASAKQLQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTIDRKQYRSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| VQR | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFVSPTVAYSVLVVAKVEKGKSKKLKSVKELLGITIMERSS | |
| | | FEKNPIDFLEAKGYKEVKKDLIIKLPKYSLFELENGRKRMLASAGELQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTIDRKQYRSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| VRER | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDAKLQLSKDTYDDDLDNLLAQIGDQYADLFLAAKNLSDAILLSDILRV | |
| | | NTEITKAPLSASMIKRYDEHHQDLTLLKALVRQQLPEKYKEIFFDQSKNGY | |
| | | AGYIDGGASQEEFYKFIKPILEKMDGTEELLVKLNREDLLRKQRTFDNGSIP | |
| | | HQIHLGELHAILRRQEDFYPFLKDNREKIEKILTFRIPYYVGPLARGNSRFA | |
| | | WMTRKSEETITPWNFEEVVDKGASAQSFIERMTNFDKNLPNEKVLPKHSLL | |
| | | YEYFTVYNELTKVKYVTEGMRKPAFLSGEQKKAIVDLLFKTNRKVTVKQL | |
| | | KEDYFKKIECFDSVEISGVEDRFNASLGTYHDLLKIIKDKDFLDNEENEDILE | |
| | | DIVLTLTLFEDREMIEERLKTYAHLFDDKVMKQLKRRRYTGWGRLSRKLI | |
| | | NGIRDKQSGKTILDFLKSDGFANRNFMQLIHDDSLTFKEDIQKAQVSGQGD | |
| | | SLHEHIANLAGSPAIKKGILQTVKVVDELVKVMGRHKPENIVIEMARENQT | |
| | | TQKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFVSPTVAYSVLVVAKVEKGKSKKLKSVKELLGITIMERSS | |
| | | FEKNPIDFLEAKGYKEVKKDLIIKLPKYSLFELENGRKRMLASARELQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTIDRKEYRSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| xCas | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDTKLQLSKDTYDDDLDNLLAQIGDQYADLFLAAKNLSDAILLSDILRV | |
| | | NTEITKAPLSASMIKLYDEHHQDLTLLKALVRQQLPEKYKEIFFDQSKNGY | |
| | | AGYIDGGASQEEFYKFIKPILEKMDGTEELLVKLNREDLLRKQRTFDNGIIP | |
| | | HQIHLGELHAILRRQEDFYPFLKDNREKIEKILTFRIPYYVGPLARGNSRFA | |
| | | WMTRKSEETITPWNFEKVVDKGASAQSFIERMTNFDKNLPNEKVLPKHSL | |
| | | LYEYFTVYNELTKVKYVTEGMRKPAFLSGDQKKAIVDLLFKTNRKVTVKQ | |
| | | LKEDYFKKIECFDSVEISGVEDRFNASLGTYHDLLKIIKDKDFLDNEENEDIL | |
| | | EDIVLTLTLFEDREMIEERLKTYAHLFDDKVMKQLKRRRYTGWGRLSRKLI | |
| | | NGIRDKQSGKTILDFLKSDGFANRNFIQLIHDDSLTFKEDIQKAQVSGQGDS | |
| | | LHEHIANLAGSPAIKKGILQTVKVVDELVKVMGRHKPENIVIEMARENQTT | |
| | | QKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESILPKRNSDKLIARK | |
| | | KDWDPKKYGGFDSPTVAYSVLVVAKVEKGKSKKLKSVKELLGITIMERSS | |
| | | FEKNPIDFLEAKGYKEVKKDLIIKLPKYSLFELENGRKRMLASAGVLQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPAAFKYF | |
| | | DTTIDRKRYTSTKEVLDATLIHQSITGLYETRIDLSQLGGD | |
| SpyCas9- | Streptococcus | MDKKYSIGLDIGTNSVGWAVITDEYKVPSKKFKVLGNTDRHSIKKNLIGAL | N863A |
| xCas-NG | pyogenes | LFDSGETAEATRLKRTARRRYTRRKNRICYLQEIFSNEMAKVDDSFFHRLE | |
| | | ESFLVEEDKKHERHPIFGNIVDEVAYHEKYPTIYHLRKKLVDSTDKADLRLI | |
| | | YLALAHMIKFRGHFLIEGDLNPDNSDVDKLFIQLVQTYNQLFEENPINASG | |
| | | VDAKAILSARLSKSRRLENLIAQLPGEKKNGLFGNLIALSLGLTPNFKSNFD | |
| | | LAEDTKLQLSKDTYDDDLDNLLAQIGDQYADLFLAAKNLSDAILLSDILRV | |
| | | NTEITKAPLSASMIKLYDEHHQDLTLLKALVRQQLPEKYKEIFFDQSKNGY | |
| | | AGYIDGGASQEEFYKFIKPILEKMDGTEELLVKLNREDLLRKQRTFDNGIIP | |
| | | HQIHLGELHAILRRQEDFYPFLKDNREKIEKILTFRIPYYVGPLARGNSRFA | |
| | | WMTRKSEETITPWNFEKVVDKGASAQSFIERMTNFDKNLPNEKVLPKHSL | |
| | | LYEYFTVYNELTKVKYVTEGMRKPAFLSGDQKKAIVDLLFKTNRKVTVKQ | |
| | | LKEDYFKKIECFDSVEISGVEDRFNASLGTYHDLLKIIKDKDFLDNEENEDIL | |
| | | EDIVLTLTLFEDREMIEERLKTYAHLFDDKVMKQLKRRRYTGWGRLSRKLI | |
| | | NGIRDKQSGKTILDFLKSDGFANRNFIQLIHDDSLTFKEDIQKAQVSGQGDS | |
| | | LHEHIANLAGSPAIKKGILQTVKVVDELVKVMGRHKPENIVIEMARENQTT | |
| | | QKGQKNSRERMKRIEEGIKELGSQILKEHPVENTQLQNEKLYLYYLQNGR | |
| | | DMYVDQELDINRLSDYDVDHIVPQSFLKDDSIDNKVLTRSDKNRGKSDNV | |
| | | PSEEVVKKMKNYWRQLLNAKLITQRKFDNLTKAERGGLSELDKAGFIKRQ | |
| | | LVETRQITKHVAQILDSRMNTKYDENDKLIREVKVITLKSKLVSDFRKDFQ | |
| | | FYKVREINNYHHAHDAYLNAVVGTALIKKYPKLESEFVYGDYKVYDVRK | |
| | | MIAKSEQEIGKATAKYFFYSNIMNFFKTEITLANGEIRKRPLIETNGETGEIV | |
| | | WDKGRDFATVRKVLSMPQVNIVKKTEVQTGGFSKESIRPKRNSDKLIARK | |
| | | KDWDPKKYGGFVSPTVAYSVLVVAKVEKGKSKKLKSVKELLGITIMERSS | |
| | | FEKNPIDFLEAKGYKEVKKDLIIKLPKYSLFELENGRKRMLASARFLQKGN | |
| | | ELALPSKYVNFLYLASHYEKLKGSPEDNEQKQLFVEQHKHYLDEIIEQISEF | |
| | | SKRVILADANLDKVLSAYNKHRDKPIREQAENIIHLFTLTNLGAPRAFKYFD | |
| | | TTIDRKVYRSTKEVLDATLIHQSITGLYETRIDLSQLGGD | |
| St1Cas9- | Streptococcus | MSDLVLGLDIGIGSVGVGILNKVTGEIIHKNSRIFPAAQAENNLVRRTNRQG | N622A |
| CNRZ1066 | thermophilus | RRLARRKKHRRVRLNRLFEESGLITDFTKISINLNPYQLRVKGLTDELSNEE | |
| | | LFIALKNMVKHRGISYLDDASDDGNSSVGDYAQIVKENSKQLETKTPGQIQ | |
| | | LERYQTYGQLRGDFTVEKDGKKHRLINVFPTSAYRSEALRILQTQQEFNPQI | |
| | | TDEFINRYLEILTGKRKYYHGPGNEKSRTDYGRYRTSGETLDNIFGILIGKC | |
| | | TFYPDEFRAAKASYTAQEFNLLNDLNNLTVPTETKKLSKEQKNQIINYVKN | |
| | | EKAMGPAKLFKYIAKLLSCDVADIKGYRIDKSGKAEIHTFEAYRKMKTLET | |
| | | LDIEQMDRETLDKLAYVLTLNTEREGIQEALEHEFADGSFSQKQVDELVQF | |
| | | RKANSSIFGKGWHNFSVKLMMELIPELYETSEEQMTILTRLGKQKTTSSSN | |
| | | KTKYIDEKLLTEEIYNPVVAKSVRQAIKIVNAAIKEYGDFDNIVIEMARETN | |
| | | EDDEKKAIQKIQKANKDEKDAAMLKAANQYNGKAELPHSVFHGHKQLAT | |
| | | KIRLWHQQGERCLYTGKTISIHDLINNSNQFEVDHILPLSITFDDSLANKVLV | |
| | | YATANQEKGQRTPYQALDSMDDAWSFRELKAFVRESKTLSNKKKEYLLT | |
| | | EEDISKFDVRKKFIERNLVDTRYASRVVLNALQEHFRAHKIDTKVSVVRGQ | |
| | | FTSQLRRHWGIEKTRDTYHHHAVDALIIAASSQLNLWKKQKNTLVSYSEE | |
| | | QLLDIETGELISDDEYKESVFKAPYQHFVDTLKSKEFEDSILFSYQVDSKFN | |
| | | RKISDATIYATRQAKVGKDKKDETYVLGKIKDIYTQDGYDAFMKIYKKDK | |
| | | SKFLMYRHDPQTFEKVIEPILENYPNKQMNEKGKEVPCNPFLKYKEEHGYI | |
| | | RKYSKKGNGPEIKSLKYYDSKLLGNPIDITPENSKNKVVLQSLKPWRTDVY | |
| | | FNKATGKYEILGLKYADLQFEKGTGTYKISQEKYNDIKKKEGVDSDSEFKF | |
| | | TLYKNDLLLVKDTETKEQQLFRFLSRTLPKQKHYVELKPYDKQKFEGGEA | |
| | | LIKVLGNVANGGQCIKGLAKSNISIYKVRTDVLGNQHIIKNEGDKPKLDF | |
| St1Cas9- | Streptococcus | MSDLVLGLDIGIGSVGVGILNKVTGEIIHKNSRIFPAAQAENNLVRRTNRQG | N622A |
| LMG1831 | thermophilus | RRLARRKKHRRVRLNRLFEESGLITDFTKISINLNPYQLRVKGLTDELSNEE | |
| | | LFIALKNMVKHRGISYLDDASDDGNSSVGDYAQIVKENSKQLETKTPGQIQ | |
| | | LERYQTYGQLRGDFTVEKDGKKHRLINVFPTSAYRSEALRILQTQQEFNPQI | |
| | | TDEFINRYLEILTGKRKYYHGPGNEKSRTDYGRYRTSGETLDNIFGILIGKC | |
| | | TFYPDEFRAAKASYTAQEFNLLNDLNNLTVPTETKKLSKEQKNQIINYVKN | |
| | | EKAMGPAKLFKYIAKLLSCDVADIKGYRIDKSGKAEIHTFEAYRKMKTLET | |
| | | LDIEQMDRETLDKLAYVLTLNTEREGIQEALEHEFADGSFSQKQVDELVQF | |
| | | RKANSSIFGKGWHNFSVKLMMELIPELYETSEEQMTILTRLGKQKTTSSSN | |
| | | KTKYIDEKLLTEEIYNPVVAKSVRQAIKIVNAAIKEYGDFDNIVIEMARETN | |
| | | EDDEKKAIQKIQKANKDEKDAAMLKAANQYNGKAELPHSVFHGHKQLAT | |
| | | KIRLWHQQGERCLYTGKTISIHDLINNSNQFEVDHILPLSITFDDSLANKVLV | |
| | | YATANQEKGQRTPYQALDSMDDAWSFRELKAFVRESKTLSNKKKEYLLT | |
| | | EEDISKFDVRKKFIERNLVDTRYASRVVLNALQEHFRAHKIDTKVSVVRGQ | |
| | | FTSQLRRHWGIEKTRDTYHHHAVDALIIAASSQLNLWKKQKNTLVSYSEE | |
| | | QLLDIETGELISDDEYKESVFKAPYQHFVDTLKSKEFEDSILFSYQVDSKFN | |
| | | RKISDATIYATRQAKVGKDKKDETYVLGKIKDIYTQDGYDAFMKIYKKDK | |
| | | SKFLMYRHDPQTFEKVIEPILENYPNKQMNEKGKEVPCNPFLKYKEEHGYI | |
| | | RKYSKKGNGPEIKSLKYYDSKLLGNPIDITPENSKNKVVLQSLKPWRTDVY | |
| | | FNKNTGKYEILGLKYADLQFEKKTGTYKISQEKYNGIMKEEGVDSDSEFKF | |
| | | TLYKNDLLLVKDTETKEQQLFRFLSRTMPNVKYYVELKPYSKDKFEKNES | |
| | | LIEILGSADKSGRCIKGLGKSNISIYKVRTDVLGNQHIIKNEGDKPKLDF | |
| St1Cas9- | Streptococcus | MSDLVLGLDIGIGSVGVGILNKVTGEIIHKNSRIFPAAQAENNLVRRTNRQG | N622A |
| MTH17CL39 | thermophilus | RRLARRKKHRRVRLNRLFEESGLITDFTKISINLNPYQLRVKGLTDELSNEE | |
| 6 | | LFIALKNMVKHRGISYLDDASDDGNSSVGDYAQIVKENSKQLETKTPGQIQ | |
| | | LERYQTYGQLRGDFTVEKDGKKHRLINVFPTSAYRSEALRILQTQQEFNPQI | |
| | | TDEFINRYLEILTGKRKYYHGPGNEKSRTDYGRYRTSGETLDNIFGILIGKC | |
| | | TFYPDEFRAAKASYTAQEFNLLNDLNNLTVPTETKKLSKEQKNQIINYVKN | |
| | | EKAMGPAKLFKYIAKLLSCDVADIKGYRIDKSGKAEIHTFEAYRKMKTLET | |
| | | LDIEQMDRETLDKLAYVLTLNTEREGIQEALEHEFADGSFSQKQVDELVQF | |
| | | RKANSSIFGKGWHNFSVKLMMELIPELYETSEEQMTILTRLGKQKTTSSSN | |
| | | KTKYIDEKLLTEEIYNPVVAKSVRQAIKIVNAAIKEYGDFDNIVIEMARETN | |
| | | EDDEKKAIQKIQKANKDEKDAAMLKAANQYNGKAELPHSVFHGHKQLAT | |
| | | KIRLWHQQGERCLYTGKTISIHDLINNSNQFEVDHILPLSITFDDSLANKVLV | |
| | | YATANQEKGQRTPYQALDSMDDAWSFRELKAFVRESKTLSNKKKEYLLT | |
| | | EEDISKFDVRKKFIERNLVDTRYASRVVLNALQEHFRAHKIDTKVSVVRGQ | |
| | | FTSQLRRHWGIEKTRDTYHHHAVDALIIAASSQLNLWKKQKNTLVSYSED | |
| | | QLLDIETGELISDDEYKESVFKAPYQHFVDTLKSKEFEDSILFSYQVDSKFN | |
| | | RKISDATIYATRQAKVGKDKADETYVLGKIKDIYTQDGYDAFMKIYKKDK | |
| | | SKFLMYRHDPQTFEKVIEPILENYPNKQINEKGKEVPCNPFLKYKEEHGYIR | |
| | | KYSKKGNGPEIKSLKYYDSKLGNHIDITPKDSNNKVVLQSLKPWRTDVYF | |
| | | NKNTGKYEILGLKYSDMQFEKGTGKYSISKEQYENIKVREGVDENSEFKFT | |
| | | LYKNDLLLLKDSENGEQILLRFTSRNDTSKHYVELKPYNRQKFEGSEYLIKS | |
| | | LGTVAKGGQCIKGLGKSNISIYKVRTDVLGNQHIIKNEGDKPKLDF | |
| St1Cas9- | Streptococcus | MSDLVLGLDIGIGSVGVGILNKVTGEIIHKNSRIFPAAQAENNLVRRTNRQG | N622A |
| TH1477 | thermophilus | RRLARRKKHRRVRLNRLFEESGLITDFTKISINLNPYQLRVKGLTDELSNEE | |
| | | LFIALKNMVKHRGISYLDDASDDGNSSVGDYAQIVKENSKQLETKTPGQIQ | |
| | | LERYQTYGQLRGDFTVEKDGKKHRLINVFPTSAYRSEALRILQTQQEFNPQI | |
| | | TDEFINRYLEILTGKRKYYHGPGNEKSRTDYGRYRTSGETLDNIFGILIGKC | |
| | | TFYPDEFRAAKASYTAQEFNLLNDLNNLTVPTETKKLSKEQKNQIINYVKN | |
| | | EKAMGPAKLFKYIAKLLSCDVADIKGYRIDKSGKAEIHTFEAYRKMKTLET | |
| | | LDIEQMDRETLDKLAYVLTLNTEREGIQEALEHEFADGSFSQKQVDELVQF | |
| | | RKANSSIFGKGWHNFSVKLMMELIPELYETSEEQMTILTRLGKQKTTSSSN | |
| | | KTKYIDEKLLTEEIYNPVVAKSVRQAIKIVNAAIKEYGDFDNIVIEMARETN | |
| | | EDDEKKAIQKIQKANKDEKDAAMLKAANQYNGKAELPHSVFHGHKQLAT | |
| | | KIRLWHQQGERCLYTGKTISIHDLINNSNQFEVDHILPLSITFDDSLANKVLV | |
| | | YATANQEKGQRTPYQALDSMDDAWSFRELKAFVRESKTLSNKKKEYLLT | |
| | | EEDISKFDVRKKFIERNLVDTRYASRVVLNALQEHFRAHKIDTKVSVVRGQ | |
| | | FTSQLRRHWGIEKTRDTYHHHAVDALIIAASSQLNLWKKQKNTLVSYSED | |
| | | QLLDIETGELISDDEYKESVFKAPYQHFVDTLKSKEFEDSILFSYQVDSKFN | |
| | | RKISDATIYATRQAKVGKDKADETYVLGKIKDIYTQDGYDAFMKIYKKDK | |
| | | SKFLMYRHDPQTFEKVIEPILENYPNKQINEKGKEVPCNPFLKYKEEHGYIR | |
| | | KYSKKGNGPEIKSLKYYDSKLGNHIDITPKDSNNKVVLQSLKPWRTDVYF | |
| | | NKNTGKYEILGLKYSDMQFEKGTGKYSISKEQYENIKVREGVDENSEFKFT | |
| | | LYKNDLLLLKDSENGEQILLRFTSRNDTSKHYVELKPYNRQKFEGSEYLIKS | |
| | | LGTVVKGGRCIKGLGKSNISIYKVRTDVLGNQHIIKNEGDKPKLDF | |

In some embodiments, the DNA binding domain comprises a Cas domain, e.g., a Cas9 domain. In embodiments, the DNA binding domain comprises a nuclease-active Cas domain, a Cas nickase (nCas) domain, or a nuclease-inactive Cas (dCas) domain. In embodiments, the DNA binding domain comprises a nuclease-active Cas9 domain, a Cas9 nickase (nCas9) domain, or a nuclease-inactive Cas9 (dCas9) domain. In some embodiments, the DNA binding domain comprises a Cas9 domain of Cas9 (e.g., dCas9 and nCas9), Cas12a/Cpf1, Cas12b/C2cl, Cas12c/C2c3, Cas12d/CasY, Cas12e/CasX, Cas12g, Cas12h, or Cas12i. In some embodiments, the DNA binding domain comprises a Cas9 (e.g., dCas9 and nCas9), Cas12a/Cpf1, Cas12b/C2cl, Cas12c/C2c3, Cas12d/CasY, Cas12e/CasX, Cas12g, Cas12h, or Cas12i. In some embodiments, the DNA binding domain comprises an S. pyogenes or an S. thermophilus Cas9, or a functional fragment thereof. In some embodiments, the DNA binding domain comprises a Cas9 sequence, e.g., as described in Chylinski, Rhun, and Charpentier (2013) RNA Biology 10:5, 726-737; incorporated herein by reference. In some embodiments, the DNA binding domain comprises the HNH nuclease subdomain and/or the RuvC1 subdomain of a Cas, e.g., Cas9, e.g., as described herein, or a variant thereof. In some embodiments, the DNA binding domain comprises Cas12a/Cpf1, Cas12b/C2cl, Cas12c/C2c3, Cas12d/CasY, Cas12e/CasX, Cas12g, Cas12h, or Cas12i. In some embodiments, the DNA binding domain comprises a Cas polypeptide (e.g., enzyme), or a functional fragment thereof. In embodiments, the Cas polypeptide (e.g., enzyme) is selected from Cas1, Cas1B, Cas2, Cas3, Cas4, Cas5, Cas5d, Cas5t, Cas5h, Cas5a, Cas6, Cas7, Cas8, Cas8a, Cas8b, Cas8c, Cas9 (e.g., Csn1 or Csx12), Cas10, Cas10d, Cas12a/Cpf1, Cas12b/C2cl, Cas12c/C2c3, Cas12d/CasY, Cas12e/CasX, Cas12g, Cas12h, Cas12i, Csy1, Csy2, Csy3, Csy4, Cse1, Cse2, Cse3, Cse4, Cse5e, Csc1, Csc2, Csa5, Csn1, Csn2, Csm1, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx1S, Csx11, Csf1, Csf2, CsO, Csf4, Csd1, Csd2, Cst1, Cst2, Csh1, Csh2, Csa1, Csa2, Csa3, Csa4, Csa5, Type II Cas effector proteins, Type V Cas effector proteins, Type VI Cas effector proteins, CARF, DinG, Cpf1, Cas12b/C2cl, Cas12c/C2c3, Cas12b/C2cl, Cas12c/C2c3, SpCas9(K855A), eSpCas9(1.1), SpCas9-HF1, hyper accurate Cas9 variant (HypaCas9), homologues thereof, modified or engineered versions thereof, and/or functional fragments thereof. In embodiments, the Cas9 comprises one or more substitutions, e.g., selected from H840A, D10A, P475A, W476A, N477A, D 1125A, W 1126A, and D 1127A. In embodiments, the Cas9 comprises one or more mutations at positions selected from: D10, G12, G17, E762, H840, N854, N863, H982, H983, A984, D986, and/or A987, e.g., one or more substitutions selected from D10A, G12A, G17A, E762A, H840A, N854A, N863A, H982A, H983A, A984A, and/or D986A. In some embodiments, the DNA binding domain comprises a Cas (e.g., Cas9) sequence from Corynebacterium ulcerans, Corynebacterium diphtheria, Spiroplasma syrphidicola, Prevotella intermedia, Spiroplasma taiwanense, Streptococcus iniae, Belliella baltica, Psychroflexus torquis, Streptococcus thermophilus, Listeria innocua, Campylobacter jejuni, Neisseria meningitidis, Streptococcus pyogenes, or Staphylococcus aureus, or a fragment or variant thereof.

In some embodiments, the DNA binding domain comprises a Cpf1 domain, e.g., comprising one or more substitutions, e.g., at position D917, E1006A, D1255 or any combination thereof, e.g., selected from D917A, E1006A, D1255A, D917A/E1006A, D917A/D1255A, E1006A/D1255A, and D917A/E1006A/D1255A.

In some embodiments, the DNA binding domain comprises spCas9, spCas9-VRQR, spCas9-VRER, xCas9 (sp), saCas9, saCas9-KKH, spCas9-MQKSER, spCas9-LRKIQK, or spCas9-LRVSQL.

In some embodiments, the DNA-binding domain comprises an amino acid sequence as listed in Table 5 below, or an amino acid sequence having at least 80%, 85%, 90%, 95%, 96%, 97%, 98%, or 99% sequence identity thereto. In some embodiments, the DNA-binding domain comprises an amino acid sequence that has no more than 1, 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, or 50 differences (e.g., mutations) relative to any of the amino acid sequences described herein.

TABLE 5
Each of the Reference Sequences are incorporated by reference in their entirety.

| Name | Amino Acid Sequence or Reference Sequence |
|---|---|
| Streptococcus pyogenes Cas9 | |
| Exemplary Linker | SGSETPGTSESATPES |
| Exemplary Linker Motif | (SGGS)ₙ |
| Exemplary Linker Motif | (GGGS)ₙ |
| Exemplary Linker Motif | (GGGGS)ₙ |
| Exemplary Linker Motif | (G)ₙ |
| Exemplary Linker Motif | (EAAAK)ₙ |
| Exemplary Linker Motif | (GGS)ₙ |
| Exemplary Linker Motif | (XP)ₙ |
| Cas9 from Streptococcus | NCBI Reference Sequence: NC_002737.2 and Uniprot Reference |
| pyogenes | Sequence: Q99ZW2 |
| Cas9 from Corynebacterium | NCBI Refs: NC_015683.1, NC_017317.1 |
| ulcerans | |
| Cas9 from Corynebacterium | NCBI Refs: NC_016782.1, NC_016786.1 |
| diphtheria | |
| Cas9 from Spiroplasma | NCBI Ref: NC_021284.1 |
| syrphidicola | |
| Cas9 from Prevotella | NCBI Ref: NC_017861.1 |
| intermedia | |
| Cas9 from Spiroplasma | NCBI Ref: NC_021846.1 |
| taiwanense | |
| Cas9 from Streptococcus iniae | NCBI Ref: NC_021314.1 |
| Cas9 from Belliella baltica | NCBI Ref: NC_018010.1 |
| Cas9 from Psychroflexus | NCBI Ref: NC_018721.1 |
| torquisI | |
| Cas9 from Streptococcus | NCBI Ref: YP_820832.1 |
| thermophilus | |
| Cas9 from Listeria innocua | NCBI Ref: NP_472073.1 |
| Cas9 from Campylobacter | NCBI Ref: YP_002344900.1 |
| jejuni | |
| Cas9 from Neisseria | NCBI Ref: YP_002342100.1 |
| meningitidis | |
| dCas9 (D10A and H840A) | |
| Catalytically inactive Cas9 | |
| (dCas9) | |
| Cas9 nickase (nCas9) | |
| Catalytically active Cas9 | |
| CasY | ((ncbi.nlm.nih.gov/protein/APG80656.1) |
| | >APG80656.1 CRISPR-associated protein CasY [uncultured |
| | Parcubacteria group bacterium]) |
| CasX | uniprot.org/uniprot/F0NN87; uniprot.org/uniprot/F0NH53 |
| CasX | >tr\|F0NH53\|F0NH53_SULIR CRISPR associated protein, |
| | Casx OS = Sulfolobus islandicus (strain REY15A) |
| | GN = SiRe_0771 PE = 4 SV = 1 |
| Deltaproteobacteria CasX | |
| Cas12b/C2c1 | ((uniprot.org/uniprot/T0D7A2#2) sp\|T0D7A2\|C2C1_ALIAG |
| | CRISPR-associated endonuclease C2c1 OS = Alicyclobacillus |
| | acidoterrestris (strain ATCC 49025/DSM 3922/CIP 106132/ |
| | NCIMB 13137/GD3B) GN = c2c1 PE = 1 SV = 1) |
| BhCas12b (Bacillus hisashii) | NCBI Reference Sequence: WP_095142515 |
| BvCas12b (Bacillus sp. V3-13) | NCBI Reference Sequence: WP_101661451.1 |
| Wild-type Francisella novicida | |
| Cpf1 | |
| Francisella novicida Cpf1 | |
| D917A | |
| Francisella novicida Cpf1 | |
| E1006A | |
| Francisella novicida Cpf1 | |
| D1255A | |
| Francisella novicida Cpf1 | |
| D917A/E1006A | |
| Francisella novicida Cpf1 | |
| D917A/D1255A | |
| Francisella novicida Cpf1 | |
| E1006A/D1255A | |
| Francisella novicida Cpf1 | |
| D917A/E1006A | |
| SaCas9 | |
| SaCas9n | |
| PAM-binding SpCas9 | |
| PAM-binding SpCas9n | |
| PAM-binding SpEQR Cas9 | |
| PAM-binding SpVQR Cas9 | |
| PAM-binding SpVRER Cas9 | |
| PAM-binding SpVRQR Cas9 | |
| SpyMacCas9 | |

In some embodiments, the Cas polypeptide binds a gRNA that directs DNA binding. In some embodiments, the gRNA comprises, e.g., from 5′ to 3′ (1) a gRNA spacer; (2) a gRNA scaffold. In some embodiments:
(1) Is a Cas9 spacer of ˜18-22 nt, e.g., is 20 nt
(2) Is a gRNA scaffold comprising one or more hairpin loops, e.g., 1, 2, of 3 loops for associating the template with a nickase Cas9 domain. In some embodiments, the gRNA scaffold carries the sequence, from 5′ to 3′,

GTTTTAGAGCTAGAAATAGCAAGTTAAAATAAGGCTAGTCCGTT
ATCAACTTGAAAAAGTGGGACCGAGTCGGTCC.

In some embodiments, a Gene Writing system described herein is used to make an edit in HEK293, K562, U2OS, or HeLa cells. In some embodiment, a Gene Writing system is used to make an edit in primary cells, e.g., primary cortical neurons from E18.5 mice.

In some embodiments, a system or method described herein involves a CRISPR DNA targeting enzyme or system described in US Pat. App. Pub. No. 20200063126, 20190002889, or 20190002875 (each of which is incorporated by reference herein in its entirety) or a functional fragment or variant thereof. For instance, in some embodiments, a GeneWriter polypeptide or Cas endonuclease described herein comprises a polypeptide sequence of any of the applications mentioned in this paragraph, and in some embodiments a guide RNA comprises a nucleic acid sequence of any of the applications mentioned in this paragraph.

In some embodiments, the DNA binding domain (e.g., a target binding domain or a template binding domain) comprises a meganuclease domain, or a functional fragment thereof. In some embodiments, the meganuclease domain possesses endonuclease activity, e.g., double-strand cleavage and/or nickase activity. In other embodiments, the meganuclease domain has reduced activity, e.g., lacks endonuclease activity, e.g., the meganuclease is catalytically inactive. In some embodiments, a catalytically inactive meganuclease is used as a DNA binding domain, e.g., as described in Fonfara et al. Nucleic Acids Res 40(2):847-860 (2012), incorporated herein by reference in its entirety. In embodiments, the DNA binding domain comprises one or more modifications relative to a wild-type DNA binding domain, e.g., a modification via directed evolution, e.g., phage-assisted continuous evolution (PACE).

### Inteins

In some embodiments, as described in more detail below, Intein-N may be fused to the N-terminal portion of a polypeptide (e.g., a Gene Writer polypeptide) described herein, e.g., at a first domain. In embodiments, intein-C may be fused to the C-terminal portion of the polypeptide described herein (e.g., at a second domain), e.g., for the joining of the N-terminal portion to the C-terminal portion, thereby joining the first and second domains. In some embodiments, the first and second domains are each independently chosen from a DNA binding domain and a catalytic domain, e.g., a recombinase domain. In some embodiments, a single domain is split using the intein strategy described herein, e.g., a DNA binding domain, e.g., a dCas9 domain.

In some embodiments, a system or method described herein involves an intein that is a self-splicing protein intron (e.g., peptide), e.g., which ligates flanking N-terminal and C-terminal exteins (e.g., fragments to be joined). An intein may, in some instances, comprise a fragment of a protein that is able to excise itself and join the remaining fragments (the exteins) with a peptide bond in a process known as protein splicing. Inteins are also referred to as “protein inons.” The process of an intein excising itself and joining the remaining portions of the protein is herein termed “protein splicing” or “intein-mediated protein splicing.” In some embodiments, an intein of a precursor protein (an intein containing protein prior to intein-mediated protein splicing) comes from two genes. Such intein is referred to herein as a split intein (e.g., split intein-N and split intein-C). For example, in cyanobacteria, DnaE, the catalytic subunit a of DNA polymerase III, is encoded by two separate genes, dnaE-n and dnaE-c. The intein encoded by the dnaE-n gene may be herein referred as “intein-N.” The intein encoded by the dnaE-c gene may be herein referred as “intein-C.”

Use of inteins for joining heterologous protein fragments is described, for example, in Wood et al., J. Biol. Chem.289(21); 14512-9 (2014) (incorporated herein by reference in its entirety). For example, when fused to separate protein fragments, the inteins IntN and IntC may recognize each other, splice themselves out, and/or simultaneously ligate the flanking N- and C-terminal exteins of the protein fragments to which they were fused, thereby reconstituting a full-length protein from the two protein fragments.

In some embodiments, a synthetic intein based on the dnaE intein, the Cfa-N(e.g., split intein-N) and Cfa-C(e.g., split intein-C) intein pair, is used. Examples of such inteins have been described, e.g., in Stevens et al., J Am Chem Soc. 2016 Feb. 24; 138(7):2162-5 (incorporated herein by reference in its entirety). Non-limiting examples of intein pairs that may be used in accordance with the present disclosure include: Cfa DnaE intein, Ssp GyrB intein, Ssp DnaX intein, Ter DnaE3 intein, Ter ThyX intein, Rma DnaB intein and Cne Prp8 intein (e.g., as described in U.S. Pat. No. 8,394,604, incorporated herein by reference.

In some embodiments, Intein-N and intein-C may be fused to the N-terminal portion of the split Cas9 and the C-terminal portion of a split Cas9, respectively, for the joining of the N-terminal portion of the split Cas9 and the C-terminal portion of the split Cas9. For example, in some embodiments, an intein-N is fused to the C-terminus of the N-terminal portion of the split Cas9, i.e., to form a structure of N [N-terminal portion of the split Cas9]-[intein-N]˜C. In some embodiments, an intein-C is fused to the N-terminus of the C-terminal portion of the split Cas9, i.e., to form a structure of N-[intein-C]˜[C-terminal portion of the split Cas9]-C. The mechanism of intein-mediated protein splicing for joining the proteins the inteins are fused to (e.g., split Cas9) is described in Shah et al., Chem Sci. 2014; 5(1):446-461, incorporated herein by reference. Methods for designing and using inteins are known in the art and described, for example by WO2020051561, WO2014004336, WO2017132580, US20150344549, and US20180127780, each of which is incorporated herein by reference in their entirety.

In some embodiments, a split refers to a division into two or more fragments. In some embodiments, a split Cas9 protein or split Cas9 comprises a Cas9 protein that is provided as an N-terminal fragment and a C-terminal fragment encoded by two separate nucleotide sequences. The polypeptides corresponding to the N-terminal portion and the C-terminal portion of the Cas9 protein may be spliced to form a reconstituted Cas9 protein. In embodiments, the Cas9 protein is divided into two fragments within a disordered region of the protein, e.g., as described in Nishimasu et al., Cell, Volume 156, Issue 5, pp. 935-949, 2014, or as described in Jiang et al. (2016) Science 351: 867-871 and PDB file: 5F9R (each of which is incorporated herein by reference in its entirety). A disordered region may be determined by one or more protein structure determination techniques known in the art, including, without limitation, X-ray crystallography, NMR spectroscopy, electron microscopy (e.g., cryoEM), and/or in silico protein modeling. In some embodiments, the protein is divided into two fragments at any C, T, A, or S, e.g., within a region of SpCas9 between amino acids A292-G364, F445-K483, or E565-T637, or at corresponding positions in any other Cas9, Cas9 variant (e.g., nCas9, dCas9), or other napDNAbp. In some embodiments, protein is divided into two fragments at SpCas9 T310, T313, A456, S469, or C574. In some embodiments, the process of dividing the protein into two fragments is referred to as splitting the protein.

In some embodiments, a protein fragment ranges from about 2-1000 amino acids (e.g., between 2-10, 10-50, 50-100, 100-200, 200-300, 300-400, 400-500, 500-600, 600-700, 700-800, 800-900, or 900-1000 amino acids) in length. In some embodiments, a protein fragment ranges from about 5-500 amino acids (e.g., between 5-10, 10-50, 50-100, 100-200, 200-300, 300-400, or 400-500 amino acids) in length. In some embodiments, a protein fragment ranges from about 20-200 amino acids (e.g., between 20-30, 30-40, 40-50, 50-100, or 100-200 amino acids) in length.

In some embodiments, a portion or fragment of a Gene Writer polypeptide, e.g., as described herein, is fused to an intein. The nuclease can be fused to the N-terminus or the C-terminus of the intein. In some embodiments, a portion or fragment of a fusion protein is fused to an intein and fused to an AAV capsid protein. The intein, nuclease and capsid protein can be fused together in any arrangement (e.g., nuclease-intein-capsid, intein-nuclease-capsid, capsid-intein-nuclease, etc.). In some embodiments, the N-terminus of an intein is fused to the C-terminus of a fusion protein and the C-terminus of the intein is fused to the N-terminus of an AAV capsid protein.

In some embodiments, a Gene Writer polypeptide (e.g., comprising a nickase Cas9 domain) is fused to intein-N and a polypeptide comprising a polymerase domain is fused to an intein-C.

Exemplary nucleotide and amino acid sequences of interns are provided below:

DnaE Intein-N DNA:
TGCCTGTCATACGAAACCGAGATACTGACAGTAGAATATGGCCT
TCTGCCAATCGGGAAGATTGTGGAGAAACGGATAGAATGCACAG
TTTACTCTGTCGATAACAATGGTAACATTTATACTCAGCCAGTT
GCCCAGTGGCACGACCGGGGAGAGCAGGAAGTATTCGAATACTG
TCTGGAGGATGGAAGTCTCATTAGGGCCACTAAGGACCACAAAT
TTATGACAGTCGATGGCCAGATGCTGCCTATAGACGAAATCTTT
GAGCGAGAGTTGGACCTCATGCGAGTTGACAACCTTCCTAAT
DnaE Intein-N Protein:
CLSYETEILTVEYGLLPIGKIVEKRIECTVYSVDNNGNIYTQPV
AQWHDRGEQEVFEYCLEDGSLIRATKDHKFMTVDGQMLPIDEIF
ERELDLMRVDNLPN
DnaE Intein-C DNA:
ATGATCAAGATAGCTACAAGGAAGTATCTTGGCAAACAAAACGT
TTATGATATTGGAGTCGAAAGAGATCACAACTTTGCTCTGAAGA
ACGGATTCATAGCTTCTAAT
Intein-C:
MIKIATRKYLGKQNVYDIGVERDHNFALKNGFIASN
Cfa-N DNA:
TGCCTGTCTTATGATACCGAGATACTTACCGTTGAATATGGCTT
CTTGCCTATTGGAAAGATTGTCGAAGAGAGAATTGAATGCACAG
TATATACTGTAGACAAGAATGGTTTCGTTTACACACAGCCCATT
GCTCAATGGCACAATCGCGGCGAACAAGAAGTATTTGAGTACTG
TCTCGAGGATGGAAGCATCATACGAGCAACTAAAGATCATAAAT
TCATGACCACTGACGGGCAGATGTTGCCAATAGATGAGATATTC
GAGCGGGGCTTGGATCTCAAACAAGTGGATGGATTG CCA
Cfa-N Protein:
CLSYDTEILTVEYGFLPIGKIVEERIECTVYTVDKNGFVYTQPI
AQWHNRGEQEVFEYCLEDGSIIRATKDHKFMTTDGQMLPIDEIF
ERGLDLKQVDGLP
Cfa-C DNA:
ATGAAGAGGACTGCCGATGGATCAGAGTTTGAATCTCCCAAGAA
GAAGAGGAAAGTAAAGATAATATCTCGAAAAAGTCTTGGTACCC
AAAATGTCTATGATATTGGAGTGGAGAAAGATCACAACTTCCTT
CTCAAGAACGGTCTCGTAGCCAGCAAC
Cfa-C Protein:
MKRTADGSEFESPKKKRKVKIISRKSLGTQNVYDIGVEKDHNFL
LKNGLVASN

### Genomic Safe Harbor Sites

In some embodiments, a Gene Writer targets a genomic safe harbor site (e.g., directs insertion of a heterologous object sequence into a position having a safe harbor score of at least 3, 4, 5, 6, 7, or 8). In some embodiments the genomic safe harbor site is a Natural Harbor™ site. In some embodiments, a Natural Harbor™ site is derived from the native target of a mobile genetic element, e.g., a recombinase, transposon, or retrovirus. The native targets of mobile elements may serve as ideal locations for genomic integration given their evolutionary selection. In some embodiments the Natural Harbor™ site is ribosomal DNA (rDNA). In some embodiments the Natural Harbor™ site is 5S rDNA, 18S rDNA, 5.8S rDNA, or 28S rDNA. In some embodiments the Natural Harbor™ site is the Mutsu site in 5S rDNA. In some embodiments the Natural Harbor™ site is the R2 site, the R5 site, the R6 site, the R4 site, the R1 site, the R9 site, or the RT site in 28S rDNA. In some embodiments the Natural Harbor™ site is the R8 site or the R7 site in 18S rDNA. In some embodiments the Natural Harbor™ site is DNA encoding transfer RNA (tRNA). In some embodiments the Natural Harbor™ site is DNA encoding tRNA-Asp or tRNA-Glu. In some embodiments the Natural Harbor™ site is DNA encoding spliceosomal RNA. In some embodiments the Natural Harbor™ site is DNA encoding small nuclear RNA (snRNA) such as U2 snRNA.

Thus, in some aspects, the present disclosure provides a method comprising comprises using a GeneWriter system described herein to insert a heterologous object sequence into a Natural Harbor™ site. In some embodiments, the Natural Harbor™ site is a site described in Table 6 below. In some embodiments, the heterologous object sequence is inserted within 20, 50, 100, 150, 200, 250, 500, or 1000 base pairs of the Natural Harbor™ site. In some embodiments, the heterologous object sequence is inserted within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of the Natural Harbor™ site. In some embodiments, the heterologous object sequence is inserted into a site having at least 70%, 75%, 80%, 85%, 90%, 95%, 96%, 97%, 98%, or 99% identity to a sequence shown in Table 6. In some embodiments, the heterologous object sequence is inserted within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of a site having at least 70%, 75%, 80%, 85%, 90%, 95%, 96%, 97%, 98%, or 99% identity to a sequence shown in Table 6. In some embodiments, the heterologous object sequence is inserted within a gene indicated in Column 5 of Table 6, or within 20, 50, 100, 150, 200, 250, 500, or 1000 base pairs, or within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb, of the gene.

TABLE 6
Natural Harbor™ sites. Column 1 indicates a retrotransposon
that inserts into the Natural Harbor™ site. Column 2 indicates
the gene at the Natural Harbor™ site. Columns 3 and 4 show
exemplary human genome sequence 5′ and 3′ of the insertion
site (for example, 250 bp). Columns 5 and 6 list the example
gene symbol and corresponding Gene ID.

| | | | | Example | |
|---|---|---|---|---|---|
| Target | Target | 5′ flanking | 3′ flanking | Gene | Example |
| Site | Gene | sequence | sequence | Symbol | Gene ID |
| R2 | 28S | CCGGTCCCCCCCGCC | GTAGCCAAATGCCTC | RNA28SN1 | 106632264 |
| | rDNA | GGGTCCGCCCCCGGG | GTCATCTAATTAGTG | | |
| | | GCCGCGGTTCCGCGC | ACGCGCATGAATGGA | | |
| | | GGCGCCTCGCCTCGG | TGAACGAGATTCCCA | | |
| | | CCGGCGCCTAGCAGC | CTGTCCCTACCTACTA | | |
| | | CGACTTAGAACTGGT | TCCAGCGAAACCACA | | |
| | | GCGGACCAGGGGAAT | GCCAAGGGAACGGGC | | |
| | | CCGACTGTTTAATTA | TTGGCGGAATCAGCG | | |
| | | AAACAAAGCATCGCG | GGGAAAGAAGACCCT | | |
| | | AAGGCCCGCGGCGGG | GTTGAGCTTGACTCT | | |
| | | TGTTGACGCGATGTG | AGTCTGGCACGGTGA | | |
| | | ATTTCTGCCCAGTGCT | AGAGACATGAGAGGT | | |
| | | CTGAATGTCAAAGTG | GTAGAATAAGTGGGA | | |
| | | AAGAAATTCAATGAA | GGCCCCCGGCGCCCC | | |
| | | GCGCGGGTAAACGGC | CCCGGTGTCCCCGCG | | |
| | | GGGAGTAACTATGAC | AGGGGCCCGGGGCGG | | |
| | | TCTCTTAAG (SEQ ID | GGTCCGCCG (SEQ ID | | |
| | | NO: 3453) | NO: 3464) | | |
| R4 | 28S | GCGGTTCCGCGCGGC | CGCATGAATGGATGA | RNA28SN1 | 106632264 |
| | rDNA | GCCTCGCCTCGGCCG | ACGAGATTCCCACTG | | |
| | | GCGCCTAGCAGCCGA | TCCCTACCTACTATCC | | |
| | | CTTAGAACTGGTGCG | AGCGAAACCACAGCC | | |
| | | GACCAGGGGAATCCG | AAGGGAACGGGCTTG | | |
| | | ACTGTTTAATTAAAA | GCGGAATCAGCGGGG | | |
| | | CAAAGCATCGCGAAG | AAAGAAGACCCTGTT | | |
| | | GCCCGCGGCGGGTGT | GAGCTTGACTCTAGT | | |
| | | TGACGCGATGTGATT | CTGGCACGGTGAAGA | | |
| | | TCTGCCCAGTGCTCTG | GACATGAGAGGTGTA | | |
| | | AATGTCAAAGTGAAG | GAATAAGTGGGAGGC | | |
| | | AAATTCAATGAAGCG | CCCCGGCGCCCCCCC | | |
| | | CGGGTAAACGGCGGG | GGTGTCCCCGCGAGG | | |
| | | AGTAACTATGACTCT | GGCCCGGGGCGGGGT | | |
| | | CTTAAGGTAGCCAAA | CCGCCGGCCCTGCGG | | |
| | | TGCCTCGTCATCTAAT | GCCGCCGGTGAAATA | | |
| | | TAGTGACG (SEQ ID | CCACTACTC (SEQ ID | | |
| | | NO: 3454) | NO: 3465) | | |
| R5 | 28S | TCCCCCCCGCCGGGT | CCAAATGCCTCGTCA | RNA28SN1 | 106632264 |
| | rDNA | CCGCCCCCGGGGCCG | TCTAATTAGTGACGC | | |
| | | CGGTTCCGCGCGGCG | GCATGAATGGATGAA | | |
| | | CCTCGCCTCGGCCGG | CGAGATTCCCACTGT | | |
| | | CGCCTAGCAGCCGAC | CCCTACCTACTATCCA | | |
| | | TTAGAACTGGTGCGG | GCGAAACCACAGCCA | | |
| | | ACCAGGGGAATCCGA | AGGGAACGGGCTTGG | | |
| | | CTGTTTAATTAAAAC | CGGAATCAGCGGGGA | | |
| | | AAAGCATCGCGAAGG | AAGAAGACCCTGTTG | | |
| | | CCCGCGGCGGGTGTT | AGCTTGACTCTAGTCT | | |
| | | GACGCGATGTGATTT | GGCACGGTGAAGAGA | | |
| | | CTGCCCAGTGCTCTG | CATGAGAGGTGTAGA | | |
| | | AATGTCAAAGTGAAG | ATAAGTGGGAGGCCC | | |
| | | AAATTCAATGAAGCG | CCGGCGCCCCCCCGG | | |
| | | CGGGTAAACGGCGGG | TGTCCCCGCGAGGGG | | |
| | | AGTAACTATGACTCT | CCCGGGGCGGGGTCC | | |
| | | CTTAAGGTAG (SEQ ID | GCCGGCCC (SEQ ID | | |
| | | NO: 3455) | NO: 3466) | | |
| R9 | 28S | CGGCGCGCTCGCCGG | TAGCTGGTTCCCTCCG | RNA28SN1 | 106632264 |
| | rDNA | CCGAGGTGGGATCCC | AAGTTTCCCTCAGGA | | |
| | | GAGGCCTCTCCAGTC | TAGCTGGCGCTCTCG | | |
| | | CGCCGAGGGCGCACC | CAGACCCGACGCACC | | |
| | | ACCGGCCCGTCTCGC | CCCGCCACGCAGTTT | | |
| | | CCGCCGCGCCGGGGA | TATCCGGTAAAGCGA | | |
| | | GGTGGAGCACGAGCG | ATGATTAGAGGTCTT | | |
| | | CACGTGTTAGGACCC | GGGGCCGAAACGATC | | |
| | | GAAAGATGGTGAACT | TCAACCTATTCTCAA | | |
| | | ATGCCTGGGCAGGGC | ACTTTAAATGGGTAA | | |
| | | GAAGCCAGAGGAAAC | GAAGCCCGGCTCGCT | | |
| | | TCTGGTGGAGGTCCG | GGCGTGGAGCCGGGC | | |
| | | TAGCGGTCCTGACGT | GTGGAATGCGAGTGC | | |
| | | GCAAATCGGTCGTCC | CTAGTGGGCCACTTTT | | |
| | | GACCTGGGTATAGGG | GGTAAGCAGAACTGG | | |
| | | GCGAAAGACTAATCG | CGCTGCGGGATGAAC | | |
| | | AACCATCTAG (SEQ ID | CGAACGCC (SEQ ID | | |
| | | NO: 3456) | NO: 3467) | | |
| R8 | 18S | GCATTCGTATTGCGC | TGAAACTTAAAGGAA | RNA18SN1 | 106631781 |
| | rDNA | CGCTAGAGGTGAAAT | TTGACGGAAGGGCAC | | |
| | | TCTTGGACCGGCGCA | CACCAGGAGTGGAGC | | |
| | | AGACGGACCAGAGCG | CTGCGGCTTAATTTG | | |
| | | AAAGCATTTGCCAAG | ACTCAACACGGGAAA | | |
| | | AATGTTTTCATTAATC | CCTCACCCGGCCCGG | | |
| | | AAGAACGAAAGTCGG | ACACGGACAGGATTG | | |
| | | AGGTTCGAAGACGAT | ACAGATTGATAGCTC | | |
| | | CAGATACCGTCGTAG | TTTCTCGATTCCGTGG | | |
| | | TTCCGACCATAAACG | GTGGTGGTGCATGGC | | |
| | | ATGCCGACCGGCGAT | CGTTCTTAGTTGGTGG | | |
| | | GCGGCGGCGTTATTC | AGCGATTTGTCTGGTT | | |
| | | CCATGACCCGCCGGG | AATTCCGATAACGAA | | |
| | | CAGCTTCCGGGAAAC | CGAGACTCTGGCATG | | |
| | | CAAAGTCTTTGGGTT | CTAACTAGTTACGCG | | |
| | | CCGGGGGGAGTATGG | ACCCCCGAGCGGTCG | | |
| | | TTGCAAAGC (SEQ ID | GCGTCCC (SEQ ID NO: | | |
| | | NO: 3457) | 3468) | | |
| R4- | tRNA- | | | TRD- | 100189207 |
| 2_SRa | Asp | | | GTC1-1 | |
| LIN25_ | tRNA- | | | TRE- | 100189384 |
| SM | Glu | | | CTC1-1 | |
| R1 | 28S | TAGCAGCCGACTTAG | ACCTACTATCCAGCG | RNA28SN1 | 106632264 |
| | rDNA | AACTGGTGCGGACCA | AAACCACAGCCAAGG | | |
| | | GGGGAATCCGACTGT | GAACGGGCTTGGCGG | | |
| | | TTAATTAAAACAAAG | AATCAGCGGGGAAAG | | |
| | | CATCGCGAAGGCCCG | AAGACCCTGTTGAGC | | |
| | | CGGCGGGTGTTGACG | TTGACTCTAGTCTGGC | | |
| | | CGATGTGATTTCTGCC | ACGGTGAAGAGACAT | | |
| | | CAGTGCTCTGAATGT | GAGAGGTGTAGAATA | | |
| | | CAAAGTGAAGAAATT | AGTGGGAGGCCCCCG | | |
| | | CAATGAAGCGCGGGT | GCGCCCCCCCGGTGT | | |
| | | AAACGGCGGGAGTAA | CCCCGCGAGGGGCCC | | |
| | | CTATGACTCTCTTAAG | GGGGCGGGGTCCGCC | | |
| | | GTAGCCAAATGCCTC | GGCCCTGCGGGCCGC | | |
| | | GTCATCTAATTAGTG | CGGTGAAATACCACT | | |
| | | ACGCGCATGAATGGA | ACTCTGATCGTTTTTT | | |
| | | TGAACGAGATTCCCA | CACTGACCCGGTGAG | | |
| | | CTGTCCCT (SEQ ID | GCGGGGGG (SEQ ID | | |
| | | NO: 3458) | NO: 3469) | | |
| R6 | 28S | CCCCCCGCCGGGTCC | AAATGCCTCGTCATC | RNA28SN1 | 106632264 |
| | rDNA | GCCCCCGGGGCCGCG | TAATTAGTGACGCGC | | |
| | | GTTCCGCGCGGCGCC | ATGAATGGATGAACG | | |
| | | TCGCCTCGGCCGGCG | AGATTCCCACTGTCC | | |
| | | CCTAGCAGCCGACTT | CTACCTACTATCCAG | | |
| | | AGAACTGGTGCGGAC | CGAAACCACAGCCAA | | |
| | | CAGGGGAATCCGACT | GGGAACGGGCTTGGC | | |
| | | GTTTAATTAAAACAA | GGAATCAGCGGGGAA | | |
| | | AGCATCGCGAAGGCC | AGAAGACCCTGTTGA | | |
| | | CGCGGCGGGTGTTGA | GCTTGACTCTAGTCTG | | |
| | | CGCGATGTGATTTCT | GCACGGTGAAGAGAC | | |
| | | GCCCAGTGCTCTGAA | ATGAGAGGTGTAGAA | | |
| | | TGTCAAAGTGAAGAA | TAAGTGGGAGGCCCC | | |
| | | ATTCAATGAAGCGCG | CGGCGCCCCCCCGGT | | |
| | | GGTAAACGGCGGGAG | GTCCCCGCGAGGGGC | | |
| | | TAACTATGACTCTCTT | CCGGGGCGGGGTCCG | | |
| | | AAGGTAGCC (SEQ ID | CCGGCCCTG (SEQ ID | | |
| | | NO: 3459) | NO: 3470) | | |
| R7 | 18S | GCGCAAGACGGACCA | GGAGCCTGCGGCTTA | RNA18SN1 | 106631781 |
| | rDNA | GAGCGAAAGCATTTG | ATTTGACTCAACACG | | |
| | | CCAAGAATGTTTTCA | GGAAACCTCACCCGG | | |
| | | TTAATCAAGAACGAA | CCCGGACACGGACAG | | |
| | | AGTCGGAGGTTCGAA | GATTGACAGATTGAT | | |
| | | GACGATCAGATACCG | AGCTCTTTCTCGATTC | | |
| | | TCGTAGTTCCGACCA | CGTGGGTGGTGGTGC | | |
| | | TAAACGATGCCGACC | ATGGCCGTTCTTAGTT | | |
| | | GGCGATGCGGCGGCG | GGTGGAGCGATTTGT | | |
| | | TTATTCCCATGACCCG | CTGGTTAATTCCGAT | | |
| | | CCGGGCAGCTTCCGG | AACGAACGAGACTCT | | |
| | | GAAACCAAAGTCTTT | GGCATGCTAACTAGT | | |
| | | GGGTTCCGGGGGGAG | TACGCGACCCCCGAG | | |
| | | TATGGTTGCAAAGCT | CGGTCGGCGTCCCCC | | |
| | | GAAACTTAAAGGAAT | AACTTCTTAGAGGGA | | |
| | | TGACGGAAGGGCACC | CAAGTGGCGTTCAGC | | |
| | | ACCAGGAGT (SEQ ID | CACCCGAG (SEQ ID | | |
| | | NO: 3460) | NO: 3471) | | |
| RT | 28S | GGCCGGGCGCGACCC | AACTGGCTTGTGGCG | RNA28SN1 | 106632264 |
| | rDNA | GCTCCGGGGACAGTG | GCCAAGCGTTCATAG | | |
| | | CCAGGTGGGGAGTTT | CGACGTCGCTTTTTGA | | |
| | | GACTGGGGCGGTACA | TCCTTCGATGTCGGCT | | |
| | | CCTGTCAAACGGTAA | CTTCCTATCATTGTGA | | |
| | | CGCAGGTGTCCTAAG | AGCAGAATTCACCAA | | |
| | | GCGAGCTCAGGGAGG | GCGTTGGATTGTTCA | | |
| | | ACAGAAACCTCCCGT | CCCACTAATAGGGAA | | |
| | | GGAGCAGAAGGGCA | CGTGAGCTGGGTTTA | | |
| | | AAAGCTCGCTTGATC | GACCGTCGTGAGACA | | |
| | | TTGATTTTCAGTACGA | GGTTAGTTTTACCCTA | | |
| | | ATACAGACCGTGAAA | CTGATGATGTGTTGTT | | |
| | | GCGGGGCCTCACGAT | GCCATGGTAATCCTG | | |
| | | CCTTCTGACCTTTTGG | CTCAGTACGAGAGGA | | |
| | | GTTTTAAGCAGGAGG | ACCGCAGGTTCAGAC | | |
| | | TGTCAGAAAAGTTAC | ATTTGGTGTATGTGCT | | |
| | | CACAGGGAT (SEQ ID | TGGC (SEQ ID NO: | | |
| | | NO: 3461) | 3472) | | |
| Mutsu | 5S rDNA | GTCTACGGCCATACC | TGAACGCGCCCGATC | RNA5S1 | 100169751 |
| | | ACCC (SEQ ID NO: | TCGTCTGATCTCGGA | | |
| | | 3462) | AGCTAAGCAGGGTCG | | |
| | | | GGCCTGGTTAGTACT | | |
| | | | TGGATGGGAGACCGC | | |
| | | | CTGGGAATACCGGGT | | |
| | | | GCTGTAGGCTTT (SEQ | | |
| | | | ID NO: 3473) | | |
| Utopia/ | U2 | ATCGCTTCTCGGCCTT | TCTGTTCTTATCAGTT | RNU2-1 | 6066 |
| Keno | snRNA | TTGGCTAAGATCAAG | TAATATCTGATACGT | | |
| | | TGTAGTA (SEQ ID NO: | CCTCTATCCGAGGAC | | |
| | | 3463) | AATATATTAAATGGA | | |
| | | | TTTTTGGAGCAGGGA | | |
| | | | GATGGAATAGGAGCT | | |
| | | | TGCTCCGTCCACTCCA | | |
| | | | CGCATCGACCTGGTA | | |
| | | | TTGCAGTACCTCCAG | | |
| | | | GAACGGTGCACCC | | |
| | | | (SEQ ID NO: 3474) | | |

### Additional Functional Characteristics for Gene Writers™

A Gene Writer as described herein may, in some instances, be characterized by one or more functional measurements or characteristics. In some embodiments, the DNA binding domain (e.g., target binding domain) has one or more of the functional characteristics described below. In some embodiments, the template binding domain has one or more of the functional characteristics described below. In some embodiments, the template (e.g., template DNA) has one or more of the functional characteristics described below. In some embodiments, the target site altered by the Gene Writer has one or more of the functional characteristics described below following alteration by the Gene Writer.

### Gene Writer Polypeptide

### DNA Binding Domain

In some embodiments, the DNA binding domain is capable of binding to a target sequence (e.g., a dsDNA target sequence) with greater affinity than a reference DNA binding domain. In some embodiments, the reference DNA binding domain is a DNA binding domain from phiC31 recombinase from the Streptomyces bacteriophage phiC31. In some embodiments, the DNA binding domain is capable of binding to a target sequence (e.g., a dsDNA target sequence) with an affinity between 100 pM-10 nM (e.g., between 100 pM-1 nM or 1 nM-10 nM).

In some embodiments, the affinity of a DNA binding domain for its target sequence (e.g., dsDNA target sequence) is measured in vitro, e.g., by thermophoresis, e.g., as described in Asmari et al. Methods 146:107-119 (2018) (incorporated by reference herein in its entirety).

In embodiments, the DNA binding domain is capable of binding to its target sequence (e.g., dsDNA target sequence), e.g, with an affinity between 100 pM-10 nM (e.g., between 100 pM-1 nM or 1 nM-10 nM) in the presence of a molar excess of scrambled sequence competitor dsDNA, e.g., of about 100-fold molar excess.

In some embodiments, the DNA binding domain is found associated with its target sequence (e.g., dsDNA target sequence) more frequently than any other sequence in the genome of a target cell, e.g., human target cell, e.g., as measured by ChIP-seq (e.g., in HEK293T cells), e.g., as described in He and Pu (2010) Curr. Protoc Mol Biol Chapter 21 (incorporated herein by reference in its entirety). In some embodiments, the DNA binding domain is found associated with its target sequence (e.g., dsDNA target sequence) at least about 5-fold or 10-fold, more frequently than any other sequence in the genome of a target cell, e.g., as measured by ChIP-seq (e.g., in HEK293T cells), e.g., as described in He and Pu (2010), supra.

### Template Binding Domain

In some embodiments, the template binding domain is capable of binding to a template DNA with greater affinity than a reference DNA binding domain. In some embodiments, the reference DNA binding domain is a DNA binding domain from phiC31 recombinase from the Streptomyces bacteriophage phiC31. In some embodiments, the template binding domain is capable of binding to a template DNA with an affinity between 100 pM-10 nM (e.g., between 100 pM-1 nM or 1 nM-10 nM). In some embodiments, the affinity of a DNA binding domain for its template DNA is measured in vitro, e.g., by thermophoresis, e.g., as described in Asmari et al. Methods 146:107-119 (2018) (incorporated by reference herein in its entirety). In some embodiments, the affinity of a DNA binding domain for its template DNA is measured in cells (e.g., by FRET or ChIP-Seq).

In some embodiments, the DNA binding domain is associated with the template DNA in vitro with at least 50% template DNA bound in the presence of 10 nM competitor DNA, e.g., as described in Yant et al. Mol Cell Biol 24(20):9239-9247 (2004) (incorporated by reference herein in its entirety). In some embodiments, the DNA binding domain is associated with the template DNA in cells (e.g., in HEK293T cells) at a frequency at least about 5-fold or 10-fold higher than with a scrambled DNA. In some embodiments, the frequency of association between the DNA binding domain and the template DNA or scrambled DNA is measured by ChIP-seq, e.g., as described in He and Pu (2010), supra.

### Target Site

In some embodiments, after Gene Writing, the target site surrounding the integrated sequence contains a limited number of insertions or deletions, for example, in less than about 50% or 10% of integration events, e.g., as determined by long-read amplicon sequencing of the target site, e.g., as described in Karst et al. Nature Methods 18:165-169 (2021) (incorporated by reference herein in its entirety). For example, indels have been observed after the integration of insert DNA into human genome pseudosites by phiC31 integrase, as described in Thyagarajan et al Mol Cell Biol 21(12):3926-3934 (2001), the teachings of which are incorporated herein by reference in its entirety. In some embodiments, a Gene Writing system of this invention may result in a genomic modification (e.g., an insertion or deletion) at the target site (e.g., the site of insert DNA integration, e.g., adjacent to the integration of the insert DNA) comprising less than nt, e.g., less than 20, 19, 18, 17, 16, 15, 14, 13, 12, 11, 10, 9, 8, 7, 6, 5, 4, 3, 2, or less than 1 nt of DNA. In some embodiments, a Gene Writing system of this invention may result in an insertion at the target site (e.g., the site of insert DNA integration, e.g., adjacent to the integration of the insert DNA) comprising less than 20 nucleotides or base pairs, e.g., less than 20, 19, 18, 17, 16, 15, 14, 13, 12, 11, 10, 9, 8, 7, 6, 5, 4, 3, 2, or less than 1 nucleotides or base pairs of DNA. In some embodiments, a Gene Writing system of this invention may result in a deletion at the target site (e.g., the site of insert DNA integration, e.g., adjacent to the integration of the insert DNA) comprising less than 20 nucleotides or base pairs, e.g., less than 20, 19, 18, 17, 16, 15, 14, 13, 12, 11, 10, 9, 8, 7, 6, 5, 4, 3, 2, or less than 1 nucleotide or base pair of genomic DNA. In some embodiments, the fraction of insertion or deletion events is lower when a core region, e.g., a central dinucleotide, of a recognition sequence at a target site, e.g., an attB, attP, or pseudosite thereof, comprises 100% identity to a core region, e.g., a central dinucleotide, of a recognition sequence, e.g., an attP or attB site, on the insert DNA. In some embodiments, the fraction of unintended insertion or deletion events is lower, e.g., at least 1.1, 1.2, 1.3, 1.4, 1.5, 1.6, 1.7, 1.8, 1.9, 2.0, 3.0, 4.0, 5.0, 10, 20, 30, 40, 50, 60, 70, 80, 90, or at least 100-fold lower at targeted genomic sites when the central dinucleotide of the recognition sequence at the target site is identical to the central dinucleotide of the recognition sequence in the insert DNA.

In some embodiments, the target site does not show multiple insertion events, e.g., head-to-tail or head-to-head duplications, e.g., as determined by long-read amplicon sequencing of the target site, e.g., as described in Karst et al. (2021), supra, or by molecular combing (Example 29). In some embodiments, the target site shows less than 100 insert copies at the target site, e.g., 75 insert copies, 50 insert copies, 45 insert copies, 40 insert copies, 35 insert copies, 30 insert copies, 25 insert copies, 20 insert copies, 15 insert copies, 14 insert copies, 13 insert copies, 12 insert copies, 11 insert copies, 10 insert copies, 9 insert copies, 8 insert copies, 7 insert copies, 6 insert copies, 5 insert copies, 4 insert copies, 3 insert copies, 2 insert copies, or a single insert copy. In some embodiments, target sites showing more than one copy of the insert sequence are present in less than 95% of target sites containing inserts, e.g., in less than 90%, 80%, 70%, 60%, 50%, 40%, 30%, 20%1, 0%, 9%, 8%, 7%, 6%, 4%, 4%, 3%, 2% or less than 1% of target sites containing inserts, e.g., as determined by long-read amplicon sequencing of the target site, e.g., as described in Karst et al. (2021), supra, or by molecular combing (Example 29). In some embodiments, target sites showing more than two copies of the insert sequence are present in less than 95% of target sites containing inserts, e.g., in less than 90%, 80%, 70%, 60%, 50%, 40%, 30%, 20%, 10%, 9%, 8%, 7%, 6%, 4%, 4%, 3%, 2% or less than 1% of target sites containing inserts, e.g., as determined by long-read amplicon sequencing of the target site, e.g., as described in Karst et al. (2021), supra, or by molecular combing (Example 29). In some embodiments, target sites showing more than three copies of the insert sequence are present in less than 95% of target sites containing inserts, e.g., in less than 90%, 80%, 70%, 60%, 50%, 40%, 30%, 20%, 10%, 9%, 8%, 7%, 6%, 4%, 4%, 3%, 2% or less than 1% of target sites containing inserts, e.g., as determined by long-read amplicon sequencing of the target site, e.g., as described in Karst et al. (2021), supra, or by molecular combing (Example 29). In some embodiments, the target site shows at least 1, 2, 3, 4, 5, 6, 7, 8, 9, 10 or more copies per target site. In some embodiments, target sites showing multiple copies of the insert sequence are present in 1%, 5%, 10%, 20%, 30%, 40%, 50% 60%, 70%, 80%, 90%, 95%, 99% or more of target sites containing inserts, e.g., as determined by long-read amplicon sequencing of the target site, e.g., as described in Karst et al. (2021), supra, or by molecular combing (Example 29). In some embodiments, the copies are concatemers, i.e., are concatemerized. In some embodiments, the target site contains an integrated sequence corresponding to the template DNA (e.g., an entire plasmid, minicircle, or viral vector genome). In some embodiments, the target site contains a completely integrated template molecule. In some embodiments, the target site contains components of the vector DNA, e.g., AAV ITRs. In some embodiments, the target site contains 0, 1, 2, 3, 4, 5, 6, 7, 8, 9, 10 or more ITRs after integration. In some embodiments, at least one ITR is present in at least 1% of target sites after integration, e.g., at least 1%, 5%, 10%, 15%, 20%, 25%, 50%, 60%, 70%, 80%, 90, 95%, 96%, 97%, 98%, or at least 99% of target sites after integration. In some embodiments, at least one ITR is present in less than 50% of target sites after integration, e.g., less than 50%, 40%, 30%, 20%, 10%, 9%, 8%, 7%, 6%, 4%, 4%, 3%, 2% or less than 1% of target sites after integration, e.g., as determined by long-read amplicon sequencing of the target site, e.g., as described in Karst et al. (2021), supra, or by molecular combing (Example 29). In some embodiments, the multiple copies are arranged in head-to-head, tail-to-tail, or head-to-tail arrangements, or a mixture thereof. In some embodiments, e.g., when a template DNA is first excised from a viral vector or plasmid by a first recombination event prior to integration, the target site does not contain insertions comprising DNA exogenous to the recognition site-flanked cassette, e.g., vector DNA, e.g., AAV ITRs, in more than about 50% of events, e.g., in more than about 50%, 40%, 30%, 20%, 10%, 9%, 8%, 7%, 6%, 4%, 4%, 3%, 2% or more than about 1% of events, e.g., as determined by long-read amplicon sequencing of the target site, e.g., as described in Karst et al. (2021), supra, or by molecular combing (Example 29). In some embodiments, the integrated DNA does not comprise any bacterial antibiotic resistance gene.

In some embodiments, the DNA integrated at a target site by a Gene Writing system described herein comprises terminal hybrid recognition sequences (e.g., a first and/or second parapalindromic sequence, e.g., as described herein), e.g., attL and attR sequences formed by recombination between a recognition site of the insert DNA, e.g., an attP or attB of the insert DNA, and a recognition site in the target DNA, e.g., an attP or attB site or pseudosite thereof. In some embodiments, the integrated DNA comprises one or more ITRs, e.g., 1, 2, 3, 4, or more ITRs, between the terminal hybrid recognition sequences, e.g., attL and attR sequences. In some embodiments, at least 1% of target sites with integrated DNA comprise ITRs between the terminal hybrid recognition sequences, e.g., attL and attR sequences, e.g. at least 1%, 5%, 10%, 20%, 30%, 40%, 50%, 60%, 70%, 80%, or at least 90% of integrated DNA. In some embodiments, the integrated DNA that comprises ITRs between terminal hybrid recognition sequences, e.g., attL and attR sequences, comprises a single copy of insert DNA, e.g., is a monomeric insertion. In some embodiments, a monomeric insertion comprises terminal hybrid recognition sequences, e.g., attL and attR sequences, and lacks any internal ITRs. In some embodiments, a monomeric insertion comprises terminal hybrid recognition sequences, e.g., attL and attR sequences, and a single internal ITR. In some embodiments, a monomeric insertion comprises terminal hybrid recognition sequences, e.g., attL and attR sequences, and multiple internal ITRs, e.g., two internal ITRs. In some embodiments, the integrated DNA that comprises ITRs between terminal hybrid recognition sequences, e.g., attL and attR sequences, comprises multiple copies of insert DNA, e.g., is a concatemeric insertion. In some embodiments, a concatemeric insertion comprises terminal hybrid recognition sequences, e.g., attL and attR sequences, and at least two, e.g., at least 2, 3, or 4 copies of the insert DNA. In some embodiments, insertions comprising terminal hybrid recognition sequences, e.g., attL and attR sequences, that comprise fewer copies of the insert DNA are present at a higher frequency as compared to those with more copies of the insert DNA (e.g., insertions with 1 copy are present at higher frequency than insertions with 2 copies, insertions with 2 copies are present at higher frequency than insertions with 3 copies, or insertions with 1 copy are present at higher frequency than insertions with 3 copies), show a higher frequency of occurrence, e.g., are 1.1, 1.2, 1.3, 1.4, 1.5, 2.0, 2.5, 3.0, 4.0, 5.0, 10, 20, 30, 40, 50, 60, 70, 80, 90, 100, or more times more frequent. In some embodiments, monomeric insertions are present more frequently than dimeric insertions, e.g, are at least 1.1, 1.2, 1.3, 1.4, 1.5, 2.0, 2.5, 3.0, 4.0, 5.0, 10, 20, 30, 40, 50, 60, 70, 80, 90, 100, or more times more frequent than dimeric insertions. In some embodiments, dimeric insertions are present more frequently than trimeric insertions, e.g, are at least 1.1, 1.2, 1.3, 1.4, 1.5, 2.0, 2.5, 3.0, 4.0, 5.0, 10, 20, 30, 40, 50, 60, 70, 80, 90, 100, or more times more frequent than trimeric insertions. In some embodiments, monomeric plus dimeric insertions are present more frequently than concatameric insertions (3 or more insertions), e.g, are at least 1.1, 1.2, 1.3, 1.4, 1.5, 2.0, 2.5, 3.0, 4.0, 5.0, 10, 20, 30, 40, 50, 60, 70, 80, 90, 100, or more times more frequent than concatameric insertions. In some embodiments, a concatemeric insertion comprises terminal hybrid recognition sequences, e.g., attL and attR sequences, and one or more internal recombinase recognition sequences, e.g., 1, 2, 3, 4, or more internal recognition sequences, e.g., attB or attP sequences. In some embodiments, a concatemeric insertion comprises terminal hybrid recognition sequences, e.g., attL and attR sequences, and one or more internal ITRs, e.g., 1, 2, 3, 4, 5, 6 or more internal ITRs. The copy number of insert DNA, recognition sequences, and ITRs, as well as the relative positioning of these components, as described herein, can be determined using molecular combing as described in Example 29 and in Kaykov et al Sci Rep 6:19636 (2016), incorporated herein by reference in its entirety.

In some embodiments, insertion events may occur in which the integrated DNA does not comprise terminal hybrid recognition sequences, e.g., attL and attR sequences. In some embodiments, integrated DNA may comprise one terminal recognition sequence, e.g., attL or attR sequence. In some embodiments, integrated DNA may not have any terminal hybrid recognition sequences, e.g., attL or attR, e.g., neither terminus of the integrated DNA comprises a hybrid recognition sequence, e.g., attL or attR sequence. In some embodiments, integrated DNA that does not comprise terminal hybrid recognition sequences, e.g., attL or attR sequences, comprises a fragment of an insert DNA (e.g., an incomplete insert DNA, e.g., an insert DNA with an incomplete promoter, gene, or heterologous object sequence). In some embodiments, integrated DNA that does not comprise terminal hybrid recognition sequences, e.g., attL or attR sequences, comprises an incomplete multiple insert DNA sequences, e.g., contains less than 1, more than 1 and less than 2, more than 2 and less than 3, more than 3 and less than 4, or another incomplete multiple number of copies of the complete insert DNA.

In some embodiments, following the use of a Gene Writing system, newly integrated DNA that comprises terminal hybrid recognition sequences, e.g., attL and attR sequences, is present at a higher frequency in a cell or population of cells, e.g., comprises more than 50%, more than 60%, more than 70%, more than 80%, more than 90%, more than 95%, more than 96%, more than 97%, more than 98%, more than 99%, more than 99.5%, or more than 99.9% of total insertion events, compared to newly integrated DNA that comprises one or fewer terminal hybrid recognition sequences, e.g., attL or attR sequences, as measured by an assay described herein, e.g., long-read sequencing or molecular combing. In some embodiments, following the use of a Gene Writing system, newly integrated DNA that comprises terminal hybrid recognition sequences, e.g., attL and attR sequences, comprises a lower average insert DNA copy number per insertion event, e.g., comprises at least 0.1, 0.2, 0.3, 0.4, 0.5, 0.6, 0.7, 0.8, 0.9, 1.0, 1.5, or 2.0 copies fewer per insertion event on average, as compared to the average insert DNA copy number of integration events that comprise one or fewer terminal hybrid recognition sequences, e.g., attL or attP sequences. In some embodiments, following the use of a Gene Writing system, newly integrated DNA that comprises terminal hybrid recognition sequences, e.g., attL and attR sequences, comprises a higher percentage of complete insert DNA sequences, e.g., comprises at least 0.1×, 0.2×, 0.3×, 0.4×, 0.5×, 0.6×, 0.7×, 0.8×, 0.9×, 1.0×, 1.5×, 2.0×, 3×, 4×, 5×, 6×, 7×, 8×, 9×, 10× or more percent complete insert DNA sequences, as compared to the percentage of insert DNA sequences that comprise one or fewer terminal hybrid recognition sequences, e.g., attL or attP sequences.

In some embodiments, a Gene Writer described herein is capable of site-specific editing of target DNA, e.g., insertion of template DNA into a target DNA. In some embodiments, a site-specific Gene Writer is capable of generating an edit, e.g., an insertion, that is present at the target site with a higher frequency than any other site in the genome. In some embodiments, a site-specific Gene Writer is capable of generating an edit, e.g., an insertion in a target site at a frequency of at least 2, 3, 4, 5, 10, 50, 100, or 1000-fold that of the frequency at all other sites in the human genome. In some embodiments, the location of integration sites is determined by unidirectional sequencing, e.g., as in Example 18. The incorporation of unique molecular identifiers (UMI) in the adapters or primers used in library preparation allows the quantification of discrete insertion events, which can be compared between on-target insertions and all other insertions to determine the preference for the defined target site. In some embodiments, an inverse PCR approach is used to determine the integration sites targeted by a particular Gene Writer, e.g., as in Example 30.

In some embodiments, a Gene Writing system is used to edit a target DNA sequence that is present at a single location in the human genome. In some embodiments, a Gene Writing system is used to edit a target DNA sequence that is present at a single location in the human genome on a single homologous chromosome, e.g., is haplotype-specific. In some embodiments, a Gene Writing system is used to edit a target DNA sequence that is present at a single location in the human genome on two homologous chromosomes. In some embodiments, a Gene Writing system is used to edit a target DNA sequence that is present in multiple locations in the genome, e.g., at least 2, 3, 4, 5, 10, 20, 50, 100, 200, 500, 1000, 5000, 10000, 100000, 200000, 500000, 1000000 (e.g., Alu elements) locations in the genome. In some embodiments, a Gene Writing system used herein performs integration at a single target sequence in the human genome, that may be present in one or more locations. In some embodiments, a Gene Writing system used herein performs integration at multiple sequences that are present at least once in the human genome, e.g., recognizes more than 1, e.g., more than 1, 2, 3, 4, 5, 10, 20, 50, or more than 100 sequences, or less than 100, e.g., less than 100, 90, 80, 70, 60, 50, 40, 30, 25, 20, 15, 10, or less than 5 sequences that are present at least once in the human genome. Thus, in some embodiments, a Gene Writer described herein may result in the integration of an insert DNA at at least 1, e.g., at least 1, 2, 3, 4, 5, 6, 7, 8, 9, or at least 10 copies per cell, or less than 10, e.g., less than 10, 9, 8, 7, 6, 5, 4, 3, or less than 2 copies per cell.

In some embodiments, a Gene Writer system is able to edit a genome without introducing undesirable mutations. In some embodiments, a Gene Writer system is able to edit a genome by inserting a template, e.g., template DNA, into the genome. In some embodiments, the resulting modification in the genome contains minimal mutations relative to the template DNA sequence. In some embodiments, the average error rate of genomic insertions relative to the template DNA is less than 10-4, 10-5, or 10-6 mutations per nucleotide. In some embodiments, the number of mutations relative to a template DNA that is introduced into a target cell averages less than 1, 2, 3, 4, 5, 10, 20, 30, 40, 50, 60, 70, 80, 90, or 100 nucleotides per genome. In some embodiments, the error rate of insertions in a target genome is determined by long-read amplicon sequencing across known target sites, e.g., as described in Karst et al. (2021), supra, and comparing to the template DNA sequence. In some embodiments, errors enumerated by this method include nucleotide substitutions relative to the template sequence. In some embodiments, errors enumerated by this method include nucleotide deletions relative to the template sequence. In some embodiments, errors enumerated by this method include nucleotide insertions relative to the template sequence. In some embodiments, errors enumerated by this method include a combination of one or more of nucleotide substitutions, deletions, or insertions relative to the template sequence.

Efficiency of integration events can be used as a measure of editing of target sites or target cells by a Gene Writer system. In some embodiments, a Gene Writer system described herein is capable of integrating a heterologous object sequence in a fraction of target sites or target cells. In some embodiments, a Gene Writer system is capable of editing at least 1%, 2%, 5%, 10%, 20%, 30%, 40%, 50%, 60%, 70%, 80%, 90%, 95%, 96%, 97%, 98%, 99%, 99.5%, 99.9% or 100% of target loci as measured by the detection of the edit when amplifying across the target and analyzing with long-read amplicon sequencing, e.g., as described in Karst et al. (2021), supra. In some embodiments, a Gene Writer system is capable of editing cells at an average copy number of at least 0.1, e.g., at least 0.1, 0.5, 1, 2, 3, 4, 5, 10, or 100 copies per genome as normalized to a reference gene, e.g., RPP30, across a population of cells, e.g., as determined by ddPCR with transgene-specific primer-probe sets, e.g., as according to the methods in Lin et al. Hum Gene Ther Methods 27(5):197-208 (2016).

In some embodiments, the copy number per cell is analyzed by single-cell ddPCR (sc-ddPCR), e.g., as according to the methods of Igarashi et al. Mol Ther Methods C/in Dev 6:8-16 (2017), incorporated herein by reference in its entirety. In some embodiments, at least 1%, e.g., at least 1%, 2%, 5%, 10%, 20%, 30%, 40%, 50%, 60%, 70%, 80%, 90%, 95%, 96%, 97%, 98%, 99%, 99.5%, 99.9% or 100%, of target cells are positive for integration as assessed by sc-ddPCR using transgene-specific primer-probe sets. In some embodiments, the average copy number is at least 0.1, e.g., at least 0.1, 0.5, 1, 2, 3, 4, 5, 10, or 100 copies per cell as measured by sc-ddPCR using transgene-specific primer-probe sets.

In some embodiments, the target site comprises a pair of nucleic acid sequences, wherein one of the nucleic acid sequences is either a palindrome relative to the other nucleic acid sequence, or has at least 20% (e.g., at least 20%, 25%, 30%, 35%, 40%, 45%, 50%, 55%, 60%, 65%, 70%, 75%, 80%, 85%, 90%, 95%, 96%, 97%, 98%, 99%, or 100%), e.g., at least 50%, sequence identity to a palindrome relative to the other nucleic acid sequence, or has no more than 1, 2, 3, 4, 5, 6, 7, 8, 9, 10, 11, 12, 13, 14, 15, 16, 17, 18, 19, or 20 sequence mismatches relative to the other nucleic acid sequence.

### Template DNA

In some embodiments, a template DNA as described herein comprises a nucleic acid sequence that can be integrated into a target DNA molecule, e.g., by a recombinase polypeptide (e.g., a serine recombinase polypeptide), e.g., as described herein. The template DNA typically is able to bind one or more recombinase polypeptides (e.g., a plurality of copies of a recombinase polypeptide) of the system. In some embodiments the template DNA comprises a region that is capable of binding a recombinase polypeptide (e.g., a recognition sequence as described herein). In some embodiments, the template DNA is reverse transcribed from a template RNA, e.g., by a reverse transcriptase polypeptide domain, e.g., as described herein.

An template DNA may, in some embodiments, comprise an object sequence for insertion into a target DNA. The object sequence may be coding or non-coding. In some embodiments, the object sequence may contain an open reading frame. In some embodiments the template DNA comprises a Kozak sequence. In some embodiments the template DNA comprises an internal ribosome entry site. In some embodiments the template DNA comprises a self-cleaving peptide such as a T2A or P2A site. In some embodiments the template DNA comprises a start codon. In some embodiments the template DNA comprises a splice acceptor site. In some embodiments the template DNA comprises a splice donor site. In some embodiments the template DNA comprises a microRNA binding site, e.g., downstream of the stop codon. In some embodiments the template DNA comprises a polyA tail, e.g., downstream of the stop codon of an open reading frame. In some embodiments the template DNA comprises one or more exons. In some embodiments the template DNA comprises one or more introns. In some embodiments the template DNA comprises a eukaryotic transcriptional terminator. In some embodiments the template DNA comprises an enhanced translation element or a translation enhancing element. In some embodiments the template DNA comprises a microRNA sequence, a siRNA sequence, a guide RNA sequence, a piwi RNA sequence. In some embodiments the template DNA comprises a gene expression unit composed of at least one regulatory region operably linked to an effector sequence. The effector sequence may be a sequence that is transcribed into RNA (e.g., a coding sequence or a non-coding sequence such as a sequence encoding a micro RNA). In some embodiments, the object sequence may contain a non-coding sequence. For example, the template DNA may comprise a promoter or enhancer sequence. In some embodiments the template DNA comprises a tissue specific promoter or enhancer, each of which may be unidirectional or bidirectional. In some embodiments the promoter is an RNA polymerase I promoter, RNA polymerase II promoter, or RNA polymerase III promoter. In some embodiments the promoter comprises a TATA element. In some embodiments the promoter comprises a B recognition element. In some embodiments the promoter has one or more binding sites for transcription factors.

In some embodiments the object sequence of the template DNA is inserted into a target genome in an endogenous intron. In some embodiments the object sequence of the template DNA is inserted into a target genome and thereby acts as a new exon. In some embodiments the insertion of the object sequence into the target genome results in replacement of a natural exon or the skipping of a natural exon. In some embodiments the object sequence of the template DNA is inserted into the target genome in a genomic safe harbor site, such as AAVS1, CCR5, or ROSA26. In some embodiment the object sequence of the template DNA is added to the genome in an intergenic or intragenic region. In some embodiments the object sequence of the template DNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous active gene. In some embodiments the object sequence of the template DNA is added to the genome 5′ or 3′ within 0.1 kb, 0.25 kb, 0.5 kb, 0.75, kb, 1 kb, 2 kb, 3 kb, 4 kb, 5 kb, 7.5 kb, 10 kb, 15 kb, 20 kb, 25 kb, 50, 75 kb, or 100 kb of an endogenous promoter or enhancer. In some embodiments the object sequence of the template DNA can be, e.g., 50-50,000 base pairs (e.g., between 50-40,000 bp, between 500-30,000 bp between 500-20,000 bp, between 100-15,000 bp, between 500-10,000 bp, between 50-10,000 bp, between 50-5,000 bp. In some embodiments the object sequence of the template DNA can be, e.g., 1-50 base pairs.

In certain embodiments, an template DNA can be identified, designed, engineered and constructed to contain sequences altering or specifying the genome function of a target cell or target organism, for example by introducing a heterologous coding region into a genome; affecting or causing exon structure/alternative splicing; causing disruption of an endogenous gene; causing transcriptional activation of an endogenous gene; causing epigenetic regulation of an endogenous DNA; causing up- or down-regulation of operably liked genes, etc. In certain embodiments, an template DNA can be engineered to contain sequences coding for exons and/or transgenes, provide for binding sites to transcription factor activators, repressors, enhancers, etc., and combinations of thereof. In other embodiments, the coding sequence can be further customized with splice acceptor sites, poly-A tails.

As an alternative to other methods of delivery described herein, in some embodiments, nucleic acid (e.g., encoding a recombinase, or a template nucleic acid, or both) delivered to cells is designed as minicircles, where plasmid backbone sequences not pertaining to Gene Writing™ are removed before administration to cells. Minicircles have been shown to result in higher transfection efficiencies and gene expression as compared to plasmids with backbones containing bacterial parts (e.g., bacterial origin of replication, antibiotic selection cassette) and have been used to improve the efficiency of transposition (Sharma et al. Mol Ther Nucleic Acids 2:E74 (2013)). In some embodiments, the DNA vector encoding the Gene Writer™ polypeptide is delivered as a minicircle. In some embodiments, the DNA vector containing the Gene Writer™ template is delivered as a minicircle. In some embodiments of such alternative means for delivering a nucleic acid, the bacterial parts are flanked by recombination sites, e.g., attP/attB, loxP, FRT sites. In some embodiments, the addition of a cognate recombinase results in intramolecular recombination and excision of the bacterial parts. In some embodiments, the recombinase sites are recognized by phiC31 recombinase. In some embodiments, the recombinase sites are recognized by Cre recombinase. In some embodiments, the recombinase sites are recognized by FLP recombinase. In some embodiments, minicircles are generated in a bacterial production strain, e.g., an E. coli strain stably expressing inducible minicircle assembling enzymes, e.g., a producer strain as according to Kay et al. Nat Biotechnol 28(12):1287-1289 (2010). Minicircle DNA vector preparations and methods of production are described in U.S. Pat. No. 9,233,174, incorporated herein by reference in its entirety.

In addition to plasmid DNA, minicircles can be generated by excising the desired construct, e.g., recombinase expression cassette or therapeutic expression cassette, from a viral backbone, e.g., an AAV vector. Previously, it has been shown that excision and circularization of the donor sequence from a viral backbone may be important for transposase-mediated integration efficiency (Yant et al. Nat Biotechnol 20(10):999-1005 (2002)). In some embodiments, minicircles are first formulated and then delivered to target cells. In other embodiments, minicircles are formed from a DNA vector (e.g., plasmid DNA, rAAV, scAAV, ceDNA, doggybone DNA) intracellularly by co-delivery of a recombinase, resulting in excision and circularization of the recombinase recognition site-flanked nucleic acid, e.g., a nucleic acid encoding the Gene Writer™ polypeptide, or DNA template, or both. In some embodiments, the same recombinase is used for a first excision event (e.g., intramolecular recombination) and a second integration (e.g., target site integration) event. In some embodiments, the recombination site on an excised circular DNA (e.g., after a first recombination event, e.g., intramolecular recombination) is used as the template recognition site for a second recombination (e.g., target site integration) event.

In some embodiments, minicircle DNA as described herein is generated by a recombinase excision event and the Gene Writer functions to insert the minicircle DNA by a recombinase integration event. In some embodiments, the excision event and integration event are catalyzed by the same enzyme, e.g., by the same serine recombinase. In some embodiments, the cassette for excision from a vector is flanked by attL and attR sites and the excision event results in the generation of an attB or attP site that is used for integration at a cognate genomic attP or attB site. In some embodiments, the excision event involving attL and attR sites is catalyzed by the addition of a recombination directionality factor (RDF) that enables the Gene Writer recombinase polypeptide to perform the excision. In some embodiments, the Gene Writer recombinase polypeptide functions to catalyze an integration event in the absence of an RDF.

### Long Terminal Repeats (LTRs)

In some embodiments, a template RNA described herein comprises an LTR, e.g., comprises two LTRs. The two LTRs may have identical sequences or may have sequence differences relative to one another. In some embodiments, the LTRs are lentiviral LTRs. In some embodiments, the LTRs are located at the two ends of the template RNA.

In some embodiments, the LTR comprises one or more of (e.g., all of) U3, R, and U5. In some embodiments, the LTR is a wild-type LTR. In other embodiments, the LTR comprises one or more sequence difference (e.g., deletion or substitution) compared to a corresponding wild-type LTR. In some embodiments, the LTR comprises reduced (e.g., abrogated) promoter and/or enhancer activity compared to a corresponding wild-type LTR. In some embodiments, the LTR comprises a deletion of U3, e.g., in the U3 of the 3′ LTR of the viral genome, which corresponds to the 5′ LTR after one round of reverse transcription. In some embodiments, the LTR is a self-inactivating LTR, e.g., as described in Cesana et al. “Uncovering and Dissecting the Genotoxicity of Self-inactivating Lentiviral Vectors In Vivo” doi:10.1038/mt.2014.3, which is herein incorporated by reference in its entirety.

### Linkers

In some embodiments, domains of the compositions and systems described herein (e.g., the recombinase domain and/or DNA recognition domains of a recombinase polypeptide, e.g., as described herein) may be joined by a linker. A composition described herein comprising a linker element has the general form S1-L-S2, wherein S1 and S2 may be the same or different and represent two domain moieties (e.g., each a polypeptide or nucleic acid domain) associated with one another by the linker. In some embodiments, a linker may connect two polypeptides. In some embodiments, a linker may connect two nucleic acid molecules. In some embodiments, a linker may connect a polypeptide and a nucleic acid molecule. A linker may be a chemical bond, e.g., one or more covalent bonds or non-covalent bonds. A linker may be flexible, rigid, and/or cleavable. In some embodiments, the linker is a peptide linker. Generally, a peptide linker is at least 2, 3, 4, 5, 6, 7, 8, 9, 10 or more amino acids in length, e.g., 2-50 amino acids in length, 2-30 amino acids in length.

The most commonly used flexible linkers have sequences consisting primarily of stretches of Gly and Ser residues (“GS” linker). Flexible linkers may be useful for joining domains that require a certain degree of movement or interaction and may include small, non-polar (e.g. Gly) or polar (e.g. Ser or Thr) amino acids. Incorporation of Ser or Thr can also maintain the stability of the linker in aqueous solutions by forming hydrogen bonds with the water molecules, and therefore reduce unfavorable interactions between the linker and the other moieties. Examples of such linkers include those having the structure [GGS]^{≥1} or [GGGS]^{≥1}. Rigid linkers are useful to keep a fixed distance between domains and to maintain their independent functions. Rigid linkers may also be useful when a spatial separation of the domains is critical to preserve the stability or bioactivity of one or more components in the agent. Rigid linkers may have an alpha helix-structure or Pro-rich sequence, (XP)n, with X designating any amino acid, preferably Ala, Lys, or Glu. Cleavable linkers may release free functional domains in vivo. In some embodiments, linkers may be cleaved under specific conditions, such as the presence of reducing reagents or proteases. In vivo cleavable linkers may utilize the reversible nature of a disulfide bond. One example includes a thrombin-sensitive sequence (e.g., PRS) between the two Cys residues. In vitro thrombin treatment of CPRSC results in the cleavage of the thrombin-sensitive sequence, while the reversible disulfide linkage remains intact. Such linkers are known and described, e.g., in Chen et al. 2013. Fusion Protein Linkers: Property, Design and Functionality. Adv Drug Deliv Rev. 65(10): 1357-1369. In vivo cleavage of linkers in compositions described herein may also be carried out by proteases that are expressed in vivo under pathological conditions (e.g. cancer or inflammation), in specific cells or tissues, or constrained within certain cellular compartments. The specificity of many proteases offers slower cleavage of the linker in constrained compartments.

In some embodiments the amino acid linkers are (or are homologous to) the endogenous amino acids that exist between such domains in a native polypeptide. In some embodiments the endogenous amino acids that exist between such domains are substituted but the length is unchanged from the natural length. In some embodiments, additional amino acid residues are added to the naturally existing amino acid residues between domains.

In some embodiments, the amino acid linkers are designed computationally or screened to maximize protein function (Anad et al., FEBS Letters, 587:19, 2013).

In some embodiments, a Gene Writer polypeptide may comprise a linker, e.g., a peptide linker, e.g., a linker as described in Table 7. In some embodiments, a Gene Writer polypeptide comprises a flexible linker.

TABLE 7
Exemplary linker sequences
Amino Acid Sequence

GGS
GGSGGS
GGSGGSGGS
GGSGGSGGSGGS
GGSGGSGGSGGSGGS
GGSGGSGGSGGSGGSGGS
GGGGS
GGGGSGGGGS
GGGGSGGGGSGGGGS
GGGGSGGGGSGGGGSGGGGS
GGGGSGGGGSGGGGSGGGGSGGGGS
GGGGSGGGGSGGGGSGGGGSGGGGSGGGGS
GGG
GGGG
GGGGG
GGGGGG
GGGGGGG
GGGGGGGG
GSS
GSSGSS
GSSGSSGSS
GSSGSSGSSGSS
GSSGSSGSSGSSGSS
GSSGSSGSSGSSGSSGSS
EAAAK
EAAAKEAAAK
EAAAKEAAAKEAAAK
EAAAKEAAAKEAAAKEAAAK
EAAAKEAAAKEAAAKEAAAKEAAAK
EAAAKEAAAKEAAAKEAAAKEAAAKEAAAK
PAP
PAPAP
PAPAPAP
PAPAPAPAP
PAPAPAPAPAP
PAPAPAPAPAPAP
GGSGGG
GGGGGS
GGSGSS
GSSGGS
GGSEAAAK
EAAAKGGS
GGSPAP
PAPGGS
GGGGSS
GSSGGG
GGGEAAAK
EAAAKGGG
GGGPAP
PAPGGG
GSSEAAAK
EAAAKGSS
GSSPAP
PAPGSS
EAAAKPAP
PAPEAAAK
GGSGGGGSS
GGSGSSGGG
GGGGGSGSS
GGGGSSGGS
GSSGGSGGG
GSSGGGGGS
GGSGGGEAAAK
GGSEAAAKGGG
GGGGGSEAAAK
GGGEAAAKGGS
EAAAKGGSGGG
EAAAKGGGGGS
GGSGGGPAP
GGSPAPGGG
GGGGGSPAP
GGGPAPGGS
PAPGGSGGG
PAPGGGGGS
GGSGSSEAAAK
GGSEAAAKGSS
GSSGGSEAAAK
GSSEAAAKGGS
EAAAKGGSGSS
EAAAKGSSGGS
GGSGSSPAP
GGSPAPGSS
GSSGGSPAP
GSSPAPGGS
PAPGGSGSS
PAPGSSGGS
GGSEAAAKPAP
GGSPAPEAAAK
EAAAKGGSPAP
EAAAKPAPGGS
PAPGGSEAAAK
PAPEAAAKGGS
GGGGSSEAAAK
GGGEAAAKGSS
GSSGGGEAAAK
GSSEAAAKGGG
EAAAKGGGGSS
EAAAKGSSGGG
GGGGSSPAP
GGGPAPGSS
GSSGGGPAP
GSSPAPGGG
PAPGGGGSS
PAPGSSGGG
GGGEAAAKPAP
GGGPAPEAAAK
EAAAKGGGPAP
EAAAKPAPGGG
PAPGGGEAAAK
PAPEAAAKGGG
GSSEAAAKPAP
GSSPAPEAAAK
EAAAKGSSPAP
EAAAKPAPGSS
PAPGSSEAAAK
PAPEAAAKGSS
AEAAAKEAAAKEAAAKEAAAKALEAEAAAKEAAAKEAAAKEAAAKA
GGGGSEAAAKGGGGS
EAAAKGGGGSEAAAK
SGSETPGTSESATPES
GSAGSAAGSGEF
SGGSSGGSSGSETPGTSESATPESSGGSSGGSS

Additional Gene Writer characteristics

In some embodiments, the Gene Writer system may result in complete writing without requiring endogenous host factors. In some embodiments, the system may result in complete writing without the need for DNA repair. In some embodiments, the system may result in complete writing without eliciting a DNA damage response.

In some embodiments, the system does not require DNA repair by the NHEJ pathway, homologous recombination repair pathway, base excision repair pathway, or any combination thereof. Participation by a DNA repair pathway can be assayed, for example, via the application of DNA repair pathway inhibitors or DNA repair pathway deficient cell lines. For example, when applying DNA repair pathway inhibitors, PrestoBlue cell viability assay can be performed first to determine the toxicity of the inhibitors and whether any normalization should be applied. SCR7 is an inhibitor for NHEJ, which can be applied at a series of dilutions during Gene Writer™ delivery. PARP protein is a nuclear enzyme that binds as homodimers to both single- and double-strand breaks. Thus, its inhibitors can be used in the test of relevant DNA repair pathways, including homologous recombination repair pathway and base excision repair pathway. The experiment procedure is the same with that of SCR7. Cell lines with deficient core proteins of nucleotide excision repair (NER) pathway can be used to test the effect of NER on Gene Writing™. After the delivery of the Gene Writer™ system into the cell, ddPCR can used to evaluate the insertion of a heterologous object sequence in the context of inhibition of DNA repair pathways. Sequencing analysis can also be performed to evaluate whether certain DNA repair pathways play a role. In some embodiments, Gene Writing™ into the genome is not decreased by the knockdown of a DNA repair pathway described herein. In some embodiments, Gene Writing™ into the genome is not decreased by more than 50% by the knockdown of the DNA repair pathway.

### Circular RNAs in Gene Writing Systems

It is contemplated that it may be useful to employ circular and/or linear RNA states during the formulation, delivery, or Gene Writing reaction within the target cell. Thus, in some embodiments of any of the aspects described herein, a Gene Writing system comprises one or more circular RNAs (circRNAs). In some embodiments of any of the aspects described herein, a Gene Writing system comprises one or more linear RNAs. In some embodiments, a nucleic acid as described herein (e.g., a nucleic acid molecule encoding a Gene Writer polypeptide, or both) is a circRNA. In some embodiments, a circular RNA molecule encodes the Gene Writer polypeptide. In some embodiments, the circRNA molecule encoding the Gene Writer polypeptide is delivered to a host cell. In some embodiments, a circular RNA molecule encodes a recombinase, e.g., as described herein. In some embodiments, the circRNA molecule encoding the recombinase is delivered to a host cell. In some embodiments, the circRNA molecule encoding the Gene Writer polypeptide is linearized (e.g., in the host cell) prior to translation.

Circular RNAs (circRNAs) have been found to occur naturally in cells and have been found to have diverse functions, including both non-coding and protein coding roles in human cells. It has been shown that a circRNA can be engineered by incorporating a self-splicing intron into an RNA molecule (or DNA encoding the RNA molecule) that results in circularization of the RNA, and that an engineered circRNA can have enhanced protein production and stability (Wesselhoeft et al. Nature Communications 2018). In some embodiments, the Gene Writer™ polypeptide is encoded as circRNA. In certain embodiments, the template nucleic acid is a DNA, such as a dsDNA or ssDNA.

In some embodiments, the circRNA comprises one or more ribozyme sequence. In some embodiments, the ribozyme sequence is activated for autocleavage, e.g., in a host cell, e.g., thereby resulting in linearization of the circRNA. In some embodiments, the ribozyme is activated when the concentration of magnesium reaches a sufficient level for cleavage, e.g., in a host cell. In some embodiments the circRNA is maintained in a low magnesium environment prior to delivery to the host cell. In some embodiments, the ribozyme is a protein-responsive ribozyme. In some embodiments, the ribozyme is a nucleic acid-responsive ribozyme.

In some embodiments, the circRNA is linearized in the nucleus of a target cell. In some embodiments, linearization of a circRNA in the nucleus of a cell involves components present in the nucleus of the cell, e.g., to activate a cleavage event. For example, the B2 and ALU retrotransposons contain self-cleaving ribozymes whose activity is enhanced by interaction with the Polycomb protein, EZH2 (Hernandez et al. PNAS 117(1):415-425 (2020)). Thus, in some embodiments, a ribozyme, e.g., a ribozyme from a B2 or ALU element, that is responsive to a nuclear element, e.g., a nuclear protein, e.g., a genome-interacting protein, e.g., an epigenetic modifier, e.g., EZH2, is incorporated into a circRNA, e.g., of a Gene Writing system. In some embodiments, nuclear localization of the circRNA results in an increase in autocatalytic activity of the ribozyme and linearization of the circRNA.

In some embodiments, an inducible ribozyme (e.g., in a circRNA as described herein) is created synthetically, for example, by utilizing a protein ligand-responsive aptamer design. A system for utilizing the satellite RNA of tobacco ringspot virus hammerhead ribozyme with an MS2 coat protein aptamer has been described (Kennedy et al. Nucleic Acids Res 42(19):12306-12321 (2014), incorporated herein by reference in its entirety) that results in activation of the ribozyme activity in the presence of the MS2 coat protein. In embodiments, such a system responds to protein ligand localized to the cytoplasm or the nucleus. In some embodiments the protein ligand is not MS2. Methods for generating RNA aptamers to target ligands have been described, for example, based on the systematic evolution of ligands by exponential enrichment (SELEX) (Tuerk and Gold, Science 249(4968):505-510 (1990); Ellington and Szostak, Nature 346(6287):818-822 (1990); the methods of each of which are incorporated herein by reference) and have, in some instances, been aided by in silico design (Bell et al. PNAS 117(15):8486-8493, the methods of which are incorporated herein by reference). Thus, in some embodiments, an aptamer for a target ligand is generated and incorporated into a synthetic ribozyme system, e.g., to trigger ribozyme-mediated cleavage and circRNA linearization, e.g., in the presence of the protein ligand. In some embodiments, circRNA linearization is triggered in the cytoplasm, e.g., using an aptamer that associates with a ligand in the cytoplasm. In some embodiments, circRNA linearization is triggered in the nucleus, e.g., using an aptamer that associates with a ligand in the nucleus. In embodiments, the ligand comprises an epigenetic modifier or a transcription factor. In some embodiments the ligand that triggers linearization is present at higher levels in on-target cells than off-target cells.

It is further contemplated that a nucleic acid-responsive ribozyme system can be employed for circRNA linearization. For example, biosensors that sense defined target nucleic acid molecules to trigger ribozyme activation are described, e.g., in Penchovsky (Biotechnology Advances 32(5):1015-1027 (2014), incorporated herein by reference). By these methods, a ribozyme naturally folds into an inactive state and is only activated in the presence of a defined target nucleic acid molecule (e.g., an RNA molecule). In some embodiments, a circRNA of a Gene Writing system comprises a nucleic acid-responsive ribozyme that is activated in the presence of a defined target nucleic acid, e.g., an RNA, e.g., an mRNA, miRNA, guide RNA, gRNA, sgRNA, ncRNA, lncRNA, tRNA, snRNA, or mtRNA. In some embodiments the nucleic acid that triggers linearization is present at higher levels in on-target cells than off-target cells.

In some embodiments of any of the aspects herein, a Gene Writing system incorporates one or more ribozymes with inducible specificity to a target tissue or target cell of interest, e.g., a ribozyme that is activated by a ligand or nucleic acid present at higher levels in a target tissue or target cell of interest. In some embodiments, the Gene Writing system incorporates a ribozyme with inducible specificity to a subcellular compartment, e.g., the nucleus, nucleolus, cytoplasm, or mitochondria. In some embodiments, the ribozyme that is activated by a ligand or nucleic acid present at higher levels in the target subcellular compartment. In some embodiments, an RNA component of a Gene Writing system is provided as circRNA, e.g., that is activated by linearization. In some embodiments, linearization of a circRNA encoding a Gene Writing polypeptide activates the molecule for translation. In some embodiments, a signal that activates a circRNA component of a Gene Writing system is present at higher levels in on-target cells or tissues, e.g., such that the system is specifically activated in these cells.

In some embodiments, an RNA component of a Gene Writing system is provided as a circRNA that is inactivated by linearization. In some embodiments, a circRNA encoding the Gene Writer polypeptide is inactivated by cleavage and degradation. In some embodiments, a circRNA encoding the Gene Writing polypeptide is inactivated by cleavage that separates a translation signal from the coding sequence of the polypeptide. In some embodiments, a signal that inactivates a circRNA component of a Gene Writing system is present at higher levels in off-target cells or tissues, such that the system is specifically inactivated in these cells.

### Evolved Variants of Gene Writers

In some embodiments, the invention provides evolved variants of Gene Writers. Evolved variants can, in some embodiments, be produced by mutagenizing a reference Gene Writer, or one of the fragments or domains comprised therein. In some embodiments, one or more of the domains (e.g., the catalytic domain or DNA binding domain (e.g., target binding domain or template binding domain), including, for example, sequence-guided DNA binding elements) is evolved. One or more of such evolved variant domains can, in some embodiments, be evolved alone or together with other domains. An evolved variant domain or domains may, in some embodiments, be combined with unevolved cognate component(s) or evolved variants of the cognate component(s), e.g., which may have been evolved in either a parallel or serial manner.

In some embodiments, the process of mutagenizing a reference Gene Writer, or fragment or domain thereof, comprises mutagenizing the reference Gene Writer or fragment or domain thereof. In embodiments, the mutagenesis comprises a continuous evolution method (e.g., PACE) or non-continuous evolution method (e.g., PANCE), e.g., as described herein. In some embodiments, the evolved Gene Writer, or a fragment or domain thereof (e.g., a DNA binding domain, e.g., a target binding domain or a template binding domain), comprises one or more amino acid variations introduced into its amino acid sequence relative to the amino acid sequence of the reference Gene Writer, or fragment or domain thereof. In embodiments, amino acid sequence variations may include one or more mutated residues (e.g., conservative substitutions, non-conservative substitutions, or a combination thereof) within the amino acid sequence of a reference Gene Writer, e.g., as a result of a change in the nucleotide sequence encoding the gene writer that results in, e.g., a change in the codon at any particular position in the coding sequence, the deletion of one or more amino acids (e.g., a truncated protein), the insertion of one or more amino acids, or any combination of the foregoing. The evolved variant Gene Writer may include variants in one or more components or domains of the Gene Writer (e.g., variants introduced into a catalytic domain, DNA binding domain, or combinations thereof).

In some aspects, the invention provides Gene Writers, systems, kits, and methods using or comprising an evolved variant of a Gene Writer, e.g., employs an evolved variant of a Gene Writer or a Gene Writer produced or produceable by PACE or PANCE. In embodiments, the unevolved reference Gene Writer is a Gene Writer as disclosed herein.

The term “phage-assisted continuous evolution (PACE),” as used herein, generally refers to continuous evolution that employs phage as viral vectors. Examples of PACE technology have been described, for example, in International PCT Application No. PCT/US 2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; and International PCT Application, PCT/US2016/027795, filed Apr. 15, 2016, published as WO 2016/168631 on Oct. 20, 2016, the entire contents of each of which are incorporated herein by reference.

The term “phage-assisted non-continuous evolution (PANCE),” as used herein, generally refers to non-continuous evolution that employs phage as viral vectors. Examples of PANCE technology have been described, for example, in Suzuki T. et al, Crystal structures reveal an elusive functional domain of pyrrolysyl-tRNA synthetase, Nat Chem Biol. 13(12): 1261-1266 (2017), incorporated herein by reference in its entirety. Briefly, PANCE is a technique for rapid in vivo directed evolution using serial flask transfers of evolving selection phage (SP), which contain a gene of interest to be evolved, across fresh host cells (e.g., E. coli cells). Genes inside the host cell may be held constant while genes contained in the SP continuously evolve. Following phage growth, an aliquot of infected cells may be used to transfect a subsequent flask containing host E. coli. This process can be repeated and/or continued until the desired phenotype is evolved, e.g., for as many transfers as desired.

Methods of applying PACE and PANCE to Gene Writers may be readily appreciated by the skilled artisan by reference to, inter alia, the foregoing references. Additional exemplary methods for directing continuous evolution of genome-modifying proteins or systems, e.g., in a population of host cells, e.g., using phage particles, can be applied to generate evolved variants of Gene Writers, or fragments or subdomains thereof. Non-limiting examples of such methods are described in International PCT Application, PCT/US2009/056194, filed Sep. 8, 2009, published as WO 2010/028347 on Mar. 11, 2010; International PCT Application, PCT/US2011/066747, filed Dec. 22, 2011, published as WO 2012/088381 on Jun. 28, 2012; U.S. Pat. No. 9,023,594, issued May 5, 2015; U.S. Pat. No. 9,771,574, issued Sep. 26, 2017; U.S. Pat. No. 9,394,537, issued Jul. 19, 2016; International PCT Application, PCT/US2015/012022, filed Jan. 20, 2015, published as WO 2015/134121 on Sep. 11, 2015; U.S. Pat. No. 10,179,911, issued Jan. 15, 2019; International Application No. PCT/US2019/37216, filed Jun. 14, 2019, International Patent Publication WO 2019/023680, published Jan. 31, 2019, International PCT Application, PCT/US2016/027795, filed Apr. 15, 2016, published as WO 2016/168631 on Oct. 20, 2016, and International Patent Publication No. PCT/US2019/47996, filed Aug. 23, 2019, each of which is incorporated herein by reference in its entirety.

In some non-limiting illustrative embodiments, a method of evolution of a evolved variant Gene Writer, of a fragment or domain thereof, comprises: (a) contacting a population of host cells with a population of viral vectors comprising the gene of interest (the starting Gene Writer or fragment or domain thereof), wherein: (1) the host cell is amenable to infection by the viral vector; (2) the host cell expresses viral genes required for the generation of viral particles; (3) the expression of at least one viral gene required for the production of an infectious viral particle is dependent on a function of the gene of interest; and/or (4) the viral vector allows for expression of the protein in the host cell, and can be replicated and packaged into a viral particle by the host cell. In some embodiments, the method comprises (b) contacting the host cells with a mutagen, using host cells with mutations that elevate mutation rate (e.g., either by carrying a mutation plasmid or some genome modification—e.g., proofing-impaired DNA polymerase, SOS genes, such as UmuC, UmuD′, and/or RecA, which mutations, if plasmid-bound, may be under control of an inducible promoter), or a combination thereof. In some embodiments, the method comprises (c) incubating the population of host cells under conditions allowing for viral replication and the production of viral particles, wherein host cells are removed from the host cell population, and fresh, uninfected host cells are introduced into the population of host cells, thus replenishing the population of host cells and creating a flow of host cells. In some embodiments, the cells are incubated under conditions allowing for the gene of interest to acquire a mutation. In some embodiments, the method further comprises (d) isolating a mutated version of the viral vector, encoding an evolved gene product (e.g., an evolved variant Gene Writer, or fragment or domain thereof), from the population of host cells.

The skilled artisan will appreciate a variety of features employable within the above-described framework. For example, in some embodiments, the viral vector or the phage is a filamentous phage, for example, an M13 phage, e.g., an M13 selection phage. In certain embodiments, the gene required for the production of infectious viral particles is the M13 gene III (gIII). In embodiments, the phage may lack a functional gIII, but otherwise comprise gI, gII, gIV, gV, gVI, gVII, gVIII, gIX, and a gX. In some embodiments, the generation of infectious VSV particles involves the envelope protein VSV-G. Various embodiments can use different retroviral vectors, for example, Murine Leukemia Virus vectors, or Lentiviral vectors, e.g., packaged with VSV-G envelope protein, e.g., as a substitute for the native envelope protein of the virus.

In some embodiments, host cells are incubated according to a suitable number of viral life cycles, e.g., at least 10, at least 20, at least 30, at least 40, at least 50, at least 100, at least 200, at least 300, at least 400, at least, 500, at least 600, at least 700, at least 800, at least 900, at least 1000, at least 1250, at least 1500, at least 1750, at least 2000, at least 2500, at least 3000, at least 4000, at least 5000, at least 7500, at least 10000, or more consecutive viral life cycles, which in on illustrative and non-limiting examples of M13 phage is 10-20 minutes per virus life cycle. Similarly, conditions can be modulated to adjust the time a host cell remains in a population of host cells, e.g., about 10, about 11, about 12, about 13, about 14, about 15, about 16, about 17, about 18, about 19, about 20, about 21, about 22, about 23, about 24, about 25, about 30, about 35, about 40, about 45, about 50, about 55, about 60, about 70, about 80, about 90, about 100, about 120, about 150, or about 180 minutes. Host cell populations can be controlled in part by density of the host cells, or, in some embodiments, the host cell density in an inflow, e.g., 10³ cells/ml, about 10⁴ cells/ml, about 10⁵ cells/ml, about 5-10⁵ cells/ml, about 10⁶ cells/ml, about 5-10⁶ cells/ml, about 10⁷ cells/ml, about 5-10⁷ cells/ml, about 10⁸ cells/ml, about 5-10⁸ cells/ml, about 10⁹ cells/ml, about 5·10⁹ cells/ml, about 10¹⁰ cells/ml, or about 5·10¹⁰ cells/ml.

### Nucleic Acids

### Promoters

In some embodiments, one or more promoter or enhancer elements are operably linked to a nucleic acid encoding a Gene Writer polypeptide or a template nucleic acid, e.g., that controls expression of the heterologous object sequence. In certain embodiments, the one or more promoter or enhancer elements comprise cell-type or tissue specific elements. In some embodiments, the promoter or enhancer is the same or derived from the promoter or enhancer that naturally controls expression of the heterologous object sequence. For example, the ornithine transcarbomylase promoter and enhancer may be used to control expression of the ornithine transcarbomylase gene in a system or method provided by the invention for correcting ornithine transcarbomylase deficiencies. In some embodiments, the promoter is a promoter of Table 8 or a functional fragment or variant thereof.

Exemplary tissue specific promoters that are commercially available can be found, for example, at a uniform resource locator (e.g., https://www.invivogen.com/tissue-specific-promoters). In some embodiments, a promoter is a native promoter or a minimal promoter, e.g., which consists of a single fragment from the 5′ region of a given gene. In some embodiments, a native promoter comprises a core promoter and its natural 5′ UTR. In some embodiments, the 5′ UTR comprises an intron. In other embodiments, these include composite promoters, which combine promoter elements of different origins or were generated by assembling a distal enhancer with a minimal promoter of the same origin. In some embodiments, a tissue-specific expression-control sequence(s) comprises one or more of the sequences in Table 2 or Table 3 of PCT Publication No. WO2020014209 (incorporated herein by reference in its entirety).

Exemplary cell or tissue specific promoters are provided in the tables, below, and exemplary nucleic acid sequences encoding them are known in the art and can be readily accessed using a variety of resources, such as the NCBI database, including RefSeq, as well as the Eukaryotic Promoter Database (http://epd.epfl.ch//index.php).

TABLE 8
Exemplary cell or tissue-specific promoters

| Promoter | Target cells |
|---|---|
| B29 Promoter | B cells |
| CD14 Promoter | Monocytic Cells |
| CD43 Promoter | Leukocytes and platelets |
| CD45 Promoter | Hematopoeitic cells |
| CD68 promoter | macrophages |
| Desmin promoter | muscle cells |
| Elastase-1 | pancreatic acinar cells |
| promoter | |
| Endoglin promoter | endothelial cells |
| fibronectin | differentiating cells, healing |
| promoter | tissue |
| Flt-1 promoter | endothelial cells |
| GFAP promoter | Astrocytes |
| GPIIB promoter | megakaryocytes |
| ICAM-2 Promoter | Endothelial cells |
| INF-Beta promoter | Hematopoeitic cells |
| Mb promoter | muscle cells |
| Nphs1 promoter | podocytes |
| OG-2 promoter | Osteoblasts, Odonblasts |
| SP-B promoter | Lung |
| Syn1 promoter | Neurons |
| WASP promoter | Hematopoeitic cells |
| SV40/bAlb | Liver |
| promoter | |
| SV40/bAlb | Liver |
| promoter | |
| SV40/Cd3 | Leukocytes and platelets |
| promoter | |
| SV40/CD45 | hematopoeitic cells |
| promoter | |
| NSE/RU5′ | Mature Neurons |
| promoter | |

TABLE 9
Additional exemplary cell or tissue-specific promoters

| Promoter | Gene Description | Gene Specificity |
|---|---|---|
| APOA2 | Apolipoprotein A-II | Hepatocytes (from hepatocyte |
| | | progenitors) |
| SERPINA | Serpin peptidase inhibitor, clade A | Hepatocytes (from |
| 1 (hAAT) | (alpha-1 antiproteinase, antitrypsin), | definitive endoderm stage) |
| | member 1 (also named alpha 1 | |
| | anti-tryps in) | |
| CYP3A | Cytochrome P450, family 3, | Mature Hepatocytes |
| | subfamily A, polypeptide | |
| MIR122 | MicroRNA 122 | Hepatocytes |
| | | (from early stage embryonic |
| | | liver cells) |
| | | and endoderm |

Pancreatic specific promoters

| | | |
|---|---|---|
| INS | Insulin | Pancreatic beta cells |
| | | (from definitive endoderm stage) |
| IRS2 | Insulin receptor substrate 2 | Pancreatic beta cells |
| Pdx1 | Pancreatic and duodenal | Pancreas |
| | homeobox 1 | (from definitive endoderm stage) |
| Alx3 | Aristaless-like homeobox 3 | Pancreatic beta cells |
| | | (from definitive endoderm stage) |
| Ppy | Pancreatic polypeptide | PP pancreatic cells |
| | | (gamma cells) |

Cardiac specific promoters

| | | |
|---|---|---|
| Myh6 | Myosin, heavy chain 6, cardiac | Late differentiation marker of cardiac |
| (aMHC) | muscle, alpha | muscle cells (atrial specificity) |
| MYL2 | Myosin, light chain 2, regulatory, | Late differentiation marker of cardiac |
| (MLC-2v) | cardiac, slow | muscle cells (ventricular specificity) |
| ITNNl3 | Troponin I type 3 (cardiac) | Cardiomyocytes |
| (cTnl) | | (from immature state) |
| ITNNl3 | Troponin I type 3 (cardiac) | Cardiomyocytes |
| (cTnl) | | (from immature state) |
| NPPA | Natriuretic peptide precursor A (also | Atrial specificity in adult cells |
| (ANF) | named Atrial Natriuretic Factor) | |
| Slc8a1 | Solute carrier family 8 | Cardiomyocytes from early |
| (Ncx1) | (sodium/calcium exchanger), member | developmental stages |
| | 1 | |

CNS specific promoters

| | | |
|---|---|---|
| SYN1 | Synapsin I | Neurons |
| (hSyn) | | |
| GFAP | Glial fibrillary acidic protein | Astrocytes |
| INA | Internexin neuronal intermediate | Neuroprogenitors |
| | filament protein, alpha (a-internexin) | |
| NES | Nestin | Neuroprogenitors and ectoderm |
| MOBP | Myelin-associated oligodendrocyte | Oligodendrocytes |
| | basic protein | |
| MBP | Myelin basic protein | Oligodendrocytes |
| TH | Tyrosine hydroxylase | Dopaminergic neurons |
| FOXA2 | Forkhead box A2 | Dopaminergic neurons (also used as a |
| (HNF3 | | marker of endoderm) |
| beta) | | |

Skin specific promoters

| | | |
|---|---|---|
| FLG | Filaggrin | Keratinocytes from granular layer |
| K14 | Keratin 14 | Keratinocytes from granular |
| | | and basal layers |
| TGM3 | Transglutaminase 3 | Keratinocytes from granular layer |

Immune cell specific promoters

| | | |
|---|---|---|
| ITGAM | Integrin, alpha M (complement | Monocytes, macrophages, granulocytes, |
| (CD11B) | component 3 receptor 3 subunit) | natural killer cells |

Urogential cell specific promoters

| | | |
|---|---|---|
| Pbsn | Probasin | Prostatic epithelium |
| Upk2 | Uroplakin 2 | Bladder |
| Sbp | Spermine binding protein | Prostate |
| Fer1l4 | Fer-1-like 4 | Bladder |

Endothelial cell specific promoters

| | | |
|---|---|---|
| ENG | Endoglin | Endothelial cells |

Pluripotent and embryonic cell specific promoters

| | | |
|---|---|---|
| Oct4 | POU class 5 homeobox 1 | Pluripotent cells |
| (POU5F1) | | (germ cells, ES cells, iPS cells) |
| NANOG | Nanog homeobox | Pluripotent cells |
| | | (ES cells, iPS cells) |
| Synthetic | Synthetic promoter based on a Oct-4 | Pluripotent cells (ES cells, iPS cells) |
| Oct4 | core enhancer element | |
| T | Brachyury | Mesoderm |
| brachyury | | |
| NES | Nestin | Neuroprogenitors and Ectoderm |
| SOX17 | SRY (sex determining region Y)-box | Endoderm |
| | 17 | |
| FOXA2 | Forkhead box A2 | Endoderm (also used as a marker of |
| (HNFJ | | dopaminergic neurons) |
| beta) | | |
| MIR122 | MicroRNA 122 | Endoderm and hepatocytes |
| | | (from early stage embryonic liver cells~ |

Depending on the host/vector system utilized, any of a number of suitable transcription and translation control elements, including constitutive and inducible promoters, transcription enhancer elements, transcription terminators, etc. may be used in the expression vector (see e.g., Bitter et al. (1987) Methods in Enzymology, 153:516-544; incorporated herein by reference in its entirety).

In some embodiments, a nucleic acid encoding a Gene Writer or template nucleic acid is operably linked to a control element, e.g., a transcriptional control element, such as a promoter. The transcriptional control element may, in some embodiment, be functional in either a eukaryotic cell, e.g., a mammalian cell; or a prokaryotic cell (e.g., bacterial or archaeal cell). In some embodiments, a nucleotide sequence encoding a polypeptide is operably linked to multiple control elements, e.g., that allow expression of the nucleotide sequence encoding the polypeptide in both prokaryotic and eukaryotic cells.

For illustration purposes, examples of spatially restricted promoters include, but are not limited to, neuron-specific promoters, adipocyte-specific promoters, cardiomyocyte-specific promoters, smooth muscle-specific promoters, photoreceptor-specific promoters, etc. Neuron-specific spatially restricted promoters include, but are not limited to, a neuron-specific enolase (NSE) promoter (see, e.g., EMBL HSENO2, X51956); an aromatic amino acid decarboxylase (AADC) promoter, a neurofilament promoter (see, e.g., GenBank HUMNFL, L04147); a synapsin promoter (see, e.g., GenBank HUMSYNIB, M55301); a thy-1 promoter (see, e.g., Chen et al. (1987) Cell 51:7-19; and Llewellyn, et al. (2010) Nat. Med. 16(10):1161-1166); a serotonin receptor promoter (see, e.g., GenBank 562283); a tyrosine hydroxylase promoter (TH) (see, e.g., Oh et al. (2009) Gene Ther 16:437; Sasaoka et al. (1992) Mol. Brain Res. 16:274; Boundy et al. (1998) J. Neurosci. 18:9989; and Kaneda et al. (1991) Neuron 6:583-594); a GnRH promoter (see, e.g., Radovick et al. (1991) Proc. Natl. Acad. Sci. USA 88:3402-3406); an L7 promoter (see, e.g., Oberdick et al. (1990) Science 248:223-226); a DNMT promoter (see, e.g., Bartge et al. (1988) Proc. Natl. Acad. Sci. USA 85:3648-3652); an enkephalin promoter (see, e.g., Comb et al. (1988) EMBO J. 17:3793-3805); a myelin basic protein (MBP) promoter; a Ca2+-calmodulin-dependent protein kinase II-alpha (CamKIIa) promoter (see, e.g., Mayford et al. (1996) Proc. Natl. Acad. Sci. USA 93:13250; and Casanova et al. (2001) Genesis 31:37); a CMV enhancer/platelet-derived growth factor-O promoter (see, e.g., Liu et al. (2004) Gene Therapy 11:52-60); and the like.

Adipocyte-specific spatially restricted promoters include, but are not limited to, the aP2 gene promoter/enhancer, e.g., a region from −5.4 kb to +21 bp of a human aP2 gene (see, e.g., Tozzo et al. (1997) Endocrinol. 138:1604; Ross et al. (1990) Proc. Natl. Acad. Sci. USA 87:9590; and Pavjani et al. (2005) Nat. Med. 11:797); a glucose transporter-4 (GLUT4) promoter (see, e.g., Knight et al. (2003) Proc. Natl. Acad. Sci. USA 100:14725); a fatty acid translocase (FAT/CD36) promoter (see, e.g., Kuriki et al. (2002) Biol. Pharm. Bull. 25:1476; and Sato et al. (2002) J. Biol. Chem. 277:15703); a stearoyl-CoA desaturase-1 (SCD1) promoter (Tabor et al. (1999) J. Biol. Chem. 274:20603); a leptin promoter (see, e.g., Mason et al. (1998) Endocrinol. 139:1013; and Chen et al. (1999) Biochem. Biophys. Res. Comm. 262:187); an adiponectin promoter (see, e.g., Kita et al. (2005) Biochem. Biophys. Res. Comm. 331:484; and Chakrabarti (2010) Endocrinol. 151:2408); an adipsin promoter (see, e.g., Platt et al. (1989) Proc. Natl. Acad. Sci. USA 86:7490); a resistin promoter (see, e.g., Seo et al. (2003) Molec. Endocrinol. 17:1522); and the like.

Cardiomyocyte-specific spatially restricted promoters include, but are not limited to, control sequences derived from the following genes: myosin light chain-2, a-myosin heavy chain, AE3, cardiac troponin C, cardiac actin, and the like. Franz et al. (1997) Cardiovasc. Res. 35:560-566; Robbins et al. (1995) Ann. N.Y. Acad. Sci. 752:492-505; Linn et al. (1995) Circ. Res. 76:584-591; Parmacek et al. (1994) Mol. Cell. Biol. 14:1870-1885; Hunter et al. (1993) Hypertension 22:608-617; and Sartorelli et al. (1992) Proc. Natl. Acad. Sci. USA 89:4047-4051.

Smooth muscle-specific spatially restricted promoters include, but are not limited to, an SM22a promoter (see, e.g., Akyurek et al. (2000) Mol. Med. 6:983; and U.S. Pat. No. 7,169,874); a smoothelin promoter (see, e.g., WO 2001/018048); an a-smooth muscle actin promoter; and the like. For example, a 0.4 kb region of the SM22a promoter, within which lie two CArG elements, has been shown to mediate vascular smooth muscle cell-specific expression (see, e.g., Kim, et al. (1997) Mol. Cell. Biol. 17, 2266-2278; Li, et al., (1996) J. Cell Biol. 132, 849-859; and Moessler, et al. (1996) Development 122, 2415-2425).

Photoreceptor-specific spatially restricted promoters include, but are not limited to, a rhodopsin promoter; a rhodopsin kinase promoter (Young et al. (2003) Ophthalmol. Vis. Sci. 44:4076); a beta phosphodiesterase gene promoter (Nicoud et al. (2007) J. Gene Med. 9:1015); a retinitis pigmentosa gene promoter (Nicoud et al. (2007) supra); an interphotoreceptor retinoid-binding protein (IRBP) gene enhancer (Nicoud et al. (2007) supra); an IRBP gene promoter (Yokoyama et al. (1992) Exp Eye Res. 55:225); and the like.

### Nonlimiting Exemplary Cell-Specific Promoters

Cell-specific promoters known in the art may be used to direct expression of a Gene Writer protein, e.g., as described herein. Nonlimiting exemplary mammalian cell-specific promoters have been characterized and used in mice expressing Cre recombinase in a cell-specific manner. Certain nonlimiting exemplary mammalian cell-specific promoters are listed in Table 1 of U.S. Pat. No. 9,845,481, incorporated herein by reference.

In some embodiments, the cell-specific promoter is a promoter that is active in plants. Many exemplary cell-specific plant promoters are known in the art. See, e.g., U.S. Pat. Nos. 5,097,025; 5,783,393; 5,880,330; 5,981,727; 7,557,264; 6,291,666; 7,132,526; and 7,323,622; and U.S. Publication Nos. 2010/0269226; 2007/0180580; 2005/0034192; and 2005/0086712, which are incorporated by reference herein in their entireties for any purpose.

In some embodiments, a vector as described herein comprises an expression cassette. The term “expression cassette”, as used herein, refers to a nucleic acid construct comprising nucleic acid elements sufficient for the expression of the nucleic acid molecule of the instant invention. Typically, an expression cassette comprises the nucleic acid molecule of the instant invention operatively linked to a promoter sequence. The term “operatively linked” refers to the association of two or more nucleic acid fragments on a single nucleic acid fragment so that the function of one is affected by the other. For example, a promoter is operatively linked with a coding sequence when it is capable of affecting the expression of that coding sequence (e.g., the coding sequence is under the transcriptional control of the promoter). Encoding sequences can be operatively linked to regulatory sequences in sense or antisense orientation. In certain embodiments, the promoter is a heterologous promoter. The term “heterologous promoter”, as used herein, refers to a promoter that is not found to be operatively linked to a given encoding sequence in nature. In certain embodiments, an expression cassette may comprise additional elements, for example, an intron, an enhancer, a polyadenylation site, a woodchuck response element (WRE), and/or other elements known to affect expression levels of the encoding sequence. A “promoter” typically controls the expression of a coding sequence or functional RNA. In certain embodiments, a promoter sequence comprises proximal and more distal upstream elements and can further comprise an enhancer element. An “enhancer” can typically stimulate promoter activity and may be an innate element of the promoter or a heterologous element inserted to enhance the level or tissue-specificity of a promoter. In certain embodiments, the promoter is derived in its entirety from a native gene. In certain embodiments, the promoter is composed of different elements derived from different naturally occurring promoters. In certain embodiments, the promoter comprises a synthetic nucleotide sequence. It will be understood by those skilled in the art that different promoters will direct the expression of a gene in different tissues or cell types, or at different stages of development, or in response to different environmental conditions or to the presence or the absence of a drug or transcriptional co-factor. Ubiquitous, cell-type-specific, tissue-specific, developmental stage-specific, and conditional promoters, for example, drug-responsive promoters (e.g., tetracycline-responsive promoters) are well known to those of skill in the art. Examples of promoter include, but are not limited to, the phosphoglycerate kinase (PKG) promoter, CAG (composite of the CMV enhancer the chicken beta actin promoter (CBA) and the rabbit beta globin intron.), NSE (neuronal specific enolase), synapsin or NeuN promoters, the SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), SFFV promoter, rous sarcoma virus (RSV) promoter, synthetic promoters, hybrid promoters, and the like. Other promoters can be of human origin or from other species, including from mice. Common promoters include, e.g., the human cytomegalovirus (CMV) immediate early gene promoter, the SV40 early promoter, the Rous sarcoma virus long terminal repeat, [beta]-actin, rat insulin promoter, the phosphoglycerate kinase promoter, the human alpha-1 antitrypsin (hAAT) promoter, the transthyretin promoter, the TBG promoter and other liver-specific promoters, the desmin promoter and similar muscle-specific promoters, the EF1-alpha promoter, the CAG promoter and other constitutive promoters, hybrid promoters with multi-tissue specificity, promoters specific for neurons like synapsin and glyceraldehyde-3-phosphate dehydrogenase promoter, all of which are promoters well known and readily available to those of skill in the art, can be used to obtain high-level expression of the coding sequence of interest. In addition, sequences derived from non-viral genes, such as the murine metallothionein gene, will also find use herein. Such promoter sequences are commercially available from, e.g., Stratagene (San Diego, CA). Additional exemplary promoter sequences are described, for example, in WO2018213786A1 (incorporated by reference herein in its entirety).

In some embodiments, the apolipoprotein E enhancer (ApoE) or a functional fragment thereof is used, e.g., to drive expression in the liver. In some embodiments, two copies of the ApoE enhancer or a functional fragment thereof is used. In some embodiments, the ApoE enhancer or functional fragment thereof is used in combination with a promoter, e.g., the human alpha-1 antitrypsin (hAAT) promoter.

In some embodiments, the regulatory sequences impart tissue-specific gene expression capabilities. In some cases, the tissue-specific regulatory sequences bind tissue-specific transcription factors that induce transcription in a tissue specific manner. Various tissue-specific regulatory sequences (e.g., promoters, enhancers, etc.) are known in the art. Exemplary tissue-specific regulatory sequences include, but are not limited to, the following tissue-specific promoters: a liver-specific thyroxin binding globulin (TBG) promoter, a insulin promoter, a glucagon promoter, a somatostatin promoter, a pancreatic polypeptide (PPY) promoter, a synapsin-1 (Syn) promoter, a creatine kinase (MCK) promoter, a mammalian desmin (DES) promoter, a α-myosin heavy chain (a-MHC) promoter, or a cardiac Troponin T (cTnT) promoter. Other exemplary promoters include Beta-actin promoter, hepatitis B virus core promoter, Sandig et al., Gene Ther., 3:1002-9 (1996); alpha-fetoprotein (AFP) promoter, Arbuthnot et al., Hum. Gene Ther., 7:1503-14 (1996)), bone osteocalcin promoter (Stein et al., Mol. Biol. Rep., 24:185-96 (1997)); bone sialoprotein promoter (Chen et al., J. Bone Miner. Res., 11:654-64 (1996)), CD2 promoter (Hansal et al., J. Immunol., 161:1063-8 (1998); immunoglobulin heavy chain promoter; T cell receptor a-chain promoter, neuronal such as neuron-specific enolase (NSE) promoter (Andersen et al., Cell. Mol. Neurobiol., 13:503-15 (1993)), neurofilament light-chain gene promoter (Piccioli et al., Proc. Natl. Acad. Sci. USA, 88:5611-5 (1991)), and the neuron-specific vgf gene promoter (Piccioli et al., Neuron, 15:373-84 (1995)), and others. Additional exemplary promoter sequences are described, for example, in U.S. patent Ser. No. 10/300,146 (incorporated herein by reference in its entirety). In some embodiments, a tissue-specific regulatory element, e.g., a tissue-specific promoter, is selected from one known to be operably linked to a gene that is highly expressed in a given tissue, e.g., as measured by RNA-seq or protein expression data, or a combination thereof. Methods for analyzing tissue specificity by expression are taught in Fagerberg et al. Mol Cell Proteomics 13(2):397-406 (2014), which is incorporated herein by reference in its entirety.

In some embodiments, a vector described herein is a multicistronic expression construct. Multicistronic expression constructs include, for example, constructs harboring a first expression cassette, e.g. comprising a first promoter and a first encoding nucleic acid sequence, and a second expression cassette, e.g. comprising a second promoter and a second encoding nucleic acid sequence. Such multicistronic expression constructs may, in some instances, be particularly useful in the delivery of non-translated gene products, such as hairpin RNAs, together with a polypeptide, for example, a gene writer and gene writer template. In some embodiments, multicistronic expression constructs may exhibit reduced expression levels of one or more of the included transgenes, for example, because of promoter interference or the presence of incompatible nucleic acid elements in close proximity. If a multicistronic expression construct is part of a viral vector, the presence of a self-complementary nucleic acid sequence may, in some instances, interfere with the formation of structures necessary for viral reproduction or packaging.

In some embodiments, the sequence encodes an RNA with a hairpin. In some embodiments, the hairpin RNA is a guide RNA, a template RNA, shRNA, or a microRNA. In some embodiments, the first promoter is an RNA polymerase I promoter. In some embodiments, the first promoter is an RNA polymerase II promoter. In some embodiments, the second promoter is an RNA polymerase III promoter. In some embodiments, the second promoter is a U6 or H1 promoter. In some embodiments, the nucleic acid construct comprises the structure of AAV construct B1 or B2.

Without wishing to be bound by theory, multicistronic expression constructs may not achieve optimal expression levels as compared to expression systems containing only one cistron. One of the suggested causes of lower expression levels achieved with multicistronic expression constructs comprising two or more promoter elements is the phenomenon of promoter interference (see, e.g., Curtin J A, Dane A P, Swanson A, Alexander I E, Ginn S L. Bidirectional promoter interference between two widely used internal heterologous promoters in a late-generation lentiviral construct. Gene Ther. 2008 March; 15(5):384-90; and Martin-Duque P, Jezzard S, Kaftansis L, Vassaux G. Direct comparison of the insulatingproperties of two genetic elements in an adenoviral vector containing two different expression cassettes. Hum Gene Ther. 2004 October; 15(10):995-1002; both references incorporated herein by reference for disclosure of promoter interference phenomenon). In some embodiments, the problem of promoter interference may be overcome, e.g., by producing multicistronic expression constructs comprising only one promoter driving transcription of multiple encoding nucleic acid sequences separated by internal ribosomal entry sites. In some embodiments, single-promoter driven expression of multiple cistrons may result in uneven expression levels of the cistrons. In some embodiments, a promoter cannot efficiently be isolated and isolation elements may not be compatible with some gene transfer vectors, for example, some retroviral vectors.

### MicroRNAs

MicroRNAs (miRNAs) and other small interfering nucleic acids generally regulate gene expression via target RNA transcript cleavage/degradation or translational repression of the target messenger RNA (mRNA). miRNAs may, in some instances, be natively expressed, typically as final 19-25 non-translated RNA products. miRNAs generally exhibit their activity through sequence-specific interactions with the 3′ untranslated regions (UTR) of target mRNAs. These endogenously expressed miRNAs may form hairpin precursors that are subsequently processed into an miRNA duplex, and further into a mature single stranded miRNA molecule. This mature miRNA generally guides a multiprotein complex, miRISC, which identifies target 3′ UTR regions of target mRNAs based upon their complementarity to the mature miRNA. Useful transgene products may include, for example, miRNAs or miRNA binding sites that regulate the expression of a linked polypeptide. A non-limiting list of miRNA genes; the products of these genes and their homologues are useful as transgenes or as targets for small interfering nucleic acids (e.g., miRNA sponges, antisense oligonucleotides), e.g., in methods such as those listed in U.S. Ser. No. 10/300,146, 22:25-25:48, incorporated by reference. In some embodiments, one or more binding sites for one or more of the foregoing miRNAs are incorporated in a transgene, e.g., a transgene delivered by a rAAV vector, e.g., to inhibit the expression of the transgene in one or more tissues of an animal harboring the transgene. In some embodiments, a binding site may be selected to control the expression of a transgene in a tissue specific manner. For example, binding sites for the liver-specific miR-122 may be incorporated into a transgene to inhibit expression of that transgene in the liver. Additional exemplary miRNA sequences are described, for example, in U.S. patent Ser. No. 10/300,146 (incorporated herein by reference in its entirety). For liver-specific Gene Writing, however, overexpression of miR-122 may be utilized instead of using binding sites to effect miR-122-specific degradation. This miRNA is positively associated with hepatic differentiation and maturation, as well as enhanced expression of liver specific genes. Thus, in some embodiments, the coding sequence for miR-122 may be added to a component of a Gene Writing system to enhance a liver-directed therapy.

A miR inhibitor or miRNA inhibitor is generally an agent that blocks miRNA expression and/or processing. Examples of such agents include, but are not limited to, microRNA antagonists, microRNA specific antisense, microRNA sponges, and microRNA oligonucleotides (double-stranded, hairpin, short oligonucleotides) that inhibit miRNA interaction with a Drosha complex. MicroRNA inhibitors, e.g., miRNA sponges, can be expressed in cells from transgenes (e.g., as described in Ebert, M. S. Nature Methods, Epub Aug. 12, 2007; incorporated by reference herein in its entirety). In some embodiments, microRNA sponges, or other miR inhibitors, are used with the AAVs. microRNA sponges generally specifically inhibit miRNAs through a complementary heptameric seed sequence. In some embodiments, an entire family of miRNAs can be silenced using a single sponge sequence. Other methods for silencing miRNA function (derepression of miRNA targets) in cells will be apparent to one of ordinary skill in the art.

In some embodiments, a miRNA as described herein comprises a sequence listed in Table 4 of PCT Publication No. WO2020014209, incorporated herein by reference. Also incorporated herein by reference are the listing of exemplary miRNA sequences from WO2020014209.

In some embodiments, it is advantageous to silence one or more components of a Gene Writing system (e.g., mRNA encoding a Gene Writer polypeptide, a Gene Writer Template RNA, or a heterologous object sequence expressed from the genome after successful Gene Writing) in a portion of cells. In some embodiments, it is advantageous to restrict expression of a component of a Gene Writing system to select cell types within a tissue of interest.

For example, it is known that in a given tissue, e.g., liver, macrophages and immune cells, e.g., Kupffer cells in the liver, may engage in uptake of a delivery vehicle for one or more components of a Gene Writing system. In some embodiments, at least one binding site for at least one miRNA highly expressed in macrophages and immune cells, e.g., Kupffer cells, is included in at least one component of a Gene Writing system, e.g., nucleic acid encoding a Gene Writing polypeptide or a transgene. In some embodiments, a miRNA that targets the one or more binding sites is listed in a table referenced herein, e.g., miR-142, e.g., mature miRNA hsa-miR-142-5p or hsa-miR-142-3p.

In some embodiments, there may be a benefit to decreasing Gene Writer levels and/or Gene Writer activity in cells in which Gene Writer expression or overexpression of a transgene may have a toxic effect. For example, it has been shown that delivery of a transgene overexpression cassette to dorsal root ganglion neurons may result in toxicity of a gene therapy (see Hordeaux et al Sci Transl Med 12(569):eaba9188 (2020), incorporated herein by reference in its entirety). In some embodiments, at least one miRNA binding site may be incorporated into a nucleic acid component of a Gene Writing system to reduce expression of a system component in a neuron, e.g., a dorsal root ganglion neuron. In some embodiments, the at least one miRNA binding site incorporated into a nucleic acid component of a Gene Writing system to reduce expression of a system component in a neuron is a binding site of miR-182, e.g., mature miRNA hsa-miR-182-5p or hsa-miR-182-3p. In some embodiments, the at least one miRNA binding site incorporated into a nucleic acid component of a Gene Writing system to reduce expression of a system component in a neuron is a binding site of miR-183, e.g., mature miRNA hsa-miR-183-5p or hsa-miR-183-3p. In some embodiments, combinations of miRNA binding sites may be used to enhance the restriction of expression of one or more components of a Gene Writing system to a tissue or cell type of interest.

The table below provides exemplary miRNAs and corresponding expressing cells, e.g., a miRNA for which one can, in some embodiments, incorporate binding sites (complementary sequences) in the transgene or polypeptide nucleic acid, e.g., to decrease expression in that off-target cell.

TABLE 10
Exemplary miRNA from off-target cells and tissues

| Silenced | miRNA | Mature | |
|---|---|---|---|
| cell type | name | miRNA | miRNA sequence |
| Kupffer cells | miR-142 | hsa-miR- | cauaaaguagaaag |
| | | 142-5p | cacuacu |
| Kupffer cells | miR-142 | hsa-miR- | uguaguguuuccua |
| | | 142-3p | cuuuaugga |
| Dorsal root | miR-182 | hsa-miR- | uuuggcaaugguag |
| ganglion neurons | | 182-5p | aacucacacu |
| Dorsal root | miR-182 | hsa-miR- | ugguucuagacuug |
| ganglion neurons | | 182-3p | ccaacua |
| Dorsal root | miR-183 | hsa-miR- | uauggcacugguag |
| ganglion neurons | | 183-5p | aauucacu |
| Dorsal root | miR-183 | hsa-miR- | gugaauuaccgaag |
| ganglion neurons | | 183-3p | ggccauaa |
| Hepatocytes | miR-122 | hsa-miR- | uggagugugacaau |
| | | 122-5p | gguguuug |
| Hepatocytes | miR-122 | hsa-miR- | aacgccauuaucac |
| | | 122-3p | acuaaaua |

### 5′ UTR and 3′ UTR

In certain embodiments, a nucleic acid comprising an open reading frame encoding a Gene Writer polypeptide (e.g., as described herein) comprises a 5′ UTR and/or a 3′ UTR. In embodiments, a 5′ UTR and 3′ UTR for protein expression, e.g., mRNA (or DNA encoding the RNA) for a Gene Writer polypeptide or heterologous object sequence, comprise optimized expression sequences. In some embodiments, the 5′ UTR comprises GGGAAAUAAGAGAGAAAAGAAGAGUAAGAAGAAAUAUAAGAGCCACC and/or the 3′ UTR comprising UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA, e.g., as described in Richner et al. Cell 168(6): P1114-1125 (2017), the sequences of which are incorporated herein by reference.

In some embodiments, an open reading frame of a Gene Writer system, e.g., an ORF of an mRNA (or DNA encoding an mRNA) encoding a Gene Writer polypeptide or one or more ORFs of an mRNA (or DNA encoding an mRNA) of a heterologous object sequence, is flanked by a 5′ and/or 3′ untranslated region (UTR) that enhances the expression thereof. In some embodiments, the 5′ UTR of an mRNA component (or transcript produced from a DNA component) of the system comprises the sequence 5′-GGGAAAUAAGAGAGAAAAGAAGAGUAAGAAGAAAUAUAAGAGCCACC-3′. In some embodiments, the 3′ UTR of an mRNA component (or transcript produced from a DNA component) of the system comprises the sequence 5′-UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA-3′. This combination of 5′ UTR and 3′ UTR has been shown to result in desirable expression of an operably linked ORF by Richner et al. Cell 168(6): P1114-1125 (2017), the teachings and sequences of which are incorporated herein by reference. In some embodiments, a system described herein comprises a DNA encoding a transcript, wherein the DNA comprises the corresponding 5′ UTR and 3′ UTR sequences, with T substituting for U in the above-listed sequence). In some embodiments, a DNA vector used to produce an RNA component of the system further comprises a promoter upstream of the 5′ UTR for initiating in vitro transcription, e.g., a T7, T3, or SP6 promoter. The 5′ UTR above begins with GGG, which is a suitable start for optimizing transcription using T7 RNA polymerase. For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

### Viral Vectors and Components Thereof

Viruses are a useful source of delivery vehicles for the systems described herein, in addition to a source of relevant enzymes or domains as described herein, e.g., as sources of recombinases and DNA binding domains used herein, e.g., Cre recombinase, lambda integrase, or the DNA binding domains from AAV Rep proteins. Some enzymes may have multiple activities. In some embodiments, the virus used as a Gene Writer delivery system or a source of components thereof may be selected from a group as described by Baltimore BacteriolRev 35(3):235-241 (1971).

In some embodiments, the virus is selected from a Group I virus, e.g., is a DNA virus and packages dsDNA into virions. In some embodiments, the Group I virus is selected from, e.g., Adenoviruses, Herpesviruses, Poxviruses.

In some embodiments, the virus is selected from a Group II virus, e.g., is a DNA virus and packages ssDNA into virions. In some embodiments, the Group II virus is selected from, e.g., Parvoviruses. In some embodiments, the parvovirus is a dependoparvovirus, e.g., an adeno-associated virus (AAV).

In some embodiments, the virus is selected from a Group III virus, e.g., is an RNA virus and packages dsRNA into virions. In some embodiments, the Group III virus is selected from, e.g., Reoviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group IV virus, e.g., is an RNA virus and packages ssRNA(+) into virions. In some embodiments, the Group IV virus is selected from, e.g., Coronaviruses, Picornaviruses, Togaviruses. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps.

In some embodiments, the virus is selected from a Group V virus, e.g., is an RNA virus and packages ssRNA(−) into virions. In some embodiments, the Group V virus is selected from, e.g., Orthomyxoviruses, Rhabdoviruses. In some embodiments, an RNA virus with an ssRNA(−) genome also carries an enzyme inside the virion that is transduced to host cells with the viral genome, e.g., an RNA-dependent RNA polymerase, capable of copying the ssRNA(−) into ssRNA(+) that can be translated directly by the host.

In some embodiments, the virus is selected from a Group VI virus, e.g., is a retrovirus and packages ssRNA(+) into virions. In some embodiments, the Group VI virus is selected from, e.g., Retroviruses. In some embodiments, the retrovirus is a lentivirus, e.g., HIV-1, HIV-2, SIV, BIV. In some embodiments, the retrovirus is a spumavirus, e.g., a foamy virus, e.g., HFV, SFV, BFV. In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host.

In further embodiments, the virus is selected from a Group VI virus, e.g., is a retrovirus and packages ssRNA(+) into virions. In some embodiments, the Group VI virus is selected from, e.g., Retroviruses. In some embodiments, the retrovirus is a lentivirus, e.g., HIV-1 or HIV-2. In some embodiments, the retrovirus is a spumavirus, e.g., a foamy virus, e.g., human foamy virus (HFV). In some embodiments, the retrovirus is a deltaretrovirus, e.g., Human T-lymphotropic virus type 1 (HTLV-1) or HTLV-2. In some embodiments, the retrovirus is a gammaretrovirus, e.g., murine leukemia virus (MLV). In some embodiments, the ssRNA(+) contained in such virions is a coding molecule able to serve directly as mRNA upon transduction into a host cell, e.g., can be directly translated into protein upon transduction into a host cell without requiring any intervening nucleic acid replication or polymerization steps. In some embodiments, the ssRNA(+) is first reverse transcribed and copied to generate a dsDNA genome intermediate from which mRNA can be transcribed in the host cell. In some embodiments, an RNA virus with an ssRNA(+) genome also carries an enzyme inside the virion that is transduced to host cells with the viral genome, e.g., an RNA-dependent DNA polymerase, capable of copying the ssRNA(+) into dsDNA that can be transcribed into mRNA and translated by the host. In some embodiments, the reverse transcriptase domain is substituted with a heterologous reverse transcriptase domain, including, without limitation, a reverse transcriptase domain from a heterologous retrovirus, RNA virus, non-LTR retrotransposon, group II intron, diversity-generating element, retron, telomerase, retroplasmid, or an engineered polymerase, e.g., RTX (Ellefson et al Science 2016). The diversity of reverse transcriptases has been described in, but not limited to, those used by prokaryotes (Zimmerly et al. Microbiol 5 Spectr 3(2):MDNA3-0058-2014 (2015); Lampson B.C. (2007) Prokaryotic Reverse Transcriptases. In: Polaina J., MacCabe A.P. (eds) Industrial Enzymes. Springer, Dordrecht), viruses (Herschhorn et al. Cell Mol Life Sci 67(16):2717-2747 (2010); Menendez-Arias et al. Virus Res 234:153-176 (2017)), and mobile elements (Eickbush et al. Virus Res 134(1-2):221-234 (2008); Craig et al. Mobile DNA III 3^{rd} 10 Ed. DOI:10.1128/9781555819217 (2015)), each of which is incorporated herein by reference.

In some embodiments, the virus is selected from a Group VII virus, e.g., is a retrovirus and packages dsRNA into virions. In some embodiments, the Group VII virus is selected from, e.g., Hepadnaviruses. In some embodiments, one or both strands of the dsRNA contained in such virions is a coding molecule able to serve directly as mRNA upon transduction into a host cell, e.g., can be directly translated into protein upon transduction into a host cell without requiring any intervening nucleic acid replication or polymerization steps. In some embodiments, one or both strands of the dsRNA contained in such virions is first reverse transcribed and copied to generate a dsDNA genome intermediate from which mRNA can be transcribed in the host cell. In some embodiments, an RNA virus with a dsRNA genome also carries an enzyme inside the virion that is transduced to host cells with the viral genome, e.g., an RNA-dependent DNA polymerase, capable of copying the dsRNA into dsDNA that can be transcribed into mRNA and translated by the host.

In some embodiments, virions used to deliver nucleic acid in this invention may also carry enzymes involved in the process of Gene Writing. For example, a virion may contain a recombinase domain that is delivered into a host cell along with the nucleic acid. In some embodiments, a template nucleic acid may be associated with a Gene Writer polypeptide within a virion, such that both are co-delivered to a target cell upon transduction of the nucleic acid from the viral particle. In some embodiments, the nucleic acid in a virion may comprise DNA, e.g., linear ssDNA, linear dsDNA, circular ssDNA, circular dsDNA, minicircle DNA, dbDNA, ceDNA. In some embodiments, the nucleic acid in a virion may comprise RNA, e.g., linear ssRNA, linear dsRNA, circular ssRNA, circular dsRNA. In some embodiments, a viral genome may circularize upon transduction into a host cell, e.g., a linear ssRNA molecule may undergo a covalent linkage to form a circular ssRNA, a linear dsRNA molecule may undergo a covalent linkage to form a circular dsRNA or one or more circular ssRNA. In some embodiments, a viral genome may replicate by rolling circle replication in a host cell. In some embodiments, a viral genome may comprise a single nucleic acid molecule, e.g., comprise a non-segmented genome.

In some embodiments, a viral genome may comprise two or more nucleic acid molecules, e.g., comprise a segmented genome. In some embodiments, a nucleic acid in a virion may be associated with one or proteins. In some embodiments, one or more proteins in a virion may be delivered to a host cell upon transduction. In some embodiments, a natural virus may be adapted for nucleic acid delivery by the addition of virion packaging signals to the target nucleic acid, wherein a host cell is used to package the target nucleic acid containing the packaging signals.

In some embodiments, a virion used as a delivery vehicle may comprise a commensal human virus. In some embodiments, a virion used as a delivery vehicle may comprise an anellovirus, the use of which is described in WO2018232017A1, which is incorporated herein by reference in its entirety.

### Lentiviral Components and Helper Cells

In some embodiments, the retroviral nucleic acid comprises one or more of (e.g., all of): a 5′ promoter (e.g., to control expression of the entire packaged RNA), a 5′ LTR (e.g., that includes R (polyadenylation tail signal) and/or U5 which includes a primer activation signal), a primer binding site, a psi packaging signal, a RRE element for nuclear export, a promoter directly upstream of the transgene to control transgene expression, a transgene (or other exogenous agent element), a polypurine tract, and a 3′ LTR (e.g., that includes a mutated U3, a R, and U5). In some embodiments, the retroviral nucleic acid further comprises one or more of a cPPT and a WPRE.

A retrovirus typically replicates by reverse transcription of its genomic RNA into a linear double-stranded DNA copy and subsequently covalently integrates its genomic DNA into a host genome. Illustrative retroviruses suitable for use in particular embodiments, include, but are not limited to: Moloney murine leukemia virus (M-MuLV), Moloney murine sarcoma virus (MoMSV), Harvey murine sarcoma virus (HaMuSV), murine mammary tumor virus (MuMTV), gibbon ape leukemia virus (GaLV), feline leukemia virus (FLV), spumavirus, Friend murine leukemia virus, Murine Stem Cell Virus (MSCV) and Rous Sarcoma Virus (RSV)) and lentivirus.

In some embodiments the retrovirus is a Gammretrovirus. In some embodiments the retrovirus is an Epsilonretrovirus. In some embodiments the retrovirus is an Alpharetrovirus. In some embodiments the retrovirus is a Betaretrovirus. In some embodiments the retrovirus is a Deltaretrovirus. In some embodiments the retrovirus is a Lentivirus. In some embodiments the retrovirus is a Spumaretrovirus.

Illustrative lentiviruses include, but are not limited to: HIV (human immunodeficiency virus; including HIV type 1, and HIV type 2); visna-maedi virus (VMV) virus; the caprine arthritis-encephalitis virus (CAEV); equine infectious anemia virus (EIAV); feline immunodeficiency virus (FIV); bovine immune deficiency virus (BIV); and simian immunodeficiency virus (SIV). In some embodiments, HIV based vector backbones (i.e., HIV cis-acting sequence elements) are used.

In some embodiments, a vector herein is a nucleic acid molecule capable transferring or transporting another nucleic acid molecule. The transferred nucleic acid is generally linked to, e.g., inserted into, the vector nucleic acid molecule. A vector may include sequences that direct autonomous replication in a cell, or may include sequences sufficient to allow integration into host cell DNA. Useful vectors include, for example, plasmids (e.g., DNA plasmids or RNA plasmids), transposons, cosmids, bacterial artificial chromosomes, and viral vectors. Useful viral vectors include, e.g., replication defective retroviruses and lentiviruses.

A viral vector can comprise, e.g., a nucleic acid molecule (e.g., a transfer plasmid) that includes virus-derived nucleic acid elements that typically facilitate transfer of the nucleic acid molecule or integration into the genome of a cell or to a viral particle that mediates nucleic acid transfer. Viral particles will typically include various viral components and sometimes also host cell components in addition to nucleic acid(s). A viral vector can comprise, e.g., a virus or viral particle capable of transferring a nucleic acid into a cell, or to the transferred nucleic acid (e.g., as naked DNA). Viral vectors and transfer plasmids can comprise structural and/or functional genetic elements that are primarily derived from a virus. A retroviral vector can comprise a viral vector or plasmid containing structural and functional genetic elements, or portions thereof, that are primarily derived from a retrovirus. A lentiviral vector can comprise a viral vector or plasmid containing structural and functional genetic elements, or portions thereof, including LTRs that are primarily derived from a lentivirus.

In embodiments, a lentiviral vector (e.g., lentiviral expression vector) may comprise a lentiviral transfer plasmid (e.g., as naked DNA) or an infectious lentiviral particle. With respect to elements such as cloning sites, promoters, regulatory elements, heterologous nucleic acids, etc., it is to be understood that the sequences of these elements can be present in RNA form in lentiviral particles and can be present in DNA form in DNA plasmids.

In some vectors described herein, at least part of one or more protein coding regions that contribute to or are essential for replication may be absent compared to the corresponding wild-type virus. This makes the viral vector replication-defective. In some embodiments, the vector is capable of transducing a target non-dividing host cell and/or integrating its genome into a host genome.

The structure of a wild-type retrovirus genome often comprises a 5′ long terminal repeat (LTR) and a 3′ LTR, between or within which are located a packaging signal to enable the genome to be packaged, a primer binding site, integration sites to enable integration into a host cell genome and gag, pol and env genes encoding the packaging components which promote the assembly of viral particles. More complex retroviruses have additional features, such as rev and RRE sequences in HIV, which enable the efficient export of RNA transcripts of the integrated provirus from the nucleus to the cytoplasm of an infected target cell. In the provirus, the viral genes are flanked at both ends by regions called long terminal repeats (LTRs). The LTRs are involved in proviral integration and transcription. LTRs also serve as enhancer-promoter sequences and can control the expression of the viral genes. Encapsidation of the retroviral RNAs occurs by virtue of a psi sequence located at the 5′ end of the viral genome.

The LTRs themselves are typically similar (e.g., identical) sequences that can be divided into three elements, which are called U3, R and U5. U3 is derived from the sequence unique to the 3′ end of the RNA. R is derived from a sequence repeated at both ends of the RNA and U5 is derived from the sequence unique to the 5′ end of the RNA. The sizes of the three elements can vary considerably among different retroviruses. For the viral genome, the site of transcription initiation is typically at the boundary between U3 and R in one LTR and the site of poly (A) addition (termination) is at the boundary between R and U5 in the other LTR. U3 contains most of the transcriptional control elements of the provirus, which include the promoter and multiple enhancer sequences responsive to cellular and in some cases, viral transcriptional activator proteins. Some retroviruses comprise any one or more of the following genes that code for proteins that are involved in the regulation of gene expression: tot, rev, tax and rex.

With regard to the structural genes gag, pol and env themselves, gag encodes the internal structural protein of the virus. Gag protein is proteolytically processed into the mature proteins MA (matrix), CA (capsid) and NC (nucleocapsid). The pol gene encodes the reverse transcriptase (RT), which contains DNA polymerase, associated RNase H and integrase (IN), which mediate replication of the genome. The env gene encodes the surface (SU) glycoprotein and the transmembrane (TM) protein of the virion, which form a complex that interacts specifically with cellular receptor proteins. This interaction promotes infection, e.g., by fusion of the viral membrane with the cell membrane.

In a replication-defective retroviral vector genome, gag and/or pol may be absent or not functional. The R regions at both ends of the RNA are typically repeated sequences. U5 and U3 represent unique sequences at the 5′ and 3′ ends of the RNA genome respectively.

Retroviruses may also contain additional genes which code for proteins other than gag, pol and env. Examples of additional genes include (in HIV), one or more of vif, vpr, vpx, vpu, tat, rev and nef. EIAV has (amongst others) the additional gene S2. Proteins encoded by additional genes serve various functions, some of which may be duplicative of a function provided by a cellular protein. In EIAV, for example, tat acts as a transcriptional activator of the viral LTR (Derse and Newbold 1993 Virology 194:530-6; Maury et al. 1994 Virology 200:632-42). It binds to a stable, stem-loop RNA secondary structure referred to as TAR. Rev regulates and co-ordinates the expression of viral genes through rev-response elements (RRE) (Martarano et al. 1994 J. Virol. 68:3102-11). The mechanisms of action of these two proteins are thought to be broadly similar to the analogous mechanisms in the primate viruses. In addition, an EIAV protein, Ttm, has been identified that is encoded by the first exon of tat spliced to the env coding sequence at the start of the transmembrane protein. In addition to protease, reverse transcriptase and integrase, non-primate lentiviruses contain a fourth pol gene product which codes for a dUTPase. This may play a role in the ability of these lentiviruses to infect certain non-dividing or slowly dividing cell types.

In embodiments, a recombinant lentiviral vector (RLV) is a vector with sufficient retroviral genetic information to allow packaging of an RNA genome, in the presence of packaging components, into a viral particle capable of infecting a target cell. Infection of the target cell can comprise reverse transcription and integration into the target cell genome. The RLV typically carries non-viral coding sequences which are to be delivered by the vector to the target cell. In embodiments, an RLV is incapable of independent replication to produce infectious retroviral particles within the target cell. In some embodiments, the RLV lacks a functional gag and/or pol gene and/or other genes involved in replication. The vector may be configured as a split-intron vector, e.g., as described in PCT patent application WO 99/15683, which is herein incorporated by reference in its entirety.

In some embodiments, the lentiviral vector comprises a minimal viral genome, e.g., the viral vector has been manipulated so as to remove the non-essential elements and to retain the essential elements in order to provide the required functionality to infect, transduce and deliver a nucleotide sequence of interest to a target host cell, e.g., as described in WO 98/17815, which is herein incorporated by reference in its entirety.

A minimal lentiviral genome may comprise, e.g., (5′)R-U5-one or more first nucleotide sequences-U3-R(3′) However, the plasmid vector used to produce the lentiviral genome within a source cell can also include transcriptional regulatory control sequences operably linked to the lentiviral genome to direct transcription of the genome in a source cell. These regulatory sequences may comprise the natural sequences associated with the transcribed retroviral sequence, e.g., the 5′ U3 region, or they may comprise a heterologous promoter such as another viral promoter, for example the CMV promoter. Some lentiviral genomes comprise additional sequences to promote efficient virus production. For example, in the case of HIV, rev and RRE sequences may be included. Alternatively or combination, codon optimization may be used, e.g., the gene encoding the exogenous agent may be codon optimized, e.g., as described in WO 01/79518, which is herein incorporated by reference in its entirety. Alternative sequences which perform a similar or the same function as the rev/RRE system may also be used. For example, a functional analogue of the rev/RRE system is found in the Mason Pfizer monkey virus. This is known as CTE and comprises an RRE-type sequence in the genome which is believed to interact with a factor in the infected cell. The cellular factor can be thought of as a rev analogue. Thus, CTE may be used as an alternative to the rev/RRE system. In addition, the Rex protein of HTLV-I can functionally replace the Rev protein of HIV-I. Rev and Rex have similar effects to IRE-BP.

In some embodiments, a retroviral nucleic acid (e.g., a lentiviral nucleic acid, e.g., a primate or non-primate lentiviral nucleic acid) (1) comprises a deleted gag gene wherein the deletion in gag removes one or more nucleotides downstream of about nucleotide 350 or 354 of the gag coding sequence; (2) has one or more accessory genes absent from the retroviral nucleic acid; (3) lacks the tat gene but includes the leader sequence between the end of the 5′ LTR and the ATG of gag; and (4) combinations of (1), (2) and (3). In an embodiment the lentiviral vector comprises all of features (1) and (2) and (3). This strategy is described in more detail in WO 99/32646, which is herein incorporated by reference in its entirety.

In some embodiments, a primate lentivirus minimal system requires none of the HIV/SIV additional genes vif, vpr, vpx, vpu, tat, rev and nef for either vector production or for transduction of dividing and non-dividing cells. In some embodiments, an EIAV minimal vector system does not require S2 for either vector production or for transduction of dividing and non dividing cells.

The deletion of additional genes may permit vectors to be produced without the genes associated with disease in lentiviral (e.g. HIV) infections. In particular, tat is associated with disease. Secondly, the deletion of additional genes permits the vector to package more heterologous DNA. Thirdly, genes whose function is unknown, such as S2, may be omitted, thus reducing the risk of causing undesired effects. Examples of minimal lentiviral vectors are disclosed in WO 99/32646 and in WO 98/17815.

In some embodiments, the retroviral nucleic acid is devoid of at least tat and S2 (if it is an EIAV vector system), and possibly also vif, vpr, vpx, vpu and nef. In some embodiments, the retroviral nucleic acid is also devoid of rev, RRE, or both. In some embodiments the retroviral nucleic acid comprises vpx. The Vpx polypeptide binds to and induces the degradation of the SAMHD1 restriction factor, which degrades free dNTPs in the cytoplasm. Thus, the concentration of free dNTPs in the cytoplasm increases as Vpx degrades SAMIID1 and reverse transcription activity is increased, thus facilitating reverse transcription of the retroviral genome and integration into the target cell genome.

Different cells differ in their usage of particular codons. This codon bias corresponds to a bias in the relative abundance of particular tRNAs in the cell type. By altering the codons in the sequence so that they are tailored to match with the relative abundance of corresponding tRNAs, it is possible to increase expression. By the same token, it is possible to decrease expression by deliberately choosing codons for which the corresponding tRNAs are known to be rare in the particular cell type. Thus, an additional degree of translational control is available. An additional description of codon optimization is found, e.g., in WO 99/41397, which is herein incorporated by reference in its entirety.

Many viruses, including HIV and other lentiviruses, use a large number of rare codons and by changing these to correspond to commonly used mammalian codons, increased expression of the packaging components in mammalian producer cells can be achieved.

Codon optimization has a number of other advantages. By virtue of alterations in their sequences, the nucleotide sequences encoding the packaging components may have RNA instability sequences (INS) reduced or eliminated from them. At the same time, the amino acid sequence coding sequence for the packaging components is retained so that the viral components encoded by the sequences remain the same, or at least sufficiently similar that the function of the packaging components is not compromised. In some embodiments, codon optimization also overcomes the Rev/RRE requirement for export, rendering optimized sequences Rev independent. In some embodiments, codon optimization also reduces homologous recombination between different constructs within the vector system (for example between the regions of overlap in the gag-pol and env open reading frames). In some embodiments, codon optimization leads to an increase in viral titer and/or improved safety.

In some embodiments, only codons relating to INS are codon optimized. In other embodiments, the sequences are codon optimized in their entirety, with the exception of the sequence encompassing the frameshift site of gag-pol.

The gag-pol gene comprises two overlapping reading frames encoding the gag-pol proteins. The expression of both proteins depends on a frameshift during translation. This frameshift occurs as a result of ribosome “slippage” during translation. This slippage is thought to be caused at least in part by ribosome-stalling RNA secondary structures. Such secondary structures exist downstream of the frameshift site in the gag-pol gene. For HIV, the region of overlap extends from nucleotide 1222 downstream of the beginning of gag (wherein nucleotide 1 is the A of the gag ATG) to the end of gag (nt 1503). Consequently, a 281 bp fragment spanning the frameshift site and the overlapping region of the two reading frames is preferably not codon optimized. In some embodiments, retaining this fragment will enable more efficient expression of the gag-pol proteins. For EIAV, the beginning of the overlap is at nt 1262 (where nucleotide 1 is the A of the gag ATG). The end of the overlap is at nt 1461. In order to ensure that the frameshift site and the gag-pol overlap are preserved, the wild type sequence may be retained from nt 1156 to 1465.

Derivations from optimal codon usage may be made, for example, in order to accommodate convenient restriction sites, and conservative amino acid changes may be introduced into the gag-pol proteins.

In some embodiments, codon optimization is based on codons with poor codon usage in mammalian systems. The third and sometimes the second and third base may be changed.

Due to the degenerate nature of the genetic code, it will be appreciated that numerous gag-pol sequences can be achieved by a skilled worker. Also, there are many retroviral variants described which can be used as a starting point for generating a codon optimized gag-pol sequence. Lentiviral genomes can be quite variable. For example there are many quasi-species of HIV-I which are still functional. This is also the case for EIAV. These variants may be used to enhance particular parts of the transduction process. Examples of HIV-I variants may be found in the HIV databases maintained by Los Alamos National Laboratory. Details of EIAV clones may be found at the NCBI database maintained by the National Institutes of Health.

The strategy for codon optimized gag-pol sequences can be used in relation to any retrovirus, e.g., EIAV, FIV, BIV, CAEV, VMR, SIV, HIV-I and HIV-2. In addition this method could be used to increase expression of genes from HTLV-I, HTLV-2, HFV, HSRV, MLV and other retroviruses.

As described above, the packaging components for a retroviral vector can include expression products of gag, pol and env genes. In addition, packaging can utilize a short sequence of 4 stem loops followed by a partial sequence from gag and env as a packaging signal. Thus, inclusion of a deleted gag sequence in the retroviral vector genome (in addition to the full gag sequence on the packaging construct) can be used. In embodiments, the retroviral vector comprises a packaging signal that comprises about 40, or from 255 to 360 nucleotides of gag. In some embodiments, the retroviral vector includes a gag sequence which comprises one or more deletions, e.g., the gag sequence comprises about 360 nucleotides derivable from the N-terminus.

The retroviral vector, helper cell, helper virus, or helper plasmid may comprise retroviral structural and accessory proteins, for example gag, pol, env, tat, rev, vif, vpr, vpu, vpx, or nef proteins or other retroviral proteins. In some embodiments the retroviral proteins are derived from the same retrovirus. In some embodiments the retroviral proteins are derived from more than one retrovirus, e.g. 2, 3, 4, or more retroviruses.

The gag and pol coding sequences are generally organized as the Gag-Pol Precursor in native lentivirus. The gag sequence codes for a 55-kD Gag precursor protein, also called p55.

The p55 is cleaved by the virally encoded protease4 (a product of the pol gene) during the process of maturation into four smaller proteins designated MA (matrix [pl7]), CA (capsid

[p24]), NC (nucleocapsid [p9]), and p6. The pol precursor protein is cleaved away from Gag by a virally encoded protease, and further digested to separate the protease (plO), RT (p50), RNase H (p15), and integrase (p31) activities.

Native Gag-Pol sequences can be utilized in a helper vector (e.g., helper plasmid or helper virus), or modifications can be made. These modifications include, chimeric Gag-Pol, where the Gag and Pol sequences are obtained from different viruses (e.g., different species, subspecies, strains, clades, etc.), and/or where the sequences have been modified to improve transcription and/or translation, and/or reduce recombination.

In various examples, the retroviral nucleic acid includes a polynucleotide encoding a 150-250 (e.g., 168) nucleotide portion of a gag protein that (i) includes a mutated INS1 inhibitory sequence that reduces restriction of nuclear export of RNA relative to wild-type INS 1, (ii) contains two nucleotide insertion that results in frame shift and premature termination, and/or (iii) does not include INS2, INS3, and INS4 inhibitory sequences of gag. In some embodiments, a vector described herein is a hybrid vector that comprises both retroviral (e.g., lentiviral) sequences and non-lentiviral viral sequences. In some embodiments, a hybrid vector comprises retroviral e.g., lentiviral, sequences for reverse transcription, replication, integration and/or packaging.

According to certain specific embodiments, most or all of the viral vector backbone sequences are derived from a lentivirus, e.g., HIV-l. However, it is to be understood that many different sources of retroviral and/or lentiviral sequences can be used, or combined and numerous substitutions and alterations in certain of the lentiviral sequences may be accommodated without impairing the ability of a transfer vector to perform the functions described herein. A variety of lentiviral vectors are described in Naldini et ah, (1996a, 1996b, and 1998); Zufferey et ah, (1997); Dull et ah, 1998, U.S. Pat. Nos. 6,013,516; and 5,994,136, many of which may be adapted to produce a retroviral nucleic acid.

At each end of the provirus, long terminal repeats (LTRs) are typically found. An LTR typically comprises a domain located at the ends of retroviral nucleic acid which, in their natural sequence context, are direct repeats and contain U3, R and U5 regions. LTRs generally promote the expression of retroviral genes (e.g., promotion, initiation and polyadenylation of gene transcripts) and viral replication. The LTR can comprise numerous regulatory signals including transcriptional control elements, polyadenylation signals and sequences for replication and integration of the viral genome. The viral LTR is typically divided into three regions called U3, R and U5. The U3 region typically contains the enhancer and promoter elements. The U5 region is typically the sequence between the primer binding site and the R region and can contain the polyadenylation sequence. The R (repeat) region can be flanked by the U3 and U5 regions. The LTR is typically composed of U3, R and U5 regions and can appear at both the 5′ and 3′ ends of the viral genome. In some embodiments, adjacent to the 5′ LTR are sequences for reverse transcription of the genome (the tRNA primer binding site) and for efficient packaging of viral RNA into particles (the Psi site).

A packaging signal can comprise a sequence located within the retroviral genome which mediate insertion of the viral RNA into the viral capsid or particle, see e.g., Clever et ah, 1995. J. of Virology, Vol. 69, No. 4; pp. 2101-2109. Several retroviral vectors use a minimal packaging signal (a psi [Y] sequence) for encapsidation of the viral genome. In various embodiments, retroviral nucleic acids comprise modified 5′ LTR and/or 3′ LTRs. Either or both of the LTR may comprise one or more modifications including, but not limited to, one or more deletions, insertions, or substitutions. Modifications of the 3′ LTR are often made to improve the safety of lentiviral or retroviral systems by rendering viruses replication-defective, e.g., virus that is not capable of complete, effective replication such that infective virions are not produced (e.g., replication-defective lentiviral progeny).

In some embodiments, a vector is a self-inactivating (SIN) vector, e.g., replication-defective vector, e.g., retroviral or lentiviral vector, in which the right (3′) LTR enhancer-promoter region, known as the U3 region, has been modified (e.g., by deletion or substitution) to prevent viral transcription beyond the first round of viral replication. This is because the right (3′) LTR U3 region can be used as a template for the left (5′) LTR U3 region during viral replication and, thus, absence of the U3 enhancer-promoter inhibits viral replication. In embodiments, the 3′ LTR is modified such that the U5 region is removed, altered, or replaced, for example, with an exogenous poly(A) sequence The 3′ LTR, the 5′ LTR, or both 3′ and 5′ LTRs, may be modified LTRs.

In some embodiments, the U3 region of the 5′ LTR is replaced with a heterologous promoter to drive transcription of the viral genome during production of viral particles.

Examples of heterologous promoters which can be used include, for example, viral simian virus 40 (SV40) (e.g., early or late), cytomegalovirus (CMV) (e.g., immediate early), Moloney murine leukemia virus (MoMLV), Rous sarcoma virus (RSV), and herpes simplex virus (HSV) (thymidine kinase) promoters. In some embodiments, promoters are able to drive high levels of transcription in a Tat-independent manner. In certain embodiments, the heterologous promoter has additional advantages in controlling the manner in which the viral genome is transcribed. Lor example, the heterologous promoter can be inducible, such that transcription of all or part of the viral genome will occur only when the induction factors are present. Induction factors include, but are not limited to, one or more chemical compounds or the physiological conditions such as temperature or pH, in which the host cells are cultured.

In some embodiments, viral vectors comprise a TAR (trans-activation response) element, e.g., located in the R region of lentiviral (e.g., HIV) LTRs. This element interacts with the lentiviral trans-activator (tat) genetic element to enhance viral replication. However, this element is not required, e.g., in embodiments wherein the U3 region of the 5′ LTR is replaced by a heterologous promoter.

The R region, e.g., the region within retroviral LTRs beginning at the start of the capping group (i.e., the start of transcription) and ending immediately prior to the start of the poly A tract can be flanked by the U3 and U5 regions. The R region plays a role during reverse transcription in the transfer of nascent DNA from one end of the genome to the other.

The retroviral nucleic acid can also comprise a FLAP element, e.g., a nucleic acid whose sequence includes the central polypurine tract and central termination sequences (cPPT and CTS) of a retrovirus, e.g., HIV-1 or HIV-2. Suitable FLAP elements are described in U.S. Pat. No. 6,682,907 and in Zennou, et al., 2000, Cell, 101:173, which are herein incorporated by reference in their entireties. During HIV-1 reverse transcription, central initiation of the plus-strand DNA at the central polypurine tract (cPPT) and central termination at the central termination sequence (CTS) can lead to the formation of a three-stranded DNA structure: the HIV-1 central DNA flap. In some embodiments, the retroviral or lentiviral vector backbones comprise one or more FLAP elements upstream or downstream of the gene encoding the exogenous agent. For example, in some embodiments a transfer plasmid includes a FLAP element, e.g., a FLAP element derived or isolated from HIV-L

In embodiments, a retroviral or lentiviral nucleic acid comprises one or more export elements, e.g., a cis-acting post-transcriptional regulatory element which regulates the transport of an RNA transcript from the nucleus to the cytoplasm of a cell. Examples of RNA export elements include, but are not limited to, the human immunodeficiency virus (HIV) rev response element (RRE) (see e.g., Cullen et ah, 1991. J. Virol. 65:1053; and Cullen et ah, 1991. Cell 58: 423), and the hepatitis B virus post-transcriptional regulatory element (HPRE), which are herein incorporated by reference in their entireties. Generally, the RNA export element is placed within the 3′ UTR of a gene, and can be inserted as one or multiple copies.

In some embodiments, expression of heterologous sequences in viral vectors is increased by incorporating one or more of, e.g., all of, posttranscriptional regulatory elements,
polyadenylation sites, and transcription termination signals into the vectors. A variety of posttranscriptional regulatory elements can increase expression of a heterologous nucleic acid at the protein, e.g., woodchuck hepatitis virus posttranscriptional regulatory element (WPRE; Zufferey et al., 1999, J. Virol., 73:2886); the posttranscriptional regulatory element present in hepatitis B virus (HPRE) (Huang et al., Mol. Cell. Biol., 5:3864); and the like (Liu et al., 1995, Genes Dev., 9:1766), each of which is herein incorporated by reference in its entirety. In some embodiments, a retroviral nucleic acid described herein comprises a posttranscriptional regulatory element such as a WPRE or HPRE

In some embodiments, a retroviral nucleic acid described herein lacks or does not comprise a posttranscriptional regulatory element such as a WPRE or HPRE.

Elements directing the termination and polyadenylation of the heterologous nucleic acid transcripts may be included, e.g., to increases expression of the exogenous agent. Transcription termination signals may be found downstream of the polyadenylation signal. In some embodiments, vectors comprise a polyadenylation sequence 3′ of a polynucleotide encoding the exogenous agent. A polyA site may comprise a DNA sequence which directs both the termination and polyadenylation of the nascent RNA transcript by RNA polymerase II.

Polyadenylation sequences can promote mRNA stability by addition of a polyA tail to the 3′ end of the coding sequence and thus, contribute to increased translational efficiency. Illustrative examples of polyA signals that can be used in a retroviral nucleic acid, include AATAAA,
ATT AAA, AGTAAA, a bovine growth hormone polyA sequence (BGHpA), a rabbit b-globin polyA sequence (rPgpA), or another suitable heterologous or endogenous polyA sequence.

In various embodiments, the vectors comprise a promoter operably linked to a polynucleotide encoding an exogenous agent. The vectors may have one or more LTRs, wherein either LTR comprises one or more modifications, such as one or more nucleotide substitutions, additions, or deletions. The vectors may further comprise one of more accessory elements to increase transduction efficiency (e.g., a cPPT/FLAP), viral packaging (e.g., a Psi (Y) packaging signal, RRE), and/or other elements that increase exogenous gene expression (e.g., poly (A) sequences), and may optionally comprise a WPRE or HPRE.

In some embodiments, a lentiviral nucleic acid comprises one or more of, e.g., all of, e.g., from 5′ to 3′, a promoter (e.g., CMV), an R sequence (e.g., comprising TAR), a U5 sequence (e.g., for integration), a PBS sequence (e.g., for reverse transcription), a DIS sequence (e.g., for genome dimerization), a psi packaging signal, a partial gag sequence, an RRE sequence (e.g., for nuclear export), a cPPT sequence (e.g., for nuclear import), a promoter to drive expression of the exogenous agent, a gene encoding the exogenous agent, a WPRE sequence (e.g., for efficient transgene expression), a PPT sequence (e.g., for reverse transcription), an R sequence (e.g., for polyadenylation and termination), and a U5 signal (e.g., for integration).

### Vectors Engineered to Remove Splice Sites

Some lentiviral vectors integrate inside active genes and possess strong splicing and polyadenylation signals that could lead to the formation of aberrant and possibly truncated transcripts.

Mechanisms of proto-oncogene activation may involve the generation of chimeric transcripts originating from the interaction of promoter elements or splice sites contained in the genome of the insertional mutagen with the cellular transcriptional unit targeted by integration (Gabriel et al. 2009. Nat Med 15: 1431-1436; Bokhoven, et al. J Virol 83:283-29). Chimeric fusion transcripts comprising vector sequences and cellular mRNAs can be generated either by read-through transcription starting from vector sequences and proceeding into the flanking cellular genes, or vice versa.

In some embodiments, a lentiviral nucleic acid described herein comprises a lentiviral backbone in which at least two of the splice sites have been eliminated, e.g., to improve the safety profile of the lentiviral vector. Species of such splice sites and methods of identification are described in WO2012156839A2, all of which is included by reference.

### Retroviral Production Methods

Large scale viral particle production is often useful to achieve a desired viral titer. Viral particles can be produced by transfecting a transfer vector into a packaging cell line that comprises viral structural and/or accessory genes, e.g., gag, pol, env, tat, rev, vif, vpr, vpu, vpx, or nef genes or other retroviral genes.

In embodiments, the packaging vector is an expression vector or viral vector that lacks a packaging signal and comprises a polynucleotide encoding one, two, three, four or more viral structural and/or accessory genes. Typically, the packaging vectors are included in a packaging cell, and are introduced into the cell via transfection, transduction or infection. A retroviral, e.g., lentiviral, transfer vector can be introduced into a packaging cell line, via transfection, transduction or infection, to generate a source cell or cell line. The packaging vectors can be introduced into human cells or cell lines by standard methods including, e.g., calcium phosphate transfection, lipofection or electroporation. In some embodiments, the packaging vectors are introduced into the cells together with a dominant selectable marker, such as neomycin, hygromycin, puromycin, blastocidin, zeocin, thymidine kinase, DHFR, Gln synthetase or ADA, followed by selection in the presence of the appropriate drug and isolation of clones. A selectable marker gene can be linked physically to genes encoding by the packaging vector, e.g., by IRES or self cleaving viral peptides.

Packaging cell lines include cell lines that do not contain a packaging signal, but do stably or transiently express viral structural proteins and replication enzymes (e.g., gag, pol, and env) which can package viral particles. Any suitable cell line can be employed, e.g., mammalian cells, e.g., human cells. Suitable cell lines which can be used include, for example, CHO cells, BHK cells, MDCK cells, C3H 10T1/2 cells, FLY cells, Psi-2 cells, BOSC 23 cells, PA317 cells, WEHI cells, COS cells, BSC 1 cells, BSC 40 cells, BMT 10 cells, VERO cells, W138 cells, MRC5 cells, A549 cells, HT1080 cells, 293 cells, 293T cells, B-50 cells, 3T3 cells, NIH3T3 cells, HepG2 cells, Saos-2 cells, Huh7 cells, HeLa cells, W163 cells, 211 cells, and 211 A cells.

In embodiments, the packaging cells are 293 cells, 293T cells, or A549 cells.

A source cell line includes a cell line which is capable of producing recombinant retroviral particles, comprising a packaging cell line and a transfer vector construct comprising a packaging signal. Methods of preparing viral stock solutions are illustrated by, e.g., Y. Soneoka et al. (1995) Nucl. Acids Res. 23:628-633, and N. R. Landau et al. (1992) J. Virol. 66:5110-5113, which are incorporated herein by reference. Infectious virus particles may be collected from the packaging cells, e.g., by cell lysis, or collection of the supernatant of the cell culture. Optionally, the collected virus particles may be enriched or purified.

### Packaging Plasmids and Cell Lines

In some embodiments, the source cell comprises one or more plasmids coding for viral structural proteins and replication enzymes (e.g., gag, pol, and env) which can package viral particles. In some embodiments, the sequences coding for at least two of the gag, pol, and env precursors are on the same plasmid. In some embodiments, the sequences coding for the gag, pol, and env precursors are on different plasmids. In some embodiments, the sequences coding for the gag, pol, and env precursors have the same expression signal, e.g., promoter. In some embodiments, the sequences coding for the gag, pol, and env precursors have a different expression signal, e.g., different promoters. In some embodiments, expression of the gag, pol, and env precursors is inducible. In some embodiments, the plasmids coding for viral structural proteins and replication enzymes are transfected at the same time or at different times. In some embodiments, the plasmids coding for viral structural proteins and replication enzymes are transfected at the same time or at a different time from the packaging vector.

In some embodiments, the source cell line comprises one or more stably integrated viral structural genes. In some embodiments expression of the stably integrated viral structural genes is inducible.

In some embodiments, expression of the viral structural genes is regulated at the transcriptional level. In some embodiments, expression of the viral structural genes is regulated at the translational level. In some embodiments, expression of the viral structural genes is regulated at the post-translational level.

In some embodiments, expression of the viral structural genes is regulated by a tetracycline (Tet)-dependent system, in which a Tet-regulated transcriptional repressor (Tet-R) binds to DNA sequences included in a promoter and represses transcription by steric hindrance (Yao et al, 1998; Jones et al, 2005). Upon addition of doxycycline (dox), Tet-R is released, allowing transcription. Multiple other suitable transcriptional regulatory promoters, transcription factors, and small molecule inducers are suitable to regulate transcription of viral structural genes.

In some embodiments, the third-generation lentivirus components, human immunodeficiency virus type 1 (HIV) Rev, Gag/Pol, and an envelope under the control of Tet-regulated promoters and coupled with antibiotic resistance cassettes are separately integrated into the source cell genome. In some embodiments the source cell only has one copy of each of Rev, Gag/Pol, and an envelope protein integrated into the genome. In some embodiments a nucleic acid encoding the exogenous agent (e.g., a retroviral nucleic acid encoding the exogenous agent) is also integrated into the source cell genome. In some embodiments a nucleic acid encoding the exogenous agent is maintained episomally. In some embodiments a nucleic acid encoding the exogenous agent is transfected into the source cell that has stably integrated Rev, Gag/Pol, and an envelope protein in the genome. See, e.g., Milani et al. EMBO Molecular Medicine, 2017, which is herein incorporated by reference in its entirety.

In some embodiments, a retroviral nucleic acid described herein is unable to undergo reverse transcription. Such a nucleic acid, in embodiments, is able to transiently express an exogenous agent. The retrovirus or VLP, may comprise a disabled reverse transcriptase protein, or may not comprise a reverse transcriptase protein. In embodiments, the retroviral nucleic acid comprises a disabled primer binding site (PBS) and/or att site. In embodiments, one or more viral accessory genes, including rev, tat, vif, nef, vpr, vpu, vpx and S2 or functional equivalents thereof, are disabled or absent from the retroviral nucleic acid. In embodiments, one or more accessory genes selected from S2, rev and tat are disabled or absent from the retroviral nucleic acid.

### Strategies for Packaging a Retroviral Nucleic Acid

Typically, modern retroviral vector systems consist of viral genomes bearing cis-acting vector sequences for transcription, reverse-transcription, integration, translation and packaging of viral RNA into the viral particles, and (2) producer cells lines which express the trans-acting retroviral gene sequences (e.g., gag, pol and env) needed for production of virus particles. By separating the cis- and trans-acting vector sequences completely, the virus is unable to maintain replication for more than one cycle of infection. Generation of live virus can be avoided by a number of strategies, e.g., by minimizing the overlap between the cis- and trans-acting sequences to avoid recombination.

A viral vector particle which comprises a sequence that is devoid of or lacking viral RNA may be the result of removing or eliminating the viral RNA from the sequence. In one embodiment this may be achieved by using an endogenous packaging signal binding site on gag. Alternatively, the endogenous packaging signal binding site is on pol. In this embodiment, the RNA which is to be delivered will contain a cognate packaging signal. In another embodiment, a heterologous binding domain (which is heterologous to gag) located on the RNA to be delivered, and a cognate binding site located on gag or pol, can be used to ensure packaging of the RNA to be delivered. The heterologous sequence could be non-viral or it could be viral, in which case it may be derived from a different virus. The vector particles could be used to deliver therapeutic RNA, in which case functional integrase and/or reverse transcriptase is not required. These vector particles could also be used to deliver a therapeutic gene of interest, in which case pol is typically included.

In an embodiment, gag-pol are altered, and the packaging signal is replaced with a corresponding packaging signal. In this embodiment, the particle can package the RNA with the new packaging signal. The advantage of this approach is that it is possible to package an RNA sequence which is devoid of viral sequence for example, RNAi.

An alternative approach is to rely on over-expression of the RNA to be packaged. In one embodiment the RNA to be packaged is over-expressed in the absence of any RNA containing a packaging signal. This may result in a significant level of therapeutic RNA being packaged, and that this amount is sufficient to transduce a cell and have a biological effect.

In some embodiments, a polynucleotide comprises a nucleotide sequence encoding a viral gag protein or retroviral gag and pol proteins, wherein the gag protein or pol protein comprises a heterologous RNA binding domain capable of recognising a corresponding sequence in an RNA sequence to facilitate packaging of the RNA sequence into a viral vector particle.

In some embodiments, the heterologous RNA binding domain comprises an RNA binding domain derived from a bacteriophage coat protein, a Rev protein, a protein of the U 1 small nuclear ribonucleoprotein particle, a Nova protein, a TF111 A protein, a TIS 11 protein, a trp RNA-binding attenuation protein (TRAP) or a pseudouridine synthase.

In some embodiments, a method herein comprises detecting or confirming the absence of replication competent retrovirus. The methods may include assessing RNA levels of one or more target genes, such as viral genes, e.g. structural or packaging genes, from which gene products are expressed in certain cells infected with a replication-competent retrovirus, such as a gammaretro virus or lentivirus, but not present in a viral vector used to transduce cells with a heterologous nucleic acid and not, or not expected to be, present and/or expressed in cells not containing replication-competent retrovirus. Replication competent retrovirus may be determined to be present if RNA levels of the one or more target genes is higher than a reference value, which can be measured directly or indirectly, e.g. from a positive control sample containing the target gene. For further disclosure, see WO2018023094A1.

### Repression of a Gene Encoding an Exogenous Agent in a Source Cell

(Over-)expressed protein in the source cell may have an indirect or direct effect on vector virion assembly and/or infectivity. Incorporation of the exogenous agent into vector virions may also impact downstream processing of vector particles.

In some embodiments, a tissue-specific promoter is used to limit expression of the exogenous agent in source cells. In some embodiments, a heterologous translation control system is used in eukaryotic cell cultures to repress the translation of the exogenous agent in source cells. More specifically, the retroviral nucleic acid may comprise a binding site operably linked to the gene encoding the exogenous agent, wherein the binding site is capable of interacting with an RNA-binding protein such that translation of the exogenous agent is repressed or prevented in the source cell.

In some embodiments, the RNA-binding protein is tryptophan RNA-binding attenuation protein (TRAP), for example bacterial tryptophan RNA-binding attenuation protein. The use of an RNA-binding protein (e.g. the bacterial trp operon regulator protein, tryptophan RNA-binding attenuation protein, TRAP), and RNA targets to which it binds, will repress or prevent transgene translation within a source cell. This system is referred to as the Transgene Repression In vector Production cell system or TRIP system.

In embodiments, the placement of a binding site for an RNA binding protein (e.g., a TRAP-binding sequence, tbs) upstream of the NOI translation initiation codon allows specific repression of translation of mRNA derived from the internal expression cassette, while having no detrimental effect on production or stability of vector RNA. The number of nucleotides between the tbs and translation initiation codon of the gene encoding the exogenous agent may be varied from 0 to 12 nucleotides. The tbs may be placed downstream of an internal ribosome entry site (IRES) to repress translation of the gene encoding the exogenous agent in a multicistronic mRNA. Kill switch systems and amplification

In some embodiments, a polynucleotide or cell harboring the gene encoding the exogenous agent utilizes a suicide gene, e.g., an inducible suicide gene, to reduce the risk of direct toxicity and/or uncontrolled proliferation. In specific aspects, the suicide gene is not immunogenic to the host cell harboring the exogenous agent. Examples of suicide genes include caspase-9, caspase-8, or cytosine deaminase. Caspase-9 can be activated using a specific chemical inducer of dimerization (CID).

In certain embodiments, vectors comprise gene segments that cause target cells, e.g., immune effector cells, e.g., T cells, to be susceptible to negative selection in vivo. For instance, the transduced cell can be eliminated as a result of a change in the in vivo condition of the individual. The negative selectable phenotype may result from the insertion of a gene that confers sensitivity to an administered agent, for example, a compound. Negative selectable genes are known in the art, and include, inter alia the following: the Herpes simplex virus type I thymidine kinase (HSV-I TK) gene (Wigler et ah, Cell 11:223, 1977) which confers ganciclovir sensitivity; the cellular hypoxanthine phosphribosyltransferase (HPRT) gene, the cellular adenine phosphoribosyltransferase (APRT) gene, and bacterial cytosine deaminase, (Mullen et ah, Proc. Natl. Acad. Sci. USA. 89:33 (1992)).

In some embodiments, transduced cells, e.g., immune effector cells, such as T cells, comprise a polynucleotide further comprising a positive marker that enables the selection of cells of the negative selectable phenotype in vitro. The positive selectable marker may be a gene which, upon being introduced into the target cell, expresses a dominant phenotype permitting positive selection of cells carrying the gene. Genes of this type include, inter alia, hygromycin-B phosphotransferase gene (hph) which confers resistance to hygromycin B, the amino glycoside phosphotransferase gene (neo or aph) from Tn5 which codes for resistance to the antibiotic G418, the dihydrofolate reductase (DHFR) gene, the adenosine deaminase gene (ADA), and the multi-drug resistance (MDR) gene.

In some embodiments, the positive selectable marker and the negative selectable element are linked such that loss of the negative selectable element necessarily also is accompanied by loss of the positive selectable marker. For instance, the positive and negative selectable markers can be fused so that loss of one obligatorily leads to loss of the other. An example of a fused polynucleotide that yields as an expression product a polypeptide that confers both the desired positive and negative selection features described above is a hygromycin phosphotransferase thymidine kinase fusion gene (HyTK). Expression of this gene yields a polypeptide that confers hygromycin B resistance for positive selection in vitro, and ganciclovir sensitivity for negative selection in vivo. See Lupton S. D., et al, Mol. and Cell. Biology 1 1:3374-3378, 1991. In addition, in embodiments, the polynucleotides encoding the chimeric receptors are in retroviral vectors containing the fused gene, particularly those that confer hygromycin B resistance for positive selection in vitro, and ganciclovir sensitivity for negative selection in vivo, for example the HyTK retroviral vector described in Lupton, S. D. et al. (1991), supra. See also the publications of PCT U591/08442 and PCT/US94/05601, describing the use of bifunctional selectable fusion genes derived from fusing dominant positive selectable markers with negative selectable markers.

Suitable positive selectable markers can be derived from genes selected from the group consisting of hph, nco, and gpt, and suitable negative selectable markers can be derived from genes selected from the group consisting of cytosine deaminase, HSV-I TK, VZV TK, HPRT, APRT and gpt. Other suitable markers are bifunctional selectable fusion genes wherein the positive selectable marker is derived from hph or neo, and the negative selectable marker is derived from cytosine deaminase or a TK gene or selectable marker.

### Integration-Deficient Retroviral Systems

A Gene Writer system as described herein may include an integration-deficient retroviral system (e.g., derived from a retrovirus, such as a lentivirus). A retrovirus (e.g., lentivirus) can be engineered to be integration-deficient, such that integration of a template DNA (e.g., encoded by a template RNA as described herein), or a portion thereof, by the retroviral integrase is reduced, e.g., by at least 75% 80%, 85% 90%, 95%, 96%, 97%, 98%, 99%, or 100%, relative to an otherwise similar unmodified retrovirus. In some instances, a retrovirus is engineered to be non-integrating—e.g., such that the retroviral integrase, or equivalent thereof, cannot integrate the viral genome, or any substantial portion thereof, into the genome of the cell. In some instances, the viral genome, or a portion thereof, of an integration-deficient retroviral system can be integrated into the genome of the cell by another recombinase (e.g., a recombinase provided in cis with the integration-deficient retroviral system or a recombinase provided in trans). In some instances, the viral genome, or a portion thereof, is engineered to comprise a recognition sequence for the recombinase (e.g., an attP or attB site). In some instances, the genome of the cell comprises a cognate recognition sequence for the recombinase, such that the recombinase can recombine the recognition sequence in the viral genome (or a DNA molecule reverse transcribed therefrom, e.g., by the retroviral reverse transcriptase) with the cognate recombination sequence in the genome of the cell, thereby inducing integration of the viral genome sequence, or a portion thereof, into the genome of the cell.

In some instances, an integration-deficient system (e.g., a composition comprising a virus, a viral vector, e.g., a retroviral vector, e.g., a lentiviral vector) or a polypeptide thereof is substantially unable to integrate a template DNA into a target DNA (e.g., a genomic DNA, e.g., a chromosome or mitochondrial DNA). In some instances, an integration-deficient system comprises a mutation to an integrase (e.g., as described herein), a template RNA lacking a wild-type viral LTR sequence, or an inhibitor of the integrase. In some instances, an integration-deficient system results in a decrease in the level of integrated template DNA relative to an otherwise similar integration-competent viral system by at least 20%, 30%, 40%, 50%, 60%, 70%, 75%, 80%, 85%, 90%, 95%, 96%, 97%, 98%, 99%, 99.9%, or 100%. In some embodiments, an integration-deficient system can be supplemented with an exogenous integrase.

In some instances, making a retrovirus (e.g., lentivirus) integration-deficient promotes circularization of linear viral DNA after reverse transcriptase (thereby forming an episome) over integration of the linear viral DNA into the genome of the cell (e.g., by reducing or eliminating integration of the linear viral DNA into the genome of the cell). Linear retroviral DNA is generally formed by reverse transcription of a retroviral RNA genome (e.g., by a reverse transcriptase encoded by the pol gene of the retrovirus). A certain amount of circularization of the linear retroviral DNA can naturally occur, e.g., by mechanisms that include, without limitation, nonhomologous end-joining (NHEJ), homologous recombination via strand-invasion or single-strand annealing, closure of intermediate products of reverse transcription, auto-integration, and ligation if nicks present in intermediate products formed during reverse transcription (e.g., as described in Wanische et al. 2009, Mol. Therap. 17(8): 1316-1332; incorporated herein by reference in its entirety). Circularization can result in the formation of episomes containing two retroviral LTRs (e.g., when formed by NHEJ) or episosomes containing a single retroviral LTR (e.g., when formed by homologous recombination of ligation).

A retrovirus (e.g., lentivirus) can be rendered integration-deficient via several possible means. In some instances, the retroviral integrase (IN) is mutated such that its capacity to integrate the viral DNA (or portions thereof) into the genome is substantially reduced or abrogated. In some instances, the mutation of the retroviral integrase is a class I mutation, i.e., a mutation that specifically affects integrase function regarding DNA cleavage and/or integration. Class I mutations include, for example, mutations of the catalytic triad of the retroviral integrase. For example, class I mutations for HIV-1 include mutations of residues D64 (e.g., D64V), D116, and E152. In some instances, a class I mutation does not substantially alter the levels of viral DNA present. In some instances, the mutation comprises a non-class I mutation (e.g., a class II mutation).

In some embodiments, a retrovirus (e.g., lentivirus) is rendered integration-deficient by modifying one or both attachment sites at the ends of the viral genome, e.g., by mutating or deleting the attachment site(s). In some embodiments, a retrovirus (e.g., lentivirus) is rendered integration-deficient by the presence of one or more integrase inhibitors (e.g., small molecule integrase inhibitors). In embodiments, the integrase inhibitor is a strand-transfer inhibitor. In embodiments, the integrase inhibitor is raltegravir or elvitegravir.

In some embodiments, the recombinase used to integrate the viral genome sequence (or portion thereof) of an integration-deficient retroviral system into the genome of a cell is a serine recombinase (e.g., a serine integrase, e.g., as described herein). In some embodiments, the recombinase is packaged into the retroviral particle. In embodiments, the recombinase is fused to a retroviral protein (e.g., gag, pol, or a domain or portion thereof, e.g., a matrix protein). In some embodiments, the recombinase is provided in trans (e.g., by transfecting the cell with a DNA or RNA encoding the recombinase).

In some embodiments, successful integration of a sequence from the genome of an integration-deficient retroviral vector (e.g., as described herein) into the genome of a cell can be determined by unidirectional sequencing with primers capable of hybridizing to a sequence comprised in the sequence to be integrated. In some embodiments, the primers are capable of hybridizing to an LTR sequence comprised in the genome of the integration-deficient retroviral vector, or the reverse complement thereof. In some embodiments, successful integration of a sequence from the genome of an integration-deficient retroviral vector (e.g., as described herein) into the genome of a cell can be determined by ddPCR, e.g., targeting a specific sequence comprised in the genome of the integration-deficient retroviral vector, or the reverse complement thereof.

In some embodiments, an integration-deficient retroviral vector comprises one or more long terminal repeats (LTRs). In some embodiments, an integration-deficient retroviral vector comprises a 5′ LTR. In some embodiments, an integration-deficient retroviral vector comprises a 3′ LTR. In some embodiments, an integration-deficient retroviral vector comprises an LTR (e.g., a 5′ LTR) sequence comprising the nucleic acid sequence GGGTCTCTCTGGTTAGACCAGATCTGAGCCTGGGAGCTCTCTGGCTAACTAGGGAAC CCACTGCTTAAGCCTCAATAAAGCTTGCCTTGAGTGCTTCAAGTAGTGTGTGCCCGT CTGTTGTGTGACTCTGGTAACTAGAGATCCCTCAGACCCTTTTAGTCAGTGTGGAAA ATCTCTAGCA, or a nucleic acid sequence having at least 75%, 80%, 85%, 90%, 95%, 96%, 97%, 98%, or 99% sequence identity thereto. In some embodiments, an integration-deficient retroviral vector comprises an LTR (e.g., a 3′ LTR) sequence comprising the nucleic acid sequence GGGTCTCTCTGGTTAGACCAGATCTGAGCCTGGGAGCTCTCTGGCTAACTAGGGAAC CCACTGCTTAAGCCTCAATAAAGCTTGCCTTGAGTGCTTCAAGTAGTGTGTGCCCGT CTGTTGTGTGACTCTGGTAACTAGAGATCCCTCAGACCCTTTTAGTCAGTGTGGAAA ATCTCTAGCA, or a nucleic acid sequence having at least 75%, 80%, 85%, 90%, 95%, 96%, 97%, 98%, or 99% sequence identity thereto. In some embodiments, an integration-deficient retroviral vector comprises an LTR (e.g., a 5′ LTR) sequence comprising the nucleic acid sequence GGGTCTCTCTGGTTAGACCAGATCTGAGCCTGGGAGCTCTCTGGCTAACTAGGGAAC CCACTGCTTAAGCCTCAATAAAGCTTGCCTTGAGTGCTTCAAGTAGTGTGTGCCCGT CTGTTGTGTGACTCTGGTAACTAGAGATCCCTCAGACCCTTTTAGTCAGTGTGGAAA ATCTCTAGCA, or a nucleic acid sequence having at least 75%, 80%, 85%, 90%, 95%, 96%, 97%, 98%, or 99% sequence identity thereto. In some embodiments, an integration-deficient retroviral vector comprises an LTR (e.g., a 3′ LTR) sequence comprising the nucleic acid sequence TGGAAGGGCTAATTCACTCCCAACGAAGACAAGATCTGCTTTTTGCTTGTACTGGGT CTCTCTGGTTAGACCAGATCTGAGCCTGGGAGCTCTCTGGCTAACTAGGGAACCCAC TGCTTAAGCCTCAATAAAGCTTGCCTTGAGTGCTTCAAGTAGTGTGTGCCCGTCTGTT GTGTGACTCTGGTAACTAGAGATCCCTCAGACCCTTTTAGTCAGTGTGGAAAATCTC TAGCA, or a nucleic acid sequence having at least 75%, 80%, 85%, 90%, 95%, 96%, 97%, 98%, or 99% sequence identity thereto. In some embodiments, the LTR is a self-inactivating (SIN) LTR.

In some embodiments, an integration-deficient retroviral vector comprises a gag protein, a gag-pol protein, and/or an env protein, and/or functional fragments thereof (e.g., functional domains thereof). In some embodiments, an integration-deficient retroviral vector comprises an HIV-1 gag protein, a gag-pol protein, and/or an env protein, and/or functional fragments thereof (e.g., functional domains thereof). In some embodiments, an integration-deficient retroviral vector comprises one or more polypeptides having at least 75%, 80%, 85%, 90%, 95%, 96%, 97%, 98%, or 99% amino acid sequence identity to a gag protein, a gag-pol protein, and/or an env protein as listed in Table 11.

TABLE 11
Exemplary amino acid sequences of the
HIV-1 GAG, GAG-POL, and ENV polyproteins

| | NCBI | | |
|---|---|---|---|
| Protein | (RefSeq) | UniProt | Protein Sequence |
| GAG | NP_057850 | P04591 | MGARASVLSGGELDRWEKIRLRPGGKKKYKLKHIVWASRELERFA |
| | | | VNPGLLETSEGCRQILGQLQPSLQTGSEELRSLYNTVATLYCVHQRIE |
| | | | IKDTKEALDKIEEEQNKSKKKAQQAAADTGHSNQVSQNYPIVQNIQ |
| | | | GQMVHQAISPRTLNAWVKVVEEKAFSPEVIPMFSALSEGATPQDLN |
| | | | TMLNTVGGHQAAMQMLKETINEEAAEWDRVHPVHAGPIAPGQMR |
| | | | EPRGSDIAGTTSTLQEQIGWMTNNPPIPVGEIYKRWIILGLNKIVRMY |
| | | | SPTSILDIRQGPKEPFRDYVDRFYKTLRAEQASQEVKNWMTETLLVQ |
| | | | NANPDCKTILKALGPAATLEEMMTACQGVGGPGHKARVLAEAMSQ |
| | | | VTNSATIMMQRGNFRNQRKIVKCFNCGKEGHTARNCRAPRKKGCW |
| | | | KCGKEGHQMKDCTERQANFLGKIWPSYKGRPGNFLQSRPEPTAPPE |
| | | | ESFRSGVETTTPPQKQEPIDKELYPLTSLRSLFGNDPSSQ |
| GAG- | NP_057849 | P04585 | MGARASVLSGGELDRWEKIRLRPGGKKKYKLKHIVWASRELERFA |
| POL | | | VNPGLLETSEGCRQILGQLQPSLQTGSEELRSLYNTVATLYCVHQRIE |
| | | | IKDTKEALDKIEEEQNKSKKKAQQAAADTGHSNQVSQNYPIVQNIQ |
| | | | GQMVHQAISPRTLNAWVKVVEEKAFSPEVIPMFSALSEGATPQDLN |
| | | | TMLNTVGGHQAAMQMLKETINEEAAEWDRVHPVHAGPIAPGQMR |
| | | | EPRGSDIAGTTSTLQEQIGWMTNNPPIPVGEIYKRWIILGLNKIVRMY |
| | | | SPTSILDIRQGPKEPFRDYVDRFYKTLRAEQASQEVKNWMTETLLVQ |
| | | | NANPDCKTILKALGPAATLEEMMTACQGVGGPGHKARVLAEAMSQ |
| | | | VTNSATIMMQRGNFRNQRKIVKCFNCGKEGHTARNCRAPRKKGCW |
| | | | KCGKEGHQMKDCTERQANFLREDLAFLQGKAREFSSEQTRANSPTR |
| | | | RELQVWGRDNNSPSEAGADRQGTVSFNFPQVTLWQRPLVTIKIGGQ |
| | | | LKEALLDTGADDTVLEEMSLPGRWKPKMIGGIGGFIKVRQYDQILIEI |
| | | | CGHKAIGTVLVGPTPVNIIGRNLLTQIGCTLNFPISPIETVPVKLKPGM |
| | | | DGPKVKQWPLTEEKIKALVEICTEMEKEGKISKIGPENPYNTPVFAIK |
| | | | KKDSTKWRKLVDFRELNKRTQDFWEVQLGIPHPAGLKKKKSVTVL |
| | | | DVGDAYFSVPLDEDFRKYTAFTIPSINNETPGIRYQYNVLPQGWKGS |
| | | | PAIFQSSMTKILEPFRKQNPDIVIYQYMDDLYVGSDLEIGQHRTKIEEL |
| | | | RQHLLRWGLTTPDKKHQKEPPFLWMGYELHPDKWTVQPIVLPEKD |
| | | | SWTVNDIQKLVGKLNWASQIYPGIKVRQLCKLLRGTKALTEVIPLTE |
| | | | EAELELAENREILKEPVHGVYYDPSKDLIAEIQKQGQGQWTYQIYQE |
| | | | PFKNLKTGKYARMRGAHTNDVKQLTEAVQKITTESIVIWGKTPKFK |
| | | | LPIQKETWETWWTEYWQATWIPEWEFVNTPPLVKLWYQLEKEPIVG |
| | | | AETFYVDGAANRETKLGKAGYVTNRGRQKVVTLTDTTNQKTELQA |
| | | | IYLALQDSGLEVNIVTDSQYALGIIQAQPDQSESELVNQIIEQLIKKEK |
| | | | VYLAWVPAHKGIGGNEQVDKLVSAGIRKVLFLDGIDKAQDEHEKY |
| | | | HSNWRAMASDFNLPPVVAKEIVASCDKCQLKGEAMHGQVDCSPGI |
| | | | WQLDCTHLEGKVILVAVHVASGYIEAEVIPAETGQETAYFLLKLAGR |
| | | | WPVKTIHTDNGSNFTGATVRAACWWAGIKQEFGIPYNPQSQGVVES |
| | | | MNKELKKIIGQVRDQAEHLKTAVQMAVFIHNFKRKGGIGGYSAGER |
| | | | IVDIIATDIQTKELQKQITKIQNFRVYYRDSRNPLWKGPAKLLWKGE |
| | | | GAVVIQDNSDIKVVPRRKAKIIRDYGKQMAGDDCVASRQDED |
| ENV | NP_057856 | P04578 | MRVKEKYQHLWRWGWRWGTMLLGMLMICSATEKLWVTVYYGVP |
| | | | VWKEATTTLFCASDAKAYDTEVHNVWATHACVPTDPNPQEVVLVN |
| | | | VTENFNMWKNDMVEQMHEDIISLWDQSLKPCVKLTPLCVSLKCTD |
| | | | LKNDTNTNSSSGRMIMEKGEIKNCSFNISTSIRGKVQKEYAFFYKLDII |
| | | | PIDNDTTSYKLTSCNTSVITQACPKVSFEPIPIHYCAPAGFAILKCNNK |
| | | | TFNGTGPCTNVSTVQCTHGIRPVVSTQLLLNGSLAEEEVVIRSVNFTD |
| | | | NAKTIIVQLNTSVEINCTRPNNNTRKRIRIQRGPGRAFVTIGKIGNMR |
| | | | QAHCNISRAKWNNTLKQIASKLREQFGNNKTIIFKQSSGGDPEIVTHS |
| | | | FNCGGEFFYCNSTQLFNSTWFNSTWSTEGSNNTEGSDTITLPCRIKQII |
| | | | NMWQKVGKAMYAPPISGQIRCSSNITGLLLTRDGGNSNNESEIFRPG |
| | | | GGDMRDNWRSELYKYKVVKIEPLGVAPTKAKRRVVQREKRAVGIG |
| | | | ALFLGFLGAAGSTMGAASMTLTVQARQLLSGIVQQQNNLLRAIEAQ |
| | | | QHLLQLTVWGIKQLQARILAVERYLKDQQLLGIWGCSGKLICTTAVP |
| | | | WNASWSNKSLEQIWNHTTWMEWDREINNYTSLIHSLIEESQNQQEK |
| | | | NEQELLELDKWASLWNWFNITNWLWYIKLFIMIVGGLVGLRIVFAV |
| | | | LSIVNRVRQGYSPLSFQTHLPTPRGPDRPEGIEEEGGERDRDRSIRLV |
| | | | NGSLALIWDDLRSLCLFSYHRLRDLLLIVTRIVELLGRRGWEALKYW |
| | | | WNLLQYWSQELKNSAVSLLNATAIAVAEGTDRVIEVVQGACRAIRH |
| | | | IPRRIRQGLERILL |

In some embodiments, an integration-deficient retroviral vector comprises one or more polypeptides comprising a sequence at least 750, 800, 850, 900, 95q 96ec, 97e, 98%, or 9900 amino acid sequence identity to a gag-pol protein, matrix protein, capsid protein, nucleocapsid protein, pol protein, protease protein, RNaseH, reverse transcriptase (RT), integrase, peptidase A2 domain, reverse transcriptase domain, RnaseH domain, integrase catalytic domain, nuclear export signal, NLS, and/or tryptophan repeat motif, e.g., as listed in Table 12.

TABLE 12
Proteins comprising the HIV-1 GAG-POL
polyprotein and domains of particular importance

| Name | Type | Length | Start | Stop | Sequence |
|---|---|---|---|---|---|
| Gag-Pol | Protein | 1434 | 2 | 1435 | (See Table 11) |
| polyprotein | | | | | |
| Matrix | Protein | 131 | 2 | 132 | GARASVLSGGELDRWEKIRLRPGGKKKYKLKHIVW |
| protein p17 | | | | | ASRELERFAVNPGLLETSEGCRQILGQLQPSLQTGSE |
| | | | | | ELRSLYNTVATLYCVHQRIEIKDTKEALDKIEEEQN |
| | | | | | KSKKKAQQAAADTGHSNQVSQNY |
| Capsid | Protein | 231 | 133 | 363 | PIVQNIQGQMVHQAISPRTLNAWVKVVEEKAFSPEV |
| protein p24 | | | | | IPMFSALSEGATPQDLNTMLNTVGGHQAAMQMLK |
| | | | | | ETINEEAAEWDRVHPVHAGPIAPGQMREPRGSDIAG |
| | | | | | TTSTLQEQIGWMTNNPPIPVGEIYKRWIILGLNKIVR |
| | | | | | MYSPTSILDIRQGPKEPFRDYVDRFYKTLRAEQASQ |
| | | | | | EVKNWMTETLLVQNANPDCKTILKALGPAATLEEM |
| | | | | | MTACQGVGGPGHKARVL |
| Nucleocapsid | Protein | 55 | 378 | 432 | MQRGNFRNQRKIVKCFNCGKEGHTARNCRAPRKK |
| protein p7 | | | | | GCWKCGKEGHQMKDCTERQAN |
| p6-pol | Protein | 48 | 441 | 488 | LQGKAREFSSEQTRANSPTRRELQVWGRDNNSPSE |
| | | | | | AGADRQGTVSFNF |
| Protease | Protein | 99 | 489 | 587 | PQVTLWQRPLVTIKIGGQLKEALLDTGADDTVLEE |
| | | | | | MSLPGRWKPKMIGGIGGFIKVRQYDQILIEICGHKAI |
| | | | | | GTVLVGPTPVNIIGRNLLTQIGCTLNF |
| RT/RNaseH | Protein | 560 | 588 | 1147 | PISPIETVPVKLKPGMDGPKVKQWPLTEEKIKALVEI |
| (p66) | | | | | CTEMEKEGKISKIGPENPYNTPVFAIKKKDSTKWRK |
| | | | | | LVDFRELNKRTQDFWEVQLGIPHPAGLKKKKSVTV |
| | | | | | LDVGDAYFSVPLDEDFRKYTAFTIPSINNETPGIRYQ |
| | | | | | YNVLPQGWKGSPAIFQSSMTKILEPFRKQNPDIVIYQ |
| | | | | | YMDDLYVGSDLEIGQHRTKIEELRQHLLRWGLTTP |
| | | | | | DKKHQKEPPFLWMGYELHPDKWTVQPIVLPEKDS |
| | | | | | WTVNDIQKLVGKLNWASQIYPGIKVRQLCKLLRGT |
| | | | | | KALTEVIPLTEEAELELAENREILKEPVHGVYYDPSK |
| | | | | | DLIAEIQKQGQGQWTYQIYQEPFKNLKTGKYARMR |
| | | | | | GAHTNDVKQLTEAVQKITTESIVIWGKTPKFKLPIQ |
| | | | | | KETWETWWTEYWQATWIPEWEFVNTPPLVKLWY |
| | | | | | QLEKEPIVGAETFYVDGAANRETKLGKAGYVTNRG |
| | | | | | RQKVVTLTDTTNQKTELQAIYLALQDSGLEVNIVTD |
| | | | | | SQYALGIIQAQPDQSESELVNQIIEQLIKKEKVYLAW |
| | | | | | VPAHKGIGGNEQVDKLVSAGIRKVL |
| RT (p51) | Protein | 440 | 588 | 1027 | PISPIETVPVKLKPGMDGPKVKQWPLTEEKIKALVEI |
| | | | | | CTEMEKEGKISKIGPENPYNTPVFAIKKKDSTKWRK |
| | | | | | LVDFRELNKRTQDFWEVQLGIPHPAGLKKKKSVTV |
| | | | | | LDVGDAYFSVPLDEDFRKYTAFTIPSINNETPGIRYQ |
| | | | | | YNVLPQGWKGSPAIFQSSMTKILEPFRKQNPDIVIYQ |
| | | | | | YMDDLYVGSDLEIGQHRTKIEELRQHLLRWGLTTP |
| | | | | | DKKHQKEPPFLWMGYELHPDKWTVQPIVLPEKDS |
| | | | | | WTVNDIQKLVGKLNWASQIYPGIKVRQLCKLLRGT |
| | | | | | KALTEVIPLTEEAELELAENREILKEPVHGVYYDPSK |
| | | | | | DLIAEIQKQGQGQWTYQIYQEPFKNLKTGKYARMR |
| | | | | | GAHTNDVKQLTEAVQKITTESIVIWGKTPKFKLPIQ |
| | | | | | KETWETWWTEYWQATWIPEWEFVNTPPLVKLWY |
| | | | | | QLEKEPIVGAETF |
| RNase H | Protein | 120 | 1028 | 1147 | YVDGAANRETKLGKAGYVTNRGRQKVVTLTDTTN |
| | | | | | QKTELQAIYLALQDSGLEVNIVTDSQYALGIIQAQP |
| | | | | | DQSESELVNQIIEQLIKKEKVYLAWVPAHKGIGGNE |
| | | | | | QVDKLVSAGIRKVL |
| Integrase | Protein | 288 | 1148 | 1435 | FLDGIDKAQDEHEKYHSNWRAMASDFNLPPVVAK |
| | | | | | EIVASCDKCQLKGEAMHGQVDCSPGIWQLDCTHLE |
| | | | | | GKVILVAVHVASGYIEAEVIPAETGQETAYFLLKLA |
| | | | | | GRWPVKTIHTDNGSNFTGATVRAACWWAGIKQEF |
| | | | | | GIPYNPQSQGVVESMNKELKKIIGQVRDQAEHLKTA |
| | | | | | VQMAVFIHNFKRKGGIGGYSAGERIVDIIATDIQTKE |
| | | | | | LQKQITKIQNFRVYYRDSRNPLWKGPAKLLWKGEG |
| | | | | | AVVIQDNSDIKVVPRRKAKIIRDYGKQMAGDDCVA |
| | | | | | SRQDED |
| Peptidase A2 | Domain | 70 | 508 | 577 | KEALLDTGADDTVLEEMSLPGRWKPKMIGGIGGFI |
| domain | | | | | KVRQYDQILIEICGHKAIGTVLVGPTPVNIIGRNL |
| Reverse | Domain | 191 | 631 | 821 | EGKISKIGPENPYNTPVFAIKKKDSTKWRKLVDFRE |
| transcriptase | | | | | LNKRTQDFWEVQLGIPHPAGLKKKKSVTVLDVGDA |
| domain | | | | | YFSVPLDEDFRKYTAFTIPSINNETPGIRYQYNVLPQ |
| | | | | | GWKGSPAIFQSSMTKILEPFRKQNPDIVIYQYMDDL |
| | | | | | YVGSDLEIGQHRTKIEELRQHLLRWGLTTPDKKHQ |
| | | | | | KEPPFLWMGYEL |
| RNase H | Domain | 124 | 1021 | 1144 | IVGAETFYVDGAANRETKLGKAGYVTNRGRQKVV |
| domain | | | | | TLTDTTNQKTELQAIYLALQDSGLEVNIVTDSQYAL |
| | | | | | GIIQAQPDQSESELVNQIIEQLIKKEKVYLAWVPAHK |
| | | | | | GIGGNEQVDKLVSAGIR |
| Integrase | Domain | 151 | 1201 | 1351 | VDCSPGIWQLDCTHLEGKVILVAVHVASGYIEAEVI |
| catalytic | | | | | PAETGQETAYFLLKLAGRWPVKTIHTDNGSNFTGA |
| domain | | | | | TVRAACWWAGIKQEFGIPYNPQSQGVVESMNKELK |
| | | | | | KIIGQVRDQAEHLKTAVQMAVFIHNFKRKGGIGGY |
| | | | | | SAGERIVDII |
| Nuclear | Motif | 7 | 16 | 22 | WEKIRLR |
| export signal | | | | | |
| Nuclear | Motif | 7 | 26 | 32 | KKKYKLK |
| localization | | | | | |
| signal | | | | | |
| Tryptophan | Motif | 17 | 985 | 1001 | WETWWTEYWQATWIPEW |
| repeat motif | | | | | |

Retroviral and lentiviral nucleic acids are disclosed which are lacking or disabled in key proteins/sequences so as to prevent integration of the retroviral or lentiviral genome into the target cell genome. For instance, viral nucleic acids lacking each of the amino acids making up the highly conserved DDE motif (Engelman and Craigie (1992) J. Virol. 66:6361-6369; Johnson et al. (1986) Proc. Natl. Acad. Sci. USA 83:7648-7652; Khan et al. (1991) Nucleic Acids Res. 19:851-860) of retroviral integrase enables the production of integration defective retroviral nucleic acids.

For instance, in some embodiments, a retroviral nucleic acid herein comprises a lentiviral integrase comprising a mutation that causes said integrase to be unable to catalyze the integration of the viral genome into a cell genome. In some embodiments, said mutations are type I mutations which affect directly the integration, or type II mutations which trigger pleiotropic defects affecting virion morphogenesis and/or reverse transcription. Illustrative non-limitative examples of type I mutations are those mutations affecting any of the three residues that participate in the catalytic core domain of the integrase: DX39-58DX35E (D64, D116 and El 52 residues of the integrase of the HIV-1). In a particular embodiment, the mutation that causes said integrase to be unable to catalyze the integration of the viral genome into a cell genome is the substitution of one or more amino acid residues of the DDE motif of the catalytic core domain of the integrase, preferably the substitution of the first aspartic residue of said DEE motif by an asparagine residue. In some embodiment the retroviral vector does not comprise an integrase protein.

In some embodiments the retrovirus integrates into active transcription units. In some embodiments the retrovirus does not integrate near transcriptional start sites, the 5′ end of genes, or DNAse 1 cleavage sites. In some embodiments the retrovirus integration does not active proto oncogenes or inactive tumor suppressor genes. In some embodiments the retrovirus is not genotoxic. In some embodiments the lentivirus integrates into introns.

In some embodiments, the retroviral nucleic acid integrates into the genome of a target cell with a particular copy number. The average copy number may be determined from single cells, a population of cells, or individual cell colonies. Exemplary methods for determining copy number include polymerase chain reaction (PCR) and flow cytometry.

In some embodiments DNA encoding the exogenous agent is integrated into the genome. In some embodiments DNA encoding the exogenous agent is maintained episomally. In some embodiments the ratio of integrated to episomal DNA encoding the exogenous agent is at least 0.01, 0.1, 0.5, 1.0, 2, 5, 10, 100.

In some embodiments DNA encoding the exogenous agent is linear. In some embodiments DNA encoding the exogenous agent is circular. In some embodiments the ratio of linear to circular copies of DNA encoding the exogenous agent is at least 0.01, 0.1, 0.5, 1.0, 2, 5, 10, 100.

In embodiments the DNA encoding the exogenous agent is circular with 1 LTR. In some embodiments the DNA encoding the exogenous agent is circular with 2 LTRs. In some embodiments the ratio of circular, 1 LTR-comprising DNA encoding the exogenous agent to circular, 2 LTR-comprising DNA encoding the exogenous agent is at least 0.1, 0.5, 1.0, 2, 5, 10, 20, 50, 100.

### Maintenance of an Episomal Virus

In retroviruses deficient in integration, circular cDNA off-products of the retrotranscription (e.g., 1-LTR and 2-LTR) can accumulate in the cell nucleus without integrating into the host genome (see Yanez-Munoz R J et ah, Nat. Med. 2006, 12: 348-353). Like other exogenous DNA those intermediates can then integrate in the cellular DNA at equal frequencies (e.g., 10³ to 10⁵/cell).

In some embodiments, episomal retroviral nucleic acid does not replicate. Episomal virus DNA can be modified to be maintained in replicating cells through the inclusion of eukaryotic origin of replication and a scaffold/matrix attachment region (S/MAR) for association with the nuclear matrix.

Thus, in some embodiments, a retroviral nucleic acid described herein comprises a eukaryotic origin of replication or a variant thereof. Examples of eukaryotic origins of replication of interest are the origin of replication of the b-globin gene as have been described by Aladjem et al (Science, 1995, 270: 815-819), a consensus sequence from autonomously replicating sequences associated with alpha-satellite sequences isolated previously from monkey CV-1 cells and human skin fibroblasts as has been described by Price et al Journal of Biological Chemistry, 2003, 278 (22): 19649-59, the origin of replication of the human c-myc promoter region has have been described by McWinney and Leffak (McWinney C. and Leffak M., Nucleic Acid Research 1990, 18(5): 1233-42). In embodiments, the variant substantially maintains the ability to initiate the replication in eukaryotes. The ability of a particular sequence of initiating replication can be determined by any suitable method, for example, the autonomous replication assay based on bromodeoxyuridine incorporation and density shift (Araujo F. D. et ah, supra; Frappier L. et ah, supra).

In some embodiments, the retroviral nucleic acid comprises a scaffold/matrix attachment region (S/MAR) or variant thereof, e.g., a non-consensus-like AT-rich DNA element several hundred base pairs in length, which organizes the nuclear DNA of the eukaryotic genome into chromatin domains, by periodic attachment to the protein scaffold or matrix of the cell nucleus. They are typically found in non-coding regions such as flanking regions, chromatin border regions, and introns. Examples of S/MAR regions are 1.8 kbp S/MAR of the human IFN-g gene (hIFN-y^{large}) as described by Bode et al (Bode J. et ah, Science, 1992, 255: 195-7), the 0.7 Kbp minimal region of the S/MAR of the human IFN-g gene (hIFN—as has have been described by Ramezani (Ramezani A. et ah, Blood 2003, 101: 4717-24), the 0.2 Kbp minimal region of the S/MAR of the human dehydrofolate reductase gene (hDHFR) as has been described by Mesner F. D. et ah, Proc Natl Acad Sci USA, 2003, 100: 3281-86). In embodiments, the functionally equivalent variant of the S/MAR is a sequence selected based on the set six rules that together or alone have been suggested to contribute to S/MAR function (Kramer et al (1996) Genomics 33, 305; Singh et al (1997) Nucl. Acids Res 25, 1419). These rules have been merged into the MAR-Wiz computer program freely available at genomecluster.secs.oakland.edu/MAR-Wiz. In embodiments, the variant substantially maintains the same functions of the S/MAR from which it derives, in particular, the ability to specifically bind to the nuclear the matrix. The skilled person can determine if a particular variant is able to specifically bind to the nuclear matrix, for example by the in vitro or in vivo MAR assays described by Mesner et al. (Mesner F. D. et al, supra). In some embodiments, a specific sequence is a variant of a S/MAR if the particular variant shows propensity for DNA strand separation. This property can be determined using a specific program based on methods from equilibrium statistical mechanics. The stress-induced duplex destabilization (SIDD) analysis technique “[ . . . ] calculates the extent to which the imposed level of superhelical stress decreases the free energy needed to open the duplex at each position along a DNA sequence. The results are displayed as an SIDD profile, in which sites of strong destabilization appear as deep minima [ . . . ]” as defined in Bode et al (2005) J. Mol. Biol. 358,597. The SIDD algorithm and the mathematical basis (Bi and Benham (2004) Bioinformatics 20, 1477) and the analysis of the SIDD profile can be performed using the freely available internet resource at WebSIDD (www.genomecenter.ucdavis.edu/benham). Accordingly, in some embodiments, the polynucleotide is considered a variant of the S/MAR sequence if it shows a similar SIDD profile as the S/MAR.

It is understood that in referring to nucleotide distances between elements in nucleotides, unless specified otherwise, distance refers to the number of nucleotides (of a single strand) or base pairs (in a double strand) that are between the elements but not part of the elements. As an example, if a first element occupies nucleotides 1-100, and a second element occupies nucleotides 102-200 of the same nucleic acid, the distance between the first element and the second element is 1 nucleotide.

Gene Writer Systems with Insulators

A Gene Writer system as described herein may include a template nucleic acid molecule comprising an insulator, a DNA recognition sequence that is specifically bound by a recombinase polypeptide (e.g., a tyrosine recombinase polypeptide or a serine recombinase (e.g., a serine integrase) polypeptide), and a heterologous object sequence. In some embodiments, the insulator is a DNA sequence that can form loop structures via recruitment of insulator proteins, which in turn cause two insulator sequences bound by the insulator proteins to be brought into close proximity with each other. In some instances, the nucleic acid sequence between a first insulator and a second insulator is insulated from one or more of:
a) heterochromatin formation;
b) epigenetic regulation (e.g., from both of epigenetic regulation and transcriptional regulation);
c) transcriptional regulation;
d) histone deacetylation (e.g., from both of histone deacetylation and histone methylation);
e) histone methylation;
f) histone deacetylation; and
g) DNA methylation, e.g., promoter DNA methylation.

In some instances, such insulators can act as barriers to heterochromatin entry into a region of a DNA molecule (e.g., a chromosome). For example, a pair of insulators flanking a region within a DNA molecule may reduce heterochromatin formation and/or presence within the sequence between the insulators, e.g., by at least about 25%, 50%, 75%, 80%, 85%, 90%, 95%, 96%, 97%, 98%, 99%, or 100%. In some instances, the insulators (e.g., by reducing or blocking heterochromatin formation) maintain or increase transcriptional activity of the heterologous object sequence positioned between the insulators. In some instances, transcriptional activity of the heterologous object sequence is maintained at approximately the same level (e.g., within about 75%-80%, 80%-85%, 85%-90%, 90%-95%, 95%-96%, 96%-97%, 97%-98%, 98%-99%, 99%-100%, 100%-110%, or 110%-125% of the level of transcription immediately after integration) over a period of time (e.g., a period of at least 1, 2, 3, 4, 5, 6, 7, 8, 9, 10, 20, 30, 40, 50, or 60 minutes, or a period of at least 1, 2, 3, 4, 5, 6, 7, 8, 9, 10, 11, 12, 14, 16, 18, 20, 22, or 24 hours, or a period of at least 1, 2, 3, 4, 5, 6, 7, 8, 9, 10, 20, 30, 40, 50, 60, 70, 80, 90, or 100 days, or more). In some instances, insulators can have enhancer blocking activity (i.e., reducing or eliminating the activity of an enhancer positioned between two insulator sequences). In some instances, transcriptional activity of a heterologous object sequence flanked by insulators is maintained at a level at least 1%, 2%, 3%, 4%, 5%, 10%, 15%, 20%, 25%, 30%, 40%, 50%, 60%, 70%, 80%, 90%, or 100% higher than the transcriptional activity in an otherwise similar heterologous object sequence not flanked by the insulators, at least 10, 20, 30, 40, 50, or 60 minutes, at least 1, 2, 3, 4, 5, 6, 7, 8, 9, 10, 11, 12, 14, 16, 18, 20, 22, or 24 hours, or at least 1, 2, 3, 4, 5, 6, 7, 8, 9, 10, 20, 30, 40, 50, 60, 70, 80, 90, or 100 days after integration. In some instances, enhancer-blocking insulators can reduce the transcription of a gene regulated by the enhancer by at least about 25%, 50%, 75%, 80%, 85%, 90%, 95%, 96%, 97%, 98%, 99%, or 100%.

In some embodiments, cells treated with a system or method described herein show a decrease in the loss of frequency of expression of the heterologous object sequence at day 28 and/or day 60 after the treatment, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than cells treated with an otherwise similar template nucleic acid lacking the insulators. In some embodiments, cells treated with template nucleic acid comprising the described insulator configuration show a higher frequency of expression and/or a higher level of expression of the heterologous object sequence at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold higher than cells treated with an otherwise similar template nucleic acid lacking the insulators. In some embodiments, cells treated with template nucleic acid comprising the described insulator configuration demonstrate a smaller increase in frequency of expression and/or level of expression of the heterologous object sequence after further treatment with TSA or 5-aza relative to no treatment with TSA or 5-aza, e.g., at least at least 1.1, 1.2, 1.3, 1.4, 1.5, 1.6, 1.7, 1.8, 1.9, 2.0, 2.5, 3, 4, 5, 6, 7, 8, 9, or at least 10-fold smaller increase than cells treated with an otherwise similar template nucleic acid lacking the insulators. In some embodiments, cells treated with template nucleic acid comprising the described insulator configuration demonstrate an increase in frequency of expression and/or level of expression of the heterologous object sequence after further treatment with TSA or 5-aza of less than 10, 9, 8, 7, 6, 5, 4, 3, 2.5, 2.0, 1.9, 1.8, 1.7, 1.6, 1.5, 1.4, 1.3, 1.2, or less than 1.1-fold increase as compared to no treatment with TSA or 5-aza.

In some embodiments, treatment of cells with a system or method described herein results in the formation of fewer IL-3 independent colonies, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold fewer colonies as compared to a an otherwise similar template nucleic acid lacking insulators. In some embodiments, the fraction of mice developing tumors when implanted with cells treated with template nucleic acid comprising an insulator configuration as described herein is lower, e.g., at least 20%, 40%, 60%, 80%, or 100% lower, than mice implanted with cells treated with an otherwise similar template nucleic acid lacking insulators. In some embodiments, the median latency of tumors derived from cells treated with a template nucleic acid comprising an insulator configuration as described herein is longer, e.g., at least 1.1, 1.2, 1.3, 1.4, 1.5, 1.6, 1.7, 1.8, 1.9, or at least 2-fold longer than those derived from cells treated with an otherwise similar template nucleic acid lacking insulators. In some embodiments, the 18-week survival rate of mice implanted with cells treated with template nucleic acid comprising an insulator configuration as described herein is higher, e.g., at least 1.1, 1.2, 1.3, 1.4, 1.5, 1.6, 1.7, 1.8, 1.9, or at least 2-fold higher than that of mice implanted with cells treated with an otherwise similar template nucleic acid lacking insulators.

In some embodiments, treatment of cells with a system or method described herein results in a change in expression that is lower, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than the change in expression after integration using an otherwise similar template nucleic acid lacking insulators for at least one gene local to the site of integration. In some embodiments, integration using a template nucleic acid comprising an insulator configuration as described herein results in a less than 10, 9, 8, 7, 6, 5, 4, 3, 2.5, 2.0, 1.9, 1.8, 1.7, 1.6, 1.5, 1.4, 1.3, 1.2, or a less than 1.1-fold change in gene expression for at least one gene local to the site of integration compared to otherwise similar untreated cells. In some embodiments, integration using a template plasmid comprising an insulator configuration as described herein results in a less than 10, 9, 8, 7, 6, 5, 4, 3, 2.5, 2.0, 1.9, 1.8, 1.7, 1.6, 1.5, 1.4, 1.3, 1.2, or a less than 1.1-fold change in expression for at least 90%, e.g., at least 90%, 95%, 96%, 97%, 97%, 99%, 99.5% or at least 99.9% of global transcripts, compared to otherwise similar untreated cells.

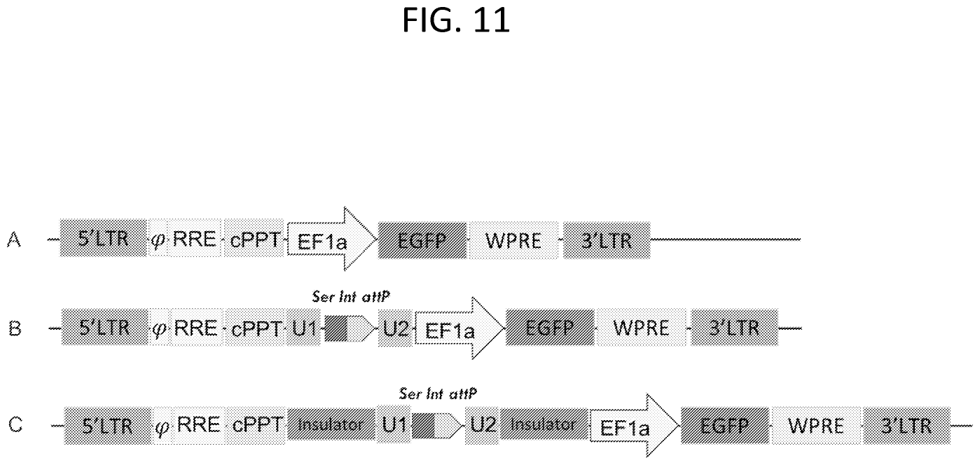

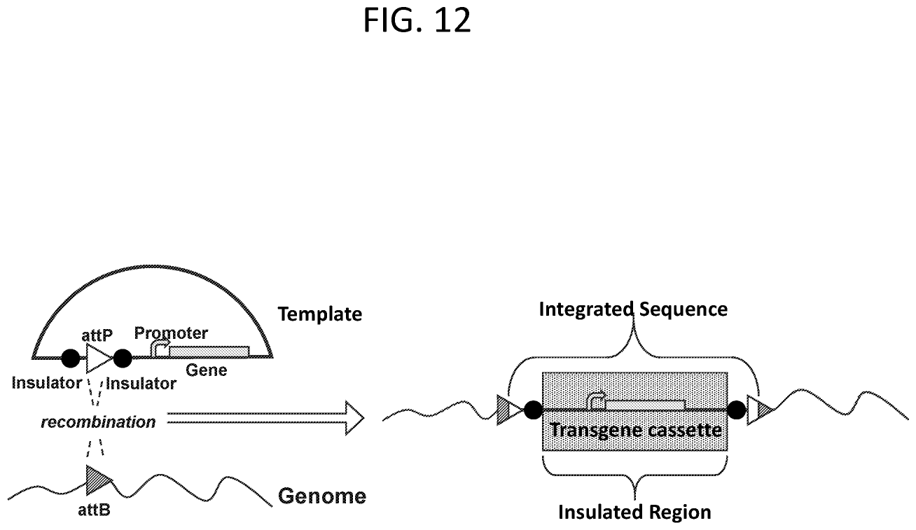

In some instances, a template nucleic acid molecule as described herein comprises a first insulator and a second insulator, with the DNA recognition sequence positioned between the first and second insulator (e.g., as shown in FIGS. 11 and 12). In some instances, the heterologous object sequence is positioned outside of the region between the first and second insulator that comprises the DNA recognition sequence (e.g., as shown in FIGS. 11 and 12). The template nucleic acid molecule may, in some instances, be recombined with a target DNA (e.g., a genomic DNA, e.g., a chromosome or a mitochondrial genome) by a recombinase polypeptide (e.g., a tyrosine recombinase polypeptide or a serine recombinase (e.g., serine integrase) polypeptide), e.g., via recombination of the DNA recognition sequence with a cognate DNA recognition sequence comprised by the target DNA. In some instances, recombination results in integration of the heterologous object sequence into the target DNA, with the first and second insulators flanking the resultant integrated heterologous object sequence.

In some embodiments, the distance between the first insulator and the DNA recognition sequence is less than 2500, 2000, 1500, 1000, 750, 500, 400, 300, 200, 150, 100, 90, 80, 70, 60, 50, 40, 30, 20, 15, 10, 9, 8, 7, 6, 5, 4, 3, 2, or 1 nucleotides. In some embodiments, the distance between the DNA recognition sequence and the second insulator is less than 2500, 2000, 1500, 1000, 750, 500, 400, 300, 200, 150, 100, 90, 80, 70, 60, 50, 40, 30, 20, 15, 10, 9, 8, 7, 6, 5, 4, 3, 2, or 1 nucleotides. In some embodiments, the distance between the first insulator and the second insulator is less than 1000, 900, 800, 700, 600, 500, 400, 300, 200, 150, 100, 90, 80, 70, 60, or 50 nucleotides.

It is understood that in referring to nucleotide distances between elements in nucleotides, unless specified otherwise, distance refers to the number of nucleotides (of a single strand) or base pairs (in a double strand) that are between the elements but not part of the elements. As an example, if a first element occupies nucleotides 1-100, and a second element occupies nucleotides 102-200 of the same nucleic acid, the distance between the first element and the second element is 1 nucleotide.

In some embodiments, the insulator is a chicken P-globin 5′HS4 (cHS4) element, a Scaffold or Matrix Attachment Region (S/MAR) (e.g., MAR X_S29), a Stabilising Anti Repressor (STAR) element (e.g., STAR40), a D4Z4 insulator, A Ubiquitous Chromatin Opening Element (UCOE element) (e.g., aHNRPA2B1-CBX3 locus (A2UCOE), 3′UCOE, or SRF-UCOE), or a functional fragment or variant of any of the foregoing.

In some embodiments, the insulator comprises one or more (e.g., 2, 3, or 4) CAAT-box binding transcription factor binding site (CTF binding site), e.g., as described in Molecular therapy vol. 22 no. 4, 774-785 April 2014, incorporated herein by reference. In some embodiments, the insulator comprises one or more CCCTC-binding factor (also known as CTCF) binding site, e.g., as described in doi:10.1038/nbt.3062, incorporated herein by reference.

In some embodiments, the insulator protein that specifically bounds to one or more insulators (e.g., in a system as described herein) is selected from CTCF (CCCTC-binding factor), CTF (CAAT-binding transcription factor 1), USF1 (Upstream Stimulatory Factor 1), USF2 (Upstream Stimulatory Factor 2), PARP-1 (Poly(ADP-ribose) Polymerase-1), and VEZF1 (Vascular Endothelial Zinc Finger 1), or a polypeptide having at least 75%, 80%, 85%, 90%, 95%, 96%, 97%, 98%, or 99% sequence identity thereto.

### Production of Compositions and Systems

As will be appreciated by one of skill, methods of designing and constructing nucleic acid constructs and proteins or polypeptides (such as the systems, constructs and polypeptides described herein) are routine in the art. Generally, recombinant methods may be used. See, in general, Smales & James (Eds.), Therapeutic Proteins: Methods and Protocols (Methods in Molecular Biology), Humana Press (2005); and Crommelin, Sindelar & Meibohm (Eds.), Pharmaceutical Biotechnology: Fundamentals and Applications, Springer (2013). Methods of designing, preparing, evaluating, purifying and manipulating nucleic acid compositions are described in Green and Sambrook (Eds.), Molecular Cloning: A Laboratory Manual (Fourth Edition), Cold Spring Harbor Laboratory Press (2012).

Exemplary methods for producing a therapeutic pharmaceutical protein or polypeptide described herein involve expression in mammalian cells, although recombinant proteins can also be produced using insect cells, yeast, bacteria, or other cells under control of appropriate promoters. Mammalian expression vectors may comprise non-transcribed elements such as an origin of replication, a suitable promoter, and other 5′ or 3′ flanking non-transcribed sequences, and 5′ or 3′ non-translated sequences such as necessary ribosome binding sites, a polyadenylation site, splice donor and acceptor sites, and termination sequences. DNA sequences derived from the SV40 viral genome, for example, SV40 origin, early promoter, splice, and polyadenylation sites may be used to provide other genetic elements required for expression of a heterologous DNA sequence. Appropriate cloning and expression vectors for use with bacterial, fungal, yeast, and mammalian cellular hosts are described in Green & Sambrook, Molecular Cloning: A Laboratory Manual (Fourth Edition), Cold Spring Harbor Laboratory Press (2012).

Various mammalian cell culture systems can be employed to express and manufacture recombinant protein. Examples of mammalian expression systems include CHO, COS, HEK293, HeLA, and BHK cell lines. Processes of host cell culture for production of protein therapeutics are described in Zhou and Kantardjieff (Eds.), Mammalian Cell Cultures for Biologics Manufacturing (Advances in Biochemical Engineering Biotechnology), Springer (2014). Compositions described herein may include a vector, such as a viral vector, e.g., a lentiviral vector, encoding a recombinant protein. In some embodiments, a vector, e.g., a viral vector, may comprise a nucleic acid encoding a recombinant protein.

Purification of protein therapeutics is described in Franks, Protein Biotechnology: Isolation, Characterization, and Stabilization, Humana Press (2013); and in Cutler, Protein Purification Protocols (Methods in Molecular Biology), Humana Press (2010).

The disclosure is directed, in part, to comparisons of nucleic acid and amino acid sequences with reference sequences or one another to determine % identity or a number of mismatches between said sequences. A person of skill in the art will understand that a number of methods and/or tools are available to make such determinations, including NCBI's BLAST and pairwise alignment tools that perform global sequence alignment of two input sequences (e.g., using the Needleman-Wunsch alignment algorithm) such as the European Bioinformatics Institute (EBI) and European Molecular Biology Laboratory (EMBL) EMBOSS Needle tool.

RNAs (e.g., a gRNA or an mRNA, e.g., an mRNA encoding a GeneWriter) may also be produced as described herein. In some embodiments, RNA segments may be produced by chemical synthesis. In some embodiments, RNA segments may be produced by in vitro transcription of a nucleic acid template, e.g., by providing an RNA polymerase to act on a cognate promoter of a DNA template to produce an RNA transcript. In some embodiments, in vitro transcription is performed using, e.g., a T7, T3, or SP6 RNA polymerase, or a derivative thereof, acting on a DNA, e.g., dsDNA, ssDNA, linear DNA, plasmid DNA, linear DNA amplicon, linearized plasmid DNA, e.g., encoding the RNA segment, e.g., under transcriptional control of a cognate promoter, e.g., a T7, T3, or SP6 promoter. In some embodiments, a combination of chemical synthesis and in vitro transcription is used to generate the RNA segments for assembly. In embodiments, the gRNA is produced by chemical synthesis and the heterologous object sequence segment is produced by in vitro transcription. Without wishing to be bound by theory, in vitro transcription may be better suited for the production of longer RNA molecules. In some embodiments, reaction temperature for in vitro transcription may be lowered, e.g., be less than 37° C. (e.g., between 0-10° C., 10-20° C., or 20-30° C.), to result in a higher proportion of full-length transcripts (see Krieg Nucleic Acids Res 18:6463 (1990), which is herein incorporated by reference in its entirety). In some embodiments, a protocol for improved synthesis of long transcripts is employed to synthesize a long RNA, e.g., an RNA greater than 5 kb, such as the use of e.g., T7 RiboMAX Express, which can generate 27 kb transcripts in vitro (Thiel et al. J Gen Virol 82(6):1273-1281 (2001)). In some embodiments, modifications to RNA molecules as described herein may be incorporated during synthesis of RNA segments (e.g., through the inclusion of modified nucleotides or alternative binding chemistries), following synthesis of RNA segments through chemical or enzymatic processes, following assembly of one or more RNA segments, or a combination thereof.

In some embodiments, an mRNA of the system (e.g., an mRNA encoding a Gene Writer polypeptide) is synthesized in vitro using T7 polymerase-mediated DNA-dependent RNA transcription from a linearized DNA template, where UTP is optionally substituted with 1-methylpseudoUTP. In some embodiments, the transcript incorporates 5′ and 3′ UTRs, e.g., GGGAAAUAAGAGAGAAAAGAAGAGUAAGAAGAAAUAUAAGAGCCACC and UGAUAAUAGGCUGGAGCCUCGGUGGCCAUGCUUCUUGCCCCUUGGGCCUCCCCCC AGCCCCUCCUCCCCUUCCUGCACCCGUACCCCCGUGGUCUUUGAAUAAAGUCUGA, or functional fragments or variants thereof, and optionally includes a poly-A tail, which can be encoded in the DNA template or added enzymatically following transcription. In some embodiments, a donor methyl group, e.g., S-adenosylmethionine, is added to a methylated capped RNA with cap 0 structure to yield a cap 1 structure that increases mRNA translation efficiency (Richner et al. Cell 168(6): P1114-1125 (2017)).

In some embodiments, the transcript from a T7 promoter starts with a GGG motif. In some embodiments, a transcript from a T7 promoter does not start with a GGG motif. It has been shown that a GGG motif at the transcriptional start, despite providing superior yield, may lead to T7 RNAP synthesizing a ladder of poly(G) products as a result of slippage of the transcript on the three C residues in the template strand from +1 to +3 (Imburgio et al. Biochemistry 39(34):10419-10430 (2000). For tuning transcription levels and altering the transcription start site nucleotides to fit alternative 5′ UTRs, the teachings of Davidson et al. Pac Symp Biocomput 433-443 (2010) describe T7 promoter variants, and the methods of discovery thereof, that fulfill both of these traits.

In some embodiments, RNA segments may be connected to each other by covalent coupling. In some embodiments, an RNA ligase, e.g., T4 RNA ligase, may be used to connect two or more RNA segments to each other. When a reagent such as an RNA ligase is used, a 5′ terminus is typically linked to a 3′ terminus. In some embodiments, if two segments are connected, then there are two possible linear constructs that can be formed (i.e., (1) 5′-Segment 1-Segment 2-3′ and (2) 5′-Segment 2-Segment 1-3′). In some embodiments, intramolecular circularization can also occur. Both of these issues can be addressed, for example, by blocking one 5′ terminus or one 3′ terminus so that RNA ligase cannot ligate the terminus to another terminus. In embodiments, if a construct of 5′-Segment 1-Segment 2-3′ is desired, then placing a blocking group on either the 5′ end of Segment 1 or the 3′ end of Segment 2 may result in the formation of only the correct linear ligation product and/or prevent intramolecular circularization. Compositions and methods for the covalent connection of two nucleic acid (e.g., RNA) segments are disclosed, for example, in US20160102322A1 (incorporated herein by reference in its entirety), along with methods including the use of an RNA ligase to directionally ligate two single-stranded RNA segments to each other.

One example of an end blocker that may be used in conjunction with, for example, T4 RNA ligase, is a dideoxy terminator. T4 RNA ligase typically catalyzes the ATP-dependent ligation of phosphodiester bonds between 5′-phosphate and 3′-hydroxyl termini. In some embodiments, when T4 RNA ligase is used, suitable termini must be present on the termini being ligated. One means for blocking T4 RNA ligase on a terminus comprises failing to have the correct terminus format. Generally, termini of RNA segments with a 5-hydroxyl or a 3′-phosphate will not act as substrates for T4 RNA ligase.

Additional exemplary methods that may be used to connect RNA segments is by click chemistry (e.g., as described in U.S. Pat. Nos. 7,375,234 and 7,070,941, and US Patent Publication No. 2013/0046084, the entire disclosures of which are incorporated herein by reference). For example, one exemplary click chemistry reaction is between an alkyne group and an azide group (see FIG. 11 of US20160102322A1, which is incorporated herein by reference in its entirety). Any click reaction may potentially be used to link RNA segments (e.g., Cu-azide-alkyne, strain-promoted-azide-alkyne, staudinger ligation, tetrazine ligation, photo-induced tetrazole-alkene, thiol-ene, NHS esters, epoxides, isocyanates, and aldehyde-aminooxy). In some embodiments, ligation of RNA molecules using a click chemistry reaction is advantageous because click chemistry reactions are fast, modular, efficient, often do not produce toxic waste products, can be done with water as a solvent, and/or can be set up to be stereospecific.

In some embodiments, RNA segments may be connected using an Azide-Alkyne Huisgen Cycloaddition. reaction, which is typically a 1,3-dipolar cycloaddition between an azide and a terminal or internal alkyne to give a 1,2,3-triazole for the ligation of RNA segments. Without wishing to be bound by theory, one advantage of this ligation method may be that this reaction can initiated by the addition of required Cu(I) ions. Other exemplary mechanisms by which RNA segments may be connected include, without limitation, the use of halogens (F—, Br—, I—)/alkynes addition reactions, carbonyls/sulfhydryls/maleimide, and carboxyl/amine linkages. For example, one RNA molecule may be modified with thiol at 3′ (using disulfide amidite and universal support or disulfide modified support), and the other RNA molecule may be modified with acrydite at 5′ (using acrylic phosphoramidite), then the two RNA molecules can be connected by a Michael addition reaction. This strategy can also be applied to connecting multiple RNA molecules stepwise. Also provided are methods for linking more than two (e.g., three, four, five, six, etc.) RNA molecules to each other. Without wishing to be bound by theory, this may be useful when a desired RNA molecule is longer than about 40 nucleotides, e.g., such that chemical synthesis efficiency degrades, e.g., as noted in US20160102322A1 (incorporated herein by reference in its entirety).

By way of illustration, a tracrRNA is typically around 80 nucleotides in length. Such RNA molecules may be produced, for example, by processes such as in vitro transcription or chemical synthesis. In some embodiments, when chemical synthesis is used to produce such RNA molecules, they may be produced as a single synthesis product or by linking two or more synthesized RNA segments to each other. In embodiments, when three or more RNA segments are connected to each other, different methods may be used to link the individual segments together. Also, the RNA segments may be connected to each other in one pot (e.g., a container, vessel, well, tube, plate, or other receptacle), all at the same time, or in one pot at different times or in different pots at different times. In a non-limiting example, to assemble RNA Segments 1, 2 and 3 in numerical order, RNA Segments 1 and 2 may first be connected, 5′ to 3′, to each other. The reaction product may then be purified for reaction mixture components (e.g., by chromatography), then placed in a second pot, for connection of the 3′ terminus with the 5′ terminus of RNA Segment 3. The final reaction product may then be connected to the 5′ terminus of RNA Segment 3.

In another non-limiting example, RNA Segment 1 (about 30 nucleotides) is the target locus recognition sequence of a crRNA and a portion of Hairpin Region 1. RNA Segment 2 (about 35 nucleotides) contains the remainder of Hairpin Region 1 and some of the linear tracrRNA between Hairpin Region 1 and Hairpin Region 2. RNA Segment 3 (about 35 nucleotides) contains the remainder of the linear tracrRNA between Hairpin Region 1 and Hairpin Region 2 and all of Hairpin Region 2. In this example, RNA Segments 2 and 3 are linked, 5′ to 3′, using click chemistry. Further, the 5′ and 3′ end termini of the reaction product are both phosphorylated. The reaction product is then contacted with RNA Segment 1, having a 3′ terminal hydroxyl group, and T4 RNA ligase to produce a guide RNA molecule.

A number of additional linking chemistries may be used to connect RNA segments according to method of the invention. Some of these chemistries are set out in Table 6 of US20160102322A1, which is incorporated herein by reference in its entirety.

### Vectors

The disclosure provides, in part, a nucleic acid, e.g., vector, encoding a Gene Writer polypeptide described herein, a template nucleic acid described herein, or both. In some embodiments, a vector comprises a selective marker, e.g., an antibiotic resistance marker. In some embodiments, the antibiotic resistance marker is a kanamycin resistance marker. In some embodiments, the antibiotic resistance marker does not confer resistance to beta-lactam antibiotics. In some embodiments, the vector does not comprise an ampicillin resistance marker. In some embodiments, the vector comprises a kanamycin resistance marker and does not comprise an ampicillin resistance marker. In some embodiments, a vector encoding a Gene Writer polypeptide is integrated into a target cell genome (e.g., upon administration to a target cell, tissue, organ, or subject). In some embodiments, a vector encoding a Gene Writer polypeptide is not integrated into a target cell genome (e.g., upon administration to a target cell, tissue, organ, or subject). In some embodiments, a vector comprising a template nucleic acid (e.g., template DNA) is not integrated into a target cell genome (e.g., upon administration to a target cell, tissue, organ, or subject). In some embodiments, if a vector is integrated into a target site in a target cell genome, the selective marker is not integrated into the genome. In some embodiments, if a vector is integrated into a target site in a target cell genome, genes or sequences involved in vector maintenance (e.g., plasmid maintenance genes) are not integrated into the genome. In some embodiments, if a vector is integrated into a target site in a target cell genome, transfer regulating sequences (e.g., inverted terminal repeats, e.g., from an AAV) are not integrated into the genome. In some embodiments, administration of a vector (e.g., encoding a Gene Writer polypeptide described herein, a template nucleic acid described herein, or both) to a target cell, tissue, organ, or subject results in integration of a portion of the vector into one or more target sites in the genome(s) of said target cell, tissue, organ, or subject. In some embodiments, less than 99, 95, 90, 80, 70, 60, 50, 40, 30, 20, 10, 5, 4, 3, 2, or 1% of target sites (e.g., no target sites) comprising integrated material comprise a selective marker (e.g., an antibiotic resistance gene), a transfer regulating sequence (e.g., an inverted terminal repeat, e.g., from an AAV), or both from the vector.

### AAV Vectors

In some embodiments, the vector encoding a Gene Writer polypeptide described herein, a template nucleic acid described herein, or both, is an adeno-associated virus (AAV) vector, e.g., comprising an AAV genome. In some embodiments, the AAV genome comprises two genes that encode four replication proteins and three capsid proteins, respectively. In some embodiments, the genes are flanked on either side by 145-bp inverted terminal repeats (ITRs). In some embodiments, the virion comprises up to three capsid proteins (Vp1, Vp2, and/or Vp3), e.g., produced in a 1:1:10 ratio. In some embodiments, the capsid proteins are produced from the same open reading frame and/or from differential splicing (Vp1) and alternative translational start sites (Vp2 and Vp3, respectively). Generally, Vp3 is the most abundant subunit in the virion and participates in receptor recognition at the cell surface defining the tropism of the virus. In some embodiments, Vp1 comprises a phospholipase domain, e.g., which functions in viral infectivity, in the N-terminus of Vp 1.

In some embodiments, packaging capacity of the viral vectors limits the size of the base editor that can be packaged into the vector. For example, the packaging capacity of the AAVs can be about 4.5 kb (e.g., about 3.0, 3.5, 4.0, 4.5, 5.0, 5.5, or 6.0 kb), e.g., including one or two inverted terminal repeats (ITRs), e.g., 145 base ITRs.

In some embodiments, recombinant AAV (rAAV) comprises cis-acting 145-bp ITRs flanking vector transgene cassettes, e.g., providing up to 4.5 kb for packaging of foreign DNA. Subsequent to infection, rAAV can, in some instances, express a protein described herein and persist without integration into the host genome by existing episomally in circular head-to-tail concatemers. rAAV can be used, for example, in vitro and in vivo. In some embodiments, AAV-mediated gene delivery requires that the length of the coding sequence of the gene is equal or greater in size than the wild-type AAV genome.

AAV delivery of genes that exceed this size and/or the use of large physiological regulatory elements can be accomplished, for example, by dividing the protein(s) to be delivered into two or more fragments. In some embodiments, the N-terminal fragment is fused to a split intein-N. In some embodiments, the C-terminal fragment is fused to a split intein-C. In embodiments, the fragments are packaged into two or more AAV vectors.

In some embodiments, dual AAV vectors are generated by splitting a large transgene expression cassette in two separate halves (5 and 3 ends, or head and tail), e.g., wherein each half of the cassette is packaged in a single AAV vector (of <5 kb). The re-assembly of the full-length transgene expression cassette can, in some embodiments, then be achieved upon co-infection of the same cell by both dual AAV vectors. In some embodiments, co-infection is followed by one or more of: (1) homologous recombination (HR) between 5 and 3 genomes (dual AAV overlapping vectors); (2) ITR-mediated tail-to-head concatemerization of 5 and 3 genomes (dual AAV trans-splicing vectors); and/or (3) a combination of these two mechanisms (dual AAV hybrid vectors). In some embodiments, the use of dual AAV vectors in vivo results in the expression of full-length proteins. In some embodiments, the use of the dual AAV vector platform represents an efficient and viable gene transfer strategy for transgenes of greater than about 4.0, 4.1, 4.2, 4.3, 4.4, 4.5, 4.6, 4.7, 4.8, 4.9, or 5.0 kb in size. In some embodiments, AAV vectors can also be used to transduce cells with target nucleic acids, e.g., in the in vitro production of nucleic acids and peptides. In some embodiments, AAV vectors can be used for in vivo and ex vivo gene therapy procedures (see, e.g., West et al., Virology 160:38-47 (1987); U.S. Pat. No. 4,797,368; WO 93/24641; Kotin, Human Gene Therapy 5:793-801 (1994); Muzyczka, J. Clin. Invest.94:1351 (1994); each of which is incorporated herein by reference in their entirety). The construction of recombinant AAV vectors is described in a number of publications, including U.S. Pat. No. 5,173,414; Tratschin et al., Mol. Cell. Biol.5:3251-3260 (1985); Tratschin, et al., Mol. Cell. Biol.4:2072-2081 (1984); Hermonat & Muzyczka, PNAS 81:6466-6470 (1984); and Samulski et al., J. Virol.63:03822-3828 (1989) (incorporated by reference herein in their entirety).

In some embodiments, a Gene Writer described herein (e.g., with or without one or more guide nucleic acids) can be delivered using AAV, lentivirus, adenovirus or other plasmid or viral vector types, in particular, using formulations and doses from, for example, U.S. Pat. No. 8,454,972 (formulations, doses for adenovirus), U.S. Pat. No. 8,404,658 (formulations, doses for AAV) and U.S. Pat. No. 5,846,946 (formulations, doses for DNA plasmids) and from clinical trials and publications regarding the clinical trials involving lentivirus, AAV and adenovirus. For example, for AAV, the route of administration, formulation and dose can be as described in U.S. Pat. No. 8,454,972 and as in clinical trials involving AAV. For Adenovirus, the route of administration, formulation and dose can be as described in U.S. Pat. No. 8,404,658 and as in clinical trials involving adenovirus. For plasmid delivery, the route of administration, formulation and dose can be as described in U.S. Pat. No. 5,846,946 and as in clinical studies involving plasmids. Doses can be based on or extrapolated to an average 70 kg individual (e.g. a male adult human), and can be adjusted for patients, subjects, mammals of different weight and species. Frequency of administration is within the ambit of the medical or veterinary practitioner (e.g., physician, veterinarian), depending on usual factors including the age, sex, general health, other conditions of the patient or subject and the particular condition or symptoms being addressed. In some embodiments, the viral vectors can be injected into the tissue of interest. For cell-type specific Gene Writing, the expression of the Gene Writer and optional guide nucleic acid can, in some embodiments, be driven by a cell-type specific promoter.

In some embodiments, AAV allows for low toxicity, for example, due to the purification method not requiring ultracentrifugation of cell particles that can activate the immune response. In some embodiments, AAV allows low probability of causing insertional mutagenesis, for example, because it does not substantially integrate into the host genome.

In some embodiments, AAV has a packaging limit of about 4.4, 4.5, 4.6, 4.7, or 4.75 kb. In some embodiments, a Gene Writer, promoter, and transcription terminator can fit into a single viral vector. SpCas9 (4.1 kb) may, in some instances, be difficult to package into AAV. Therefore, in some embodiments, a Gene Writer is used that is shorter in length than other Gene Writers or base editors. In some embodiments, the Gene Writers are less than about 4.5 kb, 4.4 kb, 4.3 kb, 4.2 kb, 4.1 kb, 4 kb, 3.9 kb, 3.8 kb, 3.7 kb, 3.6 kb, 3.5 kb, 3.4 kb, 3.3 kb, 3.2 kb, 3.1 kb, 3 kb, 2.9 kb, 2.8 kb, 2.7 kb, 2.6 kb, 2.5 kb, 2 kb, or 1.5 kb.

An AAV can be AAV1, AAV2, AAV5 or any combination thereof. In some embodiments, the type of AAV is selected with respect to the cells to be targeted; e.g., AAV serotypes 1, 2, 5 or a hybrid capsid AAV1, AAV2, AAV5 or any combination thereof can be selected for targeting brain or neuronal cells; or AAV4 can be selected for targeting cardiac tissue. In some embodiments, AAV8 is selected for delivery to the liver. Exemplary AAV serotypes as to these cells are described, for example, in Grimm, D. et al, J. Virol.82: 5887-5911 (2008) (incorporated herein by reference in its entirety). In some embodiments, AAV refers all serotypes, subtypes, and naturally-occurring AAV as well as recombinant AAV. AAV may be used to refer to the virus itself or a derivative thereof. In some embodiments, AAV includes AAV1, AAV2, AAV3, AAV3, AAV4, AAV5, AAV6, AAV6.2, AAV7, AAVrh.64R1, AAVhu.37, AAVrh.8, AAVrh.32.33, AAV8, AAV9, AAV-DJ, AAV2/8, AAVrh10, AAVLK3, AV 1O, AAV 11, AAV 12, rhG, and hybrids thereof, avian AAV, bovine AAV, canine AAV, equine AAV, primate AAV, nonprimate AAV, and ovine AAV. The genomic sequences of various serotypes ofAAV, as well as the sequences ofthe native terminal repeats (TRs), Rep proteins, and capsid subunits are known in the art. Such sequences may be found in the literature or in public databases such as Genflank. Additional exemplary AAV serotypes are listed in Table 13.

TABLE 13
Viral delivery modalities

| Target | | |
|---|---|---|
| Tissue | Vehicle | Reference |
| Liver | AAV (AAV5, AAV6, | 1. Wang et al., Mol. Ther. 18, |
| | AAV8¹, AAVrh.8¹, | 118-25 (2010) |
| | AAVhu.37¹, AAV2/8, | 2. Ginn et al., JHEP Reports, |
| | AAV2/rh10², AAV9, | 100065 (2019) |
| | AAV2, NP40³, NP59^{2, 3}, | 3. Paulk et al., Mol. Ther. 26, |
| | AAV3B⁵, AAV-DJ⁴, | 289-303 (2018). |
| | AAV-LK01⁴, AAV-LK02⁴, | 4. L. Lisowski et al., Nature. |
| | AAV-LK03⁴, AAV-LK19⁴ | 506, 382-6 (2014). |
| | Adenovirus (Ad5, | 5. L. Wang et al., Mol. Ther. 23, |
| | HC-AdV⁶) | 1877-87 (2015). |
| | | 6. Hausl Mol Ther (2010) |
| Lung | AAV (AAV4, AAV5, | 1. Duncan et al., Mol Ther |
| | AAV6¹, AAV9, H22²) | Methods Clin Dev (2018) |
| | Adenovirus (Ad5, Ad3, | 2. Cooney et al., Am J Respir |
| | Ad21, Ad14)³ | Cell Mol Biol (2019) |
| | | 3. Li et al., Mol Ther Methods |
| | | Clin Dev (2019) |
| Skin | AAV6¹, AAV-LK19² | 1. Petek et al., Mol. Ther. (2010) |
| | | 2. L. Lisowski et al., Nature. |
| | | 506, 382-6 (2014). |
| HSCs | HDAd5/35⁺⁺ | Wang et al. Blood Adv (2019) |

In some embodiments, a pharmaceutical composition (e.g., comprising an AAV as described herein) has less than 1000 empty capsids, less than 8% empty capsids, less than 7% empty capsids, less than 500 empty capsids, less than 30% empty capsids, or less than 1% empty capsids. In some embodiments, the pharmaceutical composition has less than about 5% empty capsids. In some embodiments, the number of empty capsids is below the limit of detection. In some embodiments, it is advantageous for the pharmaceutical composition to have low amounts of empty capsids, e.g., because empty capsids may generate an adverse response (e.g., immune response, inflammatory response, liver response, and/or cardiac response), e.g., with little or no substantial therapeutic benefit.

In some embodiments, the residual host cell protein (rHCP) in the pharmaceutical composition is less than or equal to 100 ng/ml rHCP per 1×10¹³ vg/ml, e.g., less than or equal to 40 ng/ml rHCP per 1×10¹³ vg/ml or 1-50 ng/ml rHCP per 1×10¹³ vg/ml. In some embodiments, the pharmaceutical composition comprises less than 10 ng rHCP per 1.0×10¹³ vg, or less than 5 ng rHCP per 1.0×10¹³ vg, less than 4 ng rHCP per 1.0×10¹³ vg, or less than 3 ng rHCP per 1.0×10¹³ vg, or any concentration in between. In some embodiments, the residual host cell DNA (hcDNA) in the pharmaceutical composition is less than or equal to 5×10⁶ pg/ml hcDNA per 1×10¹³ vg/ml, less than or equal to 1.2×10⁶ pg/ml hcDNA per 1×10¹³ vg/ml, or 1×10⁵ pg/ml hcDNA per 1×10¹³ vg/ml. In some embodiments, the residual host cell DNA in said pharmaceutical composition is less than 5.0×10⁵ pg per 1×10¹³ vg, less than 2.0×10⁵ pg per 1.0×10¹³ vg, less than 1.1×10⁵ μg per 1.0×10¹³ vg, less than 1.0×10⁵ pg hcDNA per 1.0×10¹³ vg, less than 0.9×10⁵ pg hcDNA per 1.0×10¹³ vg, less than 0.8×10⁵ pg hcDNA per 1.0×10¹³ vg, or any concentration in between.

In some embodiments, the residual plasmid DNA in the pharmaceutical composition is less than or equal to 1.7×10⁵ pg/ml per 1.0×10¹³ vg/ml, or 1×10⁵ pg/ml per 1×1.0×10¹³ vg/ml, or 1.7×10⁶ pg/ml per 1.0×10¹³ vg/ml. In some embodiments, the residual DNA plasmid in the pharmaceutical composition is less than 10.0×10 5 pg by 1.0×10¹³ vg, less than 8.0×10⁵ pg by 1.0×10¹³ vg or less than 6.8×10 5 pg by 1.0×10¹³ vg. In embodiments, the pharmaceutical composition comprises less than 0.5 ng per 1.0×10¹³ vg, less than 0.3 ng per 1.0×10¹³ vg, less than 0.22 ng per 1.0×10¹³ vg or less than 0.2 ng per 1.0×10¹³ vg or any intermediate concentration of bovine serum albumin (BSA). In embodiments, the benzonase in the pharmaceutical composition is less than 0.2 ng by 1.0×10¹³ vg, less than 0.1 ng by 1.0×10¹³ vg, less than 0.09 ng by 1.0×10¹³ vg, less than 0.08 ng by 1.0×10¹³ vg or any intermediate concentration. In embodiments, Poloxamer 188 in the pharmaceutical composition is about 10 to 150 ppm, about 15 to 100 ppm or about 20 to 80 ppm. In embodiments, the cesium in the pharmaceutical composition is less than 50 pg/g (ppm), less than 30 pg/g (ppm) or less than 20 pg/g (ppm) or any intermediate concentration.

In embodiments, the pharmaceutical composition comprises total impurities, e.g., as determined by SDS-PAGE, of less than 10%, less than 8%, less than 7%, less than 6%, less than 5%, less than 4%, less than 3%, less than 2%, or any percentage in between. In embodiments, the total purity, e.g., as determined by SDS-PAGE, is greater than 90%, greater than 92%, greater than 93%, greater than 94%, greater than 95%, greater than 96%, greater than 97%, greater than 98%, or any percentage in between. In embodiments, no single unnamed related impurity, e.g., as measured by SDS-PAGE, is greater than 5%, greater than 4%, greater than 3% or greater than 2%, or any percentage in between. In embodiments, the pharmaceutical composition comprises a percentage of filled capsids relative to total capsids (e.g., peak 1+peak 2 as measured by analytical ultracentrifugation) of greater than 85%, greater than 86%, greater than 87%, greater than 88%, greater than 89%, greater than 90%, greater than 91%, greater than 91.9%, greater than 92%, greater than 93%, or any percentage in between. In embodiments of the pharmaceutical composition, the percentage of filled capsids measured in peak 1 by analytical ultracentrifugation is 20-80%, 25-75%, 30-75%, 35-75%, or 37.4-70.3%. In embodiments of the pharmaceutical composition, the percentage of filled capsids measured in peak 2 by analytical ultracentrifugation is 20-80%, 20-70%, 22-65%, 24-62%, or 24.9-60.1%.

In one embodiment, the pharmaceutical composition comprises a genomic titer of 1.0 to 5.0×10¹³ vg/mL, 1.2 to 3.0×10¹³ vg/mL or 1.7 to 2.3×10¹³ vg/ml. In one embodiment, the pharmaceutical composition exhibits a biological load of less than 5 CFU/mL, less than 4 CFU/mL, less than 3 CFU/mL, less than 2 CFU/mL or less than 1 CFU/mL or any intermediate contraction. In embodiments, the amount of endotoxin according to USP, for example, USP<85> (incorporated by reference in its entirety) is less than 1.0 EU/mL, less than 0.8 EU/mL or less than 0.75 EU/mL. In embodiments, the osmolarity of a pharmaceutical composition according to USP, for example, USP<785> (incorporated by reference in its entirety) is 350 to 450 mOsm/kg, 370 to 440 mOsm/kg or 390 to 430 mOsm/kg. In embodiments, the pharmaceutical composition contains less than 1200 particles that are greater than 25 m per container, less than 1000 particles that are greater than 25 m per container, less than 500 particles that are greater than 25 m per container or any intermediate value. In embodiments, the pharmaceutical composition contains less than 10,000 particles that are greater than 10 m per container, less than 8000 particles that are greater than 10 m per container or less than 600 particles that are greater than 10 μm per container.

In one embodiment, the pharmaceutical composition has a genomic titer of 0.5 to 5.0×10¹³ vg/mL, 1.0 to4.0×10¹³ vg/mL, 1.5 to3.0×10¹³ vg/ml or1.7 to2.3×10¹³ vg/m 1 In one embodiment, the pharmaceutical composition described herein comprises one or more of the following: less than about 0.09 ng benzonase per 1.0×10¹³ vg, less than about 30 pg/g (ppm) of cesium, about 20 to 80 ppm Poloxamer 188, less than about 0.22 ng BSA per 1.0×10¹³ vg, less than about 6.8×10 5 μg of residual DNA plasmid per 1.0×10¹³ vg, less than about 1.1×10⁵ pg of residual hcDNA per 1.0×10¹³ vg, less than about 4 ng of rHCP per 1.0×10¹³ vg, pH 7.7 to 8.3, about 390 to 430 mOsm/kg, less than about 600 particles that are >25 m in size per container, less than about 6000 particles that are >10 m in size per container, about 1.7×10¹³-2.3×10¹³ vg/mL genomic titer, infectious titer of about 3.9×10⁸ to 8.4×10¹⁰ IU per 1.0×10¹³ vg, total protein of about 100-300 pg per 1.0×10¹³ vg, mean survival of >24 days in A7SMA mice with about 7.5×10¹³ vg/kg dose of viral vector, about 70 to 130% relative potency based on an in vitro cell based assay and/or less than about 5% empty capsid. In various embodiments, the pharmaceutical compositions described herein comprise any of the viral particles discussed here, retain a potency of between ±20%, between +15%, between ±10% or within ±5% of a reference standard. In some embodiments, potency is measured using a suitable in vitro cell assay or in vivo animal model.

Additional methods of preparation, characterization, and dosing AAV particles are taught in WO2019094253, which is incorporated herein by reference in its entirety.

Additional rAAV constructs that can be employed consonant with the invention include those described in Wang et al 2019, available at: //doi.org/10.1038/s41573-019-0012-9, including Table 1 thereof, which is incorporated by reference in its entirety.

### Kits, Articles of Manufacture, and Pharmaceutical Compositions

In an aspect the disclosure provides a kit comprising a Gene Writer or a Gene Writing system, e.g., as described herein. In some embodiments, the kit comprises a Gene Writer polypeptide (or a nucleic acid encoding the polypeptide) and a template DNA. In some embodiments, the kit further comprises a reagent for introducing the system into a cell, e.g., transfection reagent, LNP, and the like. In some embodiments, the kit is suitable for any of the methods described herein. In some embodiments, the kit comprises one or more elements, compositions (e.g., pharmaceutical compositions), Gene Writers, and/or Gene Writer systems, or a functional fragment or component thereof, e.g., disposed in an article of manufacture. In some embodiments, the kit comprises instructions for use thereof.

In an aspect, the disclosure provides an article of manufacture, e.g., in which a kit as described herein, or a component thereof, is disposed.

In an aspect, the disclosure provides a pharmaceutical composition comprising a Gene Writer or a Gene Writing system, e.g., as described herein. In some embodiments, the pharmaceutical composition further comprises a pharmaceutically acceptable carrier or excipient. In some embodiments, the pharmaceutical composition comprises a template DNA.

### Chemistry, Manufacturing, and Controls (CMC)

Purification of protein therapeutics is described, for example, in Franks, Protein Biotechnology: Isolation, Characterization, and Stabilization, Humana Press (2013); and in Cutler, Protein Purification Protocols (Methods in Molecular Biology), Humana Press (2010).

In some embodiments, a Gene Writer™ system, polypeptide, and/or template nucleic acid (e.g., template DNA) conforms to certain quality standards. In some embodiments, a Gene Writer™ system, polypeptide, and/or template nucleic acid (e.g., template DNA) produced by a method described herein conforms to certain quality standards. Accordingly, the disclosure is directed, in some aspects, to methods of manufacturing a Gene Writer™ system, polypeptide, and/or template nucleic acid that conforms to certain quality standards, e.g., in which said quality standards are assayed. The disclosure is also directed, in some aspects, to methods of assaying said quality standards in a Gene Writer™ system, polypeptide, and/or template nucleic acid. In some embodiments, quality standards include, but are not limited to, one or more (e.g., 1, 2, 3, 4, 5, 6, 7, 8, 9, 10, 11, or 12) of the following:
(i) the length of the template DNA or the mRNA encoding the GeneWriter polypeptide, e.g., whether the DNA or mRNA has a length that is above a reference length or within a reference length range, e.g., whether at least 80, 85, 90, 95, 96, 97, 98, or 99% of the DNA or mRNA present is greater than 100, 125, 150, 175, or 200 nucleotides long;
(ii) the presence, absence, and/or length of a polyA tail on the mRNA, e.g., whether at least 80, 85, 90, 95, 96, 97, 98, or 99% of the mRNA present contains a polyA tail (e.g., a polyA tail that is at least 5, 10 (SEQ ID NO: 3540), 20 (SEQ ID NO: 3541), 30 (SEQ ID NO: 3542), 50 (SEQ ID NO: 3543), 70 (SEQ ID NO: 3544), 100 (SEQ ID NO: 3545) nucleotides in length);
(iii) the presence, absence, and/or type of a 5′ cap on the mRNA, e.g., whether at least 80, 85, 90, 95, 96, 97, 98, or 99% of the mRNA present contains a 5′ cap, e.g., whether that cap is a 7-methylguanosine cap, e.g., a O-Me-m7G cap;
(iv) the presence, absence, and/or type of one or more modified nucleotides (e.g., selected from pseudouridine, dihydrouridine, inosine, 7-methylguanosine, 1-N-methylpseudouridine (1-Me-Ψ), 5-methoxyuridine (5-MO-U), 5-methylcytidine (5mC), or a locked nucleotide) in the mRNA, e.g., whether at least 80, 85, 90, 95, 96, 97, 98, or 99% of the mRNA present contains one or more modified nucleotides;
(v) the stability of the template DNA or the mRNA (e.g., over time and/or under a pre-selected condition), e.g., whether at least 80, 85, 90, 95, 96, 97, 98, or 99% of the DNA or mRNA remains intact (e.g., greater than 100, 125, 150, 175, or 200 nucleotides long) after a stability test;
(vi) the potency of the template DNA or the mRNA in a system for modifying DNA, e.g., whether at least 1% of target sites are modified after a system comprising the DNA or mRNA is assayed for potency;
(vii) the length of the polypeptide, first polypeptide, or second polypeptide, e.g., whether the polypeptide, first polypeptide, or second polypeptide has a length that is above a reference length or within a reference length range, e.g., whether at least 80, 85, 90, 95, 96, 97, 98, or 99% of the polypeptide, first polypeptide, or second polypeptide present is greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long);
(viii) the presence, absence, and/or type of post-translational modification on the polypeptide, first polypeptide, or second polypeptide, e.g., whether at least 80, 85, 90, 95, 96, 97, 98, or 99% of the polypeptide, first polypeptide, or second polypeptide contains phosphorylation, methylation, acetylation, myristoylation, palmitoylation, isoprenylation, glipyatyon, or lipoylation, or any combination thereof,
(ix) the presence, absence, and/or type of one or more artificial, synthetic, or non-canonical amino acids (e.g., selected from ornithine, P-alanine, GABA, 6-Aminolevulinic acid, PABA, a D-amino acid (e.g., D-alanine or D-glutamate), aminoisobutyric acid, dehydroalanine, cystathionine, lanthionine, Djenkolic acid, Diaminopimelic acid, Homoalanine, Norvaline, Norleucine, Homonorleucine, homoserine, O-methyl-homoserine and O-ethyl-homoserine, ethionine, selenocysteine, selenohomocysteine, selenomethionine, selenoethionine, tellurocysteine, or telluromethionine) in the polypeptide, first polypeptide, or second polypeptide, e.g., whether at least 80, 85, 90, 95, 96, 97, 98, or 99% of the polypeptide, first polypeptide, or second polypeptide present contains one or more artificial, synthetic, or non-canonical amino acids;
(x) the stability of the polypeptide, first polypeptide, or second polypeptide (e.g., over time and/or under a pre-selected condition), e.g., whether at least 80, 85, 90, 95, 96, 97, 98, or 99% of the polypeptide, first polypeptide, or second polypeptide remains intact (e.g., greater than 600, 650, 700, 750, 800, 850, 900, 950, 1000, 1050, 1100, 1150, 1200, 1250, 1300, 1350, 1400, 1450, 1500, 1600, 1700, 1800, 1900, or 2000 amino acids long (and optionally, no larger than 2500, 2000, 1500, 1400, 1300, 1200, 1100, 1000, 900, 800, 700, or 600 amino acids long)) after a stability test;
(xi) the potency of the polypeptide, first polypeptide, or second polypeptide in a system for modifying DNA, e.g., whether at least 1% of target sites are modified after a system comprising the polypeptide, first polypeptide, or second polypeptide is assayed for potency; or
(xii) the presence, absence, and/or level of one or more of a pyrogen, virus, fungus, bacterial pathogen, or host cell protein, e.g., whether the system is free or substantially free of pyrogen, virus, fungus, bacterial pathogen, or host cell protein contamination.

In some embodiments, a system or pharmaceutical composition described herein is endotoxin free.

In some embodiments, the presence, absence, and/or level of one or more of a pyrogen, virus, fungus, bacterial pathogen, and/or host cell protein is determined. In embodiments, whether the system is free or substantially free of pyrogen, virus, fungus, bacterial pathogen, and/or host cell protein contamination is determined.

In some embodiments, a pharmaceutical composition or system as described herein has one or more (e.g., 1, 2, 3, or 4) of the following characteristics:
(a) less than 1% (e.g., less than 0.5%, 0.4%, 0.3%, 0.2%, or 0.1%) DNA template relative to the RNA encoding the polypeptide, e.g., on a molar basis;
(b) less than 1% (e.g., less than 0.5%, 0.4%, 0.3%, 0.2%, or 0.1%) uncapped RNA relative to the RNA encoding the polypeptide, e.g., on a molar basis;
(c) less than 1% (e.g., less than 0.5%, 0.4%, 0.3%, 0.2%, or 0.1%) partial length RNAs relative to the RNA encoding the polypeptide, e.g., on a molar basis;
(d) substantially lacks unreacted cap dinucleotides.

### Exemplary Heterologous Object Sequences

In some embodiments, the systems or methods provided herein comprise a heterologous object sequence, wherein the heterologous object sequence or a reverse complementary sequence thereof, encodes a protein (e.g., an antibody) or peptide. In some embodiments, the therapy is one approved by a regulatory agency such as FDA.

In some embodiments, the protein or peptide is a protein or peptide from the THPdb database (Usmani et al. PLoS One 12(7):e0181748 (2017), herein incorporated by reference in its entirety. In some embodiments, the protein or peptide is a protein or peptide disclosed in Table 14. In some embodiments, the systems or methods disclosed herein, for example, those comprising Gene Writers, may be used to integrate an expression cassette for a protein or peptide from Table 14 into a host cell to enable the expression of the protein or peptide in the host. In some embodiments, the sequences of the protein or peptide in the first column of Table 14 can be found in the patents or applications provided in the third column of Table 14, incorporated by reference in their entireties.

In some embodiments, the protein or peptide is an antibody disclosed in Table 1 of Lu et al. J Biomed Sci 27(1):1 (2020), herein incorporated by reference in its entirety. In some embodiments, the protein or peptide is an antibody disclosed in Table 15. In some embodiments, the systems or methods disclosed herein, for example, those comprising Gene Writers, may be used to integrate an expression cassette for an antibody from Table 15 into a host cell to enable the expression of the antibody in the host. In some embodiments, a system or method described herein is used to express an agent that binds a target of column 2 of Table 15 (e.g., a monoclonal antibody of column 1 of Table 15) in a subject having an indication of column 3 of Table 15.

TABLE 14
Exemplary protein and peptide therapeutics.

| Therapeutic peptide | Category | Patent Number |
|---|---|---|
| Lepirudin | Antithrombins and Fibrinolytic | CA1339104 |
| | Agents | |
| Cetuximab | Antineoplastic Agents | CA1340417 |
| Dor se alpha | Enzymes | CA2184581 |
| Denileukin diftitox | Antineoplastic Agents | |
| Etanercept | Immunosuppressive Agents | CA2476934 |
| Bivalirudin | Antithrombins | US7582727 |
| Leuprolide | Antineoplastic Agents | |
| Peginterferon alpha-2a | Immunosuppressive Agents | CA2203480 |
| Alteplase | Thrombolytic Agents | |
| Interferon alpha-n1 | Antiviral Agents | |
| Darbepoetin alpha | Anti-anemic Agents | CA2165694 |
| Reteplase | Fibrinolytic Agents | CA2107476 |
| Epoetin alpha | Hematinics | CA1339047 |
| Salmon Calcitonin | Bone Density Conservation | US6440392 |
| | Agents | |
| Interferon alpha-n3 | Immunosuppressive Agents | |
| Pegfilgrastim | Immunosuppressive Agents | CA1341537 |
| Sargramostim | Immunosuppressive Agents | CA1341150 |
| Secretin | Diagnostic Agents | |
| Peginterferon alpha-2b | Immunosuppressive Agents | CA1341567 |
| Asparagi se | Antineoplastic Agents | |
| Thyrotropin alpha | Diagnostic Agents | US5840566 |
| Antihemophilic Factor | Coagulants and Thrombotic agents | CA2124690 |
| A kinra | Antirheumatic Agents | CA2141953 |
| Gramicidin D | Anti-Bacterial Agents | |
| Intravenous | Immunologic Factors | |
| Immunoglobulin | | |
| Anistreplase | Fibrinolytic Agents | |
| Insulin Regular | Antidiabetic Agents | |
| Tenecteplase | Fibrinolytic Agents | CA2129660 |
| Menotropins | Fertility Agents | |
| Interferon gamma-1b | Immunosuppressive Agents | US6936695 |
| Interferon alpha-2a, | | CA2172664 |
| Recombi nt | | |
| Coagulation factor VIIa | Coagulants | |
| Oprelvekin | Antineoplastic Agents | |
| Palifermin | Anti-Mucositis Agents | |
| Glucagon recombi nt | Hypoglycemic Agents | |
| Aldesleukin | Antineoplastic Agents | |
| Botulinum Toxin Type B | Antidystonic Agents | |
| Omalizumab | Anti-Allergic Agents | CA2113813 |
| Lutropin alpha | Fertility Agents | US5767251 |
| Insulin Lispro | Hypoglycemic Agents | US5474978 |
| Insulin Glargine | Hypoglycemic Agents | US7476652 |
| Collage se | | |
| Rasburicase | Gout Suppressants | CA2175971 |
| Adalimumab | Antirheumatic Agents | CA2243459 |
| Imiglucerase | Enzyme Replacement Agents | US5549892 |
| Abciximab | Anticoagulants | CA1341357 |
| Alpha-1-protei se inhibitor | Serine Protei se Inhibitors | |
| Pegaspargase | Antineoplastic Agents | |
| Interferon beta-1a | Antineoplastic Agents | CA1341604 |
| Pegademase bovine | Enzyme Replacement Agents | |
| Human Serum Albumin | Serum substitutes | US6723303 |
| Eptifibatide | Platelet Aggregation Inhibitors | US6706681 |
| Serum albumin iodo ted | Diagnostic Agents | |
| Infliximab | Antirheumatic Agents, | CA2106299 |
| | Anti-Inflammatory Agents, | |
| | Non-Steroidal, Dermatologic | |
| | Agents, Gastrointesti l Agents | |
| | and Immunosuppressive Agents | |
| Follitropin beta | Fertility Agents | US7741268 |
| Vasopressin | Antidiuretic Agents | |
| Interferon beta-1b | Adjuvants, Immunologic and | CA1340861 |
| | Immunosuppressive Agents | |
| Interferon alphacon-1 | Antiviral Agents and | CA1341567 |
| | Immunosuppressive Agents | |
| Hyaluronidase | Adjuvants, Anesthesia and | |
| | Permeabilizing Agents | |
| Insulin, porcine | Hypoglycemic Agents | |
| Trastuzumab | Antineoplastic Agents | CA2103059 |
| Rituximab | Antineoplastic Agents, | CA2149329 |
| | Immunologic Factors and | |
| | Antirheumatic Agents | |
| Basiliximab | Immunosuppressive Agents | CA2038279 |
| Muromo b | Immunologic Factors and | |
| | Immunosuppressive Agents | |
| Digoxin Immune Fab | Antidotes | |
| (Ovine) | | |
| Ibritumomab | | CA2149329 |
| Daptomycin | | US6468967 |
| Tositumomab | | |
| Pegvisomant | Hormone Replacement Agents | US5849535 |
| Botulinum Toxin Type A | Neuromuscular Blocking Agents, | CA2280565 |
| | Anti-Wrinkle Agents and | |
| | Antidystonic Agents | |
| Pancrelipase | Gastrointesti l Agents and Enzyme | |
| | Replacement Agents | |
| Streptoki se | Fibrinolytic Agents and | |
| | Thrombolytic Agents | |
| Alemtuzumab | | CA1339198 |
| Alglucerase | Enzyme Replacement Agents | |
| Capromab | Indicators, Reagents and | |
| | Diagnostic Agents | |
| Laronidase | Enzyme Replacement Agents | |
| Urofollitropin | Fertility Agents | US5767067 |
| Efalizumab | Immunosuppressive Agents | |
| Serum albumin | Serum substitutes | US6723303 |
| Choriogo dotropin alpha | Fertility Agents and Go dotropins | US6706681 |
| Antithymocyte globulin | Immunologic Factors and | |
| | Immunosuppressive Agents | |
| Filgrastim | Immunosuppressive Agents, | CA1341537 |
| | Antineutropenic Agents and | |
| | Hematopoietic Agents | |
| Coagulation factor ix | Coagulants and Thrombotic | |
| | Agents | |
| Becaplermin | Angiogenesis Inducing Agents | CA1340846 |
| Agalsidase beta | Enzyme Replacement Agents | CA2265464 |
| Interferon alpha-2b | Immunosuppressive Agents | CA1341567 |
| Oxytocin | Oxytocics, Anti-tocolytic Agents | |
| | and Labor Induction Agents | |
| Enfuvirtide | HIV Fusion Inhibitors | US6475491 |
| Palivizumab | Antiviral Agents | CA2197684 |
| Daclizumab | Immunosuppressive Agents | |
| Bevacizumab | Angiogenesis Inhibitors | CA2286330 |
| Arcitumomab | Diagnostic Agents | US8420081 |
| Arcitumomab | Diagnostic Agents | US7790142 |
| Eculizumab | | CA2189015 |
| Panitumumab | | |
| Ranibizumab | Ophthalmics | CA2286330 |
| Idursulfase | Enzyme Replacement Agents | |
| Alglucosidase alpha | Enzyme Replacement Agents | CA2416492 |
| Exe tide | Hypoglycemic Agents | US6872700 |
| Mecasermin | | US5681814 |
| Pramlintide | | US5686411 |
| Galsulfase | Enzyme Replacement Agents | |
| Abatacept | Antirheumatic Agents and | CA2110518 |
| | Immunosuppressive Agents | |
| Cosyntropin | Hormones and Diagnostic Agents | |
| Corticotropin | | |
| Insulin aspart | Hypoglycemic Agents and | US5866538 |
| | Antidiabetic Agents | |
| Insulin detemir | Antidiabetic Agents | US5750497 |
| Insulin glulisine | Antidiabetic Agents | US6960561 |
| Pegaptanib | Intended for the prevention of | |
| | respiratory distress syndrome | |
| | (RDS) in premature infants at high | |
| | risk for RDS. | |
| Nesiritide | | |
| Thymalphasin | | |
| Defibrotide | Antithrombins | |
| tural alpha interferon OR | | |
| multiferon | | |
| Glatiramer acetate | | |
| Preotact | | |
| Teicoplanin | Anti-Bacterial Agents | |
| Ca kinumab | Anti-Inflammatory Agents and | |
| | Monoclo l antibodies | |
| Ipilimumab | Antineoplastic Agents and | CA2381770 |
| | Monoclo l antibodies | |
| Sulodexide | Antithrombins and Fibrinolytic | |
| | Agents and Hypoglycemic Agents | |
| | and Anticoagulants and | |
| | Hypolipidemic Agents | |
| Tocilizumab | | CA2201781 |
| Teriparatide | Bone Density Conservation | US6977077 |
| | Agents | |
| Pertuzumab | Monoclo l antibodies | CA2376596 |
| Rilo cept | Immunosuppressive Agents | US5844099 |
| Denosumab | Bone Density Conservation | CA2257247 |
| | Agents and Monoclo l antibodies | |
| Liraglutide | | US6268343 |
| Golimumab | Antipsoriatic Agents and Monoclo | |
| | l antibodies and TNF inhibitor | |
| Belatacept | Antirheumatic Agents and | |
| | Immunosuppressive Agents | |
| Buserelin | | |
| Velaglucerase alpha | Enzymes | US7138262 |
| Tesamorelin | | US5861379 |
| Brentuximab vedotin | | |
| Taliglucerase alpha | Enzymes | |
| Belimumab | Monoclo l antibodies | |
| Aflibercept | Antineoplastic Agents and | US7306799 |
| | Ophthalmics | |
| Asparagi se erwinia | Enzymes | |
| chrysanthemi | | |
| Ocriplasmin | Ophthalmics | |
| Glucarpidase | Enzymes | |
| Teduglutide | | US5789379 |
| Raxibacumab | Anti-Infective Agents and | |
| | Monoclo l antibodies | |
| Certolizumab pegol | TNF inhibitor | CA2380298 |
| Insulin, isophane | Hypoglycemic Agents and | |
| | Antidiabetic Agents | |
| Epoetin zeta | | |
| Obinutuzumab | Antineoplastic Agents | |
| Fibrinolysin aka plasmin | | US3234106 |
| Follitropin alpha | | |
| Romiplostim | Colony-Stimulating Factors and | |
| | Thrombopoietic Agents | |
| Luci ctant | Pulmo ry surfactants | US5407914 |
| talizumab | Immunosuppressive agents | |
| Aliskiren | Renin inhibitor | |
| Ragweed Pollen Extract | | |
| Secukinumab | Inhibitor | US20130202610 |
| Somatotropin Recombi nt | Hormone Replacement Agents | CA1326439 |
| Drotrecogin alpha | Antisepsis | CA2036894 |
| Alefacept | Dermatologic and | |
| | Immunosupressive agents | |
| OspA lipoprotein | Vaccines | |
| Uroki se | | US4258030 |
| Abarelix | Anti-Testosterone Agents | US5968895 |
| Sermorelin | Hormone Replacement Agents | |
| Aprotinin | | US5198534 |
| Gemtuzumab ozogamicin | Antineoplastic agents and | US5585089 |
| | Immunotoxins | |
| Satumomab Pendetide | Diagnostic Agents | |
| Albiglutide | Drugs used in diabetes; alimentary | |
| | tract and metabolism; blood | |
| | glucose lowering drugs, excl. | |
| | insulins. | |
| Alirocumab | | |
| Ancestim | | |
| Antithrombin alpha | | |
| Antithrombin III human | | |
| Asfotase alpha | Enzymes Alimentary Tract and | |
| | Metabolism | |
| Atezolizumab | | |
| Autologous cultured | | |
| chondrocytes | | |
| Beractant | | |
| Bli tumomab | Antineoplastic Agents | US20120328618 |
| | Immunosuppressive Agents | |
| | Monoclo l antibodies | |
| | Antineoplastic and | |
| | Immunomodulating Agents | |
| C1 Esterase Inhibitor | | |
| (Human) | | |
| Coagulation Factor XIII | | |
| A-Subunit (Recombi nt) | | |
| Conestat alpha | | |
| Daratumumab | Antineoplastic Agents | |
| Desirudin | | |
| Dulaglutide | Hypoglycemic Agents; Drugs | |
| | Used in Diabetes; Alimentary | |
| | Tract and Metabolism; Blood | |
| | Glucose Lowering Drugs, Excl. | |
| | Insulins | |
| Elosulfase alpha | Enzymes; Alimentary Tract and | |
| | Metabolism | |
| Elotuzumab | | US2014055370 |
| Evolocumab | Lipid Modifying Agents, Plain; | |
| | Cardiovascular System | |
| Fibrinogen Concentrate | | |
| (Human) | | |
| Filgrastim-sndz | | |
| Gastric intrinsic factor | | |
| Hepatitis B immune | | |
| globulin | | |
| Human calcitonin | | |
| Human clostridium tetani | | |
| toxoid immune globulin | | |
| Human rabies virus | | |
| immune globulin | | |
| Human Rho(D) immune | | |
| globulin | | |
| Hyaluronidase (Human | | US7767429 |
| Recombi nt) | | |
| Idarucizumab | Anticoagulant | |
| Immune Globulin Human | Immunologic Factors; | |
| | Immunosuppressive Agents; | |
| | Anti-Infective Agents | |
| Vedolizumab | Immunosupressive agent, | US2012151248 |
| | Antineoplastic agent | |
| Ustekinumab | Deramtologic agent, | |
| | Immunosuppressive agent, | |
| | antineoplastic agent | |
| Turoctocog alpha | | |
| Tuberculin Purified Protein | | |
| Derivative | | |
| Simoctocog alpha | Antihaemorrhagics: blood | |
| | coagulation factor VIII | |
| Siltuximab | Antineoplastic and | US7612182 |
| | Immunomodulating Agents, | |
| | Immunosuppressive Agents | |
| Sebelipase alpha | Enzymes | |
| Sacrosidase | Enzymes | |
| Ramucirumab | Antineoplastic and | US2013067098 |
| | Immunomodulating Agents | |
| Prothrombin complex | | |
| concentrate | | |
| Poractant alpha | Pulmo ry Surfactants | |
| Pembrolizumab | Antineoplastic and | US2012135408 |
| | Immunomodulating Agents | |
| Peginterferon beta-1a | | |
| Ofatumumab | Antineoplastic and | US8337847 |
| | Immunomodulating Agents | |
| Obiltoxaximab | | |
| Nivolumab | Antineoplastic and | US2013173223 |
| | Immunomodulating Agents | |
| Necitumumab | | |
| Metreleptin | | US20070099836 |
| Methoxy polyethylene | | |
| glycol-epoetin beta | | |
| Mepolizumab | Antineoplastic and | US2008134721 |
| | Immunomodulating Agents, | |
| | Immunosuppressive Agents, | |
| | Interleukin Inhibitors | |
| Ixekizumab | | |
| Insulin Pork | Hypoglycemic Agents, | |
| | Antidiabetic Agents | |
| Insulin Degludec | | |
| Insulin Beef | | |
| Thyroglobulin | Hormone therapy | US5099001 |
| Anthrax immune globulin | Plasma derivative | |
| human | | |
| Anti-inhibitor coagulant | Blood Coagulation Factors, | |
| complex | Antihemophilic Agent | |
| Anti-thymocyte Globulin | Antibody | |
| (Equine) | | |
| Anti-thymocyte Globulin | Antibody | |
| (Rabbit) | | |
| Brodalumab | Antineoplastic and | |
| | Immunomodulating Agents | |
| C1 Esterase Inhibitor | Blood and Blood Forming Organs | |
| (Recombi nt) | | |
| Ca kinumab | Antineoplastic and | |
| | Immunomodulating Agents | |
| Chorionic Go dotropin | Hormones | US6706681 |
| (Human) | | |
| Chorionic Go dotropin | Hormones | US5767251 |
| (Recombi nt) | | |
| Coagulation factor X | Blood Coagulation Factors | |
| human | | |
| Dinutuximab | Antibody, Immunosuppresive | US20140170155 |
| | agent, Antineoplastic agent | |
| Efmoroctocog alpha | Antihemophilic Factor | |
| Factor IX Complex | Antihemophilic agent | |
| (Human) | | |
| Hepatitis A Vaccine | Vaccine | |
| Human Varicella-Zoster | Antibody | |
| Immune Globulin | | |
| Ibritumomab tiuxetan | Antibody, Immunosuppressive | CA2149329 |
| | Agents | |
| Lenograstim | Antineoplastic and | |
| | Immunomodulating Agents | |
| Pegloticase | Enzymes | |
| Protamine sulfate | Heparin Antagonists, Hematologic | |
| | Agents | |
| Protein S human | Anticoagulant plasma protein | |
| Sipuleucel-T | Antineoplastic and | US8153120 |
| | Immunomodulating Agents | |
| Somatropin recombi nt | Hormones, Hormone Substitutes, | CA1326439, CA2252535, |
| | and Hormone Antagonists | US5288703, US5849700, |
| | | US5849704, US5898030, |
| | | US6004297, US6152897, |
| | | US6235004, US6899699 |
| Susoctocog alpha | Blood coagulation factors, | |
| | Antihaemorrhagics | |
| Thrombomodulin alpha | Anticoagulant agent, Antiplatelet | |
| | agent | |

TABLE 15
Exemplary monoclonal antibody therapies.

| mAb | Target | Indication |
|---|---|---|
| Muromonab- | CD3 | Kidney transplant rejection |
| CD3 | | |
| Abciximab | GPIIb/IIIa | Prevention of blood clots in |
| | | angioplasty |
| Rituximab | CD20 | Non-Hodgkin lymphoma |
| Palivizumab | RSV | Prevention of respiratory |
| | | syncytial virus infection |
| Infliximab | TNFα | Crohn's disease |
| Trastuzumab | HER2 | Breast cancer |
| Alemtuzumab | CD52 | Chronic myeloid leukemia |
| Adalimumab | TNFα | Rheumatoid arthritis |
| Ibritumomab | CD20 | Non-Hodgkin lymphoma |
| tiuxetan | | |
| Omalizumab | IgE | Asthma |
| Cetuximab | EGFR | Colorectal cancer |
| Bevacizumab | VEGF-A | Colorectal cancer |
| Natalizumab | ITGA4 | Multiple sclerosis |
| Panitumumab | EGFR | Colorectal cancer |
| Ranibizumab | VEGF-A | Macular degeneration |
| Eculizumab | C5 | Paroxysmal nocturnal hemoglobinuria |
| Certolizumab | TNFα | Crohn's disease |
| pegol | | |
| Ustekinumab | IL-12/23 | Psoriasis |
| Canakinumab | IL-1β | Muckle-Wells syndrome |
| Golimumab | TNFα | Rheumatoid and psoriatic arthritis, |
| | | ankylosing spondylitis |
| Ofatumumab | CD20 | Chronic lymphocytic leukemia |
| Tocilizumab | IL-6R | Rheumatoid arthritis |
| Denosumab | RANKL | Bone loss |
| Belimumab | BLyS | Systemic lupus erythematosus |
| Ipilimumab | CTLA-4 | Metastatic melanoma |
| Brentuximab | CD30 | Hodgkin lymphoma, systemic |
| vedotin | | anaplastic large cell lymphoma |
| Pertuzumab | HER2 | Breast Cancer |
| Trastuzumab | HER2 | Breast cancer |
| emtansine | | |
| Raxibacumab | B. anthrasis | Anthrax infection |
| | PA | |
| Obinutuzumab | CD20 | Chronic lymphocytic leukemia |
| Siltuximab | IL-6 | Castleman disease |
| Ramucirumab | VEGFR2 | Gastric cancer |
| Vedolizumab | α4β7 integrin | Ulcerative colitis, Crohn disease |
| Blinatumomab | CD19, CD3 | Acute lymphoblastic leukemia |
| Nivolumab | PD-1 | Melanoma, non-small cell lung |
| | | cancer |
| Pembrolizumab | PD-1 | Melanoma |
| Idarucizumab | Dabigatran | Reversal of dabigatran-induced |
| | | anticoagulation |
| Necitumumab | EGFR | Non-small cell lung cancer |
| Dinutuximab | GD2 | Neuroblastoma |
| Secukinumab | IL-17α | Psoriasis |
| Mepolizumab | IL-5 | Severe eosinophilic asthma |
| Alirocumab | PCSK9 | High cholesterol |
| Evolocumab | PCSK9 | High cholesterol |
| Daratumumab | CD38 | Multiple myeloma |
| Elotuzumab | SLAMF7 | Multiple myeloma |
| Ixekizumab | IL-17α | Psoriasis |
| Reslizumab | IL-5 | Asthma |
| Olaratumab | PDGFRα | Soft tissue sarcoma |
| Bezlotoxumab | Clostridium | Prevention of Clostridium |
| | difficile | difficile |
| | enterotoxin B | infection recurrence |
| Atezolizumab | PD-L1 | Bladder cancer |
| Obiltoxaximab | B. anthrasis | Prevention of inhalational anthrax |
| | PA | |
| Inotuzumab | CD22 | Acute lymphoblastic leukemia |
| ozogamicin | | |
| Brodalumab | IL-17R | Plaque psoriasis |
| Guselkumab | IL-23 p19 | Plaque psoriasis |
| Dupilumab | IL-4Rα | Atopic dermatitis |
| Sarilumab | IL-6R | Rheumatoid arthritis |
| Avelumab | PD-L1 | Merkel cell carcinoma |
| Ocrelizumab | CD20 | Multiple sclerosis |
| Emicizumab | Factor IXa, X | Hemophilia A |
| Benralizumab | IL-5Rα | Asthma |
| Gemtuzumab | CD33 | Acute myeloid leukemia |
| ozogamicin | | |
| Durvalumab | PD-L1 | Bladder cancer |
| Burosumab | FGF23 | X-linked hypophosphatemia |
| Lanadelumab | Plasma | Hereditary angioedema attacks |
| | kallikrein | |
| Mogamulizumab | CCR4 | Mycosis fungoides or Sézary |
| | | syndrome |
| Erenumab | CGRPR | Migraine prevention |
| Galcanezumab | CGRP | Migraine prevention |
| Tildrakizumab | IL-23 p19 | Plaque psoriasis |
| Cemiplimab | PD-1 | Cutaneous squamous cell |
| | | carcinoma |
| Emapalumab | IFNγ | Primary hemophagocytic |
| | | lymphohistiocytosis |
| Fremanezumab | CGRP | Migraine prevention |
| Ibalizumab | CD4 | HIV infection |
| Moxetumomab | CD22 | Hairy cell leukemia |
| pasudodox | | |
| Ravulizumab | C5 | Paroxysmal nocturnal |
| | | hemoglobinuria |
| Caplacizumab | von Willebrand | Acquired thrombotic |
| | factor | thrombocytopeniarpura |
| Romosozumab | Sclerostin | Osteoporosis in postmenopausal |
| | | women at increased risk of |
| | | fracture |
| Risankizumab | IL-23 p19 | Plaque psoriasis |
| Polatuzumab | CD79β | Diffuse large B-cell lymphoma |
| vedotin | | |
| Brolucizumab | VEGF-A | Macular degeneration |
| Crizanlizumab | P-selectin | Sickle cell disease |

### APPLICATIONS

Using the systems described herein, optionally using any of delivery modalities described herein (including nanoparticle delivery modalities, such as lipid nanoparticles, and viral delivery modalities, such as AAVs), the invention also provides applications (methods) for modifying a DNA molecule, such as nuclear DNA, i.e., in the genome of a cell, whether in vitro, ex vivo, in situ, or in vivo, e.g., in a tissue in an organism, such as a subject including mammalian subjects, such as a human. By integrating coding genes into a DNA sequence template, the Gene Writer system can address therapeutic needs, for example, by providing expression of a therapeutic transgene (e.g., comprised in an object sequence as described herein) in individuals with loss-of-function mutations, by replacing gain-of-function mutations with normal transgenes, by providing regulatory sequences to eliminate gain-of-function mutation expression, and/or by controlling the expression of operably linked genes, transgenes and systems thereof. In certain embodiments, an object sequence (e.g., a heterologous object sequence) comprises a coding sequence encoding a functional element (e.g., a polypeptide or non-coding RNA, e.g., as described herein) specific to the therapeutic needs of the host cell. In some embodiments, an object sequence (e.g., a heterologous object sequence) comprises a promoter, for example, a tissue specific promotor or enhancer. In some embodiments, a promotor can be operably linked to a coding sequence.

In certain aspects, the invention this provides methods of modifying a target DNA strand in a cell, tissue or subject, comprising administering a system as described herein (optionally by a modality described herein) to the cell, tissue or subject, where the system inserts the heterologous object sequence into the target DNA strand, thereby modifying the target DNA strand. In certain embodiments, the heterologous object sequence is thus expressed in the cell, tissue, or subject. In some embodiments, the cell, tissue or subject is a mammalian (e.g., human) cell, tissue or subject. Exemplary cells thus modified include a hepatocyte, lung epithelium, an ionocyte. Such a cell may be a primary cell or otherwise not immortalized. In related aspects, the invention also provides methods of treating a mammalian tissue comprising administering a system as described herein to the mammal, thereby treating the tissue, wherein the tissue is deficient in the heterologous object sequence. In certain embodiments of any of the foregoing aspects and embodiments, the Gene Writer polypeptide is provided as a nucleic acid, which is present transiently.

In some embodiments, a system of the invention is capable of producing an insertion in target DNA. It is conceived that the systems described herein are capable of resulting in the expression of an exogenous non-coding nucleic acid, e.g., miRNA, lncRNA, shRNA, siRNA, tRNA, mtRNA, gRNA, or rRNA, expression of a protein coding sequence, e.g., a therapeutic protein or a regulatory protein, incorporation of a regulatory element, e.g., a promoter, enhancer, transcription factor binding site, epigenetic modifier site, miRNA binding site, splice donor or acceptor site, or a terminator sequence, or incorporation of other DNA sequence, e.g., spacer. Depending on the content and context of the insertion, it is thus possible to express an exogenous protein or alter expression of an endogenous protein or cellular system. In some embodiments, a Gene Writing system may be used to knockout an endogenous gene by insertional mutagenesis, e.g., by integration of an insert DNA into a coding or regulatory region. In some embodiments, a Gene Writing system may be used to simultaneously trigger expression of a transgene cassette, e.g., a CAR, while disrupting expression of an endogenous gene or locus, e.g., TRAC, by mediating integration of an insert DNA encoding the transgene cassette into the endogenous gene or locus. In some embodiments, a Gene Writing system may be used to substitute an allele by integrating a transgene expression cassette into the endogenous allele, thus disrupting its expression.

In embodiments, the Gene Writer™ gene editor system can provide an object sequence comprising, e.g., a therapeutic agent (e.g., a therapeutic transgene) expressing, e.g., replacement blood factors or replacement enzymes, e.g., lysosomal enzymes. For example, the compositions, systems and methods described herein are useful to express, in a target human genome, agalsidase alpha or beta for treatment of Fabry Disease; imiglucerase, taliglucerase alfa, velaglucerase alfa, or alglucerase for Gaucher Disease; sebelipase alpha for lysosomal acid lipase deficiency (Wolman disease/CESD); laronidase, idursulfase, elosulfase alpha, or galsulfase for mucopolysaccharidoses; alglucosidase alpha for Pompe disease. For example, the compositions, systems and methods described herein are useful to express, in a target human genome factor I, II, V, VII, X, XI, XII or XIII for blood factor deficiencies.

In some embodiments, the heterologous object sequence encodes an intracellular protein (e.g., a cytoplasmic protein, a nuclear protein, an organellar protein such as a mitochondrial protein or lysosomal protein, or a membrane protein). In some embodiments, the heterologous object sequence encodes a membrane protein, e.g., a membrane protein other than a CAR, and/or an endogenous human membrane protein. In some embodiments, the heterologous object sequence encodes an extracellular protein. In some embodiments, the heterologous object sequence encodes an enzyme, a structural protein, a signaling protein, a regulatory protein, a transport protein, a sensory protein, a motor protein, a defense protein, or a storage protein. Other proteins include an immune receptor protein, e.g. a synthetic immune receptor protein such as a chimeric antigen receptor protein (CAR), a T cell receptor, a B cell receptor, or an antibody.

A Gene Writing™ system may be used to modify immune cells. In some embodiments, a Gene Writing™ system may be used to modify T cells. In some embodiments, T-cells may include any subpopulation of T-cells, e.g., CD4+, CD8+, gamma-delta, naïve T cells, stem cell memory T cells, central memory T cells, or a mixture of subpopulations. In some embodiments, a Gene Writing™ system may be used to deliver or modify a T-cell receptor (TCR) in a T cell. In some embodiments, a Gene Writing™ system may be used to deliver at least one chimeric antigen receptor (CAR) to T-cells. In some embodiments, a Gene Writing™ system may be used to deliver at least one CAR to natural killer (NK) cells. In some embodiments, a Gene Writing™ system may be used to deliver at least one CAR to natural killer T (NKT) cells. In some embodiments, a Gene Writing™ system may be used to deliver at least one CAR to a progenitor cell, e.g., a progenitor cell of T, NK, or NKT cells. In some embodiments, cells modified with at least one CAR (e.g., CAR-T cells, CAR-NK cells, CAR-NKT cells), or a combination of cells modified with at least one CAR (e.g., a mixture of CAR-NK/T cells) are used to treat a condition as identified in the targetable landscape of CAR therapies in MacKay, et al. Nat Biotechnol 38, 233-244 (2020), incorporated by reference herein in its entirety. In some embodiments, the immune cells comprise a CAR specific to a tumor or a pathogen antigen selected from a group consisting of AChR (fetal acetylcholine receptor), ADGRE2, AFP (alpha fetoprotein), BAFF-R, BCMA, CAIX (carbonic anhydrase IX), CCR1, CCR4, CEA (carcinoembryonic antigen), CD3, CD5, CD8, CD7, CD10, CD13, CD14, CD15, CD19, CD20, CD22, CD30, CD33, CLLI, CD34, CD38, CD41, CD44, CD49f, CD56, CD61, CD64, CD68, CD70,CD74, CD99,CD117, CD123, CD133, CD138, CD44v6, CD267, CD269, CDS, CLEC12A, CS1, EGP-2 (epithelial glycoprotein-2), EGP-40 (epithelial glycoprotein-40), EGFR(HER1), EGFR-VIII, EpCAM (epithelial cell adhesion molecule), EphA2, ERBB2 (HER2, human epidermal growth factor receptor 2), ERBB3, ERBB4, FBP (folate-binding protein), Flt3 receptor, folate receptor-a, GD2 (ganglioside G2), GD3 (ganglioside G3), GPC3 (glypican-3), GPI00, hTERT (human telomerase reverse transcriptase), ICAM-1, integrin B7, interleukin 6 receptor, IL13Ra2 (interleukin-13 receptor 30 subunit alpha-2), kappa-light chain, KDR (kinase insert domain receptor), LeY (Lewis Y), L1CAM (LI cell adhesion molecule), LILRB2 (leukocyte immunoglobulin like receptor B2), MARTI, MAGE-A1 (melanoma associated antigen A1), MAGE-A3, MSLN (mesothelin), MUC16 (mucin 16), MUCI (mucin I), KG2D ligands, NY-ESO-1 (cancer-testis antigen), PRI (proteinase 3), TRBCI, TRBC2, TFM-3, TACI, tyrosinase, survivin, hTERT, oncofetal antigen (h5T4), p53, PSCA (prostate stem cell antigen), PSMA (prostate-specific membrane antigen), hRORI, TAG-72 (tumor-associated glycoprotein 72), VEGF-R2 (vascular endothelial growth factor R2), WT-1 (Wilms tumor protein), and antigens of HIV (human immunodeficiency virus), hepatitis B, hepatitis C, CMV (cytomegalovirus), EBV (Epstein-Barr virus), HPV (human papilloma virus).

In some embodiments, immune cells, e.g., T-cells, NK cells, NKT cells, or progenitor cells are modified ex vivo and then delivered to a patient. In some embodiments, a Gene Writer™ system is delivered by one of the methods mentioned herein, and immune cells, e.g., T-cells, NK cells, NKT cells, or progenitor cells are modified in vivo in the patient.

In some embodiments, a Gene Writing system can be used to make multiple modifications to a target cell, either simultaneously or sequentially. In some embodiments, a Gene Writing system can be used to further modify an already modified cell. In some embodiments, a Gene Writing system can be use to modify a cell edited by a complementary technology, e.g., a gene edited cell, e.g., a cell with one or more CRISPR knockouts. In some embodiments, the previously edited cell is a T-cell. In some embodiments, the previous modifications comprise gene knockouts in a T-cell, e.g., endogenous TCR (e.g., TRAC, TRBC), HLA Class I (B2M), PD1, CD52, CTLA-4, TIM-3, LAG-3, DGK. In some embodiments, a Gene Writing system is used to insert a TCR or CAR into a T-cell that has been previously modified.

### Administration

The composition and systems described herein may be used in vitro or in vivo. In some embodiments the system or components of the system are delivered to cells (e.g., mammalian cells, e.g., human cells), e.g., in vitro or in vivo. The skilled artisan will understand that the components of the Gene Writer system may be delivered in the form of polypeptide, nucleic acid (e.g., DNA, RNA), and combinations thereof.

In some embodiments, the system and/or components of the system are delivered as nucleic acids. For example, the recombinase polypeptide may be delivered in the form of a DNA or RNA encoding the recombinase polypeptide. In some embodiments the system or components of the system (e.g., an insert DNA and a recombinase polypeptide-encoding nucleic acid molecule) are delivered on 1, 2, 3, 4, or more distinct nucleic acid molecules. In some embodiments the system or components of the system are delivered as a combination of DNA and RNA. In some embodiments the system or components of the system are delivered as a combination of DNA and protein. In some embodiments the system or components of the system are delivered as a combination of RNA and protein. In some embodiments the recombinase polypeptide is delivered as a protein.

In some embodiments the system or components of the system are delivered to cells, e.g. mammalian cells or human cells, using a vector. The vector may be, e.g., a plasmid or a virus. In some embodiments delivery is in vivo, in vitro, ex vivo, or in situ. In some embodiments the virus is an adeno associated virus (AAV), a lentivirus, an adenovirus. In some embodiments the system or components of the system are delivered to cells with a viral-like particle or a virosome. In some embodiments the delivery uses more than one virus, viral-like particle or virosome.

In some embodiments, the recombinase is active upon linear or circular single or double stranded DNA. In some embodiments, the recombinase is active upon DNA after it is converted from single stranded to double stranded in the cell. In some embodiments, the recombinase is active upon DNA after it has formed a concatemer in the cell. In some embodiments, the recombinase polypeptide is delivered to or expressed in the cell after the insert DNA is converted from single to double stranded.

In some embodiments, recombinase recognition sequences are present 5′ and 3′ of the nucleic acid encoding the recombinase polypeptide. In some embodiments, the recombinase recognition sequences are an attB and an attP with compatible spacer regions and central dinucleotides. In some embodiments, the recombinase recognition sequences have a different spacer region and/or central dinucleotide than the recombinase recognition sequences on the insert DNA or at a target site in the genome. In some embodiments, the recombinase recognition sites do not interact with the recombinase recognition sites on the insert DNA or in the genome. In some embodiments the recombinase recognition sequences are directly adjacent to the nucleic acid encoding the open reading frame of the recombinase polypeptide. In some embodiments the recombinase recognition sequences are external to a gene expression unit for the recombinase. In some embodiments the recombinase recognition sequences (e.g. attB and attP) are in the same 5′ to 3′ orientation. In some embodiments the recombinase recognition sequences (e.g. attB and attP) are in the opposite 5′ to 3′ orientation. In some embodiments, the recombinase polypeptide recombines the recognition sequences that are 5′ and 3′ of the nucleic acid encoding the recombinase polypeptide, resulting in a decrease of recombinase gene expression.

In some embodiments, multiple recombinase recognition sequences are present on the insert DNA. In some embodiments, the insert DNA comprises two or more recognition sequences. In some embodiments, the insert DNA comprises three or more recognition sequences. In some embodiments, the insert DNA comprises two recognition sequences (e.g. an attB and attP) that are compatible with each other, and a third recognition sequence (e.g. an attB or an attP) that is incompatible with the other recognition sequences on the insert DNA. In some embodiments, the recognition sequences on the insert DNA that are compatible with each other are not compatible with recognition sequences in the target genome. In some embodiments, the recognition sequence on the insert DNA that is incompatible with the other recognition sequences on the insert DNA is compatible with recognition sequences in the target genome. In some embodiments the recognition sequences that are compatible with each other have compatible spacer regions and central dinucleotides, and the recognition sequences that are incompatible have incompatible spacer regions and central dinucleotides. In some embodiments, the compatible recognition sequences on the insert DNA are in the same 5′ to 3′ orientation. In some embodiments, the recombinase acts upon the compatible recognition sequences on the insert DNA to form a circular DNA. In some embodiments, the resulting circular DNA comprises an attL, attR, and either an attP or attB sequence, wherein the attP or attB sequence is compatible with recognition sequences in the target genome. In some embodiments, the multiple recombinase recognition sequences described herein are present in a viral vector genome.

In one embodiment, the compositions and systems described herein can be formulated in liposomes or other similar vesicles. Liposomes are spherical vesicle structures composed of a uni- or multilamellar lipid bilayer surrounding internal aqueous compartments and a relatively impermeable outer lipophilic phospholipid bilayer. Liposomes may be anionic, neutral or cationic. Liposomes are biocompatible, nontoxic, can deliver both hydrophilic and lipophilic drug molecules, protect their cargo from degradation by plasma enzymes, and transport their load across biological membranes and the blood brain barrier (BBB) (see, e.g., Spuch and Navarro, Journal of Drug Delivery, vol. 2011, Article ID 469679, 12 pages, 2011. doi:10.1155/2011/469679 for review).

Vesicles can be made from several different types of lipids; however, phospholipids are most commonly used to generate liposomes as drug carriers. Methods for preparation of multilamellar vesicle lipids are known in the art (see for example U.S. Pat. No. 6,693,086, the teachings of which relating to multilamellar vesicle lipid preparation are incorporated herein by reference). Although vesicle formation can be spontaneous when a lipid film is mixed with an aqueous solution, it can also be expedited by applying force in the form of shaking by using a homogenizer, sonicator, or an extrusion apparatus (see, e.g., Spuch and Navarro, Journal of Drug Delivery, vol. 2011, Article ID 469679, 12 pages, 2011. doi:10.1155/2011/469679 for review). Extruded lipids can be prepared by extruding through filters of decreasing size, as described in Templeton et al., Nature Biotech, 15:647-652, 1997, the teachings of which relating to extruded lipid preparation are incorporated herein by reference.

Lipid nanoparticles are another example of a carrier that provides a biocompatible and biodegradable delivery system for the pharmaceutical compositions described herein. Nanostructured lipid carriers (NLCs) are modified solid lipid nanoparticles (SLNs) that retain the characteristics of the SLN, improve drug stability and loading capacity, and prevent drug leakage. Polymer nanoparticles (PNPs) are an important component of drug delivery. These nanoparticles can effectively direct drug delivery to specific targets and improve drug stability and controlled drug release. Lipid-polymer nanoparticles (PLNs), a new type of carrier that combines liposomes and polymers, may also be employed. These nanoparticles possess the complementary advantages of PNPs and liposomes. A PLN is composed of a core-shell structure; the polymer core provides a stable structure, and the phospholipid shell offers good biocompatibility. As such, the two components increase the drug encapsulation efficiency rate, facilitate surface modification, and prevent leakage of water-soluble drugs. For a review, see, e.g., Li et al. 2017, Nanomaterials 7, 122; doi:10.3390/nano7060122.

Exosomes can also be used as drug delivery vehicles for the compositions and systems described herein. For a review, see Ha et al. July 2016. Acta Pharmaceutica Sinica B. Volume 6, Issue 4, Pages 287-296; https://doi.org/10.1016/j.apsb.2016.02.001.

In some embodiments, at least one component of a system described herein comprises a fusosome. Fusosomes interact and fuse with target cells, and thus can be used as delivery vehicles for a variety of molecules. They generally consist of a bilayer of amphipathic lipids enclosing a lumen or cavity and a fusogen that interacts with the amphipathic lipid bilayer. The fusogen component has been shown to be engineerable in order to confer target cell specificity for the fusion and payload delivery, allowing the creation of delivery vehicles with programmable cell specificity (see, for example, the sections relating to fusosome design, preparation, and usage in PCT Publication No. WO/2020014209, incorporated herein by reference in its entirety).

A Gene Writer system can be introduced into cells, tissues and multicellular organisms. In some embodiments the system or components of the system are delivered to the cells via mechanical means or physical means.

Formulation of protein therapeutics is described in Meyer (Ed.), Therapeutic Protein Drug Products: Practical Approaches to formulation in the Laboratory, Manufacturing, and the Clinic, Woodhead Publishing Series (2012).

In some embodiments, a Gene Writer™ system described herein is delivered to a tissue or cell from the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type. In some embodiments, a Gene Writer™ system described herein is used to treat a disease, such as a cancer, inflammatory disease, infectious disease, genetic defect, or other disease. A cancer can be cancer of the cerebrum, cerebellum, adrenal gland, ovary, pancreas, parathyroid gland, hypophysis, testis, thyroid gland, breast, spleen, tonsil, thymus, lymph node, bone marrow, lung, cardiac muscle, esophagus, stomach, small intestine, colon, liver, salivary gland, kidney, prostate, blood, or other cell or tissue type, and can include multiple cancers.

In some embodiments, a Gene Writer™ system described herein described herein is administered by enteral administration (e.g. oral, rectal, gastrointestinal, sublingual, sublabial, or buccal administration). In some embodiments, a Gene Writer™ system described herein is administered by parenteral administration (e.g., intravenous, intramuscular, subcutaneous, intradermal, epidural, intracerebral, intracerebroventricular, epicutaneous, nasal, intra-arterial, intra-articular, intracavernous, intraocular, intraosseous infusion, intraperitoneal, intrathecal, intrauterine, intravaginal, intravesical, perivascular, or transmucosal administration). In some embodiments, a Gene Writer™ system described herein is administered by topical administration (e.g., transdermal administration).

In some embodiments, a Gene Writer™ system as described herein can be used to modify an animal cell, plant cell, or fungal cell. In some embodiments, a Gene Writer™ system as described herein can be used to modify a mammalian cell (e.g., a human cell). In some embodiments, a Gene Writer™ system as described herein can be used to modify a cell from a livestock animal (e.g., a cow, horse, sheep, goat, pig, llama, alpaca, camel, yak, chicken, duck, goose, or ostrich). In some embodiments, a Gene Writer™ system as described herein can be used as a laboratory tool or a research tool, or used in a laboratory method or research method, e.g., to modify an animal cell, e.g., a mammalian cell (e.g., a human cell), a plant cell, or a fungal cell.

In some embodiments, a Gene Writer™ system as described herein can be used to express a protein, template, or heterologous object sequence (e.g., in an animal cell, e.g., a mammalian cell (e.g., a human cell), a plant cell, or a fungal cell). In some embodiments, a Gene Writer™ system as described herein can be used to express a protein, template, or heterologous object sequence under the control of an inducible promoter (e.g., a small molecule inducible promoter). In some embodiments, a Gene Writing system or payload thereof is designed for tunable control, e.g., by the use of an inducible promoter. For example, a promoter, e.g., Tet, driving a gene of interest may be silent at integration, but may, in some instances, activated upon exposure to a small molecule inducer, e.g., doxycycline. In some embodiments, the tunable expression allows post-treatment control of a gene (e.g., a therapeutic gene), e.g., permitting a small molecule-dependent dosing effect. In embodiments, the small molecule-dependent dosing effect comprises altering levels of the gene product temporally and/or spatially, e.g., by local administration. In some embodiments, a promoter used in a system described herein may be inducible, e.g., responsive to an endogenous molecule of the host and/or an exogenous small molecule administered thereto.

### Treatment of Suitable Indications

In some embodiments, a Gene Writer™ system described herein, or a component or portion thereof (e.g., a polypeptide or nucleic acid as described herein), is used to treat a disease, disorder, or condition. In some embodiments, the Gene Writer™ system described herein, or component or portion thereof, is used to treat a disease, disorder, or condition listed in any of Tables 16-21. In some embodiments, the Gene Writer™ system described herein, or component or portion thereof, is used to treat a hematopoietic stem cell (HSC) disease, disorder, or condition, e.g., as listed in Table 16. In some embodiments, the Gene Writer™ system described herein, or component or portion thereof, is used to treat a kidney disease, disorder, or condition, e.g., as listed in Table 17. In some embodiments, the Gene Writer™ system described herein, or component or portion thereof, is used to treat a liver disease, disorder, or condition, e.g., as listed in Table 18. In some embodiments, the Gene Writer™ system described herein, or component or portion thereof, is used to treat a lung disease, disorder, or condition, e.g., as listed in Table 19. In some embodiments, the Gene Writer™ system described herein, or component or portion thereof, is used to treat a skeletal muscle disease, disorder, or condition, e.g., as listed in Table 20. In some embodiments, the Gene Writer™ system described herein, or component or portion thereof, is used to treat a skin disease, disorder, or condition, e.g., as listed in Table 21.

### Tables 16-21: Indications Selected for Trans Gene Writers to be Used for Recombinases

TABLE 16
HSCs

| Disease | Gene Affected |
|---|---|
| Adrenoleukodystrophy (CALD) | ABCD1 |
| Alpha-mannosidosis | MAN2B1 |
| Fanconi anemia | FANCA; FANCC; FANCG |
| Gaucher disease | GBA |
| Globoid cell leukodystrophy (Krabbe | GALC |
| disease) | |
| Hemophagocytic lymphohistiocytosis | PRF1; STX11; STXBP2; |
| | UNC13D |
| Malignant infantile osteopetrosis- | TCIRG1; Many genes |
| autosomal recessive osteopetrosis | implicated |
| Metachromatic leukodystrophy | ARSA; PSAP |
| MPS 1S (Scheie syndrome) | IDUA |
| MPS2 | IDS |
| MPS7 | GUSB |
| Mucolipidosis II | GNPTAB |
| Niemann-Pick disease A and B | SMPD1 |
| Niemann-Pick disease C | NPC1 |
| Pompe disease | GAA |
| Sickle cell disease (SCD) | HBB |
| Tay Sachs | HEXA |
| Thalassemia | HBB |

TABLE 17
Kidney

| Disease | Gene Affected |
|---|---|
| Congenital nephrotic syndrome | NPHS2 |
| Cystinosis | CTNS |

TABLE 18
Liver

| Disease | Gene Affected |
|---|---|
| Acute intermittent porphyria | HMBS |
| Alagille syndrome | JAG1 |
| Alpha-1 antitrypsin deficiency | SERPINA1 |
| Carbamoyl phosphate synthetase | CPS1 |
| I deficiency | |
| Citrullinemia I | ASS1 |
| Crigler-Najjar | UGT1A1 |
| Fabry | LPL |
| Familial chylomicronemia syndrome | GLA |
| Gaucher | GBE1 |
| GSD1a | G6Pase |
| GSD IV | GBA |
| Heme A | F8 |
| Heme B | F9 |
| HoFH | LDLRAP1 |
| Methylmalonic acidemia | MMUT |
| MPS II | IDS |
| MPS III | Type IIIa: SGSH |
| | Type IIIb: NAGLU |
| | Type IIIc: HGSNAT |
| | Type IIId: GNS S |
| MPS IV | Type IVA: GALNS |
| | Type IVB: GLB1 |
| MPS VI | ARSB |
| MSUD | Type Ia: BCKDHA |
| | Type Ib: BCKDHB |
| | Type II: DBT |
| OTC Deficiency | OTC |
| Polycystic Liver Disease | PRKCSH |
| Pompe | GAA |
| Primary Hyperoxaluria 1 | AGXT (HAO1 or LDHA |
| | for CRISPR) |
| Progressive familial intrahepatic | ATP8B1 |
| cholestasis type 1 | |
| Progressive familial intrahepatic | ABCB11 |
| cholestasis type 2 | |
| Progressive familial intrahepatic | ABCB4 |
| cholestasis type 3 | |
| Propionic acidemia | PCCB; PCCA |
| Wilson's Disease | ATP7B |

TABLE 19
Lung

| Disease | Gene Affected |
|---|---|
| Alpha-1 antitrypsin deficiency | SERPINA1 |
| Cystic fibrosis | CFTR |
| Primary ciliary dyskinesia | DNAI1 |
| Primary ciliary dyskinesia | DNAH5 |
| Primary pulmonary hypertension I | BMPR2 |
| Surfactant Protein B (SP-B) Deficiency | SFTPB |
| (pulmonary surfactant metabolism dysfunction 1) | |

TABLE 20
Skeletal muscle

| Disease | Gene Affected |
|---|---|
| Becker muscular dystrophy | DMD |
| Becker myotonia | CLCN1 |
| Bethlem myopathy | COL6A2 |
| Centronuclear myopathy, X-linked (motubular) | MTM1 |
| Congenital myasthenic syndrome | CHRNE |
| Duchenne muscular dystrophy | DMD |
| Emery-Dreifuss muscular dystrophy, AD | LMNA |
| Limb-girdle muscular dystrophy 2A | CAPN3 |
| Limb-girdle muscular dystrophy, type 2D | SGCA |

TABLE 21
Skin

| Disease | Gene Affected |
|---|---|
| Epidermolysis Bullosa Dystrophica Recessive | COL7A1 |
| (Hallopeau-Siemens) | |
| Epidermolysis Bullosa Junctional | LAMB3 |
| Epidermolytic Ichthyosis | KRT1; KRT10 |
| Hailey-Hailey Disease | ATP2C1 |
| Lamellar Ichthyosis/Nonbullous Congenital | TGM1 |
| Ichthyosiform Erythroderma (ARCI) | |
| Netherton Syndrome | SPINK5 |

In some embodiments, a Gene Writing system may be used to treat a healthy individual, e.g., as a preventative therapy. Gene Writing systems can, in some embodiments, be targeted to generate mutations, e.g., knockout mutations, that have been shown to be protective towards a disease of interest. In some embodiments, a Gene Writing system can be used to insert a protective allele into the genome, e.g., a transgene that expresses a variant of a protein that reduces the risk of developing a particular disease. In some embodiments, integration of a transgene is used to increase the levels of an endogenous protein by providing one or more additional copies. In some embodiments, a Gene Writing system may be used to incorporate a regulatory element, e.g., promoter, enhancer, transcription factor binding site, miRNA binding site, or epigenetic modification site, to alter the expression of an endogenous gene to reduce disease risk or lessen its severity. In some embodiments, a Gene Writing system may be used to replace one or more exons of an endogenous protein to remove an allele that increases disease risk or to alter an allele to one that confers disease protection.

### Plant-Modification Methods

Gene Writer systems described herein may be used to modify a plant or a plant part (e.g., leaves, roots, flowers, fruits, or seeds), e.g., to increase the fitness of a plant.

### A. Delivery to a Plant

Provided herein are methods of delivering a Gene Writer system described herein to a plant. Included are methods for delivering a Gene Writer system to a plant by contacting the plant, or part thereof, with a Gene Writer system. The methods are useful for modifying the plant to, e.g., increase the fitness of a plant.

More specifically, in some embodiments, a nucleic acid described herein (e.g., a nucleic acid encoding a GeneWriter) may be encoded in a vector, e.g., inserted adjacent to a plant promoter, e.g., a maize ubiquitin promoter (ZmUBI) in a plant vector (e.g., pHUC411). In some embodiments, the nucleic acids described herein are introduced into a plant (e.g., japonica rice) or part of a plant (e.g., a callus of a plant) via agrobacteria. In some embodiments, the systems and methods described herein can be used in plants by replacing a plant gene (e.g., hygromycin phosphotransferase (HPT)) with a null allele (e.g., containing a base substitution at the start codon). Systems and methods for modifying a plant genome are described in Xu et. al. Development of plant prime—editing systems for precise genome editing, 2020, Plant Communications.

In one aspect, provided herein is a method of increasing the fitness of a plant, the method including delivering to the plant the Gene Writer system described herein (e.g., in an effective amount and duration) to increase the fitness of the plant relative to an untreated plant (e.g., a plant that has not been delivered the Gene Writer system).

An increase in the fitness of the plant as a consequence of delivery of a Gene Writer system can manifest in a number of ways, e.g., thereby resulting in a better production of the plant, for example, an improved yield, improved vigor of the plant or quality of the harvested product from the plant, an improvement in pre- or post-harvest traits deemed desirable for agriculture or horticulture (e.g., taste, appearance, shelf life), or for an improvement of traits that otherwise benefit humans (e.g., decreased allergen production). An improved yield of a plant relates to an increase in the yield of a product (e.g., as measured by plant biomass, grain, seed or fruit yield, protein content, carbohydrate or oil content or leaf area) of the plant by a measurable amount over the yield of the same product of the plant produced under the same conditions, but without the application of the instant compositions or compared with application of conventional plant-modifying agents. For example, yield can be increased by at least about 0.5%, about 1%, about 2%, about 3%, about 4%, about 5%, about 10%, about 20%, about 30%, about 40%, about 50%, about 60%, about 70%, about 80%, about 90%, about 100%, or more than 100%. In some instances, the method is effective to increase yield by about 2×-fold, 5×-fold, 10×-fold, 25×-fold, 50×-fold, 75×-fold, 100×-fold, or more than 100×-fold relative to an untreated plant. Yield can be expressed in terms of an amount by weight or volume of the plant or a product of the plant on some basis. The basis can be expressed in terms of time, growing area, weight of plants produced, or amount of a raw material used. For example, such methods may increase the yield of plant tissues including, but not limited to: seeds, fruits, kernels, bolls, tubers, roots, and leaves.

An increase in the fitness of a plant as a consequence of delivery of a Gene Writer system can also be measured by other means, such as an increase or improvement of the vigor rating, the stand (the number of plants per unit of area), plant height, stalk circumference, stalk length, leaf number, leaf size, plant canopy, visual appearance (such as greener leaf color), root rating, emergence, protein content, increased tillering, bigger leaves, more leaves, less dead basal leaves, stronger tillers, less fertilizer needed, less seeds needed, more productive tillers, earlier flowering, early grain or seed maturity, less plant verse (lodging), increased shoot growth, earlier germination, or any combination of these factors, by a measurable or noticeable amount over the same factor of the plant produced under the same conditions, but without the administration of the instant compositions or with application of conventional plant-modifying agents.

Accordingly, provided herein is a method of modifying a plant, the method including delivering to the plant an effective amount of any of the Gene Writer systems provided herein, wherein the method modifies the plant and thereby introduces or increases a beneficial trait in the plant (e.g., by about 1%, 2%, 5%, 10%, 20%, 30%, 40%, 50%, 60%, 70%, 80%, 90%, 100%, or more than 100%) relative to an untreated plant. In particular, the method may increase the fitness of the plant (e.g., by about 1%, 2%, 5%10%, 20%, 30%, 40%, 50%, 60%, 70%, 80%, 90%, 100%, or more than 100%) relative to an untreated plant.

In some instances, the increase in plant fitness is an increase (e.g., by about 1%, 2%, 5%, 10%, 20%, 30%, 40%, 50%, 60%, 70%, 80%, 90%, 100%, or more than 100%) in disease resistance, drought tolerance, heat tolerance, cold tolerance, salt tolerance, metal tolerance, herbicide tolerance, chemical tolerance, water use efficiency, nitrogen utilization, resistance to nitrogen stress, nitrogen fixation, pest resistance, herbivore resistance, pathogen resistance, yield, yield under water-limited conditions, vigor, growth, photosynthetic capability, nutrition, protein content, carbohydrate content, oil content, biomass, shoot length, root length, root architecture, seed weight, or amount of harvestable produce.

In some instances, the increase in fitness is an increase (e.g., by about 1%, 2%, 5%, 10%, 20%, 30%, 40%, 50%, 60%, 70%, 80%, 90%, 100%, or more than 100%) in development, growth, yield, resistance to abiotic stressors, or resistance to biotic stressors. An abiotic stress refers to an environmental stress condition that a plant or a plant part is subjected to that includes, e.g., drought stress, salt stress, heat stress, cold stress, and low nutrient stress. A biotic stress refers to an environmental stress condition that a plant or plant part is subjected to that includes, e.g. nematode stress, insect herbivory stress, fungal pathogen stress, bacterial pathogen stress, or viral pathogen stress. The stress may be temporary, e.g. several hours, several days, several months, or permanent, e.g. for the life of the plant.

In some s 10%, 20%, 30%, 40%, 50%, 60%, 70%, 80%, 90%, 100%, or more than 100%) in quality of products harvested from the plant. For example, the increase in plant fitness may be an improvement in commercially favorable features (e.g., taste or appearance) of a product harvested from the plant. In other instances, the increase in plant fitness is an increase in shelf-life of a product harvested from the plant (e.g., by about 1%, 2%, 5%, 10%, 20%, 30%, 40%, 50%, 60%, 70%, 80%, 90%, 100%, or more than 100%).

Alternatively, the increase in fitness may be an alteration of a trait that is beneficial to human or animal health, such as a reduction in allergen production. For example, the increase in fitness may be a decrease (e.g., by about 1%, 2%, 5%, 10%, 20%, 30%, 40%, 50%, 60%, 70%, 80%, 90%, 100%, or more than 100%) in production of an allergen (e.g., pollen) that stimulates an immune response in an animal (e.g., human).

The modification of the plant (e.g., increase in fitness) may arise from modification of one or more plant parts. For example, the plant can be modified by contacting leaf, seed, pollen, root, fruit, shoot, flower, cells, protoplasts, or tissue (e.g., meristematic tissue) of the plant. As such, in another aspect, provided herein is a method of increasing the fitness of a plant, the method including contacting pollen of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In yet another aspect, provided herein is a method of increasing the fitness of a plant, the method including contacting a seed of the plant with an effective amount of any of the Gene Writer systems disclosed herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method including contacting a protoplast of the plant with an effective amount of any of the Gene Writer systems described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In a further aspect, provided herein is a method of increasing the fitness of a plant, the method including contacting a plant cell of the plant with an effective amount of any of the Gene Writer system described herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting meristematic tissue of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

In another aspect, provided herein is a method of increasing the fitness of a plant, the method including contacting an embryo of the plant with an effective amount of any of the plant-modifying compositions herein, wherein the method increases the fitness of the plant (e.g., by about 1%, 2%, 5%, 10%, 20%, 30%, 40%, 50%, 60%, 70%, 80%, 90%, 100%, or more than 100%) relative to an untreated plant.

### B. Application Methods

A plant described herein can be exposed to any of the Gene Writer system compositions described herein in any suitable manner that permits delivering or administering the composition to the plant. The Gene Writer system may be delivered either alone or in combination with other active (e.g., fertilizing agents) or inactive substances and may be applied by, for example, spraying, injection (e.g., microinjection), through plants, pouring, dipping, in the form of concentrated liquids, gels, solutions, suspensions, sprays, powders, pellets, briquettes, bricks and the like, formulated to deliver an effective concentration of the plant-modifying composition. Amounts and locations for application of the compositions described herein are generally determined by the habitat of the plant, the lifecycle stage at which the plant can be targeted by the plant-modifying composition, the site where the application is to be made, and the physical and functional characteristics of the plant-modifying composition.

In some instances, the composition is sprayed directly onto a plant, e.g., crops, by e.g., backpack spraying, aerial spraying, crop spraying/dusting etc. In instances where the Gene Writer system is delivered to a plant, the plant receiving the Gene Writer system may be at any stage of plant growth. For example, formulated plant-modifying compositions can be applied as a seed-coating or root treatment in early stages of plant growth or as a total plant treatment at later stages of the crop cycle. In some instances, the plant-modifying composition may be applied as a topical agent to a plant.

Further, the Gene Writer system may be applied (e.g., in the soil in which a plant grows, or in the water that is used to water the plant) as a systemic agent that is absorbed and distributed through the tissues of a plant. In some instances, plants or food organisms may be genetically transformed to express the Gene Writer system.

Delayed or continuous release can also be accomplished by coating the Gene Writer system or a composition with the plant-modifying composition(s) with a dissolvable or bioerodable coating layer, such as gelatin, which coating dissolves or erodes in the environment of use, to then make the plant-modifying com Gene Writer system position available, or by dispersing the agent in a dissolvable or erodable matrix. Such continuous release and/or dispensing means devices may be advantageously employed to consistently maintain an effective concentration of one or more of the plant-modifying compositions described herein.

In some instances, the Gene Writer system is delivered to a part of the plant, e.g., a leaf, seed, pollen, root, fruit, shoot, or flower, or a tissue, cell, or protoplast thereof. In some instances, the Gene Writer system is delivered to a cell of the plant. In some instances, the Gene Writer system is delivered to a protoplast of the plant. In some instances, the Gene Writer system is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the Gene Writer system is delivered to a plant embryo.

### C. Plants

A variety of plants can be delivered to or treated with a Gene Writer system described herein. Plants that can be delivered a Gene Writer system (i.e., “treated”) in accordance with the present methods include whole plants and parts thereof, including, but not limited to, shoot vegetative organs/structures (e.g., leaves, stems and tubers), roots, flowers and floral organs/structures (e.g., bracts, sepals, petals, stamens, carpels, anthers and ovules), seed (including embryo, endosperm, cotyledons, and seed coat) and fruit (the mature ovary), plant tissue (e.g., vascular tissue, ground tissue, and the like) and cells (e.g., guard cells, egg cells, and the like), and progeny of same. Plant parts can further refer parts of the plant such as the shoot, root, stem, seeds, stipules, leaves, petals, flowers, ovules, bracts, branches, petioles, internodes, bark, pubescence, tillers, rhizomes, fronds, blades, pollen, stamen, and the like.

The class of plants that can be treated in a method disclosed herein includes the class of higher and lower plants, including angiosperms (monocotyledonous and dicotyledonous plants), gymnosperms, ferns, horsetails, psilophytes, lycophytes, bryophytes, and algae (e.g., multicellular or unicellular algae). Plants that can be treated in accordance with the present methods further include any vascular plant, for example monocotyledons or dicotyledons or gymnosperms, including, but not limited to alfalfa, apple, Arabidopsis, banana, barley, canola, castor bean, chrysanthemum, clover, cocoa, coffee, cotton, cottonseed, corn, crambe, cranberry, cucumber, dendrobium, dioscorea, eucalyptus, fescue, flax, gladiolus, liliacea, linseed, millet, muskmelon, mustard, oat, oil palm, oilseed rape, papaya, peanut, pineapple, ornamental plants, Phaseolus, potato, rapeseed, rice, rye, ryegrass, safflower, sesame, sorghum, soybean, sugarbeet, sugarcane, sunflower, strawberry, tobacco, tomato, turfgrass, wheat and vegetable crops such as lettuce, celery, broccoli, cauliflower, cucurbits; fruit and nut trees, such as apple, pear, peach, orange, grapefruit, lemon, lime, almond, pecan, walnut, hazel; vines, such as grapes (e.g., a vineyard), kiwi, hops; fruit shrubs and brambles, such as raspberry, blackberry, gooseberry; forest trees, such as ash, pine, fir, maple, oak, chestnut, popular; with alfalfa, canola, castor bean, corn, cotton, crambe, flax, linseed, mustard, oil palm, oilseed rape, peanut, potato, rice, safflower, sesame, soybean, sugarbeet, sunflower, tobacco, tomato, and wheat. Plants that can be treated in accordance with the methods of the present invention include any crop plant, for example, forage crop, oilseed crop, grain crop, fruit crop, vegetable crop, fiber crop, spice crop, nut crop, turf crop, sugar crop, beverage crop, and forest crop. In certain instances, the crop plant that is treated in the method is a soybean plant. In other certain instances, the crop plant is wheat. In certain instances, the crop plant is corn. In certain instances, the crop plant is cotton. In certain instances, the crop plant is alfalfa. In certain instances, the crop plant is sugarbeet. In certain instances, the crop plant is rice. In certain instances, the crop plant is potato. In certain instances, the crop plant is tomato.

In certain instances, the plant is a crop. Examples of such crop plants include, but are not limited to, monocotyledonous and dicotyledonous plants including, but not limited to, fodder or forage legumes, ornamental plants, food crops, trees, or shrubs selected from Acer spp., Allium spp., Amaranthus spp., Ananas comosus, Apium graveolens, Arachis spp, Asparagus officinalis, Beta vulgaris, Brassica spp. (e.g., Brassica napus, Brassica rapa ssp. (canola, oilseed rape, turnip rape), Camellia sinensis, Canna indica, Cannabis saliva, Capsicum spp., Castanea spp., Cichorium endivia, Citrullus lanatus, Citrus spp., Cocos spp., Coffea spp., Coriandrum sativum, Corylus spp., Crataegus spp., Cucurbita spp., Cucumis spp., Daucus carota, Fagus spp., Ficus carica, Fragaria spp., Ginkgo biloba, Glycine spp. (e.g., Glycine max, Soja hispida or Soja max), Gossypium hirsutum, Helianthus spp. (e.g., Helianthus annuus), Hibiscus spp., Hordeum spp. (e.g., Hordeum vulgare), Ipomoea batatas, Juglans spp., Lactuca sativa, Linum usitatissimum, Litchi chinensis, Lotus spp., Luffa acutangula, Lupinus spp., Lycopersicon spp. (e.g., Lycopersicon esculenturn, Lycopersicon lycopersicum, Lycopersicon pyriforme), Malus spp., Medicago sativa, Mentha spp., Miscanthus sinensis, Morus nigra, Musa spp., Nicotiana spp., Olea spp., Oryza spp. (e.g., Oryza sativa, Oryza latifolia), Panicum miliaceum, Panicum virgatum, Passiflora edulis, Petroselinum crispum, Phaseolus spp., Pinus spp., Pistacia vera, Pisum spp., Poa spp., Populus spp., Prunus spp., Pyrus communis, Quercus spp., Raphanus sativus, Rheum rhabarbarum, Ribes spp., Ricinus communis, Rubus spp., Saccharum spp., Salix sp., Sambucus spp., Secale cereale, Sesamum spp., Sinapis spp., Solanum spp. (e.g., Solanum tuberosum, Solanum integrifolium or Solanum lycopersicum), Sorghum bicolor, Sorghum halepense, Spinacia spp., Tamarindus indica, Theobroma cacao, Trifolium spp., Triticosecale rimpaui, Triticum spp. (e.g., Triticum aestivum, Triticum durum, Triticum turgidum, Triticum hybernum, Triticum macha, Triticum sativum or Triticum vulgare), Vaccinium spp., Vicia spp., Vigna spp., Viola odorata, Vitis spp., and Zea mays. In certain embodiments, the crop plant is rice, oilseed rape, canola, soybean, corn (maize), cotton, sugarcane, alfalfa, sorghum, or wheat.

The plant or plant part for use in the present invention include plants of any stage of plant development. In certain instances, the delivery can occur during the stages of germination, seedling growth, vegetative growth, and reproductive growth. In certain instances, delivery to the plant occurs during vegetative and reproductive growth stages. In some instances, the composition is delivered to pollen of the plant. In some instances, the composition is delivered to a seed of the plant. In some instances, the composition is delivered to a protoplast of the plant. In some instances, the composition is delivered to a tissue of the plant. For example, the composition may be delivered to meristematic tissue of the plant (e.g., apical meristem, lateral meristem, or intercalary meristem). In some instances, the composition is delivered to permanent tissue of the plant (e.g., simple tissues (e.g., parenchyma, collenchyma, or sclerenchyma) or complex permanent tissue (e.g., xylem or phloem)). In some instances, the composition is delivered to a plant embryo. In some instances, the composition is delivered to a plant cell. The stages of vegetative and reproductive growth are also referred to herein as “adult” or “mature” plants.

In instances where the Gene Writer system is delivered to a plant part, the plant part may be modified by the plant-modifying agent. Alternatively, the Gene Writer system may be distributed to other parts of the plant (e.g., by the plant's circulatory system) that are subsequently modified by the plant-modifying agent.

### Lipid Nanoparticles

The methods and systems provided by the invention, may employ any suitable carrier or delivery modality, including, in certain embodiments, lipid nanoparticles (LNPs). Lipid nanoparticles, in some embodiments, comprise one or more ionic lipids, such as non-cationic lipids (e.g., neutral or anionic, or zwitterionic lipids); one or more conjugated lipids (such as PEG-conjugated lipids or lipids conjugated to polymers described in Table 5 of WO2019217941; incorporated herein by reference in its entirety); one or more sterols (e.g., cholesterol); and, optionally, one or more targeting molecules (e.g., conjugated receptors, receptor ligands, antibodies); or combinations of the foregoing.

Lipids that can be used in nanoparticle formations (e.g., lipid nanoparticles) include, for example those described in Table 4 of WO2019217941, which is incorporated by reference e.g., a lipid-containing nanoparticle can comprise one or more of the lipids in Table 4 of WO2019217941. Lipid nanoparticles can include additional elements, such as polymers, such as the polymers described in Table 5 of WO2019217941, incorporated by reference.

In some embodiments, conjugated lipids, when present, can include one or more of PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-O-(2′,3′-di(tetradecanoyloxy)propyl-1-O-(w-methoxy(polyethoxy)ethyl) butanedioate (PEG-S-DMG)), PEG dialkoxypropylcarbam, N-(carbonyl-methoxypoly ethylene glycol 2000)-1,2-distearoyl-sn-glycero-3-phosphoethanolamine sodium salt, and those described in Table 2 of WO2019051289 (incorporated by reference), and combinations of the foregoing.

In some embodiments, sterols that can be incorporated into lipid nanoparticles include one or more of cholesterol or cholesterol derivatives, such as those in WO2009/127060 or US2010/0130588, which are incorporated by reference. Additional exemplary sterols include phytosterols, including those described in Eygeris et al (2020), dx.doi.org/10.1021/acs.nanolett.0c01386, incorporated herein by reference.

In some embodiments, the lipid particle comprises an ionizable lipid, a non-cationic lipid, a conjugated lipid that inhibits aggregation of particles, and a sterol. The amounts of these components can be varied independently and to achieve desired properties. For example, in some embodiments, the lipid nanoparticle comprises an ionizable lipid is in an amount from about 20 mol % to about 90 mol % of the total lipids (in other embodiments it may be 20-70% (mol), 30-60% (mol) or 40-50% (mol); about 50 mol % to about 90 mol % of the total lipid present in the lipid nanoparticle), a non-cationic lipid in an amount from about 5 mol % to about 30 mol % of the total lipids, a conjugated lipid in an amount from about 0.5 mol % to about 20 mol % of the total lipids, and a sterol in an amount from about 20 mol % to about 50 mol % of the total lipids. The ratio of total lipid to nucleic acid (e.g., encoding the Gene Writer or template nucleic acid) can be varied as desired. For example, the total lipid to nucleic acid (mass or weight) ratio can be from about 10:1 to about 30:1.

In some embodiments, the lipid to nucleic acid ratio (mass/mass ratio; w/w ratio) can be in the range of from about 1:1 to about 25:1, from about 10:1 to about 14:1, from about 3:1 to about 15:1, from about 4:1 to about 10:1, from about 5:1 to about 9:1, or about 6:1 to about 9:1. The amounts of lipids and nucleic acid can be adjusted to provide a desired N/P ratio, for example, N/P ratio of 3, 4, 5, 6, 7, 8, 9, 10 or higher. Generally, the lipid nanoparticle formulation's overall lipid content can range from about 5 mg/ml to about 30 mg/mL.

Some non-limiting example of lipid compounds that may be used (e.g., in combination with other lipid components) to form lipid nanoparticles for the delivery of compositions described herein, e.g., nucleic acid (e.g., RNA) described herein (e.g., a template nucleic acid or a nucleic acid encoding a GeneWriter) includes,

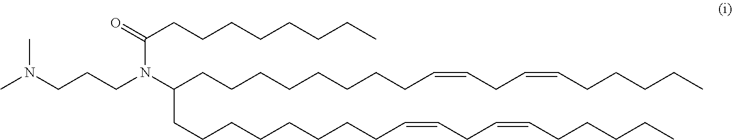 [SMILES: CCCCC/C=C\C/C=C\CCCCCCCCC(CCCCCCCC/C=C\C/C=C\CCCCC)N(CCCN(C)C)C(=O)CCCCCCCC]

In some embodiments an LNP comprising Formula (i) is used to deliver a GeneWriter composition described herein to the liver and/or hepatocyte cells.

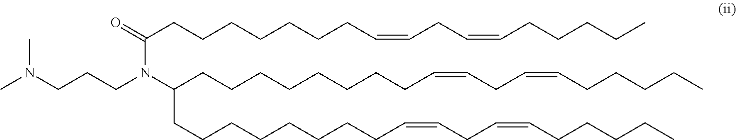 [SMILES: CCCCC/C=C\C/C=C\CCCCCCCCC(CCCCCCCC/C=C\C/C=C\CCCCC)N(CCCN(C)C)C(=O)CCCCCCC/C=C\C/C=C\CCCCC]

In some embodiments an LNP comprising Formula (ii) is used to deliver a GeneWriter composition described herein to the liver and/or hepatocyte cells.

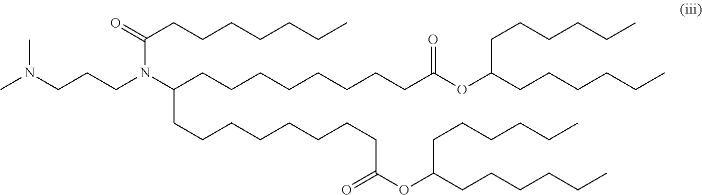 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC]

In some embodiments an LNP comprising Formula (iii) is used to deliver a GeneWriter composition described herein to the liver and/or hepatocyte cells.

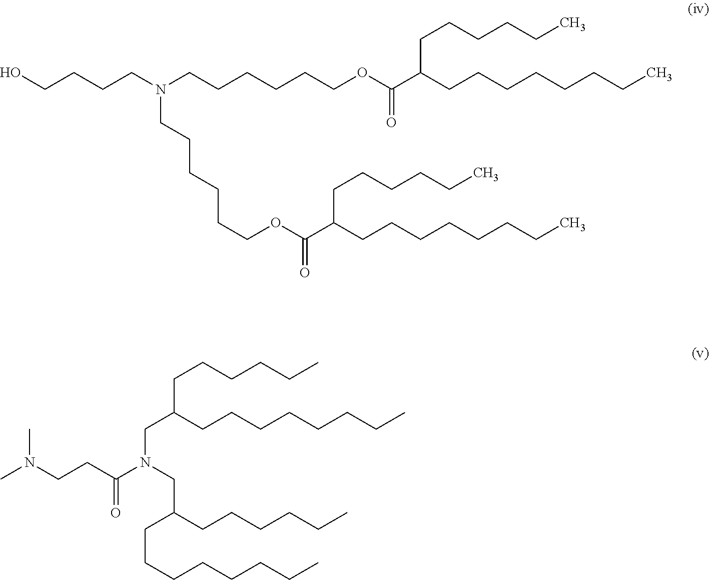

In some embodiments an LNP comprising Formula (v) is used to deliver a GeneWriter composition described herein to the liver and/or hepatocyte cells.

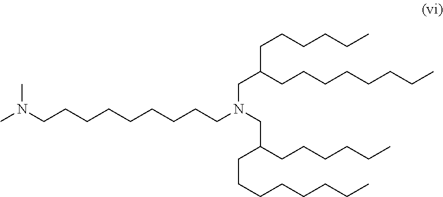 [SMILES: CCCCCCCCC(CCCCCC)CN(CCCCCCCCCN(C)C)CC(CCCCCC)CCCCCCCC]

In some embodiments an LNP comprising Formula (vi) is used to deliver a GeneWriter composition described herein to the liver and/or hepatocyte cells.

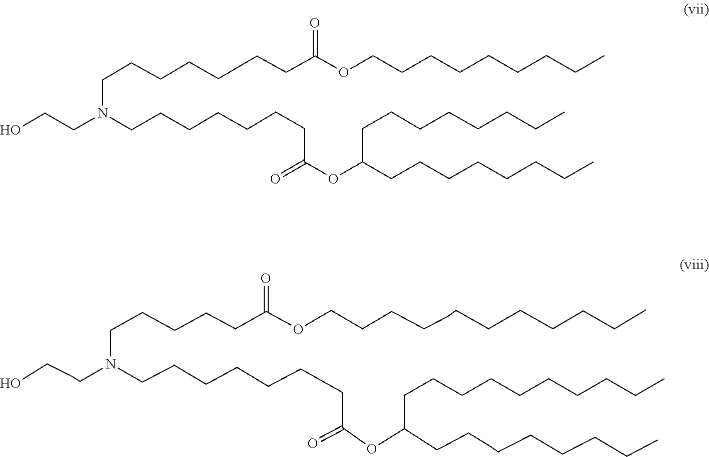 [SMILES: CCCCCCCCCOC(=O)CCCCCCCCN(CCO)CCCCCCCC(=O)OC(CCCCCCCC)CCCCCCCC ; CCCCCCCCCCCCOC(=O)CCCCCN(CCCO)CCCCCCCC(=O)OC(CCCCCCCCC)CCCCCCCCCC]

In some embodiments an LNP comprising Formula (viii) is used to deliver a GeneWriter composition described herein to the liver and/or hepatocyte cells.

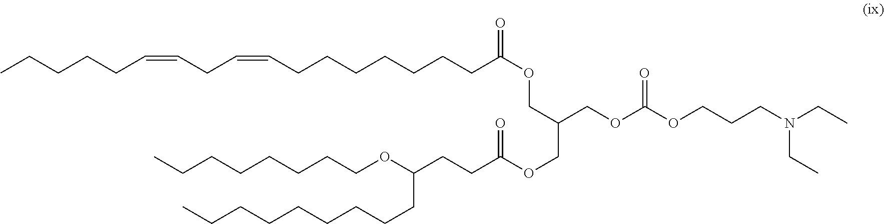 [SMILES: CCCCC/C=C\C/C=C\CCCCCCCC(=O)OCC(COC(=O)CCC(CCCCCCCCC)OCCCCCCCC)COC(=O)OCCCN(CC)CC]

In some embodiments an LNP comprising Formula (ix) is used to deliver a GeneWriter composition described herein to the liver and/or hepatocyte cells.

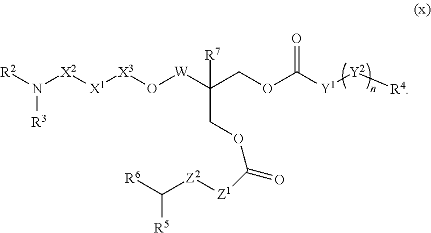

wherein
X¹ is O, NR¹, or a direct bond, X² is C2-5 alkylene, X³ is C(═O) or a direct bond, R¹ is H or Me, R³ is Ci-3 alkyl, R² is Ci-3 alkyl, or R² taken together with the nitrogen atom to which it is attached and 1-3 carbon atoms of X² form a 4-, 5-, or 6-membered ring, or X¹ is NR¹, R¹ and R² taken together with the nitrogen atoms to which they are attached form a 5- or 6-membered ring, or R² taken together with R³ and the nitrogen atom to which they are attached form a 5-, 6-, or 7-membered ring, Y¹ is C2-12 alkylene, Y² is selected from

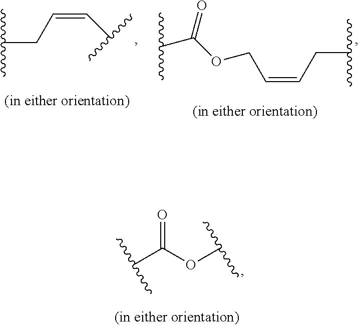 [SMILES: */C=C\C* ; *C/C=C\COC(*)=O ; *OC(*)=O]

n is 0 to 3, R⁴ is Ci-15 alkyl, Z¹ is Ci-6 alkylene or a direct bond,

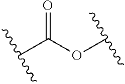 [SMILES: *OC(*)=O]

Z² is
(in either orientation) or absent, provided that if Z¹ is a direct bond, Z² is absent;
R⁵ is C5-9 alkyl or C6-10 alkoxy, R⁶ is C5-9 alkyl or C6-10 alkoxy, W is methylene or a direct bond, and R⁷ is H or Me, or a salt thereof, provided that if R³ and R² are C2 alkyls, X¹ is O, X² is linear C3 alkylene, X³ is C(═O), Y¹ is linear Ce alkylene, (Y²)n-R⁴ is

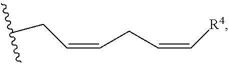

R⁴ is linear C5 alkyl, Z¹ is C2 alkylene, Z² is absent, W is methylene, and R⁷ is H, then R⁵ and R6 are not Cx alkoxy.

In some embodiments an LNP comprising Formula (xii) is used to deliver a GeneWriter composition described herein to the liver and/or hepatocyte cells.

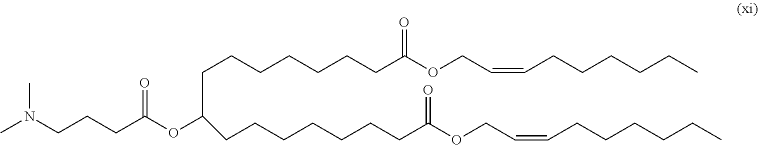 [SMILES: CCCCCC/C=C\COC(=O)CCCCCCCC(CCCCCCCC(=O)OC/C=C\CCCCCC)OC(=O)CCCN(C)C]

In some embodiments an LNP comprising Formula (xi) is used to deliver a GeneWriter composition described herein to the liver and/or hepatocyte cells.

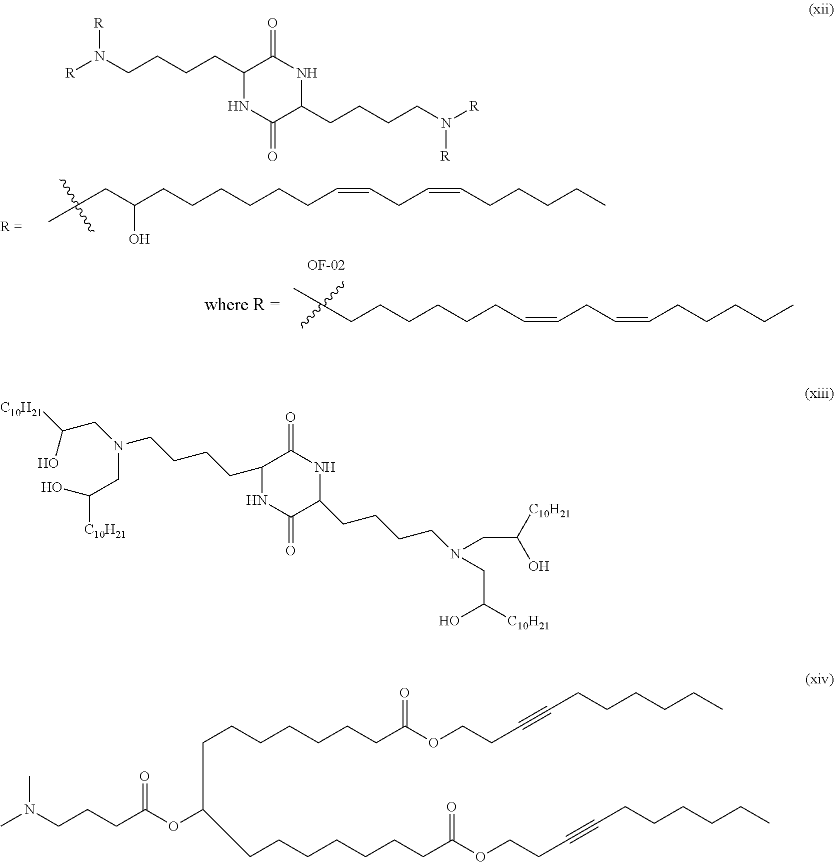

In some embodiments an LNP comprises a compound of Formula (xiii) and a compound of Formula (xiv).

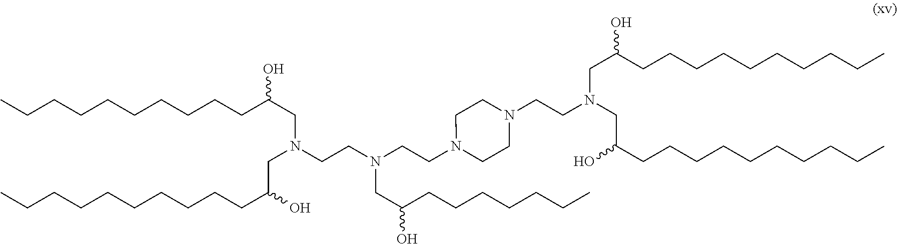

In some embodiments an LNP comprising Formula (xv) is used to deliver a GeneWriter composition described herein to the liver and/or hepatocyte cells.

### PEI600 Core

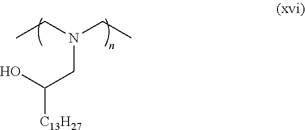

In some embodiments an LNP comprising a formulation of Formula (xvi) is used to deliver a GeneWriter composition described herein to the lung endothelial cells.

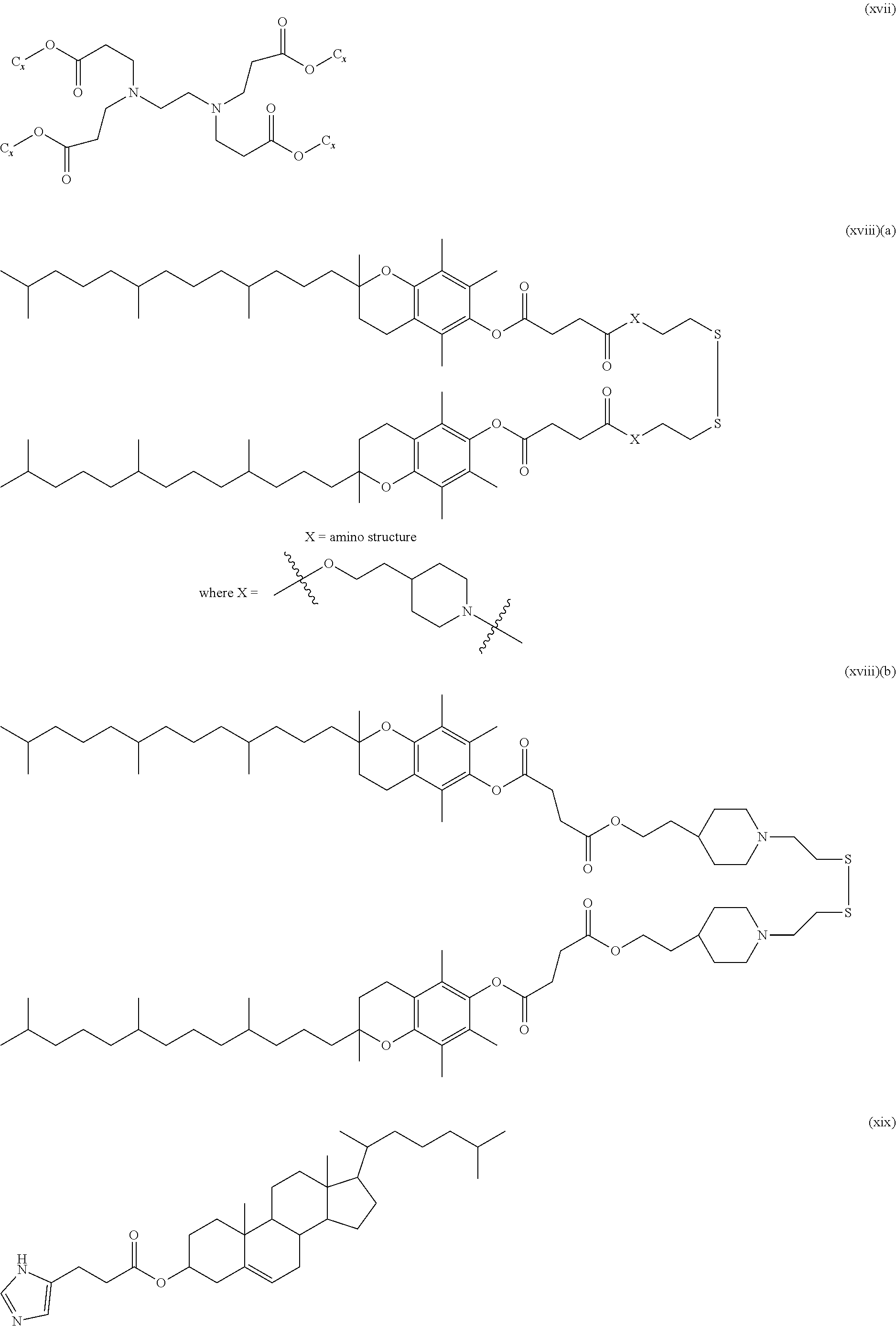

In some embodiments, a lipid compound used to form lipid nanoparticles for the delivery of compositions described herein, e.g., nucleic acid (e.g., RNA) described herein (e.g., a template nucleic acid or a nucleic acid encoding a GeneWriter) is made by one of the following reactions:

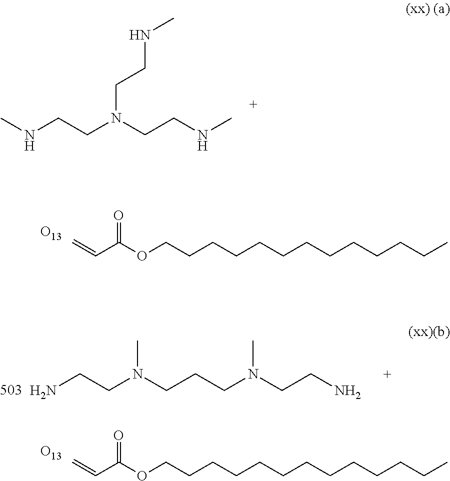

In some embodiments, a composition described herein (e.g., a nucleic acid or a protein) is provided in an LNP that comprises an ionizable lipid. In some embodiments, the ionizable lipid is heptadecan-9-yl 8-((2-hydroxyethyl)(6-oxo-6-(undecyloxy)hexyl)amino)octanoate (SM-102); e.g., as described in Example 1 of US9,867,888 (incorporated by reference here in its entirety). In some embodiments, the ionizable lipid is 9Z,12Z)-3-((4,4-bis(octyloxy)butanoyl)oxy)-2-((((3-(diethylamino)propoxy)carbonyl)oxy)methyl)propyl octadeca-9,12-dienoate (LPO1), e.g., as synthesized in Example 13 of WO2015/095340 (incorporated by reference herein in its entirety). In some embodiments, the ionizable lipid is Di((Z)-non-2-en-1-yl) 9-((4-dimethylamino)butanoyl)oxy)heptadecanedioate (L319), e.g. as synthesized in Example 7, 8, or 9 of US2012/0027803 (incorporated by reference herein in its entirety). In some embodiments, the ionizable lipid is 1,1′-((2-(4-(2-((2-(Bis(2-hydroxydodecyl)amino)ethyl)(2-hydroxydodecyl) amino)ethyl)piperazin-1-yl)ethyl)azanediyl)bis(dodecan-2-ol) (C12-200), e.g., as synthesized in Examples 14 and 16 of WO2010/053572 (incorporated by reference herein in its entirety). In some embodiments, the ionizable lipid is Imidazole cholesterol ester (ICE) lipid (3S, 10R, 13R, 17R)-10, 13-dimethyl-17-((R)-6-methylheptan-2-yl)-2, 3, 4, 7, 8, 9, 10, 11, 12, 13, 14, 15, 16, 17-tetradecahydro-lH-cyclopenta[a]phenanthren-3-yl 3-(1H-imidazol-4-yl)propanoate, e.g., Structure (I) from WO2020/106946 (incorporated by reference herein in its entirety).

In some embodiments, an ionizable lipid may be a cationic lipid, an ionizable cationic lipid, e.g., a cationic lipid that can exist in a positively charged or neutral form depending on pH, or an amine-containing lipid that can be readily protonated. In some embodiments, the cationic lipid is a lipid capable of being positively charged, e.g., under physiological conditions. Exemplary cationic lipids include one or more amine group(s) which bear the positive charge. In some embodiments, the lipid particle comprises a cationic lipid in formulation with one or more of neutral lipids, ionizable amine-containing lipids, biodegradable alkyn lipids, steroids, phospholipids including polyunsaturated lipids, structural lipids (e.g., sterols), PEG, cholesterol and polymer conjugated lipids. In some embodiments, the cationic lipid may be an ionizable cationic lipid. An exemplary cationic lipid as disclosed herein may have an effective pKa over 6.0. In embodiments, a lipid nanoparticle may comprise a second cationic lipid having a different effective pKa (e.g., greater than the first effective pKa), than the first cationic lipid. A lipid nanoparticle may comprise between 40 and 60 mol percent of a cationic lipid, a neutral lipid, a steroid, a polymer conjugated lipid, and a therapeutic agent, e.g., a nucleic acid (e.g., RNA) described herein (e.g., a template nucleic acid or a nucleic acid encoding a GeneWriter), encapsulated within or associated with the lipid nanoparticle. In some embodiments, the nucleic acid is co-formulated with the cationic lipid. The nucleic acid may be adsorbed to the surface of an LNP, e.g., an LNP comprising a cationic lipid. In some embodiments, the nucleic acid may be encapsulated in an LNP, e.g., an LNP comprising a cationic lipid. In some embodiments, the lipid nanoparticle may comprise a targeting moiety, e.g., coated with a targeting agent. In embodiments, the LNP formulation is biodegradable. In some embodiments, a lipid nanoparticle comprising one or more lipid described herein, e.g., Formula (i), (ii), (ii), (vii) and/or (ix) encapsulates at least 1%, at least 5%, at least 10%, at least 20%, at least 30%, at least 40%, at least 50%, at least 60%, at least 70%, at least 80%, at least 90%, at least 92%, at least 95%, at least 97%, at least 98% or 100% of an RNA molecule, e.g., template RNA and/or a mRNA encoding the Gene Writer polypeptide.

Exemplary ionizable lipids that can be used in lipid nanoparticle formulations include, without limitation, those listed in Table 1 of WO2019051289, incorporated herein by reference. Additional exemplary lipids include, without limitation, one or more of the following formulae: X of US2016/0311759; I of US20150376115 or in US2016/0376224; I, II or III of US20160151284; I, IA, II, or IIA of US20170210967; I-c of US20150140070; A of US2013/0178541; I of US2013/0303587 or US2013/0123338; I of US2015/0141678; II, III, IV, or V of US2015/0239926; I of US2017/0119904; I or II of WO2017/117528; A of US2012/0149894; A of US2015/0057373; A of WO2013/116126; A of US2013/0090372; A of US2013/0274523; A of US2013/0274504; A of US2013/0053572; A of WO2013/016058; A of WO2012/162210; I of US2008/042973; I, II, III, or IV of US2012/01287670; I or II of US2014/0200257; I, II, or III of US2015/0203446; I or III of US2015/0005363; I, IA, IB, IC, ID, II, IIA, IIB, IIC, IID, or III-XXIV of US2014/0308304; of US2013/0338210; I, II, III, or IV of WO2009/132131; A of US2012/01011478; I or XXXV of US2012/0027796; XIV or XVII of US2012/0058144; of US2013/0323269; I of US2011/0117125; I, II, or III of US2011/0256175; I, II, III, IV, V, VI, VII, VIII, IX, X, XI, XII of US2012/0202871; I, II, III, IV, V, VI, VII, VIII, X, XII, XIII, XIV, XV, or XVI of US2011/0076335; I or II of US2006/008378; I of US2013/0123338; I or X-A-Y-Z of US2015/0064242; XVI, XVII, or XVIII of US2013/0022649; I, II, or III of US2013/0116307; I, II, or III of US2013/0116307; I or II of US2010/0062967; I-X of US2013/0189351; I of US2014/0039032; V of US2018/0028664; I of US2016/0317458; I of US2013/0195920; 5, 6, or 10 of U.S. Pat. No. 10,221,127; 111-3 of WO2018/081480; I-5 or 1-8 of WO2020/081938; 18 or 25 of U.S. Pat. No. 9,867,888; A of US2019/0136231; II of WO2020/219876; 1 of US2012/0027803; OF-02 of US2019/0240349; 23 of U.S. Pat. No. 10,086,013; cKK-E12/A6 of Miao et al (2020); C12-200 of WO2010/053572; 7C1 of Dahlman et al (2017); 304-013 or 503-013 of Whitehead et al; TS-P4C2 of U.S. Pat. No. 9,708,628; I of WO2020/106946; I of WO2020/106946.

In some embodiments, the ionizable lipid is MC3 (6Z,9Z,28Z,3 lZ)-heptatriaconta-6,9,28,3 1-tetraen-19-yl-4-(dimethylamino) butanoate (DLin-MC3-DMA or MC3), e.g., as described in Example 9 of WO2019051289A9 (incorporated by reference herein in its entirety). In some embodiments, the ionizable lipid is the lipid ATX-002, e.g., as described in Example 10 of WO2019051289A9 (incorporated by reference herein in its entirety). In some embodiments, the ionizable lipid is (13Z,16Z)-A,A-dimethyl-3-nonyldocosa-13, 16-dien-1-amine (Compound 32), e.g., as described in Example 11 of WO2019051289A9 (incorporated by reference herein in its entirety). In some embodiments, the ionizable lipid is Compound 6 or Compound 22, e.g., as described in Example 12 of WO2019051289A9 (incorporated by reference herein in its entirety).

Exemplary non-cationic lipids include, but are not limited to, distearoyl-sn-glycero-phosphoethanolamine, distearoylphosphatidylcholine (DSPC), dioleoylphosphatidylcholine (DOPC), dipalmitoylphosphatidylcholine (DPPC), dioleoylphosphatidylglycerol (DOPG), dipalmitoylphosphatidylglycerol (DPPG), dioleoyl-phosphatidylethanolamine (DOPE), palmitoyloleoylphosphatidylcholine (POPC), palmitoyloleoylphosphatidylethanolamine (POPE), dioleoyl-phosphatidylethanolamine 4-(N-maleimidomethyl)-cyclohexane-1-carboxylate (DOPE-mal), dipalmitoyl phosphatidyl ethanolamine (DPPE), dimyristoylphosphoethanolamine (DMPE), distearoyl-phosphatidyl-ethanolamine (DSPE), monomethyl-phosphatidylethanolamine (such as 16-O-monomethyl PE), dimethyl-phosphatidylethanolamine (such as 16-O-dimethyl PE), 18-1-trans PE, 1-stearoyl-2-oleoyl-phosphatidyethanolamine (SOPE), hydrogenated soy phosphatidylcholine (HSPC), egg phosphatidylcholine (EPC), dioleoylphosphatidylserine (DOPS), sphingomyelin (SM), dimyristoyl phosphatidylcholine (DMPC), dimyristoyl phosphatidylglycerol (DMPG), distearoylphosphatidylglycerol (DSPG), dierucoylphosphatidylcholine (DEPC), palmitoyloleyolphosphatidylglycerol (POPG), dielaidoyl-phosphatidylethanolamine (DEPE), lecithin, phosphatidylethanolamine, lysolecithin, lysophosphatidylethanolamine, phosphatidylserine, phosphatidylinositol, sphingomyelin, egg sphingomyelin (ESM), cephalin, cardiolipin, phosphatidicacid,cerebrosides, dicetylphosphate, lysophosphatidylcholine, dilinoleoylphosphatidylcholine, or mixtures thereof. It is understood that other diacylphosphatidylcholine and diacylphosphatidylethanolamine phospholipids can also be used. The acyl groups in these lipids are preferably acyl groups derived from fatty acids having C10-C24 carbon chains, e.g., lauroyl, myristoyl, paimitoyl, stearoyl, or oleoyl. Additional exemplary lipids, in certain embodiments, include, without limitation, those described in Kim et al. (2020) dx.doi.org/10.1021/acs.nanolett.0c01386, incorporated herein by reference. Such lipids include, in some embodiments, plant lipids found to improve liver transfection with mRNA (e.g., DGTS).

Other examples of non-cationic lipids suitable for use in the lipid nanoparticles include, without limitation, nonphosphorous lipids such as, e.g., stearylamine, dodeeylamine, hexadecylamine, acetyl palmitate, glycerol ricinoleate, hexadecyl stereate, isopropyl myristate, amphoteric acrylic polymers, triethanolamine-lauryl sulfate, alkyl-aryl sulfate polyethyloxylated fatty acid amides, dioctadecyl dimethyl ammonium bromide, ceramide, sphingomyelin, and the like. Other non-cationic lipids are described in WO2017/099823 or US patent publication US2018/0028664, the contents of which is incorporated herein by reference in their entirety.

In some embodiments, the non-cationic lipid is oleic acid or a compound of Formula I, II, or IV of US2018/0028664, incorporated herein by reference in its entirety. The non-cationic lipid can comprise, for example, 0-30% (mol) of the total lipid present in the lipid nanoparticle.

In some embodiments, the non-cationic lipid content is 5-20% (mol) or 10-15% (mol) of the total lipid present in the lipid nanoparticle. In embodiments, the molar ratio of ionizable lipid to the neutral lipid ranges from about 2:1 to about 8:1 (e.g., about 2:1, 3:1, 4:1, 5:1, 6:1, 7:1, or 8:1).

In some embodiments, the lipid nanoparticles do not comprise any phospholipids.

In some aspects, the lipid nanoparticle can further comprise a component, such as a sterol, to provide membrane integrity. One exemplary sterol that can be used in the lipid nanoparticle is cholesterol and derivatives thereof. Non-limiting examples of cholesterol derivatives include polar analogues such as 5a-choiestanol, 53-coprostanol, cholesteryl-(2′-hydroxy)-ethyl ether, cholesteryls-(4′-hydroxy)-butyl ether, and 6-ketocholestanol; non-polar analogues such as 5a-cholestane, cholestenone, 5a-cholestanone, 5p-cholestanone, and cholesteryl decanoate; and mixtures thereof. In some embodiments, the cholesterol derivative is a polar analogue, e.g., cholesteryls-(4′-hydroxy)-butyl ether. Exemplary cholesterol derivatives are described in PCT publication WO2009/127060 and US patent publication US2010/0130588, each of which is incorporated herein by reference in its entirety.

In some embodiments, the component providing membrane integrity, such as a sterol, can comprise 0-50% (mol) (e.g., 0-10%, 10-20%, 20-30%, 30-40%, or 40-50%) of the total lipid present in the lipid nanoparticle. In some embodiments, such a component is 20-50% (mol) 30-40% (mol) of the total lipid content of the lipid nanoparticle.

In some embodiments, the lipid nanoparticle can comprise a polyethylene glycol (PEG) or a conjugated lipid molecule. Generally, these are used to inhibit aggregation of lipid nanoparticles and/or provide steric stabilization. Exemplary conjugated lipids include, but are not limited to, PEG-lipid conjugates, polyoxazoline (POZ)-lipid conjugates, polyamide-lipid conjugates (such as ATTA-lipid conjugates), cationic-polymer lipid (CPL) conjugates, and mixtures thereof. In some embodiments, the conjugated lipid molecule is a PEG-lipid conjugate, for example, a (methoxy polyethylene glycol)-conjugated lipid.

Exemplary PEG-lipid conjugates include, but are not limited to, PEG-diacylglycerol (DAG) (such as 1-(monomethoxy-polyethyleneglycol)-2,3-dimyristoylglycerol (PEG-DMG)), PEG-dialkyloxypropyl (DAA), PEG-phospholipid, PEG-ceramide (Cer), a pegylated phosphatidylethanoloamine (PEG-PE), PEG succinate diacylglycerol (PEGS-DAG) (such as 4-0-(2′,3′-di(tetradecanoyloxy)propyl-l-0-(w-methoxy(polyethoxy)ethyl) butanedioate (PEG-S-DMG)), PEG dialkoxypropylcarbam, N-(carbonyl-methoxypolyethylene glycol 2000)-1,2-distearoyl-sn-glycero-3-phosphoethanolamine sodium salt, or a mixture thereof. Additional exemplary PEG-lipid conjugates are described, for example, in U.S. Pat. Nos. 5,885,613, 6,287,591, US2003/0077829, US2003/0077829, US2005/0175682, US2008/0020058, US2011/0117125, US2010/0130588, US2016/0376224, US2017/0119904, and US/099823, the contents of all of which are incorporated herein by reference in their entirety. In some embodiments, a PEG-lipid is a compound of Formula III, III-a-I, III-a-2, III-b-1, III-b-2, or V of US2018/0028664, the content of which is incorporated herein by reference in its entirety. In some embodiments, a PEG-lipid is of Formula II of US20150376115 or US2016/0376224, the content of both of which is incorporated herein by reference in its entirety. In some embodiments, the PEG-DAA conjugate can be, for example, PEG-dilauryloxypropyl, PEG-dimyristyloxypropyl, PEG-dipalmityloxypropyl, or PEG-distearyloxypropyl. The PEG-lipid can be one or more of PEG-DMG, PEG-dilaurylglycerol, PEG-dipalmitoylglycerol, PEG-disterylglycerol, PEG-dilaurylglycamide, PEG-dimyristylglycamide, PEG-dipalmitoylglycamide, PEG-disterylglycamide, PEG-cholesterol (1-[8′-(Cholest-5-en-3[beta]-oxy)carboxamido-3′,6′-dioxaoctanyl]carbamoyl-[omega]-methyl-poly(ethylene glycol), PEG-DMB (3,4-Ditetradecoxylbenzyl-[omega]-methyl-poly(ethylene glycol) ether), and 1,2-dimyristoyl-sn-glycero-3-phosphoethanolamine-N-[methoxy(polyethylene glycol)-2000]. In some embodiments, the PEG-lipid comprises PEG-DMG, 1,2-dimyristoyl-sn-glycero-3-phosphoethanolamine-N-[methoxy(polyethylene glycol)-2000]. In some embodiments, the PEG-lipid comprises a structure selected from:

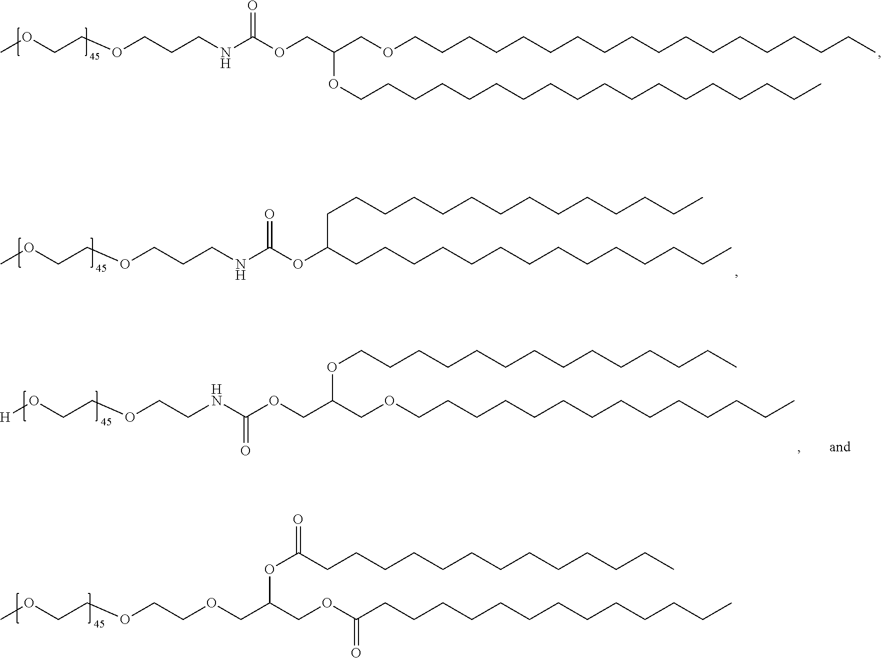

In some embodiments, lipids conjugated with a molecule other than a PEG can also bel used in place of PEG-lipid. For example, polyoxazoline (POZ)-lipid conjugates, polyamide-lipid conjugates (such as ATTA-lipid conjugates), and cationic-polymer lipid (GPL) conjugates can be used in place of or in addition to the PEG-lipid.

Exemplary conjugated lipids, i.e., PEG-lipids, (POZ)-lipid conjugates, ATTA-lipid conjugates and cationic polymer-lipids are described in the PCT and LIS patent applications listed in Table 2 of WO2019051289A9, the contents of all of which are incorporated herein by reference in their entirety.

In some embodiments, the PEG or the conjugated lipid can comprise 0-20% (mol) of the total lipid present in the lipid nanoparticle. In some embodiments, PEG or the conjugated lipid content is 0.5-10% or 2-5% (mol) of the total lipid present in the lipid nanoparticle. Molar ratios of the ionizable lipid, non-cationic-lipid, sterol, and PEG/conjugated lipid can be varied as needed. For example, the lipid particle can comprise 30-70% ionizable lipid by mole or by total weight of the composition, 0-60% cholesterol by mole or by total weight of the composition, 0-30% non-cationic-lipid by mole or by total weight of the composition and 1-10% conjugated lipid by mole or by total weight of the composition. Preferably, the composition comprises 30-40% ionizable lipid by mole or by total weight of the composition, 40-50% cholesterol by mole or by total weight of the composition, and 10-20% non-cationic-lipid by mole or by total weight of the composition. In some other embodiments, the composition is 50-75% ionizable lipid by mole or by total weight of the composition, 20-40% cholesterol by mole or by total weight of the composition, and 5 to 10% non-cationic-lipid, by mole or by total weight of the composition and 1-10% conjugated lipid by mole or by total weight of the composition. The composition may contain 60-70% ionizable lipid by mole or by total weight of the composition, 25-35% cholesterol by mole or by total weight of the composition, and 5-10% non-cationic-lipid by mole or by total weight of the composition. The composition may also contain up to 90% ionizable lipid by mole or by total weight of the composition and 2 to 15% non-cationic lipid by mole or by total weight of the composition. The formulation may also be a lipid nanoparticle formulation, for example comprising 8-30% ionizable lipid by mole or by total weight of the composition, 5-30% non-cationic lipid by mole or by total weight of the composition, and 0-20% cholesterol by mole or by total weight of the composition; 4-25% ionizable lipid by mole or by total weight of the composition, 4-25% non-cationic lipid by mole or by total weight of the composition, 2 to 25% cholesterol by mole or by total weight of the composition, 10 to 35% conjugate lipid by mole or by total weight of the composition, and 5% cholesterol by mole or by total weight of the composition; or 2-30% ionizable lipid by mole or by total weight of the composition, 2-30% non-cationic lipid by mole or by total weight of the composition, 1 to 15% cholesterol by mole or by total weight of the composition, 2 to 35% conjugate lipid by mole or by total weight of the composition, and 1-20% cholesterol by mole or by total weight of the composition; or even up to 90% ionizable lipid by mole or by total weight of the composition and 2-10% non-cationic lipids by mole or by total weight of the composition, or even 100% cationic lipid by mole or by total weight of the composition. In some embodiments, the lipid particle formulation comprises ionizable lipid, phospholipid, cholesterol and a PEG-ylated lipid in a molar ratio of 50:10:38.5:1.5. In some other embodiments, the lipid particle formulation comprises ionizable lipid, cholesterol and a PEG-ylated lipid in a molar ratio of 60:38.5:1.5.

In some embodiments, the lipid particle comprises ionizable lipid, non-cationic lipid (e.g. phospholipid), a sterol (e.g., cholesterol) and a PEG-ylated lipid, where the molar ratio of lipids ranges from 20 to 70 mole percent for the ionizable lipid, with a target of 40-60, the mole percent of non-cationic lipid ranges from 0 to 30, with a target of 0 to 15, the mole percent of sterol ranges from 20 to 70, with a target of 30 to 50, and the mole percent of PEG-ylated lipid ranges from 1 to 6, with a target of 2 to 5.

In some embodiments, the lipid particle comprises ionizable lipid/non-cationic-lipid/sterol/conjugated lipid at a molar ratio of 50:10:38.5:1.5.

In an aspect, the disclosure provides a lipid nanoparticle formulation comprising phospholipids, lecithin, phosphatidylcholine and phosphatidylethanolamine.

In some embodiments, one or more additional compounds can also be included. Those compounds can be administered separately or the additional compounds can be included in the lipid nanoparticles of the invention. In other words, the lipid nanoparticles can contain other compounds in addition to the nucleic acid or at least a second nucleic acid, different than the first. Without limitations, other additional compounds can be selected from the group consisting of small or large organic or inorganic molecules, monosaccharides, disaccharides, trisaccharides, oligosaccharides, polysaccharides, peptides, proteins, peptide analogs and derivatives thereof, peptidomimetics, nucleic acids, nucleic acid analogs and derivatives, an extract made from biological materials, or any combinations thereof.

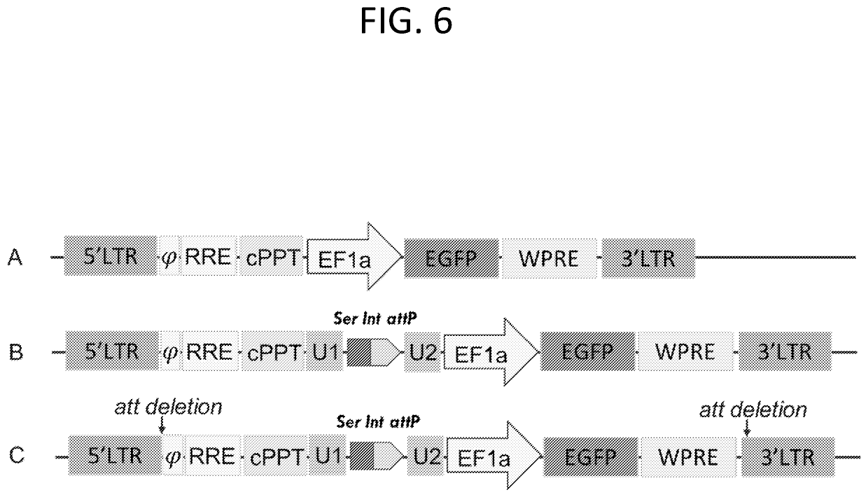

In some embodiments, LNPs are directed to specific tissues by the addition of targeting domains. For example, biological ligands may be displayed on the surface of LNPs to enhance interaction with cells displaying cognate receptors, thus driving association with and cargo delivery to tissues wherein cells express the receptor. In some embodiments, the biological ligand may be a ligand that drives delivery to the liver, e.g., LNPs that display GalNAc result in delivery of nucleic acid cargo to hepatocytes that display asialoglycoprotein receptor (ASGPR). The work of Akinc et al. Mol Ther 18(7):1357-1364 (2010) teaches the conjugation of a trivalent GalNAc ligand to a PEG-lipid (GalNAc-PEG-DSG) to yield LNPs dependent on ASGPR for observable LNP cargo effect (see, e.g., FIG. 6 of Akinc et al. 2010, supra). Other ligand-displaying LNP formulations, e.g., incorporating folate, transferrin, or antibodies, are discussed in WO2017223135, which is incorporated herein by reference in its entirety, in addition to the references used therein, namely Kolhatkar et al., Curr Drug Discov Technol. 2011 8:197-206; Musacchio and Torchilin, Front Biosci. 2011 16:1388-1412; Yu et al., Mol Membr Biol. 2010 27:286-298; Patil et al., Crit Rev Ther Drug Carrier Syst. 2008 25:1-61; Benoit et al., Biomacromolecules. 2011 12:2708-2714; Zhao et al., Expert Opin Drug Deliv. 2008 5:309-319; Akinc et al., Mol Ther. 2010 18:1357-1364; Srinivasan et al., Methods Mol Biol. 2012 820:105-116; Ben-Arie et al., Methods Mol Biol. 2012 757:497-507; Peer 2010 J Control Release. 20:63-68; Peer et al., Proc Natl Acad Sci USA. 2007 104:4095-4100; Kim et al., Methods Mol Biol. 2011 721:339-353; Subramanya et al., Mol Ther. 2010 18:2028-2037; Song et al., Nat Biotechnol. 2005 23:709-717; Peer et al., Science. 2008 319:627-630; and Peer and Lieberman, Gene Ther. 2011 18:1127-1133.

In some embodiments, LNPs are selected for tissue-specific activity by the addition of a Selective ORgan Targeting (SORT) molecule to a formulation comprising traditional components, such as ionizable cationic lipids, amphipathic phospholipids, cholesterol and poly(ethylene glycol) (PEG) lipids. The teachings of Cheng et al. Nat Nanotechnol 15(4):313-320 (2020) demonstrate that the addition of a supplemental “SORT” component precisely alters the in vivo RNA delivery profile and mediates tissue-specific (e.g., lungs, liver, spleen) gene delivery and editing as a function of the percentage and biophysical property of the SORT molecule.

In some embodiments, the LNPs comprise biodegradable, ionizable lipids. In some embodiments, the LNPs comprise (9Z,12Z)-3-((4,4-bis(octyloxy)butanoyl)oxy)-2-((((3-(diethylamino)propoxy)carbonyl)oxy)methyl)propyl octadeca-9,12-dienoate, also called 3-((4,4-bis(octyloxy)butanoyl)oxy)-2-((((3-(diethylamino)propoxy)carbonyl)oxy)methyl)propyl (9Z,12Z)-octadeca-9,12-dienoate) or another ionizable lipid. See, e.g, lipids of WO2019/067992, WO/2017/173054, WO2015/095340, and WO2014/136086, as well as references provided therein. In some embodiments, the term cationic and ionizable in the context of LNP lipids is interchangeable, e.g., wherein ionizable lipids are cationic depending on the pH.

In some embodiments, multiple components of a Gene Writer system may be prepared as a single LNP formulation, e.g., an LNP formulation comprises mRNA encoding for the Gene Writer polypeptide and an RNA template. Ratios of nucleic acid components may be varied in order to maximize the properties of a therapeutic. In some embodiments, the ratio of RNA template to mRNA encoding a Gene Writer polypeptide is about 1:1 to 100:1, e.g., about 1:1 to 20:1, about 20:1 to 40:1, about 40:1 to 60:1, about 60:1 to 80:1, or about 80:1 to 100:1, by molar ratio. In other embodiments, a system of multiple nucleic acids may be prepared by separate formulations, e.g., one LNP formulation comprising a template RNA and a second LNP formulation comprising an mRNA encoding a Gene Writer polypeptide. In some embodiments, the system may comprise more than two nucleic acid components formulated into LNPs. In some embodiments, the system may comprise a protein, e.g., a Gene Writer polypeptide, and a template RNA formulated into at least one LNP formulation.

In some embodiments, the average LNP diameter of the LNP formulation may be between 10s of nm and 100s of nm, e.g., measured by dynamic light scattering (DLS). In some embodiments, the average LNP diameter of the LNP formulation may be from about 40 nm to about 150 nm, such as about 40 nm, 45 nm, 50 nm, 55 nm, 60 nm, 65 nm, 70 nm, 75 nm, 80 nm, 85 nm, 90 nm, 95 nm, 100 nm, 105 nm, 110 nm, 115 nm, 120 nm, 125 nm, 130 nm, 135 nm, 140 nm, 145 nm, or 150 nm. In some embodiments, the average LNP diameter of the LNP formulation may be from about 50 nm to about 100 nm, from about 50 nm to about 90 nm, from about 50 nm to about 80 nm, from about 50 nm to about 70 nm, from about 50 nm to about 60 nm, from about 60 nm to about 100 nm, from about 60 nm to about 90 nm, from about 60 nm to about 80 nm, from about 60 nm to about 70 nm, from about 70 nm to about 100 nm, from about 70 nm to about 90 nm, from about 70 nm to about 80 nm, from about 80 nm to about 100 nm, from about 80 nm to about 90 nm, or from about 90 nm to about 100 nm. In some embodiments, the average LNP diameter of the LNP formulation may be from about 70 nm to about 100 nm. In a particular embodiment, the average LNP diameter of the LNP formulation may be about 80 nm. In some embodiments, the average LNP diameter of the LNP formulation may be about 100 nm. In some embodiments, the average LNP diameter of the LNP formulation ranges from about 1 mm to about 500 mm, from about 5 mm to about 200 mm, from about 10 mm to about 100 mm, from about 20 mm to about 80 mm, from about 25 mm to about 60 mm, from about 30 mm to about 55 mm, from about 35 mm to about 50 mm, or from about 38 mm to about 42 mm.

A LNP may, in some instances, be relatively homogenous. A polydispersity index may be used to indicate the homogeneity of a LNP, e.g., the particle size distribution of the lipid nanoparticles. A small (e.g., less than 0.3) polydispersity index generally indicates a narrow particle size distribution. A LNP may have a polydispersity index from about 0 to about 0.25, such as 0.01, 0.02, 0.03, 0.04, 0.05, 0.06, 0.07, 0.08, 0.09, 0.10, 0.11, 0.12, 0.13, 0.14, 0.15, 0.16, 0.17, 0.18, 0.19, 0.20, 0.21, 0.22, 0.23, 0.24, or 0.25. In some embodiments, the polydispersity index of a LNP may be from about 0.10 to about 0.20.

The zeta potential of a LNP may be used to indicate the electrokinetic potential of the composition. In some embodiments, the zeta potential may describe the surface charge of a LNP. Lipid nanoparticles with relatively low charges, positive or negative, are generally desirable, as more highly charged species may interact undesirably with cells, tissues, and other elements in the body. In some embodiments, the zeta potential of a LNP may be from about −10 mV to about +20 mV, from about −10 mV to about +15 mV, from about −10 mV to about +10 mV, from about −10 mV to about +5 mV, from about −10 mV to about 0 mV, from about −10 mV to about −5 mV, from about −5 mV to about +20 mV, from about −5 mV to about +15 mV, from about −5 mV to about +10 mV, from about −5 mV to about +5 mV, from about −5 mV to about 0 mV, from about 0 mV to about +20 mV, from about 0 mV to about +15 mV, from about 0 mV to about +10 mV, from about 0 mV to about +5 mV, from about +5 mV to about +20 mV, from about +5 mV to about +15 mV, or from about +5 mV to about +10 mV.

The efficiency of encapsulation of a protein and/or nucleic acid, e.g., Gene Writer polypeptide or mRNA encoding the polypeptide, describes the amount of protein and/or nucleic acid that is encapsulated or otherwise associated with a LNP after preparation, relative to the initial amount provided. The encapsulation efficiency is desirably high (e.g., close to 100%). The encapsulation efficiency may be measured, for example, by comparing the amount of protein or nucleic acid in a solution containing the lipid nanoparticle before and after breaking up the lipid nanoparticle with one or more organic solvents or detergents. An anion exchange resin may be used to measure the amount of free protein or nucleic acid (e.g., RNA) in a solution. Fluorescence may be used to measure the amount of free protein and/or nucleic acid (e.g., RNA) in a solution. For the lipid nanoparticles described herein, the encapsulation efficiency of a protein and/or nucleic acid may be at least 50%, for example 50%, 55%, 60%, 65%, 70%, 75%, 80%, 85%, 90%, 91%, 92%, 93%, 94%, 95%, 96%, 97%, 98%, 99%, or 100%. In some embodiments, the encapsulation efficiency may be at least 80%. In some embodiments, the encapsulation efficiency may be at least 90%. In some embodiments, the encapsulation efficiency may be at least 95%.

A LNP may optionally comprise one or more coatings. In some embodiments, a LNP may be formulated in a capsule, film, or table having a coating. A capsule, film, or tablet including a composition described herein may have any useful size, tensile strength, hardness or density.

Additional exemplary lipids, formulations, methods, and characterization of LNPs are taught by WO2020061457, which is incorporated herein by reference in its entirety.

In some embodiments, in vitro or ex vivo cell lipofections are performed using Lipofectamine MessengerMax (Thermo Fisher) or TransIT-mRNA Transfection Reagent (Mirus Bio). In certain embodiments, LNPs are formulated using the GenVoy_ILM ionizable lipid mix (Precision NanoSystems). In certain embodiments, LNPs are formulated using 2,2-dilinoleyl-4-dimethylaminoethyl-[1,3]-dioxolane (DLin-KC2-DMA) or dilinoleylmethyl-4-dimethylaminobutyrate (DLin-MC3-DMA or MC3), the formulation and in vivo use of which are taught in Jayaraman et al. Angew Chem Int Ed Engl 51(34):8529-8533 (2012), incorporated herein by reference in its entirety.

LNP formulations optimized for the delivery of CRISPR-Cas systems, e.g., Cas9-gRNA RNP, gRNA, Cas9 mRNA, are described in WO2019067992 and WO2019067910, both incorporated by reference.

Additional specific LNP formulations useful for delivery of nucleic acids are described in U.S. Pat. Nos. 8,158,601 and 8,168,775, both incorporated by reference, which include formulations used in patisiran, sold under the name ONPATTRO.

Exemplary dosing of Gene Writer LNP may include about 0.1, 0.25, 0.3, 0.5, 1, 2, 3, 4, 5, 6, 8, 10, or 100 mg/kg (RNA). Exemplary dosing of AAV comprising a nucleic acid encoding one or more components of the system may include an MOI of about 10¹¹, 10¹², 10¹³, and 1014 vg/kg.

In some embodiments, a lipid nanoparticle (or a formulation comprising lipid nanoparticles) lacks reactive impurities (e.g., aldehydes or ketones), or comprises less than a preselected level of reactive impurities (e.g., aldehydes or ketones). While not wishing to be bound by theory, in some embodiments, a lipid reagent is used to make a lipid nanoparticle formulation, and the lipid reagent may comprise a contaminating reactive impurity (e.g., an aldehyde or ketone). A lipid regent may be selected for manufacturing based on having less than a preselected level of reactive impurities (e.g., aldehydes or ketones). Without wishing to be bound by theory, in some embodiments, aldehydes can cause modification and damage of RNA, e.g., cross-linking between bases and/or covalently conjugating lipid to RNA (e.g., forming lipid-RNA adducts). This may, in some instances, lead to failure of a reverse transcriptase reaction and/or incorporation of inappropriate bases, e.g., at the site(s) of lesion(s), e.g., a mutation in a newly synthesized target DNA.

In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, a lipid nanoparticle formulation is produced using a lipid reagent comprising less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, a lipid nanoparticle formulation is produced using a lipid reagent comprising: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation is produced using a plurality of lipid reagents, and each lipid reagent of the plurality independently meets one or more criterion described in this paragraph. In some embodiments, each lipid reagent of the plurality meets the same criterion, e.g., a criterion of this paragraph.

In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, the lipid nanoparticle formulation comprises less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, the lipid nanoparticle formulation comprises: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content. In some embodiments, one or more, or optionally all, of the lipid reagents used for a lipid nanoparticle as described herein or a formulation thereof comprise less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species. In some embodiments, one or more, or optionally all, of the lipid reagents used for a lipid nanoparticle as described herein or a formulation thereof comprise: (i) less than 5%, 4%, 3%, 2%, 1%, 0.9%, 0.8%, 0.7%, 0.6%, 0.5%, 0.4%, 0.3%, 0.2%, or 0.1% total reactive impurity (e.g., aldehyde) content; and (ii) less than 5%, 4%, 3%, 2%, 1%, 0.9%, 0.8%, 0.7%, 0.6%, 0.5%, 0.4%, 0.3%, 0.2%, or 0.1% of any single reactive impurity (e.g., aldehyde) species.

In some embodiments, total aldehyde content and/or quantity of any single reactive impurity (e.g., aldehyde) species is determined by liquid chromatography (LC), e.g., coupled with tandem mass spectrometry (MS/MS), e.g., according to the method described in Example 26. In some embodiments, reactive impurity (e.g., aldehyde) content and/or quantity of reactive impurity (e.g., aldehyde) species is determined by detecting one or more chemical modifications of a nucleic acid molecule (e.g., an RNA molecule, e.g., as described herein) associated with the presence of reactive impurities (e.g., aldehydes), e.g., in the lipid reagents. In some embodiments, reactive impurity (e.g., aldehyde) content and/or quantity of reactive impurity (e.g., aldehyde) species is determined by detecting one or more chemical modifications of a nucleotide or nucleoside (e.g., a ribonucleotide or ribonucleoside, e.g., comprised in or isolated from a template nucleic acid, e.g., as described herein) associated with the presence of reactive impurities (e.g., aldehydes), e.g., in the lipid reagents, e.g., as described in Example 27. In embodiments, chemical modifications of a nucleic acid molecule, nucleotide, or nucleoside are detected by determining the presence of one or more modified nucleotides or nucleosides, e.g., using LC-MS/MS analysis, e.g., as described in Example 27.

In some embodiments, a nucleic acid (e.g., RNA) described herein (e.g., a template nucleic acid or a nucleic acid encoding a GeneWriter) does not comprise an aldehyde modification, or comprises less than a preselected amount of aldehyde modifications. In some embodiments, on average, a nucleic acid has less than 50, 20, 10, 5, 2, or 1 aldehyde modifications per 1000 nucleotides, e.g., wherein a single cross-linking of two nucleotides is a single aldehyde modification. In some embodiments, the aldehyde modification is an RNA adduct (e.g., a lipid-RNA adduct). In some embodiments, the aldehyde-modified nucleotide is cross-linking between bases. In some embodiments, a nucleic acid (e.g., RNA) described herein comprises less than 50, 20, 10, 5, 2, or 1 cross-links between nucleotide.

### Retargeting

Retargeting (e.g., of a Gene Writer polypeptide or nucleic acid molecule, or of a system as described herein) may comprise directing the polypeptide to bind at the target site. In some embodiments, the recombinase domain of the polypeptide is also modified as described.

### Gene Writer Polypeptide Determinants

In some embodiments, a Gene Writer polypeptide comprises a modification to a DNA-binding domain, e.g., relative to the wild-type polypeptide. In some embodiments, the DNA-binding domain comprises an addition, deletion, replacement, or modification to the amino acid sequence of the original DNA-binding domain. In some embodiments, the DNA-binding domain is modified to include a heterologous functional domain that binds specifically to a target nucleic acid (e.g., DNA) sequence of interest. In some embodiments, the functional domain replaces at least a portion (e.g., the entirety of) the prior DNA-binding domain of the polypeptide. In some embodiments, the functional domain comprises a zinc finger (e.g., a zinc finger that specifically binds to the target nucleic acid (e.g., DNA) sequence of interest. In some embodiments, the functional domain comprises a Cas domain (e.g., a Cas domain that specifically binds to the target nucleic acid (e.g., DNA) sequence of interest. In embodiments, the Cas domain comprises a Cas9 or a mutant or variant thereof (e.g., as described herein). In embodiments, the Cas domain is associated with a guide RNA (gRNA), e.g., as described herein. In embodiments, the Cas domain is directed to a target nucleic acid (e.g., DNA) sequence of interest by the gRNA. In embodiments, the Cas domain is encoded in the same nucleic acid (e.g., RNA) molecule as the gRNA. In embodiments, the Cas domain is encoded in a different nucleic acid (e.g., RNA) molecule from the gRNA.

All publications, patent applications, patents, and other publications and references (e.g., sequence database reference numbers) cited herein are incorporated by reference in their entirety. For example, all GenBank, Unigene, and Entrez sequences referred to herein, e.g., in any Table herein, are incorporated by reference. Unless otherwise specified, the sequence accession numbers specified herein, including in any Table herein, refer to the database entries current as of May 26, 2021. When one gene or protein references a plurality of sequence accession numbers, all of the sequence variants are encompassed.

### EXAMPLES

The invention is further illustrated by the following examples. The examples are provided for illustrative purposes only and are not to be construed as limiting the scope or content of the invention in any way.

### Example 1: Delivery of a Gene Writer™ System to Mammalian Cells

This example describes a Gene Writer™ genome editing system delivered to a mammalian cell for site-specific insertion of exogenous DNA into a mammalian cell genome.

In this example, the polypeptide component of the Gene Writer™ system is a recombinase protein, e.g., a recombinase protein comprising an amino acid sequence of any of SEQ ID NOs: 1-12,677 (e.g., SEQ ID NOs: 1-11,432), and the template DNA component is a plasmid DNA that comprises a target recombination site, e.g., a recognition sequence occurring within a nucleotide sequence of the LeftRegion or RightRegion, e.g., a LeftRegion or RightRegion comprising a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively.

HEK293T cells are transfected with the following test agents:
1. Scrambled DNA control
2. DNA coding for the polypeptide described above
3. Template DNA described above
4. Combination of 2 and 3

After transfection, HEK293T cells are cultured for at least 4 days and then assayed for site-specific genome editing. Genomic DNA is isolated from each group of HEK293 cells. PCR is conducted with primers that flank the appropriate sequence or genomic locus. The PCR product is run on an agarose gel to measure the length of the amplified DNA.

A PCR product of the expected length, indicative of a successful Gene Writing™ genome editing event that inserts the DNA plasmid template into the target genome, is observed only in cells that were transfected with the complete Gene Writer™ system of group 4 above.

### Example 2: Targeted Delivery of a Gene Expression Unit into Mammalian Cells Using a Gene Writer™ System

This example describes the making and using of a Gene Writer genome editor to insert a heterologous gene expression unit into the mammalian genome.

In this example, a recombinase protein, e.g., a recombinase protein comprising an amino acid sequence of any of SEQ ID NOs: 1-12,677 (e.g., SEQ ID NOs: 1-11,432). The recombinase protein targets an appropriate genomic copy of a recognition sequence of the recombinase polypeptide for DNA integration. The template DNA component is a plasmid DNA that comprises a target recombination site (a recognition sequence occurring within a nucleotide sequence in the LeftRegion or RightRegion, e.g., a LeftRegion or RightRegion comprising a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively) and gene expression unit. A gene expression unit comprises at least one regulatory sequence operably linked to at least one coding sequence. In this example, the regulatory sequences include the CMV promoter and enhancer, an enhanced translation element, and a WPRE. The coding sequence is the GFP open reading frame.

HEK293 cells are transfected with the following test agents:
1. Scrambled DNA control
2. DNA coding for the polypeptide described above
3. Template DNA described above
4. Combination of 2 and 3

After transfection, HEK293 cells are cultured for at least 4 days and assayed for site-specific Gene Writing genome editing. Genomic DNA is isolated from the HEK293 cells and PCR is conducted with primers that flank the target integration site in the genome. The PCR product is run on an agarose gel to measure the length of DNA. A PCR product of the expected length, indicative of a successful Gene Writing™ genome editing event, is detected in cells transfected with the test agent of group 4 (complete Gene Writer™ system).

The transfected cells are cultured for a further 10 days, and after multiple cell culture passages are assayed for GFP expression via flow cytometry. The percent of cells that are GFP positive from each cell population are calculated. GFP positive cells are detected in the population of HEK293 cells that were transfected with group 4 test agent, demonstrating that a gene expression unit added into the mammalian cell genome via Gene Writing genome editing is expressed.

### Example 3: Targeted Delivery of a Splice Acceptor Unit into Mammalian Cells Using a Gene Writer™ System

This example describes the making and use of a Gene Writing genome editing system to add a heterologous sequence into an intronic region to act as a splice acceptor for an upstream exon. Splicing into the first intron a new exon containing a splice acceptor site at the 5′ end and a polyA tail at the 3′ end will result in a mature mRNA containing the first natural exon of the natural locus spliced to the new exon.

In this example, a recombinase protein, e.g., a recombinase protein comprising an amino acid sequence of any of SEQ ID NOs: 1-12,677 (e.g., SEQ ID NOs: 1-11,432). The recombinase protein targets a compatible recognition site in a genome, e.g., a HEK293 genome, for DNA integration. The template DNA codes for GFP with a splice acceptor site immediately 5′ to the first amino acid of mature GFP (the start codon is removed) and a 3′ polyA tail downstream of the stop codon.

HEK293 cells are transfected with the following test agents:
1. Scrambled DNA control
2. DNA coding for the polypeptide described above
3. Template DNA described above
4. Combination of 2 and 3

After transfection, HEK293 cells are cultured for at least 4 days and assayed for site-specific Gene Writing genome editing and appropriate mRNA processing. Genomic DNA is isolated from the HEK293 cells. Reverse transcription-PCR is conducted to measure the mature mRNA containing the first natural exon of the target locus and the new exon. The RT-PCR reaction is conducted with forward primers that bind to the target locus (e.g., the first natural exon of the target locus) and with reverse primers that bind to GFP. The RT-PCR product is run on an agarose gel to measure the length of DNA. A PCR product of the expected length is detected in cells transfected with the test agent of group 4, indicative of a successful Gene Writing genome editing event and a successful splice event. This result would demonstrate that a Gene Writing genome editing system can add a heterologous sequence encoding a gene into a target locus, e.g., intronic region, to act as a splice acceptor for the upstream exon.

The transfected cells are cultured for a further 10 days and, after multiple cell culture passages, are assayed for GFP expression via flow cytometry. The percent of cells that are GFP positive from each cell population are calculated. GFP positive cells are detected in the population of HEK293 cells that were transfected with group 4 test agent, demonstrating that a gene expression unit added into the mammalian cell genome via Gene Writing genome editing is expressed.

### Example 4: Specificity of Gene Writing in Mammalian Cells

This example describes a Gene Writer™ genome system delivered to a mammalian cell for site-specific insertion of exogenous DNA into a mammalian cell genome and a measurement of the specificity of the site-specific insertion.

In this example, Gene Writing is conducted in HEK293T cells as described in any of the preceding Examples. After transfection, HEK293T cells are cultured for at least 4 days and then assayed for site-specific genome editing. Linear amplification PCR is conducted as described in Schmidt et al. Nature Methods 4, 1051-1057 (2007) using a forward primer specific to the template DNA that will amplify adjacent genomic DNA. Amplified PCR products are then sequenced using next generation sequencing technology on a MiSeq instrument. The MiSeq reads are mapped to the HEK293T genome to identify integration sites in the genome.

The percent of LAM-PCR sequencing reads that map to the target genomic site is the specificity of the Gene Writer.

The number of total genomic sites that LAM-PCR sequencing reads map to is the number of total integration sites.

### Example 5: Efficiency of Gene Writing in Mammalian Cells

This example describes Gene Writer™ genome system delivered to a mammalian cell for site-specific insertion of exogenous DNA into a mammalian cell genome, and a measurement of the efficiency of Gene Writing.

In this example, Gene Writing is conducted in HEK293T cells as described in any of the preceding Examples. After transfection, HEK293T cells are cultured for at least 4 days and then assayed for site-specific genome editing. Digital droplet PCR is conducted as described in Lin et al., Human Gene Therapy Methods 27(5), 197-208, 2016. A forward primer binds to the template DNA and a reverse primer binds on one side of the appropriate genomic integration site, thus a PCR amplification is only expected upon integration of target DNA. A probe to the target site containing a FAM fluorophore and is used to measure the number of copies of the target DNA in the genome. Primers and HEX-fluorophore probe specific to a housekeeping gene (e.g. RPP30) are used to measure the copies of genomic DNA per droplet.

The copy number of target DNA per droplet normalized to the copy number of house keeping DNA per droplet is the efficiency of the Gene Writer.

### Example 6: Determination of Copy Number of a Recombinase in a Cell

The following example describes the absolute quantification of a recombinase on a per cell basis. This measurement is performed using the AQUA mass spectrometry based methods, e.g., as accessible at the following uniform resource locator (URL):https://www.sciencedirect.com/science/article/pii/S1046202304002087? via %3Dihub

Following delivery of the recombinase and DNA template to the cells, the recombination is allowed to proceed for 24 hours after which the cells are quantified and then quantified by this MS method. This method involves two stages.

In the first stage, the amino acid sequence of the recombinase is examined, and a representative tryptic peptide is selected for analysis. An AQUA peptide is then synthesized with an amino acid sequence that exactly mimics the corresponding native peptide produced during proteolysis. However, stable isotopes are incorporated at one residue to allow the mass spectrometer to differentiate between the analyte and internal standard. The synthetic peptide and the native peptide share the same physicochemical properties including chromatographic co-elution, ionization efficiency, and relative distributions of fragment ions, but are differentially detected in a mass spectrometer due to their mass difference. The synthetic peptide is next analyzed by LC-MS/MS techniques to confirm the retention time of the peptide, determine fragment ion intensities, and select an ion for SRM analysis. In such an SRM experiment, a triple quadrupole mass spectrometer is directed to select the expected precursor ion in the first scanning quadrupole, or Q1. Only ions with this one mass-to-charge (m/z) ratio are directed into the collision cell (Q2) to be fragmented. The resulting product ions are passed to the third quadrupole (Q3), where the m/z ratio for single fragment ion is monitored across a narrow m/z window.

The second stage involves quantification of the recombinase from cell or tissue lysates. A quantified number of cells or mass of tissue is used to initiate the reaction and is used to normalize the quantification to a per cell basis. Cell lysates are separated prior to proteolysis to increase the dynamic range of the assay via SDS-PAGE, followed by excision of the region of the gel where the recombinase migrates. In-gel digestion is performed to obtain native tryptic peptides. In-gel digestion is performed in the presence of the AQUA peptide, which is added to the gel pieces during the digestion process. Following proteolysis, the complex peptide mixture, containing both heavy and light peptides, is analyzed in an LC-SRM experiment using parameters determined during the first stage.

The results of the mass spectrometry-based quantification is converted to a number of proteins loaded to determine the number of recombinases per cell.

### Example 7: Copy Number of DNA Inside Cell

### Q-FISH

The following example describes the quantification of delivered DNA template on a per cell basis. In this example the DNA that the recombinase is integrating contains a DNA-probe binding site. Following delivery of the recombinase and DNA template to the cells, the recombination is allowed to proceed for 24 hours, after which the cells are quantified and are prepared for quantitative fluorescence in situ hybridization (Q-FISH). Q-FISH is conducted using FISH Tag DNA Orange Kit, with Alex Fluor 555 dye (ThermoFisher catalog number F32948). Briefly, a DNA probe that binds to the DNA-probe binding site on the DNA template is generated through a procedure of nick translation, dye labeling, and purification as described in the Kit manual. The cells are then labeled with the DNA probe as described in the Kit manual. The cells are imaged on a Zeiss LSM 710 confocal microscope with a 63× oil immersion objective while maintained at 37° C. and 5% CO2. The DNA probe is subjected to 555 nm laser excitation to stimulate Alexa Flour. A MATLAB script is written to measure the Alex Fluor intensity relative to a standard generated with known quantities of DNA. Using this method, the amount of template DNA delivered to a cell is determined.

qPCR

The following example describes the quantification of delivered DNA template on a per cell basis. In this example the DNA that the recombinase is integrating contains a DNA-probe binding site. Following delivery of the recombinase and DNA template to the cells, the recombination is allowed to proceed for 24 hours after which the cells are quantified, and cells are prepared for quantitative PCR (qPCR). qPCR is conducted using standard kits for this protocol, such as the ThermoFisher TaqMan product (https://www.thermofisher.com/us/en/home/life-science/pcr/real-time-pcr/real-time-pcr-assays-search.html). Briefly, primers are designed that specifically amplify a region of the delivered template DNA as well as probes for the specific amplicon. A standard curve is generated by using a serial dilution of quantified pure template DNA to correlate threshold Ct numbers to number of DNA templates. The DNA is then extracted from the cells being analyzed and input into the qPCR reaction along with all additional components per the manufacturer's directions. The samples are than analyzed on an appropriate qPCR machine to determine the Ct number, which is then mapped to the standard curve for absolute quantification. Using this method, the amount of template DNA delivered to a cell is determined.

### Example 8: Intracellular Ratio of DNA:Recombinase

The following example describes the determination of the ratio of recombinase protein to template DNA cell in the target cells. Following delivery of the recombinase and DNA template to the cells, the recombination is allowed to proceed for 24 hours after which the cells are quantified, and cells are prepared quantification of the recombinase and of the template DNA as outlined in the above examples. These two values (recombinase per cell and template DNA per cell) are then divided (recombinase per cell/template DNA per cell) to determine the bulk average ratio of these quantities. Using this method, the ratio of recombinase to template DNA delivered to a cell is determined.

### Example 9: Activity in Presence of DNA-Damage Response Inhibiting Agents—Activity in Presence of NHEJ Inhibitor

The following example describes the assaying of activity of the recombinase protein in the presence of inhibitors of non-homologous end joining to highlight the lack of dependence on the expression of the proteins involved in these pathways for activity of the recombinase. Briefly, the assay outlined to determine efficiency of recombinase activity outlined in the example above is performed. However, in this case two separate experiments are performed.

In experiment 1, 24 hours after delivery of the recombinase and Template DNA, 1 M of the NHEJ inhibitor Scr7 (https://www.sigmaaldrich.com/catalog/product/sigma/sml1546?lang=en&region=US) is added to the cell growth media to inhibit this pathway. All other elements of the protocol are identical.

In experiment 2, the cells are manipulated identically as in experiment 1 but no inhibitor is added to the media. Both experiments are analyzed for efficiency per the example above and the % inhibited activity relative to uninhibited activity is determined.

### Example 10: Activity in Presence of DNA-Damage Response Inhibiting Agents—Activity in Presence of HDR Inhibitor

The following example describes the assaying of activity of the recombinase protein in the presence of inhibitors of homologous recombination to highlight the lack of dependence on the expression of the proteins involved in these pathways for activity of the recombinase. Briefly, the assay outlined to determine efficiency of recombinase activity outlined in the example above is performed. However, in this case, two separate experiments are performed.

In experiment 1: 24 hours after delivery of the recombinase and Template DNA, 1 M of the HR inhibitor B02 (https://www.selleckchem.com/products/b02.html) is added to the cell growth media to inhibit this pathway. All other elements of the protocol are identical.

In experiment 2: the cells are manipulated identically as in experiment 1 but no inhibitor is added to the media. Both experiments are analyzed for efficiency per the example above and the % inhibited activity relative to uninhibited activity is determined.

### Example 11: Percentage of Nuclear Versus Cytoplasmic Recombinase

The following example describes the determination of the ratio of recombinase protein in the nucleus vs the cytoplasm of target cells. 12 hours following delivery of the recombinase and DNA template to the cells as described herein, the cells are quantified and prepared for analysis. The cells are split into nuclear and cytoplasmic fractions using the following standard kits, following manufacturer directions: NE-PER Nuclear and Cytoplasmic Extraction by ThermoFisher. Both the cytoplasmic and nuclear fractions are kept and then put through the mass spec based recombinase quantification assay outlined in the example above. Using this method, the ratio of nuclear recombinase to cytoplasmic recombinase in the cells is determined.

### Example 12: Delivery to Plant Cells

This example illustrates a method of delivering at least one recombinase to a plant cell wherein the plant cell is located in a plant or plant part. More specifically, this example describes delivery of a Gene Writing recombinase and its template DNA to a non-epidermal plant cell (i.e., a cell in a soybean embryo), in order to edit an endogenous plant gene (i.e., phytoene desaturase, PDS) in germline cells of excised soybean embryos. This example describes delivery of polynucleotides encoding the delivered transgene through multiple barriers (e.g., multiple cell layers, seed coat, cell walls, plasma membrane) directly into soybean germline cells, resulting in a heritable alteration of the target nucleotide sequence, PDS. The methods described do not employ the common techniques of bacterially mediated transformation (e.g., by Agrobacterium sp.) or biolistics.

Plasmids are designed for delivery of recombinase and a single template DNA targeting the endogenous phytoene desaturase (PDS) in soybean (Glycine max). It will be apparent to one skilled in the art that analogous plasmids are easily designed to encode other recombinases and template DNA sequences, optionally including different elements (e. g., different promoters, terminators, selectable or detectable markers, a cell-penetrating peptide, a nuclear localization signal, a chloroplast transit peptide, or a mitochondrial targeting peptide, etc.), and used in a similar manner.

In a first series of experiments, these vectors are delivered to non-epidermal plant cells in soybean embryos using combinations of delivery agents and electroporation. Mature, dry soybean seeds (cv. Williams 82) are surface-sterilized as follows. Dry soybean seeds are held for 4 hours in an enclosed chamber holding a beaker containing 100 milliliters 5% sodium hypochlorite solution to which 4 milliliters hydrochloric acid are freshly added. Seeds remain desiccated after this sterilization treatment. The sterilized seeds are split into 2 halves by manual application of a razor blade and the embryos are manually separated from the cotyledons. Each test or control treatment is carried out on 20 excised embryos. The following series of experiments is then performed.

Experiment 1: A delivery solution containing the vectors (100 nanograms per microliter of each plasmid) in 0.01% CTAB (cetyltrimethylammonium bromide, a quaternary ammonium surfactant) in sterile-filtered milliQ water is prepared. Each solution is chilled to 4 degrees Celsius and 500 microliters are added directly to the embryos, which are then immediately placed on ice in a vacuum chamber and subjected to a negative pressure (2×10″3 millibar) treatment for 15 minutes. Following the chilling/negative pressure treatments, the embryos are treated with electric current using a BTX-Harvard ECM-830 electroporation device set with the following parameters: 50V, 25 millisecond pulse length, 75 millisecond pulse interval for 99 pulses.
Experiment 2: conditions identical to Experiment 1, except that the initial contacting with delivery solution and negative pressure treatments are carried out at room temperature.
Experiment 3: conditions identical to Experiment 1, except that the delivery solution is prepared without CTAB but includes 0.1% Silwet L-77™ (CAS Number 27306-78-1, available from Momentive Performance Materials, Albany, N.Y). Half (10 of 20) of the embryos receiving each treatment undergo electroporation, and the other half of the embryos do not.
Experiment 4: conditions identical to Experiment 3, except that several delivery solutions are prepared, where each further includes 20 micrograms/milliliter of one single-walled carbon nanotube preparation selected from those with catalogue numbers 704113, 750530, 724777, and 805033, all obtainable from Sigma-Aldrich, St. Louis, MO. Half (10 of 20) of the embryos receiving each treatment undergo electroporation, and the other half of the embryos do not.
Experiment 5: conditions identical to Experiment 3, except that the delivery solution further includes 20 micrograms/milliliter of triethoxylpropylaminosilane-functionalized silica nanoparticles (catalogue number 791334, Sigma-Aldrich, St. Louis, MO. Half (10 of 20) of the embryos receiving each treatment undergo electroporation, and the other half of the embryos do not.
Experiment 6: conditions identical to Experiment 3, except that the delivery solution further includes 9 micrograms/milliliter branched polyethylenimine, molecular weight −25,000 (CAS Number 9002-98-6, catalogue number 408727, Sigma-Aldrich, St. Louis, MO) or 9 micro grams/milliliter branched polyethylenimine, molecular weight −800 (CAS Number 25987-06-8, catalogue number 408719, Sigma-Aldrich, St. Louis, MO). Half (10 of 20) of the embryos receiving each treatment undergo electroporation, and the other half of the embryos do not.
Experiment 7: conditions identical to Experiment 3, except that the delivery solution further includes 20% v/v dimethylsulf oxide (DMSO, catalogue number D4540, Sigma-Aldrich, St. Louis, MO). Half (10 of 20) of the embryos receiving each treatment undergo electroporation, and the other half of the embryos do not.
Experiment 8: conditions identical to Experiment 3, except that the delivery solution further contains 50 micromolar nono-arginine (RRRRRRRRR). Half (10 of 20) of the embryos receiving each treatment undergo electroporation, and the other half of the embryos do not.
Experiment 9: conditions identical to Experiment 3, except that following the vacuum treatment, the embryos and treatment solutions are transferred to microcentrifuge tubes and centrifuged 2, 5, 10, or 20 minutes at 4000×g. Half (10 of 20) of the embryos receiving each treatment undergo electroporation, and the other half of the embryos do not.
Experiment 10: conditions identical to Experiment 3, except that following the vacuum treatment, the embryos and treatment solutions are transferred to microcentrifuge tubes and centrifuged 2, 5, 10, or 20 minutes at 4000×g.
Experiment 11: conditions identical to Experiment 4, except that following the vacuum treatment, the embryos and treatment solutions are transferred to microcentrifuge tubes and centrifuged 2, 5, 10, or 20 minutes at 4000×g.
Experiment 12: conditions identical to Experiment 5, except that following the vacuum treatment, the embryos and treatment solutions are transferred to microcentrifuge tubes and centrifuged 2, 5, 10, or 20 minutes at 4000×g.

After the delivery treatment, each treatment group of embryos is washed 5 times with sterile water, transferred to a petri dish containing 12 MS solid medium (2.165 g Murashige and Skoog medium salts, catalogue number MSP0501, Caisson Laboratories, Smithfield, UT), 10 grams sucrose, and 8 grams Bacto agar, made up to 1.00 liter in distilled water), and placed in a tissue culture incubator set to 25 degrees Celsius. After the embryos have elongated, developed roots and true leaves have emerged, the seedlings are transferred to soil and grown out. Modification of all endogenous PDS alleles results in a plant unable to produce chlorophyll and having a visible bleached phenotype. Modification of a fraction of all endogenous PDS alleles results in plants still able to produce chlorophyll; plants that are heterozygous for an altered PDS gene will are grown out to seed and the efficiency of heritable genome modification is determined by molecular analysis of the progeny seeds.

### Example 13: Recombinase-Mediated Plasmid Integration in Human Cells

This example describes the use of a serine recombinase-based Gene Writer system for the targeted integration of a template DNA into the human genome. More specifically, this example describes the transfection of a two plasmid system into HEK293T cells for in vitro Gene Writing, e.g., as a means of evaluating a new Gene Writing polypeptide for integration activity in human cells.

Briefly, a two plasmid system was designed, comprising: 1) an integrase expression plasmid, e.g., a plasmid encoding a human codon optimized serine integrase, e.g., a serine integrase comprising an amino acid sequence of any of SEQ ID NOs: 1-12,677 (e.g., SEQ ID NOs: 1-11,432), driven by the mammalian CMV promoter, and 2) a template plasmid, e.g., a plasmid comprising (i) a sequence comprising the recognition site of a serine integrase, e.g., a ˜500 bp sequence from the endogenous flanking region of a serine integrase, e.g., a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432); (ii) a promoter for expression in mammalian cells, e.g., a CMV promoter; (iii) a reporter gene whose expression is controlled by (ii), e.g., an EGFP gene; (iv) a self-cleaving polypeptide, e.g., a T2A peptide; (v) a marker enabling selection in mammalian cells, e.g., a puromycin resistance gene; and (vi) a termination signal, e.g., a poly A tail. Without wishing to be bound by theory, some embodiments of the template plasmid may comprise elements occurring in the orientation (i), (ii), (iii), (iv), (v).

To deliver the Gene Writer system into HEK293T cells, ˜120,000 cells were transfected with either: (1) 50 ng template plasmid and 225 ng transfection balance plasmid (template only control); or (2) 50 ng template plasmid, 25 ng integrase expression plasmid, and 225 ng transfection balance plasmid, using TransIT-293 Reagent (Mirusbio) according to manufacturer's instructions. Three days post-transfection, the efficiency of delivery was measured using flow cytometry to determine the percentage of GFP positive cells. Cells were split between days 3 and 13 of the time course experiments. Between day 13 and day 27, transfected cells that had been split were maintained in one of two conditions: 1) a subset of the cells were maintained in normal cell culture medium and flow cytometry was performed every 3˜4 days to determine the GFP expression from successfully integrated template; 2) a subset of the cells were maintained in medium supplemented with 1 g/mL puromycin, where the puromycin resistant cells were harvested after ˜2 weeks of selection. In some instances, a Gene Writer system that demonstrated activity in human cells resulted in detectable reporter expression in at least 3% of cells at day 21, e.g., detectable expression of GFP in at least 3% of cells as determined by flow cytometry. In some instances, a Gene Writer system that demonstrated activity in human cells resulted in detectable reporter expression in a percentage of cells that was greater than demonstrated with a template only control, e.g., higher as compared to transfection condition (1), e.g., at least 2, 3, 4, 5, 6, 7, 8, 9, 10, 20, 30, 40, 50, 60, 70, 80, 90, 100, 200, 300, 400, 500, 600, 700, 800, 900, or 1000-fold higher compared to a template only control.

To determine the integration site used by an active Gene Writer, the parallel cultures being maintained under puromycin selection were harvested for genomic isolation and analyzed by a unidirectional sequencing assay, as described herein in Example 18.

As shown in Table 22 below, Gene Writer polypeptides, e.g., serine recombinases comprising an amino acid sequence of any of (e.g., SEQ ID NOs: 1-11,432), were assayed for integration of a template DNA comprising a GFP expression cassette and a recognition sequence, e.g., a recognition sequence of SEQ ID NOs: 13,001-25,677 (e.g., SEQ ID NOs: 13,001-24,432) or SEQ ID NOs: 26,001-38,677 (e.g., SEQ ID NOs: 26,001-37,432), in human cells (see Example 13).

TABLE 22
Screening data for recombinase-mediated integration in human cells

| Integrase | | | |
|---|---|---|---|
| A No. | FL58 Accession | Int ID | % GFP+ |
| 25 | NC_001978.3::NP_047974.1 | PhiC31 | 0.915333333 |
| 102 | KY030782.1::APD21144.1 | Int1 | 0 |
| 128 | KP296792.1::AJK27795.1 | Int2 | 3.35 |
| 1200 | YP_459991.1 | Int3 | 7.19 |
| 162 | MF595878.1::ATB52753.1 | Int4 | 0 |
| 1201 | AAB51419.1 | Int5 | 0 |
| 1202 | AAF35174.1 | Int6 | 0.18 |
| 1203 | YP_006082695.1 | Int7 | 0.048 |
| 118 | AY657002.1::AAT72400.1 | Int8 | 0 |
| 511 | MK448811.1::QBX21446.1 | Int9 | 0 |
| 5 | NC_029073.1::YP_009223763.1 | Int10 | 0 |
| 336 | KP836355.1::AJW76937.1 | Int11 | 0 |
| 161 | NC_030947.1::YP_009276898.1 | Int12 | 0 |
| 146 | MG711462.1::AUV56418.1 | Int13 | 0 |
| 330 | KU230356.1::ALY07619.1 | Int14 | 0.38 |
| 1204 | YP_005549228.1 | Int15 | 0 |
| 1205 | YP_189066.1 | Int16 | 0 |
| 1206 | YP_005679179.1 | Int17 | 0.123 |
| 103 | KC595514.1::AGR47239.1 | Int18 | 1.966755 |
| 186 | KM983329.1::AJA42614.1 | Int19 | 0 |
| 6 | NC_018836.1::YP_006906230.1 | Int20 | 0 |
| 4 | NC_028746.1::YP_009193857.1 | Int21 | 0.22 |
| 1207 | YP_002804732.1 | Int22 | 0.16 |
| 19 | NC_021325.1::YP_008058952.1 | Int23 | 0.04 |
| 320 | MH552499.1::AXF52129.1 | Int24 | 0.09 |
| 22 | MK448667.1::QBX13795.1 | Int25 | 0 |
| 1208 | YP_001089468.1 | Int26 | 0 |
| 1209 | YP_001886479.1 | Int27 | 0 |
| 35 | KX522565.1::AOA49517.1 | Int28 | 0 |
| 477 | NC_003216.1::NP_463492.1 | Int29 | 0.91 |
| 436 | NC_025453.1::YP_009103095.1 | Int30 | 10.07333333 |
| 386 | MK448715.1::QBX16366.1 | Int31 | 0 |
| 46 | KM983332.1::AJA42824.1 | Int32 | 0.31 |
| 1210 | BAA12435.1 | Int33 | 0 |
| 462 | JX507079.1::AFU62848.1 | Int34 | 0 |
| 1211 | YP_005759947.1 | Int35 | 0 |
| 458 | NC_019914.1::YP_007236569.1 | Int36 | 0.08 |
| 1212 | YP_004586821.1 | Int37 | 0 |
| 408 | NC_029069.1::YP_009223181.1 | Int38 | 6.3025 |
| 375 | MG969427.1::AVO22625.1 | Int39 | 0 |
| 1213 | YP_353073.2 | Int40 | 0 |
| 1214 | BAG46462.1 | Int41 | 0.05 |
| 434 | MK279899.1::AZS11727.1 | Int42 | 0.08 |
| 1215 | YP_006906969.1 | Int43 | 0.31 |
| 31 | KY676784.1::ARB11450.1 | Int44 | 0 |
| 43 | MG593803.1::AUG87323.1 | Int45 | 0.62 |
| 63 | MK937595.1::QDH92149.1 | Int46 | 0.61 |
| 147 | MH271298.1::AWY04899.1 | Int47 | 0 |
| 418 | MG593801.1::AUG87183.1 | Int48 | 0 |
| 142 | MK450421.1::QAX93309.1 | Int49 | 0 |
| 111 | NC_004682.2::NP_817623.1 | Int50 | 0 |
| 159 | KX641260.1::AOT24690.1 | Int51 | 2.005 |
| 1216 | YP_009031225.1 | Int52 | 0.111 |
| 202 | JF937108.1::AEK10337.2 | Int53 | 0 |
| 113 | MG944221.1::AVJ51390.1 | Int54 | 0.009 |
| 54 | KT124228.1::AKY03507.1 | Int55 | 0 |
| 96 | KC700556.1::AGM12072.1 | Int56 | 0.03 |
| 291 | NC_028960.2::YP_009214300.1 | Int57 | 0 |
| 295 | NC_022984.1::YP_008859055.1 | Int58 | 0 |
| 402 | MH834610.1::AYN57772.1 | Int59 | 0.04 |
| 27 | NC_004664.2::NP_813744.2 | Int60 | 0.17 |
| 23 | NC_018853.1::YP_006907228.1 | Int61 | 0 |
| 32 | MK686068.1::QBZ73369.1 | Int62 | 0.94 |
| 337 | MH155870.1::AWN05230.1 | Int63 | 0.11 |
| 67 | NC_031078.1::YP_009287835.1 | Int64 | 0 |
| 62 | NC_021560.1::YP_008130182.1 | Int65 | 0.29 |
| 491 | KT152029.1::AKY03358.1 | Int66 | 0.15 |
| 390 | MK392363.1::QAY15711.1 | Int67 | 0.24 |
| 304 | MH271308.1::AWY05745.1 | Int68 | 0 |
| 1217 | SGE40566.1 | Int69 | 0.05 |
| 335 | MF319184.1::ASR75970.1 | Int70 | 0.326 |
| 353 | JN243855.1::AEL19745.1 | Int71 | 0.61 |
| 28 | KT021004.1::ALA06428.1 | Int72 | 0 |
| 1218 | CBG73463.1 | Int73 | 0.038 |
| 435 | JN116825.1::AEV52018.1 | Int74 | 0.131 |
| 448 | MN096365.1::QDK02252.1 | Int75 | 0 |
| 331 | NC_007814.1::YP_512335.1 | Int76 | 2.53 |
| 332 | DQ221100.2::ABB55416.1 | Int77 | 1.71 |
| 37 | KY963371.1::ARW58518.1 | Int78 | 12.66 |
| 360 | KY963370.1::ARW58461.1 | Int79 | 9.36 |
| 338 | MK085976.1::AZF88373.1 | Int80 | 1.82 |
| 1219 | YP_001376196.1 | Int94 | 0.23 |
| 460 | JX887877.1::AFV15398.1 | Int95 | 4.89 |
| 474 | KF296717.1::AGV99364.1 | Int96 | 0.14 |
| 380 | MG727702.1::AUS03929.1 | Int97 | 0.93 |
| 1220 | AAD26564.1 | Int98 | 0 |
| 479 | KX190835.1::ANT40095.1 | Int99 | 0 |
| 461 | MF417928.1::ASN71601.1 | Int100 | 0.32 |
| 475 | MF417933.1::ASN71805.1 | Int101 | 21.03 |
| 478 | MH341451.1::AWN07855.1 | Int102 | 0.24 |
| 1221 | CAC97653.1 | Int103 | 0.22 |
| 1222 | CAD10281.2 | Int104 | 0.58 |
| 477 | NC_003216.1::NP_463492.1 | Int105 | 0.77 |
| 1223 | YP_004301563.1 | Int106 | 1.77 |
| 1224 | YP_006538656.1 | Int107 | 0 |
| 1225 | YP_006685721.1 | Int108 | 0 |
| 982 | NZ_CP030772.1::WP_138968117.1 | Int109 | 0.13 |
| 983 | NZ_CP011275.1::WP_082859072.1 | Int110 | 0.52 |
| 984 | NC_019309.1::YP_006962361.1 | Int111 | 0.17 |
| 985 | NZ_CP029174.1::WP_108943154.1 | Int112 | 0.57 |
| 986 | NZ_CP032696.1::WP_120708991.1 | Int113 | 0.2 |
| 987 | NC_011758.1::WP_012606065.1 | Int114 | 0.08 |
| 989 | NZ_CP014508.1::WP_082779173.1 | Int115 | 0.05 |
| 990 | NZ_AP018205.1::WP_017291662.1 | Int116 | 0.27 |
| 995 | NZ_CP024313.1::WP_104825745.1 | Int117 | 0.09 |
| 1005 | NC_013856.1::WP_012977106.1 | Int118 | 0.44 |
| 1012 | NZ_CP032692.1::WP_120663868.1 | Int119 | 0.17 |
| 1015 | NZ_AP014686.1::WP_049810452.1 | Int120 | 0 |
| 1037 | NZ_CP032687.1::WP_120667728.1 | Int121 | 0.12 |
| 1042 | NZ_CP019604.1::WP_066842370.1 | Int122 | 0.43 |
| 1072 | NC_021908.1::WP_020920456.1 | Int123 | 0 |
| 1081 | NC_015597.1::WP_013851017.1 | Int124 | 0 |
| 1085 | NC_014825.1::WP_013483873.1 | Int125 | 0.06 |
| 1091 | NZ_CP018901.1::WP_057039620.1 | Int126 | 0.26 |
| 1095 | NZ_CP009453.1::WP_053556490.1 | Int127 | 0.03 |
| 1097 | NZ_CP023038.1::WP_010511773.1 | Int128 | 0.06 |
| 1101 | NC_014824.1::WP_013483611.1 | Int129 | 0 |
| 1102 | NZ_CP014527.1::WP_066137218.1 | Int130 | 0 |
| 1105 | NZ_CP017949.1::WP_106721837.1 | Int131 | 0.13 |
| 1106 | NZ_CP015292.1::WP_011331383.1 | Int132 | 0.48 |
| 1109 | NC_013164.1::WP_012797137.1 | Int133 | 0.34 |
| 1112 | NZ_CM008899.1::WP_058686676.1 | Int134 | 0.05 |
| 1113 | LN997845.1::CUW33404.1 | Int135 | 0 |
| 1114 | NZ_CP017949.1::WP_106721836.1 | Int136 | 0 |
| 1115 | NZ_CP011583.1::WP_031623921.1 | Int137 | 0 |
| 1116 | NZ_CP035407.1::WP_129137749.1 | Int138 | 27.56 |
| 1120 | NZ_CP046245.1::WP_156276654.1 | Int139 | 0 |
| 1124 | NZ_AP018296.1::WP_096695372.1 | Int140 | 0.07 |
| 1127 | NC_014825.1::WP_013483758.1 | Int141 | 0.1 |
| 1128 | NZ_CP040905.1::WP_139896886.1 | Int142 | 0.07 |
| 1131 | NZ_CP021678.1::WP_080626804.1 | Int143 | 0.03 |
| 1132 | NZ_CP013238.1::WP_045144988.1 | Int144 | 0.5 |
| 1135 | NC_004954.1::NP_862380.1 | Int145 | 0 |
| 1138 | NZ_CP020539.1::WP_081570537.1 | Int146 | 0.3 |
| 1140 | NZ_CP014289.1::WP_061885392.1 | Int147 | 0.34 |
| 1141 | NC_018688.1::WP_148283638.1 | Int148 | 0.64 |
| 1147 | NZ_CP032704.1::WP_145892019.1 | Int149 | 0 |
| 1153 | NZ_CP042515.1::WP_151523155.1 | Int150 | 0.29 |
| 1156 | NC_007411.1::WP_011316659.1 | Int151 | 0 |
| 1160 | NZ_AP014816.1::WP_066349550.1 | Int152 | 0 |
| 1163 | NZ_CP028186.1::WP_007215987.1 | Int153 | 0 |
| 1164 | NZ_CP016078.1::WP_075740684.1 | Int154 | 0 |
| 1165 | NC_018688.1::WP_000398825.1 | Int155 | 0.18 |
| 1167 | NZ_LR134433.1::WP_084758891.1 | Int156 | 0.1 |
| 1171 | NC_017791.1::WP_014686872.1 | Int157 | 0 |
| 1174 | NZ_LR594663.1::WP_162590241.1 | Int158 | 0 |
| 1175 | NZ_CP015456.1::WP_072285883.1 | Int159 | 0.11 |
| 1179 | NC_008704.1::WP_011562846.1 | Int160 | 0.07 |
| 1180 | NC_006907.1::YP_220381.1 | Int161 | 0.08 |
| 1182 | NZ_CP045481.1::WP_153030621.1 | Int162 | 0 |
| 1183 | NZ_CP014850.1::WP_033698958.1 | Int163 | 2.7 |
| 1188 | NC_018696.1::WP_085963899.1 | Int164 | 0.03 |
| 1189 | NZ_CP021746.1::WP_103654430.1 | Int165 | 0.32 |
| 1193 | NC_006362.1::WP_011212228.1 | Int166 | 0.07 |
| 1196 | NC_005016.1::NP_863503.1 | Int167 | 0 |
| 1198 | NZ_CP007723.1::WP_040145024.1 | Int168 | 0.14 |
| 129 | NC_041909.1::YP_009598586.1 | Int169 | 1.45 |
| 321 | NC_004820.1::NP_852555.1 | Int170 | 0.07 |
| 450 | FM864213.1::CAR95427.1 | Int171 | 0.16 |
| 541 | AY657002.1::AAT72399.1 | Int172 | 0.23 |
| 529 | KT336320.1::ALA07058.1 | Int173 | 0.35 |
| 34 | MF172979.1::ASD51140.1 | Int174 | 0.34 |
| 132 | MF172979.1::ASD51126.1 | Int175 | 0.16 |
| 322 | KP836356.2::AMS33992.1 | Int176 | 2.43 |
| 26 | MG711467.1::AUV56803.1 | Int177 | 0 |
| 152 | MG711466.1::AUV56714.1 | Int178 | 0.06 |
| 1226 | YP_001384783.1 | Int179 | 0 |
| 148 | KU160495.1::ALY08054.1 | Int180 | 3.49 |
| 9 | MF766046.1::ATI18835.1 | Int181 | 0.14 |
| 1227 | YP_001392519.1 | Int182 | 0.22 |
| 472 | NC_013646.1::YP_003347458.1 | Int183 | 0 |
| 51 | HQ906662.1::ADW80128.1 | Int184 | 0.05 |
| 33 | JQ680357.1::AFB75709.1 | Int185 | 1.01 |
| 1228 | BAF67264.1 | Int186 | 0 |
| 1229 | NP_470568.1 | Int187 | 0.05 |
| 52 | NC_005262.3::NP_944235.2 | Int188 | 4.42 |
| 36 | KY092483.1::APD18725.1 | Int189 | 0.06 |
| 134 | MH271320.1::AWY06686.1 | Int190 | 0.17 |
| 50 | MK450426.1::QAX94052.1 | Int191 | 11.46 |
| 165 | NC_013694.1::YP_003358736.1 | Int192 | 0.57 |
| 1230 | YP_706485.1 | Int193 | 0 |
| 97 | NC_021304.1::YP_008051452.1 | Int194 | 0.19 |
| 326 | NC_041983.1::YP_009607592.1 | Int195 | 0.1 |
| 420 | MH834619.1::AYN58532.1 | Int196 | 0.01 |
| 42 | MG593800.1::AUG87127.1 | Int197 | 0.66 |
| 29 | MK450433.1::QAX95039.1 | Int198 | 0.35 |
| 82 | MH248947.1::AWY07618.1 | Int199 | 0.17 |
| 516 | JX262376.1::AFO10918.1 | Int200 | 0.11 |
| 548 | NC_042051.1::YP_009616474.1 | Int201 | 0.11 |
| 333 | NC_041971.1::YP_009605116.1 | Int202 | 0.38 |
| 441 | MF140416.1::ASR87211.1 | Int203 | 0.19 |
| 354 | NC_028947.1::YP_009212783.1 | Int204 | 0.37 |
| 988 | NZ_CP005961.1::WP_042933187.1 | Int206 | 0.18 |
| 991 | NZ_CP030772.1::WP_138968811.1 | Int207 | 0.14 |
| 992 | NC_011987.1::WP_012653163.1 | Int208 | 0.17 |
| 993 | NZ_CP036427.1::WP_145267375.1 | Int209 | 0.06 |
| 994 | NZ_CP018231.1::WP_065283598.1 | Int210 | 0.31 |
| 996 | NZ_CP020899.1::WP_010009933.1 | Int211 | 0.12 |
| 997 | NZ_CP016290.1::WP_065284390.1 | Int212 | 0.15 |
| 998 | NZ_CP050090.1::WP_166481266.1 | Int213 | 0.27 |
| 999 | NZ_CP016619.1::WP_099510182.1 | Int214 | 0.1 |
| 1000 | NZ_AP014659.1::WP_035679705.1 | Int215 | 0.04 |
| 1001 | NC_020061.1::WP_004112891.1 | Int216 | 0.14 |
| 1002 | NZ_CP032692.1::WP_120764347.1 | Int217 | 0.1 |
| 1003 | NZ_CP016457.1::WP_069067140.1 | Int218 | 0 |
| 1004 | NZ_AP014687.1::WP_063824339.1 | Int219 | 0 |
| 1006 | NC_016588.1::WP_014189963.1 | Int220 | 0.09 |
| 1007 | NC_013860.1::WP_012978683.1 | Int221 | 0 |
| 1008 | NC_013857.1::WP_012977507.1 | Int222 | 0.17 |
| 1009 | NC_021909.1::WP_020923455.1 | Int223 | 0.14 |
| 1010 | NZ_CP018231.1::WP_072642081.1 | Int224 | 0 |
| 1011 | NZ_CP031599.1::WP_057822058.1 | Int225 | 0.15 |
| 1013 | NZ_CP020447.2::WP_080620360.1 | Int226 | 0 |
| 1014 | NZ_CP013053.1::WP_037377708.1 | Int227 | 0.17 |
| 1016 | NZ_CP012899.1::WP_157097479.1 | Int228 | 0 |
| 1017 | NZ_CP016289.1::WP_065283428.1 | Int229 | 0 |
| 1018 | NZ_CP018231.1::WP_065283441.1 | Int230 | 0.21 |
| 1019 | NZ_CP053209.2::WP_027688391.1 | Int231 | 0.16 |
| 1020 | NZ_CP025615.1::WP_102115455.1 | Int232 | 0.1 |
| 1021 | NZ_CP049159.1::WP_165098388.1 | Int233 | 0.01 |
| 1022 | NC_006824.1::WP_011254970.1 | Int234 | 0.14 |
| 1023 | NZ_HG916854.1::WP_051509115.1 | Int235 | 0.05 |
| 1024 | NC_010627.1::WP_012404129.1 | Int236 | 0.24 |
| 1025 | NZ_CP023072.1::WP_037435892.1 | Int237 | 0.05 |
| 1026 | NC_000914.2::WP_010875070.1 | Int238 | 0 |
| 1027 | NZ_CP021815.1::WP_088198182.1 | Int239 | 0 |
| 1028 | NZ_CP026529.1::WP_088199679.1 | Int240 | 0.02 |
| 1029 | NC_019847.2::WP_015241694.1 | Int241 | 0 |
| 1030 | NC_019847.2::WP_049589666.1 | Int242 | 0.04 |
| 1031 | NZ_CP013419.1::WP_059581534.1 | Int243 | 0 |
| 1032 | NC_013193.1::WP_012806738.1 | Int244 | 0.12 |
| 1033 | NZ_CP013419.1::WP_059669918.1 | Int245 | 0.11 |
| 1034 | NZ_CP026685.1::WP_104902331.1 | Int246 | 0.18 |
| 1035 | NZ_CP024793.1::WP_100897719.1 | Int247 | 0.11 |
| 1036 | NZ_CP049701.1::WP_166349160.1 | Int248 | 0.22 |
| 1038 | NZ_CP023072.1::WP_037435909.1 | Int249 | 0 |
| 1039 | NZ_CP021216.1::WP_014531100.1 | Int250 | 0 |
| 1040 | NZ_CP014311.1::WP_062175057.1 | Int251 | 0 |
| 1041 | NC_009468.1::WP_043508908.1 | Int252 | 0.05 |
| 1043 | NZ_CP038639.1::WP_135707565.1 | Int253 | 0 |
| 1044 | NZ_CP017077.1::WP_069709769.1 | Int254 | 0.06 |
| 1045 | NZ_CP016620.1::WP_099515887.1 | Int255 | 0.28 |
| 1046 | NZ_CP016619.1::WP_099513340.1 | Int256 | 0.06 |
| 1047 | NC_008308.1::YP_718035.1 | Int257 | 0.03 |
| 1048 | NZ_CP015322.1::WP_006199992.1 | Int258 | 0.33 |
| 1049 | NZ_CP026528.1::WP_158528806.1 | Int259 | 0.07 |
| 1050 | NZ_LR594691.1::WP_102905083.1 | Int260 | 0 |
| 1051 | NC_020562.1::WP_015460498.1 | Int261 | 0 |
| 1052 | NZ_CP044544.1::WP_100951630.1 | Int262 | 0.22 |
| 1053 | NC_008760.1::WP_011798428.1 | Int263 | 0.05 |
| 1054 | NZ_CP013544.1::WP_011053437.1 | Int264 | 0 |
| 1055 | NZ_CP013572.1::WP_081278377.1 | Int265 | 0 |
| 1056 | NZ_CP018231.1::WP_081374274.1 | Int266 | 0.32 |
| 1057 | NZ_CP024313.1::WP_104825738.1 | Int267 | 0.44 |
| 1058 | NC_008378.1::WP_011649403.1 | Int268 | 0.03 |
| 1059 | NZ_CP017243.1::WP_004675975.1 | Int269 | 0.17 |
| 1060 | NZ_CP021214.1::WP_017273893.1 | Int270 | 0.1 |
| 1061 | NZ_CP023072.1::WP_095689837.1 | Int271 | 0.45 |
| 1062 | NC_009508.1::WP_011950828.1 | Int272 | 0.06 |
| 1063 | NZ_CP013535.1::WP_081279173.1 | Int273 | 0.01 |
| 1064 | NZ_CP017077.1::WP_069709873.1 | Int274 | 0.11 |
| 1065 | NZ_CP017077.1::WP_083274844.1 | Int275 | 0.4 |
| 1066 | NZ_CP016619.1::WP_099513354.1 | Int276 | 0 |
| 1067 | NZ_CP030355.1::WP_082057937.1 | Int277 | 0.19 |
| 1068 | NZ_CP015745.1::WP_064334697.1 | Int278 | 0.1 |
| 1069 | NZ_CP034911.1::WP_069456400.1 | Int279 | 0.26 |
| 1070 | NZ_CP030764.1::WP_112905663.1 | Int280 | 0 |
| 1071 | NC_007762.1::WP_011427411.1 | Int281 | 0.11 |
| 1073 | NZ_CP013633.1::WP_064842796.1 | Int282 | 0 |
| 1074 | NC_004041.2::WP_011053488.1 | Int283 | 0.5 |
| 1075 | NC_008381.1::WP_011654186.1 | Int284 | 0.14 |
| 1076 | NZ_CP020910.1::WP_086083774.1 | Int285 | 0 |
| 1077 | NZ_CP025015.1::WP_105009893.1 | Int286 | 0.17 |
| 1078 | NC_015579.1::WP_013831319.1 | Int287 | 0.06 |
| 1079 | NZ_CP039651.1::WP_109154411.1 | Int288 | 0.11 |
| 1080 | NZ_CP030762.1::WP_112907845.1 | Int289 | 0 |
| 1082 | NZ_CP016289.1::WP_065284176.1 | Int290 | 0.13 |
| 1083 | NZ_CP021819.1::WP_088201210.1 | Int291 | 0.14 |
| 1084 | NC_007960.1::WP_041359701.1 | Int292 | 0.04 |
| 1086 | NZ_CP026528.1::WP_158528808.1 | Int293 | 0.14 |
| 1087 | NZ_AP014686.1::WP_080587274.1 | Int294 | 0.11 |
| 1088 | NC_020061.1::WP_004112912.1 | Int295 | 0.07 |
| 1089 | NZ_CP017563.1::WP_154671697.1 | Int296 | 0.26 |
| 1090 | NZ_CP049735.1::WP_165586638.1 | Int297 | 0.2 |
| 1099 | NZ_CP016620.1::WP_099515711.1 | Int302 | 0 |
| 1100 | NZ_CP023549.1::WP_096787955.1 | Int303 | 0 |
| 1103 | NZ_CP014527.1::WP_066137015.1 | Int304 | 0 |
| 1104 | NZ_CP035512.1::WP_082731421.1 | Int305 | 0 |
| 1107 | NZ_CP021071.1::WP_084015878.1 | Int306 | 0 |
| 1108 | NZ_CP016453.1::WP_083217015.1 | Int307 | 0 |
| 1110 | NZ_CP016620.1::WP_099515874.1 | Int308 | 0 |
| 1111 | NZ_CP049701.1::WP_166354437.1 | Int309 | 0 |
| 1117 | NC_014825.1::WP_013483791.1 | Int310 | 0 |
| 1118 | NC_014825.1::WP_013483878.1 | Int311 | 0 |
| 1119 | NZ_CP044332.1::WP_016919146.1 | Int312 | 0 |
| 1121 | NZ_CP033248.1::WP_153738853.1 | Int313 | 0 |
| 1122 | NZ_CP013238.1::WP_071981582.1 | Int314 | 0 |
| 1123 | NZ_CM003332.1::WP_039285843.1 | Int315 | 0 |
| 1125 | NZ_CP015585.1::WP_083671481.1 | Int316 | 0 |
| 1126 | NZ_CP018336.1::WP_073541495.1 | Int317 | 0.55 |
| 1129 | NZ_CP033248.1::WP_153738854.1 | Int318 | 0 |
| 1130 | NC_014825.1::WP_013483874.1 | Int319 | 0 |
| 1133 | NZ_CP007453.1::WP_025436591.1 | Int320 | 0 |
| 1134 | NZ_CP017949.1::WP_106721914.1 | Int321 | 0 |
| 1137 | NZ_CP008945.1::WP_038606886.1 | Int322 | 0.17 |
| 1139 | NZ_CP046254.1::WP_140043508.1 | Int323 | 0 |
| 1142 | NC_002682.1::WP_044547337.1 | Int324 | 0.04 |
| 1143 | NC_008826.1::WP_011828792.1 | Int325 | 0.11 |
| 1144 | NZ_AP022320.1::WP_162071183.1 | Int326 | 0 |
| 1145 | NZ_LR135387.1::WP_000136908.1 | Int327 | 0 |
| 1146 | CP033508.1::QKC67623.1 | Int328 | 0 |
| 1148 | NZ_CP016080.1::WP_027047857.1 | Int329 | 0 |
| 1149 | NZ_CP032928.1::WP_162993116.1 | Int330 | 0 |
| 1150 | NZ_LR135483.1::WP_033652754.1 | Int331 | 0.09 |
| 1151 | NZ_LR594668.1::WP_162571537.1 | Int332 | 0 |
| 1152 | NZ_CP014289.1::WP_061885389.1 | Int333 | 0 |
| 1154 | NC_014825.1::WP_013483880.1 | Int334 | 0 |
| 1155 | NZ_CP014308.1::WP_062174163.1 | Int335 | 0 |
| 1157 | NZ_CP033248.1::WP_153738977.1 | Int336 | 0 |
| 1158 | NZ_LN907829.1::WP_067437236.1 | Int337 | 0 |
| 1159 | NZ_CM017044.1::WP_000709098.1 | Int338 | 0 |
| 1161 | NC_014825.1::WP_013483789.1 | Int339 | 0 |
| 1166 | NZ_CP031591.1::WP_111772986.1 | Int340 | 0 |
| 1168 | NC_003064.2::WP_162180340.1 | Int341 | 0 |
| 1169 | NZ_AFSD01000008.1::WP_035243797.1 | Int342 | 0 |
| 1170 | NZ_CP039905.1::WP_080843366.1 | Int343 | 0 |
| 1172 | NZ_LR594663.1::WP_162590298.1 | Int344 | 0 |
| 1173 | NC_009717.1::WP_157048325.1 | Int345 | 0 |
| 1176 | NZ_CP016593.1::WP_014538220.1 | Int346 | 0 |
| 1177 | NC_019761.1::WP_015211582.1 | Int347 | 0 |
| 1178 | NZ_CP014289.1::WP_061885391.1 | Int348 | 0 |
| 1181 | NC_007961.1::WP_011505253.1 | Int349 | 0 |
| 1184 | NZ_CP015439.1::WP_066328033.1 | Int350 | 0 |
| 1185 | AP022559.1::BBW99057.1 | Int351 | 0.52 |
| 1186 | NZ_LR134446.1::WP_068741343.1 | Int352 | 0.03 |
| 1187 | NC_019957.1::WP_015297851.1 | Int353 | 0 |
| 1190 | NZ_CP007453.1::WP_025436590.1 | Int354 | 0 |
| 1191 | NZ_CP017949.1::WP_106721912.1 | Int355 | 0 |
| 1195 | NZ_CP008945.1::WP_038606889.1 | Int356 | 0 |
| 507 | KY963369.1::ARW58402.1 | Int357 | 0 |
| 504 | KT336320.1::ALA07059.1 | Int358 | 0 |
| 530 | KT336321.1::ALA07121.1 | Int359 | 0 |
| 505 | KT336321.1::ALA07122.1 | Int360 | 0 |
| 531 | KX077896.1::ANM47644.1 | Int361 | 0 |
| 506 | KX077896.1::ANM47643.1 | Int362 | 0 |
| 532 | FN997652.1::CBR26922.1 | Int363 | 0 |
| 451 | FN997652.1::CBR26923.1 | Int364 | 0 |
| 133 | MF172979.1::ASD51128.1 | Int365 | 0 |
| 160 | MK448666.1::QBX13692.1 | Int366 | 0 |
| 552 | MK448666.1::QBX13693.1 | Int367 | 0 |
| 121 | MK448667.1::QBX13733.1 | Int368 | 0 |
| 106 | MK448667.1::QBX13731.1 | Int369 | 0 |
| 533 | MK448674.1::QBX14111.1 | Int370 | 0 |
| 508 | MK448674.1::QBX14110.1 | Int371 | 0 |
| 122 | MK448687.1::QBX14891.1 | Int372 | 0 |
| 542 | MK448687.1::QBX14890.1 | Int373 | 0 |
| 154 | MK448700.1::QBX15583.1 | Int374 | 0 |
| 107 | MK448700.1::QBX15585.1 | Int375 | 0 |
| 543 | MK448713.1::QBX16269.1 | Int376 | 0 |
| 550 | MK448719.1::QBX16517.1 | Int377 | 0 |
| 123 | MK448719.1::QBX16516.1 | Int378 | 0 |
| 144 | MK448720.1::QBX16591.1 | Int379 | 0 |
| 509 | MK448741.1::QBX17590.1 | Int380 | 0 |
| 534 | MK448741.1::QBX17591.1 | Int381 | 0 |
| 535 | MK448752.1::QBX18231.1 | Int382 | 0 |
| 510 | MK448752.1::QBX18232.1 | Int383 | 0 |
| 536 | MK448811.1::QBX21447.1 | Int384 | 0 |
| 124 | MK448819.1::QBX21895.1 | Int385 | 0 |
| 553 | MK448819.1::QBX21894.1 | Int386 | 0 |
| 125 | MK448825.1::QBX22171.1 | Int387 | 0 |
| 554 | MK448825.1::QBX22172.1 | Int388 | 0 |
| 126 | MK448835.1::QBX22708.1 | Int389 | 0 |
| 127 | MK448836.1::QBX22750.1 | Int390 | 0 |
| 518 | MK448838.1::QBX22844.1 | Int391 | 0 |
| 547 | MK448838.1::QBX22843.1 | Int392 | 0 |
| 135 | MK448846.1::QBX23238.1 | Int393 | 0.22 |
| 545 | MK448846.1::QBX23239.1 | Int394 | 0 |
| 546 | MK448847.1::QBX23319.1 | Int395 | 0 |
| 136 | MK448847.1::QBX23320.1 | Int396 | 0 |
| 155 | MK448934.1::QBX27918.1 | Int397 | 0 |
| 537 | MK448935.1::QBX27959.1 | Int398 | 0 |
| 519 | MK448935.1::QBX27960.1 | Int399 | 0 |
| 512 | MK448994.1::QBX31153.1 | Int400 | 0 |
| 538 | MK448994.1::QBX31154.1 | Int401 | 0 |
| 137 | MK448997.1::QBX31307.1 | Int402 | 0.1 |
| 138 | MK448997.1::QBX31309.1 | Int403 | 0 |
| 539 | MK448999.1::QBX31462.1 | Int404 | 0 |
| 540 | MK359990.1::QEM40854.1 | Int405 | 0 |
| 454 | MK359990.1::QEM40855.1 | Int406 | 0.19 |
| 1231 | YP_002336631.1 | Int407 | 0 |
| 1232 | YP_001646422.1 | Int408 | 0 |
| 183 | JQ660954.1::AFJ96082.1 | Int409 | 13.57 |
| 72 | NC_021865.1::YP_008320369.1 | Int410 | 1.65 |
| 184 | NC_030950.1::YP_009277275.1 | Int411 | 0 |
| 163 | NC_018264.1::YP_006546326.1 | Int412 | 0 |
| 185 | KM983327.1::AJA42491.1 | Int413 | 0 |
| 11 | MF766047.1::ATI18915.1 | Int414 | 0 |
| 10 | MF766048.1::ATI18993.1 | Int415 | 0 |
| 7 | MH590601.1::AXH70257.1 | Int416 | 0 |
| 17 | MH669004.1::AXQ61107.1 | Int417 | 0 |
| 8 | MK392364.1::QAY15794.1 | Int418 | 0 |
| 68 | KU517658.1::AMB17413.1 | Int419 | 0 |
| 1233 | NP_268897.1 | Int420 | 0 |
| 1234 | YP_005869510.1 | Int421 | 0 |
| 1235 | YP_002736920.1 | Int422 | 0.17 |
| 1236 | YP_003445547.1 | Int423 | 0.35 |
| 1237 | NP_112664.1 | Int424 | 0.33 |
| 1238 | YP_002747001.1 | Int425 | 0 |
| 480 | NC_027982.1::YP_009167795.1 | Int426 | 0 |
| 350 | NC_031929.1::YP_009323520.1 | Int427 | 5.2 |
| 157 | KX456210.1::ANS02547.1 | Int428 | 0 |
| 158 | DQ394810.1::ABD63849.1 | Int429 | 0.26 |
| 374 | KY349816.1::APZ81892.1 | Int430 | 0 |
| 345 | KJ608189.1::AIS74015.1 | Int431 | 12.97 |
| 372 | MF417874.1::ASN68226.1 | Int432 | 1.69 |
| 366 | MK448669.1::QBX13835.1 | Int433 | 0 |
| 410 | MK448672.1::QBX14038.1 | Int434 | 0 |
| 339 | MK448705.1::QBX15858.1 | Int435 | 0.1 |
| 340 | MK448708.1::QBX15966.1 | Int436 | 0 |
| 377 | MK448714.1::QBX16272.1 | Int437 | 0 |
| 341 | MK448742.1::QBX17688.1 | Int438 | 0 |
| 411 | MK448778.1::QBX19706.1 | Int439 | 0 |
| 404 | MK448826.1::QBX22213.1 | Int440 | 0 |
| 378 | MK448831.1::QBX22445.1 | Int441 | 0 |
| 342 | MK448834.1::QBX22610.1 | Int442 | 0 |
| 405 | MK448844.1::QBX23130.1 | Int443 | 0 |
| 412 | MK448849.1::QBX23375.1 | Int444 | 0 |
| 343 | MK448873.1::QBX24735.1 | Int445 | 0 |
| 381 | MK448874.1::QBX24736.1 | Int446 | 0 |
| 406 | MK448875.1::QBX24786.1 | Int447 | 0.16 |
| 346 | MK448878.1::QBX24961.1 | Int448 | 0 |
| 367 | MK448879.1::QBX25020.1 | Int449 | 0 |
| 407 | MK448898.1::QBX26003.1 | Int450 | 0 |
| 382 | MK448927.1::QBX27562.1 | Int451 | 0 |
| 344 | MK448940.1::QBX28214.1 | Int452 | 0 |
| 383 | MK448986.1::QBX30733.1 | Int453 | 0 |
| 421 | MK449012.1::QBX32092.1 | Int454 | 0 |
| 38 | MG711465.1::AUV56620.1 | Int455 | 0 |
| 49 | MF417875.1::ASN68324.1 | Int456 | 6.77 |
| 1239 | BAE05705.1 | Int457 | 0 |
| 1240 | YP_003472505.1 | Int458 | 0 |
| 1241 | BAF92844.1 | Int459 | 3.29 |
| 456 | NC_007064.1::YP_240778.1 | Int460 | 0 |
| 457 | NC_007065.1::YP_240852.1 | Int461 | 15.17 |
| 464 | NC_008722.1::YP_950630.1 | Int462 | 0 |
| 465 | NC_008723.1::YP_950693.1 | Int463 | 0 |
| 466 | NC_031241.1::YP_009302049.1 | Int464 | 0 |
| 459 | NC_020490.2::YP_009130680.1 | Int465 | 0 |
| 500 | NC_019915.1::YP_007236622.1 | Int466 | 0 |
| 438 | NC_024391.1::YP_009044994.1 | Int467 | 0 |
| 467 | MF417895.1::ASN69744.1 | Int468 | 0 |
| 468 | MF417901.1::ASN70113.1 | Int469 | 0 |
| 469 | MF417930.1::ASN71670.1 | Int470 | 0 |
| 470 | MF417982.1::ASN72884.1 | Int471 | 0 |
| 423 | MF417958.1::ASN72539.1 | Int472 | 0 |
| 1242 | YP_003251752.1 | Int473 | 0 |
| 370 | NC_019544.1::YP_007010946.1 | Int474 | 0 |
| 437 | DQ453159.1::ABI36844.1 | Int475 | 2.64 |
| 392 | HM072038.1::ADF59162.1 | Int476 | 3.07 |
| 433 | KX965989.1::APC46450.1 | Int477 | 0 |
| 444 | MF417925.1::ASN71428.1 | Int478 | 11.77 |
| 445 | MF417893.1::ASN69614.1 | Int479 | 6.84 |
| 393 | MF417886.1::ASN69149.1 | Int480 | 38.07 |
| 379 | MK560763.1::QBP06974.1 | Int481 | 0.36 |
| 37 | KY092479.1::APD18506.1 | Int482 | 0 |
| 41 | KY092482.1::APD18671.1 | Int483 | 0 |
| 39 | KY092481.1::APD18613.1 | Int484 | 0 |
| 57 | KY092480.1::APD18560.1 | Int485 | 0.21 |
| 44 | KY092484.1::APD18778.1 | Int486 | 0.09 |
| 45 | MH825699.1::AYD86220.1 | Int487 | 0.72 |
| 143 | MK450431.1::QAX94753.1 | Int488 | 0 |
| 145 | JQ809701.1::AFL47903.1 | Int489 | 0 |

Individual polypeptides and cognate recognition sequences are shown in Table 22 with their Integrase A No. in column 1 (with “Integrase A No.” corresponding to the respective Integrase No. in the sequence listing attached herewith) and were assigned an integrase identification name (“IntID”) in column 3. The integration efficiency is indicated in column 4 as the percent of cells expressing GFP (“GFP+”) as measured by flow cytometry at 21 days post-transfection in the absence of antibiotic selection.

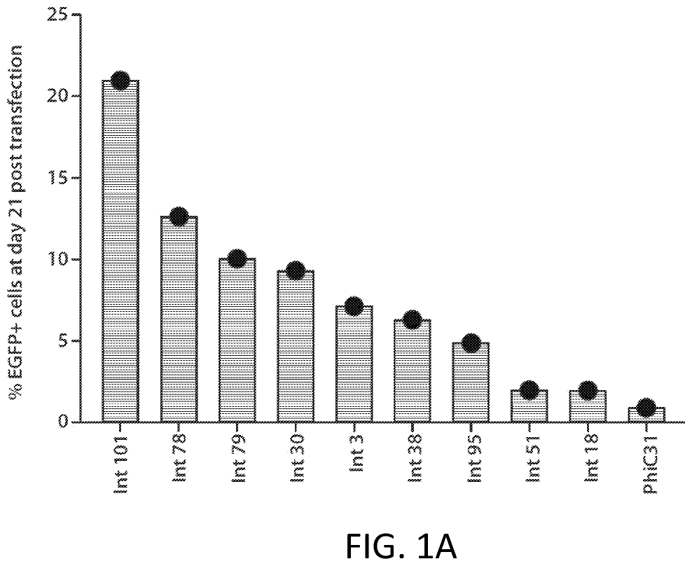

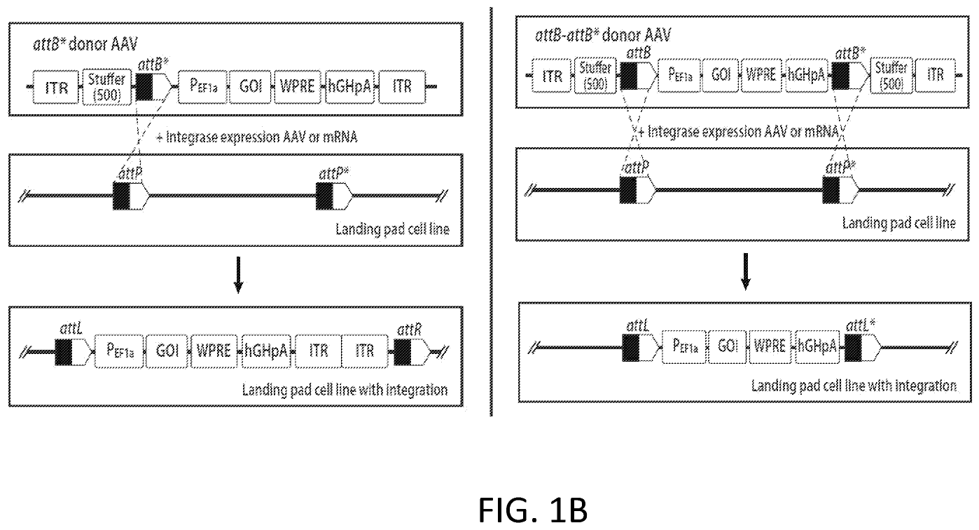

In a further example, BTEK293T cells were transfected with an integrase expression plasmid and a template plasmid harboring a 520 bp attP containing region followed by an EGFP reporter driven by CMV promoter. The percentage of EGFP positive cells at day 21 post-transfection was analyzed by flow cytometry. As shown in FIG. 1A, 9 out of 9 integrases depicted achieved higher integration efficiency compared to the positive control integrase PhiC31 in 293T cells. Data for integrases shown comprised greater than 2 replicates.

### Example 14: Dual AAV Delivery of Serine Integrase and Template DNA to Mammalian Cells

This example demonstrates the use of a serine recombinase based Gene Writer system for the targeted integration of a template DNA into the human genome. More specifically, a recombinase, e.g., an integrase comprising an amino acid sequence of any of SEQ ID NOs: 1-12,677 (e.g., SEQ ID NOs: 1-11,432), e.g., the Bxb1 recombinase protein (e.g., comprising an amino acid sequence of SEQ ID NO: 11,636), and a template DNA comprising the associated attachment site, e.g., a sequence from a LeftRegion or RightRegion, e.g., comprising a sequence of any of SEQ ID NOs: 13,001-25,677 or SEQ ID NOs: 26,001-38,677, respectively (e.g., SEQ ID NOs: 24,636 or 37,636, respectively), are co-delivered to HEK293T cells as separate AAV viral vectors to insert DNA precisely and efficiently in a mammalian cell genome containing the corresponding Bxb1 attachment landing pad site.

Two transgene configurations are assessed to determine the integration, stability, and expression using different AAV donor formats (FIG. 1B): 1) template comprising attP* or attB* that utilizes formation of double-stranded circularized DNA following AAV transduction in the cell nucleus; or 2) template comprising double attachment sites, attP-attP* or attB-attB*, that can integrate into the mammalian genome independent of double-stranded circularization of the DNA following AAV transduction in the cell nucleus.

To prepare HEK293T cells for Bxb1-mediated genomic integration of a template, HEK293T landing pad cell lines were generated containing the Bxb1 attP-attP* or Bxb1 attB-attB* sites. HEK293T cells were seeded in 10 cm plates (5×10⁶ cells) prior to lentiviral transfection. Lentiviral transduction using the Lenti-X Packaging Single Shots (VSV-G, Takara Bio) was performed the following day with lentiviral vector plasmid DNA (containing attP-attP* or attB-attB*). Lentiviral titering was performed and the virus filtered using 0.22 m filter and 1 mL lentiviral aliquots were made and stored at −80° C. HEK293T cells were seeded at 1×10⁵ cells/well in 4×6-well plates. HEK293T cells were then transduced with attP-attP* or attB-attB* lentivirus and cultured for 48 hours before starting puromycin selection (1 g/mL). Cells were kept under puromycin selection for at least 7 days and then scaled up to 150 mm culture plates. The cells were then harvested for genomic DNA (gDNA) and assayed for lentivirus integration copy number by ddPCR.

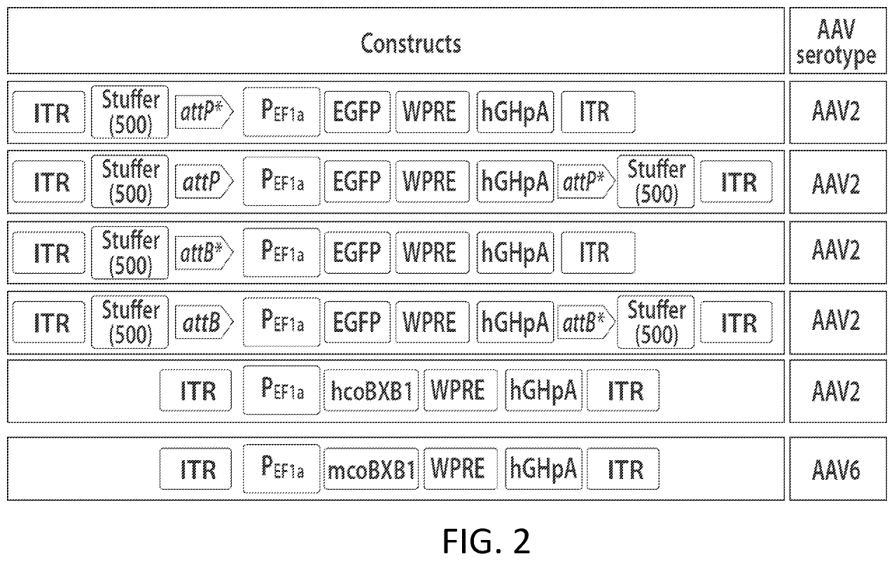

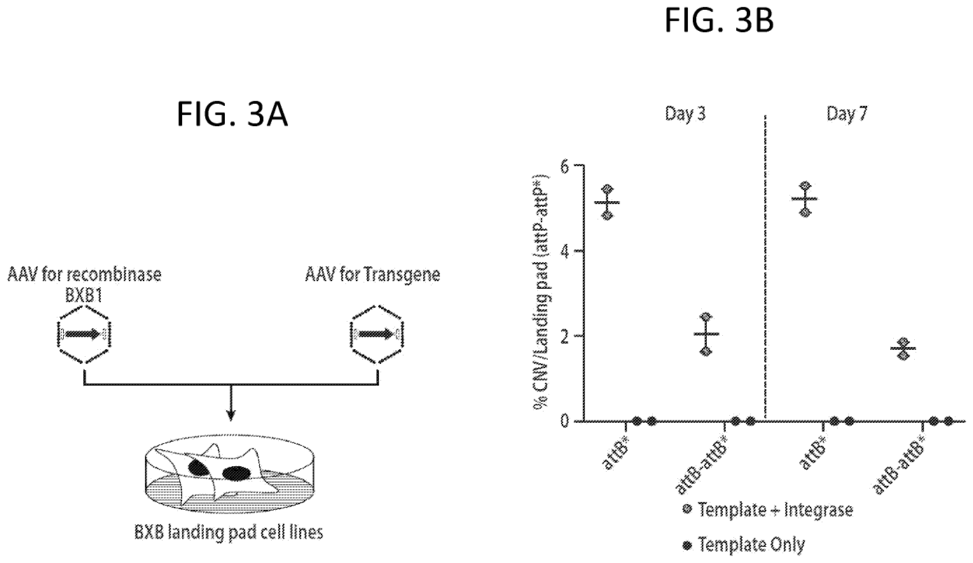

Adeno-associated viral vectors containing Bxb1 integrase or the corresponding Bxb1 attP*/attP-attP* donor or Bxb1 attB*/attB-attB* donor were generated based on the pAAV-CMV-EGFP-WPRE-pA viral backbone (Sirion Biotech), but with replacement of the CMV promoter with the EF1a promoter. pAAV-Ef1a-BXB1-WPRE-pA was generated using a human codon optimized Bxb1 (GenScript). pAAV-Stuffer-attP*(Bxb1)-Ef1a-EGFP-WPRE-pA and pAAV-Stuffer-attB*(Bxb1)-Ef1a-EGFP-WPRE-pA template constructs contained a 500 bp stuffer sequence between the 5′ AAV2 ITR sequence and Ef1a promoter. pAAV-Stuffer-attP(Bxb1)-Ef1a-EGFP-WPRE-pA-attP*(Bxb1)-Stuffer and pAAV-Stuffer-attB(Bxb1)-Ef1a-EGFP-WPRE-pA-attB*(Bxb1)-Stuffer donor constructs contained a 500 bp stuffer sequence between the AAV2 ITR sequence and Ef1a promoter, as well as a 500 bp stuffer sequence between the 3′ attP*/attB* attachment site and 3′ AAV2 ITR sequence (FIG. 2). The above listed AAV vectors were packaged into AAV2 serotype (Sirion Biotech) at a 113 total vg scale: AAV2-Ef1a-BXB]-WPRE-pA, AAV2-Stuffer-attP*(BXB1)-Ef1a-EGFP-WPRE-pA, AAV2-Stuffer-attB*(BXB1)-Ef1a-EGFP-WPRE pA, AAV2-Stuffer-attP(BXB1)-Ef1a-EGFP-WPRE-pA-attP*(BXB1)-Stuffer, AAV2-Stuffer-attB(BXBI)-Ef1a-EGFP-WPRE-pA-attB*(BXB1)-Stuffer. HEK293T landing pad cells containing either attP-attP* or attB-attB* landing pad sites were seeded in a 48-well plate format at 40,000 cells/well. 24 h later, the following conditions were tested: dual AAV transduction with 1) AAV2-attP*-Ef1a-EGFP with or without AAV2-Ef1a-BXB1 integrase, 2) AAV2-attP-attP*-Ef1a-EGFP donor with or without AAV2-Ef1a-BXB1 integrase, 3) AAV2-attB*-Ef1a-EGFP with or without AAV2-Ef1a-BXB1 integrase, 4) AAV2-attB-attB*-Ef1a-EGFP with or without AAV2-Ef1a-BXB1 integrase (FIG. 3A). The AAV comprising the integrase was dosed at an MOI of about 25,000, and the AAV comprising the template was dosed at an MOI of about 75,000. To assess the efficiency of a dual AAV delivery of a serine integrase and a template comprising its recognition site to integrate into the human genome, ddPCR was performed to quantify integration events (% CNV/landing pad) on day 3 and day 7 post-transduction. ˜5% integration was detected using an attB* donor to attP-attP* landing pad cell line, and this integration was stable and consistent at both timepoints (FIG. 3B), indicative of successful DNA Gene Writing by a dual AAV delivery system.

### Example 15: In Vitro Combination mRNA and AAV Delivery of a Gene Writing Polypeptide and Template DNA for Site-Specific Integration in Human Cells

This example demonstrates use of a Gene Writer system for the site-specific insertion of exogenous DNA into the mammalian cell genome. More specifically, a recombinase, e.g., an integrase comprising an amino acid sequence of any of SEQ ID NOs: 1-12,677 (e.g., SEQ ID NOs: 1-11,432), e.g., the Bxb1 recombinase protein (e.g., comprising an amino acid sequence of SEQ ID NO: 11,636, and a template DNA comprising the associated attachment site, e.g., a sequence from a LeftRegion or RightRegion, a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively, e.g., the LeftRegion comprising a sequence of SEQ ID NO: 24,636, are introduced into a HEK293T landing pad cell line. In this example, the recombinase is delivered as mRNA encoding the recombinase, and the template DNA is delivered via AAV.

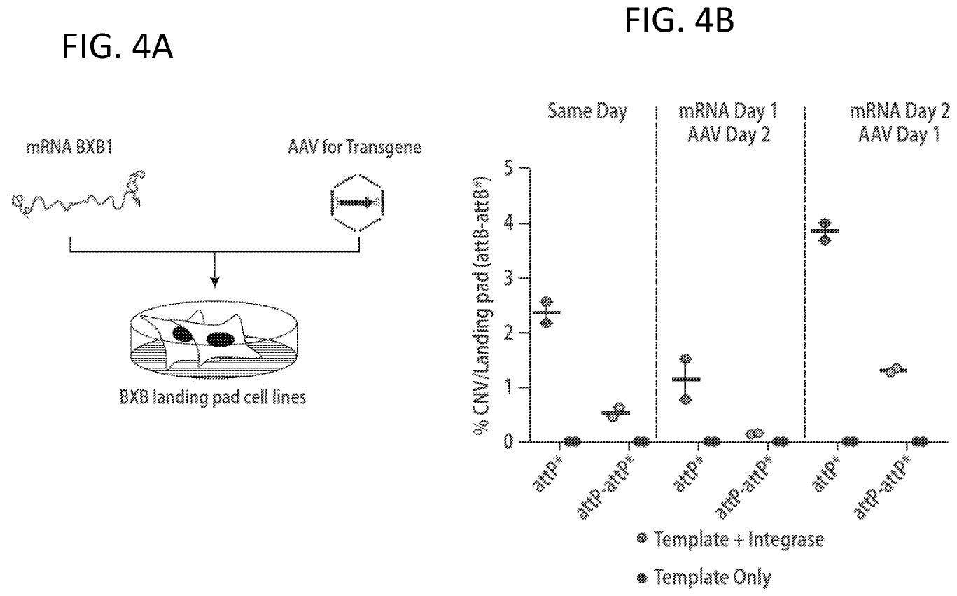

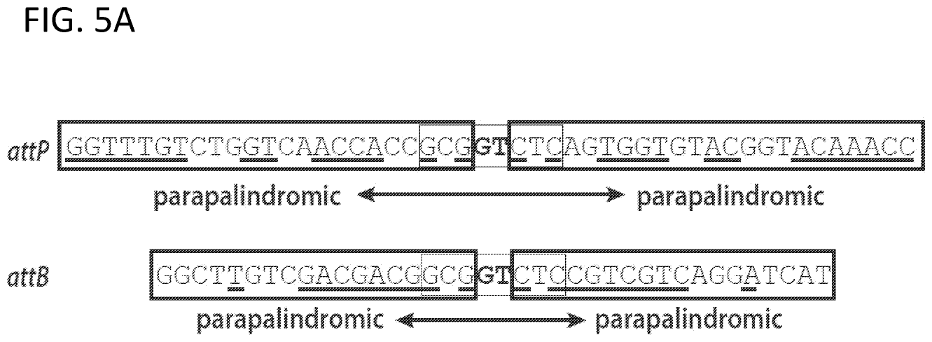

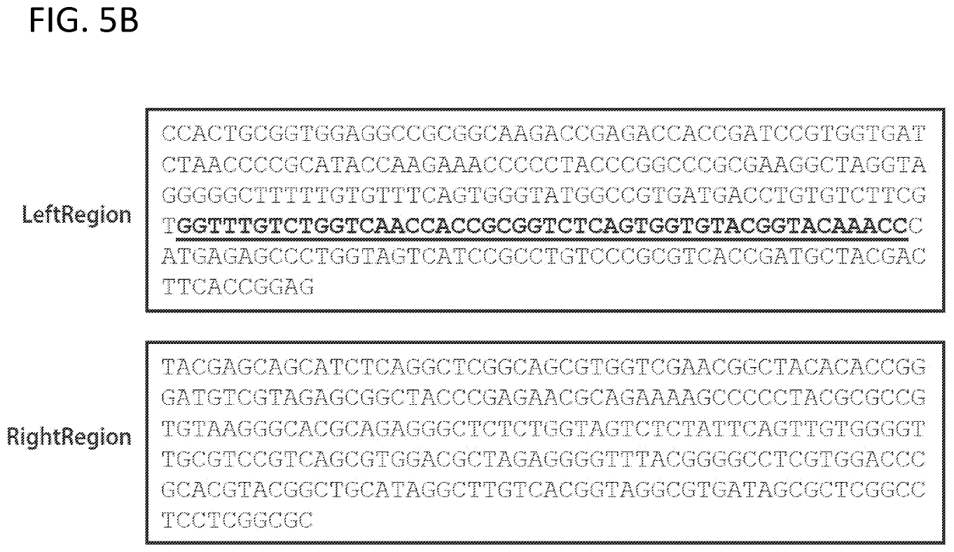

HEK293T landing pad cells containing either the attP-attP* or attB-attB* landing pad sites (see Example 14) were seeded in a 48-well plate format at 40,000 cells/well. 24 h later, the following conditions were tested: 1) AAV2-attP*-Ef1a-EGFP with or without mRNA encoding the BXB1 integrase; 2) AAV2-attP-attP*-Ef1a-EGFP donor with or without mRNA encoding the BXB1 integrase; 3) AAV2-attB*-Ef1a-EGFP with or without mRNA encoding the BXB1 integrase; and 4) AAV2-attB-attB*-Ef1a-EGFP with or without mRNA encoding the BXB1 integrase (FIG. 4A). The mRNA encoding the integrase was dosed at about 1 g and the AAV comprising the template was dosed at an MOI of about 75,000. The timing of delivery was also assessed by the following conditions: 1) mRNA delivery of BXB1 integrase and AAV delivery of template DNA on the same day, 2) mRNA delivery of BXB1 integrase 24 h prior to AAV delivery of template DNA, 3) AAV delivery of template DNA 24 h prior to mRNA delivery of BXB1 integrase. ddPCR was performed to assess the integration mediated through mRNA delivery of a serine integrase and AAV delivery of a template comprising its attachment, ddPCR was performed to assay for integration (% CNV/landing pad) on day 3 post-transfection of mRNA and post-transduction of AAV. ˜24% integration was detected using an attP* donor to attB-attB* landing pad 293T cell line (FIG. 4B). AAV delivery of attachment site donor 24 h prior to mRNA delivery of BXB1 integrase achieved the highest % CNV/landing pad of ˜4% (FIG. 3B). These results are indicative of successful DNA Gene Writing genome editing events that insert the AAV-delivered DNA fragment that is site-specific, mediated by mRNA delivery of serine integrase and AAV delivery of its respective site-specific attachment site.

### Example 16: Ex Vivo Combination mRNA and AAV Delivery of a Gene Writing Polypeptide and Template DNA to HSCs for the Treatment of Beta-Thalassemia and Sickle Cell Disease

This example describes delivery of mRNA encoding an integrase and AAV template DNA into C34+ cells (hematopoietic stem and progenitor cells) in order to write an actively expressed 7-globin gene cassette to treat genetic mutations that lead to beta-thalassemia and sickle cell disease.

In this example, AAV6 is used to deliver the template DNA. More specifically, the AAV6 template DNA includes, in order, 5′ ITR, an integrase attachment site, e.g., an attP or attB, e.g., a LeftRegion or RightRegion comprising a nucleic acid sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively, a pol II promoter, e.g., the human β-globin promoter, a human fetal 7-globin coding sequence, a poly A tail and 3′ITR. Considering the maximum volume limit of electroporation reagents, integrase mRNA and the AAV6 template are co-delivered into CD34 cells via different conditions, e.g.: 1) AAV6 template and integrase mRNA are co-electroporated; 2) integrase mRNA is electroporated 15 mins prior to AAV6 donor transduction.

After electroporation/transduction, cells are incubated in CD34 maintenance media for 2 days. Then, ˜10% of the treated cells are harvested for genomic DNA isolation to determine integration efficiency. The rest of the cells are transferred to erythroid expansion and differentiation media. After ˜20 days differentiation, three assays will be performed to determine the integration of 7-globin after erythroid differentiation: 1) a subset of cells is stained with NucRed (Thermo Fisher Scientific) to determine the enucleation rate; 2) a subset of the cells is stained with fluorescein isothiocyanate (FITC)-conjugated anti-7-globin antibody (Santa Cruz) to determine the percentage of fetal hemoglobin positive cells; 3) a subset of the cells is harvested for HPLC to determine γ-globin chain expression.

### Example 17: Ex Vivo Delivery of a Gene Writer Polypeptide and Circular DNA Template for Generating CAR-T Cells

In this example, a Gene Writing system is delivered as a deoxyribonucleoprotein (DNP) to human primary T-cells ex vivo for the generation of CAR-T cells, e.g., CAR-T cells for treating B-cell lymphoma.

The Gene Writer polypeptide, e.g., integrase, e.g., integrase comprising an amino acid sequence of any of SEQ ID NOs: 1-12,677 (e.g., SEQ ID NOs: 1-11,432), is prepared and purified for use directly in its active protein form. For the template component, minicircle DNA plasmids that lack plasmid backbone and bacterial sequences are used in this example, e.g., prepared as according to a method of Chen et al. Mol Ther 8(3):495-500 (2003), wherein a recombination event is first used to excise these extraneous plasmid maintenance functions to minimize plasmid size and cellular response. Template DNA minicircles comprise, in order, an integrase attachment site (attP or attB), e.g., a LeftRegion or RightRegion comprising a nucleic acid sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively, a pol II promoter, e.g., EF-1, a human codon optimized chimeric Antigen Receptor (including an extracellular ligand binding domain, a transmembrane domain, and intracellular signaling domains), e.g., the CD19-specific Hu19-CD828Z (Genbank MN698642; Brudno et al. Nat Med 26:270-280 (2020)) CAR molecule, and a poly A tail. The template DNA is first mixed with purified integrase protein and incubated at room temperature for 15˜30 mins to form DNP complexes. Then, the DNP complex is nucleofected into activated T cells. Integration by the Gene Writer system is assayed using ddPCR for molecular quantification, and CAR expression is measured by flow cytometry.

### Example 18: Unidirectional Sequencing Assay for Determination of Integration Site

In this example, unidirectional sequencing is performed to determine the sequence of an unknown integration site with an unbiased profile of genome wide specificity.

Integration experiments are performed as in previous examples by using a Gene Writing system comprising an integrase and a template DNA for insertion. The integrase and donor plasmids are transfected into 293T cells. Genomic DNA is extracted at 72 hours post transfection and subjected to unidirectional sequencing according to the following method. First, a next generation library is created by fragmentation of the genomic DNA, end repair, and adaptor ligation. Next, fragmented genomic DNA harboring template DNA integration events is amplified by two-step nested PCR using forward primers binding to template specific sequence and reverse primers binding to sequencing adaptors. PCR products are visualized on a capillary gel electrophoresis instrument, purified, and quantified by Qubit (ThermoFisher). Final libraries are sequenced on a Miseq using 300 bp paired end reads (Illumina). Data analysis is performed by detecting the DNA flanking the insertion and mapping that sequence back to the human genome sequence, e.g., hg38.

### Example 19: Production of mRNA Encoding a Gene Writer Polypeptide

In this example, an integrase is expressed by in vitro transcription from mRNA. The mRNA template plasmid included the T7 promoter followed by the 5′UTR, the integrase coding sequence, the 3′ UTR, and ˜100 nucleotide long poly(A) tail. The plasmid is linearized by enzymatic restriction resulting in blunt end or 5′ overhang downstream of poly(A) tail and used for in vitro transcription (IVT) using T7 polymerase (NEB). Following IVT, the RNA is treated with DNase I (NEB). After buffer exchange, enzymatic capping is performed using Vaccinia capping enzyme (NEB) and 2′-O-methyltransferase (NEB) in the presence of GTP and SAM (NEB). The capped RNA is purified and concentrated using silica columns (for example, Monarch® RNA Cleanup kit) and buffered by 2 mM sodium citrate pH 6.5.

### Example 20: Use of Dual AAV Vector for the Treatment of Cystic Fibrosis in CFTR Mouse Model

In this example, a Gene Writing system is delivered as a dual AAV vector system for the treatment of cystic fibrosis in a mouse model of disease. Cystic fibrosis is a lung disease that is caused by mutations in the CTFR gene, which can be treated by the insertion of the wild-type CTFR gene into the genome of lung cells, such as cells found in the respiratory bronchioles and columnar non-ciliated cells in the terminal bronchiole.

A Gene Writing polypeptide, e.g., comprising an amino acid sequence of any of SEQ ID NOs: 1-12,677 (e.g., SEQ ID NOs: 1-11,432), and a template DNA comprising a cognate attachment site, e.g., an attB or attP site, e.g., a LeftRegion or RightRegion sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively, are packaged into AAV6 capsids with expression of the polypeptide driven by the CAG promoter, the combination of which has been shown to be effective for high level transduction and expression in murine respiratory epithelial cells according to the teachings of Halbert et al. Hum Gene Ther 18(4):344-354 (2007).

AAV preparations are co-delivered intranasally to CFTR gene knockout (Cftr^{tm1Unc}) mice (The Jackson Labs) using a modified intranasal administration, as described previously (Santry et al. BMC Biotechnol 17:43 (2017)). Briefly, AAVs are packaged, purified, and concentrated with either an integrase or template DNA, comprising the CFTR gene under the control of a pol II promoter, e.g., CAG promoter, and a cognate attachment site. In some embodiments, the CFTR expression cassette is flanked by the integrase attachment sites. Prepared AAVs are each delivered at a dose ranging from 1×10¹⁰-1×10¹² vg/mouse using a modified intranasal administration to the CFTR knockout mouse. After one week, lung tissue is harvested and used for genomic extraction and tissue analysis. To measure integration efficiency, CFTR gene integration is quantified using ddPCR to determine the fraction of cells and target sites containing or lacking the insertion. To assay expression from successfully integrated CFTR, tissue is analyzed by immunohistochemistry to determine expression and pathology.

### Example 21: Method of Treating Ornithine Transcarbamylase Deficiency Through the Introduction of Transiently Expressed Integrase

Ornithine transcarbamylase (OTC) deficiency is a rare genetic disorder that results in an accumulation of ammonia due to not having efficient breakdown of nitrogen. The accumulation of ammonia leads to hyperammonemia that can debilitating and in severe cases lethal. This example describes the treatment of OTC deficiency by the delivery and expression of an mRNA encoding a Gene Writer polypeptide, e.g., an integrase sequence of any of SEQ ID NOs: 1-12,677 (e.g., SEQ ID NOs: 1-11,432), along with the delivery of an AAV providing the template DNA for integration. The AAV template comprises a wild-type copy of the human OTC gene under the control of a pol II promoter, e.g., ApoE.hAAT, and a cognate attachment site, e.g., an attB or attP site, e.g., a LeftRegion or RightRegion sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively. In some embodiments, the OTC expression cassette is flanked by the integrase attachment sites.

In this example, LNP formulation of integrase mRNA follows the formulation of LNP-INT-01 (methods taught by Finn et al. Cell Reports 22:2227-2235 (2018), incorporated herein by reference) and template DNA is formulated in AAV2/8 (methods taught by Ginn et al. JHEP Reports (2019), incorporated herein by reference). Briefly, OTC deficiency is restored by treating neonatal Spf^{ush} mice (The Jackson Lab) by injecting LNP formulations (1-3 mg/kg) containing the integrase mRNA and AAV (1×10¹⁰-1×10¹² vg/mouse) containing the template DNA via the superficial temporal facial vein (Lampe et al. J Vis Exp 93:e52037 (2014)). The Spf^{ush} mouse has some residual mouse OTC activity which, in some embodiments, is silenced by the administration of an AAV that expresses an shRNA against mouse OTC as previously described (Cunningham et al. Mol Ther 19(5):854-859 (2011), the methods of which are incorporated herein by reference). OTC enzyme activity, ammonia levels, and orotic acid are measured as previously described (Cunningham et al. Mol Ther 19(5):854-859 (2011)). After 1 week, mouse livers are harvested and used for gDNA extraction and tissue analysis. The integration efficiency of hOTC is measured by ddPCR on extracted gDNA. Mouse liver tissue is analyzed by immunohistochemistry to confirm hOTC expression.

### Example 22: Use of a Gene Writing to Integrate a Large Payload into Human Cells

This example describes the integrase-mediated integration of a large payload into human cells in vitro.

In this example, the Gene Writer polypeptide component comprises an mRNA encoding an integrase, e.g., an integrase sequence comprising an amino acid sequence of any of SEQ ID NOs: 1-12,677 (e.g., SEQ ID NOs: 1-11,432), and a template DNA comprising: a cognate attachment site, e.g., an attB or attP site, e.g., a LeftRegion or RightRegion comprising a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively; a GFP expression cassette, e.g., a CMV promoter operably linked to EGFP; and stuffer sequence to bring the total plasmid size to approximately 20 kb.

Briefly, HEK293T cells are co-electroporated with the integrase mRNA and large template DNA. After three days, integration efficiency and specificity are measured. In order to measure efficiency of integration, droplet digital PCR (ddPCR) is performed on genomic DNA e.g., as described by Lin et al. Hum Gene Ther Methods 27(5):197-208 (2016), using primer-probe sets that amplify across the junction of integration, e.g., with one primer annealing to the template DNA and the other to an appropriate flanking region of the genome, such that only integration events are quantified. Data are normalized to an internal reference gene, e.g., RPP30, and efficiency is expressed as the average integration events per genome across the population of cells. To measure specificity, integration events in genomic DNA are assessed by unidirectional sequencing to determine genome coordinates, as described in Example 18.

### Example 23: Use of a Gene Writing to Integrate a Bacterial Artificial Chromosome into Human Embryonic Stem Cells Ex Vivo

This example describes the integrase-mediated integration of a bacterial artificial chromosome (BAC) into human embryonic stem cells (hESCs).

BAC vectors are capable of maintaining extremely large (>100 kb) DNA payloads, and thus can carry many genes or complex gene circuits that may be useful in cellular engineering. Though there has been demonstration of their integration into hESCs (Rostovskaya et al. Nucleic Acids Res 40(19):e150 (2012)), this was accomplished using transposons that lack sequence specificity in their integration patterns. This Example describes sequence-specific integration of large constructs.

In this example, a BAC engineered to carry the desired payload further comprises an attachment site, e.g., an attB or attP site, e.g., a LeftRegion or RightRegion comprising a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively, that enables recognition by the Gene Writer polypeptide, e.g., an integrase, e.g., an integrase comprising an amino acid sequence of any of SEQ ID NOs: 1-12,677 (e.g., SEQ ID NOs: 1-11,432). An approximately 150 kb BAC is introduced into hESCs by electroporation or lipofection as per the teachings of Rostovskaya et al. Nucleic Acids Res 40(19):e150 (2012). After three days, integration efficiency and specificity are measured. In order to measure efficiency of integration, droplet digital PCR (ddPCR) is performed on genomic DNA e.g., as described by Lin et al. Hum Gene Ther Methods 27(5):197-208 (2016), using primer-probe sets that amplify across the junction of integration, e.g., with one primer annealing to the template DNA and the other to an appropriate flanking region of the genome, such that only integration events are quantified. Data are normalized to an internal reference gene, e.g., RPP30, and efficiency is expressed as the average integration events per genome across the population of cells. To measure specificity, integration events in genomic DNA are assessed by unidirectional sequencing to determine genome coordinates, as described in Example 18.

### Example 24: Use of Dual AAV Vector to Integrate a Transgene into a Mouse Model that Contains an Integrase Landing Pad Site

Integrase proteins are found naturally in bacteriophage and utilize a sequence of the phage genome (attP) to integrate the part of its genome into a bacteria's genome at a specific sequence (attB). Integrase proteins can be utilized as drivers to integrate DNA into a genome when supplied with a donor vector carrying an insert DNA that bears an appropriate recognition sequence (e.g. attP or attB) and the target or host genome bears a corresponding recognition sequence (e.g. attB or attP). This requirement for a specific sequence to be found in the host genome to have efficient integration can limit the use and/or efficacy of an integrase to insert a transgene into the genome of a mouse, making it challenging to create a mouse model or treat a disease found in the background of a mouse genetic disease model. In this example, a mouse engineered to have an attP recognition site (e.g., attP sequence for Bxb1 integrase) in its genome is used to demonstrate targeted integration by delivery of 1) an insert DNA that bears a sequence of interest and further comprises an attB recognition site (e.g., attB sequence for Bxb1 integrase) and 2) an integrase (e.g., Bxb1 integrase) that catalyzes the integration of the insert DNA into the genomic attP site. Further, in this example, the Bxb1-specific attP and attB recognition sequences used have the central dinucleotide changed from GT to GA. In some examples, the DNA sequence of interest is a heterologous object sequence comprising an RNA polymerase II promoter sequence (e.g., Human thyroxine binding globulin, TBG) and the DNA coding region of a therapeutic protein or a reporter gene (e.g., Renilla renformis luciferase).

Briefly, AAVs (e.g., AAV-DJ) are packaged, purified, and concentrated with either a construct comprising DNA encoding an integrase protein (e.g., Bxb1) or comprising the insert DNA (e.g., Renilla renformis luciferase under the control of TBG promoter and the described attB sequence). Mice with a stable integration of the attP recognition sequence are co-administered one or both of the two AAV viruses via intraperitoneal injection at doses ranging from 1×10¹⁰-1×10¹³ vg per virus per mouse. The integration is monitored over time by unidirectional sequencing of livers, among other organs, as previously described. In-life imaging of the luciferase expression is monitored as previously described (Bhaumik, S., & Gambhir, S. S., PNAS 2002, https://doi.org/10.1073/pnas.012611099).

### Example 25: Treatment of Multiple Diseases with a Single Composition Incorporating Multiple Genes

Ornithine transcarbamylase (OTC) deficiency and Citrullinemia type I are distinct diseases caused by mutations in different genes (OTC and ASS1, respectively) that both result in disruption of the urea cycle, ultimately leading to the accumulation of nitrogen (as ammonia) in the blood. The accumulation of ammonia leads to hyperammonemia, which can ultimately cause tissue and neurotoxicity with debilitating and potentially fatal consequences.

This example describes the design and use of a single Gene Writing system that can be provided for treatment of more than one disease. More specifically, this example describes the treatment of OTC deficiency or Citrullinemia type I by the delivery and expression of an mRNA encoding a Gene Writer polypeptide, e.g., an integrase sequence of any of SEQ ID NOs: 1-12,677 (e.g., SEQ ID NOs: 1-11,432), and an AAV comprising a template DNA for integration.

The template DNA in this example comprises functional copies of both the human OTC and ASS1 genes separated by a self-cleaving peptide (for example 2A) under the control of a pol II promoter, e.g., ApoE.hAAT, and a cognate attachment site, e.g., an attB or attP site, e.g., a LeftRegion or RightRegion sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432). In some embodiments, the expression cassette comprising both OTC and ASS1 is flanked by integrase attachment sites. The composition described is used to treat either OTC deficiency or Citrullinemia type I.

In this example, LNP formulation of integrase mRNA follows the formulation of LNP-INT-01 (methods taught by Finn et al. Cell Reports 22:2227-2235 (2018), incorporated herein by reference) and template DNA is packaged in AAV2/8 (methods taught by Ginn et al. JHEP Reports (2019), incorporated herein by reference). Briefly, OTC deficiency is restored by treating neonatal Spf^{ush} mice (The Jackson Lab) by injecting LNP formulations (1-3 mg/kg) containing the integrase mRNA and AAV (1×10¹⁰-1×10¹² vg/mouse) containing the template DNA via the superficial temporal facial vein (Lampe et al. J Vis Exp 93:e52037 (2014)). The Spf^{ush} mouse has some residual mouse OTC activity which, in some embodiments, is silenced by the administration of an AAV that expresses an shRNA against mouse OTC as previously described (Cunningham et al. Mol Ther 19(5):854-859 (2011), the methods of which are incorporated herein by reference). OTC enzyme activity, ammonia levels, and orotic acid are measured as previously described (Cunningham et al. Mol Ther 19(5):854-859 (2011)). After 1 week, mouse livers are harvested and used for gDNA extraction and tissue analysis. The integration efficiency of hOTC is measured by ddPCR on extracted gDNA. Mouse liver tissue is analyzed by immunohistochemistry to confirm hOTC expression.

In some embodiments, the same composition described and used to treat a model of OTC deficiency above may also be used to treat Citrullinemia type I. Briefly, ASS1 deficiency is restored by treating a neonatal lethal argininosuccinate synthetase (ASS) knockout mouse model (Cindy Y Kok et al, Mol Ther. 21(10):1823-1831 (2013), the methods of which are incorporated herein by reference in their entirety) using the described LNP and AAV. Specifically, ASS knockout mice are injected with LNP formulations (1-3 mg/kg) containing the integrase mRNA and AAV (1×10¹⁰-1×10¹² vg/mouse) containing the template DNA via the superficial temporal facial vein (Lampe et al. J Vis Exp 93:e52037 (2014)). Ammonia levels, orotic acid and overall mice survival are measured as previously described (Cindy Y Kok et al, Mol Ther.

21(10):1823-1831 (2013)). After 2-4-8 weeks, mouse livers are harvested and used for gDNA extraction and tissue analysis. The integration efficiency of hASS1 is measured by ddPCR on extracted gDNA. Mouse liver tissue is analyzed by immunohistochemistry to confirm hASS1 expression.

In some embodiments, the Gene Writing system integrates the OTC-ASS1 expression cassette into OTC deficiency and ASS1 knockout mouse models. This same system thus restores healthy urea cycles in both models. In some embodiments, blood ammonia levels are reduced from hyperammonemia to normal levels, e.g., OTC deficiency or ASS1 knockout mice treated with the Gene Writing system show at least a 2, 5, 10, 50, or at least a 100-fold reduction in blood ammonia levels relative to control mice. In some embodiments, orotic acid levels are reduced from elevated to normal levels, e.g., OTC deficiency or ASS1 knockout mice treated with the Gene Writing system show at least a 2, 5, 10, 50, or at least a 100-fold reduction in orotic acid levels relative to control mice.

### Example 26: Selection of Lipid Reagents with Reduced Aldehyde Content

In this example, lipids are selected for downstream use in lipid nanoparticle formulations containing Gene Writing component nucleic acid(s), and lipids are selected based at least in part on having an absence or low level of contaminating aldehydes. Reactive aldehyde groups in lipid reagents may cause chemical modifications to component nucleic acid(s), e.g., RNA, e.g., template RNA, during LNP formulation. Thus, in some embodiments, the aldehyde content of lipid reagents is minimized.

Liquid chromatography (LC) coupled with tandem mass spectrometry (MS/MS) can be used to separate, characterize, and quantify the aldehyde content of reagents, e.g., as described in Zurek et al. The Analyst 124(9):1291-1295 (1999), incorporated herein by reference. Here, each lipid reagent is subjected to LC-MS/MS analysis. The LC/MS-MS method first separates the lipid and one or more impurities with a C8 HPLC column and follows with the detection and structural determination of these molecules with the mass spectrometer. If an aldehyde is present in a lipid reagent, it is quantified using a staple-isotope labeled (SIL) standard that is structurally identical to the aldehyde, but is heavier due to C13 and N15 labeling. An appropriate amount of the SIL standard is spiked into the lipid reagent. The mixture is then subjected to LC-MS/MS analysis. The amount of contaminating aldehyde is determined by multiplying the amount of SIL standard and the peak ratio (unknown/SIL). Any identified aldehyde(s) in the lipid reagents is quantified as described. In some embodiments, lipid raw materials selected for LNP formulation are not found to contain any contaminating aldehyde content above a chosen level. In some embodiments, one or more, and optionally all, lipid reagents used for formulation comprise less than 3% total aldehyde content. In some embodiments, one or more, and optionally all, lipid reagents used for formulation comprise less than 0.3% of any single aldehyde species. In some embodiments, one or more, and optionally all, lipid reagents used in formulation comprise less than 0.3% of any single aldehyde species and less than 3% total aldehyde content.

### Example 27: Quantification of RNA Modification Caused by Aldehydes During Formulation

In this example, the RNA molecules are analyzed post-formulation to determine the extent of any modifications that may have happened during the formulation process, e.g., to detect chemical modifications caused by aldehyde contamination of the lipid reagents (see, e.g., Example 26).

RNA modifications can be detected by analysis of ribonucleosides, e.g., as according to the methods of Su et al. Nature Protocols 9:828-841 (2014), incorporated herein by reference in its entirety. In this process, RNA is digested to a mix of nucleosides, and then subjected to LC-MS/MS analysis. RNA post-formulation is contained in LNPs and must first be separated from lipids by coprecipitating with GlycoBlue in 80% isopropanol. After centrifugation, the pellets containing RNA are carefully transferred to a new Eppendorf tube, to which a cocktail of enzymes (benzonase, Phosphodiesterase type 1, phosphatase) is added to digest the RNA into nucleosides. The Eppendorf tube is placed on a preheated Thermomixer at 37° C. for 1 hour. The resulting nucleosides mix is directly analyzed by a LC-MS/MS method that first separates nucleosides and modified nucleosides with a C18 column and then detects them with mass spectrometry.

If aldehyde(s) in lipid reagents have caused chemical modification, data analysis will associate the modified nucleoside(s) with the aldehyde(s). A modified nucleoside can be quantified using a SIL standard which is structurally identical to the native nucleoside except heavier due to C13 and N15 labeling. An appropriate amount of the SIL standard is spiked into the nucleoside digest, which is then subjected to LC-MS/MS analysis. The amount of the modified nucleoside is obtained by multiplying the amount of SIL standard and the peak ratio (unknown/SIL). LC-MS/MS is capable of quantifying all the targeted molecules simultaneously.

In some embodiments, the use of lipid reagents with higher contaminating aldehyde content results in higher levels of RNA modification as compared to the use of higher purity lipid reagents as materials during the lipid nanoparticle formulation process. Thus, in preferred embodiments, higher purity lipid reagents are used that result in RNA modification below an acceptable level.

### Example 28: Gene Writers for Integration of a CAR in T-Cells In Vivo

This example describes a Gene Writer™ genome editing system delivered T-cells in vivo for integration and stable expression of a genetic payload. Specifically, targeted nanoparticles are used to deliver a Gene Writing system capable of integrating a chimeric antigen receptor (CAR) expression cassette into the genome of T-cells to generate CAR-T cells in a murine model.

In this example, a Gene Writing system comprises an mRNA encoding a Gene Writing polypeptide, e.g., a recombinase enzyme described herein, and an insert DNA comprising a recombinase recognition site and a transgene cassette, wherein the transgene cassette comprises the coding sequence for the CD19-specific m194-1BBz CAR driven by the EF1a promoter (Smith et al. Nat Nanotechnol 12(8):813-820 (2017)). In order to achieve delivery specifically to T-cells, targeted LNPs (tLNPs) are generated that carry a conjugated mAb against CD4. See, e.g., Ramishetti et al. ACS Nano 9(7):6706-6716 (2015). Alternatively, conjugating a mAb against CD3 can be used to target both CD4+ and CD8+ T-cells (Smith et al. Nat Nanotechnol 12(8):813-820 (2017)). In other embodiments, the nanoparticle used to deliver to T-cells in vivo is a constrained nanoparticle that lacks a targeting ligand, as taught by Lokugamage et al. Adv Mater 31(41):e1902251 (2019).

The tLNP can be made by first preparing the nucleic acid mix (e.g., polypeptide mRNA: template DNA molar ratio of 1:40) with a mixture of lipids (cholesterol, DSPC, PEG-DMG, Dlin-MC3-DMA, and DSPE-PEG-maleimide) and then chemically conjugating the desired DTT-reduced mAb (e.g., anti-CD4, e.g., clone YTS.177) to the maleimide functional group on the LNPs. See Ramishetti et al. ACS Nano 9(7):6706-6716 (2015).

Six to 8 week old C57BL6/J mice are injected intravenously with formulated LNP at a dose of 1 mg RNA/kg body weight. Blood is collected at one day and three days post-administration in heparin-coated collection tubes, and the leukocytes are isolated by density centrifugation using Ficoll-Paque PLUS (GE Healthcare). Five days post-administration, animals are euthanized and blood and organs (spleen, lymph nodes, bone marrow cells) are harvested for T-cell analysis. Expression of the anti-CD19 CAR is detected by FACS using specific immunological sorting. Positive cells are confirmed for integration by methods as described herein, e.g., molecular combing or Q-FISH.

### Example 29: Characterization of Integration Sites by Molecular Combing

AAV genomes are known to undergo multiple mechanisms of intra and intermolecular recombination after delivery to cells (McCarty et al Annu Rev Genet 38:819-45 (2004)). Since an insert DNA may be delivered via an AAV vector, it is possible that in this context, some of the molecules may occur as concatemers, and when used as a substrate for Gene Writing, these concatemeric insert DNA molecules may result in the integration of more than one copy of the original insert DNA. It may thus be useful to analyze the fraction of integration events that result in single vs concatemeric insertions of the template DNA, the average number of copies per integration site, and the orientation of concatemeric molecules, e.g., the frequency of head-to-head or head-to-tail conformations. This example describes the use of molecular combing technology to determine the configuration of integration sites after AAV-mediated delivery of a Gene Writing system in human cells.

The Bxb1 recombinase (e.g., SEQ ID NO: 11,636) is an enzyme that has been used to integrate DNA in human cells that have been modified to contain an appropriate recognition site in the genome, and is used here as a representative example of recombinase systems disclosed herein. In this example, HEK293T landing pad cell lines are generated by single copy infection with a lentiviral vector containing the BXB1 attP-attP* site. To perform the recombinase-mediated integration, single copy landing pad cells are first seeded in a 48-well plate at ˜40,000 cells/well. At ˜24 hr post-seeding, adeno-associated viral vectors containing the BXB1 attB* donor (cognate recognition site to the attP* site in the landing pad) are transduced with an AAV containing an insert DNA in the presence or absence of a second AAV comprising the coding sequence for Bxb1 integrase. 2 weeks post transduction, ˜10% of the AAV transduced cells are harvested and gDNA is analyzed using a ddPCR assay specific to the landing pad site to confirm integration (% CNV/landing pad). Methods for molecular combing follow the approach of Kaykov et al Sci Rep 6:19636 (2016), incorporated herein by reference in its entirety. Briefly, ˜300,000 transduced cells from each transduced sample are extracted for high molecular weight genomic DNA into an agarose plug. Genomic DNA molecules are then mechanically stretched and aligned in a controlled and consistent manner on the glass surface, enabling precise and direct measurements along the length of the DNA fiber. In-situ hybridization is performed using prelabeled DNA probes that enable visualization for integration site configuration analysis. Probes for the Bxb1 attP-attP* landing pad (target site), AAV Bxb1 attB*donor sequence (insert DNA), and reference gene RPP30 are labeled using three distinct colors for differentiating the signal from each probe. Post hybridization, fluorescence signals are acquired and quantified. By this method, the number and location of the distinct fluorescence signals relative to each other provide a view of the insert copy number and orientation within integrated DNA.

### Example 30: Determination of Integration Sites by Inverse PCR

This example describes the characterization of integration sites for a Gene Writer system. In some embodiments, a Gene Writer system may exhibit exquisite specificity for a single target site or target sequence. In other embodiments, a Gene Writer system may have a more relaxed specificity and catalyze integration of an insert DNA at a variety of locations in the genome. Thus, for any given Gene Writer, it is useful to determine the breadth of its integration profile.

In this example, a Gene Writing system is used to modify the genome of HEK293T cells as described in any of the preceding Examples. After transfection, HEK293T cells are cultured for at least 4 days and then assayed for site-specific genome editing. Genomic DNA is first digested with pairs of restriction enzymes that generate incompatible cohesive ends and that cut at least once in the insert DNA, and then self-ligated to generate circular DNA ideally comprising both insert DNA and flanking genomic DNA. Inverted PCR amplification is conducted as described in Olivares et al Nat Biotechnol 20:1124-1128 (2002), the methods of which are incorporated herein by reference in their entirety, using forward and reverse primers specific to the insert DNA that will result in amplification of adjacent genomic DNA. Amplified PCR products are then sequenced using next generation sequencing technology on a MiSeq instrument. For sequence analysis, MiSeq reads are mapped to the HEK293T genome to identify locations of integration. In some embodiments, a Gene Writer system described herein results in detectable integration at a single site. In some embodiments, a Gene Writer system described herein results in detectable integration at a limited number of sites, e.g., less than 100, 90, 80, 70, 60, 50, 45, 40, 35, 30, 25, 20, 15, 10, 9, 8, 7, 6, 5, 4, 3, or less than 2 sites. In other embodiments, a Gene Writer system described herein results in detectable integration at more than 100 sites.

### Example 31: Production of IDLV-Recombinase Viral Particles Using a Lentiviral Packaging System

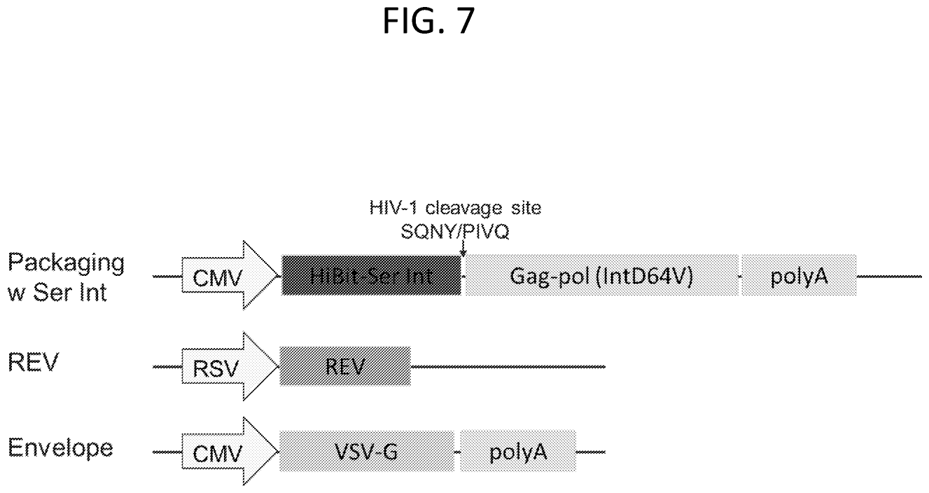

This example describes methods used for producing IDLV vectors as described herein (e.g., as shown in FIGS. 6-7). In brief, a template plasmid comprising (1) IDLV elements, optionally including a recombinase coding sequence, as described herein, (2) a DNA recognition site, and (3) a heterologous object sequence, e.g., an expression element further comprising a promoter and transgene for producing a therapeutic protein, is packaged into lentiviral-based particles. Briefly, the IDLV-recombinase plasmid and the three packaging plasmids are co-transfected into HEK293T cells using the TransIT®-293 transfection reagent (Mirus Bio), as according to manufacturer's instructions. Medium is replaced at 4 hrs post-transfection. At 48 hours post-transfection, culture supernatants are collected and concentrated by ultracentrifugation. IDLV-recombinase viral titers are determined by droplet digital PCR (ddPCR), e.g., as performed in Example 5 or in Lin et al., Human Gene Therapy Methods 27(5):197-208 (2016), using primers and probes specific to the IDLV-recombinase sequence. To verify expression of the heterologous recombinase, HEK293T cells are transduced with the IDLV-recombinase particles as generated above and are harvested at 4, 24, and 48 hr post-transduction to monitor expression of the recombinase via Western blot with an antibody against the recombinase or against a peptide tag fused to the recombinase, e.g., a HiBit tag. In some embodiments, expression of recombinase comprising a HiBit tag fusion is instead detected by Western blot using a HiBit Blotting System (Promega) or in lysate using a HiBit Lytic Detection System (Promega), as according to manufacturer's instructions.

### Example 32. Modified IDLV Systems Further Comprising Heterologous Recombinase Functionality Yield Improved Stability of Expression

This example demonstrates the improved stability of transgene expression achieved in target cells using an IDLV-recombinase system as compared to a non-integrative IDLV system. This enhancement in stability is conceived to occur as the result of stable integration by the recombinase system, which leads to improved maintenance as part of normal genomic DNA replication, whereas non-integrative systems are not replicated and lost through cell division and dilution. In order to determine the stability properties of IDLV-recombinase systems, cells are treated with reporter cassettes comprised in IDLV-recombinase systems or non-integrative systems and reporter expression is measured over time to determine stability.

To assess transgene stability as introduced by IDLV-recombinase systems, HEK293T and HepG2 cells are transfected with the following vectors: (1) standard IDLV plasmid for expressing an LTR-flanked template encoding an EGFP reporter; (2) a recombinase expression plasmid and the IDLV plasmid that further comprises a DNA recognition sequence for the recombinase, as described herein; or (3) a recombinase expression plasmid and the IDLV plasmid lacking the DNA recognition sequence of (2). At 3 days post-transfection, transfection efficiency is determined as the frequency of GFP+ cells, as measured by flow cytometry. Fluorescence is subsequently monitored by flow cytometry on days 7, 10, 14, 21, 28, and 60 post-transfection. The integration frequency is approximated by determining stable expression of EGFP at day 21, e.g., the fraction of cells that are GFP+ by flow cytometry at day 21. Stability profiles of transgene expression for each system are determined by assaying the frequency (e.g., percent GFP+) and/or the expression level (e.g., median fluorescence) of cells at days 28 and 60 post-transduction, as measured by flow cytometry for EGFP fluorescence.

In some embodiments, cells treated with a complete IDLV-recombinase system (2) show a decrease in the loss of frequency of expression (e.g., percent GFP+) and/or loss of reporter expression (e.g., median fluorescence) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than cells treated with (1) and/or (3). In some embodiments, cells treated with a complete IDLV-recombinase system (2) show a higher frequency of expression (e.g., percent GFP+) and/or a higher level of expression (e.g., median fluorescence) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold higher than cells treated with (1) and/or (3).

As described in this example, a recombinase may be encoded by a sequence of any of SEQ ID NOs: 1-12,677 (e.g., SEQ ID NOs: 1-11,432 and the DNA recognition sequence may be comprised in a LeftRegion or RightRegion of any of SEQ ID NOs: 13,001-25,677 (e.g., SEQ ID NOs: 13,001-24,432) or SEQ ID NOs: 26,001-38,677 (e.g., SEQ ID NOs: 26,001-37,432), respectively of a corresponding line number of the sequence encoding the recombinase. In some embodiments, the cells used comprise an endogenous target sequence that is also recognized by the recombinase, such that the recombinase is capable of catalyzing the recombination of the DNA recognition sequence with the endogenous target sequence. In some embodiments, a recombinase without an endogenous target site in the native genome is used for the purpose of experimentation by first integrating an appropriate target site into the genome, e.g., by creating a landing pad comprising the target site. As an example, a landing pad cell line may be generated by transducing HEK293T cells with a lentiviral vector comprising the attB site of the recombinase, e.g., a recombinase of SEQ ID NO: 11,636, comprising the attB sequence 5′-GGCTTGTCGACGACGGCGGTCTCCGTCGTCAGGATCAT-3′. Accordingly, the recombinase is encoded by a sequence comprising a sequence of SEQ ID NO: 11,636 and the DNA recognition sequence of the template plasmid comprises a sequence from the LeftRegion or RightRegion, (e.g., SEQ ID NOs: 24,636 and 37,636, respectively), e.g., comprises the sequence 5′-GGTTTGTCTGGTCAACCACCGCGGTCTCAGTGGTGTACGGTACAAACC-3′.

### Example 33: Determination of IDLV-Recombinase Integration Frequency Using Droplet Digital PCR

This example describes the molecular measurement of integration frequency of IDLV-recombinase systems. Depending on the recombinase and DNA recognition sequence used, as well as the cell type being transfected, an IDLV-recombinase system may integrate with differing levels of efficiency. Thus, it is desirable to determine the average number of integrations per cell in a population of cells as a metric for efficiency.

In this example, IDLV-recombinase viral particles are first transduced into cells, e.g., HEK293 or HepG2 cells, as in Example 32. Transfected cells are harvested for genomic DNA extraction at various timepoints, e.g., at days 1, 2, 3, 4, and 7 post-transduction. Droplet digital PCR (ddPCR) assays are developed to determine the integration frequency at the 5′ junction and 3′ junction of integration events. For the 5′ junction, the primer and probe sets include a forward primer binding to ˜150 bp upstream of the junction in the flanking genomic DNA, a FAM-labeled probe binding to ˜50 bp downstream of the junction in the IDLV-recombinase sequence, and a reverse primer binding to ˜150 bp downstream of the junction in the IDLV-recombinase sequence. For the 3′ junction, the primer and probe sets include a forward primer binding to ˜150 bp upstream of the junction in the IDLV-recombinase sequence, a FAM-labeled probe binding to ˜50 bp upstream of the junction in the IDLV-recombinase sequence, and a reverse primer binding to ˜150 bp downstream of the junction in the flanking genomic DNA. Optimized assays including the described 5′ junction assay and 3′ junction assay are performed to determine the frequency of IDLV-recombinase integration.

In some embodiments, an IDLV-recombinase system results in a frequency of integration of at least approximately 0.1, 0.2, 0.25, 0.5, 1.0, 1.5, 2.0, 2.5, 3, 4, 5, 6, 7, 8, 9, or at least 10 copies per genome. In some embodiments, an IDLV-recombinase system results in a frequency of integration of no more than 10, 9, 8, 7, 6, 5, 4, 3, 2, or no more than 1 copy per genome. In some embodiments, an IDLV-recombinase system results in a similar efficiency of integration as measure by comparing 5′ and 3′ junction assays, e.g., results in no more than 1.1, 1.2, 1.3, 1.4, 1.5, 1.6, 1.7, 1.8, 1.9, or 2.0-fold change in frequency of integration when comparing the results from 5′ and 3′ junction assays.

### Example 34. Assessing Sequence Specificity of IDLV-Recombinase Via Uniseq

This example describes determination of the integration specificity of an IDLV-recombinase system. Specifically, a unidirectional sequencing assay is employed in order to determine the sequences and locations of all IDLV-recombinase-mediated integration events occurring in the genome. This assay provides an unbiased profile of genome-wide specificity.

To conduct integration sequence profiling experiments, IDLV-recombinase systems, as described herein, or unmodified IDLV systems incapable of integration, are transfected into HEK293T cells as in Example 32. Genomic DNA is harvested at 14 days post-transduction and is subjected to unidirectional sequencing according to the protocol described. First, a next generation library is created by fragmentation of the genomic DNA followed by end repair and adaptor ligation. Next, genomic DNA fragments harboring IDLV-specific integrations are amplified by a two-step nested PCR approach using one of two sets of primers: 1) forward primers binding to the universal primer region U1 and reverse primers binding to the universal primer region U2 to detect integration mediated by the recombinase (see FIG. 8) or 2) forward primers binding to the 3′ LTR and reverse primers binding to the 5′ LTR to detect any IDLV integration events. PCR products are purified using AMPure XP beads (Beckman), visualized on a capillary gel electrophoresis instrument, and quantified by qPCR. Final libraries are sequenced on an Illumina MiSeq using 300 bp paired-end reads (Illumina), as according to manufacturer's instructions. Data are analyzed by mapping junction reads to a reference genome (e.g., hg38) and determining sites of integration.

In some embodiments, an IDLV-recombinase system is found to integrate into no more than 10000 unique genomic sites, e.g., nor more than 10000, 9000, 8000, 7000, 6000, 5000, 4000, 3000, 2000, 1000, 900, 800, 700, 600, 500, 400, 300, 200, or no more than 100 unique genomic sites. In some embodiments, an IDLV-recombinase system is found to integrate into no more than 100 unique genomic sites, e.g., nor more than 100, 90, 80, 70, 60, 50, 40, 30, 20, or no more than 10 unique genomic sites. In preferred embodiments, an IDLV-recombinase system is found to integrate into no more than 10 unique genomic sites, e.g., no more than 10, 9, 8, 7, 6, 5, 4, 3, 2, or no more than 1 unique genomic site. In some embodiments, an IDLV-recombinase system is found to integrate into at least 1 unique genomic site, e.g., at least 1, 2, 3, 4, 5, 6, 7, 8, 9, 10, 20, 30, 40, 50, 60, 70, 80, 90, 100, 200, 300, 400, 500, 600, 700, 800, 900, 1000, 2000, 3000, 4000, 5000, 6000, 7000, 8000, 9000, or at least 10000 unique genomic sites.

### Example 35: Selection of Lipid Reagents with Reduced Aldehyde Content

In this example, lipids are selected for downstream use in lipid nanoparticle formulations containing Gene Writing component nucleic acid(s), and lipids are selected based at least in part on having an absence or low level of contaminating aldehydes. Reactive aldehyde groups in lipid reagents may cause chemical modifications to component nucleic acid(s), e.g., RNA, e.g., template RNA, during LNP formulation. Thus, in some embodiments, the aldehyde content of lipid reagents is minimized.

Liquid chromatography (LC) coupled with tandem mass spectrometry (MS/MS) can be used to separate, characterize, and quantify the aldehyde content of reagents, e.g., as described in Zurek et al. The Analyst 124(9):1291-1295 (1999), incorporated herein by reference. Here, each lipid reagent is subjected to LC-MS/MS analysis. The LC/MS-MS method first separates the lipid and one or more impurities with a C8 HPLC column and follows with the detection and structural determination of these molecules with the mass spectrometer. If an aldehyde is present in a lipid reagent, it is quantified using a staple-isotope labeled (SIL) standard that is structurally identical to the aldehyde but is heavier due to C13 and N15 labeling. An appropriate amount of the SIL standard is spiked into the lipid reagent. The mixture is then subjected to LC-MS/MS analysis. The amount of contaminating aldehyde is determined by multiplying the amount of SIL standard and the peak ratio (unknown/SIL). Any identified aldehyde(s) in the lipid reagents is quantified as described. In some embodiments, lipid raw materials selected for LNP formulation are not found to contain any contaminating aldehyde content above a chosen level. In some embodiments, one or more, and optionally all, lipid reagents used for formulation comprise less than 3% total aldehyde content. In some embodiments, one or more, and optionally all, lipid reagents used for formulation comprise less than 0.3% of any single aldehyde species. In some embodiments, one or more, and optionally all, lipid reagents used in formulation comprise less than 0.3% of any single aldehyde species and less than 3% total aldehyde content.

### Example 36: Quantification of RNA Modification Caused by Aldehydes During Formulation

In this example, the RNA molecules are analyzed post-formulation to determine the extent of any modifications that may have happened during the formulation process, e.g., to detect chemical modifications caused by aldehyde contamination of the lipid reagents (see, e.g., Example 38).

RNA modifications can be detected by analysis of ribonucleosides, e.g., as according to the methods of Su et al. Nature Protocols 9:828-841 (2014), incorporated herein by reference in its entirety. In this process, RNA is digested to a mix of nucleosides, and then subjected to LC-MS/MS analysis. RNA post-formulation is contained in LNPs and must first be separated from lipids by coprecipitating with GlycoBlue in 80% isopropanol. After centrifugation, the pellets containing RNA are carefully transferred to a new Eppendorf tube, to which a cocktail of enzymes (benzonase, Phosphodiesterase type 1, phosphatase) is added to digest the RNA into nucleosides. The Eppendorf tube is placed on a preheated Thermomixer at 37° C. for 1 hour. The resulting nucleosides mix is directly analyzed by a LC-MS/MS method that first separates nucleosides and modified nucleosides with a C18 column and then detects them with mass spectrometry.

If aldehyde(s) in lipid reagents have caused chemical modification, data analysis will associate the modified nucleoside(s) with the aldehyde(s). A modified nucleoside can be quantified using a SIL standard which is structurally identical to the native nucleoside except heavier due to C13 and N15 labeling. An appropriate amount of the SIL standard is spiked into the nucleoside digest, which is then subjected to LC-MS/MS analysis. The amount of the modified nucleoside is obtained by multiplying the amount of SIL standard and the peak ratio (unknown/SIL). LC-MS/MS is capable of quantifying all the targeted molecules simultaneously.

In some embodiments, the use of lipid reagents with higher contaminating aldehyde content results in higher levels of RNA modification as compared to the use of higher purity lipid reagents as materials during the lipid nanoparticle formulation process. Thus, in preferred embodiments, higher purity lipid reagents are used that result in RNA modification below an acceptable level.

### Example 37: Formulation of Lipid Nanoparticles Encapsulating Firefly Luciferase mRNA

In this example, a reporter mRNA encoding firefly luciferase was formulated into lipid nanoparticles comprising different ionizable lipids. Lipid nanoparticle (LNP) components (ionizable lipid, helper lipid, sterol, PEG) were dissolved in 100% ethanol with the lipid component. These were then prepared at molar ratios of 50:10:38.5:1.5 using ionizable lipid LIPIDV004 or LIPIDVO05 (Table 23), DSPC, cholesterol, and DMG-PEG 2000, respectively. Firefly Luciferase mRNA-LNPs containing the ionizable lipid LIPIDVO03 (Table 23) were prepared at a molar ratio of 45:9:44:2 using LIPIDV003, DSPC, cholesterol, and DMG-PEG 2000, respectively. Firefly luciferase mRNA used in these formulations was produced by in vitro transcription and encoded the Firefly Luciferase protein, further comprising a 5′ cap, 5′ and 3′ UTRs, and a polyA tail. The mRNA was synthesized under standard conditions for T7 RNA polymerase in vitro transcription with co-transcriptional capping, but with the nucleotide triphosphate UTP 100% substituted with Ni-methyl-pseudouridine triphosphate in the reaction. Purified mRNA was dissolved in 25 mM sodium citrate, pH 4 to a concentration of 0.1 mg/mL.

Firefly Luciferase mRNA was formulated into LNPs with a lipid amine to RNA phosphate (N:P) molar ratio of 6. The LNPs were formed by microfluidic mixing of the lipid and RNA solutions using a Precision Nanosystems NanoAssemblr™ Benchtop Instrument, using the manufacturer's recommended settings. A 3:1 ratio of aqueous to organic solvent was maintained during mixing using differential flow rates. After mixing, the LNPs were collected and dialyzed in 15 mM Tris, 5% sucrose buffer at 4° C. overnight. The Firefly Luciferase mRNA-LNP formulation was concentrated by centrifugation with Amicon 10 kDa centrifugal filters (Millipore). The resulting mixture was then filtered using a 0.2 m sterile filter. The final LNP was stored at −80° C. until further use.

TABLE 23
Exemplary Ionizable Lipids

| | | Molecular | |
|---|---|---|---|
| LIPID ID | Chemical Name | Weight | Structure |
| LIPIDV003 | (9Z,12Z)-3-((4,4- bis(octyloxy)butanoyl) oxy)-2-((((3-(diethylamino) propoxy)carbonyl) oxy)methyl)propyl octadeca-9,12-dienoate | 852.29 | 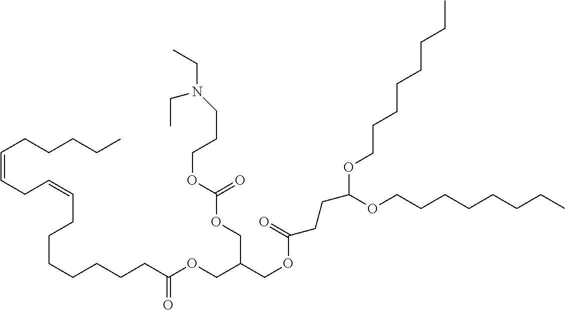 [SMILES: CCCCC/C=C\C/C=C\CCCCCCCC(=O)OCC(COC(=O)CCC(OCCCCCCCC)OCCCCCCCC)COC(=O)OCCCN(CC)CC] |
| LIPIDV004 | Heptadecan-9-yl 8- ((2-hydroxy-ethyl)(8- (nonyloxy)-8- oxooctyl)amino) octanoate | 710.18 | 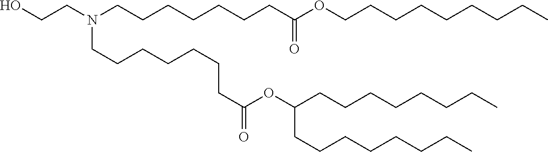 [SMILES: CCCCCCCCCOC(=O)CCCCCCCN(CCO)CCCCCCCC(=O)OC(CCCCCCCC)CCCCCCCC] |
| LIPIDV005 | | 919.56 | 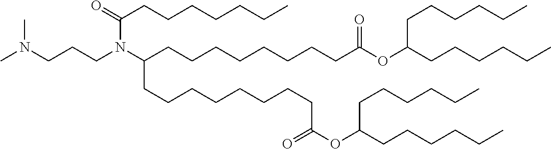 [SMILES: CCCCCCCC(=O)N(CCCN(C)C)C(CCCCCCCCC(=O)OC(CCCCCC)CCCCCC)CCCCCCCCC(=O)OC(CCCCCC)CCCCCC] |

Prepared LNPs were analyzed for size, uniformity, and % RNA encapsulation. The size and uniformity measurements were performed by dynamic light scattering using a Malvern Zetasizer DLS instrument (Malvern Panalytical). LNPs were diluted in PBS prior to being measured by DLS to determine the average particle size (nanometers, nm) and polydispersity index (pdi). The particle sizes of the Firefly Luciferase mRNA-LNPs are shown in Table 24.

TABLE 24
LNP particle size and uniformity

| LNP ID | Ionizable Lipid | Particle Size (nm) | pdi |
|---|---|---|---|
| LNPV019-002 | LIPIDV005 | 77 | 0.04 |
| LNPV006-006 | LIPIDV004 | 71 | 0.08 |
| LNPV011-003 | LIPIDV003 | 87 | 0.08 |

The percent encapsulation of luciferase mRNA was measured by the fluorescence-based RNA quantification assay Ribogreen (ThermoFisher Scientific). LNP samples were diluted in 1×TE buffer and mixed with the Ribogreen reagent per manufacturer's recommendations and measured on a i3 SpectraMax spectrophotomer (Molecular Devices) using 644 nm excitation and 673 nm emission wavelengths. To determine the percent encapsulation, LNPs were measured using the Ribogreen assay with intact LNPs and disrupted LNPs, where the particles were incubated with 1×TE buffer containing 0.2% (w/w) Triton-X100 to disrupt particles to allow encapsulated RNA to interact with the Ribogreen reagent. The samples were again measured on the i3 SpectraMax spectrophotometer to determine the total amount of RNA present. Total RNA was subtracted from the amount of RNA detected when the LNPs were intact to determine the fraction encapsulated. Values were multiplied by 100 to determine the percent encapsulation. The Firefly Luciferase mRNA-LNPs that were measured by Ribogreen and the percent RNA encapsulation is reported in Table 25.

TABLE 25
RNA encapsulation after LNP formulation

| LNP ID | Ionizable Lipid | % mRNA encapsulation |
|---|---|---|
| LNPV019-002 | LIPIDV005 | 98 |
| LNPV006-006 | LIPIDV004 | 92 |
| LNPV011-003 | LIPIDV003 | 97 |

### Example 38: In Vitro Activity Testing of mRNA-LNPs in Primary Hepatocytes

In this example, LNPs comprising the luciferase reporter mRNA were used to deliver the RNA cargo into cells in culture. Primary mouse or primary human hepatocytes were thawed and plated in collagen-coated 96-well tissue culture plates at a density of 30,000 or 50,000 cells per well, respectively. The cells were plated in 1× William's Media E with no phenol red and incubated at 37° C. with 5% CO2. After 4 hours, the medium was replaced with maintenance medium (1× William's Media E with no phenol containing Hepatocyte Maintenance Supplement Pack (ThermoFisher Scientific)) and cells were grown overnight at 37° C. with 5% CO2. Firefly Luciferase mRNA-LNPs were thawed at 4° C. and gently mixed. The LNPs were diluted to the appropriate concentration in maintenance media containing 7.5% fetal bovine serum. The LNPs were incubated at 37° C. for 5 minutes prior to being added to the plated primary hepatocytes. To assess delivery of RNA cargo to cells, LNPs were incubated with primary hepatocytes for 24 hours and cells were then harvested and lysed for a Luciferase activity assay. Briefly, medium was aspirated from each well followed by a wash with 1×PBS. The PBS was aspirated from each well and 200 pL passive lysis buffer (PLB) (Promega) was added back to each well and then placed on a plate shaker for 10 minutes. The lysed cells in PLB were frozen and stored at −80° C. until luciferase activity assay was performed.

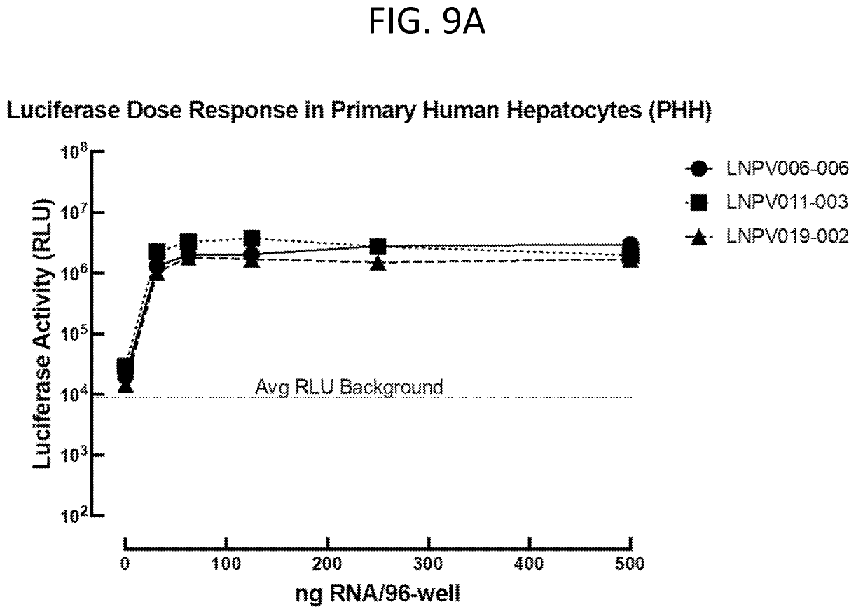

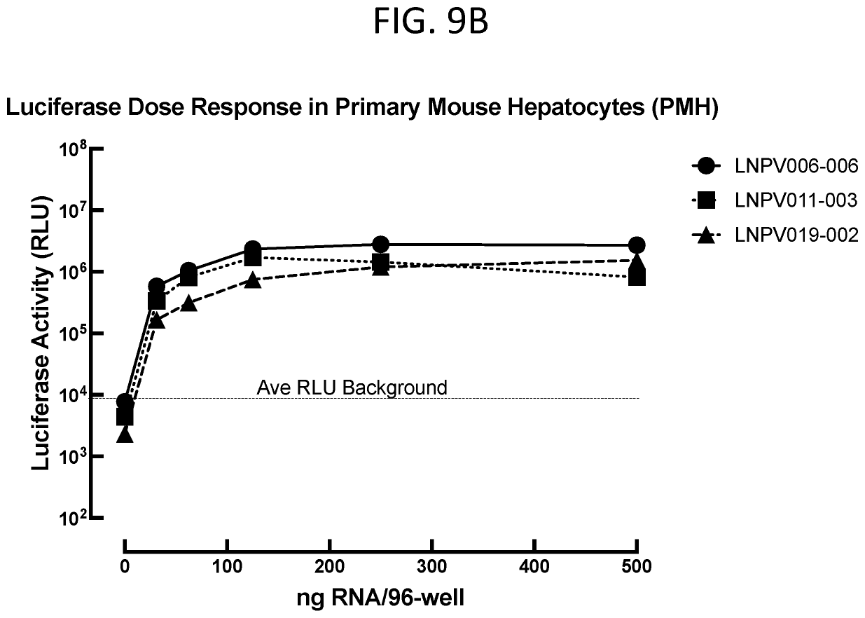

To perform the luciferase activity assay, cellular lysates in passive lysis buffer were thawed, transferred to a round bottom 96-well microtiter plate and spun down at 15,000 g at 4° C. for 3 min to remove cellular debris. The concentration of protein was measured for each sample using the Pierce™ BCA Protein Assay Kit (ThermoFisher Scientific) according to the manufacturer's instructions. Protein concentrations were used to normalize for cell numbers and determine appropriate dilutions of lysates for the luciferase assay. The luciferase activity assay was performed in white-walled 96-well microtiter plates using the luciferase assay reagent (Promega) according to manufacturer's instructions and luminescence was measured using an i3X SpectraMax plate reader (Molecular Devices). The results of the dose-response of Firefly luciferase activity mediated by the Firefly mRNA-LNPs are shown in FIG. 9A and indicate successful LNP-mediated delivery of RNA into primary cells in culture. As shown in FIG. 9A, LNPs were analyzed for delivery of cargo to primary human (FIG. 9A) and mouse (FIG. 9B) hepatocytes. The luciferase assay revealed dose-responsive luciferase activity from cell lysates, indicating successful delivery of RNA to the cells and expression of Firefly luciferase from the mRNA cargo.

### Example 39: LNP-Mediated Delivery of RNA to the Mouse Liver

To measure the effectiveness of LNP-mediated delivery of firefly luciferase containing particles to the liver, LNPs were formulated and characterized and tested in vitro prior to administration to mice. C57BL/6 male mice (Charles River Labs) at approximately 8 weeks of age were dosed with LNPs via intravenous (i.v.) route at 1 mg/kg. Vehicle control animals were dosed i.v. with 300 pL phosphate buffered saline. Mice were injected via intraperitoneal route with dexamethasone at 5 mg/kg 30 minutes prior to injection of LNPs. Tissues were collected at necropsy at or 6, 24, 48 hours after LNP administration with a group size of 5 mice per time point. Liver and other tissue samples were collected, snap-frozen in liquid nitrogen, and stored at −80° C. until analysis.

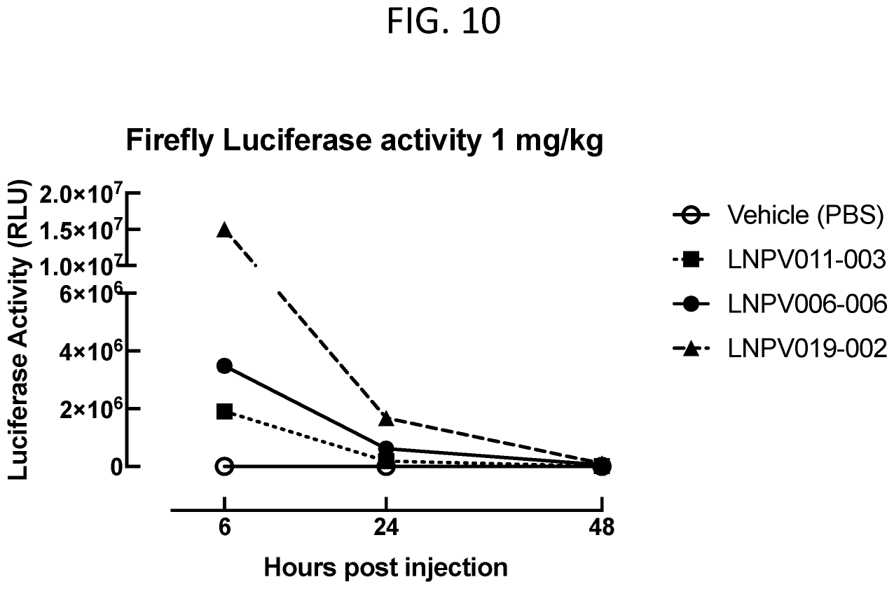

Frozen liver samples were pulverized on dry ice and transferred to homogenization tubes containing lysing matrix D beads (MP Biomedical). Ice-cold 1× luciferase cell culture lysis reagent (CCLR) (Promega) was added to each tube and the samples were homogenized in a Fast Prep-24 5G Homogenizer (MP Biomedical) at 6 m/s for 40 seconds. The samples were transferred to a clean microcentrifuge tube and clarified by centrifugation. Prior to luciferase activity assay, the protein concentration of liver homogenates was determined for each sample using the Pierce™ BCA Protein Assay Kit (ThermoFisher Scientific) according to the manufacturer's instructions. Luciferase activity was measured with 200 pg (total protein) of liver homogenate using the luciferase assay reagent (Promega) according to manufacturer's instructions using an i3X SpectraMax plate reader (Molecular Devices). Liver samples revealed successful delivery of mRNA by all lipid formulations, with reporter activity following the ranking LIPIDV005>LIPIDV004>LIPIDV003 (FIG. 10). As shown in FIG. 10, Firefly luciferase mRNA-containing LNPs were formulated and delivered to mice by iv, and liver samples were harvested and assayed for luciferase activity at 6, 24, and 48 hours post administration. Reporter activity by the various formulations followed the ranking LIPIDV005>LIPIDV004>LIPIDV003. RNA expression was transient and enzyme levels returned near vehicle background by 48 hours. Post-administration. This assay validated the use of these ionizable lipids and their respective formulations for RNA systems for delivery to the liver.

Without wishing to be limited by example, the lipids and formulations described in this example are support the efficacy for the in vivo delivery of other RNA molecules beyond a reporter mRNA. All-RNA Gene Writing systems can be delivered by the formulations described herein. For example, all-RNA systems employing a Gene Writer polypeptide mRNA, Template RNA, and an optional second-nick gRNA are described for editing the genome in vitro by nucleofection, by using modified nucleotides, by lipofection, and for editing cells, e.g., primary T cells. As described in this application, these all-RNA systems have many unique advantages in cellular immunogenicity and toxicity, which is of importance when dealing with more sensitive primary cells, especially immune cells, e.g., T cells, as opposed to immortalized cell culture cell lines. Further, it is contemplated that these all RNA systems could be targeted to alternate tissues and cell types using novel lipid delivery systems as referenced herein, e.g., for delivery to the liver, the lungs, muscle, immune cells, and others, given the function of Gene Writing systems has been validated in multiple cell types in vitro here, and the function of other RNA systems delivered with targeted LNPs is known in the art. The in vivo delivery of Gene Writing systems has potential for great impact in many therapeutic areas, e.g., correcting pathogenic mutations), instilling protective variants, and enhancing cells endogenous to the body, e.g., T cells. Given an appropriate formulation, all-RNA Gene Writing is conceived to enable the manufacture of cell-based therapies in situ in the patient.

### Example 40: Use of an Insulator Configuration Surrounding a DNA Recognition Sequence for Improving the Stability of Transgene Expression after Recombinase-Mediated Integration

In this example, the stability of expression of an integrated transgene is improved through the use of insulator configurations as described in this invention. A DNA recognition sequence comprised on a template nucleic acid will ultimately be split into two terminal recombinase transfer sequences after recombinase-mediated integration. Accordingly, this example describes a configuration in which insulator sequences are located upstream and downstream of the DNA recognition sequence, such that recombination places the insulators inside of the recombinase transfer sequences but at the termini of the inserted DNA. This configuration thus provides an approach by which entire payload of the template nucleic acid, less the DNA recognition sequence, is insulated from incoming chromosomal silencing effects and from outgoing transcriptional activity as described herein (see, e.g., FIGS. 11 and 12).

In order to determine the effect of the insulator configuration on transgene expression, a template nucleic acid either comprising or lacking insulators as described herein is integrated into cells. Briefly, HEK293T and HepG2 cells are nucleofected using varying concentrations of (1) an expression plasmid for producing a recombinase capable of catalyzing recombination using the DNA recognition sequence of (1), e.g., a recombinase comprising the sequence of any of SEQ ID NO: n (e.g., SEQ ID NOs: 1-12,677 (e.g., SEQ ID NOs: 1-11,432)), wherein n is a corresponding number of the DNA recognition sequence included on the template plasmid (e.g., wherein a recombinase of SEQ ID NO: 1 is chosen, a LeftRegion of SEQ ID NO: 13,001 or a right region of SEQ ID NO: 26,001 is chosen); and (2) a template plasmid comprising a reporter cassette, e.g., the constitutive CMV promoter driving EGFP expression, a DNA recognition sequence, e.g., a DNA recognition sequence comprised in a LeftRegion of SEQ ID NO: (n+13,000) or a RightRegion comprising a sequence of SEQ ID NO: (n+26,000), wherein n is chosen from any of 1-12,677 (e.g., any of 1-11,432) (e.g., a sequence of any of SEQ ID NOs: 13,001-25,677 (e.g., SEQ ID NOs: 13,001-24,432) or SEQ ID NOs: 26,001-38,677 (e.g., SEQ ID NOs: 26,001-37,432)), and optionally insulators placed immediately upstream and downstream of the DNA recognition sequence, e.g., the 5′-HS4 chicken β-globin insulator. In some embodiments, the cells used comprise an endogenous target sequence that is also recognized by the recombinase of (1), such that the recombinase is capable of catalyzing the recombination of the DNA recognition sequence of (2) with the endogenous target sequence. In some embodiments, a recombinase without an endogenous target site in the native genome is used for the purpose of experimentation by first integrating an appropriate target site into the genome, e.g., by creating a landing pad comprising the target site. As an example, a landing pad cell line may be generated by transducing HEK293T cells with a lentiviral vector comprising the attB site of the recombinase, e.g., comprising an amino acid sequence of SEQ ID NO: 11,636, comprising the attB sequence 5′-GGCTTGTCGACGACGGCGGTCTCCGTCGTCAGGATCAT-3′. Accordingly, the recombinase expression plasmid encodes the protein sequence of SEQ ID NO: 11,636 and the DNA recognition sequence of the template plasmid comprises a sequence from the LeftRegion or RightRegion comprising the sequence 5′-GGTTTGTCTGGTCAACCACCGCGGTCTCAGTGGTGTACGGTACAAACC-3′.

At three days post-transduction, nucleofection efficiency is determined by measuring EGFP expression by flow cytometry. Subsequently, EGFP expression is similarly assessed on days 7, 10, 14, 21, 28, and 60 post-transduction. At day 21, when plasmid DNA is expected to have been diluted out and GFP fluorescence corresponds to expression from the integrated reporter cassette, a baseline reading of integration frequency is determined by assessing the percent of cells found to be GFP+, while a baseline reading of reporter expression level is determined by assessing median fluorescence, as measured by flow cytometry. The stability of transgene expression is then determined by assessing both the percent of cells remaining GFP+ and the median fluorescence levels at days 28 and 60 post-transduction. Additionally, at days 28 and 60 post-transduction, a portion of cells is first treated with an inhibitor of histone deactylase (HDAC), e.g., trichostatin A (TSA), or an inhibitor of DNA methylation, e.g., 5-azacytidine (5-aza), prior to fluorescence measurements.

In some embodiments, cells treated with template nucleic acid comprising the described insulator configuration will show a decrease in the loss of frequency of expression (e.g., percent GFP+) and/or loss of reporter expression (e.g., median fluorescence) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than cells treated with the template nucleic acid lacking the insulators.

In some embodiments, cells treated with template nucleic acid comprising the described insulator configuration will show a higher frequency of expression (e.g., percent GFP+) and/or a higher level of expression (e.g., median fluorescence) at day 28 and/or day 60 post-transduction, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold higher than cells treated with the template nucleic acid lacking the insulators. In some embodiments, cells treated with template nucleic acid comprising the described insulator configuration will demonstrate a smaller increase in frequency of expression (e.g., percent GFP+) and/or level of expression (e.g., median fluorescence) after further treatment with TSA or 5-aza relative to no treatment with TSA or 5-aza, e.g., at least at least 1.1, 1.2, 1.3, 1.4, 1.5, 1.6, 1.7, 1.8, 1.9, 2.0, 2.5, 3, 4, 5, 6, 7, 8, 9, or at least 10-fold smaller increase than cells treated with the template nucleic acid lacking the insulators. In some embodiments, cells treated with template nucleic acid comprising the described insulator configuration will demonstrate an increase in frequency of expression (e.g., percent GFP+) and/or level of expression (e.g., median fluorescence) after further treatment with TSA or 5-aza of less than 10, 9, 8, 7, 6, 5, 4, 3, 2.5, 2.0, 1.9, 1.8, 1.7, 1.6, 1.5, 1.4, 1.3, 1.2, or less than 1.1-fold increase as compared to no treatment with TSA or 5-aza.

### Example 41: Insulator Configurations for Recombinase-Mediated Transgene Integration Assayed in a 32D Cell-Based Genotoxicity Assay

In this example, the murine myeloblast-like cell line 32D is used as a sensor cell line for genotoxicity. In brief, 32D cells are treated with a genome engineering system, e.g., a recombinase system as described herein, and subsequently grown in vitro in the absence of IL-3 or implanted into mice for in vivo transformation assessment. Genotoxicity of a system is assessed by quantifying the number of colonies capable of IL-3-independent growth (in vitro) or by monitoring implanted mice for the rates of tumor formation and mortality (Li et al Mol Ther 17(4):716-724 (2009), the methods of which are incorporated by reference). Here, the 32D cell line is used to assess genotoxicity in cells treated with template nucleic acid comprising an insulator configuration as described herein, e.g., as described in Example 40.

In order to determine the effect of the insulator configuration on genotoxicity, a template nucleic acid either comprising or lacking insulators as described herein is integrated into cells. Briefly, 32D cells are nucleofected using varying concentrations of (1) a template plasmid and (2) a recombinase expression vector, as described in Example 40 Additionally, cells are nucleofected with (1) only as a negative control or transduced with gammaretrovirus at a target MOI (e.g., MGNP2 at MOI of 1-10) as a positive control. At 24 hr post-transduction, cells are washed and split into 5 independent pools for in vitro transformation assessment, 5 pools for in vivo transformation assessment, and one for transfection or transduction efficiency. First, one subculture is used to determine the transduction efficiency by measuring the fraction of GFP+ cells by flow cytometry at 48 hr post-transduction. The in vitro assessment pools are plated and grown in the absence of IL-3 for quantification of colonies capable of IL-3-independent growth. The remaining 5 subcultures are expanded for ˜2 weeks in the presence of IL-3 before collecting, washing with HBSS, enumerating, and injecting into female C3H/HeJ mice (one independent pool per mouse, 5 mice each). Treated mice are monitored weekly for up to 18 weeks post-transplantation for tumor formation, as previously described.

In some embodiments, the use of a template plasmid comprising an insulator configuration as described herein will result in the formation of fewer IL-3 independent colonies, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold fewer colonies as compared to a template plasmid lacking insulators. In some embodiments, the fraction of mice developing tumors when implanted with cells treated with template plasmid comprising an insulator configuration as described herein will be lower, e.g., at least 20%, 40%, 60%, 80%, or 100% lower than mice implanted with cells treated with a template plasmid lacking insulators. In some embodiments, the median latency of tumors derived from cells treated with a template plasmid comprising an insulator configuration as described herein will be longer, e.g., at least 1.1, 1.2, 1.3, 1.4, 1.5, 1.6, 1.7, 1.8, 1.9, or at least 2-fold longer than those derived from cells treated with a template plasmid lacking insulators. In some embodiments, the 18-week survival rate of mice implanted with cells treated with template plasmid comprising an insulator configuration as described herein will be higher, e.g., at least 1.1, 1.2, 1.3, 1.4, 1.5, 1.6, 1.7, 1.8, 1.9, or at least 2-fold higher than that of mice implanted with cells treated with template plasmid lacking insulators.

### Example 42: Recombinase System with Insulators is Designed to Reduce Interference of Payload on Surrounding Genome-RNA Seg for Transcriptome Analysis

This example describes methods for measuring a reduction in impact of an integrated transgene on the surrounding genome enabled through the use of insulator configurations as described in this invention. Given a genomic integration has the potential to impact expression of endogenous genes in the vicinity of the integration event, it is highly desirable for a therapeutic insertion to have components to minimize this change in local gene expression. Here, the inclusion of an insulator configuration as described herein, e.g., as described in Example 40, in a template nucleic acid is assessed for impact on the change in gene expression relative to a template nucleic acid lacking insulators.

In this example, human primary T cells are nucleofected with systems employing template plasmids comprising insulator configurations as described herein, e.g., Example 40, or template plasmids lacking insulators. Cells are harvested at days 14 and 28 post-nucleofection and RNA is extracted and purified. Briefly, first strand cDNA is generated using the SuperScript® VILO™ cDNA Synthesis Kit (ThermoFisher), as according to manufacturer's instructions, followed by second strand synthesis using DNA Polymerase I. cDNA is purified using Agencourt AMPure XP beads (Beckman Coulter), as according to manufacturer's instructions, and used to prepare DNA libraries by fragmentation, size selection, adaptor ligation, and qPCR quantification. Libraries are sequenced using an Illumina HiSeq 2000 and RNA-seq reads are aligned to reference transcripts to determine a global gene expression profile. Additionally, the expression of specific transcripts located closed to the recombinase-mediated integration site is quantified by qPCR or ddPCR using primers and probes specific to the transcripts.

In some embodiments, integration using a template plasmid comprising an insulator configuration as described herein will result in a change in expression that is lower, e.g., at least 1.1, 1.2, 1.3, 1.4, 1.5, 1.6, 1.7, 1.8, 1.9, 2.0, 2.5, 3, 4, 5, 6, 7, 8, 9, or at least 10-fold lower than the change in expression after integration using a template plasmid lacking insulators for at least one gene local to the site of integration. In some embodiments, integration using a template plasmid comprising an insulator configuration as described herein will result in a less than 10, 9, 8, 7, 6, 5, 4, 3, 2.5, 2.0, 1.9, 1.8, 1.7, 1.6, 1.5, 1.4, 1.3, 1.2, or a less than 1.1-fold change in gene expression for at least one gene local to the site of integration, compared to otherwise similar untreated cells. In some embodiments, integration using a template plasmid comprising an insulator configuration as described herein will result in a less than 10, 9, 8, 7, 6, 5, 4, 3, 2.5, 2.0, 1.9, 1.8, 1.7, 1.6, 1.5, 1.4, 1.3, 1.2, or a less than 1.1-fold change in expression for at least 90%, e.g., at least 90%, 95%, 96%, 97%, 97%, 99%, 99.5% or at least 99.9% of global transcripts, compared to otherwise similar untreated cells.

### Example 43: Identification of attB and attP Attachment Sites for Exemplary Serine Integrases

Putative attB and attP attachment sites were predicted by modeling the biology of phage/host recombinase driven integration. These two DNA sequences are recognized by the serine recombinase enzyme multimeric complex, where attB represents the target integration site on the host and attP represents the donor strand integration cleavage site. To predict these sequence, viral/phage genomes were mapped to their host prokaryote genomes through their respective serine recombinase protein sequences. In any case where a match was found between the serine recombinase in the virus and the host, the recombinase DNA sequence was then used as an anchor for an alignment. Starting with the aligned recombinases, the sequences 5′ and 3′ from the serine recombinase sequence in both virus and host DNA sequences were compared in the pre-integrated viral sequence relative to the post-integrated viral sequence in the prokaryotic host. This process was continued until the sequences ceased to match, thereby mapping the viral genome onto the prokaryotic host. The junctions between the virus and the host were then used to predict a putative host target sequence left and right half attachment sites, attB left (attBL) and attB right (attBR). Also predicted were the viral attachment sites, attP left (attPL) and attP right (attPR). This data is shown in Table 26 below.

TABLE 26

| | | SEQ | | SEQ | | SEQ | | SEQ |
|---|---|---|---|---|---|---|---|---|
| | | ID | | ID | | ID | | ID |
| FL58Accession | attBL | NO | attBR | NO | attPL | NO | attPR | NO |
| MF172979.1:: | TTACATCTTCA | | AAATGATGGG | | TGGAAGAAAT | | TGGAATTCAAG | |
| ASD51140.1 | AGCTCACTTGC | | ACGAAGCTATT | | GACGCGTCTG | | TTAAATATCCT | |
| | AAC | | ATGC | | TAAA | | ACA | |
| YP_006906969.1 | CGCGGTCGCG | | GGGGTCCGGC | | ACCCACGTCA | | GCGAGTCCTTT | |
| | CCGCGTGCGG | | AGGAACAGCC | | GTGGATCTAA | | AGAGACACTG | |
| | TCGTT | | GCCAG | | AGGA | | ACCC | |
| KU517658.1:: | GCTACATAGTC | | CTGCAGTGGCA | | AAATGCTCGG | | TCTTTAATGTA | |
| AMB17413.1 | GCCAGTACCTC | | GCAAAATCAAT | | TACATGAAAA | | GCGCCCAAATG | |
| | CCT | | GTT | | AGCG | | CT | |
| MK448719.1:: | TCAGGTAGTG | | TCCTCATCACTC | | TTAGTCTTTTT | | GTGGTCAGGA | |
| QBX16516.1 | AACCGTTTTTT | | TTTATTTTTTGA | | GGTAGAAGGA | | AATTATCATTT | |
| | GTTG | | A | | ACA | | GAT | |
| MK448825.1:: | AATTAGTAGTG | | AAGACAAGAT | | CTAGTCTTTCT | | AAAATGGTGTT | |
| QBX22171.1 | AACCGTTTTTT | | GTGTCAATTCG | | GATAGAATGA | | CGGATTGATTT | |
| | GTG | | GCAC | | GCA | | ATA | |
| MF172979.1:: | TTACATCTTCA | | AAATGATGGG | | TGGAAGAAAT | | TGGAATTCAAG | |
| ASD51126.1 | AGCTCACTTGC | | ACGAAGCTATT | | GACGCGTCTG | | TTAAATATCCT | |
| | AAC | | ATGC | | TAAA | | ACA | |
| MK448846.1:: | TCTCGTATTGA | | TTATGTGTGCG | | CTAGTCTTTTT | | TTTCGAAACTC | |
| QBX23238.1 | GGTGGCGATTT | | TTGCCTAAAAA | | GATAGAAAAA | | TTAGGTAGAGT | |
| | TTT | | ATG | | GCA | | GTA | |
| MK448720.1:: | AGAATTATATA | | TCTGGCTGTTT | | CTAGTTAATTT | | AAAACGGTCA | |
| QBX16591.1 | ATAATACCCAT | | TTTATGAATGG | | CAAAATATCTC | | GGTTATCGAAA | |
| | GGT | | AAT | | AA | | TATA | |
| MG711462.1:: | TATAAACAACA | | TGCTATATCTG | | TTGTGCATTGT | | AAAACTTACGT | |
| AUV56418.1 | TAGAGCGGTC | | CTATTCTGTTA | | ACACCTTGAT | | TTTTCAGGTGG | |
| | AATA | | CGC | | AAC | | CTT | |
| MG711466.1:: | GCTTTTTGAAA | | CCCCATTCAAG | | ATGATCTATAC | | AGAATCGAACT | |
| AUV56714.1 | AAACGGAGGT | | CGCGCTACCAA | | CGTAGGCGAA | | CACGGCCTCTT | |
| | TTCA | | AAC | | ATG | | GA | |
| MK448934.1:: | CAAATTTATCA | | ATGGCTTTTTT | | TTAATCCCCCT | | AACGAACAAGT | |
| QBX27918.1 | GATTTTTTTGA | | GTGATTAGCAA | | TAACAGTTTCC | | CATATCTATGT | |
| | CAT | | AAG | | AT | | GA | |
| MF595878.1:: | ATTCACAAATG | | GAGCGTTTTTG | | AATGGAATAT | | AAATCCTTTAT | |
| ATB52753.1 | ATCGGGAGGG | | TTTTGCAAAGG | | CATCTGAAAT | | AAATTCAAGGT | |
| | GAAG | | GTA | | CTCG | | TTA | |
| KM983329.1:: | TCTGTACCTAA | | CTATTCCTATT | | TATTTCATTAC | | TAGGGTATTTC | |
| AJA42614.1 | AACATCCATTA | | GGATCTTGAAG | | CTCTTACATGT | | ATTGCCTTCTTT | |
| | AAG | | ATA | | TA | | A | |
| MK448705.1:: | CTATGTCAACG | | AGGATTTTAGT | | TTATAAATTTT | | ATATCAAAATT | |
| QBX15858.1 | ACTTACTCAAA | | GTCATTGATTC | | TTTCACGTTCG | | ATTTTTGATTTT | |
| | CTT | | AAT | | GA | | TA | |
| MK448873.1:: | CGCCCTCTTTA | | AGGATTTTAGT | | TTATCGTTTCC | | ATATCAAAATT | |
| QBX24735.1 | CCAATTATTCA | | GCCATTGATTC | | TAGTCGGGTA | | ATTTTTGATTTT | |
| | GCG | | AAT | | TTT | | TA | |
| MK448940.1:: | TCTTATGAAAA | | AGAAATTTACT | | CTAAACTGGT | | ACATTGCTATC | |
| QBX28214.1 | AGGAGCTTGTC | | ATCCTACATTTC | | TTTTTAGAGAT | | AAGTGGAAGA | |
| | CTT | | AA | | TGT | | TATA | |
| KT337339.1:: | AAAAGCACGCT | | TTCGCTCACAA | | CACCAACATCT | | CTTACCGAATG | |
| ALA47468.1 | GATAATCAGCA | | TAAGAGAGAA | | CAATCAAGGA | | CAACACGAGAT | |
| | AGA | | CTT | | TAG | | CAC | |
| MK448714.1:: | TTTATCCGCAT | | GCGCAAAAAA | | TTATTTAAATG | | ATATCGAGATC | |
| QBX16272.1 | TTTCCGATGCG | | CAAACAAATTT | | TACCCCAAGC | | AAGTGGAGAA | |
| | TTT | | AGAT | | GTC | | TTTG | |
| MK448874.1:: | ATATAACAAAA | | CAAATTTAGTA | | AAATGAAAAA | | GTATCAAAATT | |
| QBX24736.1 | GCGAGCGAGG | | TCATTCATTTCC | | TCCTTTCAAAT | | TTATGGAAAAT | |
| | TAGT | | AC | | ACG | | TTA | |
| MK448927.1:: | AGATACTCTTT | | AGTGTTTAACT | | TTACATTTCCT | | ACATGGAAATT | |
| QBX27562.1 | CCAACCTATTT | | TACACTTATCTC | | CAGCATCAAT | | TTATTTAAAAA | |
| | GTT | | TA | | AAG | | ATA | |
| CP019719.1:: | AGTTCTTGGGG | | GGACATCCTTT | | CTCCTCATGCA | | TCGTATCAATG | |
| QHZ54073.1 | AATCAAATGAA | | GATCGCGTATA | | TATGATAAGT | | GATCCTCATGC | |
| | GAG | | CCG | | AAT | | AT | |
| MK448715.1:: | ATTTTATCAGA | | CCCTGTTTTGC | | TTATGATAGTT | | GCATTGATATT | |
| QBX16366.1 | GTTTCCGCTCT | | TACCGAGTATT | | CGCCCCAAAG | | TTTTTCAATTTT | |
| | GTT | | AAG | | GTC | | TA | |
| MK448826.1:: | GTATATTTAAA | | ATGTATCAAGT | | AATTATTCATA | | GAATTGATATA | |
| QBX22213.1 | GTCTATTTAAA | | GAACACTATTA | | TTTGCCATTTA | | TTTTTGAATTTT | |
| | CAG | | ATA | | AA | | TA | |
| MK448844.1:: | AACACCAGTCT | | CGGTATTGTAT | | TCAAAATGTA | | GCATAGATTTA | |
| QBX23130.1 | TTCGACTGGTG | | CAAGTTAATCA | | TCAAAGTCGT | | ATGCTAGATAT | |
| | CAA | | GAA | | CCGA | | ATA | |
| MK448875.1:: | TAATATCAACG | | AATTACAGTAT | | TTATAAATTTT | | AAATCAACGTT | |
| QBX24786.1 | ACTTACACAAA | | TTAGGAGCACC | | TTTCACGTTCG | | ATTTTTAAATT | |
| | CTT | | TAC | | GA | | GTA | |
| MK448898.1:: | AGACGGTCAA | | AATTACTGTAT | | TTAAGCTTGTA | | AAATCAACGTT | |
| QBX26003.1 | CAGACCGCCTT | | TTAGGAGCACC | | ATTTCATTTCA | | ATTTTTAAATT | |
| | TTAT | | GAC | | AG | | GTA | |
| MK448672.1:: | AAAGATAAAA | | AGCATTATTTA | | AAATGAAAAT | | ATCGATATTAT | |
| QBX14038.1 | CAAAAGAGGT | | GGTACTACAAC | | CAACGTAACA | | TTTTAAGTTTTA | |
| | AAAAC | | TAG | | AGCT | | A | |
| JX507079.1:: | CCCCAGAGAG | | AGGCGGCGGG | | TAAATGGTGG | | AAGGGTTGGC | |
| AFU62848.1 | ACGGGTTTGTC | | AATCGAACCCG | | CGGAACACGG | | CTTGGGTAAAT | |
| | TTTT | | CGTC | | AACC | | GGTG | |
| KX077896.1:: | AGTGGTCCAA | | CGCATATCTCC | | TATGTTAAAG | | GTTTGATAGGT | |
| ANM47644.1 | AAATAGAAAG | | TTAAAAATTTTT | | AAAAACGATA | | TTCTTGATAAG | |
| | TAGAA | | TC | | TTGT | | TCA | |
| MK448674.1:: | CTCAATTGTGA | | AGTAGACGAA | | CTAATCTTTCT | | TAGAGGGGAC | |
| QBX14111.1 | GGTGGCGATTT | | AGGCTGTAACA | | GATAGAATAA | | TGAGGTAGAA | |
| | TTT | | AAAG | | GCA | | TTATA | |
| MK448741.1:: | CTCAATTGTGA | | GTGACTGTGAC | | CTAATCTTTCT | | TAGAGGGGAC | |
| QBX17591.1 | GGTGGCGATTT | | TGAAAGGTTGC | | GATAGAATAT | | AGAGGTAGAC | |
| | TTT | | AGT | | GCA | | TTGTA | |
| MK448752.1:: | CCTTGAAGATG | | AAAAGACAAA | | ATGACAGATA | | AAAATTGAAAA | |
| QBX18231.1 | CGAAAGGACT | | GTCAGTATAGA | | AACAACACGC | | ACTAGTTCATT | |
| | CTAG | | CTTT | | TCTC | | AA | |
| MK448811.1:: | CCTCAATTGCG | | GCACTGATATT | | GCTAATCTTTC | | GAAGAATGGT | |
| QBX21447.1 | AGGTGGCGAT | | TTGGAAAGAAC | | TGATAGAATA | | GACCGGTCATA | |
| | TTTT | | TTA | | AGC | | ATTA | |
| MK448935.1:: | ATCTCAATTGA | | GAGACTGCGA | | TCAGTCTTTTT | | TCGAAGGAAC | |
| QBX27959.1 | GGTGGCGATTT | | CCGAAAGGTTG | | GATAGAAGAA | | AGAAGTTGACT | |
| | TTT | | CGTT | | ACA | | TATA | |
| MK448994.1:: | TCAATTTCTGA | | GGAATTATTCT | | CTAATCTTTCT | | ATGTCCTGGTC | |
| QBX31154.1 | GGTGGCGATTT | | CCTTTCGAATA | | GATAGAATAA | | ACTTAAATTCT | |
| | TTT | | AGA | | GCA | | TCA | |
| MK448999.1:: | CATCTCAATTG | | GATACTGCGAC | | GTCAGTCTTTT | | TGGAAGGAAC | |
| QBX31462.1 | AGGTGGCGAT | | CGAAAGGTTGC | | TGATAGAAGA | | AGAAGTCGAC | |
| | TTTT | | GGT | | GAC | | CTGTA | |
| MN270275.1:: | TTATCTTGGAC | | CGCATATCTCC | | ATATGAACCC | | GTTTGATAGGT | |
| QGJ86433.1 | TATTTTCTTAG | | TTAAAAATTTTT | | TTTCTAATGTG | | TTCTTGATAAG | |
| | CC | | TC | | AGT | | TCA | |
| MN270276.1:: | GTGGTCCCAAA | | TTTTGAGAAGT | | ATGTTAAAGA | | GTGTGAGAAG | |
| QGJ86497.1 | ATAGAAAGTA | | CATCAAAGTAG | | AAAACGATAT | | TGAAGTTGGG | |
| | GAAT | | TAA | | TGTC | | GAATG | |
| MK448713.1:: | CTCAATTACGA | | TAAGTTAAGAA | | CTAGTCTTTTT | | ATTGTGAGTTA | |
| QBX16269.1 | GGTGGCGATTT | | AAAAATGAAAC | | GATAGAAAAA | | GTGGGTACGG | |
| | TTT | | TCA | | GCA | | ATTA | |
| MK448846.1:: | TCTCGTATTGA | | TTATGTGTGCG | | CTAGTCTTTTT | | TTTCGAAACTC | |
| QBX23239.1 | GGTGGCGATTT | | TTGCCTAAAAA | | GATAGAAAAA | | TTAGGTAGAGT | |
| | TTT | | ATG | | GCA | | GTA | |
| MK448719.1:: | TCAGGTAGTG | | TCCTCATCACTC | | TTAGTCTTTTT | | GTGGTCAGGA | |
| QBX16517.1 | AACCGTTTTTT | | TTTATTTTTTGA | | GGTAGAAGGA | | AATTATCATTT | |
| | GTTG | | A | | ACA | | GAT | |
| MK448666.1:: | ATTGGGTAGT | | CTTTAAAAATG | | CTAGTCCTTCT | | AAGAAGATAA | |
| QBX13693.1 | GAACCGTTTTT | | GCTTACGGATT | | GATAGAAGAG | | GAAAATCAGCC | |
| | TGTG | | GAT | | GCA | | TAA | |
| MK448825.1:: | AATTAGTAGTG | | AAGACAAGAT | | CTAGTCTTTCT | | AAAATGGTGTT | |
| QBX22172.1 | AACCGTTTTTT | | GTGTCAATTCG | | GATAGAATGA | | CGGATTGATTT | |
| | GTG | | GCAC | | GCA | | ATA | |

1-2. (canceled)

3. A system for modifying DNA comprising:
a) a template RNA comprising a DNA recognition sequence or a DNA molecule encoding the template RNA;
b) a retroviral structural polypeptide domain;
c) a retroviral reverse transcriptase polypeptide domain capable of reverse transcribing the template RNA, thereby producing a template DNA;
wherein b) and c) are substantially unable to integrate the template DNA into a target DNA;
d) a serine recombinase polypeptide domain, wherein the serine recombinase polypeptide domain binds the DNA recognition sequence and is capable of integrating the template DNA into the target DNA; or a nucleic acid molecule encoding the serine recombinase polypeptide domain; and
e) a retroviral envelope polypeptide domain, or a nucleic acid molecule encoding the retroviral envelope polypeptide domain;
wherein b), c), d), and e) are optionally part of the same polypeptide;
wherein:
(i) the template RNA further comprises a heterologous object sequence encoding a therapeutic effector;
(ii) the DNA recognition sequence of the template DNA is capable of being recombined by the serine recombinase polypeptide domain with a cognate DNA recognition sequence in a naturally occurring human genome and/or in Genome Reference Consortium Human Build 38 (GRCh38), and wherein the target DNA comprises the cognate DNA recognition sequence;
(iii) the serine recombinase polypeptide domain is capable of recombining the DNA recognition sequence of the template DNA with a cognate DNA recognition sequence in a naturally occurring human genome, and wherein the target DNA comprises the cognate DNA recognition sequence; and/or
(iv) the retroviral reverse transcriptase polypeptide domain does not comprise a D64V mutation, or wherein the retroviral reverse transcriptase polypeptide domain comprises a D116 or E152 mutation.

4-7. (canceled)

8. A fusion protein comprising:
one or both of a) a retroviral structural polypeptide domain, and b) a retroviral reverse transcriptase polypeptide domain; and
c) serine recombinase polypeptide domain.

9. A template RNA comprising:
a) a region comprising a DNA recognition sequence that is recognized by a serine recombinase polypeptide domain;
b) a retroviral attachment site;
wherein:
(i) the template RNA further comprises a heterologous object sequence encoding a therapeutic effector; or
(ii) the serine recombinase polypeptide domain comprises an amino acid sequence of any of SEQ ID NOs: 1-12,677, or an amino acid sequence having at least 70%, 75%, 80%, 85%, 90%, 95%, 96%, 97%, 98%, or 99% identity thereto.

10. (canceled)

11. The system of claim 3, wherein the therapeutic effector comprises a polypeptide or functional nucleic acid molecule.

12. The system of claim 11, wherein the functional nucleic acid molecule is an siRNA, lncRNA, asRNA, or miRNA.

13. The system of claim 3, wherein the target DNA is comprised in a human genome.

14. The system of claim 13, wherein the target DNA is present at least once in the human genome.

15. The system of claim 13, wherein the target DNA is present no more than 2 times in the human genome.

16. The system of claim 3, wherein:
(i) the retroviral structural polypeptide domain is a lentiviral structural polypeptide domain;
(ii) the retroviral reverse transcriptase polypeptide domain is a lentiviral reverse transcriptase polypeptide domain; and/or
(iii) the retroviral envelope polypeptide domain is a lentiviral envelope polypeptide domain.

17. The system of claim 3, wherein the system comprises a mutated retroviral integrase.

18. The system of claim 17, wherein the mutated retroviral integrase is a mutated lentiviral integrase.

19. The system of claim 3, wherein the retroviral structural polypeptide domain is a gag domain.

20. The system of claim 3, wherein the retroviral reverse transcriptase polypeptide domain is a pol domain.

21. The system of claim 3, wherein the retroviral envelope polypeptide domain is an env domain.

22. A method of modifying the genome of a cell comprising contacting the cell with the system of claim 3, thereby modifying the genome of the cell.

23. A method of modifying the genome of a cell comprising contacting the cell with the fusion protein of claim 8, thereby modifying the genome of the cell.

24. A method of modifying the genome of a cell comprising contacting the cell with the template RNA of claim 9, thereby modifying the genome of the cell.

25. A lipid nanoparticle (LNP), which comprises the system of claim 3.

26. A system for modifying DNA comprising:
a) a template RNA comprising a DNA recognition sequence, or a DNA molecule encoding the template RNA;
b) a retroviral structural polypeptide domain, or a nucleic acid molecule encoding the retroviral structural polypeptide domain;
c) a retroviral reverse transcriptase polypeptide domain capable of reverse transcribing the template RNA, thereby producing a template DNA, or a nucleic acid molecule encoding the retroviral reverse transcriptase polypeptide domain;
d) a retroviral envelope polypeptide domain, or a nucleic acid molecule encoding the retroviral envelope polypeptide domain;
wherein b) and c) together are integration-deficient or substantially unable to integrate the template DNA into a target DNA;
wherein b), c), and d) are optionally part of the same polypeptide;
wherein:
(i) the DNA recognition sequence is recognized by a serine recombinase polypeptide domain that comprises an amino acid sequence of any of SEQ ID NOs: 1-12,677, or an amino acid sequence having at least 70%, 75%, 80%, 85%, 90%, 95%, 96%, 97%, 98%, or 99% identity thereto; or
(ii) the system further comprises a serine recombinase polypeptide domain comprising an amino acid sequence of any of SEQ ID NOs: 1-12,677, or an amino acid sequence having at least 70%, 75%, 80%, 85%, 90%, 95%, 96%, 97%, 98%, or 99% identity thereto, wherein the serine recombinase polypeptide domain binds the DNA recognition sequence and is capable of integrating the template DNA into the target DNA; or a nucleic acid molecule encoding the serine recombinase polypeptide domain.

27. A method of modifying the genome of a cell comprising contacting the cell with the system of claim 26,
thereby modifying the genome of the cell.
